SEQUENCE LISTING (5 sequences)

SEQ ID NO: 1 - protein, 11 aa, synthetic construct
SDDDMGFGLFD

SEQ ID NO: 2 - DNA, 24 bp, synthetic construct
  source 1..24: mol_type = other DNA
gatgtcggctcttcctatcattgt

SEQ ID NO: 3 - DNA, 23 bp, synthetic construct
  source 1..23: mol_type = other DNA
ccagctcacgttccctattagtc

SEQ ID NO: 4 - DNA, 23 bp, synthetic construct
  source 1..23: mol_type = other DNA
tgccatggtaatcctgctcagta

SEQ ID NO: 5 - DNA, 20 bp, synthetic construct
  source 1..20: mol_type = other DNA
tctgaacctgcggttccaca

(19) United States
(12) Patent Application Publication
(10) Pub. No.: US 2026/0207576 A1
(43) Pub. Date: Jul. 23, 2026

(54) COMPOSITIONS FOR INHIBITING RIBOSOME INACTIVATING PROTEINS (RIPs), METHODS OF IDENTIFYING SAME, AND METHODS OF USE THEREOF

(71) Applicant: Rutgers, The State of University of New Jersey, New Brunswick, NJ (US)

(72) Inventors: Jacques Y. ROBERGE, New Brunswick, NJ (US); Xiao-Ping LI, New Brunswick, NJ (US); Anastasia TSYMBAL, New Brunswick, NJ (US); Nilgun E. Tumer, New Brunswick, NJ (US)

(21) Appl. No.: 19/428,010

(22) Filed: Dec. 19, 2025

Related U.S. Application Data
(60) Provisional application No. 63/737,513, filed on Dec. 20, 2024.

(51) Int. Cl. A61K 31/4535; A61K 31/381; A61K 31/415; A61K 31/422; A61K 31/426; A61K 31/4545; C07D 333/40; C07D 333/80; C07D 409/12; C07D 409/14; C07D 411/14; C07D 417/12; C07D 495/04; G01N 21/64; G01N 33/533

(52) U.S. Cl. CPC A61K 31/4535; A61K 31/381; A61K 31/415; A61K 31/422; A61K 31/426; A61K 31/4545; C07D 333/40; C07D 333/80; C07D 409/12; C07D 409/14; C07D 411/14; C07D 417/12; C07D 495/04; G01N 21/6428; G01N 33/533; G01N 2021/6439

(57) ABSTRACT

The disclosure provides in one aspect a method of treating, ameliorating, and/or preventing toxicity caused by a ribosome inactivating protein (RIP) in a subject. In certain embodiments, the method comprises administering to the subject a therapeutically effective amount of at least one compound of the disclosure, including a compound of Formula (I), Formula (II), or Formula (III):

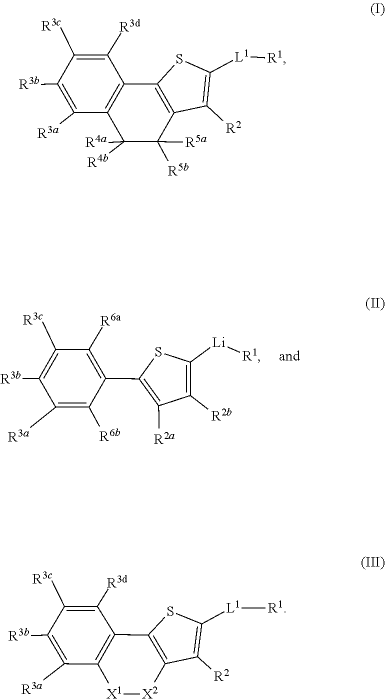

### CROSS-REFERENCE TO RELATED APPLICATIONS

This application claims priority under 35 U.S.C. § 119(e) to U.S. Provisional Patent Application No. 63/737,513, filed Dec. 20, 2024, which is incorporated herein by reference in its entirety.

### STATEMENT REGARDING FEDERALLY SPONSORED RESEARCH OR DEVELOPMENT

This invention was made with government support under grant numbers AI141635, AI072425, and AI178870 awarded by the National Institutes of Health. The government has certain rights in the invention.

### REFERENCE TO AN ELECTRONIC SEQUENCE LISTING

The XML file named “370602-7084US1—Sequence Listing.xml” created on Dec. 18, 2025, comprising 5,599 bytes, is incorporated herein by reference in its entirety.

### BACKGROUND

Ricin produced by castor beans (Ricinus communis) is a worldwide problem as a biothreat agent due to its accessibility, stability, and extreme toxicity, and this compound is classified as a category B agent for bioterrorism. E. coli (STEC), which produce the related Shiga toxins (Stxs) are potentially fatal, foodborne pathogens responsible for the development of hemorrhagic colitis (HC) and hemolytic uremic syndrome (HUS), the leading cause of kidney failure in children. Despite decades of work, there is a dearth of small molecules effective at preventing and/or treating ricin intoxication or STEC infection and as of now only supportive care is available.

There is thus a need in the art for small molecules effective at treating, preventing, and/or ameliorating ricin and/or Shiga toxin intoxication, methods of identifying the same, and methods of use thereof. The present disclosure addresses this unmet need.

### BRIEF SUMMARY OF THE INVENTION

In one aspect, the disclosure provides a compound of Formula (I) or a pharmaceutically acceptable salt thereof, wherein L¹, R¹, R², R^{3a}, R^{3b}, R^{3b}, R^{3d}, R^{4a}, R^{4b}, R^{5a}, and R^{5b} are defined elsewhere herein:

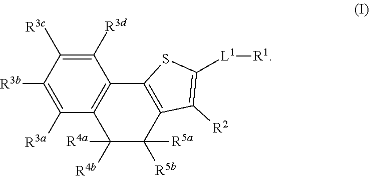

In another aspect, the disclosure provides a compound of Formula (II) or a pharmaceutically acceptable salt thereof, wherein L¹, R¹, R^{2a}, R^{2b}, R^{3a}, R^{3b}, R^{3b}, R^{6a}, and R^{6b} are defined elsewhere herein:

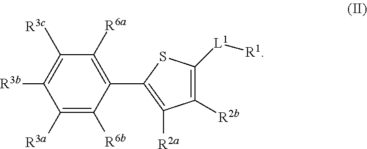

In another aspect, the disclosure provides a compound selected from the group consisting of:
5-(4-fluoro-2,6-dimethylphenyl)thiophene-2-carboxylic acid;
5-(2-bromophenyl)thiophene-2-carboxylic acid;
5-(2-cyclohexylphenyl)thiophene-2-carboxylic acid;
5-(2-hydroxyphenyl)thiophene-2-carboxylic acid;
5-(2,3-dimethylphenyl)thiophene-2-carboxylic acid;
5-(2-methoxy-6-methylphenyl)-3-methylthiophene-2-carboxylic acid;
5-(4-amino-2,6-dimethylphenyl)thiophene-2-carboxylic acid;
5-(2-ethyl-6-(5-methylthiophene-2-carbonyl)phenyl)thiophene-2-carboxylic acid;
5-(2-ethyl-6-((5-methylthiophen-2-yl)methyl)phenyl)thiophene-2-carboxylic acid;
5,5-dimethyl-4-oxo-4,5-dihydronaphtho[1,2-b]thiophene-2-carboxylic acid;
5-(2-ethylphenyl)-4-methylthiophene-2-carboxylic acid;
5-(2-ethylphenyl)-1-methyl-1H-pyrazole-3-carboxylic acid; 5-(2-ethylphenyl)-1H-pyrazole-3-carboxylic acid;
methyl 3-(5,5-dimethyl-4,5-dihydronaphtho[1,2-b]thiophen-2-yl)-2,2-dihydroxy-3-oxopropanoate;
methyl 5-(5-bromo-2-methylphenyl)thiophene-2-carboxylate;
1-(4-methyl-3-nitropyridin-2-yl)-4-((4-methylthiazol-2-yl)methyl)piperidin-4-ol;
5-(2-bromo-6-methylphenyl)thiophene-2-carboxylic acid; and methyl 5-(2-bromo-6-methylphenyl)thiophene-2-carboxylate.

In another aspect, the disclosure provides a pharmaceutical composition comprising the compound of the disclosure and a pharmaceutically acceptable carrier.

In another aspect, the disclosure provides a method of treating, preventing, and/or ameliorating toxicity caused by a ribosome inactivating protein (RIP) in a subject, the method comprising administering to the subject a therapeutically effective amount of at least one compound of the disclosure.

In another aspect, the disclosure provides a method for measuring binding affinity of a small molecule for an active A chain (RTA) of a ribosome inactivating protein (RIP), the method comprising:
(a) contacting a small molecule with a mixture comprising a fluorescently labeled P11 polypeptide (SEQ ID NO:1) and RTA to provide a displacing mixture;
(b) measuring fluorescence of the displacing mixture to provide a displaced fluorescence mixture;
(c) measuring fluorescence of a control sample comprising the fluorescently labeled P11 polypeptide (SEQ ID NO:1) and RTA, and lacking the small molecule, to provide a control measurement; and
(d) comparing the displaced fluorescence measurement and the control fluorescence measurement.

### BRIEF DESCRIPTION OF THE FIGURES

The drawings illustrate generally, by way of example, but not by way of limitation, various embodiments of the present application.

FIG. 1: Model showing the interaction of RTA with the P-stalk to access the SRL. The S. cerevisiae 60S subunit and 26S rRNA (PDB ID: 4V88) are shown in green and gray. The fitted structure shows the uL10 fragment in complex with the NMR structure of the P1-P2 heterodimers from H. sapiens (PDB ID: 4BEH) in shades of light grays and blues, interacting with RTA (PDB ID: 5GU4) in white. The hydrophobic pocket at the P-stalk binding site of RTA is colored cyan and the active site of RTA and the SRL substrate, where the adenine is removed are both colored red.

FIG. 2: Synthesis of new analogs of PD00589. i. DMF, POCl₃, 70° C.; ii. Pyridine, methyl thioglycolate, Et₃N, 60° C.; iii. 1 M NaOH, MeOH, THF; iv (c) MeB(OH)₂, Pd(OAc)₂, PCy₃, K₃PO₄, toluene:water; iv (d) Me₂NH, Pd(OAc)₂, BINAP, Cs₂CO₃, dioxane; iv (e) BrettPhosPdG3, Cs₂CO₃, dioxane:MeOH.

FIG. 3: Synthesis of RU-NT-206 analogs. i. NH₄Cl, HATU, (i-Pr)₂NEt, DMF; ii. Burgess reagent, DCM.

FIG. 4: Different thiophene ring orientations. The relatively different thiophene ring orientations in RU-NT-93 (red sticks) relative to CC10501 (split pea green sticks) when bound to the P stalk pocket of RTA (green sticks). All nitrogen atoms are colored blue, all oxygen atoms are colored red, and all sulfur atoms are colored yellow.

FIG. 5A: Direct binding of BODIPY-TMR labeled P11 peptide to RTA. The concentration of P11 peptide is fixed at 1 μM. The anisotropy values were obtained using excitation and emission bandpass filters 530/25 nm and 590/35 nm, respectively. Fraction bound of labeled P11 is calculated using equation 4 and plotted as a function of RTA concentration (μM). The binding constant (K_{D}) is obtained by using the quadratic equation 6 for non-linear curve-fitting analysis. FIGS. 5B-5C: Competitive displacement of bound labeled P11 peptide from RTA is carried out in the presence of PT peptide (FIG. 5B) and a small molecule BTB13068 (FIG. 5C), which do not bind at the P-stalk site of RTA as negative controls. Two different colors were used for two different biological replicates.

FIGS. 6A-6F: Fluorescence polarization was used to measure the competitive displacement of BODIPY TMR-X-labeled P11 from RTA by the unlabeled P11 peptide (FIG. 6A) and small molecule inhibitors (FIGS. 6B-6F). Reactions containing labeled P11 (1 μM), and RTA (3 μM) were incubated with varying concentrations of inhibitors in 40 μL of 1X FP buffer (25 mM Tris-HCl pH 8.0 and 100 mM NaCl) for 30 min in the dark at room temperature followed by centrifugation at 400 g for two minutes and scanned using BioTek Synergy 4 microplate reader. Anisotropy values were obtained using 530/25 nm excitation bandpass and 590/35 emission filters. Normalized values for the percentage of binding (%) were plotted against the inhibitor concentration and the IC₅₀ values obtained by FP were used to calculate the K₁ values as described herein. The different colored data points represent 4 different measurements.

FIGS. 7A-7D: In vitro depurination inhibition. The percentage of inhibition of the RTA-mediated depurination by the small molecule inhibitors was measured by qRT-PCR using rat liver ribosomes. The different colored data points represent different biological replicates. The data for the percentage of inhibition at different compound concentrations were fitted with Michaelis-Menten kinetics using OriginPro 2023 to calculate the 50% inhibitory activity (IC₅₀).

FIGS. 8A-8D: Inhibition of RTA-mediated depurination of yeast ribosomes. QRT-PCR measured the percentage of inhibition of depurination. The different colored data points represent different biological replicates. The data for the percentage of inhibition at different compound concentrations were fitted with Michaelis-Menten kinetics using OriginPro 2023 to calculate the IC₅₀.

FIGS. 9A-9F: Structures of RTA-inhibitor complexes. The structure of RTA (green) is depicted as a ribbon diagram in complex with inhibitors (FIG. 9A) RU-NT-59 (magenta), (FIG. 9B) PD00589 (cyan), and (FIG. 9C) RU-NT-206 (green). RTA active site residue Tyr80 is drawn as sticks and colored red. 2Fo-Fc (blue mesh) and Fo-Fc (red mesh) electron density maps of (FIG. 9D) RU-NT-59, (FIG. 9E) PD00589, and (FIG. 9F) RU-NT-206. The original 2Fo-Fc and Fo-Fc electron density maps were contoured at 1.0 σ and 3.0 σ levels, respectively. The maps were calculated before each inhibitor was built into the density maps. Each inhibitor is drawn as sticks with all carbon atoms in RU-NT-59 colored magenta and all carbon atoms in PD00589 colored cyan. All nitrogen oxygen atoms are colored red and sulfur atoms yellow.

FIG. 10: The relative position of RU-NT-59 (magenta sticks), PD00589 (cyan sticks), and RU-NT-206 (green sticks) bound to the P stalk pocket of RTA (green sticks). All nitrogen atoms are colored blue, all oxygen atoms are colored red, and all sulfur atoms are colored yellow.

FIGS. 11A-11D: Key inhibitor interactions with RTA. Zoom-in of the noncovalent interactions of RTA (green) in complex with (FIG. 11A, FIG. 11C) RU-NT-59 (magenta), (FIG. 11B) PD00589 (cyan), and (FIG. 11D) RU-NT-206 (green) drawn as sticks. All nitrogen atoms are colored blue, all oxygen atoms are colored red, and all sulfur atoms are colored yellow. The salt-bridge and H-bond are represented as red dashes with the similar nonpolar contacts between RTA and each inhibitor is represented as yellow dashes. The additional contacts between the fluoride atom in RU-NT-59 and RTA are colored cyan dashes in (FIG. 11C).

FIG. 12: Similar structures of RTA bound to different inhibitors. Shown are the super positioned Cα-traces of RTA when bound to RU-NT-59, PD00589, RU-NT-206, P11, and in the apo form of RTA colored from dark gray to light gray.

FIGS. 13A-13B: Inhibition of ribosome depurination by ricin holotoxin in Vero cells. FIG. 13A: Vero cells were plated at 1.5×10⁵/mL and grown for 24 h. Cells were treated with each compound and 200 pM ricin as described herein. The percentage of depurination was measured by qRT-PCR compared to DMSO-treated cells at 2 h. Data analysis using ANOVA and Dunnett's test was used to determine the significance of the effect of the different compounds on the percentage of inhibition of depurination of cells treated with ricin compared to the percentage of inhibition of depurination of cells treated with ricin but with no compound (control). *** p<0.001 FIG. 13B: The half maximal effective concentration (EC₅₀) value for inhibition of depurination by ricin holotoxin in Vero cells was determined by qRT-PCR. The data for the percent inhibition at different fragment concentrations were fitted with Michaelis-Menten kinetics using OriginPro 2023. Data is from 10 different biological replicates shown in different colors.

FIGS. 14A-14B: Inhibition of ribosome depurination by ricin holotoxin in A549 cells. FIG. 14A: A549 cells were plated at 1.5×10⁵/mL and grown for 24 hours. Cells were treated with each compound, followed by 200 pM ricin as described herein. The percentage of depurination was measured by qRT-PCR compared to DMSO treated cells at 2 h. Data analysis using ANOVA and the Dunnett's test was used to determine the significance of the effect of the different compounds on the mean % inhibition of depurination of cells treated with ricin compared to the % inhibition of depurination of cells treated with ricin but with no compound (the control). * * * p<0.001 FIG. 14B: The half maximal effective concentration (EC₅₀) for inhibition of depurination by ricin holotoxin was determined by qRT-PCR. The data for the percentage of inhibition at different compound concentrations were fitted with Michaelis-Menten kinetics using OriginPro 2023.

FIGS. 15A-15B: Inhibition of ribosome depurination activity of Stx2A1 and Stx2a holotoxin by RU-NT-206 in Vero cells. FIG. 15A: In vitro depurination inhibition by RU-NT-206 against Stx2A1 using rat liver ribosomes. The percentage of inhibition was measured by qRT-PCR. The different colored data points represent different biological replicates. The IC₅₀ value against Stx2A1 was determined by qRT-PCR using yeast and rat liver ribosomes. The data for the percentage of inhibition at different fragment concentrations were fitted with Michaelis-Menten kinetics using OriginPro 2023 to determine the IC₅₀. FIG. 15B: Vero cells were plated at 1.5×10⁵/mL and grown for 24 h. Cells were treated with RU-NT-206 and 200 pM ricin or 2 nM Stx2a as described herein. The percentage of depurination was measured by qRT-PCR compared to DMSO-treated cells at 2 h. ANOVA and Dunnett's test were used to determine if the compound significantly reduced the percentage of inhibition of depurination in cells treated with toxin compared with cells treated with toxin and no compound (the control). NS p >0.05, * * * p 0.001.

FIGS. 16A-16C: P11 peptide contacts with RTA and similar binding mode of CC10501 and P11 with RTA. FIG. 16A: Close-up of the noncovalent interactions between RTA (green) in complex with the P11 peptide (grey). RTA and P11 are all drawn as sticks. Salt-bridge is represented as red dashes with π-stacking and nonpolar contacts are represented as yellow dashes. The P11 primary sequence is depicted in bold text. The non-covalent interactions of RTA (green) in complex with FIGS. 16B-16C: CC10501 (split-pea green, FIG. 16B) and the RTA-CC10501 (FIG. 16C) complex superposed with the P11 peptide (gray) showing the similar binding within the P stalk pocket. All molecules are drawn as sticks. All nitrogen atoms are colored blue, all oxygen atoms are colored red, and all sulfur atoms are colored yellow. The salt-bridge is represented as red dashes with the nonpolar contacts between RTA and each inhibitor represented as yellow dashes.

FIGS. 17A-17C: Key interactions of RU-NT-93 with RTA in the P stalk pocket of RTA influencing inhibition. FIG. 17A: Position of the 2,6-dimethylphenyl ring of RU-NT-93 (red) relative to the benzene ring in CC10501 (split pea green) within the P-stalk pocket of RTA drawn as a gray molecular surface. FIG. 17B: RTA (green) bound to RU-NT-93 (red) with the superposed P11 (gray) depicting the similar space occupied by the 2,6-dimethylphenyl ring with the Phe10 and Leu9 of the P11 peptide. FIG. 17C: RTA (green) bound to RU-NT-93 (red) superposed with RU-NT-206 (green) and P11 (gray) depicting the similar space occupied by the 2,6-dimethylphenyl ring in RU-NT-93 and benzene ring in RU-NT-206 and the P11 peptide Phe10 and Leu9 residues. All molecules are drawn as sticks. All nitrogen atoms are colored blue, all oxygen atoms are colored red, and all sulfur atoms are colored yellow.

FIGS. 18A-18B: Interaction of RU-NT-59, PD00589, and RU-NT-206 with the P-stalk pocket of RTA. FIG. 18A: The comparable binding mode of RU-NT-59 (magenta sticks), PD00589 (cyan sticks), and RU-NT-206 (green sticks) to the P-stalk pocket with the P11 peptide (gray sticks) superposed depicting each inhibitor directly blocking the interaction of Leu9, Phe10, and Asp11 with RTA. FIG. 18B: The hydrophobic pocket within the P11 binding site on RTA is depicted as a gray surface. Key RTA residues lining this hydrophobic pocket were drawn as green sticks. RU-NT-206 is drawn as green sticks. The C1 and C3 methyl groups on RU-NT-206 are labeled C1 and C3, respectively. The yellow dashes depict the hydrophobic contact between the C1 and C3 methyl groups on RU-NT-206 and Leu207, Leu232, and I1e251 from RTA. All nitrogen atoms are colored blue, all oxygen atoms are colored red, and all sulfur atoms are colored yellow.

FIG. 19: High resolution mass spectrometry analysis of the unlabeled P11 peptide probe. The theoretical mass-charge ratio (m/z) of the P11 peptide (SDDDMGFGLFD) is 1218.46195 and the observed m/z is 1218.4591 with a mass error of −2.34 ppm.

FIG. 20: High resolution mass spectrometry analysis of the BODIPY TMR-X labeled P11 peptide probe. The theoretical mass-charge ratio (m/z) of MNa+ BODIPY TMR-X labeled P11 peptide (SDDDMGFGLFD) is 1733.67872 and the observed m/z is 1733.6787 with a mass error of −0.33 ppm.

FIGS. 21A-21D: Inhibition of rat ribosome depurination by RTA with administration of CC10501 (FIG. 21A), RU-NT-93 (FIG. 21B), RU-NT-192 (FIG. 21C), and RU-NT-206 (FIG. 21D).

FIGS. 22A-22C: Inhibition of rat ribosome depurination by Stx2A1 with administration of CC10501 (FIG. 22A), RU-NT-192 (FIG. 22B), and RU-NT-206 (FIG. 22C).

FIGS. 23A-23B: inhibition of ricin and Stx2a-mediated ribosome depurination in Vero cells with administration of RU-NT-192 (FIG. 23A) and RU-NT-206 (FIG. 23B).

FIGS. 24A-24D: Cell protection (viability) against ricin holotoxin by RU-NT-165 (FIG. 24A), RU-NT-192 (FIG. 24B), RU-NT-202 (FIG. 24C), and RU-NT-124 (FIG. 24D) and Vero cell viability was determined using the CellTiter-Glo® Luminescent Cell Viability Assay (Promega) which measures the ATP content released from lysed cells. The data represent three biological replicates.

FIGS. 25A-25C: EC₅₀ of compounds RU-NT-206 (FIG. 25A), RU-NT-255 (FIG. 25B), and RU-NT-256 (FIG. 25C) with incubation of ricin in Vero cells analyzed by the Hill equation. The Hill coefficient (n) around 1 indicates 1:1 interaction with RTA.

FIGS. 26A-26B: Inhibition of ricin (FIG. 26A) and Stx2a (FIG. 26B) mediated ribosome depuration by RU-NT-206, RU-NT-255, and RU-NT-256 in Vero cells.

FIGS. 27A-27B: exemplary ricin and Shiga toxin (Stx2) inhibition data for certain compounds of the disclosure.

FIGS. 28A-28F: Inhibition of RTA-mediated depurination of rat liver ribosomes by CC10501 (FIG. 28A), RU-NT-135 (FIG. 28B), RU-NT-202 (FIG. 28C), RU-NT-165 (FIG. 28D), RU-NT-102 (FIG. 28E), and RU-NT-124 (FIG. 28F). Compounds at different concentrations were mixed with RTA first and the ribosomes were added to start the reaction. The reaction was incubated at room temperature for 5 min. RNA extraction buffer was added to stop the reaction. RNA was extracted and the level of inhibition was determined by qRT-PCR. Measurements were repeated 2-4 times as indicated by the different symbols. The data for the percentage of inhibition at different compound concentrations were fitted with the Hill equation using OriginPro 2023 to calculate the IC₅₀.

FIGS. 29A-29F: Inhibition of ribosome depurination by ricin holotoxin in Vero cells by RU-NT-135 (FIG. 29A), RU-NT-202 (FIG. 29B), RU-NT-102 (FIG. 29C), RU-NT-165 (FIG. 29D), RU-NT-124 (FIG. 29E), and RU-NT-192 (FIG. 29F). Vero cells were plated at 1.5×10⁵/mL and grown for 24 hours. Cells were treated with each compound and 200 pM ricin as described in the Methods. The percentage of depurination was measured by qRT-PCR compared to DMSO-treated cells at 2 h. The half maximal effective concentration (EC₅₀) value for inhibition of depurination by ricin holotoxin in Vero cells was determined by fitting the data with the Michaelis Menten equation for RU-NT-135 and the Hill equation for the other compounds using OriginPro 2023. Data are from 3-19 biological replicates shown in different colors.

FIGS. 30A-30E: Analysis of RU-NT-192 binding site in RTA using chemical shift perturbation (CSP) data obtained from NMR experiments acquired at 700 MHz spectrometer and 25° C. temperature. FIGS. 30A-30B: Methyl ¹H-¹³C HMQC (FIG. 30A) and Amide ¹H-¹⁵N HSQC (FIG. 30B) in the free state of RTA (black contours) overlaid with the inhibitor-bound complex (red contours). FIGS. 30C-30D: Backbone CSPs mapped on the X-ray structure of RTA (PDB code 1RTC) in two different orientations. Residues with amide CSPs >0.1 ppm are highlighted in orange and those that disappear in the complex due to exchange broadening are painted red. Annotated methyl groups with CSP >0 from Panel A, substrate binding and catalytic site residues (green) are shown in line representation. FIG. 30E: Residue-specific profile of the weighted average of the amide proton (¹H) and nitrogen (¹⁵N) chemical shift differences between the free state and inhibitor complex calculated using the relationship √0.5*[(Dd_{H}^{N})²+(0.2*Dd_{N})²]. The exchange-broadened sites in the complex are indicated by red lines.

FIGS. 31A-31E: Analysis of RU-NT-206 binding site in RTA using chemical shift perturbation (CSP) data obtained from NMR experiments acquired at 700 MHz spectrometer and 25° C. temperature. FIGS. 31A-31B: Methyl ¹H-¹³C HMQC (FIG. 31A) and Amide ¹H-¹⁵N HSQC (FIG. 31B) in the free state of RTA (black contours) overlaid with the inhibitor-bound complex (red contours). FIGS. 31C-31D: Backbone CSPs mapped on the X-ray structure of RTA (PDB code 1RTC) in two different orientations. Residues with amide CSPs >0.1 ppm are highlighted in orange and those that disappear in the complex due to exchange broadening are painted red. Annotated methyl groups with CSP >0 (Panel A) and the catalytic site residues (green) are shown in line representation. FIG. 30E: Residue-specific profile of the weighted average of the amide proton (¹H) and nitrogen (¹⁵N) chemical shift differences between the free state and inhibitor complex calculated using the relationship √0.5*[(Dd_{H}^{N})₂+(0.2*Dd_{N})²]. The exchange-broadened sites are shown by red lines.

FIG. 32: The Kᵢ values of RU-NT-06, RU-NT-192, RU-NT-165, RU-NT-135, and RU-NT-202 from the FA assay and their EC₅₀ values from the Vero cell-based assay are directedly proportional.

FIGS. 33A-33C: Inhibition of Stx2A1-mediated depurination of rat liver ribosomes by small molecule inhibitors CC0501 (FIG. 33A), RU-T-192 (FIG. 33B), and RU-NT-206 (FIG. 33C). Measurements were repeated 2-6 times as indicated by different symbols. The different colored data points represent different biological replicates. The data for the percentage of inhibition at different compound concentrations were fitted with the Hill equation using OriginPro 2023 to calculate the 50% inhibitory activity (IC₅₀).

FIG. 34: Inhibition of ribosome depurination activity of ricin and Shiga toxin 2a (Stx2a) by RU-NT-192 in Vero cells. Vero cells were incubated with varying concentrations of RU-NT-192 for 2 hours (for ricin) or 3 hours (for Stx2a) at 37° C., in the presence of 200 pM ricin or 2 nM Stx2a. The level of depurination was measured using a quantitative PCR-based assay. For each experiment, three biological replicates were used. Data for ricin are presented as the mean standard deviation of the three replicates from two independent experiments. Data for Stx2a are presented as the mean±standard deviation of three replicates from a single experiment.

FIG. 35: Summarized medicinal chemistry effort towards improvement of RU-NT-192.

FIG. 36: Table showing affinity and potency of exemplary RU-NT-192 analogs against ricin. The Kᵢ values were measured by fluorescence anisotropy (FA). The IC₅₀ values by FA represent the displacement of fluorescent P11 probe from RTA. The IC₅₀ values by qRT-PCR represent in vitro depurination of rat liver ribosomes. Data were analyzed using the Hill equation. “nH” is the Hill coefficient. The EC₅₀ values were determined by qRT-PCR in Vero cells using ricin holotoxin and analyzed using the Hill equation. Results represent at least 2-3 biological replicates of each assay. “tet” is 1-H-tetrazole. “U.D.” unable to determine. “NT” not tested.

FIGS. 37A-37B: Inhibitor binding modes within the P-stalk pocket of RTA. FIGS. 37A-37B: RU-NT-323 (FIG. 37A) and RU-NT-422 (FIG. 37B) bind within the P-stalk pocket. Nitrogen atoms are blue, oxygen atoms are red, and sulfur atoms are yellow. The H-bonds are shown as red dashes, salt-bridges as yellow dashes, and all nonpolar contacts are shown as gray dashes. The π-π stacking interactions are drawn as blue dashes.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

Ribosome binding sites of ricin or Shiga toxin have not been targeted by small molecules. Previous studies identified CC10501, which binds at the P-stalk pocket of RTA and inhibits activity. In one aspect, the disclosure provides a new fluorescence polarization assay and demonstrates that compounds which bind at the P-stalk pocket of RTA with higher affinity cause more potent inhibition of the catalytic activity of ricin. Structure-activity relationships established that the presence of the carboxylic acid, or a bioisostere thereof, is essential for improved affinity for the P-stalk pocket of RTA. A lead compound, RU-NT-206, bound at the P-stalk pocket in a unique binding mode with similar affinity against RTA as a five-fold larger P-protein peptide and protected cells against ricin and Shiga toxin 2 for the first time. Additionally, exemplary analogues thereof (e.g., RU-NT-253, RU-NT-254, RU-NT-255, and RU-NT-256) have been designed, synthesized, and evaluated. Importantly, certain compounds of the disclosure (e.g., RU-NT-192) permit toxicity studies previously unavailable. These studies validate the ribosome-binding site of ricin as a critical target for allosteric inhibition of the catalytic site and demonstrate that certain exemplary compounds can effectively treat, prevent, and/or ameliorate associated toxicity.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C═C(CH₃), —C═C(CH₂CH₃), —CH₂C═CH, —CH₂C═C(CH₃), and —CH₂C═C(CH₂CH₃) among others.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, biphenylenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3—, 4-, 5-, or 6-positions of the phenyl ring, or a naphthyl group substituted at any one or more of 2- to 8-positions thereof.

The term “bioisostere” refers to a surrogate structure that exhibits broadly similar biological properties as an atom, or group of atoms of a biologically active compound. Thus, the term “carboxylic acid bioisostere” refers to a surrogate structure that exhibits broadly similar biological properties as a carboxylic acid moiety of a biologically active compound. Non-limiting examples of carboxylic acid bioisosteres are the carboxylic acid bioisosteres described in C. Ballatore et al., ChemMedChem 2013, 8, 385-395 (DOI: 10.1002/cmdc.201200585) and in P. Lassalas et al., J. Med. Chem. 2016, 59, 3183-3203 (DOI: 10.1021/acs.jmedchem.5b01963). Non-limiting, exemplary carboxylic acid bioisosteres include hydroxamic acids, hydroxamic esters, phosphonic acids, phosphinic acids, sulfonic acids, sulfonamides, acyl sulfonamides, sulfonylureas, acylureas, tetrazoles, thiazolidine diones, oxazolidine diones, and oxadiazol-5(4H)-ones, inter alia.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkylene” or “cycloalkylenyl” as used herein refers to a bivalent saturated cycloalkyl radical (e.g.,

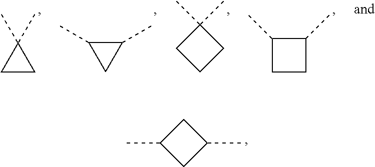

inter alia). In certain embodiments, the term may be regarded as a product of removal of two hydrogen atoms from the corresponding cycloalkane (e.g., cyclobutyl) by removal of two hydrogen atoms from the same (e.g.,

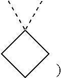

different (e.g.,

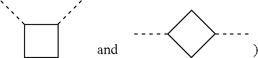

carbon atoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “ameliorated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical (e.g.,

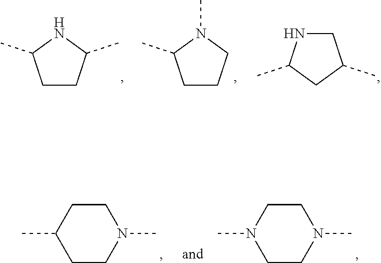

inter alia). In certain embodiments, the term may be regarded as a product of removal of two hydrogen atoms from the corresponding heterocycloalkane (e.g., piperidine) by removal of two hydrogen atoms from the same (e.g.,

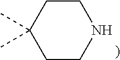

different (e.g.,

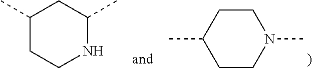

carbon atom(s) and/or heteroatom(s).

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3—, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “specifically binds”, or “specifically binds”, or the like, means that an antibody or antigen-binding fragment forms a complex with an antigen that is relatively stable under physiological conditions. The specific bond can be characterized by an equilibrium dissociation constant of at least about 5×10⁻⁸ M or less (for example, a smaller K_{D} denotes a firmer bond). Methods for determining whether two molecules specifically bind to each other are well known in the art and include, for example, equilibrium dialysis, surface plasmon resonance, and the like. As described in this document, antibodies have been identified by surface plasmon resonance, for example, BIACORE™, which specifically binds to CD8

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The terms “treat,” “treating” and “treatment,” as used herein, means reducing the frequency or severity with which symptoms of a disease or condition are experienced by a subject by virtue of administering an agent or compound to the subject.

### Compounds

In one aspect, the disclosure provides a compound of Formula (I) or a pharmaceutically acceptable salt thereof:

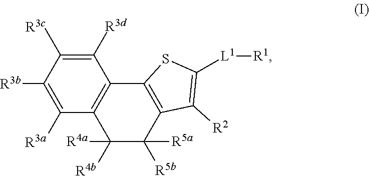

wherein:
   R¹ is a carboxylic acid (—COOH), —C(═O)C(OH)₂C(═O)OR^{A}, a carboxylic acid bioisostere, or a carboxylic acid precursor;
   R² is H;
   R^{3a}, R^{3b}, R^{3b}, and R^{3d} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   L¹ is selected from the group consisting of a bond and optionally substituted C₁-C₂ alkylenyl;
   R^{4a} and R^{4b} are each independently selected from the group consisting of H and optionally substituted C₁-C₆ alkyl,
      wherein no more than one of R^{4a} and R^{4b} is H;
   R^{5a} and R^{5b} are each independently selected from the group consisting of H and optionally substituted C₁-C₆ alkyl,
      or R^{5a} and R^{5b} can combine with the carbon atom to which they are bound to form a carbonyl moiety (C═O);
   R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl.

In certain embodiments, the compound is not 5,5-dimethyl-4,5-dihydronaphtho[1,2-b]thiophene-2-carboxylic acid.

In certain embodiments, R¹ is selected from the group consisting of

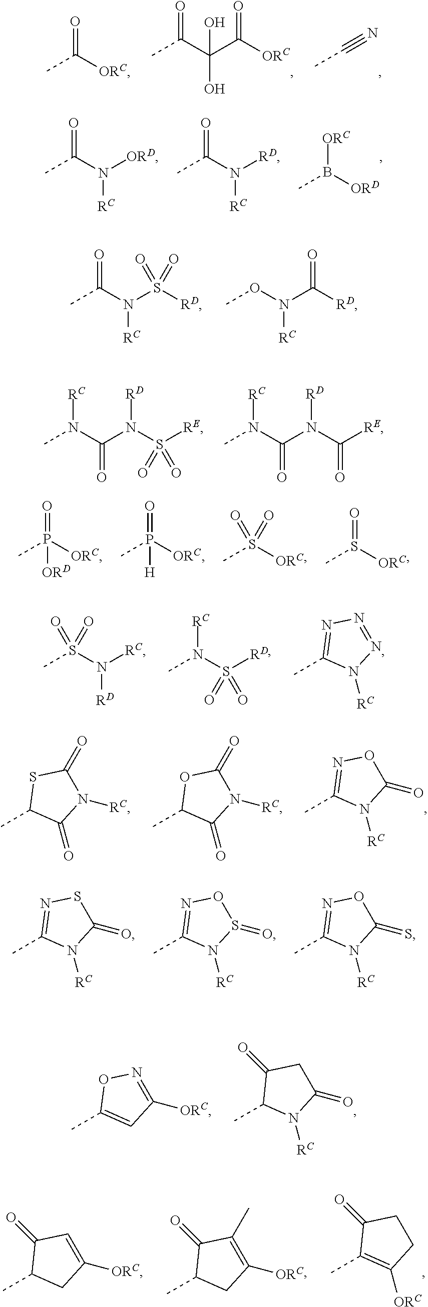

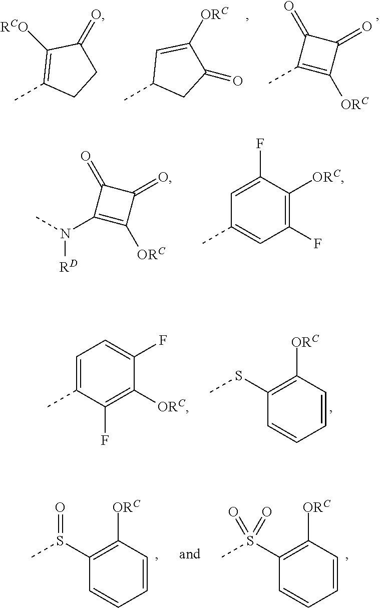

wherein R^{C}, R^{D}, and R^{E}, if present, are each independently selected from the group consisting of H, optionally substituted C₁-C₆ alkyl, and C(═O)(optionally substituted C₁-C₆ alkyl).

In certain embodiments, R¹ is

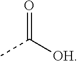

In certain embodiments, R¹ is

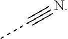

In certain embodiments, R¹ is

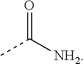

In certain embodiments, R¹ is

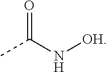

In certain embodiments, R¹ is

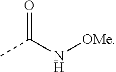

In certain embodiments, R¹ is

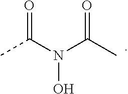

In certain embodiments, R¹ is

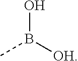

In certain embodiments, R¹ is

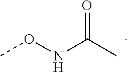

In certain embodiments, R¹ is

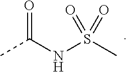

In certain embodiments, R¹ is

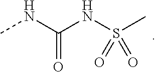

In certain embodiments, R¹ is

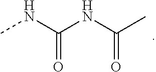

In certain embodiments, R¹ is

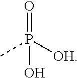

In certain embodiments, R¹ is

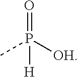

In certain embodiments, R¹ is

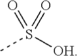

In certain embodiments, R¹ is

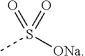

In certain embodiments, R¹ is

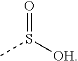

In certain embodiments, R¹ is

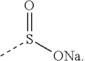

In certain embodiments, R¹ is

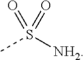

In certain embodiments,

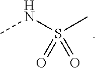

In certain embodiments, R¹ is

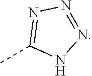

In certain embodiments, R¹ is

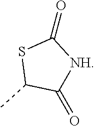

In certain embodiments, R¹ is

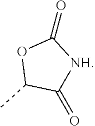

In certain embodiments, R¹ is

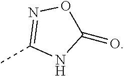

In certain embodiments, R¹ is

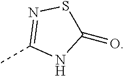

In certain embodiments, R¹ is

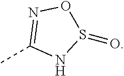

In certain embodiments, R¹ is

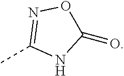

In certain embodiments, R¹ is

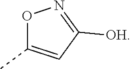

In certain embodiments, R¹ is

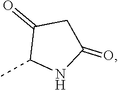

In certain embodiments, R¹ is

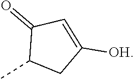

In certain embodiments, R¹ is

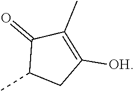

In certain embodiments, R¹ is

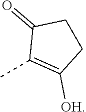

In certain embodiments, R¹ is

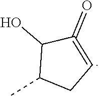

In certain embodiments, R¹ is

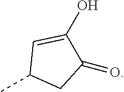

In certain embodiments, R is

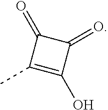

In certain embodiments, R¹ is

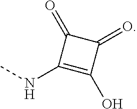

In certain embodiments, R¹ is

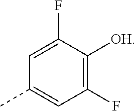

In certain embodiments, R¹ is

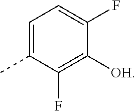

In certain embodiments, R¹ is

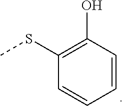

In certain embodiments, R¹ is

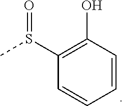

In certain embodiments, R¹ is.

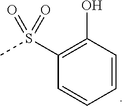

In certain embodiments, L¹ is a bond.

In certain embodiments, R^{4a} is CH₃. In certain embodiments, R^{4b} is CH₃.

In certain embodiments, R^{5a} is H. In certain embodiments, R^{5b} is H.

In certain embodiments, R^{3a} is H. In certain embodiments, R^{3a} is F. In certain embodiments, R^{3a} is Cl. In certain embodiments, R^{3a} is Br. In certain embodiments, R^{3a} is CH₃. In certain embodiments, R^{3a} is OCH₃. In certain embodiments, R^{3a} is N(CH₃)₂. In certain embodiments, R^{3b} is H. In certain embodiments, R^{3b} is F. In certain embodiments, R^{3b} is C₁. In certain embodiments, R^{3b} is Br. In certain embodiments, R^{3b} is CH₃. In certain embodiments, R^{3b} is OCH₃. In certain embodiments, R^{3b} is N(CH₃)₂. In certain embodiments, R^{3c} is H. In certain embodiments, R^{3c} is F. In certain embodiments, R^{3c} is Cl. In certain embodiments, R^{3c} is Br. In certain embodiments, R^{3c} is CH₃. In certain embodiments, R^{3c} is OCH₃. In certain embodiments, R^{3c} is N(CH₃)₂. In certain embodiments, R^{3d} is H. In certain embodiments, R^{3d} is F. In certain embodiments, R^{3d} is C₁. In certain embodiments, R^{3d} is Br. In certain embodiments, R^{3d} is CH₃. In certain embodiments, R^{3d} is OCH₃. In certain embodiments, R^{3d} is N(CH₃)₂.

In certain embodiments, at least three of R^{3a}, R^{3b}, R^{3c}, and R^{3d} are H.

In certain embodiments, R^{3b} is selected from the group consisting of F, Cl, Br, CH₃, OCH₃, and N(CH₃) and R^{3a}, R^{3c}, and R^{3d} are each H. In certain embodiments, R^{3c} is selected from the group consisting of F, Cl, Br, CH₃, OCH₃, and N(CH₃) and R^{3a}, R^{3b}, and R^{3d} are each H.

In certain embodiments, the compound of Formula (I) is selected from the group consisting of:
5,5-dimethyl-4,5-dihydronaphtho[1,2-b]thiophene-2-carboxamide;
5,5-dimethyl-4,5-dihydronaphtho[1,2-b]thiophene-2-carbonitrile;
5-(5,5-dimethyl-4,5-dihydronaphtho[1,2-b]thiophen-2-yl)-2H-tetrazole;
5,5-dimethyl-N-(methylsulfonyl)-4,5-dihydronaphtho[1,2-b]thiophene-2-carboxamide;
5,5-dimethyl-4-oxo-4,5-dihydronaphtho[1,2-b]thiophene-2-carboxylic acid; and
methyl 3-(5,5-dimethyl-4,5-dihydronaphtho[1,2-b]thiophen-2-yl)-2,2-dihydroxy-3-oxopropanoate.

In another aspect, the disclosure provides a compound of Formula (II) or a pharmaceutically acceptable salt thereof:

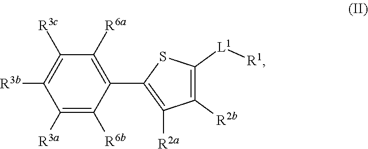

wherein:
   L¹ is selected from the group consisting of a bond and optionally substituted C₁-C₂ alkylenyl;
   R¹ is a carboxylic acid (—COOH), —C(═O)OR^{A}, —[C(═O)]₃R^{A}, —C(═O)C(OH)₂C(═O)OR^{A}, —C(═O)NHOR^{A}, H, a carboxylic acid bioisostere, or a carboxylic acid precursor;
   R^{2a} and R^{2b} are each independently H or optionally substituted C₁-C₆ alkyl;
   R^{3a}, R^{3b}, and R^{3c} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   R^{6a} and R^{6b} are each independently selected from the group consisting of H, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₆-C₁₀ aryl, optionally substituted C₂-C₈ heteroaryl, C(═O)R^{A}, and

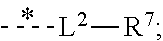

L² is selected from the group consisting of

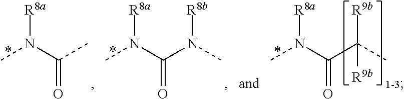

R⁷ is selected from the group consisting of

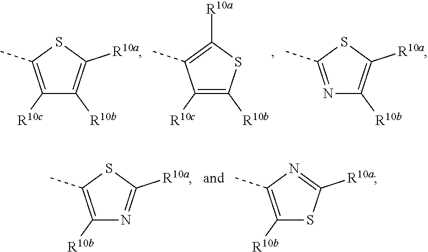

R^{8a} and R^{8b} are each independently selected from the group consisting of H, optionally substituted C₁-C₆ alkyl, and optionally substituted C₃-C₈ cycloalkyl, or R^{8a} and R^{8b} can combine with the atoms to which they are bound to form an optionally substituted C₂-C₈ heterocycloalkyl;
R^{9a} and R^{9b} are each independently selected from the group consisting of H and optionally substituted C₁-C₆ alkyl;
R^{10a}, R^{10b}, and R^{10c} are each independently selected from the group consisting of optionally substituted C₁-C₆ alkyl, optionally substituted C₃-C₈ cycloalkyl, optionally substituted C₆-C₁₀ aryl, optionally substituted C₆-C₈ heteroaryl, halogen, C(═O)R^{A}, C(═O)OR^{A}, C(═O)N(R^{A})(R^{B}), S(═O)R^{A}, S(═O)₂R^{A}, S(═O)N(R^{A})(R^{B}), and S(═O)₂N(R^{A})(R^{B}), or two vicinal substituents selected from the group consisting of R^{10a}, R^{10b}, and R^{10c} can combine with the atoms to which they are bound to form an optionally substituted C₃-C₈ cycloalkyl, optionally substituted C₂-C₈ heterocycloalkyl, optionally substituted C₆-C₁₀ aryl, or optionally substituted C₂-C₈ heteroaryl;
each occurrence of R^{A} and R^{B} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
wherein one of the following applies:
(a) R^{6a} and R^{6b} are each optionally substituted C₁-C₆ alkyl, wherein no more than one of R^{6a} and R^{6b} is optionally substituted C₁ alkyl;
(b) one of R^{6a} and R^{6b} is methyl, and one of R^{6a} and R^{6b} is selected from the group consisting of optionally substituted C₂-C₆ alkenyl, optionally substituted C₂-C₆ alkynyl, optionally substituted C₃-C₈ cycloalkyl, optionally substituted phenyl, and C₁-C₆ alkyl, wherein the optionally substituted alkyl is substituted with at least one optionally substituted phenyl;
(c) one of R^{6a} and R^{6b} is methyl, one of R^{6a} and R^{6b} is H, and one of R^{3a} and R^{3c} is selected from the group consisting of optionally substituted C₁-C₆ alkyl, optionally substituted C₂-C₆ alkenyl, optionally substituted C₂-C₆ alkynyl, optionally substituted C₃-C₈ cycloalkyl, optionally substituted phenyl, and C₁-C₆ alkyl; and
(d) one of R^{6a} and R^{6b} is H, and one of R^{6a} and R^{6b} is selected from the group consisting of C₁-C₆ alkyl, optionally substituted C₁-C₆ haloalkoxy, optionally substituted C₂-C₆ alkenyl, optionally substituted C₂-C₆ alkynyl, and C(═O)H, wherein the C₁-C₆ alkyl is substituted with at least one substituent selected from the group consisting of C₂-C₈ alkynyl, C₁-C₆ alkoxy, C═O, and CN;
(e) one of R^{6a} and R^{6b} is

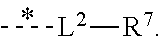

In certain embodiments, R¹ is selected from the group consisting of

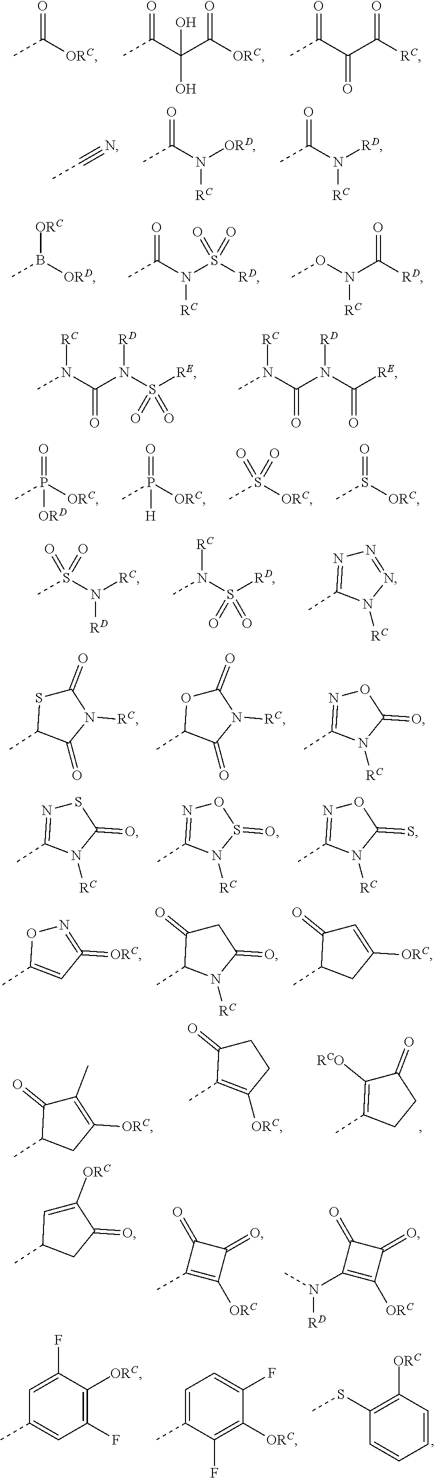

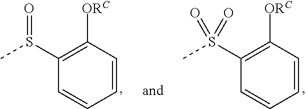

wherein R^{C}, R^{D}, and R^{E}, if present, are each independently selected from the group consisting of H, optionally substituted C₁-C₆ alkyl, and C(═O)(optionally substituted C₁-C₆ alkyl).

In certain embodiments, R¹ is

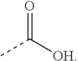

In certain embodiments, R¹ is

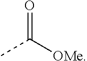

### In

certain embodiments, R¹ is

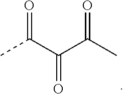

In certain embodiments, R¹ is

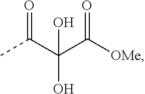

In certain embodiments, R¹ is

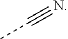

In certain embodiments, R¹ is

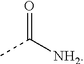

In certain embodiments, R¹ is

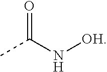

In certain embodiments, R¹ is

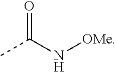

In certain embodiments, R¹ is

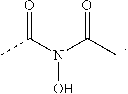

In certain embodiments, R¹ is

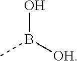

In certain embodiments, R¹ is

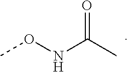

In certain embodiments, R¹ is

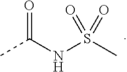

In certain embodiments, R¹ is

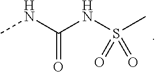

In certain embodiments, R¹ is

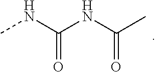

In certain embodiments, R¹ is

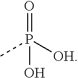

In certain embodiments, R¹ is

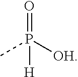

In certain embodiments, R¹ is

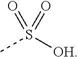

In certain embodiments, R¹ is

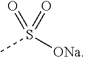

In certain embodiments, R¹ is

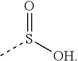

In certain embodiments, R¹ is

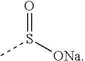

In certain embodiments, R¹ is

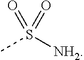

In certain embodiments, R¹ is

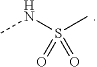

In certain embodiments, R¹ is

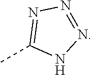

In certain embodiments, R¹ is

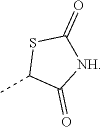

In certain embodiments, R¹ is

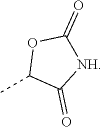

In certain embodiments, R¹ is

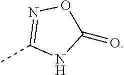

In certain embodiments, R¹ is

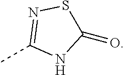

In certain embodiments, R¹ is

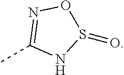

In certain embodiments, R¹ is

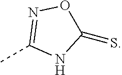

In certain embodiments, R¹ is

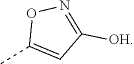

In certain embodiments, R¹ is

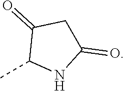

In certain embodiments, R¹ is

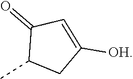

In certain embodiments, R¹ is

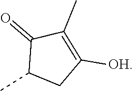

In certain embodiments, R¹ is

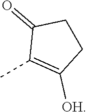

In certain embodiments, R¹ is

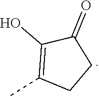

In certain embodiments, R¹ is

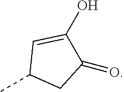

In certain embodiments, R¹ is

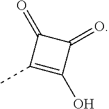

In certain embodiments, R¹ is

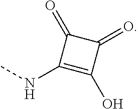

In certain embodiments, R¹

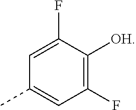

In certain embodiments, R¹ is

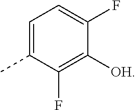

In certain embodiments, R¹

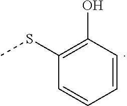

In certain embodiments, R¹ is

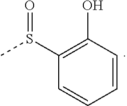

In certain embodiments, R¹ is

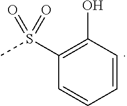

In certain embodiments, L¹ is a bond.

In certain embodiments, R^{3a} is H. In certain embodiments, R^{3a} is CH₃. In certain embodiments, R^{3a} is

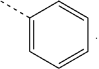

In certain embodiments, R^{3a} is

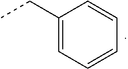

In certain embodiments, R^{3a} is

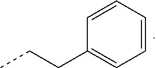

In certain embodiments, R^{3a} is

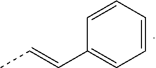

In certain embodiments, R^{3a} is

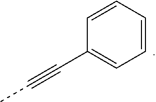

In certain embodiments, R^{3b} is H. In certain embodiments, R^{3b} is CH₃. In certain embodiments, R^{3b} is

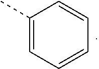

In certain embodiments, R^{3b} is

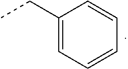

In certain embodiments, R^{3b} is

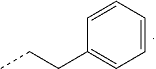

In certain embodiments, R^{3b} is

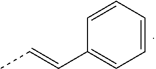

In certain embodiments, R^{3b} is

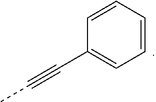

In certain embodiments, R^{3c} is H. In certain embodiments, R^{3c} is CH₃. In certain embodiments, R^{3c} is

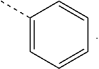

In certain embodiments, R^{3c} is

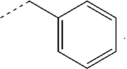

In certain embodiments, R^{3c} is

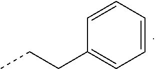

In certain embodiments, R^{3c} is

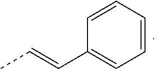

In certain embodiments, R^{3c} is

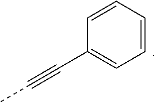

In certain embodiments, R^{6a} is ethyl and R^{6b} is methyl. In certain embodiments, R^{6a} is ethyl and R^{6b} is ethyl. In certain embodiments, R^{6a} is methyl and R^{6b} is ethyl.

In certain embodiments

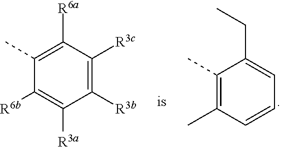

In certain embodiments,

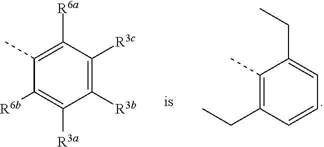

In certain embodiments,

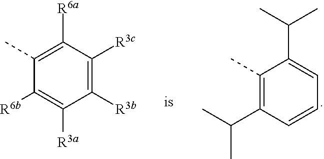

In certain embodiments,

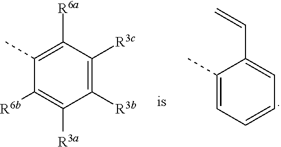

In certain embodiments,

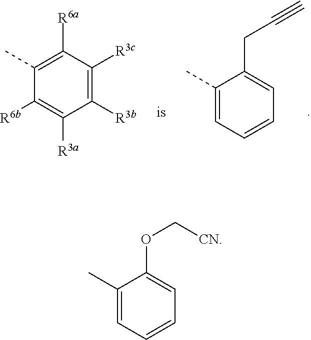

In certain embodiments,

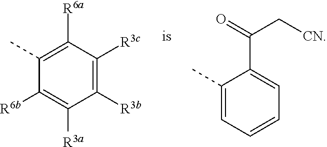

In certain embodiments,

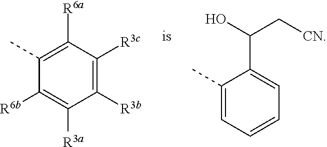

In certain embodiments,

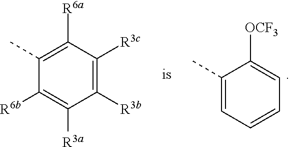

In certain embodiments,

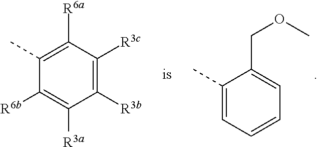

In certain embodiments,

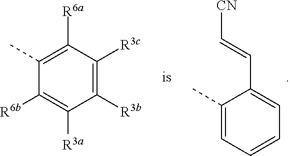

In certain embodiments,

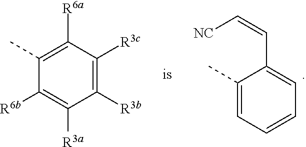

In certain embodiments,

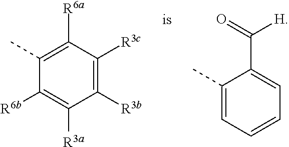

In certain embodiments R is

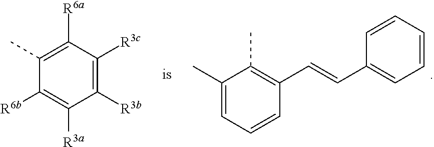

In certain embodiments,

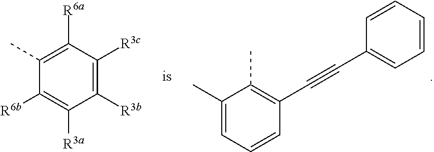

In certain embodiments,

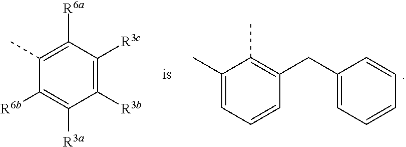

In certain embodiments,

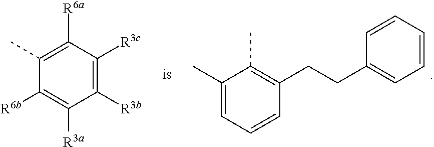

In certain embodiments,

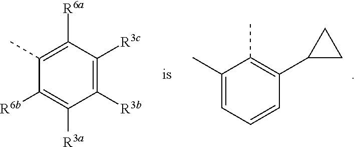

In certain embodiments,

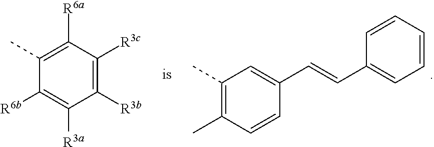

In certain embodiments,

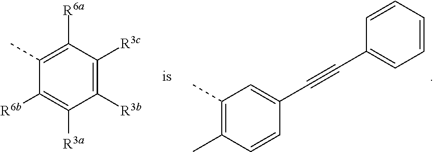

In certain embodiments,

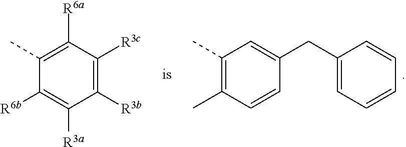

In certain embodiments

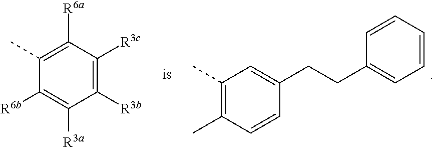

In certain embodiments,

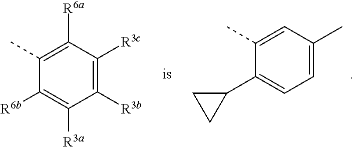

In certain embodiments,

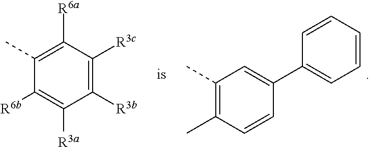

In certain embodiments,

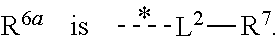

In certain embodiments, R^{6b} is H. In certain embodiments, CH₃. In certain embodiments, CF₃. In certain embodiments, CH═CH₂.

In certain embodiments,

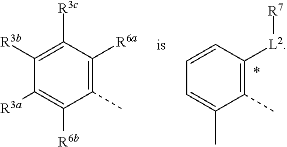

In certain embodiments,

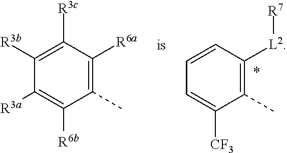

In certain embodiments,

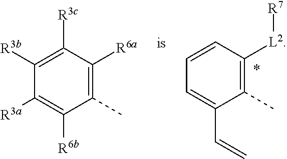

In certain embodiments,

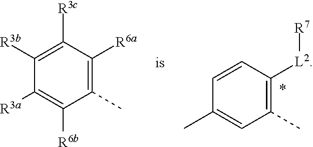

In certain embodiments,

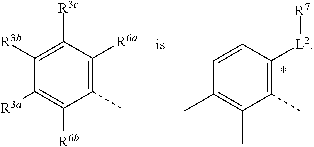

In certain embodiments,

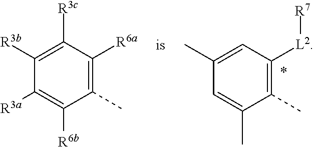

In certain embodiments, R^{8a} is H. In certain embodiments, R^{8a} is Me. In certain embodiments, R^{8a} is Et. In certain embodiments, R^{8a} is CH₂CN. In certain embodiments, R^{8a} is CH₂C≡CH. In certain embodiments, R^{8a} is iPr. In certain embodiments, R^{8a} is nPr. In certain embodiments, R^{8a} is nBu. In certain embodiments, R^{8a} is

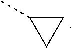

In certain embodiments, R^{8a} is

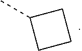

In certain embodiments, R^{8b} is H. In certain embodiments, R^{8b} is Me. In certain embodiments, R^{8a} is Et. In certain embodiments, R^{8b} is CH₂CN. In certain embodiments, R^{8b} is CH₂C≡CH. In certain embodiments, R^{8b} is iPr. In certain embodiments, R^{8b} is nPr. In certain embodiments, R^{8b} is nBu. In certain embodiments, R^{8b} is

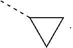

In certain embodiments, R^{8b} is

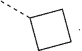

In certain embodiments, R^{8a} and R^{8b} combine with the atoms to which they are bound to form

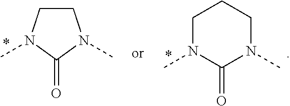

In certain embodiments, L² is

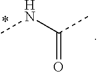

In certain embodiments, L² is

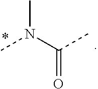

In certain embodiments, L² is

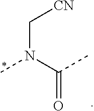

In certain embodiments, L²

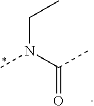

In certain embodiments, L² is

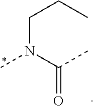

In certain embodiments, L² is

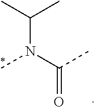

In certain embodiments, L² is

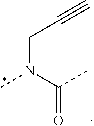

In certain embodiments, L² is

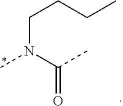

In certain embodiments, L² is

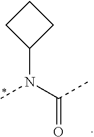

In certain embodiments, L² is

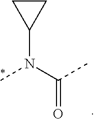

In certain embodiments, L² is

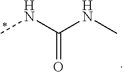

In certain embodiments, L² is

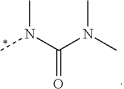

In certain embodiments, L² is

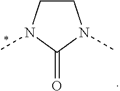

In certain embodiments, L² is

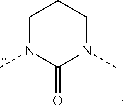

In certain embodiments, L² is

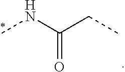

In certain embodiments, L² is

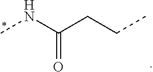

In certain embodiments, R^{10a} is H. In certain embodiments, R^{10a} is Me. In certain embodiments, R^{10a} is CF₂H. In certain embodiments, R^{10a} is CF₃. In certain embodiments, R^{10a} is Et. In certain embodiments, R^{10a} is tBu. In certain embodiments, R^{10a} is F. In certain embodiments, R^{10a} is Cl. In certain embodiments, R^{10a} is OMe. In certain embodiments, R^{10a} is CN. In certain embodiments, R^{10a} is C(═O)OH. In certain embodiments, R^{10a} is C(═O)OMe. In certain embodiments, R^{10a} is C(═O)NH₂. In certain embodiments, R^{10a} is C(═O)Me. In certain embodiments, R^{10a} is S(═O)₂Me. In certain embodiments, R^{10a} is

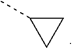

In certain embodiments, R^{10a} is

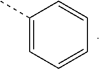

In certain embodiments, R^{10a} is

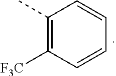

In certain embodiments, R^{10a} is

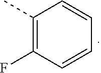

In certain embodiments, R^{10a} is

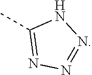

In certain embodiments, R^{10b} is H. In certain embodiments, R^{10b} is Me. In certain embodiments, R^{10b} is CF₂H. In certain embodiments, R^{10b} is CF₃. In certain embodiments, R^{10b} is Et. In certain embodiments, R^{10b} is tBu. In certain embodiments, R^{10b} is F. In certain embodiments, R^{10b} is Cl. In certain embodiments, R^{10b} is OMe. In certain embodiments, R^{10b} is CN. In certain embodiments, R^{10b} is C(═O)OH. In certain embodiments, R^{10b} is C(═O)OMe. In certain embodiments, R^{10b} is C(═O)NH₂. In certain embodiments, R^{10b} is C(═O)Me. In certain embodiments, R^{10b} is S(═O)₂Me. In certain embodiments, R^{10b} is

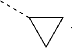

In certain embodiments, R^{10b} is

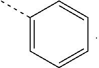

In certain embodiments, R^{10b} is

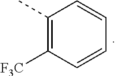

In certain embodiments, R^{10b} is

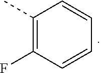

In certain embodiments, R^{10b} is

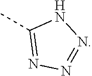

In certain embodiments, R^{10c} is H. In certain embodiments, R^{10c} is Me. In certain embodiments, R^{10c} is CF₂H. In certain embodiments, R^{10c} is CF₃. In certain embodiments, R^{10c} is Et. In certain embodiments, R^{10c} is tBu. In certain embodiments, R^{10c} is F. In certain embodiments, R^{10c} is C₁. In certain embodiments, R^{10c} is OMe. In certain embodiments, R^{10c} is CN. In certain embodiments, R^{10c} is C(═O)OH. In certain embodiments, R^{10c} is C(═O)OMe. In certain embodiments, R^{10c} is C(═O)NH₂. In certain embodiments, R^{10c} is C(═O)Me. In certain embodiments, R^{10c} is S(═O)₂Me. In certain embodiments, R^{10c} is

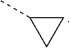

In certain embodiments, R^{10c} is

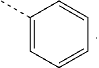

In certain embodiments, R^{10c} is

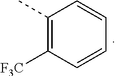

In certain embodiments, R^{10c} is

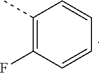

In certain embodiments, R^{10c} is

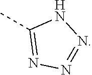

In certain embodiments, R⁷ is

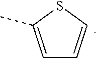

In certain embodiments, R⁷ is

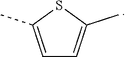

In certain embodiments, R⁷ is

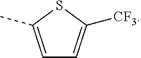

In certain embodiments, R⁷ is

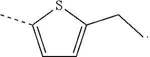

In certain embodiments, R⁷ is

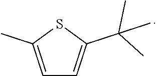

In certain embodiments, R⁷ is

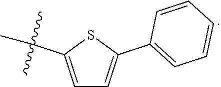 [SMILES: *c1ccc(-c2ccccc2)s1]

certain embodiments, R⁷ is

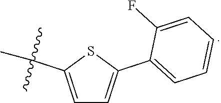 [SMILES: *c1ccc(-c2ccccc2F)s1]

In certain embodiments, R⁷ is

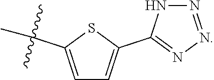 [SMILES: *c1ccc(-c2nnn[nH]2)s1]

In certain embodiments, R⁷ is

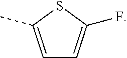

In certain embodiments, R⁷ is

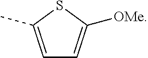

In certain embodiments, R⁷ is

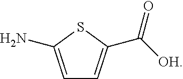

In certain embodiments R⁷ is

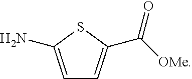

In certain embodiments, R⁷ is

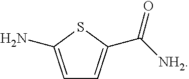

In certain embodiments, R⁷ is

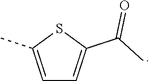

In certain embodiments, R⁷ is

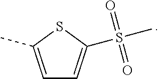

In certain embodiments, R⁷ is

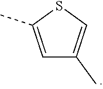

In certain embodiments, R⁷ is

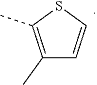

In certain embodiments, R⁷ is

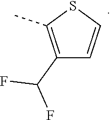

In certain embodiments, R⁷

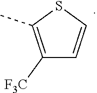

In certain embodiments, R⁷ is

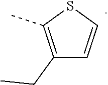

In certain embodiments, R⁷ is

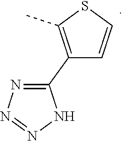

In certain embodiments, R⁷ is

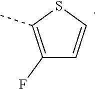

In certain embodiments, R⁷ is

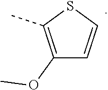

In certain embodiments, R⁷ is

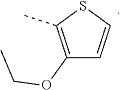

In certain embodiments, R⁷ is

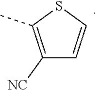

In certain embodiments, R⁷ is

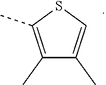

In certain embodiments, R⁷ is

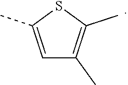

In certain embodiments, R⁷ is

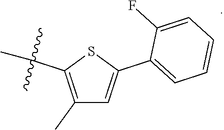 [SMILES: *c1sc(-c2ccccc2F)cc1C]

In certain embodiments, R⁷ is

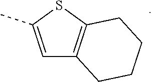

In certain embodiments, R⁷ is

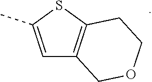

In certain embodiments, R⁷ is

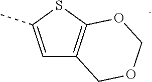

In certain embodiments, R⁷ is

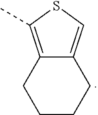

In certain embodiments, R⁷ is

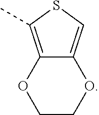

In certain embodiments, R⁷ is

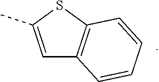

In certain embodiments, R⁷ is

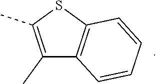

In certain embodiments, R⁷ is

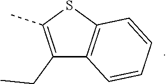

In certain embodiments, R⁷ is

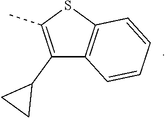

In certain embodiments, R⁷ is

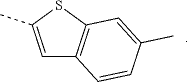

In certain embodiments, R⁷ is

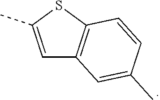

In certain embodiments, R⁷ is

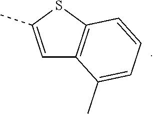

certain embodiments, R⁷ is

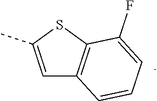

In certain embodiments, R⁷ is

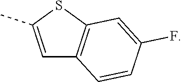

In certain embodiments, R⁷ is

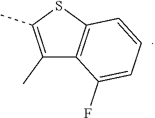

In certain embodiments, R⁷ is

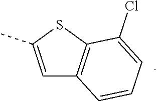

In certain embodiments, R⁷ is

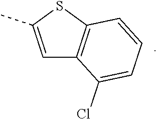

In certain embodiments, R⁷ is

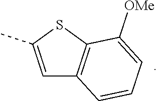

In certain embodiments, R⁷ is

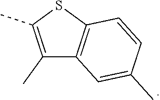

In certain embodiments, R⁷ is

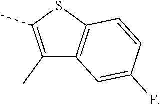

In certain embodiments, R⁷ is

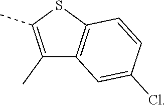

In certain embodiments, R⁷ is

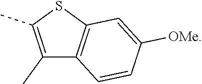

In certain embodiments, R⁷ is

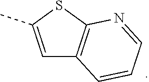

In certain embodiments, R⁷ is

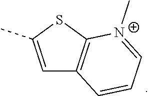

In certain embodiments, R⁷ is

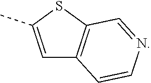

In certain embodiments, R⁷ is

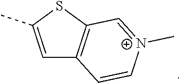

In certain embodiments, R⁷ is

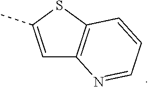

In certain embodiments, R⁷ is

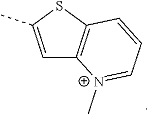

In certain embodiments, R⁷ is

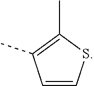

In certain embodiments, R⁷ is

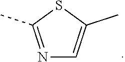

In certain embodiments, R⁷ is

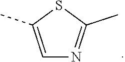

In certain embodiments, R⁷ is

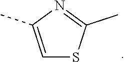

In certain embodiments, the compound of Formula (II) is selected from the group consisting of:
5-(2-ethyl-6-methylphenyl)thiophene-2-carboxylic acid;
5-(2,6-diethylphenyl)thiophene-2-carboxylic acid;
5-(2,6-diisopropylphenyl)thiophene-2-carboxylic acid;
5-(2-vinylphenyl)thiophene-2-carboxylic acid;
5-(2-(2-cyano-1-hydroxyethyl)phenyl)thiophene-2-carboxylic acid;
5-(2-(prop-2-yn-1-yl)phenyl)thiophene-2-carboxylic acid;
5-(2-(trifluoromethoxy)phenyl)thiophene-2-carboxylic acid;
5-(2-(methoxymethyl)phenyl)thiophene-2-carboxylic acid;
5-(2-(2-cyanoacetyl)phenyl)thiophene-2-carboxylic acid;
5-(2-(cyanomethoxy)phenyl)thiophene-2-carboxylic acid;
1-(5-(2,6-diethylphenyl)thiophen-2-yl)butane-1,2,3-trione;
(E)-5-(2-(2-cyanovinyl)phenyl)thiophene-2-carboxylic acid;
(Z)-5-(2-(2-cyanovinyl)phenyl)thiophene-2-carboxylic acid;
5-(2-formylphenyl)thiophene-2-carboxylic acid;
5-(2,6-diethylphenyl)-N-hydroxythiophene-2-carboxamide;
5-(2,6-diethylphenyl)-N-methoxythiophene-2-carboxamide;
5-(2-methyl-5-(phenylethynyl)phenyl)thiophene-2-carboxylic acid;
(E)-5-(2-methyl-5-styrylphenyl)thiophene-2-carboxylic acid;
5-(2-methyl-5-phenethylphenyl)thiophene-2-carboxylic acid;
5-(5-benzyl-2-methylphenyl)thiophene-2-carboxylic acid;
5-(4-methyl-[1,1′-biphenyl]-3-yl)thiophene-2-carboxylic acid;
5-(5-cyclopropyl-2-methylphenyl)thiophene-2-carboxylic acid;
5-(2-benzyl-6-methylphenyl)thiophene-2-carboxylic acid;
5-(2-methyl-6-(phenylethynyl)phenyl)thiophene-2-carboxylic acid;
(E)-5-(2-methyl-6-styrylphenyl)thiophene-2-carboxylic acid;
5-(2-methyl-6-phenethylphenyl)thiophene-2-carboxylic acid;
5-(2-cyclopropyl-5-methylphenyl)thiophene-2-carboxylic acid;
5-(2-(N,5-dimethylthiophene-2-carboxamido)-6-methylphenyl)thiophene-2-carboxylic acid;
5-(2-(benzo[b]thiophene-2-carboxamido)-6-methylphenyl)thiophene-2-carboxylic acid;
5-(2-methyl-6-(6-methylbenzo[b]thiophene-2-carboxamido)phenyl)thiophene-2-carboxylic acid;
5-(2-methyl-6-(N-methylbenzo[b]thiophene-2-carboxamido)phenyl)thiophene-2-carboxylic acid;
5-(2-(N,6-dimethylbenzo[b]thiophene-2-carboxamido)-6-methylphenyl)thiophene-2-carboxylic acid;
5-(2-methyl-6-(2-(thiophen-2-yl)acetamido)phenyl)thiophene-2-carboxylic acid;
5-(2-methyl-6-(5-methylfuran-2-carboxamido)phenyl)thiophene-2-carboxylic acid;
5-(2-methyl-6-(3-methylthiophene-2-carboxamido)phenyl)thiophene-2-carboxylic acid;
5-(2-(N,3-dimethylthiophene-2-carboxamido)-6-methylphenyl)thiophene-2-carboxylic acid;
5-(2-methyl-6-(5-methylthiazole-2-carboxamido)phenyl)thiophene-2-carboxylic acid;
5-(2-(N,5-dimethylthiazole-2-carboxamido)-6-methylphenyl)thiophene-2-carboxylic acid;
5-(2-methyl-6-(4-methylthiophene-2-carboxamido)phenyl)thiophene-2-carboxylic acid;
5-(2-(N-butyl-5-methylthiophene-2-carboxamido)-6-methylphenyl)thiophene-2-carboxylic acid;
5-(2-methyl-6-(2-methylthiazole-4-carboxamido)phenyl)thiophene-2-carboxylic acid;
5-(2-methyl-6-(2-methylthiazole-5-carboxamido)phenyl)thiophene-2-carboxylic acid;
5-(2-methyl-6-(2-methylthiophene-3-carboxamido)phenyl)thiophene-2-carboxylic acid;
5-(2-(N,2-dimethylthiazole-4-carboxamido)-6-methylphenyl)thiophene-2-carboxylic acid;
5-(2-methyl-6-(thiophene-2-carboxamido)phenyl)thiophene-2-carboxylic acid5-(2-methyl-6-(thiophene-2-carboxamido)phenyl)thiophene-2-carboxylic acid;
5-(2-methyl-6-(N-methylthiophene-2-carboxamido)phenyl)thiophene-2-carboxylic acid;
5-(2-(N-ethyl-5-methylthiophene-2-carboxamido)-6-methylphenyl)thiophene-2-carboxylic acid;
5-(2-methyl-6-(3-(5-methylthiophen-2-yl)ureido)phenyl)thiophene-2-carboxylic acid5-(2-methyl-6-(3-(5-methylthiophen-2-yl)ureido)phenyl)thiophene-2-carboxylic acid;
5-(2-methyl-6-(5-methyl-N-propylthiophene-2-carboxamido)phenyl)thiophene-2-carboxylic acid;
5-(2-(N-cyclobutyl-5-methylthiophene-2-carboxamido)-6-methylphenyl)thiophene-2-carboxylic acid;
5-(2-(N,5-dimethylthiophene-2-carboxamido)-6-vinylphenyl)thiophene-2-carboxylic acid;
5-(2-(N,5-dimethylthiophene-2-carboxamido)-6-ethylphenyl)thiophene-2-carboxylic acid;
5-(2-methyl-6-(3-(5-methylthiophen-2-yl)-2-oxoimidazolidin-1-yl)phenyl)thiophene-2-carboxylic acid;
5-(2-(5-ethylthiophene-2-carboxamido)-6-methylphenyl)thiophene-2-carboxylic acid5-(2-(5-ethylthiophene-2-carboxamido)-6-methylphenyl)thiophene-2-carboxylic acid;
5-(2-(5-ethyl-N-methylthiophene-2-carboxamido)-6-methylphenyl)thiophene-2-carboxylic acid;
5-(2-(5-methoxythiophene-2-carboxamido)-6-methylphenyl)thiophene-2-carboxylic acid5-(2-(5-methoxythiophene-2-carboxamido)-6-methylphenyl)thiophene-2-carboxylic acid;
5-(2-(5-methoxy-N-methylthiophene-2-carboxamido)-6-methylphenyl)thiophene-2-carboxylic acid;
5-((2-(5-carboxythiophen-2-yl)-3-methylphenyl)carbamoyl)thiophene-2-carboxylic acid;
5-(2-(5-carboxy-N-methylthiophene-2-carboxamido)-6-methylphenyl)thiophene-2-carboxylic acid;
5-(2-(5-(tert-butyl)thiophene-2-carboxamido)-6-methylphenyl)thiophene-2-carboxylic acid;
5-(2-(5-fluorothiophene-2-carboxamido)-6-methylphenyl)thiophene-2-carboxylic acid5-(2-(5-fluorothiophene-2-carboxamido)-6-methylphenyl)thiophene-2-carboxylic acid;
5-(2-methyl-6-(4-methylbenzo[b]thiophene-2-carboxamido)phenyl)thiophene-2-carboxylic acid;
5-(2-(4,5-dimethylthiophene-2-carboxamido)-6-methylphenyl)thiophene-2-carboxylic acid;
5-(2-(6,7-dihydro-4H-thieno[3,2-c]pyran-2-carboxamido)-6-methylphenyl)thiophene-2-carboxylic acid5-(2-(6,7-dihydro-4H-thieno[3,2-c]pyran-2-carboxamido)-6-methylphenyl)thiophene-2-carboxylic acid;
5-(2-methyl-6-(3-methylbenzo[b]thiophene-2-carboxamido)phenyl)thiophene-2-carboxylic acid;
5-(2-methyl-6-(5-(trifluoromethyl)thiophene-2-carboxamido)phenyl)thiophene-2-carboxylic acid;
5-(2-methyl-6-(4,5,6,7-tetrahydrobenzo[b]thiophene-2-carboxamido)phenyl)thiophene-2-carboxylic acid;
5-(2-methyl-6-(N,4,5-trimethylthiophene-2-carboxamido)phenyl)thiophene-2-carboxylic acid5-(2-methyl-6-(N,4,5-trimethylthiophene-2-carboxamido)phenyl)thiophene-2-carboxylic acid;
5-(2-(5-fluoro-N-methylthiophene-2-carboxamido)-6-methylphenyl)thiophene-2-carboxylic acid;
5-(2-(N,4-dimethylbenzo[b]thiophene-2-carboxamido)-6-methylphenyl)thiophene-2-carboxylic acid;
5-(2-methyl-6-(N,3,4-trimethylthiophene-2-carboxamido)phenyl)thiophene-2-carboxylic acid;
5-(2-methyl-6-(N-methyl-4,5,6,7-tetrahydrobenzo[b]thiophene-2-carboxamido)phenyl)thiophene-2-carboxylic acid;
5-(2-methyl-6-(3-methyl-N-propylthiophene-2-carboxamido)phenyl)thiophene-2-carboxylic acid;
5-(2-(N,3-dimethylbenzo[b]thiophene-2-carboxamido)-6-methylphenyl)thiophene-2-carboxylic acid;
5-(2-methyl-6-(N-methyl-2,3-dihydrothieno[2,3-b][1,4]dioxine-6-carboxamido)phenyl)thiophene-2-carboxylic acid;
5-(2-(N,5-dimethylbenzo[b]thiophene-2-carboxamido)-6-methylphenyl)thiophene-2-carboxylic acid;
5-(2-(3,4-dimethylthiophene-2-carboxamido)-6-methylphenyl)thiophene-2-carboxylic acid;
5-(2-methyl-6-(5-methylbenzo[b]thiophene-2-carboxamido)phenyl)thiophene-2-carboxylic acid;
5-(2-(5-acetylthiophene-2-carboxamido)-6-methylphenyl)thiophene-2-carboxylic acid;
5-(2-methyl-6-(4,5,6,7-tetrahydrobenzo[c]thiophene-1-carboxamido)phenyl)thiophene-2-carboxylic acid;
5-(2-methyl-6-(N-methyl-4,5,6,7-tetrahydrobenzo[c]thiophene-1-carboxamido)phenyl)thiophene-2-carboxylic acid;
5-(2-(N-(cyanomethyl)-5-methylthiophene-2-carboxamido)-6-methylphenyl)thiophene-2-carboxylic acid;
5-(2-methyl-6-(5-methyl-N-(prop-2-yn-1-yl)thiophene-2-carboxamido)phenyl)thiophene-2-carboxylic acid;
5-(2-(N-isopropyl-5-methylthiophene-2-carboxamido)-6-methylphenyl)thiophene-2-carboxylic acid
5-(2-(N-cyclopropyl-5-methylthiophene-2-carboxamido)-6-methylphenyl)thiophene-2-carboxylic acid;
5-(2-(5-methylthiophene-2-carboxamido)-6-(trifluoromethyl)phenyl)thiophene-2-carboxylic acid;
5-(2-(N,5-dimethylthiophene-2-carboxamido)-6-(trifluoromethyl)phenyl)thiophene-2-carboxylic acid;
N-(2-(5-(1H-tetrazol-5-yl)thiophen-2-yl)-3-methylphenyl)-N,5-dimethylthiophene-2-carboxamide;
N-(2-(5-(1H-tetrazol-5-yl)thiophen-2-yl)-3-methylphenyl)-N,3-dimethylthiophene-2-carboxamide;
5-((3-methyl-2-(thiophen-2-yl)phenyl)carbamoyl)thiophene-2-carboxylic acid;
5-(methyl(3-methyl-2-(thiophen-2-yl)phenyl)carbamoyl)thiophene-2-carboxylic acid;
5-((2-(5-carbamoylthiophen-2-yl)-3-methylphenyl)(methyl)carbamoyl)thiophene-2-carboxylic acid;
methyl 5-((2-(5-(1H-tetrazol-5-yl)thiophen-2-yl)-3-methylphenyl)(methyl)carbamoyl)thiophene-2-carboxylate;
5-((2-(5-(1H-tetrazol-5-yl)thiophen-2-yl)-3-methylphenyl)(methyl)carbamoyl)thiophene-2-carboxylic acid;
methyl 5-((2-(5-(methoxycarbonyl)thiophen-2-yl)-3-methylphenyl)carbamoyl)thiophene-2-carboxylate;
N-(2-(5-(1H-tetrazol-5-yl)thiophen-2-yl)-3-methylphenyl)-N,3-dimethylbenzo[b]thiophene-2-carboxamide;
5-(2-(5-carbamoyl-N-methylthiophene-2-carboxamido)-6-methylphenyl)thiophene-2-carboxylic acid;
5-(2-methyl-6-(N-methyl-5-(1H-tetrazol-5-yl)thiophene-2-carboxamido)phenyl)thiophene-2-carboxylic acid;
5-(2-(5-acetyl-N-methylthiophene-2-carboxamido)-6-methylphenyl)thiophene-2-carboxylic acid;
5-(2-methyl-6-(N-methyl-5-(trifluoromethyl)thiophene-2-carboxamido)phenyl)thiophene-2-carboxylic acid;
5-(2-(3-ethylbenzo[b]thiophene-2-carboxamido)-6-methylphenyl)thiophene-2-carboxylic acid;
5-(2-methyl-6-(3-(trifluoromethyl)thiophene-2-carboxamido)phenyl)thiophene-2-carboxylic acid;
5-(2-(5-carboxy-N-methylthiophene-2-carboxamido)-4,6-dimethylphenyl)thiophene-2-carboxylic acid;
5-(2-(3-fluorothiophene-2-carboxamido)-6-methylphenyl)thiophene-2-carboxylic acid;
5-(2-(3-cyclopropylbenzo[b]thiophene-2-carboxamido)-6-methylphenyl)thiophene-2-carboxylic acid;
5-(2-(3-methoxy-N-methylthiophene-2-carboxamido)-6-methylphenyl)thiophene-2-carboxylic acid;
5-(2-methyl-6-(N-methyl-2,3-dihydrothieno[3,4-b][1,4]dioxine-5-carboxamido)phenyl)thiophene-2-carboxylic acid;
5-(2-(3-ethyl-N-methylthiophene-2-carboxamido)-6-methylphenyl)thiophene-2-carboxylic acid;
5-(2-(3-ethoxy-N-methylthiophene-2-carboxamido)-6-methylphenyl)thiophene-2-carboxylic acid;
5-(2-(3-ethoxythiophene-2-carboxamido)-6-methylphenyl)thiophene-2-carboxylic acid;
methyl 5-(2-methyl-6-(N-methyl-3-(1H-tetrazol-5-yl)thiophene-2-carboxamido)phenyl)thiophene-2-carboxylate;
5-(2-methyl-6-(N-methyl-3-(1H-tetrazol-5-yl)thiophene-2-carboxamido)phenyl)thiophene-2-carboxylic acid;
5-(2-(5-fluoro-3-methylbenzo[b]thiophene-2-carboxamido)-6-methylphenyl)thiophene-2-carboxylic acid;
5-(2-(5-fluoro-N,3-dimethylbenzo[b]thiophene-2-carboxamido)-6-methylphenyl)thiophene-2-carboxylic acid;
5-(2-(3-ethyl-N-methylbenzo[b]thiophene-2-carboxamido)-6-methylphenyl)thiophene-2-carboxylic acid;
5-(2-(5-chloro-N,3-dimethylbenzo[b]thiophene-2-carboxamido)-6-methylphenyl)thiophene-2-carboxylic acid;
5-(2-(3-cyclopropyl-N-methylbenzo[b]thiophene-2-carboxamido)-6-methylphenyl)thiophene-2-carboxylic acid;
5-(2-(3-ethylthiophene-2-carboxamido)-6-methylphenyl)thiophene-2-carboxylic acid;
5-(2-(3-(difluoromethyl)thiophene-2-carboxamido)-6-methylphenyl)thiophene-2-carboxylic acid;
5-(2-(3-(difluoromethyl)-N-methylthiophene-2-carboxamido)-6-methylphenyl)thiophene-2-carboxylic acid;
5-(2-methyl-6-(thieno[2,3-b]pyridine-2-carboxamido)phenyl)thiophene-2-carboxylic acid;
5-(2-methyl-6-(N-methylthieno[2,3-b]pyridine-2-carboxamido)phenyl)thiophene-2-carboxylic acid;
2-((2-(5-carboxythiophen-2-yl)-3-methylphenyl)(methyl)carbamoyl)-7-methylthieno[2,3-b]pyridin-7-ium;
5-(2-methyl-6-(thieno[3,2-b]pyridine-2-carboxamido)phenyl)thiophene-2-carboxylic acid;
5-(2-methyl-6-(N-methylthieno[3,2-b]pyridine-2-carboxamido)phenyl)thiophene-2-carboxylic acid;
2-((2-(5-carboxythiophen-2-yl)-3-methylphenyl)(methyl)carbamoyl)-4-methylthieno[3,2-b]pyridin-4-ium;
5-(2-(7-fluorobenzo[b]thiophene-2-carboxamido)-6-methylphenyl)thiophene-2-carboxylic acid;
5-(2-(7-fluoro-N-methylbenzo[b]thiophene-2-carboxamido)-6-methylphenyl)thiophene-2-carboxylic acid;
5-(2-(3,5-dimethylbenzo[b]thiophene-2-carboxamido)-6-methylphenyl)thiophene-2-carboxylic acid;
5-(2-methyl-6-(N,3, 5-trimethylbenzo[b]thiophene-2-carboxamido)phenyl)thiophene-2-carboxylic acid;
5-(2-methyl-6-(5-phenylthiophene-2-carboxamido)phenyl)thiophene-2-carboxylic acid;
5-(2-methyl-6-(N-methyl-5-phenylthiophene-2-carboxamido)phenyl)thiophene-2-carboxylic acid;
2-((2-(5-carboxythiophen-2-yl)-3-methylphenyl)(methyl)carbamoyl)-6-methylthieno[2,3-c]pyridin-6-ium;
5-(2-(N,3-dimethylthiophene-2-carboxamido)-5-methylphenyl)thiophene-2-carboxylic acid;
5-(2-(N,3-dimethylthiophene-2-carboxamido)-5-ethylphenyl)thiophene-2-carboxylic acid;
5-(2-methyl-6-(N-methyl-5-(methylsulfonyl)thiophene-2-carboxamido)phenyl)thiophene-2-carboxylic acid;
5-(2-(5-(2-fluorophenyl)-N-methylthiophene-2-carboxamido)-6-methylphenyl)thiophene-2-carboxylic acid;
5-(2-(4-fluoro-N-methylbenzo[b]thiophene-2-carboxamido)-6-methylphenyl)thiophene-2-carboxylic acid;
5-(2-(7-chloro-N-methylbenzo[b]thiophene-2-carboxamido)-6-methylphenyl)thiophene-2-carboxylic acid;
5-(2-(4-chloro-N-methylbenzo[b]thiophene-2-carboxamido)-6-methylphenyl)thiophene-2-carboxylic acid;
5-(2-(6-fluoro-N-methylbenzo[b]thiophene-2-carboxamido)-6-methylphenyl)thiophene-2-carboxylic acid;
5-(2-(5-methoxy-N,3-dimethylbenzo[b]thiophene-2-carboxamido)-6-methylphenyl)thiophene-2-carboxylic acid;
5-(2-methyl-6-(N-methylthieno[2,3-c]pyridine-2-carboxamido)phenyl)thiophene-2-carboxylic acid;
5-(6-(N,3-dimethylbenzo[b]thiophene-2-carboxamido)-2,3-dimethylphenyl)thiophene-2-carboxylic acid;
5-(2-(7-methoxy-N-methylbenzo[b]thiophene-2-carboxamido)-6-methylphenyl)thiophene-2-carboxylic acid; and
5-(2-(N,3-dimethyl-5-(2-(trifluoromethyl)phenyl)thiophene-2-carboxamido)-6-methylphenyl)thiophene-2-carboxylic acid.

In another aspect, the disclosure provides a compound of Formula (III), or pharmaceutically acceptable salt thereof:

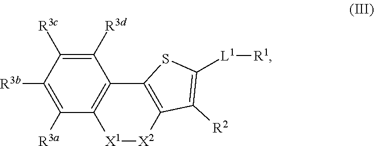

wherein:
   R¹ is a carboxylic acid (—COOH), a carboxylic acid bioisostere, or a carboxylic acid precursor;
   R² isH;
   R^{3a}, R^{3b}, R^{3b}, and R^{3d} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   L¹ is selected from the group consisting of a bond and optionally substituted C₁-C₂ alkylenyl;
   X¹ is selected from the group consisting of —C(R^{4a})(R^{4b})—, O, and S;
   X² is selected from the group consisting of —C(R^{5a})(R^{5b})— and a bond;
   R^{4a} and R^{4b} are each independently selected from the group consisting of H and optionally substituted C₁-C₆ alkyl,
      wherein no more than one of R^{4a} and R^{4b} is H;
   R^{5a} and R^{5b} are each independently selected from the group consisting of H and optionally substituted C₁-C₆ alkyl; and
   R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl.

In certain embodiments, the compound is selected from the group consisting of:
5-(4-fluoro-2,6-dimethylphenyl)thiophene-2-carboxylic acid;
5-(2-bromophenyl)thiophene-2-carboxylic acid;
5-(2-cyclohexylphenyl)thiophene-2-carboxylic acid;
5-(2-hydroxyphenyl)thiophene-2-carboxylic acid;
5-(2,3-dimethylphenyl)thiophene-2-carboxylic acid;
5-(2-methoxy-6-methylphenyl)-3-methylthiophene-2-carboxylic acid;
5-(4-amino-2,6-dimethylphenyl)thiophene-2-carboxylic acid;
5-(2-ethyl-6-(5-methylthiophene-2-carbonyl)phenyl)thiophene-2-carboxylic acid;
5-(2-ethyl-6-((5-methylthiophen-2-yl)methyl)phenyl)thiophene-2-carboxylic acid;
5,5-dimethyl-4-oxo-4,5-dihydronaphtho[1,2-b]thiophene-2-carboxylic acid;
5-(2-ethylphenyl)-4-methylthiophene-2-carboxylic acid;
5-(2-ethylphenyl)-1-methyl-1H-pyrazole-3-carboxylic acid;
5-(2-ethylphenyl)-1H-pyrazole-3-carboxylic acid; methyl 3-(5,5-dimethyl-4,5-dihydronaphtho[1,2-b]thiophen-2-yl)-2,2-dihydroxy-3-oxopropanoate;
methyl 5-(5-bromo-2-methylphenyl)thiophene-2-carboxylate;
1-(4-methyl-3-nitropyridin-2-yl)-4-((4-methylthiazol-2-yl)methyl)piperidin-4-ol;
5-(2-bromo-6-methylphenyl)thiophene-2-carboxylic acid; and methyl 5-(2-bromo-6-methylphenyl)thiophene-2-carboxylate.

In another aspect, the present disclosure provides a pharmaceutical composition comprising at least one compound of the present disclosure and a pharmaceutically acceptable carrier.

In certain embodiments, the pharmaceutical composition further comprises at least one additional therapeutically effective agent.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C ¹³C, ¹⁴C ³⁶Cl, ¹⁸F, ¹²³I, ¹²⁵I, ¹³N, ¹⁵N, ¹⁵O, ¹⁷O ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), allyloxycarbonyl (Alloc), methyl, ethyl, t-butyl, t-butyldimethylsilyl (TBDMS), 2-(trimethylsilyl)ethoxycarbonyl (Teoc), t-butyloxycarbonyl (Boc), para-methoxybenzyl (PMB), triphenylmethyl (trityl), acetyl, and fluorenylmethoxycarbonyl (FMOC). Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

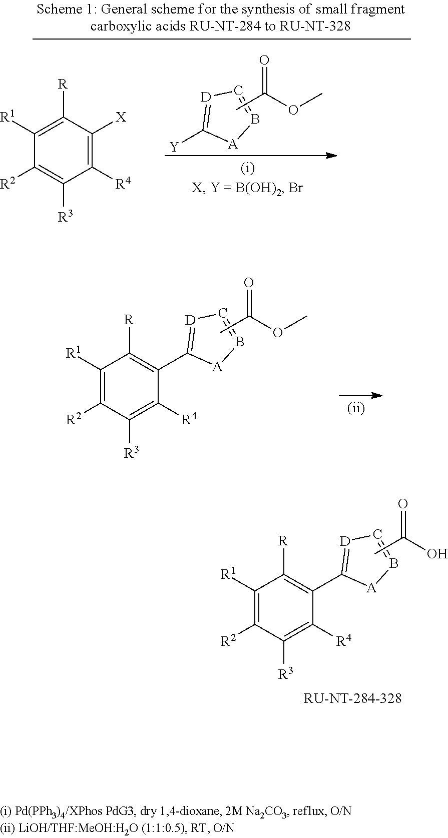

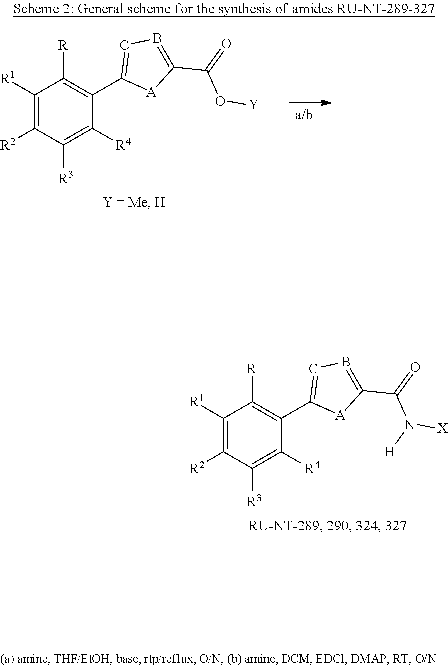

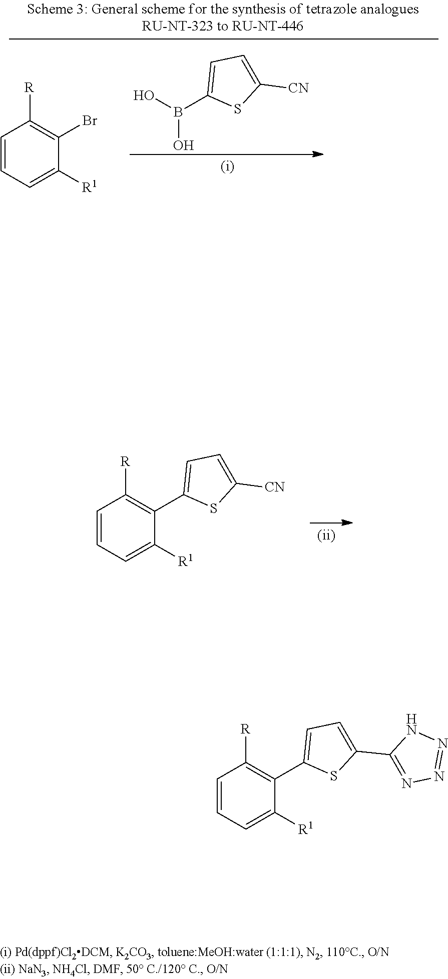

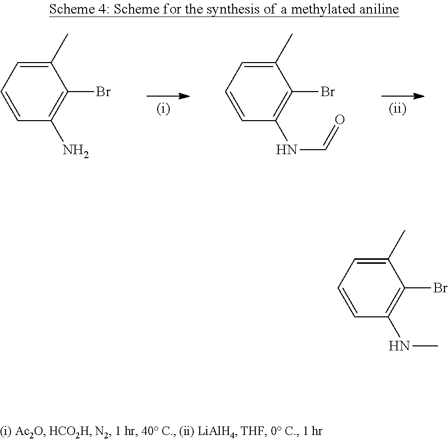

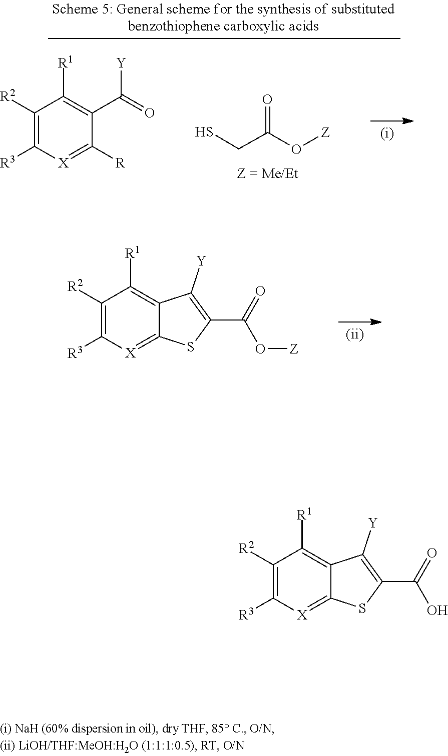

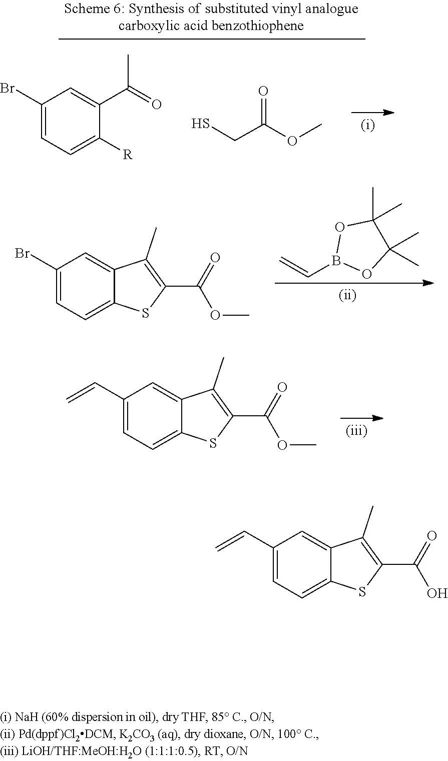

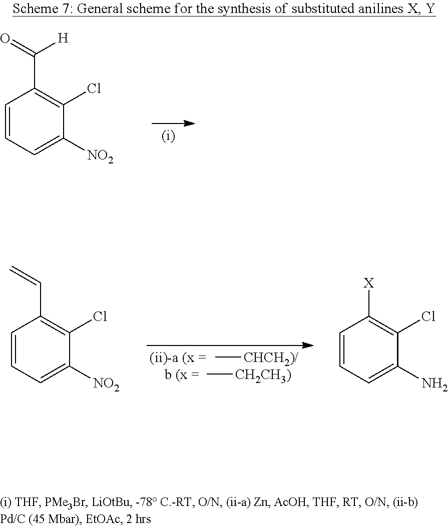

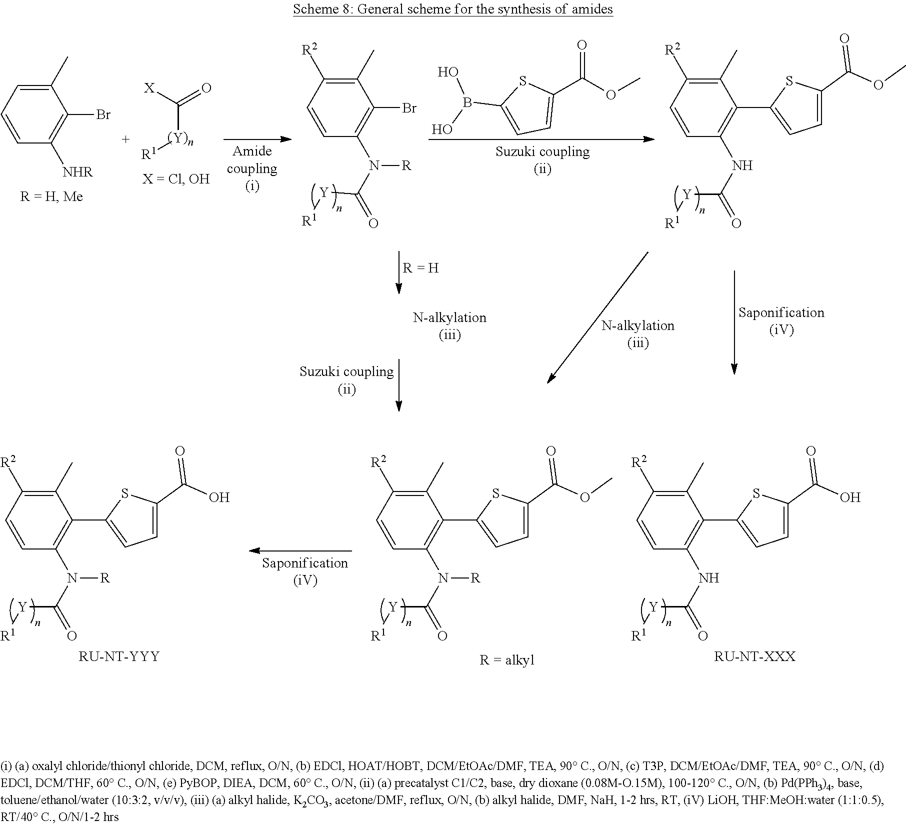

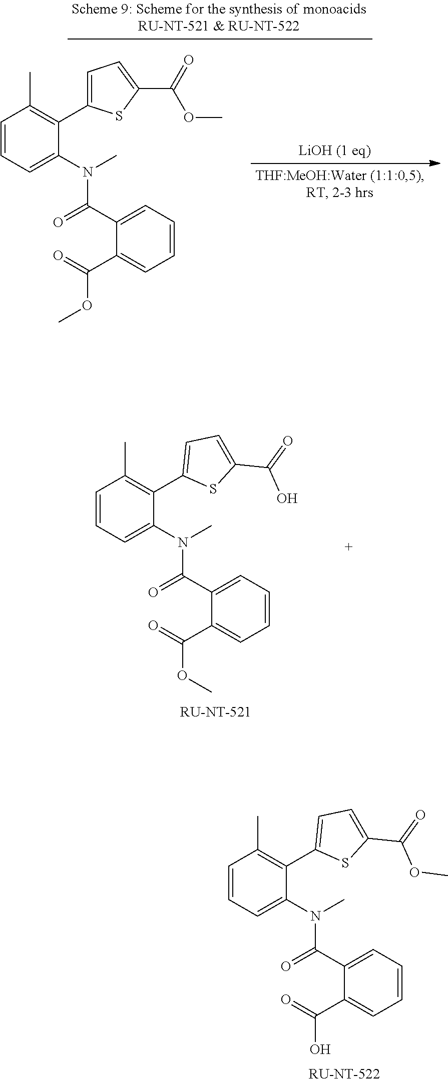 [SMILES: COC(=O)c1ccc(-c2c(C)cccc2N(C)C(=O)c2ccccc2C(=O)OC)s1 ; COC(=O)c1ccccc1C(=O)N(C)c1cccc(C)c1-c1ccc(C(=O)O)s1 ; COC(=O)c1ccc(-c2c(C)cccc2N(C)C(=O)c2ccccc2C(=O)O)s1]

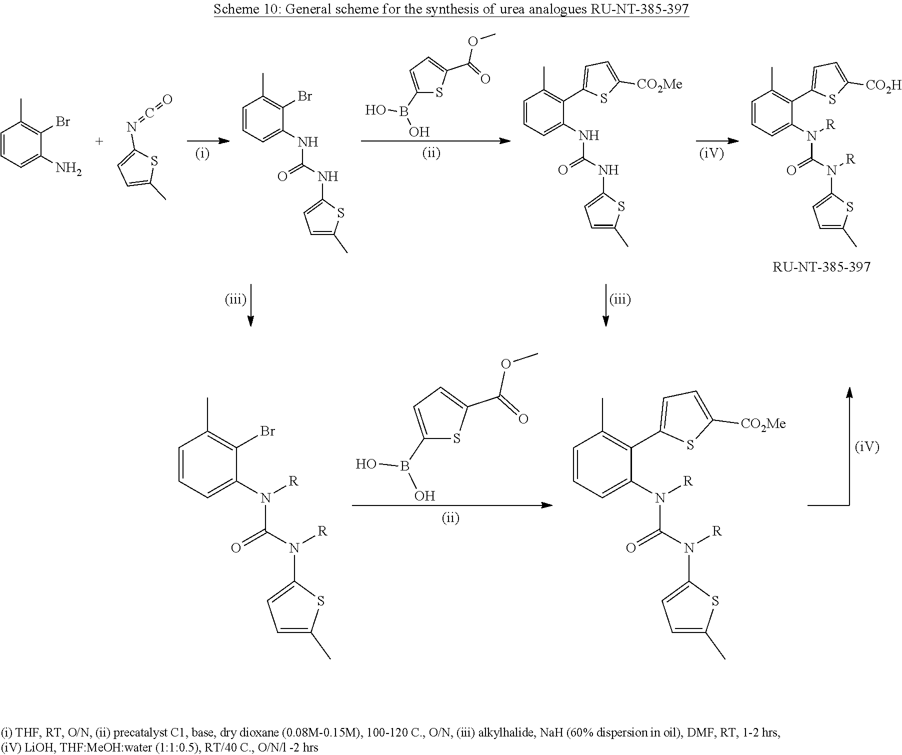

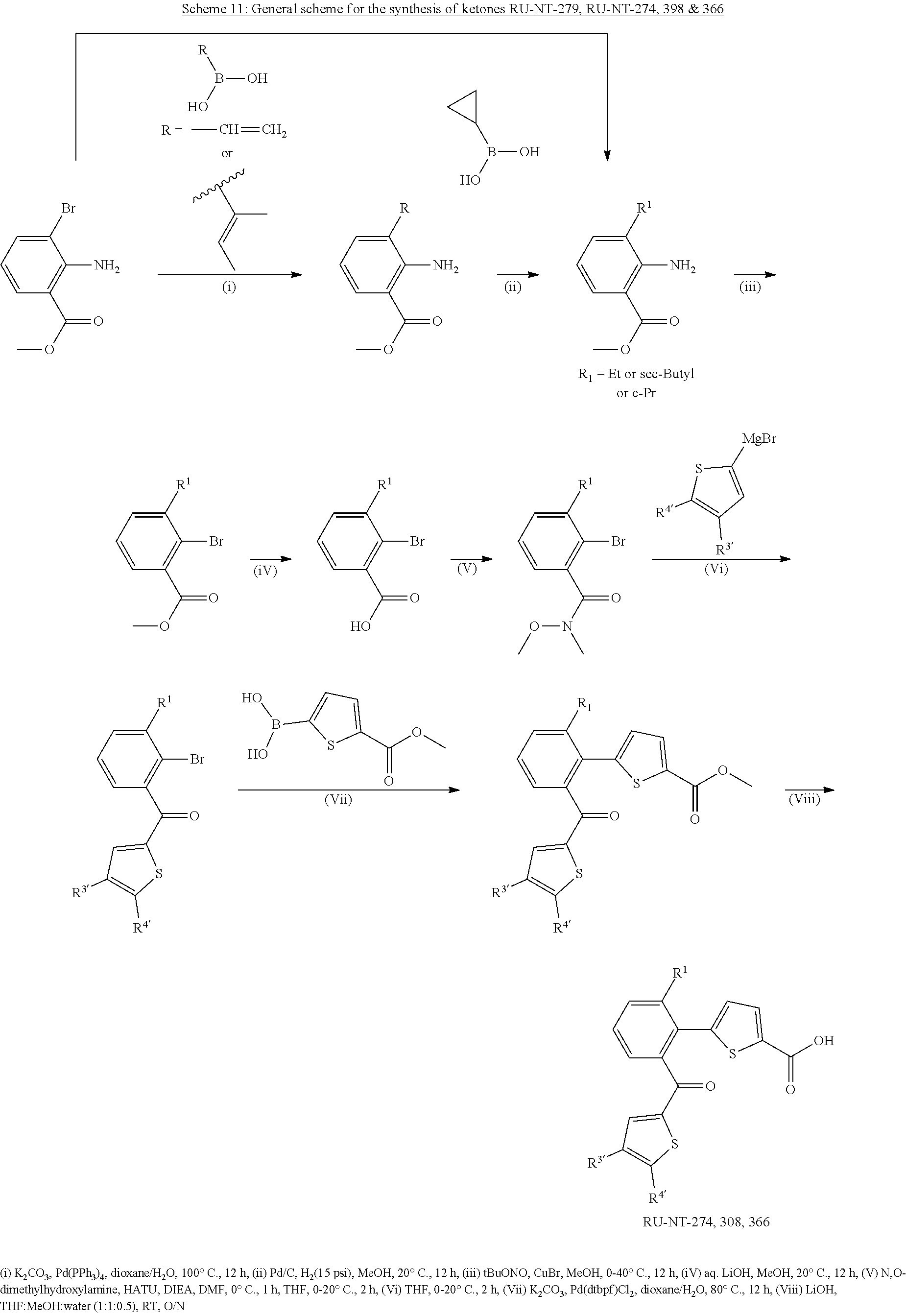

Certain exemplary compounds of the disclosure, including compounds of Formula (I), (II), and/or (III), are provided in Table 1.

TABLE 1
Exemplary compounds of the disclosure

| Compound | Structure | Nomenclature |
|---|---|---|
| CC10501 | 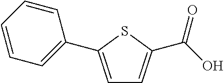 [SMILES: O=C(O)c1ccc(-c2ccccc2)s1] | 5-phenylthiophene-2-carboxylic acid |
| PD00589 | 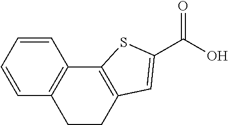 [SMILES: O=C(O)c1cc2c(s1)-c1ccccc1CC2] | 4,5-dihydronaphtho[1,2-b]thiophene-2- carboxylic acid |
| PD00633 | 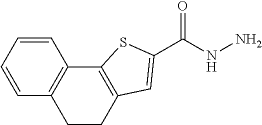 [SMILES: NNC(=O)c1cc2c(s1)-c1ccccc1CC2] | 4,5-dihydronaphtho[1,2-b]thiophene-2- carbohydrazide |
| SEW01689 | 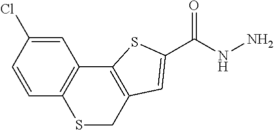 [SMILES: NNC(=O)c1cc2c(s1)-c1cc(Cl)ccc1SC2] | 8-chloro-4H-thieno[3,2-c]thiochromene-2- carbohydrazide |
| SEW01765 | 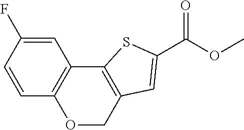 [SMILES: COC(=O)c1cc2c(s1)-c1cc(F)ccc1OC2] | methyl 8-fluoro-4H-thieno[3,2- c]chromene-2-carboxylate |
| SEW02679 | 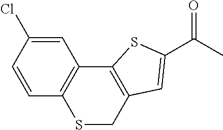 [SMILES: CC(=O)c1cc2c(s1)-c1cc(Cl)ccc1SC2] | 1-(8-chloro-4H-thieno[3,2- c]thiochromen-2-yl)ethan-1-one |
| SEW01776 | 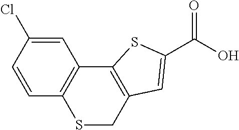 [SMILES: O=C(O)c1cc2c(s1)-c1cc(Cl)ccc1SC2] | 8-chloro-4H-thieno[3,2- c]thiochromene-2-carboxylic acid |
| RU-NT-59 | 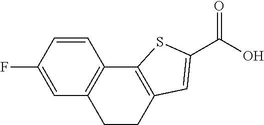 [SMILES: O=C(O)c1cc2c(s1)-c1ccc(F)cc1CC2] | 7-fluoro-4,5-dihydronaphtho[1,2- b]thiophene-2-carboxylic acid |
| RU-NT-61 | 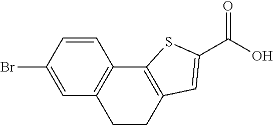 [SMILES: O=C(O)c1cc2c(s1)-c1ccc(Br)cc1CC2] | 7-bromo-4,5-dihydronaphtho[1,2- b]thiophene-2-carboxylic acid |
| RU-NT-62 | 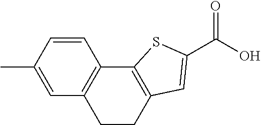 [SMILES: Cc1ccc2c(c1)CCc1cc(C(=O)O)sc1-2] | 7-methyl-4,5-dihydronaphtho[1,2- b]thiophene-2-carboxylic acid |
| RU-NT-63 | 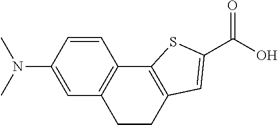 [SMILES: CN(C)c1ccc2c(c1)CCc1cc(C(=O)O)sc1-2] | 7-(dimethylamino)-4,5- dihydronaphtho[1,2- b]thiophene-2-carboxylic acid |
| RU-NT-64 | 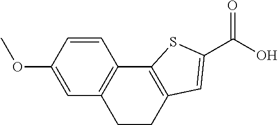 [SMILES: COc1ccc2c(c1)CCc1cc(C(=O)O)sc1-2] | 7-methoxy-4,5-dihydronaphtho [1,2-b]thiophene-2-carboxylic acid |
| RU-NT-70 | 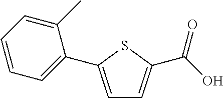 | 5-(o-tolyl)thiophene-2-carboxylic acid |
| RU-NT-75 | 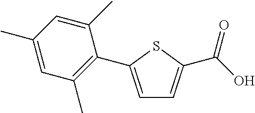 [SMILES: Cc1cc(C)c(-c2ccc(C(=O)O)s2)c(C)c1] | 5-mesitylthiophene-2-carboxylic acid |
| RU-NT-93 | 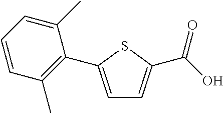 [SMILES: Cc1cccc(C)c1-c1ccc(C(=O)O)s1] | 5-(2,6-dimethylphenyl)thiophene-2- carboxylic acid |
| RU-NT-94 | 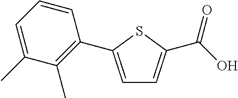 [SMILES: Cc1cccc(-c2ccc(C(=O)O)s2)c1C] | 5-(2,3-dimethylphenyl)thiophene-2- carboxylic acid |
| RU-NT- 116 | 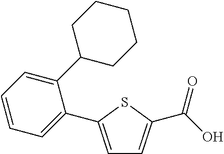 [SMILES: O=C(O)c1ccc(-c2ccccc2C2CCCCC2)s1] | 5-(2-cyclohexylphenyl)thiophene-2- carboxylic acid |
| RU-NT- 120 | 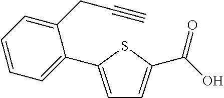 [SMILES: C#CCc1ccccc1-c1ccc(C(=O)O)s1] | 5-(2-(prop-2-yn-1-yl)phenyl)thiophene-2- carboxylic acid |
| RU-NT- 124 | 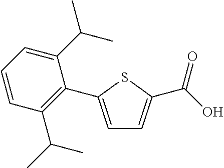 [SMILES: CC(C)c1cccc(C(C)C)c1-c1ccc(C(=O)O)s1] | 5-(2,6-diisopropylphenyl)thiophene-2- carboxylic acid |
| RU-NT- 139 | 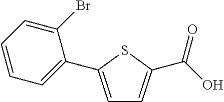 [SMILES: O=C(O)c1ccc(-c2ccccc2Br)s1] | 5-(2-bromophenyl)thiophene-2-carboxylic acid |
| RU-NT- 159 | 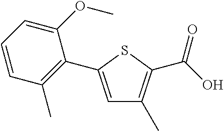 [SMILES: COc1cccc(C)c1-c1cc(C)c(C(=O)O)s1] | 5-(2-methoxy-6-methylphenyl)-3- methylthiophene-2-carboxylic acid |
| RU-NT- 165 | 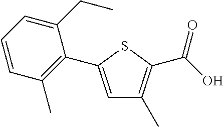 [SMILES: CCc1cccc(C)c1-c1cc(C)c(C(=O)O)s1] | 5-(2-ethyl-6-methylphenyl)thiophene-2- carboxylic acid |
| RU-NT- 183 | 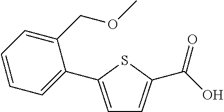 [SMILES: COCc1ccccc1-c1ccc(C(=O)O)s1] | 5-(2-(methoxymethyl)phenyl)thiophene-2- carboxylic acid |
| RU-NT- 186 | 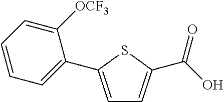 [SMILES: O=C(O)c1ccc(-c2ccccc2OC(F)(F)F)s1] | 5-(2-(trifluoromethoxy)phenyl)thiophene-2- carboxylic acid |
| RU-NT- 192 | 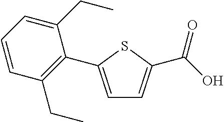 [SMILES: CCc1cccc(CC)c1-c1ccc(C(=O)O)s1] | 5-(2,6-diethylphenyl)thiophene-2- carboxylic acid |
| RU-NT- 198 | 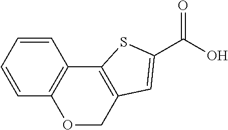 [SMILES: O=C(O)c1cc2c(s1)-c1ccccc1OC2] | 4H-thieno[3,2-c]chromene-2-carboxylic acid |
| RUNT- 199 | 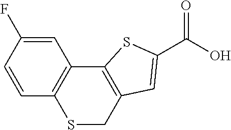 [SMILES: O=C(O)c1cc2c(s1)-c1cc(F)ccc1SC2] | 8-fluoro-4H-thieno[3,2- c]thiochromene-2- carboxylic acid |
| RU-NT- 201 | 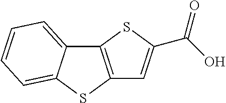 [SMILES: O=C(O)c1cc2sc3ccccc3c2s1] | benzo[b]thieno[2,3-d]thiophene-2- carboxylic acid |
| RU-NT- 202 | 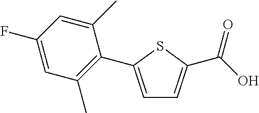 [SMILES: Cc1cc(F)cc(C)c1-c1ccc(C(=O)O)s1] | 5-(4-fluoro-2,6-dimethylphenyl) thiophene-2-arboxylic acid |
| RU-NT- 203 | 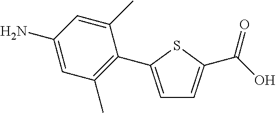 [SMILES: Cc1cc(N)cc(C)c1-c1ccc(C(=O)O)s1] | 5-(4-amino-2,6- dimethylphenyl)thiophene- 2-carboxylic acid |
| RU-NT- 206 | 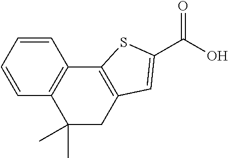 [SMILES: CC1(C)Cc2cc(C(=O)O)sc2-c2ccccc21] | 5,5-dimethyl-4,5-dihydronaphtho[1,2- b]thiophene-2-carboxylic acid |
| RU-NT- 231 | 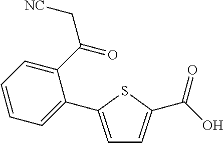 [SMILES: N#CCC(=O)c1ccccc1-c1ccc(C(=O)O)s1] | 5-(2-(2-cyanoacetyl)phenyl)thiophene-2- carboxylic acid |
| RU-NT- 245 | 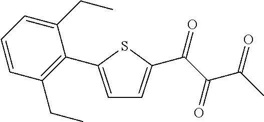 [SMILES: CCc1cccc(CC)c1-c1ccc(C(=O)C(=O)C(C)=O)s1] | 1-(5-(2,6-diethylphenyl)thiophen-2- yl)butane-1,2,3-trione |
| RU-NT- 246 | 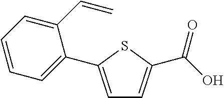 [SMILES: C=Cc1ccccc1-c1ccc(C(=O)O)s1] | 5-(2-vinylphenyl)thiophene-2-carboxylic acid |
| RU-NT- 247 | 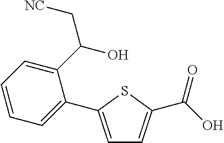 [SMILES: N#CCC(O)c1ccccc1-c1ccc(C(=O)O)s1] | 5-(2-(2-cyano-1-hydroxyethyl)phenyl) thiophene-2-carboxylic acid |
| RU-NT- 248 | 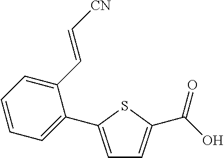 [SMILES: N#C/C=C/c1ccccc1-c1ccc(C(=O)O)s1] | (E)-5-(2-(2-cyanovinyl)phenyl)thiophene- 2-carboxylic acid |
| RU-NT- 249 | 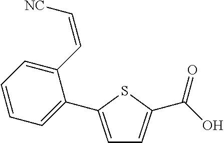 [SMILES: N#C/C=C\c1ccccc1-c1ccc(C(=O)O)s1] | (Z)-5-(2-(2-cyanovinyl)phenyl)thiophene- 2-carboxylic acid |
| RU-NT- 250 | 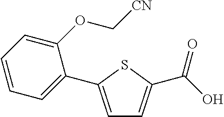 [SMILES: N#CCOc1ccccc1-c1ccc(C(=O)O)s1] | 5-(2-(cyanomethoxy)phenyl)thiophene-2- carboxylic acid |
| RU-NT- 252 | 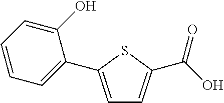 [SMILES: O=C(O)c1ccc(-c2ccccc2O)s1] | 5-(2-hydroxyphenyl)thiophene-2-carboxylic acid |
| RU-NT- 253 | 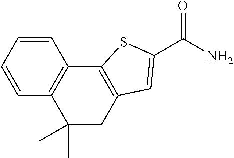 [SMILES: CC1(C)Cc2cc(C(N)=O)sc2-c2ccccc21] | 5,5-dimethyl-4,5-dihydronaphtho[1,2- b]thiophene-2-carboxamide |
| RU-NT- 254 | 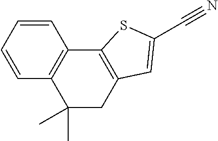 [SMILES: CC1(C)Cc2cc(C#N)sc2-c2ccccc21] | 5,5-dimethyl-4,5-dihydronaphtho[1,2- b]thiophene-2-carbonitrile |
| RU-NT- 255 | 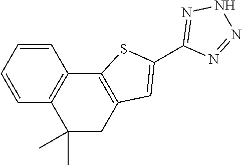 [SMILES: CC1(C)Cc2cc(-c3nn[nH]n3)sc2-c2ccccc21] | 5-(5,5-dimethyl-4,5-dihydronaphtho[1,2- b]thiophen-2-yl)-2H-tetrazole |
| RU-NT- 256 | 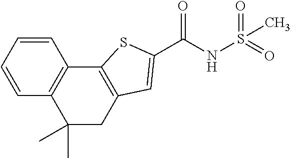 [SMILES: CC1(C)Cc2cc(C(=O)NS(C)(=O)=O)sc2-c2ccccc21] | 5,5-dimethyl-N-(methylsulfonyl)-4,5- dihydronaphtho[1,2-b]thiophene-2- carboxamide |
| RU-NT- 274 | 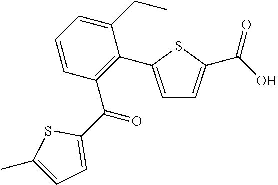 [SMILES: CCc1cccc(C(=O)c2ccc(C)s2)c1-c1ccc(C(=O)O)s1] | 5-(2-ethyl-6-(5-methylthiophene-2- carbonyl)phenyl)thiophene-2-carboxylic acid |
| RU-NT- 281 | 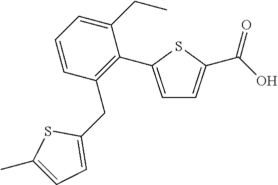 [SMILES: CCc1cccc(Cc2ccc(C)s2)c1-c1ccc(C(=O)O)s1] | 5-(2-ethyl-6-((5-methylthiophen-2- yl)methyl)phenyl)thiophene-2-carboxylic acid |
| RU-NT- 283 | 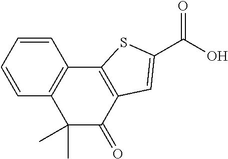 [SMILES: CC1(C)C(=O)c2cc(C(=O)O)sc2-c2ccccc21] | 5,5-dimethyl-4-oxo- 4,5-dihydronaphtho[1,2- b]thiophene-2-carboxylic acid |
| RU-NT- 285 | 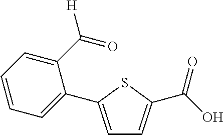 [SMILES: O=Cc1ccccc1-c1ccc(C(=O)O)s1] | 5-(2-formylphenyl)thiophene-2-carboxylic acid |
| RU-NT- 286 | 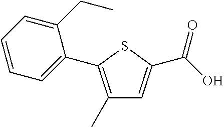 [SMILES: CCc1ccccc1-c1sc(C(=O)O)cc1C] | 5-(2-ethylphenyl)-4-methylthiophene-2- carboxylic acid |
| RU-NT- 287 | 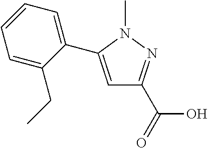 [SMILES: CCc1ccccc1-c1cc(C(=O)O)nn1C] | 5-(2-ethylphenyl)-1-methyl-1H-pyrazole-3- carboxylic acid |
| RU-NT- 288 | 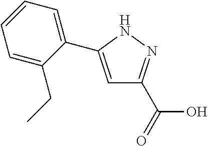 [SMILES: CCc1ccccc1-c1cc(C(=O)O)n[nH]1] | 5-(2-ethylphenyl)-1H-pyrazole-3-carboxylic acid |
| RU-NT- 289 | 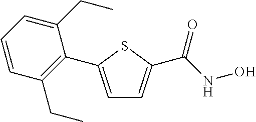 [SMILES: CCc1cccc(CC)c1-c1ccc(C(=O)NO)s1] | 5-(2,6-diethylphenyl)-N-hydroxythiophene- 2-carboxamide |
| RU-NT- 290 | 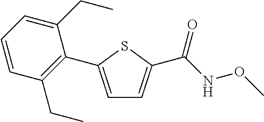 [SMILES: CCc1cccc(CC)c1-c1ccc(C(=O)NOC)s1] | 5-(2,6-diethylphenyl)-N-methoxythiophene- 2-carboxamide |
| RU-NT- 291 | 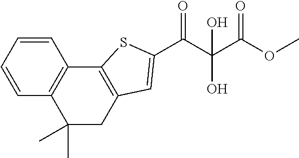 [SMILES: COC(=O)C(O)(O)C(=O)c1cc2c(s1)-c1ccccc1C(C)(C)C2] | methyl 3-(5,5-dimethyl-4,5- dihydronaphtho[1,2-b]thiophen-2-yl)-2,2- dihydroxy-3-oxopropanoate |
| RU-NT- 293 | 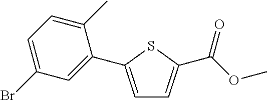 [SMILES: COC(=O)c1ccc(-c2cc(Br)ccc2C)s1] | methyl 5-(5-bromo-2- methylphenyl)thiophene-2-carboxylate |
| RU-NT- 294 | 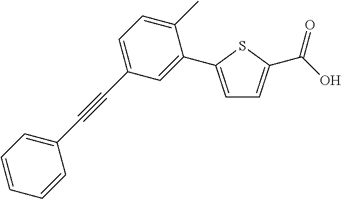 [SMILES: Cc1ccc(C#Cc2ccccc2)cc1-c1ccc(C(=O)O)s1] | 5-(2-methyl-5- (phenylethynyl)phenyl)thiophene-2- carboxylic acid |
| RU-NT- 295 | 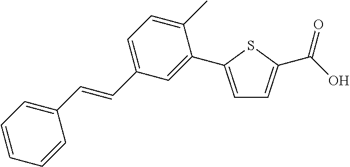 [SMILES: Cc1ccc(/C=C/c2ccccc2)cc1-c1ccc(C(=O)O)s1] | (E)-5-(2-methyl-5-styrylphenyl)thiophene-2- carboxylic acid |
| RU-NT- 296 | 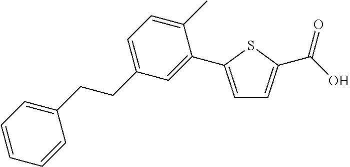 [SMILES: Cc1ccc(CCc2ccccc2)cc1-c1ccc(C(=O)O)s1] | 5-(2-methyl-5-phenethylphenyl)thiophene-2- carboxylic acid |
| RU-NT- 297 | 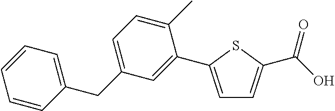 [SMILES: Cc1ccc(Cc2ccccc2)cc1-c1ccc(C(=O)O)s1] | 5-(5-benzyl-2-methylphenyl)thiophene-2- carboxylic acid |
| RU-NT- 298 | 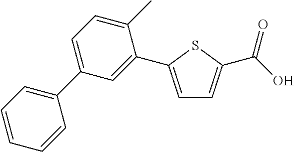 [SMILES: Cc1ccc(-c2ccccc2)cc1-c1ccc(C(=O)O)s1] | 5-(4-methyl-[1,1′-biphenyl]-3-yl)thiophene- 2-carboxylic acid |
| RU-NT- 299 | 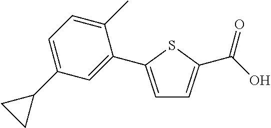 [SMILES: Cc1ccc(C2CC2)cc1-c1ccc(C(=O)O)s1] | 5-(5-cyclopropyl-2-methylphenyl)thiophene- 2-carboxylic acid |
| RU-NT- 300 | 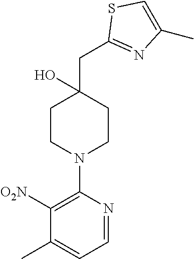 | 1-(4-methyl-3-nitropyridin-2-yl)-4-((4- methylthiazol-2-yl)methyl)piperidin-4-ol |
| RU-NT- 301 | 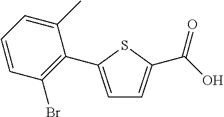 [SMILES: Cc1cccc(Br)c1-c1ccc(C(=O)O)s1] | 5-(2-bromo-6-methylphenyl)thiophene-2- carboxylic acid |
| RU-NT- 302 | 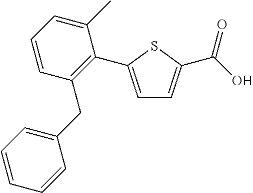 [SMILES: Cc1cccc(Cc2ccccc2)c1-c1ccc(C(=O)O)s1] | 5-(2-benzyl-6-methylphenyl)thiophene-2- carboxylic acid |
| RU-NT- 303 | 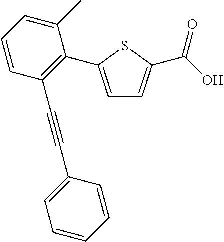 [SMILES: Cc1cccc(C#Cc2ccccc2)c1-c1ccc(C(=O)O)s1] | 5-(2-methyl-6- (phenylethynyl)phenyl)thiophene-2- carboxylic acid2 |
| RU-NT- 304 | 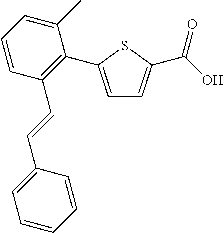 [SMILES: Cc1cccc(/C=C/c2ccccc2)c1-c1ccc(C(=O)O)s1] | (E)-5-(2-methyl-6-styrylphenyl)thiophene-2- carboxylic acid |
| RU-NT- 305 | 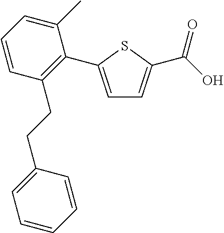 [SMILES: Cc1cccc(CCc2ccccc2)c1-c1ccc(C(=O)O)s1] | 5-(2-methyl-6-phenethylphenyl)thiophene-2- carboxylic acid |
| RU-NT- 306 | 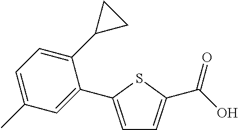 [SMILES: Cc1ccc(C2CC2)c(-c2ccc(C(=O)O)s2)c1] | 5-(2-cyclopropyl-5-methylphenyl)thiophene- 2-carboxylic acid |
| RU-NT- 307 | 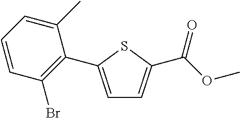 [SMILES: COC(=O)c1ccc(-c2c(C)cccc2Br)s1] | methyl 5-(2-bromo-6- methylphenyl)thiophene-2-carboxylate |
| RU-NT- 343 | 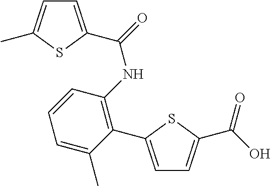 [SMILES: Cc1ccc(C(=O)Nc2cccc(C)c2-c2ccc(C(=O)O)s2)s1] | 5-(2-methyl-6-(5-methylthiophene-2- carboxamido)phenyl)thiophene-2-carboxylic acid |
| RU-NT- 344 | 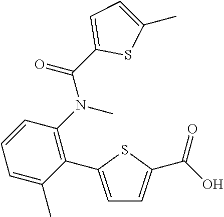 [SMILES: Cc1ccc(C(=O)N(C)c2cccc(C)c2-c2ccc(C(=O)O)s2)s1] | 5-(2-(N,5-dimethylthiophene-2- carboxamido)-6-methylphenyl)thiophene-2- carboxylic acid |
| RU-NT- 345 | 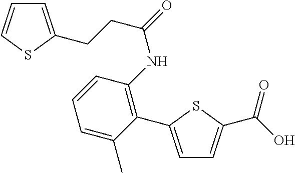 [SMILES: Cc1cccc(NC(=O)CCc2cccs2)c1-c1ccc(C(=O)O)s1] | 5-(2-methyl-6-(3-(thiophen-2- yl)propanamido)phenyl)thiophene-2- carboxylic acid |
| RU-NT- 358 | 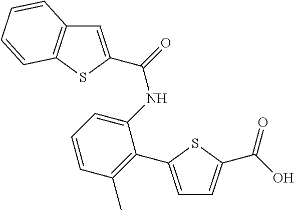 [SMILES: Cc1cccc(NC(=O)c2cc3ccccc3s2)c1-c1ccc(C(=O)O)s1] | 5-(2-(benzo[b]thiophene-2-carboxamido)-6- methylphenyl)thiophene-2-carboxylic acid |
| RU-NT- 359 | 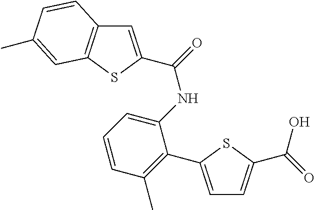 [SMILES: Cc1ccc2cc(C(=O)Nc3cccc(C)c3-c3ccc(C(=O)O)s3)sc2c1] | 5-(2-methyl-6-(6-methylbenzo[b]thiophene- 2-carboxamido)phenyl)thiophene-2- carboxylic acid |
| RU-NT- 360 | 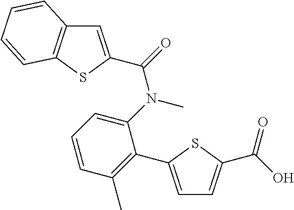 [SMILES: Cc1cccc(N(C)C(=O)c2cc3ccccc3s2)c1-c1ccc(C(=O)O)s1] | 5-(2-methyl-6-(N-methylbenzo[b]thiophene- 2-carboxamido)phenyl)thiophene-2- carboxylic acid |
| RU-NT- 361 | 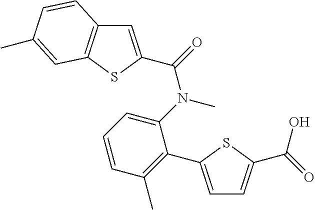 [SMILES: Cc1ccc2cc(C(=O)N(C)c3cccc(C)c3-c3ccc(C(=O)O)s3)sc2c1] | 5-(2-(N,6-dimethylbenzo[b]thiophene-2- carboxamido)-6-methylphenyl)thiophene-2- carboxylic acid |
| RU-NT- 367 | 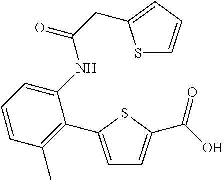 [SMILES: Cc1cccc(NC(=O)Cc2cccs2)c1-c1ccc(C(=O)O)s1] | 5-(2-methyl-6-(2-(thiophen-2- yl)acetamido)phenyl)thiophene-2-carboxylic acid |
| RU-NT- 368 | 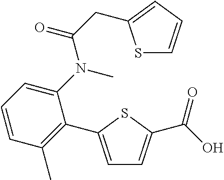 [SMILES: Cc1cccc(N(C)C(=O)Cc2cccs2)c1-c1ccc(C(=O)O)s1] | 5-(2-methyl-6-(N-methyl-2-(thiophen-2- yl)acetamido)phenyl)thiophene-2-carboxylic acid |
| RU-NT- 369 | 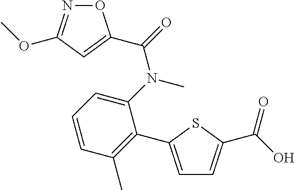 [SMILES: COc1cc(C(=O)N(C)c2cccc(C)c2-c2ccc(C(=O)O)s2)on1] | 5-(2-(3-methoxy-N-methylisoxazole-5- carboxamido)-6-methylphenyl)thiophene-2- carboxylic acid |
| RU-NT- 370 | 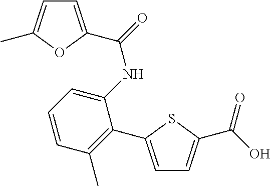 [SMILES: Cc1ccc(C(=O)Nc2cccc(C)c2-c2ccc(C(=O)O)s2)o1] | 5-(2-methyl-6-(5-methylfuran-2- carboxamido)phenyl)thiophene-2-carboxylic acid |
| RU-NT- 371 | 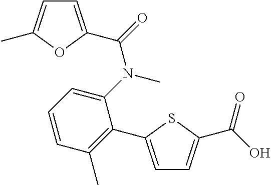 [SMILES: Cc1ccc(C(=O)N(C)c2cccc(C)c2-c2ccc(C(=O)O)s2)o1] | 5-(2-(N,5-dimethylfuran-2-carboxamido)-6- methylphenyl)thiophene-2-carboxylic acid |
| RU-NT- 372 | 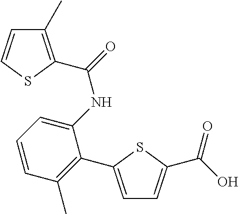 [SMILES: Cc1ccsc1C(=O)Nc1cccc(C)c1-c1ccc(C(=O)O)s1] | 5-(2-methyl-6-(3-methylthiophene-2- carboxamido)phenyl)thiophene-2-carboxylic acid |
| RU-NT- 373 | 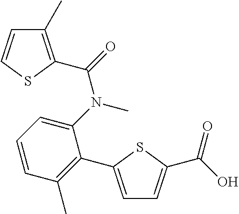 [SMILES: Cc1ccsc1C(=O)N(C)c1cccc(C)c1-c1ccc(C(=O)O)s1] | 5-(2-(N,3-dimethylthiophene-2- carboxamido)-6-methylphenyl)thiophene-2- carboxylic acid |
| RU-NT- 374 | 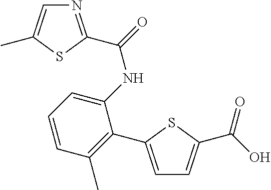 [SMILES: Cc1cnc(C(=O)Nc2cccc(C)c2-c2ccc(C(=O)O)s2)s1] | 5-(2-methyl-6-(5-methylthiazole-2- carboxamido)phenyl)thiophene-2-carboxylic acid |
| RU-NT- 375 | 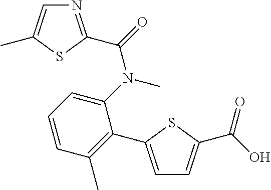 [SMILES: Cc1cnc(C(=O)N(C)c2cccc(C)c2-c2ccc(C(=O)O)s2)s1] | 5-(2-(N,5-dimethylthiazole-2-carboxamido)- 6-methylphenyl)thiophene-2-carboxylic acid |
| RU-NT- 376 | 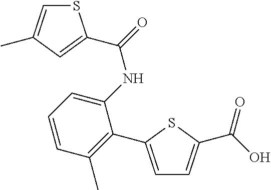 [SMILES: Cc1csc(C(=O)Nc2cccc(C)c2-c2ccc(C(=O)O)s2)c1] | 5-(2-methyl-6-(4-methylthiophene-2- carboxamido)phenyl)thiophene-2-carboxylic acid |
| RU-NT- 377 | 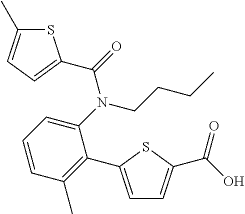 [SMILES: CCCCN(C(=O)c1ccc(C)s1)c1cccc(C)c1-c1ccc(C(=O)O)s1] | 5-(2-(N-butyl-5-methylthiophene-2- carboxamido)-6-methylphenyl)thiophene-2- carboxylic acid |
| RU-NT- 379 | 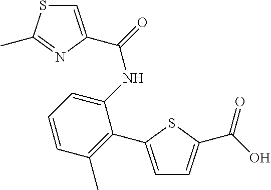 [SMILES: Cc1nc(C(=O)Nc2cccc(C)c2-c2ccc(C(=O)O)s2)cs1] | 5-(2-methyl-6-(2-methylthiazole-4- carboxamido)phenyl)thiophene-2-carboxylic acid |
| RU-NT- 380 | 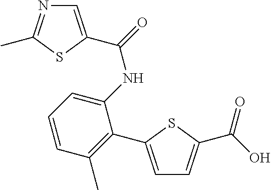 [SMILES: Cc1ncc(C(=O)Nc2cccc(C)c2-c2ccc(C(=O)O)s2)s1] | 5-(2-methyl-6-(2-methylthiazole-5- carboxamido)phenyl)thiophene-2-carboxylic acid |
| RU-NT- 381 | 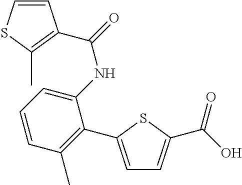 [SMILES: Cc1cccc(NC(=O)c2ccsc2C)c1-c1ccc(C(=O)O)s1] | 5-(2-methyl-6-(2-methylthiophene-3- carboxamido)phenyl)thiophene-2-carboxylic acid |
| RU-NT- 382 | 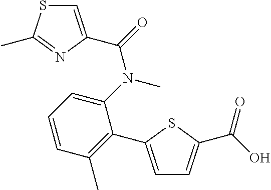 [SMILES: Cc1nc(C(=O)N(C)c2cccc(C)c2-c2ccc(C(=O)O)s2)cs1] | 5-(2-(N,2-dimethylthiazole-4-carboxamido)- 6-methylphenyl)thiophene-2-carboxylic acid |
| RU-NT- 383 | 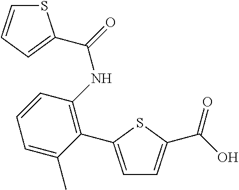 [SMILES: Cc1cccc(NC(=O)c2cccs2)c1-c1ccc(C(=O)O)s1] | 5-(2-methyl-6-(thiophene-2- carboxamido)phenyl)thiophene-2-carboxylic acid |
| RU-NT- 384 | 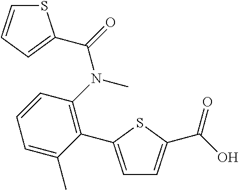 [SMILES: Cc1cccc(N(C)C(=O)c2cccs2)c1-c1ccc(C(=O)O)s1] | 5-(2-methyl-6-(N-methylthiophene-2- carboxamido)phenyl)thiophene-2-carboxylic acid |
| RU-NT- 385 | 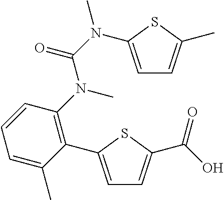 [SMILES: Cc1ccc(N(C)C(=O)N(C)c2cccc(C)c2-c2ccc(C(=O)O)s2)s1] | 5-(2-(1,3-dimethyl-3-(5-methylthiophen-2- yl)ureido)-6-methylphenyl)thiophene-2- carboxylic acid |
| RU-NT- 387 | 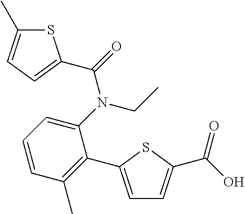 [SMILES: CCN(C(=O)c1ccc(C)s1)c1cccc(C)c1-c1ccc(C(=O)O)s1] | 5-(2-(N-ethyl-5-methylthiophene-2- carboxamido)-6-methylphenyl)thiophene-2- carboxylic acid |
| RU-NT- 388 | 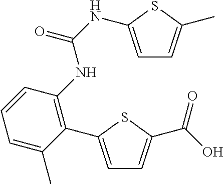 [SMILES: Cc1ccc(NC(=O)Nc2cccc(C)c2-c2ccc(C(=O)O)s2)s1] | 5-(2-methyl-6-(3-(5-methylthiophen-2- yl)ureido)phenyl)thiophene-2-carboxylic acid |
| RU-NT- 392 | 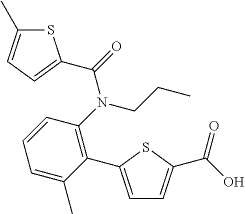 [SMILES: CCCN(C(=O)c1ccc(C)s1)c1cccc(C)c1-c1ccc(C(=O)O)s1] | 5-(2-methyl-6-(5-methyl-N- propylthiophene-2- carboxamido)phenyl)thiophene-2-carboxylic acid |
| RU-NT- 393 | 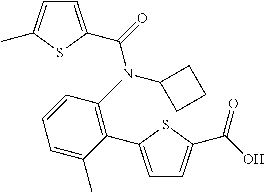 [SMILES: Cc1ccc(C(=O)N(c2cccc(C)c2-c2ccc(C(=O)O)s2)C2CCC2)s1] | 5-(2-(N-cyclobutyl-5-methylthiophene-2- carboxamido)-6-methylphenyl)thiophene-2- carboxylic acid |
| RU-NT- 394 | 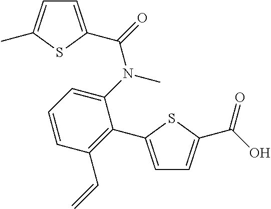 [SMILES: C=Cc1cccc(N(C)C(=O)c2ccc(C)s2)c1-c1ccc(C(=O)O)s1] | 5-(2-(N,5-dimethylthiophene-2- carboxamido)-6-vinylphenyl)thiophene-2- carboxylic acid |
| RU-NT- 395 | 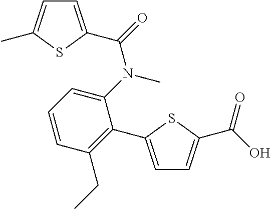 [SMILES: CCc1cccc(N(C)C(=O)c2ccc(C)s2)c1-c1ccc(C(=O)O)s1] | 5-(2-(N,5-dimethylthiophene-2- carboxamido)-6-ethylphenyl)thiophene-2- carboxylic acid |
| RU-NT- 396 | 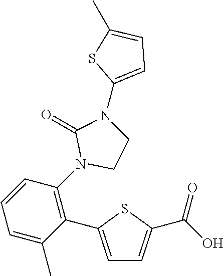 [SMILES: Cc1ccc(N2CCN(c3cccc(C)c3-c3ccc(C(=O)O)s3)C2=O)s1] | 5-(2-methyl-6-(3-(5-methylthiophen-2-yl)-2- oxoimidazolidin-1-yl)phenyl)thiophene-2- carboxylic acid |
| RU-NT- 397 | 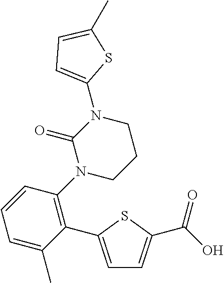 [SMILES: Cc1ccc(N2CCCN(c3cccc(C)c3-c3ccc(C(=O)O)s3)C2=O)s1] | 5-(2-methyl-6-(3-(5-methylthiophen-2-yl)-2- oxotetrahydropyrimidin-1(2H)- yl)phenyl)thiophene-2-carboxylic acid |
| RU-NT- 398 | 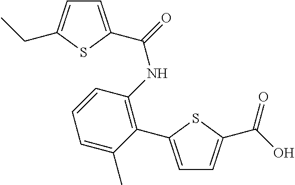 [SMILES: CCc1ccc(C(=O)Nc2cccc(C)c2-c2ccc(C(=O)O)s2)s1] | 5-(2-(5-ethylthiophene-2-carboxamido)-6- methylphenyl)thiophene-2-carboxylic acid |
| RU-NT- 399 | 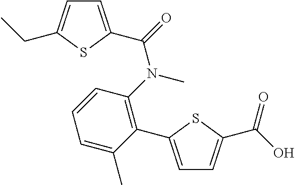 [SMILES: CCc1ccc(C(=O)N(C)c2cccc(C)c2-c2ccc(C(=O)O)s2)s1] | 5-(2-(5-ethyl-N-methylthiophene-2- carboxamido)-6-methylphenyl)thiophene-2- carboxylic acid |
| RU-NT- 400 | 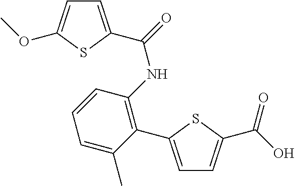 [SMILES: COc1ccc(C(=O)Nc2cccc(C)c2-c2ccc(C(=O)O)s2)s1] | 5-(2-(5-methoxythiophene-2-carboxamido)- 6-methylphenyl)thiophene-2-carboxylic acid |
| RU-NT- 401 | 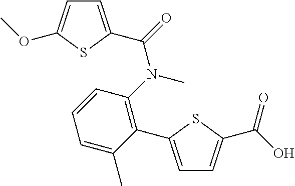 [SMILES: COc1ccc(C(=O)N(C)c2cccc(C)c2-c2ccc(C(=O)O)s2)s1] | 5-(2-(5-methoxy-N-methylthiophene-2- carboxamido)-6-methylphenyl)thiophene-2- carboxylic acid |
| RU-NT- 402 | 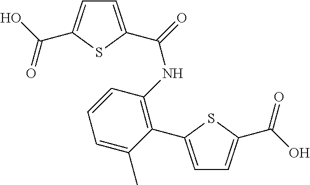 [SMILES: Cc1cccc(NC(=O)c2ccc(C(=O)O)s2)c1-c1ccc(C(=O)O)s1] | 5-((2-(5-carboxythiophen-2-yl)-3- methylphenyl)carbamoyl)thiophene-2- carboxylic acid |
| RU-NT- 403 | 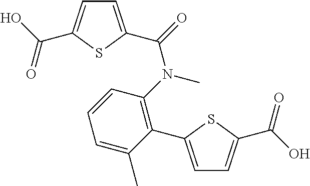 [SMILES: Cc1cccc(N(C)C(=O)c2ccc(C(=O)O)s2)c1-c1ccc(C(=O)O)s1] | 5-(2-(5-carboxy-N-methylthiophene-2- carboxamido)-6-methylphenyl)thiophene-2- carboxylic acid |
| RU-NT- 404 | 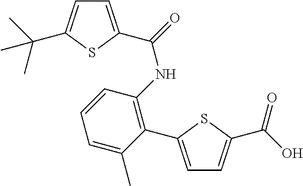 [SMILES: Cc1cccc(NC(=O)c2ccc(C(C)(C)C)s2)c1-c1ccc(C(=O)O)s1] | 5-(2-(5-(tert-butyl)thiophene-2- carboxamido)-6-methylphenyl)thiophene-2- carboxylic acid |
| RU-NT- 406 | 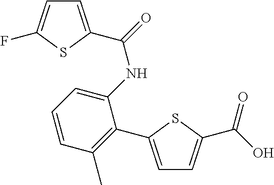 [SMILES: Cc1cccc(NC(=O)c2ccc(F)s2)c1-c1ccc(C(=O)O)s1] | 5-(2-(5-fluorothiophene-2-carboxamido)-6- methylphenyl)thiophene-2-carboxylic acid |
| RU-NT- 407 | 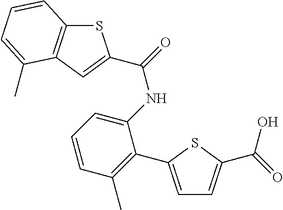 [SMILES: Cc1cccc(NC(=O)c2cc3c(C)cccc3s2)c1-c1ccc(C(=O)O)s1] | 5-(2-methyl-6-(4-methylbenzo[b]thiophene- 2-carboxamido)phenyl)thiophene-2- carboxylic acid |
| RU-NT- 408 | 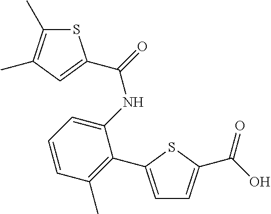 [SMILES: Cc1cc(C(=O)Nc2cccc(C)c2-c2ccc(C(=O)O)s2)sc1C] | 5-(2-(4,5-dimethylthiophene-2- carboxamido)-6-methylphenyl)thiophene-2- carboxylic acid |
| RU-NT- 411 | 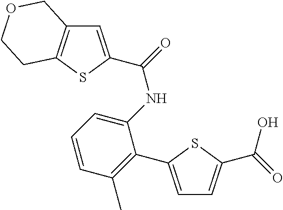 [SMILES: Cc1cccc(NC(=O)c2cc3c(s2)CCOC3)c1-c1ccc(C(=O)O)s1] | 5-(2-(6,7-dihydro-4H-thieno[3,2-c]pyran-2- carboxamido)-6-methylphenyl)thiophene-2- carboxylic acid |
| RU-NT- 412 | 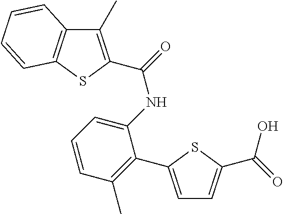 [SMILES: Cc1cccc(NC(=O)c2sc3ccccc3c2C)c1-c1ccc(C(=O)O)s1] | 5-(2-methyl-6-(3-methylbenzo[b]thiophene- 2-carboxamido)phenyl)thiophene-2- carboxylic acid |
| RU-NT- 413 | 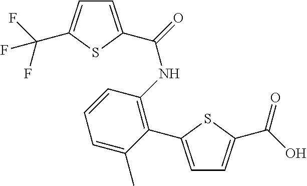 [SMILES: Cc1cccc(NC(=O)c2ccc(C(F)(F)F)s2)c1-c1ccc(C(=O)O)s1] | 5-(2-methyl-6-(5- (trifluoromethyl)thiophene-2- carboxamido)phenyl)thiophene-2-carboxylic acid |
| RU-NT- 414 | 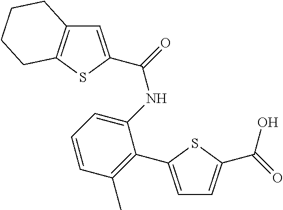 [SMILES: Cc1cccc(NC(=O)c2cc3c(s2)CCCC3)c1-c1ccc(C(=O)O)s1] | 5-(2-methyl-6-(4,5,6,7- tetrahydrobenzo[b]thiophene-2- carboxamido)phenyl)thiophene-2-carboxylic acid |
| RU-NT- 415 | 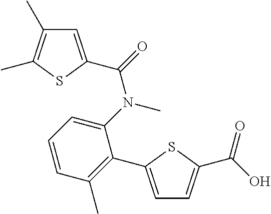 [SMILES: Cc1cc(C(=O)N(C)c2cccc(C)c2-c2ccc(C(=O)O)s2)sc1C] | 5-(2-methyl-6-(N,4,5-trimethylthiophene-2- carboxamido)phenyl)thiophene-2-carboxylic acid |
| RU-NT- 416 | 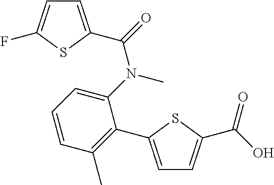 [SMILES: Cc1cccc(N(C)C(=O)c2ccc(F)s2)c1-c1ccc(C(=O)O)s1] | 5-(2-(5-fluoro-N-methylthiophene-2- carboxamido)-6-methylphenyl)thiophene-2- carboxylic acid |
| RU-NT- 417 | 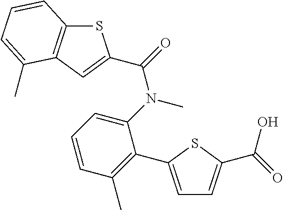 [SMILES: Cc1cccc(N(C)C(=O)c2cc3c(C)cccc3s2)c1-c1ccc(C(=O)O)s1] | 5-(2-(N,4-dimethylbenzo[b]thiophene-2- carboxamido)-6-methylphenyl)thiophene-2- carboxylic acid |
| RU-NT- 418 | 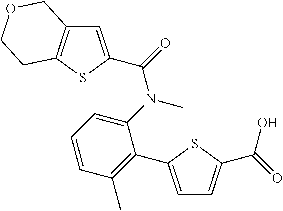 [SMILES: Cc1cccc(N(C)C(=O)c2cc3c(s2)CCOC3)c1-c1ccc(C(=O)O)s1] | 5-(2-methyl-6-(N-methyl-6,7-dihydro-4H- thieno[3,2-c]pyran-2- carboxamido)phenyl)thiophene-2-carboxylic acid |
| RU-NT- 419 | 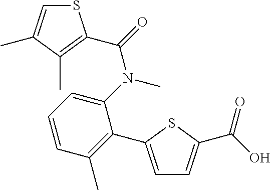 [SMILES: Cc1csc(C(=O)N(C)c2cccc(C)c2-c2ccc(C(=O)O)s2)c1C] | 5-(2-methyl-6-(N,3,4-trimethylthiophene-2- carboxamido)phenyl)thiophene-2-carboxylic acid |
| RU-NT- 420 | 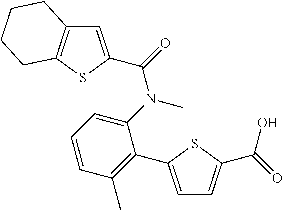 [SMILES: Cc1cccc(N(C)C(=O)c2cc3c(s2)CCCC3)c1-c1ccc(C(=O)O)s1] | 5-(2-methyl-6-(N-methyl-4,5,6,7- tetrahydrobenzo[b]thiophene-2- carboxamido)phenyl)thiophene-2-carboxylic acid |
| RU-NT- 421 | 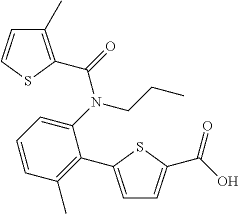 [SMILES: CCCN(C(=O)c1sccc1C)c1cccc(C)c1-c1ccc(C(=O)O)s1] | 5-(2-methyl-6-(3-methyl-N- propylthiophene-2- carboxamido)phenyl)thiophene-2-carboxylic acid |
| RU-NT- 422 | 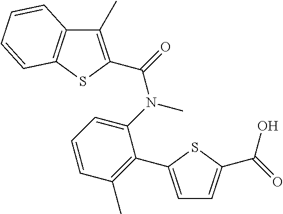 [SMILES: Cc1cccc(N(C)C(=O)c2sc3ccccc3c2C)c1-c1ccc(C(=O)O)s1] | 5-(2-(N,3-dimethylbenzo[b]thiophene-2- carboxamido)-6-methylphenyl)thiophene-2- carboxylic acid |
| RU-NT- 423 | 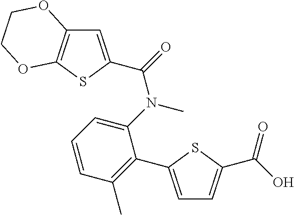 [SMILES: Cc1cccc(N(C)C(=O)c2cc3c(s2)OCCO3)c1-c1ccc(C(=O)O)s1] | 5-(2-methyl-6-(N-methyl-2,3- dihydrothieno[2,3-b][1,4]dioxine-6- carboxamido)phenyl)thiophene-2-carboxylic acid |
| RU-NT- 424 | 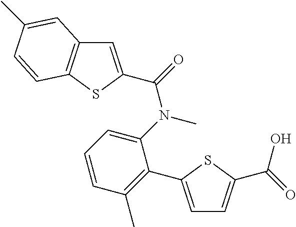 [SMILES: Cc1ccc2sc(C(=O)N(C)c3cccc(C)c3-c3ccc(C(=O)O)s3)cc2c1] | 5-(2-(N,5-dimethylbenzo[b]thiophene-2- carboxamido)-6-methylphenyl)thiophene-2- carboxylic acid |
| RU-NT- 425 | 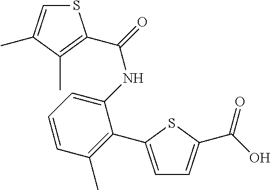 [SMILES: Cc1csc(C(=O)Nc2cccc(C)c2-c2ccc(C(=O)O)s2)c1C] | 5-(2-(3,4-dimethylthiophene-2- carboxamido)-6-methylphenyl)thiophene-2- carboxylic acid |
| RU-NT- 426 | 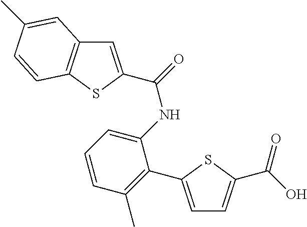 [SMILES: Cc1ccc2sc(C(=O)Nc3cccc(C)c3-c3ccc(C(=O)O)s3)cc2c1] | 5-(2-methyl-6-(5-methylbenzo[b]thiophene- 2-carboxamido)phenyl)thiophene-2- carboxylic acid |
| RU-NT- 428 | 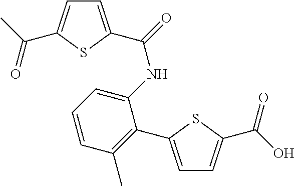 [SMILES: CC(=O)c1ccc(C(=O)Nc2cccc(C)c2-c2ccc(C(=O)O)s2)s1] | 5-(2-(5-acetylthiophene-2-carboxamido)-6- methylphenyl)thiophene-2-carboxylic acid |
| RU-NT- 429 | 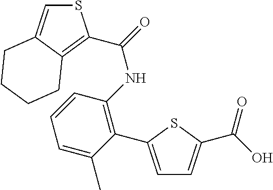 [SMILES: Cc1cccc(NC(=O)c2scc3c2CCCC3)c1-c1ccc(C(=O)O)s1] | 5-(2-methyl-6-(4,5,6,7- tetrahydrobenzo[c]thiophene-1- carboxamido)phenyl)thiophene-2-carboxylic acid |
| RU-NT- 430 | 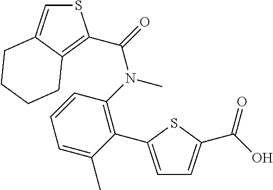 [SMILES: Cc1cccc(N(C)C(=O)c2scc3c2CCCC3)c1-c1ccc(C(=O)O)s1] | 5-(2-methyl-6-(N-methyl-4,5,6,7- tetrahydrobenzo[c]thiophene-1- carboxamido)phenyl)thiophene-2-carboxylic acid |
| RU-NT- 431 | 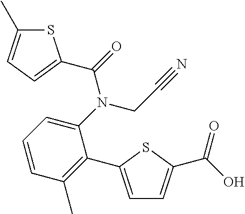 [SMILES: Cc1ccc(C(=O)N(CC#N)c2cccc(C)c2-c2ccc(C(=O)O)s2)s1] | 5-(2-(N-(cyanomethyl)-5-methylthiophene- 2-carboxamido)-6-methylphenyl)thiophene- 2-carboxylic acid |
| RU-NT- 433 | 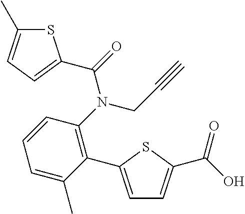 [SMILES: C#CCN(C(=O)c1ccc(C)s1)c1cccc(C)c1-c1ccc(C(=O)O)s1] | 5-(2-methyl-6-(5-methyl-N-(prop-2-yn-1- yl)thiophene-2- carboxamido)phenyl)thiophene-2-carboxylic acid |
| RU-NT- 434 | 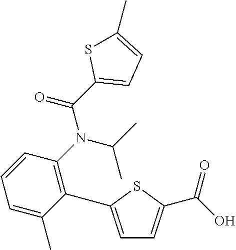 [SMILES: Cc1ccc(C(=O)N(c2cccc(C)c2-c2ccc(C(=O)O)s2)C(C)C)s1] | 5-(2-(N-isopropyl-5-methylthiophene-2- carboxamido)-6-methylphenyl)thiophene-2- carboxylic acid |
| RU-NT- 435 | 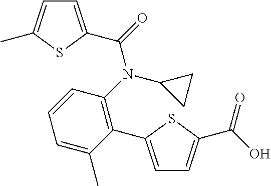 [SMILES: Cc1ccc(C(=O)N(c2cccc(C)c2-c2ccc(C(=O)O)s2)C2CC2)s1] | 5-(2-(N-cyclopropyl-5-methylthiophene-2- carboxamido)-6-methylphenyl)thiophene-2- carboxylic acid |
| RU-NT- 436 | 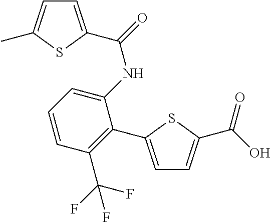 [SMILES: Cc1ccc(C(=O)Nc2cccc(C(F)(F)F)c2-c2ccc(C(=O)O)s2)s1] | 5-(2-(5-methylthiophene-2-carboxamido)-6- (trifluoromethyl)phenyl)thiophene-2- carboxylic acid |
| RU-NT- 437 | 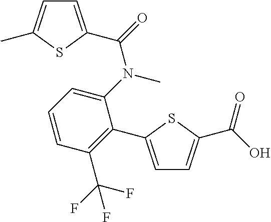 [SMILES: Cc1ccc(C(=O)N(C)c2cccc(C(F)(F)F)c2-c2ccc(C(=O)O)s2)s1] | 5-(2-(N,5-dimethylthiophene-2- carboxamido)-6- (trifluoromethyl)phenyl)thiophene-2- carboxylic acid |
| RU-NT- 438 | 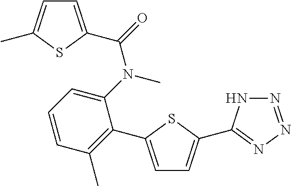 [SMILES: Cc1ccc(C(=O)N(C)c2cccc(C)c2-c2ccc(-c3nnn[nH]3)s2)s1] | N-(2-(5-(1H-tetrazol-5-yl)thiophen-2-yl)-3- methylphenyl)-N,5-dimethylthiophene-2- carboxamide |
| RU-NT- 439 | 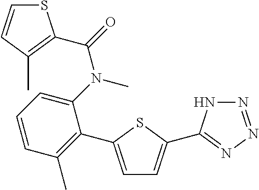 [SMILES: Cc1ccsc1C(=O)N(C)c1cccc(C)c1-c1ccc(-c2nnn[nH]2)s1] | N-(2-(5-(1H-tetrazol-5-yl)thiophen-2-yl)-3- methylphenyl)-N,3-dimethylthiophene-2- carboxamide |
| RU-NT- 440 | 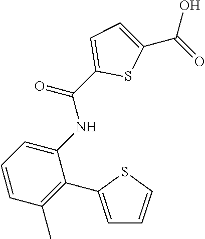 [SMILES: Cc1cccc(NC(=O)c2ccc(C(=O)O)s2)c1-c1cccs1] | 5-((3-methyl-2-(thiophen-2- yl)phenyl)carbamoyl)thiophene-2-carboxylic acid |
| RU-NT- 441 | 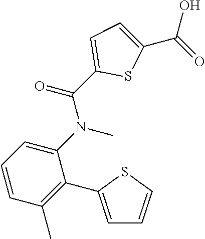 [SMILES: Cc1cccc(N(C)C(=O)c2ccc(C(=O)O)s2)c1-c1cccs1] | 5-(methyl(3-methyl-2-(thiophen-2- yl)phenyl)carbamoyl)thiophene-2-carboxylic acid |
| RU-NT- 442 | 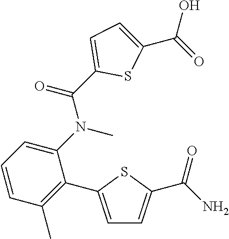 [SMILES: Cc1cccc(N(C)C(=O)c2ccc(C(=O)O)s2)c1-c1ccc(C(N)=O)s1] | 5-((2-(5-carbamoylthiophen-2-yl)-3- methylphenyl)(methyl)carbamoyl)thiophene- 2-carboxylic acid |
| RU-NT- 443 | 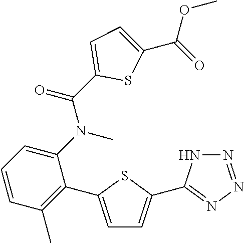 [SMILES: COC(=O)c1ccc(C(=O)N(C)c2cccc(C)c2-c2ccc(-c3nnn[nH]3)s2)s1] | methyl 5-((2-(5-(1H-tetrazol-5-yl)thiophen- 2-yl)-3- methylphenyl)(methyl)carbamoyl)thiophene- 2-carboxylate |
| RU-NT- 444 | 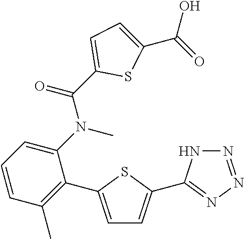 [SMILES: Cc1cccc(N(C)C(=O)c2ccc(C(=O)O)s2)c1-c1ccc(-c2nnn[nH]2)s1] | 5-((2-(5-(1H-tetrazol-5-yl)thiophen-2-yl)-3- methylphenyl)(methyl)carbamoyl)thiophene- 2-carboxylic acid |
| RU-NT- 445 | 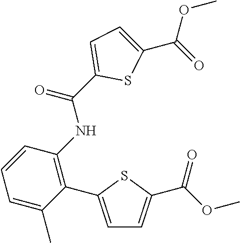 [SMILES: COC(=O)c1ccc(C(=O)Nc2cccc(C)c2-c2ccc(C(=O)OC)s2)s1] | methyl 5-((2-(5-(methoxycarbonyl)thiophen- 2-yl)-3-methylphenyl)carbamoyl)thiophene- 2-carboxylate |
| RU-NT- 446 | 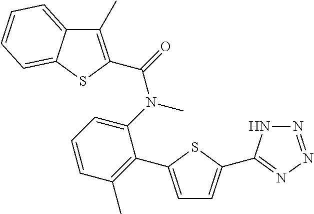 [SMILES: Cc1cccc(N(C)C(=O)c2sc3ccccc3c2C)c1-c1ccc(-c2nnn[nH]2)s1] | N-(2-(5-(1H-tetrazol-5-yl)thiophen-2-yl)-3- methylphenyl)-N,3- dimethylbenzo[b]thiophene-2-carboxamide |
| RU-NT- 447 | 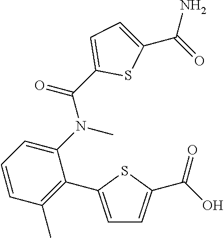 [SMILES: Cc1cccc(N(C)C(=O)c2ccc(C(N)=O)s2)c1-c1ccc(C(=O)O)s1] | [symbol] [symbol] [symbol] |
| RU-NT- 448 | 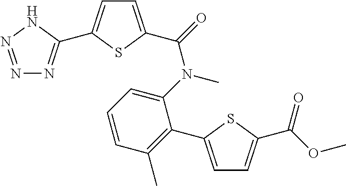 [SMILES: COC(=O)c1ccc(-c2c(C)cccc2N(C)C(=O)c2ccc(-c3nnn[nH]3)s2)s1] | methyl 5-(2-methyl-6-(N-methyl-5-(1H- tetrazol-5-yl)thiophene-2- carboxamido)phenyl)thiophene-2- carboxylate |
| RU-NT- 449 | 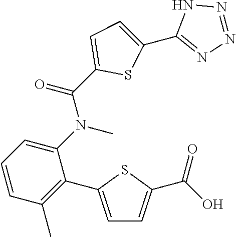 [SMILES: Cc1cccc(N(C)C(=O)c2ccc(-c3nnn[nH]3)s2)c1-c1ccc(C(=O)O)s1] | 5-(2-methyl-6-(N-methyl-5-(1H-tetrazol-5- yl)thiophene-2- carboxamido)phenyl)thiophene-2-carboxylic acid |
| RU-NT- 450 | 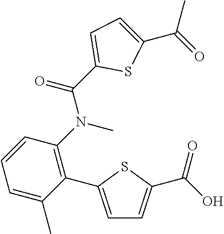 [SMILES: CC(=O)c1ccc(C(=O)N(C)c2cccc(C)c2-c2ccc(C(=O)O)s2)s1] | 5-(2-(5-acetyl-N-methylthiophene-2- carboxamido)-6-methylphenyl)thiophene-2- carboxylic acid |
| RU-NT- 451 | 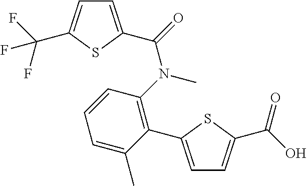 [SMILES: Cc1cccc(N(C)C(=O)c2ccc(C(F)(F)F)s2)c1-c1ccc(C(=O)O)s1] | 5-(2-methyl-6-(N-methyl-5- (trifluoromethyl)thiophene-2- carboxamido)phenyl)thiophene-2-carboxylic acid |
| RU-NT- 452 | 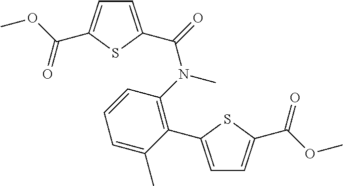 | methyl 5-(2-(5-(methoxycarbonyl)-N- methylthiophene-2-carboxamido)-6- methylphenyl)thiophene-2-carboxylate |
| RU-NT- 453 | 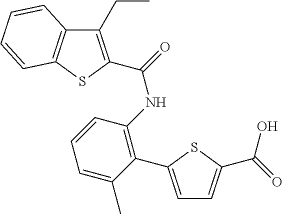 [SMILES: CCc1c(C(=O)Nc2cccc(C)c2-c2ccc(C(=O)O)s2)sc2ccccc12] | 5-(2-(3-ethylbenzo[b]thiophene-2- carboxamido)-6-methylphenyl)thiophene-2- carboxylic acid |
| RU-NT- 454 | 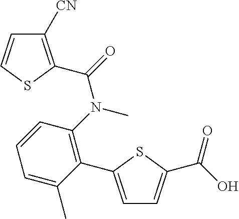 [SMILES: Cc1cccc(N(C)C(=O)c2sccc2C#N)c1-c1ccc(C(=O)O)s1] | 5-(2-(3-cyano-N-methylthiophene-2- carboxamido)-6-methylphenyl)thiophene-2- carboxylic acid |
| RU-NT- 455 | 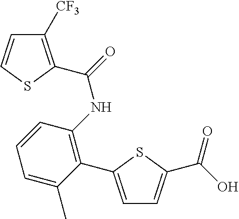 [SMILES: Cc1cccc(NC(=O)c2sccc2C(F)(F)F)c1-c1ccc(C(=O)O)s1] | 5-(2-methyl-6-(3- (trifluoromethyl)thiophene-2- carboxamido)phenyl)thiophene-2-carboxylic acid |
| RU-NT- 456 | 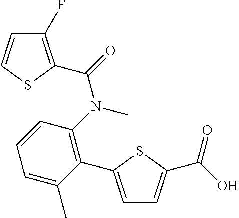 [SMILES: Cc1cccc(N(C)C(=O)c2sccc2F)c1-c1ccc(C(=O)O)s1] | 5-(2-(3-fluoro-N-methylthiophene-2- carboxamido)-6-methylphenyl)thiophene-2- carboxylic acid |
| RU-NT- 457 | 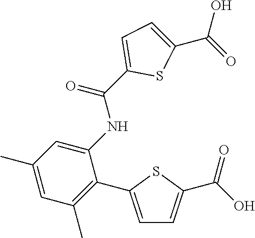 [SMILES: Cc1cc(C)c(-c2ccc(C(=O)O)s2)c(NC(=O)c2ccc(C(=O)O)s2)c1] | 5-((2-(5-carboxythiophen-2-yl)-3,5- dimethylphenyl)carbamoyl)thiophene-2- carboxylic acid |
| RU-NT- 458 | 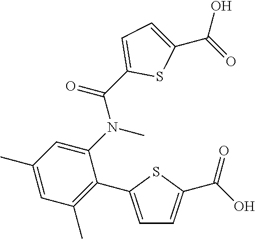 [SMILES: Cc1cc(C)c(-c2ccc(C(=O)O)s2)c(N(C)C(=O)c2ccc(C(=O)O)s2)c1] | 5-(2-(5-carboxy-N-methylthiophene-2- carboxamido)-4,6- dimethylphenyl)thiophene-2-carboxylic acid |
| RU-NT- 459 | 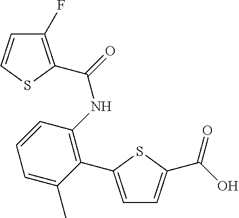 [SMILES: Cc1cccc(NC(=O)c2sccc2F)c1-c1ccc(C(=O)O)s1] | 5-(2-(3-fluorothiophene-2-carboxamido)-6- methylphenyl)thiophene-2-carboxylic acid |
| RU-NT- 460 | 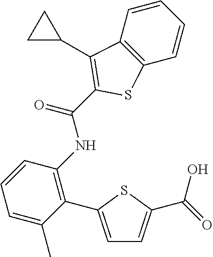 [SMILES: Cc1cccc(NC(=O)c2sc3ccccc3c2C2CC2)c1-c1ccc(C(=O)O)s1] | 5-(2-(3-cyclopropylbenzo[b]thiophene-2- carboxamido)-6-methylphenyl)thiophene-2- carboxylic acid |
| RU-NT- 461 | 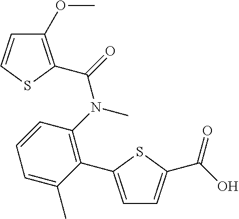 [SMILES: COc1ccsc1C(=O)N(C)c1cccc(C)c1-c1ccc(C(=O)O)s1] | 5-(2-(3-methoxy-N-methylthiophene-2- carboxamido)-6-methylphenyl)thiophene-2- carboxylic acid |
| RU-NT- 462 | 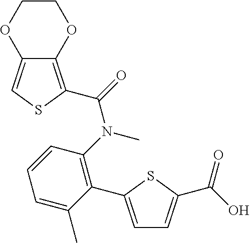 [SMILES: Cc1cccc(N(C)C(=O)c2scc3c2OCCO3)c1-c1ccc(C(=O)O)s1] | 5-(2-methyl-6-(N-methyl-2,3- dihydrothieno[3,4-b][1,4]dioxine-5- carboxamido)phenyl)thiophene-2-carboxylic acid |
| RU-NT- 463 | 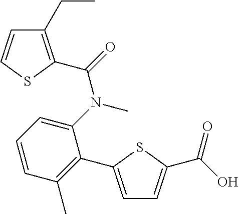 [SMILES: CCc1ccsc1C(=O)N(C)c1cccc(C)c1-c1ccc(C(=O)O)s1] | 5-(2-(3-ethyl-N-methylthiophene-2- carboxamido)-6-methylphenyl)thiophene-2- carboxylic acid |
| RU-NT- 464 | 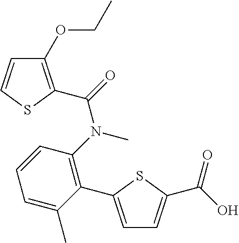 [SMILES: CCOc1ccsc1C(=O)N(C)c1cccc(C)c1-c1ccc(C(=O)O)s1] | 5-(2-(3-ethoxy-N-methylthiophene-2- carboxamido)-6-methylphenyl)thiophene-2- carboxylic acid |
| RU-NT- 465 | 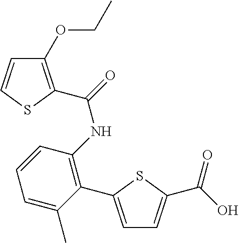 [SMILES: CCOc1ccsc1C(=O)Nc1cccc(C)c1-c1ccc(C(=O)O)s1] | 5-(2-(3-ethoxythiophene-2-carboxamido)-6- methylphenyl)thiophene-2-carboxylic acid |
| RU-NT- 466 | 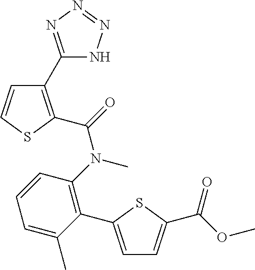 [SMILES: COC(=O)c1ccc(-c2c(C)cccc2N(C)C(=O)c2sccc2-c2nnn[nH]2)s1] | methyl 5-(2-methyl-6-(N-methyl-3-(1H- tetrazol-5-yl)thiophene-2- carboxamido)phenyl)thiophene-2- carboxylate |
| RU-NT- 467 | 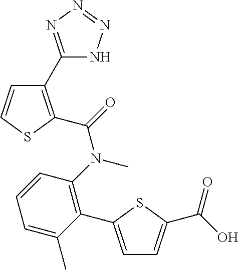 [SMILES: Cc1cccc(N(C)C(=O)c2sccc2-c2nnn[nH]2)c1-c1ccc(C(=O)O)s1] | 5-(2-methyl-6-(N-methyl-3-(1H-tetrazol-5- yl)thiophene-2- carboxamido)phenyl)thiophene-2-carboxylic acid |
| RU-NT- 468 | 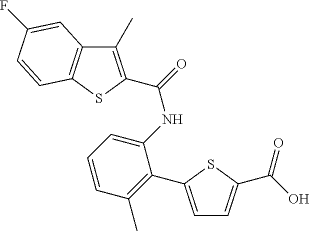 [SMILES: Cc1cccc(NC(=O)c2sc3ccc(F)cc3c2C)c1-c1ccc(C(=O)O)s1] | 5-(2-(5-fluoro-3-methylbenzo[b]thiophene- 2-carboxamido)-6-methylphenyl)thiophene- 2-carboxylic acid |
| RU-NT- 469 | 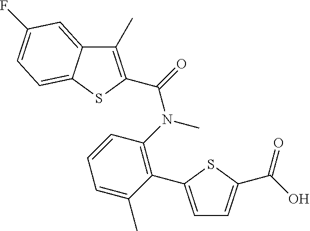 [SMILES: Cc1cccc(N(C)C(=O)c2sc3ccc(F)cc3c2C)c1-c1ccc(C(=O)O)s1] | 5-(2-(5-fluoro-N,3- dimethylbenzo[b]thiophene-2-carboxamido)- 6-methylphenyl)thiophene-2-carboxylic acid |
| RU-NT- 470 | 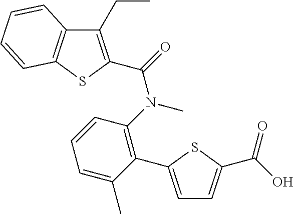 [SMILES: CCc1c(C(=O)N(C)c2cccc(C)c2-c2ccc(C(=O)O)s2)sc2ccccc12] | 5-(2-(3-ethyl-N-methylbenzo[b]thiophene-2- carboxamido)-6-methylphenyl)thiophene-2- carboxylic acid |
| RU-NT- 471 | 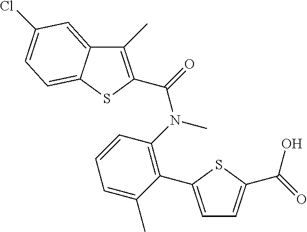 [SMILES: Cc1cccc(N(C)C(=O)c2sc3ccc(Cl)cc3c2C)c1-c1ccc(C(=O)O)s1] | 5-(2-(5-chloro-N,3- dimethylbenzo[b]thiophene-2-carboxamido)- 6-methylphenyl)thiophene-2-carboxylic acid |
| RU-NT- 472 | 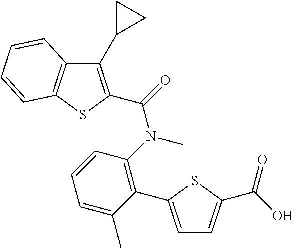 [SMILES: Cc1cccc(N(C)C(=O)c2sc3ccccc3c2C2CC2)c1-c1ccc(C(=O)O)s1] | 5-(2-(3-cyclopropyl-N- methylbenzo[b]thiophene-2-carboxamido)- 6-methylphenyl)thiophene-2-carboxylic acid |
| RU-NT- 473 | 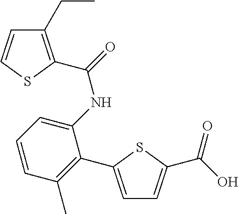 [SMILES: CCc1ccsc1C(=O)Nc1cccc(C)c1-c1ccc(C(=O)O)s1] | 5-(2-(3-ethylthiophene-2-carboxamido)-6- methylphenyl)thiophene-2-carboxylic acid |
| RU-NT- 474 | 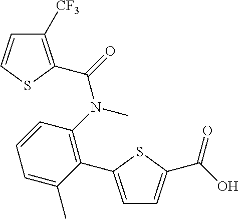 [SMILES: Cc1cccc(N(C)C(=O)c2sccc2C(F)(F)F)c1-c1ccc(C(=O)O)s1] | 5-(2-methyl-6-(N-methyl-3- (trifluoromethyl)thiophene-2- carboxamido)phenyl)thiophene-2-carboxylic acid |
| RU-NT- 475 | 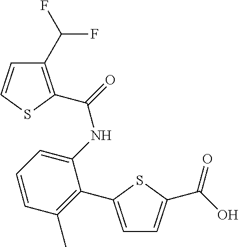 [SMILES: Cc1cccc(NC(=O)c2sccc2C(F)F)c1-c1ccc(C(=O)O)s1] | 5-(2-(3-(difluoromethyl)thiophene-2- carboxamido)-6-methylphenyl)thiophene-2- carboxylic acid |
| RU-NT- 476 | 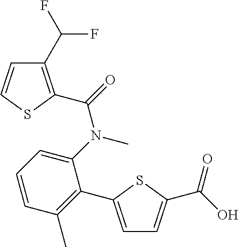 [SMILES: Cc1cccc(N(C)C(=O)c2sccc2C(F)F)c1-c1ccc(C(=O)O)s1] | 5-(2-(3-(difluoromethyl)-N- methylthiophene-2-carboxamido)-6- methylphenyl)thiophene-2-carboxylic acid |
| RU-NT- 477 | 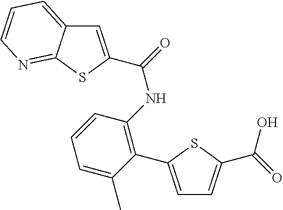 [SMILES: Cc1cccc(NC(=O)c2cc3cccnc3s2)c1-c1ccc(C(=O)O)s1] | 5-(2-methyl-6-(thieno[2,3-b]pyridine-2- carboxamido)phenyl)thiophene-2-carboxylic acid |
| RU-NT- 478 | 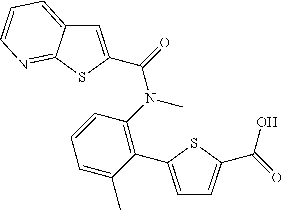 [SMILES: Cc1cccc(N(C)C(=O)c2cc3cccnc3s2)c1-c1ccc(C(=O)O)s1] | 5-(2-methyl-6-(N-methylthieno[2,3- b]pyridine-2- carboxamido)phenyl)thiophene-2-carboxylic acid |
| RU-NT- 479 | 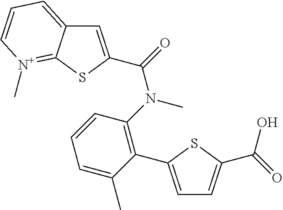 [SMILES: Cc1cccc(N(C)C(=O)c2cc3ccc[n+](C)c3s2)c1-c1ccc(C(=O)O)s1] | 2-((2-(5-carboxythiophen-2-yl)-3- methylphenyl)(methyl)carbamoyl)-7- methylthieno[2,3-b]pyridin-7-ium |
| RU-NT- 480 | 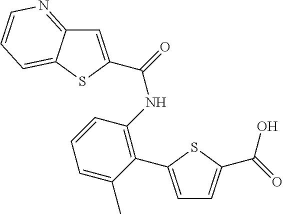 [SMILES: Cc1cccc(NC(=O)c2cc3ncccc3s2)c1-c1ccc(C(=O)O)s1] | 5-(2-methyl-6-(thieno[3,2-b]pyridine-2- carboxamido)phenyl)thiophene-2-carboxylic acid |
| RU-NT- 481 | 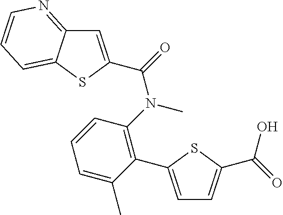 [SMILES: Cc1cccc(N(C)C(=O)c2cc3ncccc3s2)c1-c1ccc(C(=O)O)s1] | 5-(2-methyl-6-(N-methylthieno[3,2- b]pyridine-2- carboxamido)phenyl)thiophene-2-carboxylic acid |
| RU-NT- 482 | 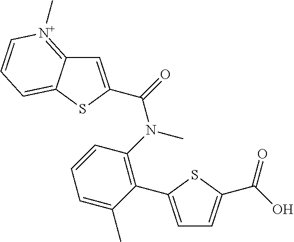 [SMILES: Cc1cccc(N(C)C(=O)c2cc3c(ccc[n+]3C)s2)c1-c1ccc(C(=O)O)s1] | 2-((2-(5-carboxythiophen-2-yl)-3- methylphenyl)(methyl)carbamoyl)-4- methylthieno[3,2-b]pyridin-4-ium |
| RU-NT- 485 | 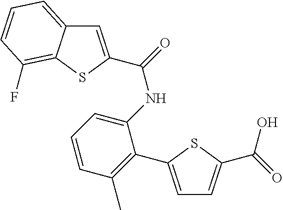 [SMILES: Cc1cccc(NC(=O)c2cc3cccc(F)c3s2)c1-c1ccc(C(=O)O)s1] | 5-(2-(7-fluorobenzo[b]thiophene-2- carboxamido)-6-methylphenyl)thiophene-2- carboxylic acid |
| RU-NT- 486 | 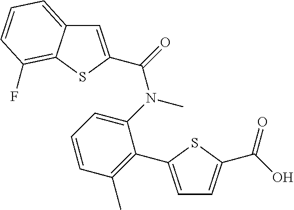 [SMILES: Cc1cccc(N(C)C(=O)c2cc3cccc(F)c3s2)c1-c1ccc(C(=O)O)s1] | 5-(2-(7-fluoro-N-methylbenzo[b]thiophene- 2-carboxamido)-6-methylphenyl)thiophene- 2-carboxylic acid |
| RU-NT- 487 | 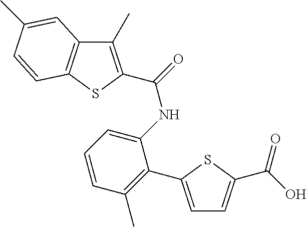 [SMILES: Cc1ccc2sc(C(=O)Nc3cccc(C)c3-c3ccc(C(=O)O)s3)c(C)c2c1] | 5-(2-(3,5-dimethylbenzo[b]thiophene-2- carboxamido)-6-methylphenyl)thiophene-2- carboxylic acid |
| RU-NT- 488 | 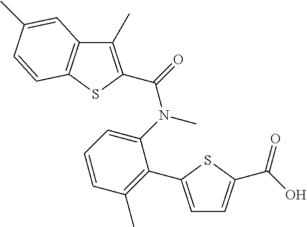 [SMILES: Cc1ccc2sc(C(=O)N(C)c3cccc(C)c3-c3ccc(C(=O)O)s3)c(C)c2c1] | 5-(2-methyl-6-(N,3,5- trimethylbenzo[b]thiophene-2- carboxamido)phenyl)thiophene-2-carboxylic acid |
| RU-NT- 489 | 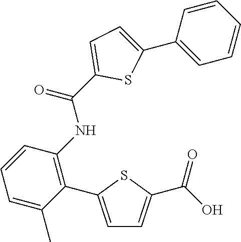 [SMILES: Cc1cccc(NC(=O)c2ccc(-c3ccccc3)s2)c1-c1ccc(C(=O)O)s1] | 5-(2-methyl-6-(5-phenylthiophene-2- carboxamido)phenyl)thiophene-2-carboxylic acid |
| RU-NT- 490 | 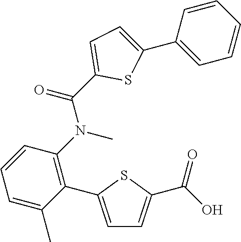 [SMILES: Cc1cccc(N(C)C(=O)c2ccc(-c3ccccc3)s2)c1-c1ccc(C(=O)O)s1] | 5-(2-methyl-6-(N-methyl-5- phenylthiophene-2- carboxamido)phenyl)thiophene-2-carboxylic acid |
| RU-NT- 492 | 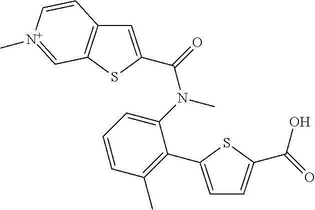 [SMILES: Cc1cccc(N(C)C(=O)c2cc3cc[n+](C)cc3s2)c1-c1ccc(C(=O)O)s1] | 2-((2-(5-carboxythiophen-2-yl)-3- methylphenyl)(methyl)carbamoyl)-6- methylthieno[2,3-c]pyridin-6-ium |
| RU-NT- 493 | 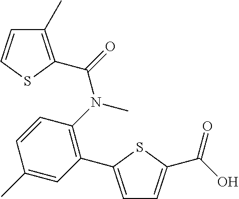 [SMILES: Cc1ccc(N(C)C(=O)c2sccc2C)c(-c2ccc(C(=O)O)s2)c1] | 5-(2-(N,3-dimethylthiophene-2- carboxamido)-5-methylphenyl)thiophene-2- carboxylic acid |
| RU-NT- 494 | 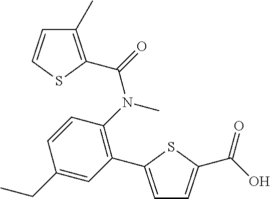 [SMILES: CCc1ccc(N(C)C(=O)c2sccc2C)c(-c2ccc(C(=O)O)s2)c1] | 5-(2-(N,3-dimethylthiophene-2- carboxamido)-5-ethylphenyl)thiophene-2- carboxylic acid |
| RU-NT- 495 | 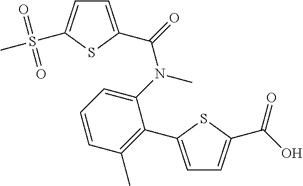 [SMILES: Cc1cccc(N(C)C(=O)c2ccc(S(C)(=O)=O)s2)c1-c1ccc(C(=O)O)s1] | 5-(2-methyl-6-(N-methyl-5- (methylsulfonyl)thiophene-2- carboxamido)phenyl)thiophene-2-carboxylic acid |
| RU-NT- 496 | 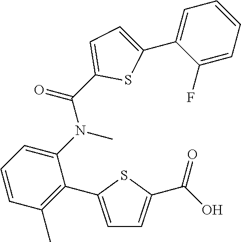 [SMILES: Cc1cccc(N(C)C(=O)c2ccc(-c3ccccc3F)s2)c1-c1ccc(C(=O)O)s1] | 5-(2-(5-(2-fluorophenyl)-N- methylthiophene-2-carboxamido)-6- methylphenyl)thiophene-2-carboxylic acid |
| RU-NT- 499 | 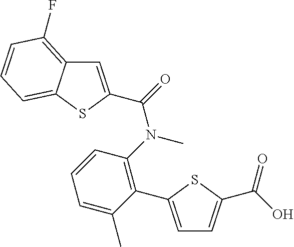 [SMILES: Cc1cccc(N(C)C(=O)c2cc3c(F)cccc3s2)c1-c1ccc(C(=O)O)s1] | 5-(2-(4-fluoro-N-methylbenzo[b]thiophene- 2-carboxamido)-6-methylphenyl)thiophene- 2-carboxylic acid |
| RU-NT- 500 | 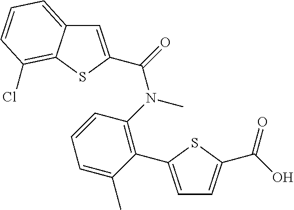 [SMILES: Cc1cccc(N(C)C(=O)c2cc3cccc(Cl)c3s2)c1-c1ccc(C(=O)O)s1] | 5-(2-(7-chloro-N-methylbenzo[b]thiophene- 2-carboxamido)-6-methylphenyl)thiophene- 2-carboxylic acid |
| RU-NT- 501 | 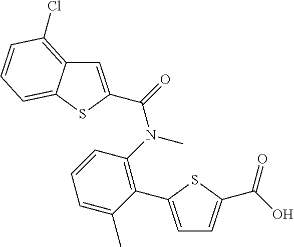 [SMILES: Cc1cccc(N(C)C(=O)c2cc3c(Cl)cccc3s2)c1-c1ccc(C(=O)O)s1] | 5-(2-(4-chloro-N-methylbenzo[b]thiophene- 2-carboxamido)-6-methylphenyl)thiophene- 2-carboxylic acid |
| RU-NT- 502 | 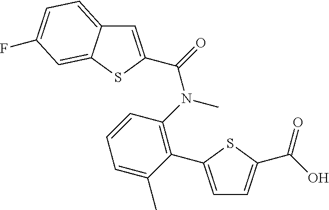 [SMILES: Cc1cccc(N(C)C(=O)c2cc3ccc(F)cc3s2)c1-c1ccc(C(=O)O)s1] | 5-(2-(6-fluoro-N-methylbenzo[b]thiophene- 2-carboxamido)-6-methylphenyl)thiophene- 2-carboxylic acid |
| RU-NT- 503 | 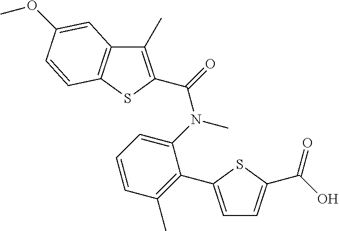 [SMILES: COc1ccc2sc(C(=O)N(C)c3cccc(C)c3-c3ccc(C(=O)O)s3)c(C)c2c1] | 5-(2-(5-methoxy-N,3- dimethylbenzo[b]thiophene-2-carboxamido)- 6-methylphenyl)thiophene-2-carboxylic acid |
| RU-NT- 504 | 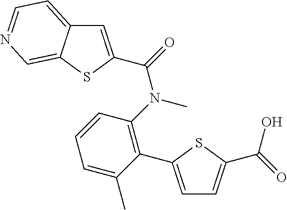 [SMILES: Cc1cccc(N(C)C(=O)c2cc3ccncc3s2)c1-c1ccc(C(=O)O)s1] | 5-(2-methyl-6-(N-methylthieno[2,3- c]pyridine-2- carboxamido)phenyl)thiophene-2-carboxylic acid |
| RU-NT- 505 | 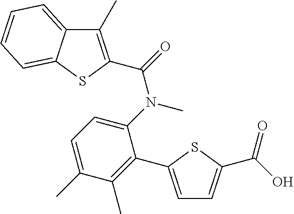 [SMILES: Cc1ccc(N(C)C(=O)c2sc3ccccc3c2C)c(-c2ccc(C(=O)O)s2)c1C] | 5-(6-(N,3-dimethylbenzo[b]thiophene-2- carboxamido)-2,3- dimethylphenyl)thiophene-2-carboxylic acid |
| RU-NT- 506 | 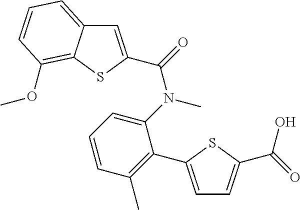 [SMILES: COc1cccc2cc(C(=O)N(C)c3cccc(C)c3-c3ccc(C(=O)O)s3)sc12] | 5-(2-(7-methoxy-N- methylbenzo[b]thiophene-2-carboxamido)- 6-methylphenyl)thiophene-2-carboxylic acid |
| RU-NT- 507 | 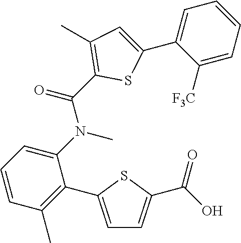 [SMILES: Cc1cc(-c2ccccc2C(F)(F)F)sc1C(=O)N(C)c1cccc(C)c1-c1ccc(C(=O)O)s1] | 5-(2-(N,3-dimethyl-5-(2- (trifluoromethyl)phenyl)thiophene-2- carboxamido)-6-methylphenyl)thiophene-2- carboxylic acid |

### Methods

### Therapeutic Methods

In another aspect the disclosure provides a method of treating, preventing, and/or ameliorating toxicity caused by a ribosome inactivating protein (RIP) in a subject, the method comprising administering to the subject a therapeutically effective amount of a compound of Formula (I), Formula (II), and/or Formula (III), or a pharmaceutically acceptable salt thereof.

In certain embodiments, the compound of Formula (III) comprises:

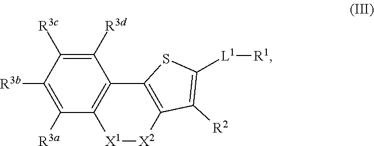

wherein:
   R¹ is a a carboxylic acid (—COOH), —C(═O)C(OH)₂C(═O)OR^{A}, a carboxylic acid bioisostere, or a carboxylic acid precursor;
   R² is H;
   R^{3a}, R^{3b}, R^{3b}, and R^{3d} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   L¹ is selected from the group consisting of a bond and optionally substituted C₁-C₂ alkylenyl;
   X¹ is selected from the group consisting of —C(R^{4a})(R^{4b})—, O, and S;
   X² is selected from the group consisting of —C(R^{5a})(R^{5b})— and a bond;
   R^{4a} and R^{4b} are each independently selected from the group consisting of H and optionally substituted C₁-C₆ alkyl,
      wherein no more than one of R^{4a} and R^{4b} is H;
   R^{5a} and R^{5b} are each independently selected from the group consisting of H and optionally substituted C₁-C₆ alkyl,
      or R^{5a} and R^{5b} can combine with the carbon atom to which they are bound to form a carbonyl moiety (C═O); and
   R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl.

In certain embodiments, the RIP is ricin or Shiga toxin 2a (Stx2a).

In certain embodiments, the compound inhibitions depurination activity of the RIP. In certain embodiments, the compound inhibits interaction of the RIP with a ribosome. In certain embodiments, the compound inhibits the interaction of an active A chain (RTA) of the RIP with a ribosome. In certain embodiments, the compound binds to the ribosome binding site of the RTA. In certain embodiments, the ribosome inactivating protein (RIP) is either a type I or type II RIP.

In certain embodiments, the compound is administered as a pharmaceutical composition to the subject.

In certain embodiments, the subject is administered at least one additional agent useful for treating, ameliorating, and/or preventing the toxicity caused by RIP. In certain embodiments, the at least one additional agent is selected from the group consisting of immunotherapeutics and vaccines. In certain embodiments, administering the compound to the subject allows for administering a lower dose of the at least one additional agent as compared to the dose of the at least one additional agent alone that is required to achieve similar results in treating, ameliorating, and/or preventing toxicity caused by RIP. In certain embodiments, the compound and the at least one additional agent are co-administered to the subject. In certain embodiments, the compound and the at least one additional agent are co-formulated.

In certain embodiments, the compound of Formula (I) is a compound of Formula (III).

In certain embodiments, the compound of Formula (I), (II) or (III) is selected from the group consisting of:
8-chloro-4H-thieno[3,2-c]thiochromene-2-carbohydrazide;
methyl 8-fluoro-4H-thieno[3,2-c]chromene-2-carboxylate;
1-(8-chloro-4H-thieno[3,2-c]thiochromen-2-yl)ethan-1-one;
8-chloro-4H-thieno[3,2-c]thiochromene-2-carboxylic acid;
4H-thieno[3,2-c]chromene-2-carboxylic acid;
8-fluoro-4H-thieno[3,2-c]thiochromene-2-carboxylic acid;
benzo[b]thieno[2,3-d]thiophene-2-carboxylic acid;
5,5-dimethyl-4,5-dihydronaphtho[1,2-b]thiophene-2-carboxylic acid;
5-(2-ethyl-6-methylphenyl)thiophene-2-carboxylic acid;
5-(2,6-diethylphenyl)thiophene-2-carboxylic acid;
5,5-dimethyl-4,5-dihydronaphtho[1,2-b]thiophene-2-carboxamide;
5,5-dimethyl-4,5-dihydronaphtho[1,2-b]thiophene-2-carbonitrile;
5-(5,5-dimethyl-4,5-dihydronaphtho[1,2-b]thiophen-2-yl)-2H-tetrazole; and
5,5-dimethyl-N-(methylsulfonyl)-4,5-dihydronaphtho[1,2-b]thiophene-2-carboxamide.

In certain embodiments, the subject is a mammal.

In certain embodiments, the subject is a human.

### Fluorescence Polarization Competition Assay

In another aspect, the disclosure provides a method for measuring binding affinity of a small molecule for an active A chain (RTA) of a ribosome inactivating protein (RIP). In certain embodiments, the method comprises contacting a small molecule with a mixture comprising a fluorescently labeled P11 polypeptide (SEQ ID NO:1) and RTA to provide a displacing mixture.

In certain embodiments, the method comprises measuring fluorescence of the displacing mixture to provide a displaced fluorescence mixture.

In certain embodiments, the method comprises measuring fluorescence of a control sample comprising the fluorescently labeled P11 polypeptide (SEQ ID NO:1) and RTA, and lacking the small molecule, to provide a control measurement.

In certain embodiments, the method comprises comparing the displaced fluorescence measurement and the control fluorescence measurement.

In certain embodiments, more than one occurrence of each of steps (a)-(d) is performed. In certain embodiments, each series of steps (a)-(d) represents a single experiment performed in a series of parallel experiments. In certain embodiments, each independent occurrence of steps (a)-(d) is performed in a well of a microplate, optionally wherein the microplate comprises a 96-well microplate.

In certain embodiments, each occurrence of steps (a)-(d) independently occurs with a concentration of the small molecule ranging from about 1 nM to about 1000 μM.

In certain embodiments, each occurrence of steps (a)-(d) occurs with a concentration of the small molecule is selected from the group consisting of about 0.001, about 0.01, about 0.1, about 1, about 5, about 10, about 20, about 40, about 80, about 125, about 160, about 250, about 500, or about 1000 μM.

In certain embodiments, the RTA and fluorescently labeled P11 polypeptide (SEQ ID NO:1) have a molar ratio of about 3:1. In certain embodiments, the RTA has a concentration of about 3 μM and the fluorescently labeled P11 polypeptide (SEQ ID NO:1) has a concentration of about 1 μM.

In certain embodiments, the fluorescent label comprises a BODIPY dye. In certain embodiments, the BODIPY dye comprises and/or is prepared using a BODIPY TMR-X N-hydroxysuccinimide ester:

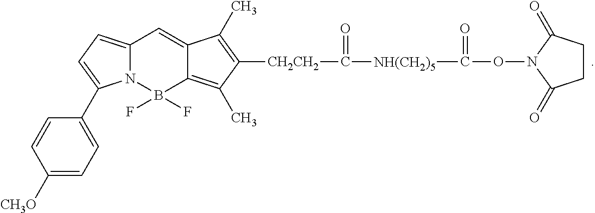

In certain embodiments, the contacting step further comprises at least one selected from the group consisting of centrifuging the displacing mixture one or more times and incubating the displacing mixture without significant light exposure.

In certain embodiments, each measuring step comprises use of an excitation filter of about 530/25 nm and an emission filter of about 590/35 nm.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

The use of antibodies to deliver therapeutic proteins in the form of antibody drug conjugates is a technology to deliver drugs to specific tissues. For example, an antibody to the AMHR2 could be used to deliver an ACVR1 inhibitor to ovarian cells or to disease tissue (e.g., endometriosis tissue) or to cancers (e.g., ovarian cancer) with expression or overexpression of AMHR2. In this context, an antibody bound to the cell surface AMHR2 transmembrane receptor is internalized as a consequence of the constitutive recycling of these type 2 receptors. In the same method, antibodies that specifically target ACVR2A or ACVR2B or BMPR2 could be used to achieve cell-specific delivery of ACVR1 inhibitors to avoid systemic exposure to this inhibitor when cell-specific delivery is the preferred therapeutic objective. The antibody form utilized can represent full length or modified immunoglobulin designs incorporating IgG1, IgG4 or fragments referred to as single chain Fv (scFv) or diabody or similar modifications of an antibody that are specifically utilized for targeted delivery of small molecules without the need for antibody dependent complement cascade (ADCC) or antibody dependent phagocytosis cascade (ADPC).

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors. The skilled artisan is similarly able to determine appropriate dosages for antibody-drug conjugates, based on the half-life and daily maximum exposure achievable with the antibody, or antibody fragments, selected for targeted delivery of an ACVR1 inhibitor.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Materials and Methods

### General

The reagents were purchased and used without additional purification. LC-MS was performed on an Agilent 1100 system with a Waters Micromass ZQ spectrometer using a 5 μL injection on an XBridge C18 (3.5 μM, 4.6×50 mm) column at a temperature of 40° C. with a 4 min gradient from 5% A to 95% B (neutral method: solvent A: 10 mM ammonium formate in water, solvent B: acetonitrile; acidic method: solvent A: 0.1% v/v formic acid in water, solvent B: acetonitrile) at a flow rate of 2 mL/min. The detection used a diode array scanning from 190 to 600 nm or dual-wavelength detectors at 220 and 254 nm (mass detection cone voltage: 30 V). Alternatively, LC-MS and HPLC analyses were performed using the Shimadzu LCMS-2020 system with Dual Ionization Source (acidic method: solvent A: 0.1% v/v trifluoroacetic acid in water, solvent B: acetonitrile, column at a temperature of 40° C. with a 4 min gradient from 5% A to 95% B.

NMR was obtained on a Varian VNMRS 300 MHz, Bruker Avance Neo 400 MHz, Varian VNMRS 500 MHz, or Bruker Avance Neo 500 MHz in DMSO-d₆ (¹H: δ2.50). Flash column chromatography purifications were performed using Biotage Isolera and Selekt systems. Preparative HPLC was performed on ACCQPrep HP125 system, (Waters XBridge BEH C18 column, 100×30 mm×10 μm; mobile phase: A: 0.1% v/v formic acid in water; B: acetonitrile; 10% to 100%, 14 min). The FIRMS analyses were performed using Bruker Apex 7 T FTMS, using an ESI ion source in positive mode. Alternatively, experiments were performed using a Xevo G2-XS QTof mass spectrometer equipped with an Acquity UPLC system. The UPLC-MS system and the column were from Waters Inc. For the LC separation, the following two eluents were used: A containing 0.1% v/v formic acid in water, and B containing neat acetonitrile. Linear gradient (5-100% B in 3 min) was applied. The ionization method was ESI, generating [M+H]+ ions. The mass spectrometer was calibrated using a Leu-enkephalin standard. Capillary exit voltage 330 V. For further HRMS analyses, an Agilent 6546 qTOF mass spectrometer equipped with an Agilent Infinity 1290 UPLC was used. For the latter UPLC the following two eluents were used: A containing 0.1% v/v formic acid in water, and B containing 0.1% v/v formic acid in acetonitrile. Linear gradient (5-100% B in 4 min) was applied. ESI ionization generated M+, [M+H]+, [M+Na]+ or [M+H]+ ions as noted in HRMS tables. The mass spectrometer was calibrated using standard supplied by Agilent Inc. For the UPLC systems a 2.1 mm×50 mm BEH C18 column (particle size 1.7 μm) was utilized. Unless otherwise stated, the purities of the final compounds were equal to or greater than 95% by HPLC analysis. The purities were confirmed with ¹H NMR to look for residual solvents or non-UV active impurities.

### Fluorescence Polarization (FP) Assay

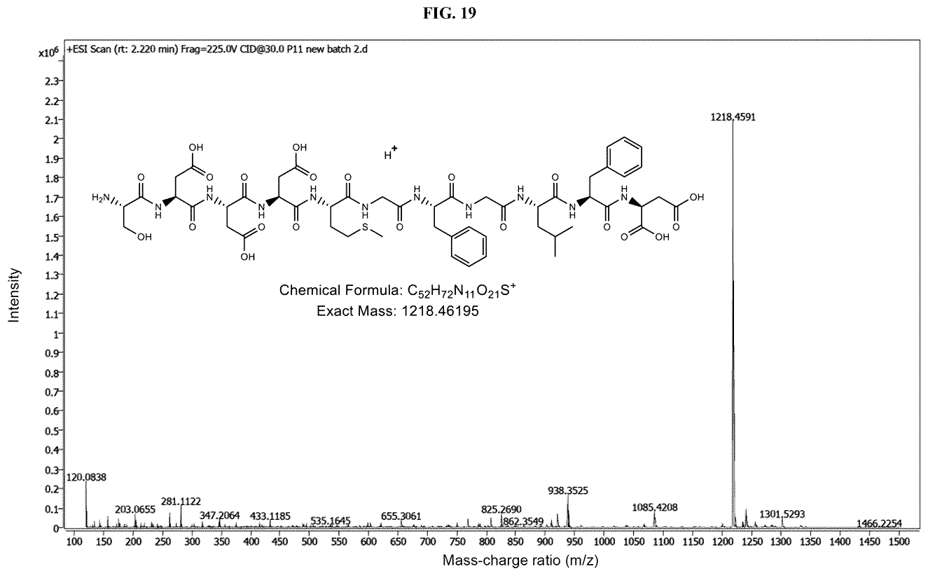

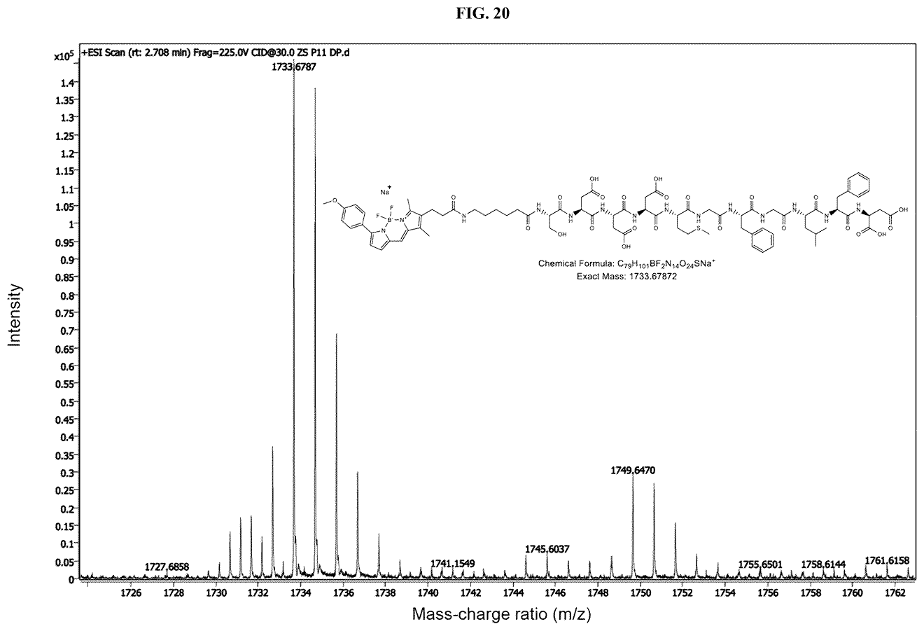

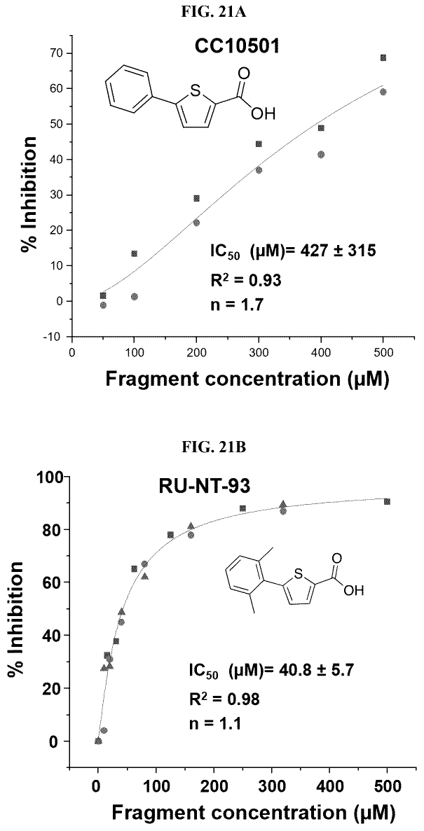

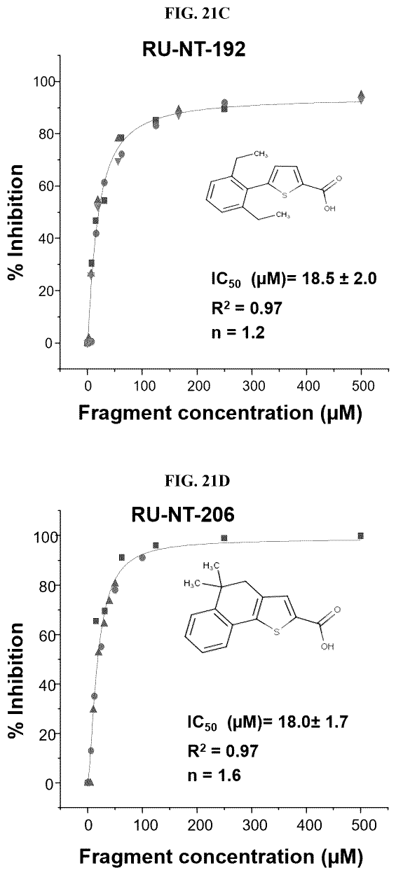

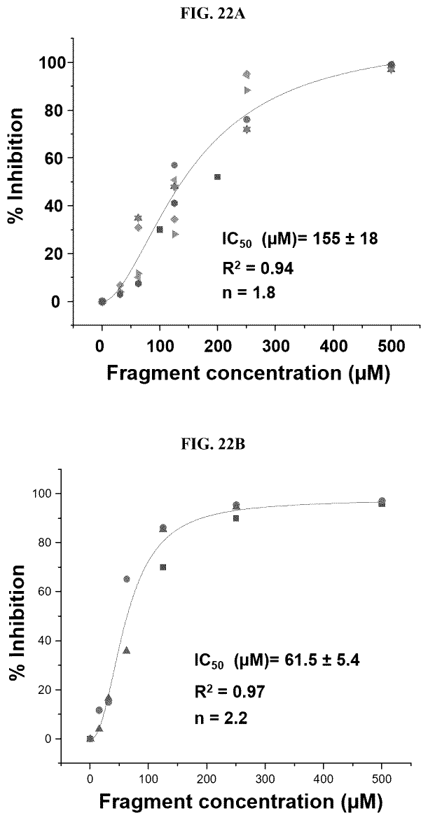

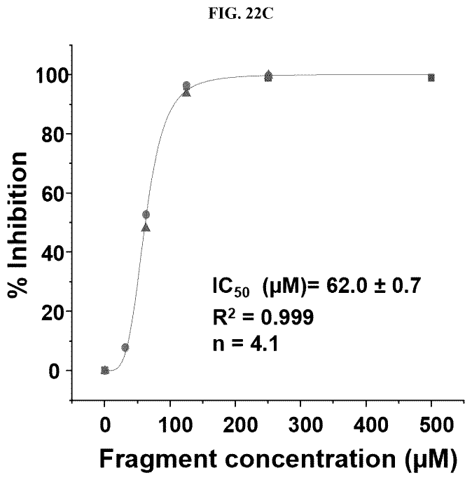

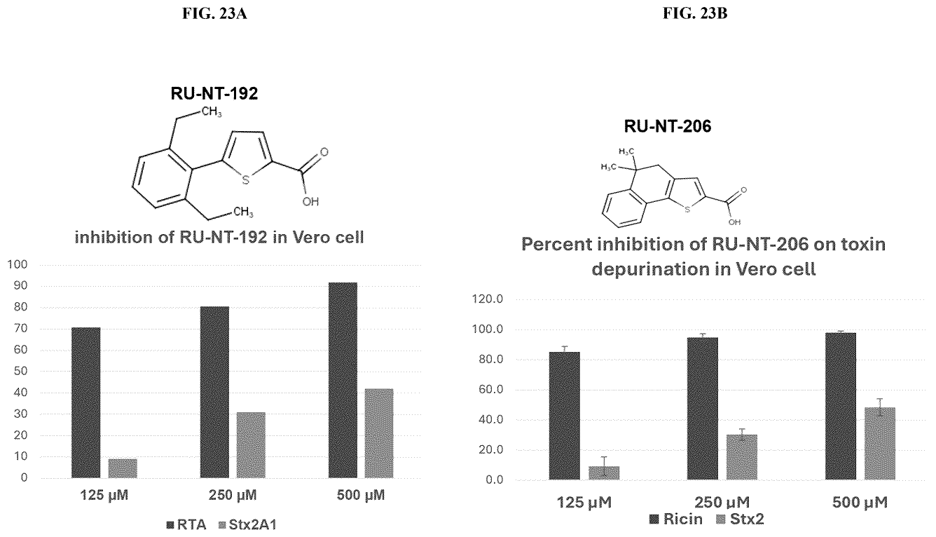

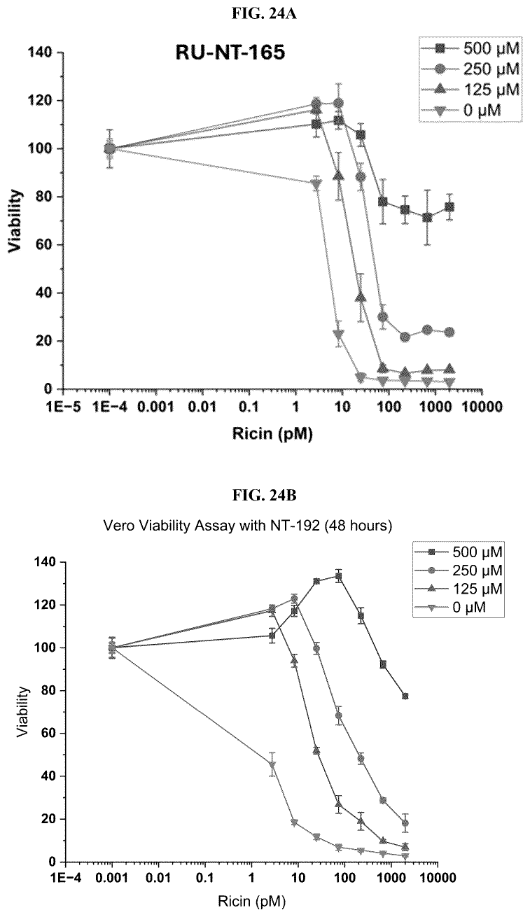

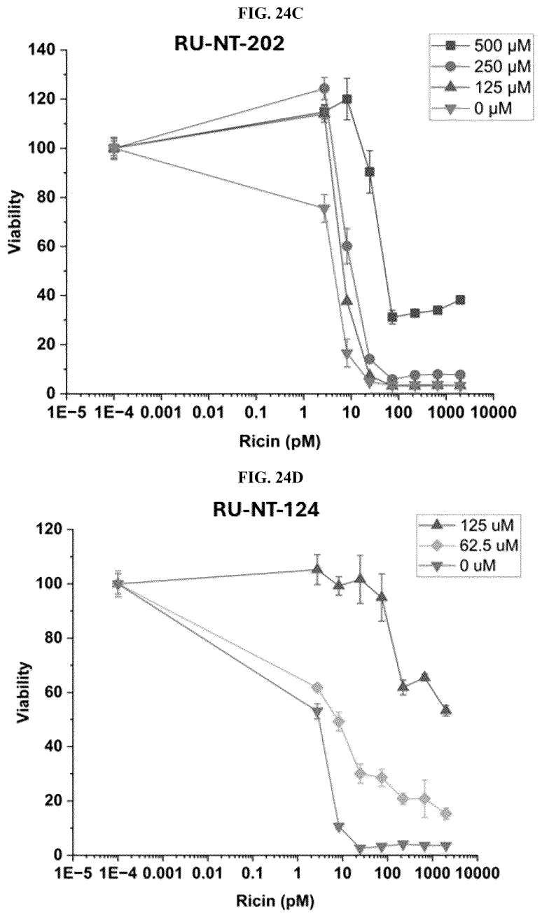

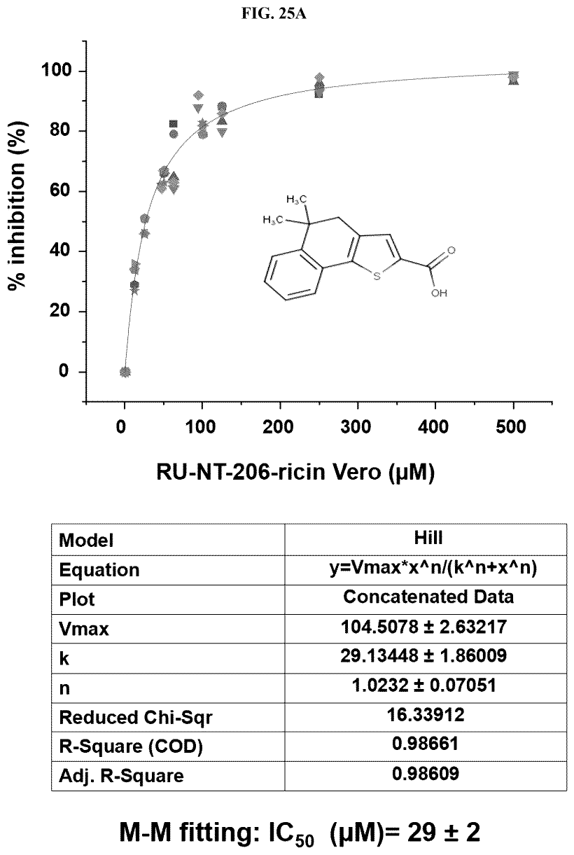

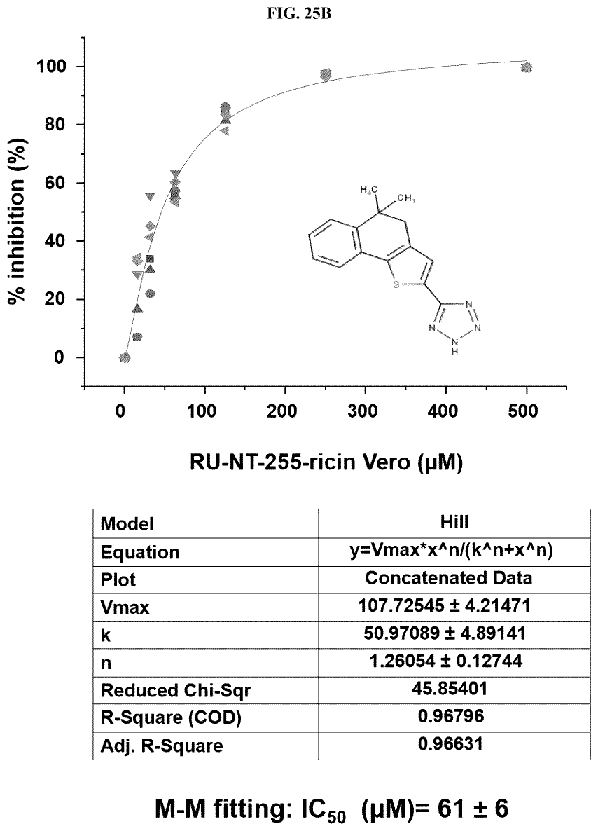

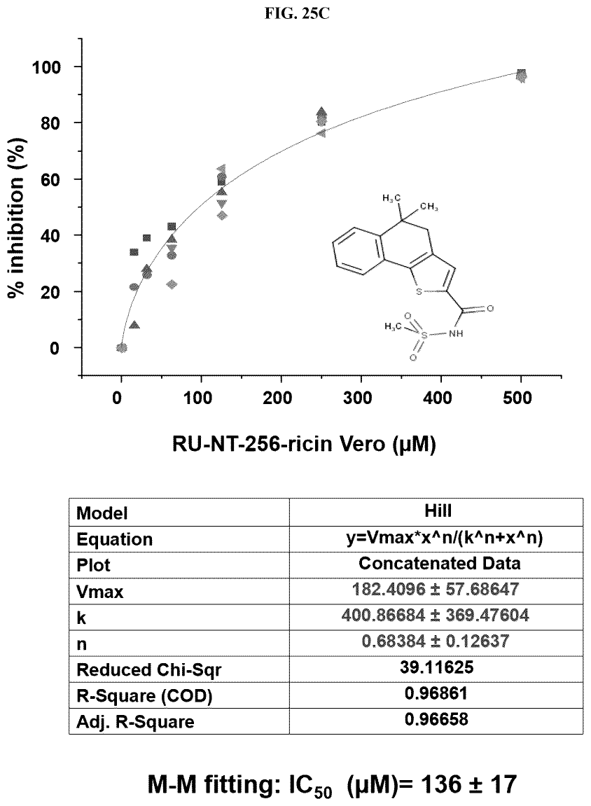

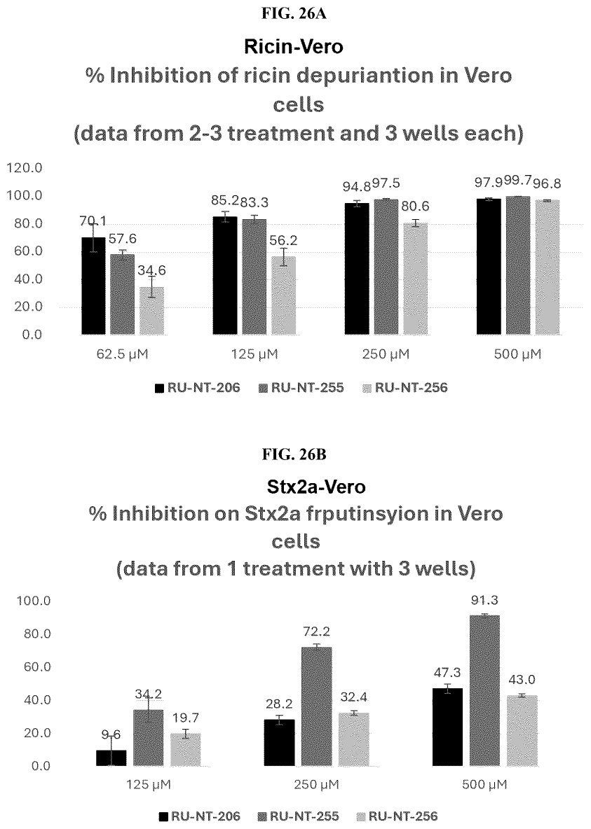

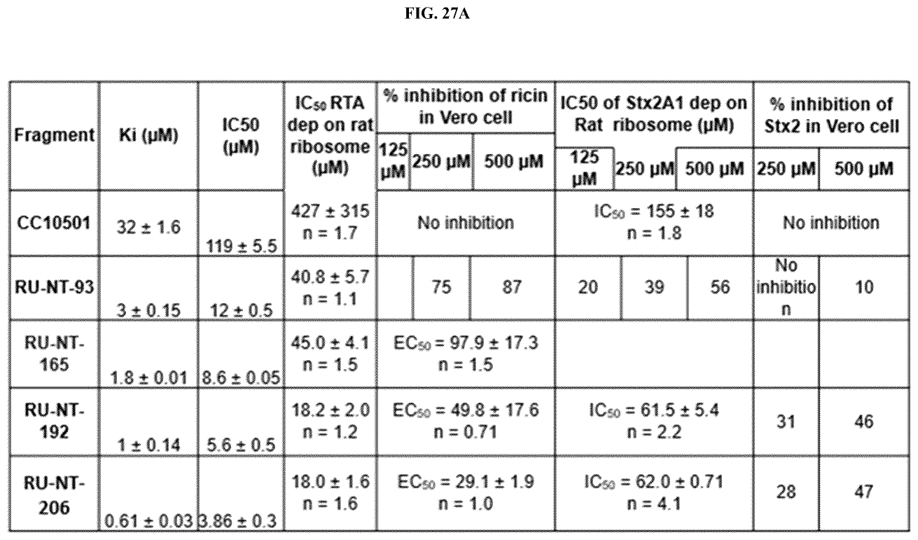

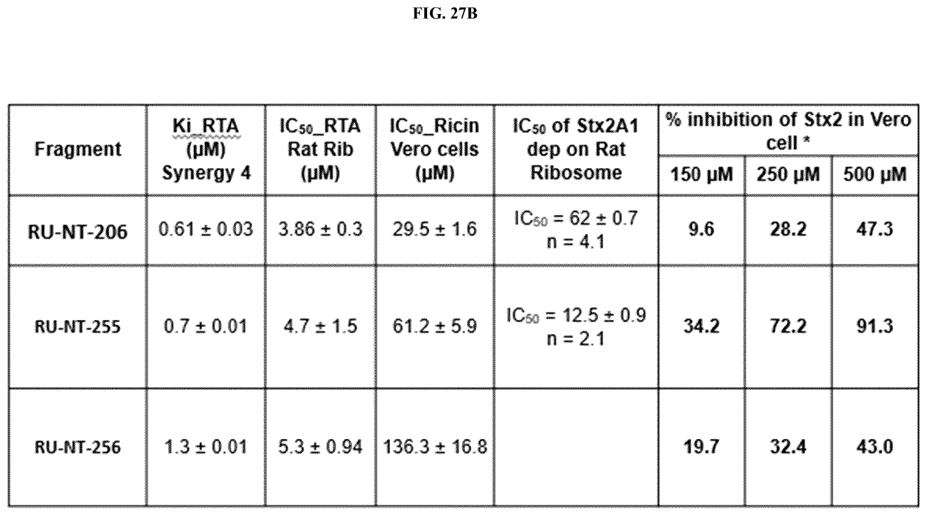

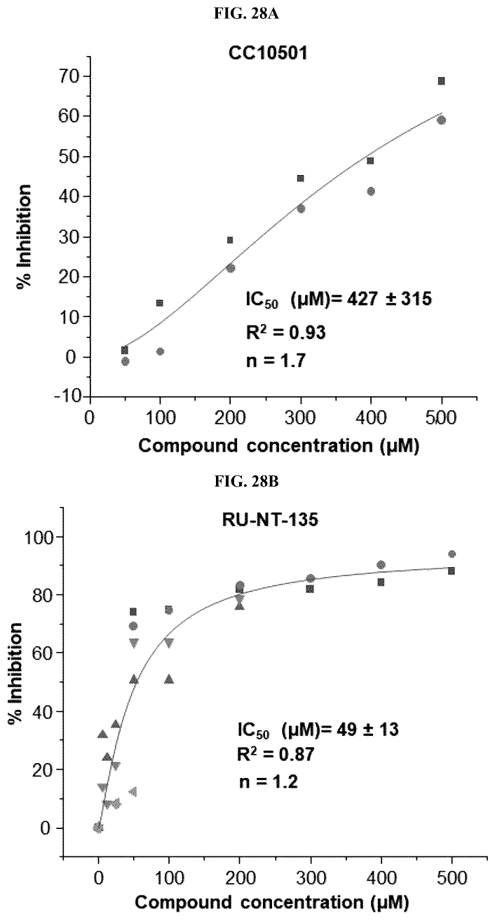

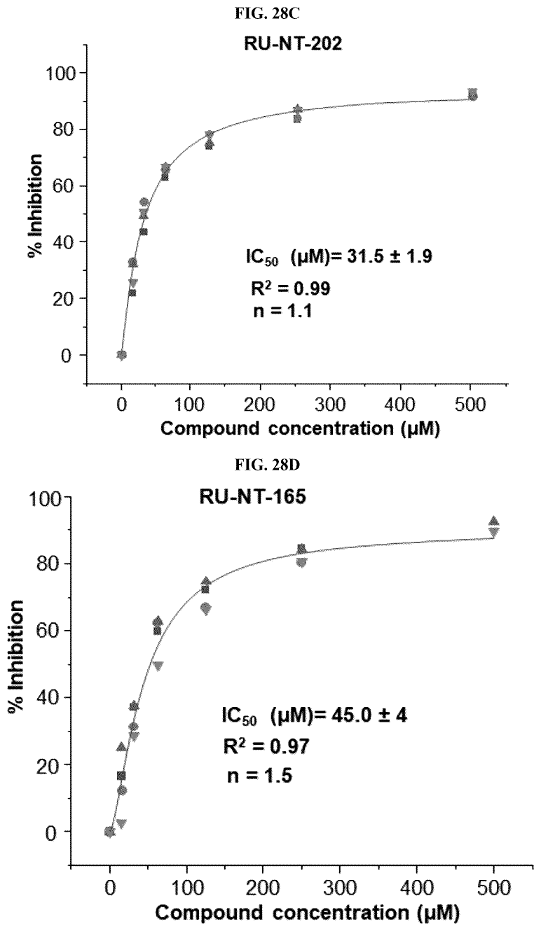

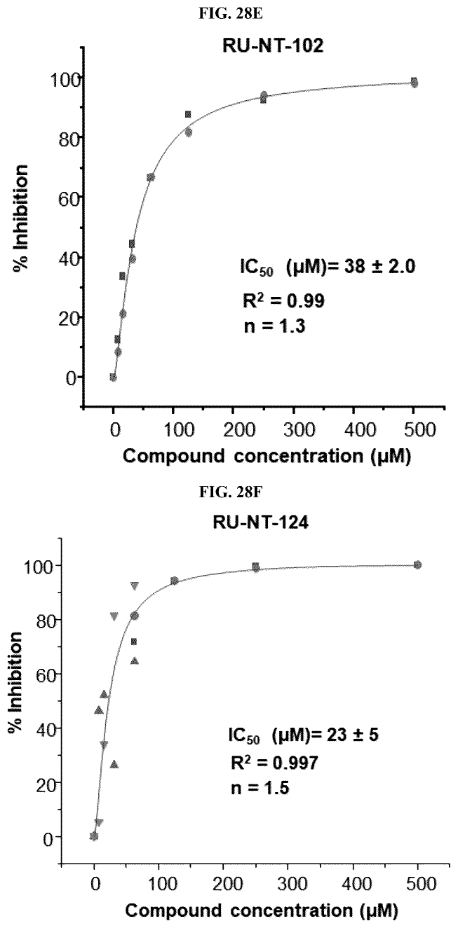

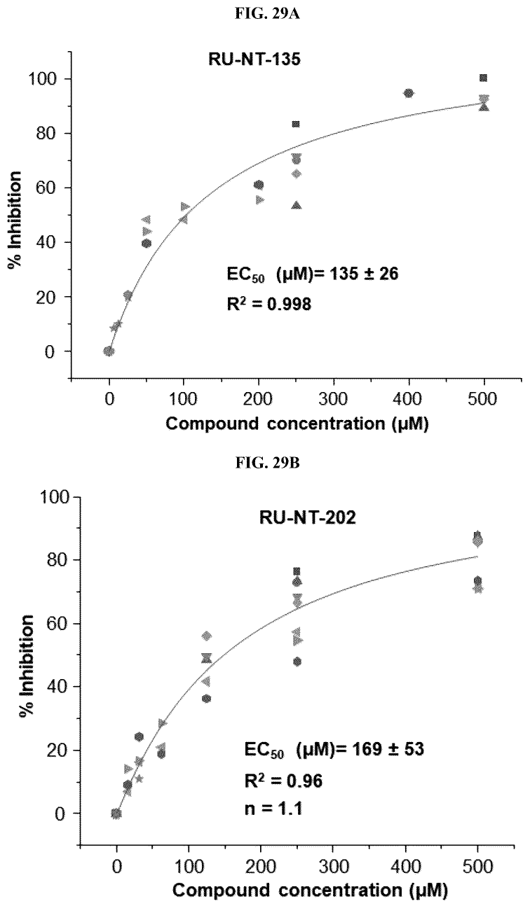

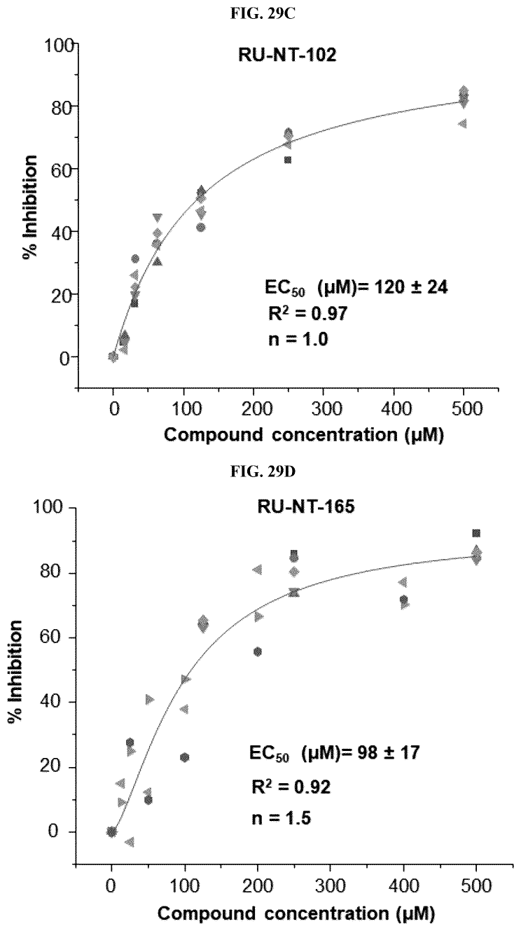

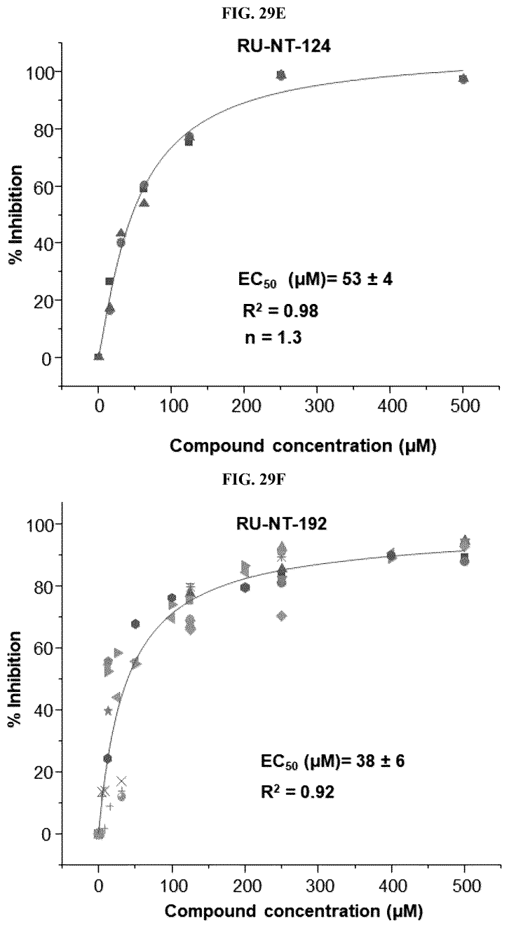

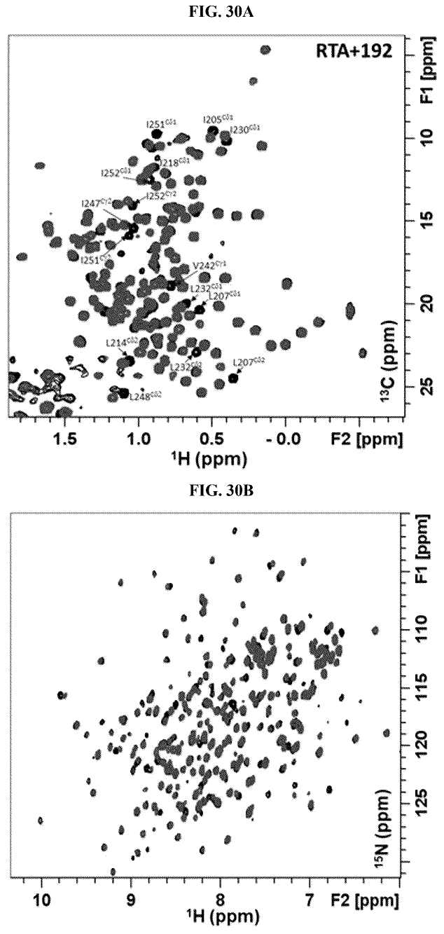

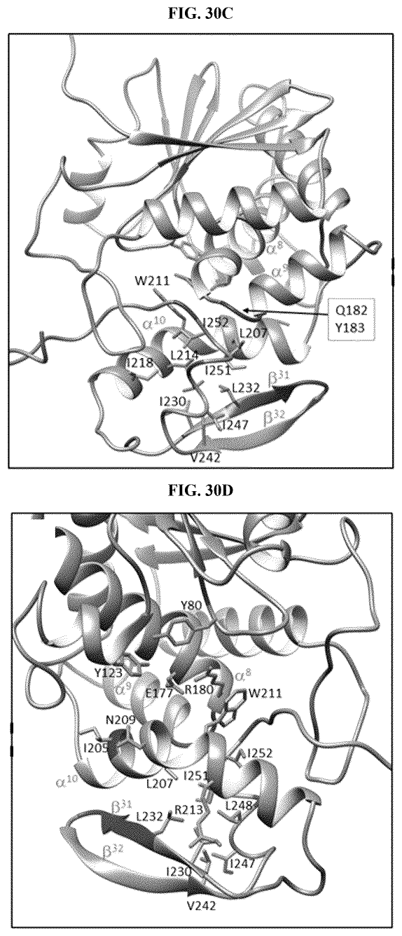

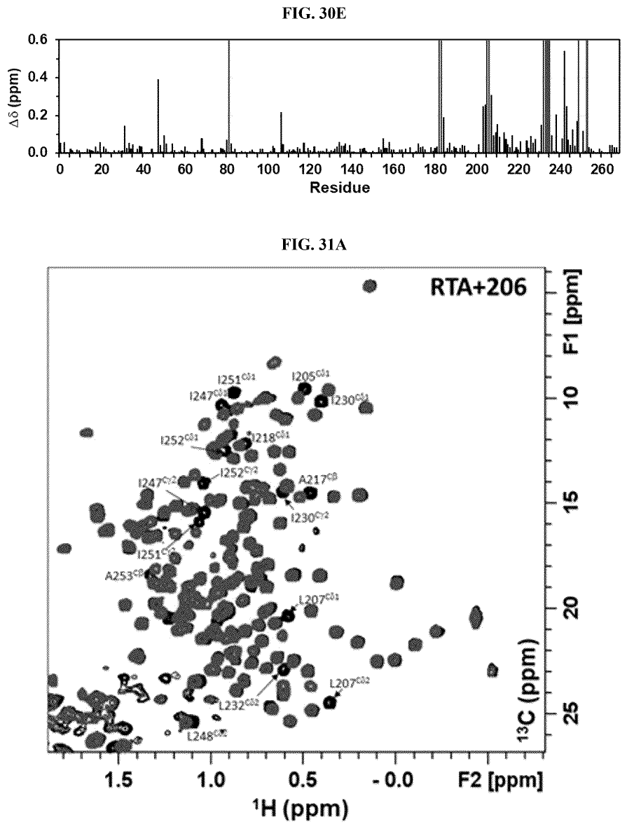

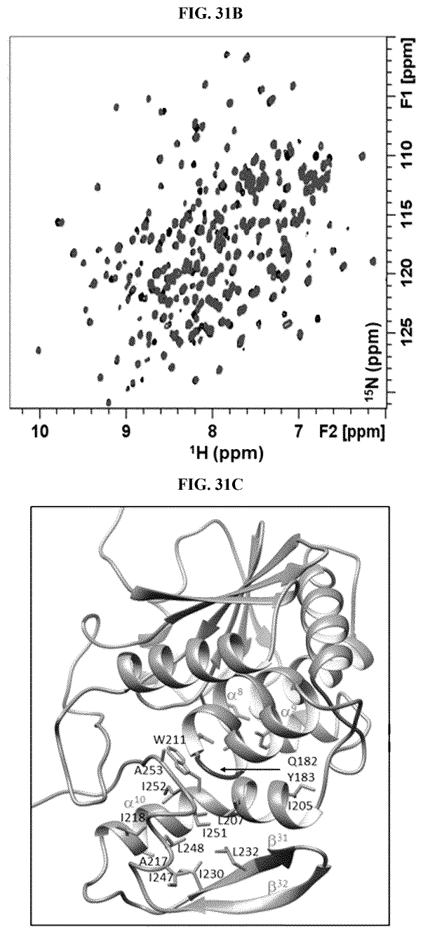

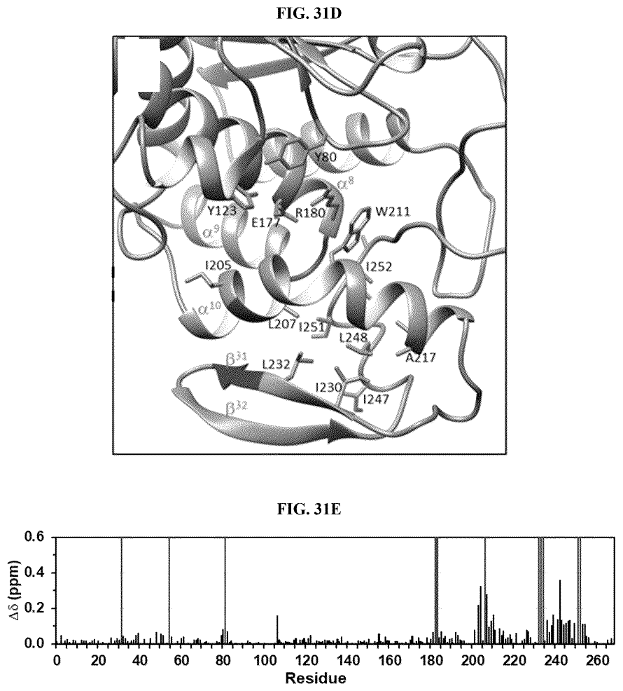

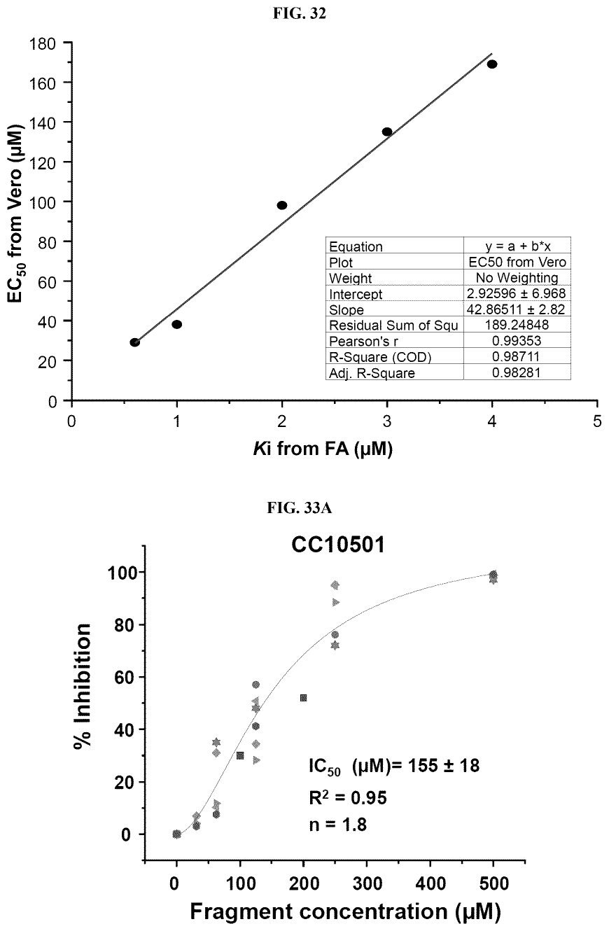

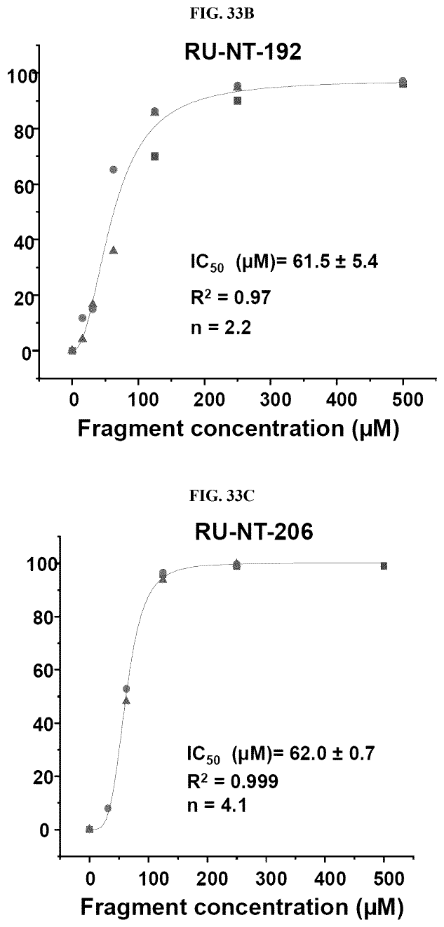

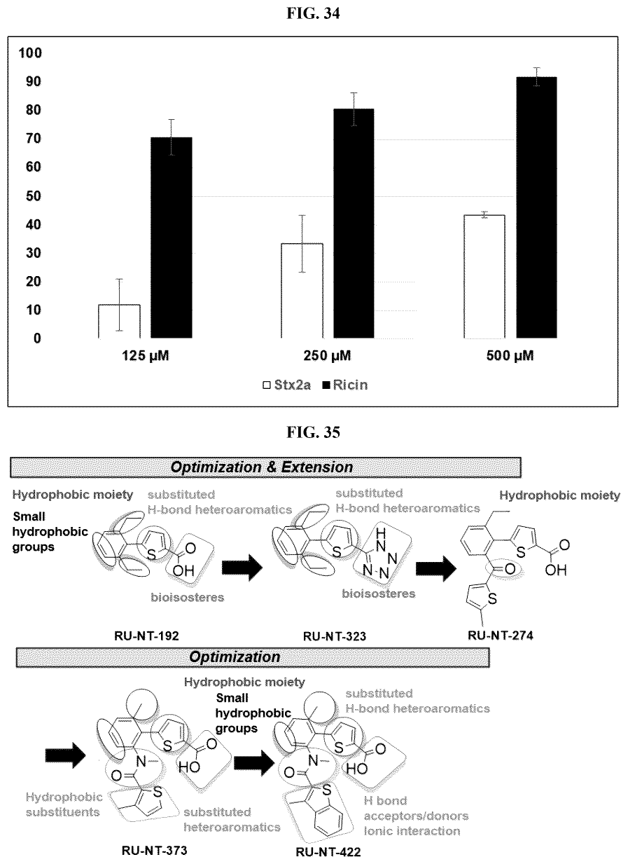

A fluorescence polarization (FP) competition assay was developed that detects competition of the fluorescently labeled P11 peptide with small molecule inhibitors to determine the binding affinity between the small molecule inhibitors and RTA. P11 was selected for the initial experiments based on the highest binding affinity (KD) for RTA. P11 was labeled with the BODIPY™ TMR-X NHS ester dye (ThermoFisher) at the N-terminus and separated from a free dye by semi-preparative HPLC (ACCQPrep, Gemini 30×150 mm C-18 column. 5 mL/min flow rate, eluent A: 0.1% TFA in water, eluent B 0.1% TFA in acetonitrile; gradient: B 5 to 100% in 40 min). The labeling of the peptide was confirmed by high resolution mass spectrometry (FIRMS) analysis using an Agilent 6546 qTOF LC/MS system. The IRMS analysis of the unlabeled and BODIPY TMR-X labeled P11 are shown in FIGS. 19-20, respectively. The HRMS value calculated for MNa+ BODIPY TMR-X labeled P11 was 1733.67927 m/z, found 1733.6787 m/z, error: 0.33 ppm (FIG. 20). To determine the optimal concentration of the fluorescent P11 for the FP assay, the fluorescence polarization of the labeled P11 was measured as a function of its concentration. To determine the optimal concentration of RTA, binding of the fluorescently labeled P11 to purified recombinant RTA was analyzed by keeping the peptide concentration fixed at 1 μM and varying the concentration of RTA for 30 min at room temperature. The fluorescence polarization values, and the anisotropy values were calculated using the parallel and perpendicular intensities of the emitted fluorescence as shown in equations (Eqs. 1-3):

$$FP = \frac{F ‖ - F ⊥}{F ‖ + F ⊥} \qquad ( Eq . 1 )$$

$$A = \frac{F ‖ - F ⊥}{F ‖ + 2 F ⊥} \qquad ( Eq . 2 )$$

$$A = \frac{2 FP}{3 - FP} \qquad ( Eq . 3 )$$

The FP values obtained using equation (Eq. 1) were multiplied by 1000 and expressed in millipolarization (mP) units. For quantitative analysis, anisotropy values were used because they are additive in nature and the linear superposition principle is not valid for FP values. In a mixture of more than two interacting species, receptor protein RTA and the free and bound labeled P11 peptide, both in case of direct binding as well as competition experiments, the fraction bound (F_{b}) is related to the observed anisotropy values (A_{obs}) at a given concentration of RTA as shown in equation (Eq. 4):

$$Fb = \frac{Aobs - Afree}{( Abound - Aobs ) Q + ( Aobs - Afree )} \qquad ( Eq . 4 )$$

A_{free} and A_{bound} denote anisotropy values corresponding to the free and bound states of the labeled P11 peptide. The quantum yield of the fluorophore (Q) is calculated as the ratio of the total fluorescence intensity (F1+2F ⊥) of the bound and the free states of the labeled P11 peptide. The Fb data obtained is plotted against varying concentrations of RTA and the non-linear quadratic equation (Eq. 5) is used to derive the binding constant (KD) using OriginPro (OriginLab Corporations, USA).

$$Fb = \frac{( KD + Lt + Rt ) - \sqrt{\{ {( KD + Lt + Rt )}^{2} - 4 LtRt} \}}{2 Lt} \qquad ( Eq . 5 )$$

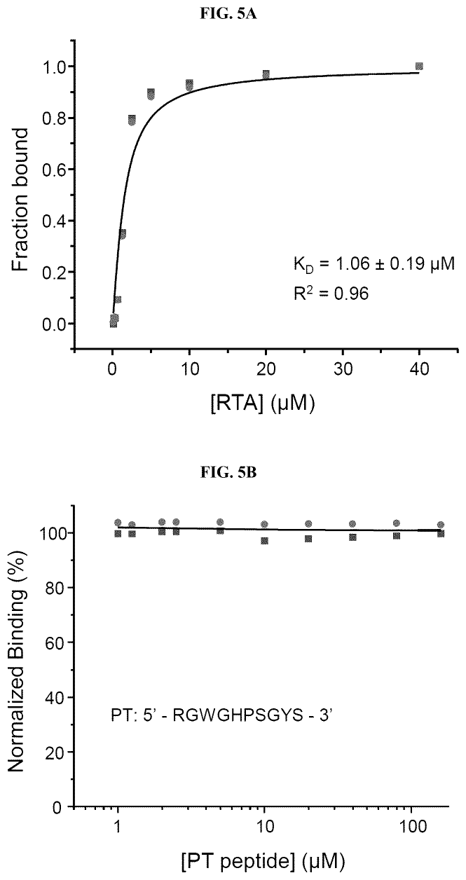

Lt and Rt are the total concentration of the ligand (labeled P11 peptide) and the concentration of RTA protein. In these experiments, Lt was kept constant at 1 μM and Rt varied from 0 to 40 μM. The measured KD value for binding between RTA and labeled P11 peptide is 1±0.2 μM (FIGS. 5A-5C).

### Measurement of Kᵢ Values by the FP Assay

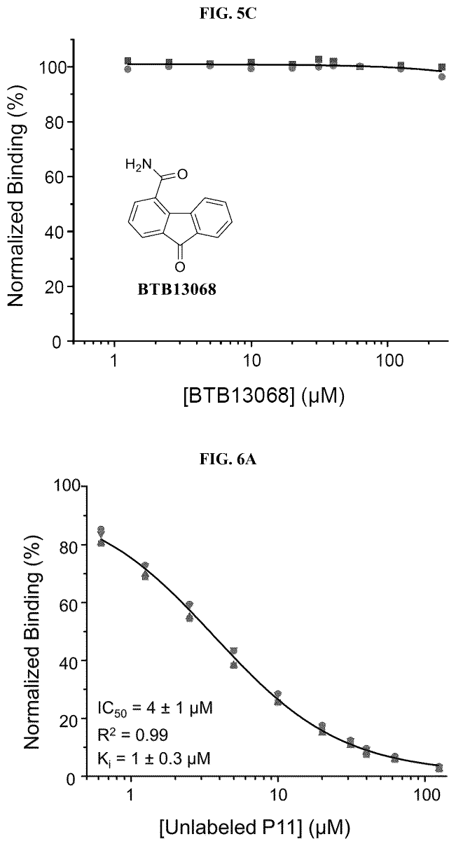

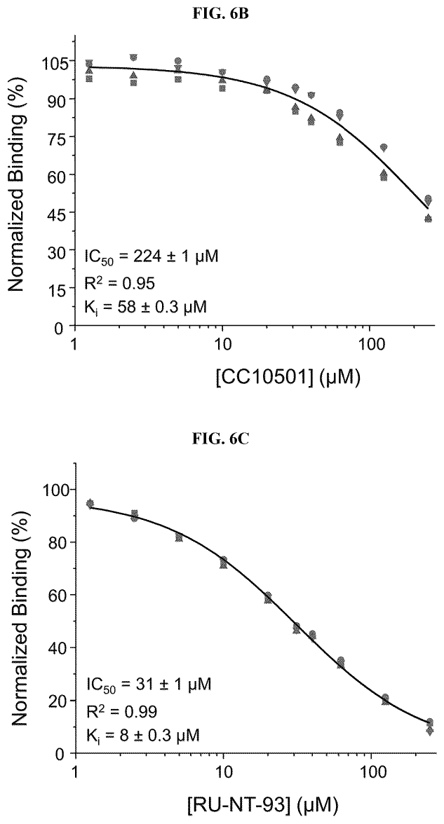

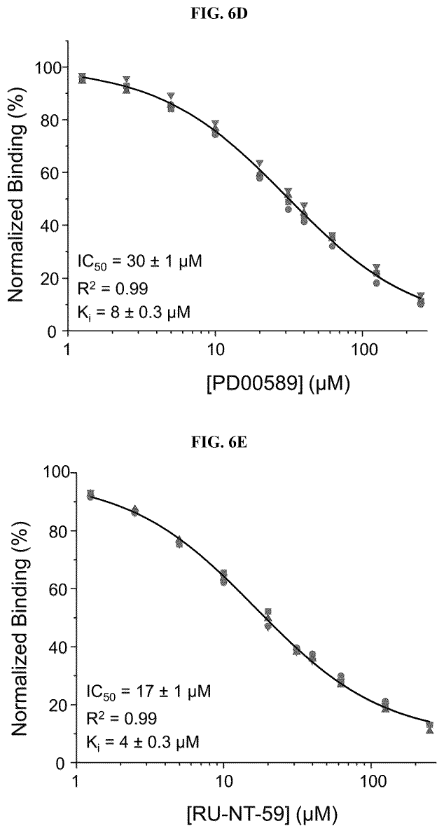

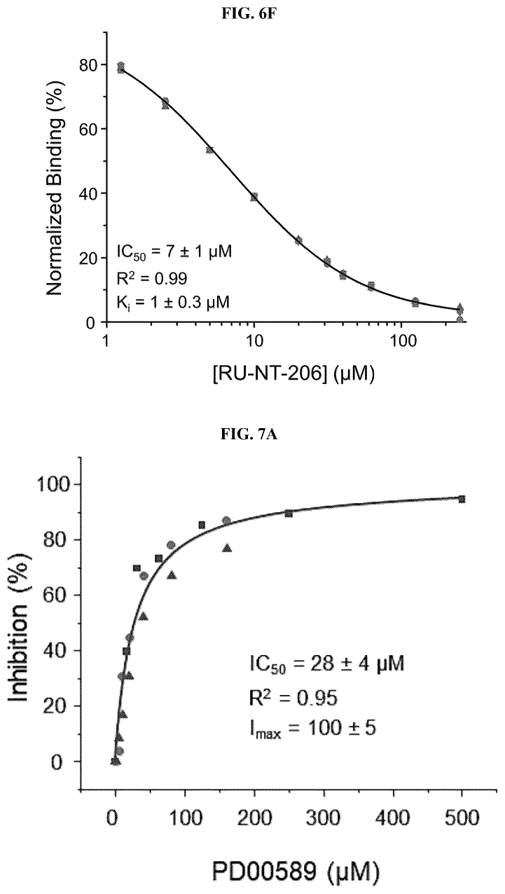

To determine if small molecules can displace the fluorescently labeled P11 peptide, varying concentrations of small molecule inhibitors (5, 10, 20, 40, 80, 125, 160, 250, 500, and 1000 μM) were incubated with 3 μM of RTA and 1 μM labeled P11 in reaction buffer containing 25 mM Tris-Cl pH 8.0, 100 mM NaCl and 1% DMSO in a black 96-well plate (Coming #3993). The reaction volume was made up to 40 μL along with two control sets: one with 3 μM of RTA and 1 μM of labeled P11 corresponding to the bound state dataset and another with only 1 μM of labeled P11 corresponding to the free state observables. The microplate was centrifuged at 400×g for 3 min and was incubated in the dark for 30 min at room temperature followed by centrifugation again before scanning. After half an hour, the samples were scanned using a BioTek Synergy 4 microplate reader with an Excitation filter of 530/25 nm and an Emission filter of 590/35 nm. The experiments were repeated four times as shown in FIGS. 6A-6F. The dynamic range of the assay, i.e., the difference between the Polarization values between labeled P11 peptide alone and labeled P11 peptide bound with RTA is >100 millipolarization (mP) units for all the subsequent experiments. The normalized inhibition (%) was calculated using equation (Eq. 6) where A_{obs} are the anisotropy values obtained by varying the concentration of the competitor, Abound are the anisotropy values corresponding to the bound state dataset having RTA and labeled P11 incubated together and A^{free} are the anisotropy values corresponding to the free state dataset having labeled P11 only.

$$NormalizedBinding ( \% ) = \frac{Abound - Aobs}{( Abound - Aobs ) Q + ( Aobs - Afree )} \times 100 \qquad ( Eq . 6 )$$

$$Aobs = Afree + \frac{Abound - Afree}{1 + {10}^{( Logx - LogIC 50 )}} \qquad ( Eq . 7 )$$

$$IC 50 = ( \frac{F 0 \times KD}{( 1 - F 0 ) ( 2 - F 0 )} + \frac{F 0 \times Lt}{2} ) ( \frac{Ki ( 2 - F 0 )}{Kd \times F 0} + 1 ) \qquad ( Eq . 8 )$$

$$Ki = [ \frac{IC 50}{\{ \frac{F 0 KD}{( 1 - F 0 ) ( 2 - F 0 )} \} + \{ \frac{F 0 Lt}{2} \}} - 1 ] \{ \frac{KD \cdot F 0}{2 - F 0} \} \qquad ( Eq . 9 )$$

For competition-based FP assays, RTA concentration must be chosen such that the fraction of labeled P11 bound over total (F0) is between 0.5 and 0.8.50 In these experiments, RTA concentration (Rt) is kept constant at 3 μM, labeled P11 (Lt) is 1 μM and KD value is 1 μM. Substituting these values in equation (Eq. 5), the F0 value is 0.7, which is in accordance with these criteria. The inhibitory constant, Kᵢ describes the binding affinity between the inhibitor and RTA and is defined as the concentration of the inhibitor that will bind to half of the binding sites on RTA at equilibrium in the absence of labeled P11. The Kᵢ value for each compound was calculated by solving equation (Eq. 8) for Kᵢ using equation (Eq. 9) where F0 is 0.7, K_{D} is 1 μM, Lt is 1 μM and the IC₅₀ values are obtained from equation (Eq. 7).

### Depurination Assay in Mammalian Cells

Both Vero and A549 cells were used in this study. The cells were maintained in Dulbecco's modified Eagle medium (DMEM) with penicillin, streptomycin, and 10% fetal calf serum supplements and were incubated at 37° C., 5% CO₂. Cells were prepared in the medium at 1.5×10⁵/mL in 24-well tissue culture plates at 500 μL per well and grown for 24 h. DMEM with penicillin and streptomycin minus serum was prepared with each toxin in volume adequate for the experiment. Ricin holotoxin was added to a final concentration of 200 pM and Stx2a was added to a final concentration of 2 nM. Aliquots of the medium containing the toxin were distributed to Eppendorf tubes and compounds in 100% DMSO stocks were added at the designated final compound concentrations for each treatment and vortexed to dissolve well. The final concentration of DMSO in the controls and the treatments was 0.5%. The medium containing serum was removed from each well of the 24-well culture plate and replaced with 400 L of the compound/toxin medium lacking serum. The plate was incubated for 2 h prior to harvest. The medium was then removed, and cells were collected in 350 μL of the lysis buffer from the Qiagen RNeasy® Plus Mini kit (Qiagen). Total RNA was extracted from the cells using the Qiagen kit either immediately or after storage at −80° C. The High-Capacity cDNA Reverse Transcription Kit (Thermo Fisher Scientific) was used for cDNA conversion of ~375 ng of total RNA in a 20 μL reaction.

Depurination Inhibition by qRT-PCR

All qRT-PCR assays were performed with a StepOnePlus Real Time PCR System (Applied Biosystems). Optimized reactions were done in triplicate in a total volume of 20 L using 5 μL of cDNA diluted 50-fold from the RT reaction using Power SYBR Green Master (Applied Biosystems). Forward and reverse primers used were as follows: 28S rRNA, 5′-GATGTCGGCTCTTCCTATCATTGT-3′ (SEQ ID NO:2) and 5′-CCAGCTCACGTTCCCTATTAGTC-3′ (SEQ ID NO:3); Depurinated rRNA, 5′-TGCCATGGTAATCCTGCTCAGTA-3′ (SEQ ID NO:4) and 5′-TCTGAACCTGCGGTTCCACA-3′ (SEQ ID NO:5). Primer concentrations in the final reaction volume were all at 250 nM. The comparative CT method (88CT) was used for quantification where depurinated rRNA is normalized to a total of 28S rRNA. Normalized amounts of depurinated rRNA from the experimental samples were then normalized to control samples. Data from a minimum of two biological replicates was used for analysis.

### Cloning and Protein Production for Crystallization

The PCR amplicon for RTA residues 1-267 was subcloned into the pSUMO expression vector encoding an N-terminal deca-histidine and SUMO tag. All cloning was performed using a standard ligase-independent cloning protocol. RTA was expressed in E. coli strain BL21(DE3). The transformed bacteria were grown at 37° C. in TB medium and induced at 20° C. with 0.1 mM IPTG at an Od₆00 of 0.6 for ~16 h. After induction, cells were harvested and resuspended in 20 mM Tris-Cl pH 7.5 and 150 mM NaCl. The cell suspension was sonicated and centrifuged at 30,000×g for 30 min. After centrifugation, the protein-containing supernatant was purified by nickel-affinity and size-exclusion chromatography on an AKTAxpress system (GE Healthcare), which consisted of a 1 mL nickel affinity column followed by a Superdex 200 16/60 gel filtration column. The elution buffer consisted of 0.5 M imidazole in the binding buffer, and the gel filtration buffer consisted of 20 mM HEPES pH 7.6, 150 mM NaCl, and 20 mM imidazole. Fractions containing RTA were pooled and subject to TEV protease cleavage (1:10 wt ratio) for 3 h at room temperature to remove the sumo fusion tag. The cleaved RTA was passed over a 1 mL Ni-NTA agarose (Qiagen) gravity column to remove TEV protease, cleaved residues, and uncleaved fusion protein. RTA was buffer exchanged into 20 mM Hepes pH 7.5, 150 mM NaCl, and 1 mM TCEP before complexation with each fragment inhibitor.

### Crystallization and Data Collection

To generate each RTA-inhibitor complex for crystallization trials, RTA was concentrated to 10 mg/ml incubated with 2 mM of each inhibitor for 30 min at room temperature, and then put into crystallization trials. All RTA-inhibitor crystals were grown by sitting drop vapor diffusion at 20° C. using a protein-to-reservoir volume ratio of 1:1 with total drop volumes of 0.2 μL. All crystals were flash-frozen in liquid nitrogen after a short soak in the appropriate crystallization buffers supplemented with 20-25% ethylene glycol. Data were collected at the 24-ID-E beamline at the Advanced Photon Light Source (APS), Argonne National Labs. All data was indexed, merged, and scaled using HKL2000 then converted to structure factors using CCP4 7.0.

### Structure Determination and Refinement

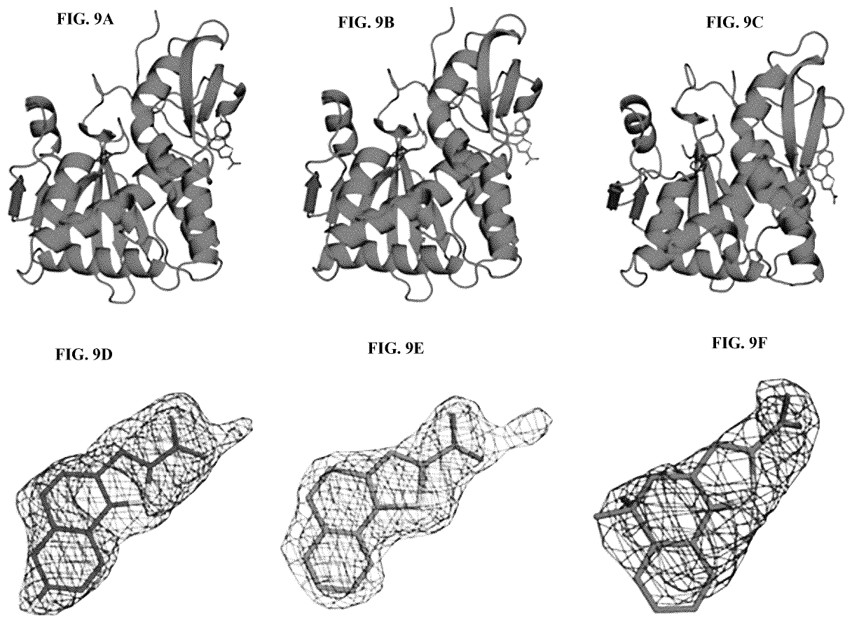

Each RTA-inhibitor complex was solved by molecular replacement. Molecular replacement calculations were performed using the RTA coordinates (PDB ID: 1RTC) as a search model for all RTA-inhibitor complexes. The resulting phase information from molecular replacement was used to identify and place each fragment inhibitor into the resulting electron density maps using the molecular graphics program COOT 8.9.2. The electron density corresponding to the ligands bound to RTA was well defined in the original difference density maps (FIGS. 9D-9F). The structures of the RTA-RU-NT-59, RTA-PD00589, and RTA-RU-NT-206 complexes were solved at 1.9 Å, 2.3 Å, and 2.7 Aresolution, respectively, in the P6322 space group. Each RTA-inhibitor complex had one copy of RTA in the asymmetric unit. Structural refinement of all coordinates was performed using the PHENIX 1.20.1 package. During refinement, a cross-validation test set was created from a random 5% of 16 the reflections. Molecular graphics were prepared using PyMOL 4.6 (Schrodinger) (DeLano Scientific LLC, Palo Alto, CA). Each fragment inhibitor was left out of the model in the initial stages of refinement. After a few cycles of refinement, each fragment inhibitor was fitted into their respective electron densities and refined to convergence. B-factor analysis was done using the BAVERAGE program of the CCP4 7.0 suite. Some of the structural analysis was performed using the virtual reality software, Nanome.

### Statistical Analysis

Statistical analysis (ANOVA and mean comparisons) was performed with R (version 4.3.0, R Core Team, 2023) using RStudio (version 2023.3.1.446, Posit Team, 2023). The Dunnett's Test using the DescTools package (version 0.99.50) was used to compare treatment (ricin or Stx2 treated cells with compound) means to the control (ricin treated cells without compound). NS p>0.05, * p<0.05, * p<0.01, * * * p<0.001.

### In Vitro Depurination Inhibition

In vitro depuration inhibition assay was carried out as published. In brief, RTA was mixed with different concentrations of compounds in the depurination buffer (20 mM Hepes pH 7.5, 25 mM KCl and 5 mM MgCl₂), and rat liver ribosomes were added to start the reaction. The reaction was set at room temperature for 5 minutes, which is in the linear range of the depurination reaction. The reaction was stopped by adding an equal amount of 2X RNA extraction buffer (50 mM Tris-HCl pH 8.8, 240 mM NaCl, 20 mM EDTA and 2% SDS), and RNA was extracted and the depurination level was measured by qRT-PCR. A reaction without RTA and compound was set as no depurination control and a reaction with RTA but no compound was set as 100% depurination control for each experiment. The experiment was repeated 2-4 times.

### Mammalian Cell Viability Assay

Cell viability was assessed using Cell Titer Glo-3D reagent (Promega, #G9682). For compound screening, 100 μL of mammalian cells (1.5×10⁵ cells/mL) were added to each well of a sterile, white tissue culture-treated 96-well plate (Corning #3917) and allowed to grow for 24 h. After 24 h, the media was removed from the wells and replaced with 100 μL of media containing varying concentrations of compound and ricin. For compound preparation, compounds were diluted in DMEM media minus serum (Gibco, #11960044) and DMSO (Sigma, #D2650) to the appropriate concentrations. The final concentration of DMSO was 0.1% in all wells. Compounds were screened at 500 μM, 250 μM, 125 μM, and 0 pM final concentrations. Ricin (Vector Labs) was diluted in DMEM media minus serum to a final concentration of 20,000 pM and serially diluted 1:3 to generate 8 concentrations ranging from 0 to 20,000 pM final concentration. 55 μL of each compound concentration was mixed with 55 μL of each ricin concentration in a separate, sterile, non-tissue culture-treated 96-well plate (Corning, #351172). 100 μL of the compound/ricin mixture was then added to the corresponding wells of the 96-well plate containing the mammalian cells. The plates were incubated for 24 h at 37° C. After 24 h, the plates were removed from the incubator and allowed to equilibrate to room temperature for 15 min. Cell Titer Glo-3D reagent (100 μL) was added to each well, mixed by pipetting up and down several times and the plates were shaken for 5 min. The plates were then incubated at room temperature for 15 min protected from light. Luminescence was measured using a BioTek plate reader. Data were analyzed using OriginPro 2023 software.

### Example 1: Synthesis of Certain Exemplary Compounds of the Disclosure

### 1-Chloro-6-fluoro-3,4-dihydronaphthalene-2-carbaldehyde (2a)

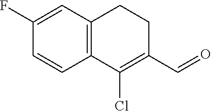 [SMILES: O=CC1=C(Cl)c2ccc(F)cc2CC1]

DMF (0.62 mL, 8 mmol, 8 equiv.) was slowly added to a well-stirred cooled solution of POCl₃ (0.66 mL, 7 mmol, 7 equiv.) at 0° C., as soon as the reaction mixture precipitated, 6-fluoro-3,4-dihydronaphthalen-1 (2H)-one (164 mg, 1 mmol) was diluted in a small amount of DMF and was added to the reaction mixture. Afterward, the reaction mixture was warmed to room temperature and then heated to 70° C. for 2 h. After cooling to room temperature, the mixture was diluted with ethyl acetate and poured into crushed ice. The aqueous layer was extracted with ethyl acetate. The combined organic layers were successively washed with sodium bicarbonate, and brine and dried over sodium sulfate, filtered, and concentrated. The residue was used in the next step without further purification. ESI-MS: 211.0 [M+H]+.

### 6-Bromo-1-chloro-3,4-dihydronaphthalene-2-carbaldehyde (2b)

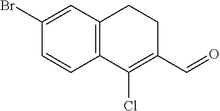 [SMILES: O=CC1=C(Cl)c2ccc(Br)cc2CC1]

The compound has been prepared according to the procedure for 2a. ESI-MS: 270.8 (M+H)+. 5.1.3. Methyl 7-fluoro-4,5-dihydronaphtho[1,2-b]thiophene-2-carboxylate (3a) To a stirred solution of 1-chloro-6-fluoro-3,4-dihydronaphthalene-2-carbaldehyde (210 mg, 1 mmol, 1 equiv.) in pyridine (2 mL) methyl 2-mercaptoacetate (0.103 mL, 1.15 mmol, 1.15 equiv.) and triethylamine (0.223 mL, 1.6 mmol, 1.6 equiv.) were added. The mixture was heated at 60° C. for 2 h and then left to cool down to RT. An aqueous solution of 50% (w/w) KOH (0.165 mL) was added and the mixture was stirred for another 20 min. The medium was poured over ice and rinsed with dichloromethane before adding dropwise a solution of HCl (0.5 mL, 1 M). The organic layer was extracted and washed with 1 M HCl and water, dried over anhydrous Na₂SO₄, filtered, and concentrated. The residue was purified by flash chromatography to give the product (189 mg, 72%, yellow solid). ESI-MS: 263.0 (M+H)+.

### Methyl 7-bromo-4,5-dihydronaphtho[1,2-b]thiophene-2-carboxylate (3b)

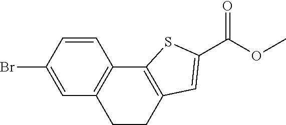 [SMILES: COC(=O)c1cc2c(s1)-c1ccc(Br)cc1CC2]

The compound has been prepared according to the procedure for 3a. ESI-MS: 322.8 (M+H)+.

### Methyl 7-methyl-4, 5-dihydronaphtho[1,2-b]thiophene-2-carboxylate (3c)

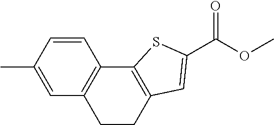

A mixture of methyl 7-bromo-4,5-dihydronaphtho[1,2-b]thiophene-2-carboxylate (3b, 65 mg, 0.2 mmol), methylboronic acid (36 mg, 0.6 mmol, 3 equiv.), palladium acetate (2 mg, 0.008 mmol, 0.04 equiv.), tricyclohexylphosphine (PCy3, 5 mg, 0.018 mmol, 0.09 equiv.), and K3PO4 (142 mg, 0.664 mmol, 3.3 equiv.) was dissolved in toluene (10 mL) and water (1 mL). The mixture was flushed with nitrogen for 3 min and then refluxed under nitrogen overnight. After cooling to room temperature, the reaction mixture was partitioned between water and ethyl acetate. The organic layer was washed with water and brine and dried over anhydrous Na₂SO₄, filtered, and concentrated. The residue was purified by flash chromatography (hexane:ethyl acetate 0%-15%) to give the corresponding ester (38 mg, 75% yield). ESI-MS: 258.9 (M+H)+.

### Methyl 7-(dimethylamino)-4,5-dihydronaphtho[1,2-b]thiophene-2-carboxylate (3d)

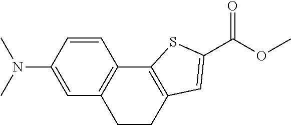

A mixture of methyl 7-bromo-4,5-dihydronaphtho[1,2-b]thiophene-2-carboxylate (3b, 44 mg, 0.136 mmol), dimethylamine (2.0 M in THF, 0.2 mL, 0.41 mmol, 3 equiv.), palladium acetate (3 mg, 0.0136 mmol, 0.1 equiv.), BINAP (8.5 mg, 0.0136 mmol, 0.1 equiv.), and cesium carbonate (134 mg, 0.41 mmol, 3 equiv.) were dissolved in dioxane (5 mL). The mixture was flushed with nitrogen for 3 min and then refluxed under nitrogen overnight. After cooling to room temperature, the reaction mixture was partitioned between water and ethyl acetate. The organic layer was washed with water and brine and dried over anhydrous Na₂SO₄, filtered, and concentrated. The residue was purified by flash chromatography (hexane:ethyl acetate 0%-15%) to give the corresponding ester (31 mg, 80% yield). ESI-MS: 287.9 (M+H)+.

### Methyl 7-methoxy-4, 5-dihydronaphtho[1,2-b]thiophene-2-carboxylate (3e)

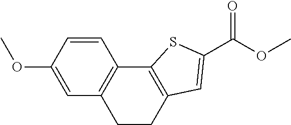

A mixture of methyl 7-bromo-4,5-dihydronaphtho[1,2-b]thiophene-2-carboxylate (3b, 50 mg, 0.155 mmol), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (BrettPhosPdG3, 3 mg, 0.0031 mmol, 0.02 equiv.), and cesium carbonate (71 mg, 0.21 mmol, 1.4 equiv.) was dissolved in dioxane (2 mL) and methanol (2 mL). The mixture was flushed with nitrogen for 5 min and then refluxed under nitrogen for an hour. After cooling to room temperature, the reaction mixture was concentrated, and the residue was partitioned between water and ethyl acetate. The organic layer was washed with water and brine and dried over anhydrous Na₂SO₄, filtered, and concentrated. The residue was purified by flash chromatography (hexane:ethyl acetate 0%-15%) to give the corresponding ester (17 mg, 40% yield). ESI-MS: 275.4 (M+H)+.

### 5-Phenylthiophene-2-carboxylic acid (CC10501)

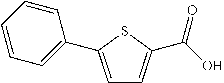 [SMILES: O=C(O)c1ccc(-c2ccccc2)s1]

The compound (white solid) was purchased from Maybridge (part of Thermo Scientific since 2021) and used as is. ¹H NMR: (500 MHz, DMSO-d₆) δ 7.77-7.68 (m, 3H), 7.58 (d, J=3.9 Hz, 1H), 7.50-7.43 (m, 2H), 7.43-7.38 (m, 1H). HRMS: C₁₁H₇O₂S, [M+H]+ calc. 203.01722, found 203.0167, err. δ 2.6 ppm.

### 4,5-Dihydronaphtho[1,2-b]thiophene-2-carboxylic acid (PD00589)

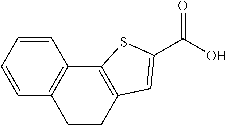 [SMILES: O=C(O)c1cc2c(s1)-c1ccccc1CC2]

The compound (white solid) was purchased from Maybridge and used as is. ¹H NMR: (300 MHz, DMSO-d₆) δ 13.04 (s, 1H), 7.59 (s, 1H), 7.46-7.39 (m, 1H), 7.33-7.23 (m, 3H), 2.92 (dd, J=9.4, 6.6 Hz, 2H), 2.84-2.75 (m, 2H). HRMS: [M+H]+ calc. 231.0480, found 231.0502.

### 4,5-Dihydronaphtho[1,2-b]thiophene-2-carbohydrazide (PD00633)

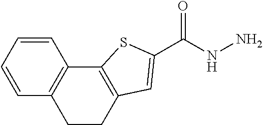 [SMILES: NNC(=O)c1cc2c(s1)-c1ccccc1CC2]

The compound was purchased from Maybridge as a 100 mM solution in DMSO (part of Thermo Scientific since 2021) and used as is.

### 8-Chloro-4H-thieno[3,2-c]thiochromene-2-carbohydrazide (SEW01689)

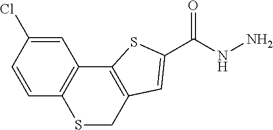 [SMILES: NNC(=O)c1cc2c(s1)-c1cc(Cl)ccc1SC2]

The compound was purchased from Maybridge as a 100 mM solution in DMSO and used as is.

### Methyl 8-fluoro-4H-thieno[3,2-c]chromene-2-carboxylate (SEW01765)

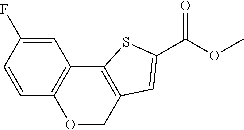 [SMILES: COC(=O)c1cc2c(s1)-c1cc(F)ccc1OC2]

The compound was purchased from Maybridge as a 100 mM solution in DMSO and used as is.

### 1-(8-Chloro-4H-thieno[3,2-c]thiochromen-2-yl)ethan-1-one (SEW02679)

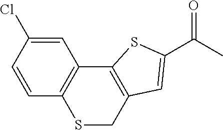 [SMILES: CC(=O)c1cc2c(s1)-c1cc(Cl)ccc1SC2]

The compound was purchased from Maybridge as a 100 mM solution in DMSO and used as is.

### 8-Chloro-4H-thieno[3,2-c]thiochromene-2-carboxylic acid (SEW01776)

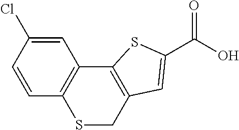 [SMILES: O=C(O)c1cc2c(s1)-c1cc(Cl)ccc1SC2]

The compound (bright yellow solid) was purchased from Maybridge and used as is. The purity of the sample was 85%. ¹H NMR: (500 MHz, DMSO-d₆) δ 7.61-7.56 (m, 2H), 7.43 (m, 1H), 7.31 (m, 1H), 4.07 (d, J=1.0 Hz, 2H). HRMS: C12H6ClO2S2+, [M+H]+ calc. 280.95032, found 280.9513, err. 3.5 ppm.

### 7-Fluoro-4,5-dihydronaphtho[1,2-b]thiophene-2-carboxylic acid (RU-NT-59)

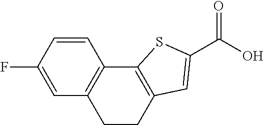 [SMILES: O=C(O)c1cc2c(s1)-c1ccc(F)cc1CC2]

To a stirred solution of methyl 7-fluoro-4,5-dihydronaphtho[1,2-b]thiophene-2-carboxylate (3a, 50 mg, 0.19 mmol, 1 equiv.) in MeOH (3 mL) and THF (3 mL) was added 1 M NaOH (4 mL). The mixture was stirred at room temperature for 2 h. After completion of the reaction by LC-MS, the reaction mixture was partitioned between 1 M HCl and ethyl acetate. The organic layer was washed with water, and brine and dried over anhydrous Na₂SO₄, filtered, and concentrated to afford the product as a pale-yellow solid (42 mg, 90%). Subsequently, the product was additionally purified by prep-HPLC (ACCQPrep, C18, liquid injection, (water/formic acid 0.1%)/CH₃CN, 10%-100%). Fractions, that contained the product (by LCMS), were combined to give the desired product as a white solid. ¹H NMR: (500 MHz, DMSO-d₆) δ 7.58 (s, 1H), 7.47 (dd, J=8.5, 5.6 Hz, 1H), 7.20 (dd, J=9.6, 2.7 Hz, 1H), 7.10 (td, J=8.7, 2.8 Hz, 1H), 2.93 (dd, J=8.8, 6.6 Hz, 2H), 2.79 (dd, J=8.9, 6.6 Hz, 2H). ¹³C NMR: (126 MHz, DMSO-d₆) δ 163.05, 162.76, 160.81, 140.43, 138.31, 138.25, 137.67, 133.47, 126.79, 125.25, 115.52, 113.88, 28.16, 22.75. ESI-MS: 247.2 [M+H]+. HRMS: C13H10FO2S+, [M+H]+ calc. 249.0386, found 249.0385.

### 7-Bromo-4,5-dihydronaphtho[1,2-b]thiophene-2-carboxylic acid (RU-NT-61)

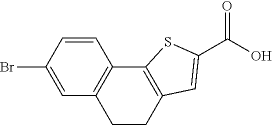 [SMILES: O=C(O)c1cc2c(s1)-c1ccc(Br)cc1CC2]

The compound (white solid) has been prepared from 3b according to the procedure for RU-NT-59. The purity of the sample was 92%. ¹H NMR: (500 MHz, DMSO-d₆) δ 7.61 (s, 1H), 7.56 (dd, J=2.1, 1.0 Hz, 1H), 7.46 (dd, J=8.2, 2.1 Hz, 1H), 7.39 (d, J=8.2 Hz, 1H), 2.93 (dd, J=8.9, 6.5 Hz, 2H), 2.80 (dd, J=8.9, 6.6 Hz, 2H). ¹³C NMR: (126 MHz, DMSO-d₆) δ 162.74, 138.43, 137.68, 133.53, 130.88, 129.85, 129.11, 124.98, 27.51, 22.62. HRMS: C13H879BrO2S+, [M+H]+ calc. 306.94339, found 306.9436, err. 1.7 ppm.

### 7-Methyl-4,5-dihydronaphtho[1,2-b]thiophene-2-carboxylic acid (RU-NT-62)

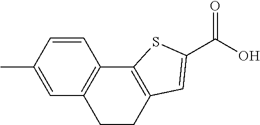 [SMILES: Cc1ccc2c(c1)CCc1cc(C(=O)O)sc1-2]

The compound (beige solid) has been prepared from 3c according to the procedure for RU-NT-59. ¹H NMR: (500 MHz, DMSO-d₆) δ ¹H NMR (500 MHz, DMSO-d₆) δ 8.16 (s, 1H), 7.56 (s, 1H), 7.32 (d, J=7.7 Hz, 1H), 7.14-7.05 (m, 2H), 2.87 (dd, J=8.8, 6.4 Hz, 2H), 2.81-2.74 (m, 2H), 2.29 (s, 3H). ¹³C NMR: (101 MHz, DMSO-d₆) δ 163.06, 137.84, 137.35, 135.13, 133.35, 128.99, 127.70, 127.44, 123.17, 28.09, 23.10, 20.90. HRMS: C14H11O2S+, [M+H]+ calc. 243.04852, found 243.0494, err. 3.6 ppm.

### 7-(Dimethylamino)-4,5-dihydronaphtho[1,2-b]thiophene-2-carboxylic acid (RU-NT-63)

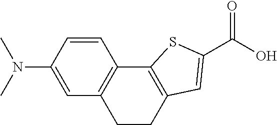 [SMILES: CN(C)c1ccc2c(c1)CCc1cc(C(=O)O)sc1-2]

The compound (neon green solid) has been prepared from 3d according to the procedure for RU-NT-59. The purity of the sample was 92%. ¹H NMR: (500 MHz, DMSO-d₆) δ 8.27 (s, 1H), 7.33 (s, 1H), 7.20 (d, J=8.3 Hz, 1H), 6.64 (s, 1H), 6.59 (d, J=8.4 Hz, 1H), 2.93 (d, J=2.3 Hz, 6H), 2.83 (d, J=7.7 Hz, 2H), 2.71 (d, J=7.7 Hz, 2H). HRMS: C15H15NO2S+, [M]+ calc. 273.08180, found 273.0816, err. 0.7 ppm.

### 7-Methoxy-4,5-dihydronaphtho[1,2-b]thiophene-2-carboxylic acid (RU-NT-64)

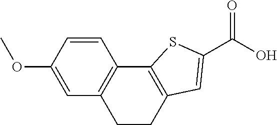 [SMILES: COc1ccc2c(c1)CCc1cc(C(=O)O)sc1-2]

The compound (yellow solid) has been prepared from 3e according to the procedure for RU-NT 59. The purity of the sample was 90%. ¹H NMR: (400 MHz, DMSO-d₆) δ 8.16 (s, 1H), 7.55 (s, 1H), 7.45-7.33 (m, 1H), 6.91 (d, J=2.6 Hz, 1H), 6.83 (dd, J=8.4, 2.6 Hz, 1H), 3.78 (s, 3H), 2.89 (dd, J=9.0, 6.4 Hz, 3H), 2.84-2.72 (m, 2H). ¹³C NMR: (101 MHz, DMSO-d₆) δ 163.11, 159.41, 141.83, 137.19, 136.25, 133.51, 124.64, 114.00, 112.56, 55.22, 28.47, 22.97. HRMS: C14H11O3S+, [M+H]+ calc. 259.04344, found 259.0439, err. 1.8 ppm.

### 5-(o-Tolyl)thiophene-2-carboxylic acid (RU-NT-70)

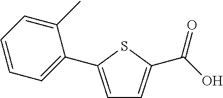

The synthesis and analysis of this compound have been previously reported in the literature.

### 5-Mesitylthiophene-2-carboxylic acid (RU-NT-75)

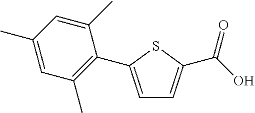 [SMILES: Cc1cc(C)c(-c2ccc(C(=O)O)s2)c(C)c1]

The synthesis and analysis of this compound have been previously reported in the literature.

### 5-(2,6-Dimethylphenyl)thiophene-2-carboxylic acid (RU-NT-93)

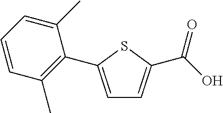 [SMILES: Cc1cccc(C)c1-c1ccc(C(=O)O)s1]

The synthesis and analysis of this compound have been previously reported in the literature.

### 4H-Thieno[3,2-c]chromene-2-carboxylic acid (RU-NT-198)

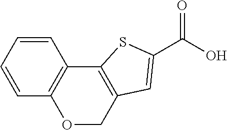 [SMILES: O=C(O)c1cc2c(s1)-c1ccccc1OC2]

The compound (white solid) was purchased from Enamine Ltd and used as is. ¹H NMR: (500 MHz, DMSO-d₆) δ 7.60 (s, 1H), 7.26 (ddd, J=8.2, 7.4, 1.6 Hz, 1H), 7.05-6.94 (m, 2H), 5.28 (s, 2H). HRMS: C12H7O3S+, [M+H]+ calc. 231.01214, found 231.0110, err. 4.9 ppm.

### 8-Fluoro-4H-thieno[3,2-c]thiochromene-2-carboxylic acid (RUNT-199)

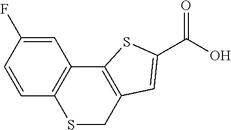 [SMILES: O=C(O)c1cc2c(s1)-c1cc(F)ccc1SC2]

The compound (beige solid) was purchased from Enamine Ltd and used as is. ¹H NMR: (500 MHz, DMSO-d₆) δ 13.32 (br s, 1H), 7.66 (s, 1H), 7.46 (d, J=8.4 Hz, 2H), 7.18-7.11 (m, 1H), 4.06 (s, 2H). ¹³C NMR: (101 MHz, DMSO-d₆) δ 162.54, 161.82, 159.40, 140.53, 135.51, 133.70, 133.28, 133.10, 132.82, 130.80, 129.94, 129.42, 126.99, 124.98, 116.05, 111.82, 67.57, 24.98. HRMS: C12H6FO2S2+, [M+H]+ calc. 264.97987, found 264.9797, err. 0.6 ppm.

### Benzo[b]thieno[2,3-d]thiophene-2-carboxylic acid (RU-NT-201)

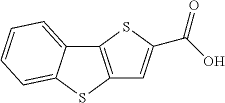 [SMILES: O=C(O)c1cc2sc3ccccc3c2s1]

The compound was purchased from Enamine Ltd. Subsequently, the compound was additionally purified by prep-HPLC to achieve the desired purity (ACCQPrep, C18, liquid injection, (water/formic acid 0.1%)/CH₃CN, 10%-100%). Fractions, that contained the product (by LCMS), were combined to give the desired product as a yellow solid. ¹H NMR: (500 MHz, DMSO-d₆) δ 8.17 (s, 1H), 8.14-8.04 (m, 2H), 7.54-7.45 (m, 2H). HRMS: C11H5O2S2+, [M+H]+ calc. 232.97364, found 232.9732, err. 1.9 ppm.

### 5,5-Dimethyl-4, 5-dihydronaphtho[1,2-b]thiophene-2-carboxylic acid (RU-NT-206)

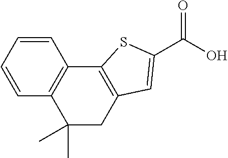 [SMILES: CC1(C)Cc2cc(C(=O)O)sc2-c2ccccc21]

The compound was purchased from Enamine Ltd. Subsequently, the compound was additionally purified by prep-HPLC to achieve the desired purity (ACCQPrep, C18, liquid injection, (water/formic acid 0.1%/CH₃CN), 10%-100%). Fractions, that contained the product (by LCMS), were combined to give the desired product as a white solid. ¹H NMR: (500 MHz, DMSO-d₆) δ 7.60 (s, 1H), 7.45 (ddd, J=7.6, 2.8, 1.4 Hz, 2H), 7.33 (td, J=7.5, 1.4 Hz, 1H), 7.27 (td, J=7.5, 1.4 Hz, 1H), 2.74 (s, 2H), 1.24 (s, 6H). ¹³C NMR: (101 MHz, DMSO) δ 162.97, 143.53, 141.16, 136.78, 134.13, 131.37, 129.06, 128.63, 126.98, 124.82, 123.78, 38.09, 34.80, 28.09. HRMS: [M+H]+ calc. 259.0793, found 259.0817.

### 5,5-Dimethyl-4, 5-dihydronaphtho[1,2-b]thiophene-2-carboxamide (RU-NT-253)

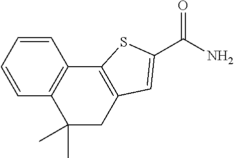 [SMILES: CC1(C)Cc2cc(C(N)=O)sc2-c2ccccc21]

5,5-Dimethyl-4,5-dihydronaphtho[1,2-b]thiophene-2-carboxylic acid (RU-NT-206, 125 mg, 0.42 mmol, 1 equiv.), ammonium chloride (23 mg, 0.42 mmol, 1 equiv.), HATU (194 mg, 0.51 mmol, 1.2 equiv.), diisopropylethylamine (0.15 mL, 0.85 mmol, 2 equiv.) were dissolved in DMF (1.6 mL) in a vial. The reaction mixture was flushed with nitrogen for 5 min. The reaction was left stirring in the heating block (50° C.) and reaction progress was monitored by LC-MS. After the completion (the next day) reaction mixture was concentrated using Biotage V-10. Subsequently, the crude mixture was purified by prep-HPLC to achieve the desired purity (ACCQPrep, C18, liquid injection, (water/formic acid 0.1%)/CH₃CN, 10%-100%). Fractions, that contained the product (by LCMS), were combined to give the desired product as a white solid (71 mg, 65%). ¹H NMR: (500 MHz, DMSO-d₆) δ 7.60 (s, 1H), 7.45-7.38 (m, 2H), 7.29 (td, J=7.5, 1.5 Hz, 1H), 7.25 (td, J=7.5, 1.5 Hz, 1H), 2.70 (s, 2H), 1.24 (s, 6H). ¹³C NMR: (126 MHz, DMSO-d₆) δ 162.94, 143.16, 138.97, 137.49, 136.51, 129.66, 128.96, 128.58, 126.96, 124.77, 123.57, 38.35, 34.89, 28.15. HRMS: C15H16NOS+, [M+H]+ calc. 258.09471, found 258.0957, err. 3.8 ppm.

### 5,5-Dimethyl-4,5-dihydronaphtho[1,2-b]thiophene-2-carbonitrile (RU-NT-254)

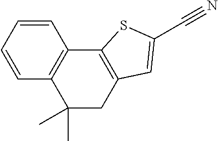 [SMILES: CC1(C)Cc2cc(C#N)sc2-c2ccccc21]

5,5-Dimethyl-4,5-dihydronaphtho[1,2-b]thiophene-2-carboxamide (RU-NT-253, 60 mg, 0.23 mmol, 1 equiv.) was dissolved in dichloromethane (1.75 mL) and methyl N-(triethylammoniumsulfonyl)carbamate (Burgess Reagent, 71 mg, 0.28 mmol, 1.2 equiv.) was added to the mixture. The reaction mixture was flushed with nitrogen for 5 min. The reaction was left stirring at room temperature and reaction progress was monitored by LC-MS. After the completion (the next day) reaction mixture was concentrated using Biotage V-10. Subsequently, the crude mixture was purified by prep-HPLC to achieve the desired purity (ACCQPrep, C18, liquid injection, (water/formic acid 0.1%)/CH₃CN, 10%-100%). Fractions, that contained the product (by LCMS), were combined to give the desired product as a colourless oil (41 mg, 73%). ¹H NMR: (400 MHz, DMSO-d₆) δ 7.85 (s, 1H), 7.47 (dt, J=7.6, 1.3 Hz, 2H), 7.38 (td, J=7.5, 1.5 Hz, 1H), 7.29 (td, J=7.4, 1.3 Hz, 1H), 2.76 (s, 2H), 1.24 (s, 6H). ¹³C NMR: (101 MHz, DMSO-d₆) δ 144.24, 142.81, 140.02, 137.14, 130.35, 128.12, 127.63, 125.48, 124.63, 115.26, 105.19, 38.33, 35.27, 28.47. HRMS: C15H14NS+, [M+H]+ calc. 240.08415, found 240.0851, err. 4.0 ppm.

### General Procedure for Purification Using ACCQPreP

Prep HPLC were performed as (Phenomenex Gemini-NX C18 75*30 mm, 3 m; mobile phase: [water (10 mM NH₄HCO₃)-ACN];B %: 40%-60%,6 min). ACCQPreP HP125, (Waters XBridge BEH C18 100*30 mm10 m; mobile phase: [water(10 mM NH₄HCO₃)-ACN]; B %: 5%-100%, 20 min, Method A). Alternatively, preparative HPLC was performed on ACCQPrep HP125 system, (Waters XBridge BEH C18 column, 100×30 mm, 10 m; mobile phase: A: 0.1% v/v trifluoroacetic acid in water; B: 0.1% v/v trifluoroacetic acid in acetonitrile; 5% to 100%, 20 min, Method B).

### General Procedure for the Synthesis of Substituted Benzothiophenes

To a suspension of NaH (60% in oil, 2 eq) in dry THF (12 M) was added methyl/ethyl 2-mercaptoacetate (1.2 eq) at room temperature and stirred for 30 minutes under nitrogen. To the reaction was added a solution of ketone/aldehyde (1 eq) in THF (0.87 M) and allowed to reflux overnight at 86° C. under nitrogen. The reaction mixture was cooled to room temperature, diluted with EtOAc and brine. The organic layer was dried over anhydrous magnesium sulphate, filtered, and concentrated under reduced pressure. The sample was then purified using C18 reverse phase column (80 g column) to obtain the desired product which was taken directly for saponification using the general procedure (WO2011056725A1_0167).

### General Procedure for Amide Coupling Reactions

Method A: acid chloride formation using thionyl chloride/oxalyl chloride: Step 1: Carboxylic acid (1eq) dissolved in the minimal quantitative amount of DCM in a 100 mL RBF with a reflux condenser and treated with thionyl chloride (1-2 mL), and DMF (1 mL). The solution was heated at reflux overnight and the solvent was reduced in vacuo to remove excess thionyl chloride before being taken directly to the next step.

Step 2: To a stirred solution of aniline (1.1 mmol) in DCM (3 mL) in a 20 mL scintillation vial was added acid chloride (1 eq) and excess TEA (3 eq) in ice. The reaction mixture was left under stirring at rtp overnight then diluted with dichloromethane and washed with a saturated aqueous solution of brine and then dried over anhydrous MgSO₄. The solution was concentrated under reduced pressure, and the crude material was fractionated and purified using preparative HPLC Method B unless otherwise specified to give the desired product after being dried on the V10.

Method B: EDC HCl (1.5 eq) and HOAt/HOBT (1.5 eq) were added to a stirred solution of carboxylic acid (1 eq) and then aniline in EtOAc/DCM/DMF (3-5 mL) and excess TEA (3 eq) at room temperature. The reaction mixture was left under stirring at 90° C. in a 100 mL RBF overnight then diluted with dichloromethane and washed with a saturated aqueous solution of brine and then dried over anhydrous MgSO₄. The solution was concentrated under reduced pressure, and the crude material was fractionated and purified using preparative Method B unless otherwise specified to give the desired product after dryness on the V10.

Method C: In a 100 mL RBF containing carboxylic acid (1 eq), aniline (1.2 eq) and triethyamine (3.0 eq, 1 mL) in EtOAc/DCM/DMF (1-3 mL). The reaction mixture was cooled to 0° C. and T3P (propane phosphonic acid anhydride, 50% solution in EtOAc/DMF, 3 eq) was added to the solution. The reaction was heated overnight at 90° C. overnight. Extraction was carried out 3x using EtOAc and brine and combined organic layers were collected and dried over anhydrous MgSO₄. The organic layers were concentrated under vacuum and purified using preparative HPLC Method B (10.1016/j.bmcl.2012.01.082) unless otherwise specified.

Method D: To a solution of carboxylic acid (1 mmol) in DCM/THF (2 mL), EDC HCl (1.2 eq), aniline (1 eq) was added. After stirring the mixture at 60° C., under reflux overnight, in a 50 mL RBF, the reaction mixture was concentrated under reduced pressure and extracted with EtOAc and brine/water 3 times. The organic layers were combined and dried over anhydrous MgSO₄. The organic layer was reduced in vacuo, and purified using preparative HPLC Method B to give the desired product after reduction in vacuo and further dryness using the V-10.

Method E: PyBOP (1.5 eq) were added to a stirred solution of carboxylic acid (1.56 mmol) and then aniline in DCM (5 mL) and excess DIPEA (1.5 eq) at room temperature. The reaction mixture was left under stirring at 60° C. overnight in a 50 mL RBF then diluted with dichloromethane and washed with a saturated aqueous solution of brine and then dried over anhydrous MgSO₄. The solution was concentrated under reduced pressure; the crude material was fractionated and purified using preparative HPLC Method B to give the desired product after drying on the V-10.

### General Procedure for Suzuki Coupling Reactions Using

Method A: In a 10 mL Biotage MW vial, amide (1 eq), [5-(methoxycarbonyl)thiophen-2-yl]boronic acid (2 eq), 2′-amino-[1,1′-biphenyl]-2-ide dicyclohexyl[2′,4′,6′-tris(propan-2-yl)-[1,1′-biphenyl]-2-yl]phosphane methanesulfonic acid palladium using XPhos PdG3 (Cl, 4 mol %) or bis(triphenylphosphine)palladium(II) dichloride (Pd(PPh₃)₂Cl₂, C2, 5 mol %) were taken up in dry dioxane (0.08 M-0.15 M), 2M Na₂CO₃ (aq) (8 eq) (unless otherwise specified). The reaction was degassed for 3 mins prior to heating overnight at 100-120° C. The mixture was filtered through celite into a separating funnel for extraction, solvent was reduced in vacuo and extraction was carried out 3 times using EtOAc/water and brine. Filtration was to remove insoluble Pd impurities prior to entering the separating funnel. The organic layers were combined and dried over anhydrous MgSO₄, filtered, concentrated. The crude was purified or taken directly for the saponification using the general procedure. Purification was carried out using preparative HPLC Method B unless otherwise specified prior to being concentrated under vacuum and dryness using the V-10 to give the desired product.

Method B: In a 20 mL Biotage MW vial was added substituted thiophene (1 eq), boronic acid (1.5 eq), Pd(PPh₃)₄ (5 mol %) and K₂CO₃ (2 eq) in toluene/ethanol/water (10:3:2, v/v/v). The reaction mixture was degassed under N₂ for 3 mins, The reaction mixture was heated at 110° C. overnight under a nitrogen atmosphere after being degassed for 3 mins. The reaction mixture was cooled to room temperature and diluted with EtOAc. Filtration of the reaction mixture on Celite and the reaction solvent was evaporated to dryness. Purification was carried out using preparative HPLC Method B unless otherwise specified prior to being concentrated under vacuum and dryness using the V-10 to give the desired product.

### General Procedure for N-Alkylation

Method A: To a solution of amide (1 eq) in acetonitrile/acetone/THF/DMF (amount as indicated in the exact procedure) was added potassium carbonate (2 eq), and excess alkyl halide. The mixture was stirred at 70° C., overnight then filtered through Celite. The filtrate was concentrated under reduced pressure, and the crude product was purified by preparative HPLC using Method B unless otherwise specified to give the desired product.

Method B: substituted methyl ester (1 eq) was added to a 20 mL scintillation vial and then dissolved in anhydrous DMF/THF (1 mL). NaH (1.5 eq, 60% dispersion in oil, 1.5-2 eq) was added at once. After gas evolution ceased at this temperature, excess iodomethane was added at once. The reaction was monitored by mass spec, once LCMS analysis indicated full conversion of starting material, the reaction was slowly quenched at 0° C. by the addition of a saturated aqueous solution of ammonium chloride. The layers of the resulting biphasic mixture was separated and aqueous layer was extracted with EtOAc (3×). The combined organic layers were washed with water and brine, dried over MgSO₄, and concentrated in vacuo. Purification was carried out using preparative HPLC Method B to give the desired product unless otherwise specified.

### General Procedure for Saponification of Methyl Esters

To the starting material, (1 eq) in a 20 mL scintillation vial, excess LiOH was added. The reaction was stirred at room temperature in a solution of THF: MeOH: H₂O (1:1:0.5 mL) for 1-2 hours or overnight at room temperature unless otherwise specified. The reaction mixture was then acidified to a pH of 2 with 2M HCl. This was followed by extraction three times with EtOAc and water. The organic layer was reduced under vacuum. The resulting mixture was then purified Purification was carried out using preparative HPLC Method B unless otherwise specified prior to being concentrated under vacuum and overnight lyophilization of all final compounds to give the desired product (WO2020114494 A1 2020-06-11).

### General Procedure for Tetrazole Formation

A solution of nitrile (1 eq) in DMF (1 mL) in a 20 mL scintillation vial was added sodium azide (2.5 eq) and ammonium chloride (1.5 eq) were added and stirring continued at 50° C./120° C. overnight. The mixture was cooled, acidified with 2M HCl and extracted with EtOAc and brine 3 times. The combined organic layers were dried over anhydrous MgSO₄.and solvent was reduced in vacuo. Purification was carried out using preparative HPLC (column size:100×30 mm, Gemini) using 0.1% TFA in CH₃CN (solvent B) and 0.1% TFA in water (solvent A) and the final product was lyophilized overnight.

### Synthesis of methyl 5-{2-[(]E)-2-cyanoeth-1-en-1-yl]phenyl}thiophene-2-carboxylate & methyl 5-{2-[(]Z)-2-cyanoeth-1-en-1-yl]phenyl}thiophene-2-carboxylate

In a 20 mL scintillation vial, methyl 5-(2-formylphenyl)thiophene-2-carboxylate (100 mg, 0.41 mmol), diethylcyanophosphonate (71.93 mg, 1 eq) were dissolved dry DCM (2 mL) and 0.243 g of calcium hydroxide solid catalyst was added. The solvent was reduced under pressure and the reaction mixture was allowed to stand at room temperature for 24 h. After addition of DCM, the mineral salt was filtered off. The solvent was evaporated and product attempted for purification by ACCQPrep. Purification was carried out using preparative HPLC Method B. The products came off as a 1:1 mixture of the cis and trans and were taken to the next step without further purification. LCMS of crude (220 nm, 240 nm): Rt: 2.977 min (cis) 3.020 min (trans), m/z (ESI): 308.35 [M+K]⁺. ¹H NMR (500 MHz, DMSO-d₆) δ 7.87 (dd, J=3.9, 2.8 Hz, 2H), 7.85-7.82 (m, 1H), 7.68-7.63 (m, 1H), 7.62-7.58 (m, 2H), 7.58-7.55 (m, 2H), 7.52 (dd, J=11.8, 0.6 Hz, 1H), 7.27-7.25 (m, 1H), 7.24-7.21 (m, 1H), 6.53-6.47 (m, 1H), 6.04 (dd, J=11.8, 1.1 Hz, 1H), 3.86 (d, J=3.9 Hz, 6H).

### Synthesis of 5-{2-[(]E)-2-cyanoeth-1-en-1-yl]phenyl}thiophene-2-carboxylic acid (RU-NT-248) & 5-{2-[(]Z)-2-cyanoeth-1-en-1-yl]phenyl}thiophene-2-carboxylic acid (RU-NT-249)

According to the general procedure for saponification of methyl esters. In a 4 mL scintillation vial, methyl 5-{2-[(1Z)-2-cyanoeth-1-en-1-yl]phenyl}thiophene-2-carboxylate (76 mg white solid, 0.1 mmol), 5 hrs at 40° C. Solvent was reduced under vacuo, addition of 2N HCl until a pH of 4-5 was reached and extraction 3 x using EtOAc was carried out. Organic layers were combined and dried over anhydrous MgSO₄. Prep HPLC was carried out using gradient 55-60% of 0.1% TFA in ACN using ChemPurePrep column (150×30 mm size column) according to Method B for purification to give the desired products as white solids in a 1:1 ratio upon separation. Trans isomer (16.30 mg, 44% yield):¹H NMR (500 MHz, DMSO-d₆) δ 7.85 (d, J=6.8 Hz, 1H), 7.78 (d, J=3.7 Hz, 1H), 7.64 (d, J=16.6 Hz, 1H), 7.58-7.56 (m, 2H), 7.56-7.51 (m, 1H), 7.20 (d, J=3.7 Hz, 1H), 6.49 (d, J=16.6 Hz, 1H). LCMS (Rt=2.467 min), m/z: 256.29 [M+H]⁺·HR-MS (ESI) m/z: [M+H]+ Calcd for C14H9NO₂S 256.0426; Found 256.0428; error 0.78 ppm. Cis isomer (15.00 mg, 44% yield): ¹H NMR (500 MHz, DMSO-d₆) δ 7.89-7.81 (m, 1H), 7.75 (d, J=3.8 Hz, 1H), 7.69-7.62 (m, 1H), 7.62-7.55 (m, 2H), 7.52 (d, J=11.8 Hz, 1H), 7.21 (d, J=3.9 Hz, 1H), 6.03 (d, J=11.8 Hz, 1H). t_{R} 2.420 min, purity ≥95%, m/z (ESI): 256.29 [M+H]⁺.

### Synthesis of 5-[2-ethyl-6-(5-methylthiophene-2-carbonyl)phenyl]thiophene-2-carboxylic acid (RU-NT-274)

According to the general procedure for the saponification of methyl esters. Methyl 5-[2-ethyl-6-(5-methylthiophene-2-carbonyl)phenyl]thiophene-2-carboxylate (40 mg, 0.11 mol), THF (2 mL), LiOH in water (0.5 mL) was added. The resulted suspension becomes transparent after the addition of 0.5 mL MeOH. The rxn was stirred at rtp overnight and monitored by LCMS. Purification was carried out using preparative HPLC Method B. Solvent was concentrated under vacuo to give the desired product as a white solid.

### Synthesis of Methyl 5-{2-ethyl-6-[(5-methylthiophen-2-yl)methyl]phenyl}thiophene-2-carboxylate (RU-NT-277)

To a solution of methyl 5-(2-ethyl-6-(hydroxy(5-methylthiophen-2-yl)methyl)phenyl)thiophene-2-carboxylate (280 mg, 0.75 mmol) in MeCN (6 mL) was added BF₃Et₂O (320 mg, 2.25 mmol) and Et₃SiH (262 mg, 2.25 mmol). Then the reaction mixture was heated to 60° C. for 2 hours. TLC (Petroleum ether: ethyl acetate=5:1, R_{f}(R¹)=0.30, R_{f}(P1)=0.40) showed that the starting material was consumed, and a new spot was detected. The mixture was diluted with saturated aq. NaHCO₃ (10 mL) and extracted with ethyl acetate (20 mL×3). The organic phase was concentrated under reduced pressure. The crude product was purified by column chromatography (SiO₂, ethyl acetate: Petroleum ether=0-15%) to give the desired product as a yellow oil (140 mg, 50% yield). LCMS (220 nm, 254 nm): t_{R} 1.610 min, purity≥95%, m/z (ESI): 357.0 [M+H]⁺. HPLC: RT=4.057 min, Area=95.58%. ¹H NMR (400 MHz, DMSO-d₆) δ 7.83 (d, J=3.6 Hz, 1H), 7.37 (t, J=7.6 Hz, 1H), 7.25 (d, J=6.8 Hz, 1H), 7.18 (d, J=7.6 Hz, 1H), 6.98 (d, J=3.6 Hz, 1H), 6.57-6.50 (m, 1H), 6.38 (d, J=3.2 Hz, 1H), 3.91-3.79 (m, 5H), 2.39 (q, J=7.6 Hz, 2H), 2.33 (s, 3H), 1.04 (t, J=7.6 Hz, 3H).

### Synthesis of 5-{2-ethyl-6-[(5-methylthiophen-2-yl)methyl]phenyl}thiophene-2-carboxylic acid (RU-NT-281)

According to the general procedure for saponification of methyl esters. Methyl 5-{2-ethyl-6-[(5-methylthiophen-2-yl)methyl]phenyl}thiophene-2-carboxylate (46 mg, 0.13 mmol), rtp, O/N. Purification was carried out using preparative HPLC Method B. Solvent was concentrated under vacuo to give the desired product as a white solid (31 mg, 31% yield). LCMS (220 nm, 254 nm): t_{R} 3.545 min, purity ≥95%, m/z (ESI): 343.16 [M+H]⁺. HRMS m/z (ESI): [M+H]⁺ Calcd for C₁₉H₁₈O₂S₂ 343.0820; Found 343.0820; error 0.00 ppm. ¹H NMR (500 MHz, DMSO-d₆) δ 7.74 (d, J=3.7 Hz, 1H), 7.36 (t, J=7.7 Hz, 1H), 7.24 (dd, J=7.8, 1.3 Hz, 1H), 7.17 (dd, J=7.7, 1.3 Hz, 1H), 6.95 (d, J=3.7 Hz, 1H), 6.54 (dd, J=3.4, 1.2 Hz, 1H), 6.39 (d, J=3.3 Hz, 1H), 3.83 (s, 2H), 2.39 (q, J=7.5 Hz, 2H), 2.33 (d, J=1.2 Hz, 3H), 1.04 (t, J=7.5 Hz, 3H). LCMS (ESI) m/z: 343.15 [100%, M+H]⁺.

### Synthesis of (2-bromo-3-ethylphenyl)(5-methylthiophen-2-yl)methanone

To a solution of 2-bromo-3-ethyl-N-methoxy-N-methylbenzamide (2.28 g, 8.40 mmol) in THF (20 mL) was added (5-methylthiophen-2-yl)magnesium bromide (2.03 g, in 20 mL THF) at 0° C. Then the reaction mixture was stirred at 20° C. for 2 hours. LCMS showed that ~53% start material was remained, and ~46% desired product was detected. The mixture was concentrated under reduced pressure. The crude product was purified by column chromatography (SiO₂, ethyl acetate: Petroleum ether=0-10%) to give the desired product as a yellow oil (1.90 g, 69.1% yield). LCMS (220 nm, 254 nm): t_{R} 1.227 min, purity ≥95%, m/z (ESI): 310.9 [M+H]⁺. ¹H NMR (400 MHz, DMSO-d₆) δ 7.50 (dd, J=7.6, 2.0 Hz, 1H), 7.45 (t, J=7.6 Hz, 1H), 7.30 (dd, J=7.2, 1.6 Hz, 1H), 7.19 (d, J=3.6 Hz, 1H), 6.95 (dd, J=3.6, 0.8 Hz, 1H), 2.78 (q, J=7.6 Hz, 2H), 2.55 (s, 3H), 1.20 (t, J=7.6 Hz, 3H).

### Synthesis of methyl 5-(2-ethyl-6-(5-methylthiophene-2-carbonyl)phenyl)thiophene-2-carboxylate

To a mixture of (2-bromo-3-ethylphenyl)(5-methylthiophen-2-yl)methanone (700 mg, 2.26 mmol) and (5-(methoxycarbonyl)thiophen-2-yl)boronic acid (505 mg, 2.71 mmol) in 1,4-dioxane/H₂O (v/v=4/1, 15 mL) was added K₂CO₃ (626 mg, 4.53 mmol) and Pd(dtbpf)Cl2 (146 mg, 0.226 mmol). Then the reaction mixture was heated to 80° C. and stirred under N₂ for 12 hours. LCMS showed that starting material was consumed, and the desired product was detected as a major peak. The mixture was filtered, and the filtrate was diluted with saturated aq. NH₄C₁ (20 mL) and extracted with ethyl acetate (20 mL×3). The organic phase was concentrated under reduced pressure. The crude product was purified by column chromatography (SiO₂, ethyl acetate: Petroleum ether=0-10%) to give the desired product as a yellow oil (760 mg, 86% yield). LCMS (220 nm, 254 nm): t_{R} 1.239 min, purity ≥95%, m/z (ESI): 371.0 [M+H]+. HPLC: RT=4.684 min, Area=98.38%. ¹H NMR (400 MHz, DMSO-d₆) δ 7.69 (d, J=4.0 Hz, 1H), 7.59-7.50 (m, 2H), 7.38 (dd, J=7.2, 1.6 Hz, 1H), 7.23 (d, J=4.0 Hz, 1H), 6.97 (d, J=4.0 Hz, 1H), 6.90 (dd, J=4.0, 1.2 Hz, 1H), 3.79 (s, 3H), 2.56 (q, J=7.6 Hz, 2H), 2.49 (s, 3H), 1.09 (t, J=7.6 Hz, 3H).

### Synthesis of methyl 5-(2-ethyl-6-(hydroxy(5-methylthiophen-2-yl)methyl)phenyl)thiophene-2-carboxylate

To a solution of methyl 5-(2-ethyl-6-(5-methylthiophene-2-carbonyl)phenyl)thiophene-2-carboxylate (500 mg, 1.35 mmol) in MeOH (10 mL) was added NaBH₄ (102 mg, 2.70 mmol) at 0° C. and stirred at 0° C. for 3 hours. LCMS showed that the starting material was consumed, and the desired product was detected as a major peak. The mixture was quenched with water (1 mL) and concentrated under reduced pressure. The crude product was purified by column chromatography (SiO₂, ethyl acetate: Petroleum ether=0-15%) to give the desired product as a yellow oil (376 mg, 71% yield). LCMS (220 nm, 254 nm): t_{R} 1.354 min, purity ≥95%, m/z (ESI): 355.0 [M+H]⁺. ¹H NMR (400 MHz, DMSO-d₆) δ 7.82 (s, 1H), 7.51 (d, J=7.6 Hz, 1H), 7.44 (t, J=7.6 Hz, 1H), 7.28 (d, J=7.6 Hz, 1H), 6.52 (d, J=2.4 Hz, 1H), 6.29 (d, J=3.2 Hz, 1H), 5.99 (d, J=4.0 Hz, 1H), 5.54 (s, 1H), 3.83 (s, 3H), 2.44-2.30 (m, 5H), 1.04 (t, J=7.6 Hz, 3H).

### Synthesis of {3-ethyl-2-[5-(hydroxymethyl)thiophen-2-yl]phenyl}(5-methylthiophen-2-yl)methanol (RU-NT-282)

To a solution of methyl 5-(2-ethyl-6-(hydroxy(5-methylthiophen-2-yl)methyl)phenyl)thiophene-2-carboxylate (35.0 mg, 0.094 mmol) in MeOH (1 mL) was added Pd/C (1.33 mg, 0.01 mmol) and stirred under H2 (15 Psi) at 20° C. for 2 hours. TLC (Petroleum ether: ethyl acetate=3:1, R_{f}(R1)=0.50, R_{f}(P1)=0.20) showed that the starting material was consumed, and a new spot was detected. The mixture was filtered, and the filtrate was concentrated under reduced pressure. Purification was carried out using preparative HPLC Method B. Solvent was concentrated under vacuo prior to lyophilization giving the desired product as a colorless oil (15 g, 41% yield). LCMS (220 nm, 254 nm): t_{R} 1.180 min, purity ≥95%, m/z (ESI): 327.1 [M-OH]+. ¹H NMR (500 MHz, DMSO-d₆) δ 7.45 (d, J=8.8 Hz, 1H), 7.38 (t, J=7.7 Hz, 1H), 7.23 (dd, J=7.6, 1.4 Hz, 1H), 6.91 (d, J=24.5 Hz, 1H), 6.52 (dd, J=3.4, 1.1 Hz, 1H), 6.30 (d, J=3.5 Hz, 1H), 5.90 (d, J=4.4 Hz, 1H), 5.60 (s, 1H), 5.49 (t, J=5.7 Hz, 1H), 4.65 (d, J=5.8 Hz, 2H), 2.41 (q, J=7.5 Hz, 2H), 2.34 (d, J=1.1 Hz, 3H), 1.04 (t, J=7.5 Hz, 3H).

### Synthesis of methyl 5-(2-formylphenyl)thiophene-2-carboxylate

In a 100 mL three-neck flask, a mixture was prepared that included methyl 5-bromothiophene-2-carboxylate (lg, 5.5 mmol), 2-formylphenyl)boronic acid (0.88 g, 5.8 mmol), along with Na₂CO₃ (2 eq). The flask was then filled with toluene (10 mL), ethanol (5 mL), and water (5 mL), and the entire setup was placed under a nitrogen atmosphere while stirring at room temperature. Tetrakis(triphenylphosphine)palladium (0) (46 mg) was subsequently added to the mixture. The resulting mixture was heated to 80° C. and stirred for five hours. Upon completion of the reaction, the organic layer was extracted with toluene two to three times with brine. The organic layers were dried over anhydrous magnesium sulfate, followed by purification through silica gel flash purification using a 80 g column using a normal phase 0-10% gradient of ethyl acetate in hexane. The final purified fraction yielded a white solid, obtained by washing with methanol (0.5 mL) and acetonitrile (0.5 mL) giving the desired product as a white solid (0.4 g, 36% yield). ¹H NMR (500 MHz, DMSO-d₆) δ 10.08 (d, J=0.8 Hz, 1H), 7.95 (dd, J=8.3, 1.6 Hz, 1H), 7.86 (d, J=3.8 Hz, 1H), 7.79 (td, J=8.3, 1.2 Hz, 1H), 7.66 (td, J=7.6 Hz, 1H), 7.38 (d, J=3.8 Hz, 1H), and 3.86 (s, 3H). LCMS (220 nm, 254 nm): t_{R} 2.821 min, purity=89% %, m/z (ESI): 247.10 [M+H]⁺.

### Synthesis of 5-(2-formylphenyl)thiophene-2-carboxylic acid (RU-NT-285)

In a 4 mL scintillation vial, SM (40 mg, 0.16 mmol) was dissolved in a mixture of dry dioxane (1.5 mL) and 1N NaOH (1.5 mL) in a 1:1 ratio. The reaction mixture was stirred for 30 mins at rtp until completion of the reaction as observed by LCMS. The aqueous layer was acidified with 6N hydrochloric acid, and extracted with EtOAc (3 x) and brine. The combined organic layers were dried over anhydrous MgSO₄ were reduced in vacuo. Purification was carried out using preparative HPLC Method B. Solvent was concentrated under vacuo to give the desired product as white crystals (33.4 mg, 89% yield). HR-MS (ESI) m/z: [M+H]+ Calcd for C₁₂H₈O₃S 233.0266; Found 233.0266; error 0.00 ppm. ¹H NMR (500 MHz, DMSO-d₆) δ 13.32 (br s, 1H), 10.09 (S, 1H), 7.94 (dt, J=7.9, 1.1 Hz, 1H), 7.87-7.73 (m, 2H), 7.71-7.60 (m, 2H), 7.34 (dd, J=3.8, 0.9 Hz, 1H).

### Synthesis of methyl 5-(2-ethylphenyl)-4-methylthiophene-2-carboxylate

According to general procedure A for Suzuki coupling reactions, precatalyst Cl. Methyl 5-bromo-4-methylthiophene-2-carboxylate (100 mg, 0.42 mmol), (2-ethylphenyl)boronic acid (76.5 mg, 1.2 eq), dry dioxane (5 mL). The reaction mixture was heated in the microwave 10 min at 130° C. The mixture was partitioned in EtOAc/water and extraction was carried out 3 times. Filtered through cotton into a sep funnel for extraction. Filtration was to remove insoluble Pd impurities prior to entering the sep funnel. Dried combined organic over MgSO₄, filtered, concentrated. Purification was carried out using preparative HPLC Method B. Solvent was concentrated under vacuo to give the desired product as a white solid (50 mg, 45% yield). LCMS (220 nm, 254 nm): t_{R} 3.673 min, m/z (ESI): 302.15 [M+CH₃CN]⁺.

### Synthesis of 5-(2-ethylphenyl)-4-methylthiophene-2-carboxylic acid (RU-NT-286)

According to the general procedure for the saponification of methyl ester. SM (50 mg, 0.19 mmol), 40° C. O/N. Purification was carried out using preparative HPLC Method B. Solvent was concentrated under vacuo to give the desired product as a white solid (20 mg, 43% yield). HR-MS (ESI) m/z: [M+H]+ Calcd for C₁₄H₁₄O₂S 247.0787; Found 247.0787; error 0.00 ppm. ¹H NMR (500 MHz, DMSO-d₆) δ 13.01 (s, 1H), 7.75-7.52 (m, 1H), 7.42-7.33 (m, 2H), 7.25 (td, J=8.8, 2.1 Hz, 1H), 7.22-7.15 (m, 1H), 2.62-2.34 (m, 3H), 1.95 (s, 3H), 1.02 (m, J=7.5, 2.8, 1.4 Hz, 3H).

### Synthesis of methyl 5-(2-ethylphenyl)-1-methyl-1H-pyrazole-3-carboxylate

According to general method B for Suki coupling reactions. 5-Bromo-1-methyl-1H-pyrazole-3-carboxylate (0.11 g, 1 eq.) dry THF (2 mL), Pd(PPh₃)₄ (5 mol %) and (2-ethylphenyl)boronic acid (1.5 eq.), 2 M Na₂CO₃ (0.8 mL, 3 eq). The mixture was heated to reflux overnight. Purification was carried out using preparative HPLC Method A. Solvent was concentrated under vacuo to give the desired product as a white solid (79.3 mg, 64% yield). LCMS (220 nm, 254 nm): t_{R} 2.720 min, m/z (ESI): 245.15 [M+H]⁺.

### Synthesis of 5-(2-ethylphenyl)-1-methyl-1H-pyrazole-3-carboxylic acid (RU-NT-287)

According to the general procedure for the saponification of methyl esters. Methyl 5-(2-ethylphenyl)-1-methyl-1H-pyrazole-3-carboxylate (53.2 mg, 0.22 mmol). Purification was carried out using preparative HPLC Method B. Solvent was concentrated under vacuo prior to lyophilization giving the desired product as a white solid (11.9 mg, 26% yield). LCMS (220 nm, 254 nm): t_{R} 2.244 min, purity ≥95%, m/z (ESI): 231.15 [M+H]⁺. ¹H NMR (300 MHz, DMSO-d₆) δ 12.65 (brs, 1H), 7.44 (d, J=8.0 Hz, 2H), 7.28 (dd, J=14.0, 7.7 Hz, 2H), 6.66 (s, 1H), 3.61 (s, 3H), 2.45 (q, J=6.8 Hz, 2H), 1.01 (t, J=7.6 Hz, 3H).

### Synthesis of methyl 5-bromo-1-(oxan-2-yl)-1H-pyrazole-3-carboxylate

In a 20 mL scintillation vial, dihydropyran (1.67 mL, 3 eq) was added to a solution of methyl 5-bromo-1H-pyrazole-3-carboxylate (1.25 g, 6.09 mmol) and PTSA (0.12 g, 00.1 eq) in DCM (5 mL). Reaction was stirred overnight at rtp. Extraction using DCM and brine was carried out 3 x. The combined organic layers were dried over anhydrous MgSO₄ and reduced under pressure. Flash purification was carried out using 10% EtOAc in hexane using a 25 g silica column to give the desired product a white solid (0.5 g, 28% yield). LCMS (220 nm, 254 nm): t_{R} 2.719 min, purity ≥95%, m/z (ESI): 328.40 [M+K]⁺. ¹H NMR (300 MHz, DMSO-d₆) δ 7.06 (s, 1H), 6.09 (dd, J=9.8, 2.4 Hz, 1H), 3.85 (s, 2H), 3.68-3.46 (m, 1H), 2.35-2.02 (m, 1H), 2.07-1.79 (m, 2H), 1.81-1.33 (m, 6H).

### Synthesis of methyl 5-bromo-1-(oxan-2-yl)-1H-pyrazole-3-carboxylate

In a 20 mL dram vial, dihydropyran (1.67 mL, 3.0 eq) was added to a solution of methyl 5-bromo-1H-pyrazole-3-carboxylate (1.25 g, 6.09 mmol) and PTSA (0.12 g, 00.1 eq) in 5 mL of DCM. Reaction was stirred overnight at rtp.Extraction using DCM and brine was carried out 3 x. The combined organic layers were dried over anhydrous MgSO₄ and reduced under pressure.Flash purification was carried out using 10% EtOAc in hexane using a 25 g column to give the desired product a a white solid (0.5 g, 28% yield). ¹H NMR (300 MHz, DMSO-d₆) δ 7.06 (s, 1H), 6.09 (dd, J=9.8, 2.4 Hz, 1H), 3.85 (s, 2H), 3.68-3.46 (m, 1H), 2.35-2.02 (m, 1H), 2.07-1.79 (m, 2H), 1.81-1.33 (m, 6H). LCMS (Rt=2.719 min), m/z: 328.40 [M+K]⁺.

### Synthesis of methyl 5-(2-ethylphenyl)-1-(oxan-2-yl)-1H-pyrazole-3-carboxylate

To a stirred solution of methyl 5-bromo-1-(oxan-2-yl)-1H-pyrazole-3-carboxylate (0.13 g, 0.45 mmol, 1.2 eq) in dry THF (4 mL) and water (1.2 mL) was added in a 20 mL scintillation vial. (2-Ethylphenyl)boronic acid (0.1 g, 0.67 mmol), potassium phosphate, tribasic (0.28 g, 2 eq) under nitrogen purging for 10 min at room temperature. Then PdCl₂(PPh₃)₂ (0.05 eq) was added and the reaction mixture was heated at 90° C. for 16 h. The reaction mixture was quenched with water, filtered through cotton wool, and extracted three times with ethyl acetate. The organic layer was dried over by MgSO₄ and concentrated under reduced pressure. Purification was carried out using preparative HPLC Method A. Solvent was concentrated under vacuo to give the desired product as a colorless oil (80.1 mg, 38% yield). LCMS (220 nm, 254 nm): t_{R} 3.567 min, purity ≥95%, m/z (ESI): 315 [M+H]⁺. ¹H NMR (500 MHz, CDCl₃) δ 7.74 (dq, J=7.4, 1.2 Hz, 1H), 7.54 (dt, J=4.9, 1.4 Hz, 2H), 7.47 (dtd, J=8.8, 3.3, 2.3 Hz, 1H), 7.26 (d, J=1.6 Hz, 1H), 6.57 (dt, J=9.7, 2.2 Hz, 1H), 4.42-4.20 (m, 1H), 4.16 (s, 2H), 4.01 (ddt, J=13.0, 11.1, 2.1 Hz, 1H), 3.27-2.97 (m, 2H), 2.87-2.68 (m, 1H), 2.39 (dt, J=13.3, 4.8 Hz, 1H), 2.31-2.19 (m, 1H), 2.04-1.90 (m, 2H), 1.84 (t, J=9.1 Hz, 1H), 1.45 (td, J=7.5, 1.6 Hz, 3H).

### Synthesis of 5-(2-ethylphenyl)-1H-pyrazole-3-carboxylic acid (RU-NT-288)

According to the general procedure for saponification of methyl ester. Methyl 5-(2-ethylphenyl)-1H-pyrazole-3-carboxylate (60.0 mg, 0.26 mmol). Purification was carried out using preparative HPLC Method A. Solvent was concentrated under vacuo to give the desired product as a white solid (40 mg, 72% yield). LCMS (220 nm, 254 nm): t_{R} 2.249 min, purity ≥95%, m/z (ESI): 238 [M+Na]⁺. ¹H NMR (500 MHz, DMSO-d₆) δ 7.42 (d, J=7.6 Hz, 1H), 7.32 (t, J=2.9 Hz, 1H), 7.26 (d, J=4.5 Hz, 2H), 6.74 (s, 1H), 2.76 (br q, 1H), 1.91 (q, J=1.7 Hz, 2H), 1.10 (t, J=1.8 Hz, 3H).

### Synthesis of 5-(2,6-diethylphenyl)-N-hydroxythiophene-2-carboxamide (RU-NT-289)

In a 20 mL scintillation vial was added hydroxylamine (0.5 g in 1 mL water) dropwise to a solution of methyl 5-methyl 5-(2,6-diethylphenyl)thiophene-2-carboxylate (33 mg, 0.12 mmol) in THF (1 mL) and water (1 mL). Add an aqueous solution of NaOH (2 M, 0.2 g in 0.4 mL water) until alkaline the solution (pH=11). The reaction mixture was stirred for 24 hours at room temperature. Based on the LCMS, the mixture was then heated at 35° C. for 3-4 hours. The mixture was diluted with aqueous layers were combined and dried over anhydrous MgSO₄ before concentrating in vacuo. Purification was carried out using preparative HPLC Method B. Solvent was concentrated under vacuo to give the desired product as a white solid (10 mg, 30% yield). LCMS (220 nm, 254 nm): t_{R} 2.664 min, purity ≥95%, m/z (ESI): 276.15 [M+H]⁺. ¹H NMR (300 MHz, DMSO-d₆) δ 11.26 (s, 1H), 9.15 (s, 1H), 7.63 (d, J=7.5 Hz, 1H), 7.31 (d, J=7.5 Hz, 1H), 7.17 (d, J=7.6 Hz, 2H), 6.96 (t, J=3.8 Hz, 1H), 2.37 (q, J=7.6 Hz, 5H), 1.03 (t, J=7.5 Hz, 7H).

### Synthesis of methyl 5-(2,6-diethylphenyl)thiophene-2-carboxylate

According to the general procedure for Suzuki coupling reactions using Method A, precatalyst C1. [5-(Methoxycarbonyl)thiophen-2-yl]boronic acid (460 mg, 2.47 mmol), 2-bromo-1,3-diethylbenzene (440 mg, 2.06 mmol), dry dioxane (10 mL). The mixture was heated in the microwave 10 min at 130° C. The mixture was filtered through cotton into a sep funnel for extraction, solvent was reduced in vacuo and extraction was carried out 3 times using EtOAc/water and brine. Filtration was to remove insoluble Pd impurities prior to entering the separating funnel. The organic layers were combined and dried over anhydrous MgSO₄, filtered, concentrated. Purification was carried out using preparative HPLC Method B. Solvent was concentrated under vacuo to give the desired product as a colorless oil (190 mg, 34% yield). LCMS (220 nm, 254 nm): t_{R} 3.823 min, purity ≥95%, m/z (ESI): 316.25 [M+CH₃CN]⁺. ¹H NMR (300 MHz, DMSO-d₆) δ 8.00 (d, J=3.7 Hz, 1H), 7.50 (t, J=7.0 Hz, 1H), 7.33 (d, J=7.6 Hz, 2H), 7.21 (d, J=3.7 Hz, 1H), 3.98 (s, 3H), 2.51 (q, J=7.5 Hz, 4H), 1.18 (t, J=7.5 Hz, 6H).

### Synthesis of 5-(2,6-diethylphenyl)-N-methoxythiophene-2-carboxamide (RU-NT-290)

In a 20 mL scintillation vial add O-methylhydroxylamine (0.5 g in 1 mL water) dropwise to a solution of methyl 5-(2,6-diethylphenyl)thiophene-2-carboxylate (33 mg, 0.12 mmol) in THF (1 mL) and water (1 mL). Add an aqueous solution of NaOH (2 M, 0.2 g in 0.4 mL water) until alkaline the solution (pH=11). Stir the reaction mixture for 24 hours at room temperature. Based on the LCMS, the mixture was then heated at 35° C. for 3-4 hours. Dilute the mixture with aqueous HCl (1 M) until neutral pH value. The organic layers were combined and dried over anhydrous MgSO₄ before concentrating in vacuo. Purification was carried out using preparative HPLC Method B. Solvent was concentrated under vacuo to give a white solid (10 mg, 29% yield). LCMS (220 nm, 254 nm): t_{R} 3.823 min, purity ≥95%, m/z (ESI): 290.39 [M+H]⁺. ¹H NMR (300 MHz, DMSO-d₆) δ 13.14 (s, 1H), 7.74 (d, J=4.0 Hz, 1H), 7.31 (d, J=8.0 Hz, 1H), 7.16 (d, J=7.7 Hz, 2H), 7.01 (d, J=4.0 Hz, 1H), 2.48 (s, 3H), 2.35 (q, J=7.8 Hz, 5H), 1.02 (t, J=4.6 Hz, 6H).

### Synthesis of 5-(5-bromo-2-methylphenyl)thiophene-2-carboxylic acid (RU-NT-293)

To a solution of methyl 5-(5-bromo-2-methylphenyl)thiophene-2-carboxylate (120 mg, 0.385 mmol) in THF (1 mL) and MeOH (2 mL) was added NaOH (1.2 mL, 1 M), the mixture was stirred at 25° C. for 16 hours. TLC (Petroleum ether: ethyl acetate=1: 1, R_{f}(R1)=0.90, R_{f}(P1)=0.20) showed that the starting material was consumed, and a new spot was detected. The mixture was adjusted to pH=5 with aq. HCl (5 mL, 1M) and extracted with ethyl acetate (5 mL×3), dried over anhydrous Na₂SO₄, then filtered and concentrated under reduced pressure to remove the solvent. The crude product was purified by prep-HPLC (column: Gemini, Mobile phase: acetonitrile/H₂O (0.1% FA),) to give the desired compound as a white solid (78.18 mg, 68% yield). LCMS (220 nm, 254 nm): t_{R} 1.196 min, purity ≥95%, m/z (ESI): 294.80 [M−H]⁻. HPLC: RT=3.573 min, Area=99.8%. ¹H NMR (400 MHz, DMSO) δ 13.23 (s, 1H), 7.74 (d, J=4.0 Hz, 1H), 7.61 (d, J=2.0 Hz, 1H), 7.53 (dd, J=8.0, 2.0 Hz, 1H), 7.38-7.24 (m, 2H), 2.36 (s, 3H).

### Synthesis of 5-(2-methyl-5-(phenylethynyl)phenyl)thiophene-2-carboxylic acid (RU-NT-294)

To a solution of methyl 5-(2-methyl-5-(phenylethynyl)phenyl)thiophene-2-carboxylate (140 mg, 0.421 mmol) in THF (1 mL) and MeOH (2 mL) was added NaOH (1.3 mL, 1 M), the mixture was stirred at 25° C. for 16 hours. LCMS showed that the starting material was consumed and ~100% desired product was detected. The mixture was adjusted to pH=5 with citric acid (5 mL, 5% wt) and extracted with ethyl acetate (5 mL×3), dried over anhydrous Na₂SO₄, then filtered and concentrated under reduced pressure to remove the solvent. The crude product was purified by prep-HPLC (column: Gemini, Mobile phase: acetonitrile/H₂O (0.1% FA),) to give the desired product as a white solid (89.84 mg, 99.5% purity, 66.7% yield). LCMS (220 nm, 254 nm): t_{R} 1.350 min, purity ≥95%, m/z (ESI): 316.90 [M−H]⁻. HPLC: RT=4.706 min, Area=99.5%. ¹H NMR (400 MHz, DMSO-d₆) δ 13.18 (s, 1H), 7.76 (d, J=3.6 Hz, 1H), 7.65-7.60 (m, 1H), 7.59-7.55 (m, 2H), 7.52 (dd, J=8.0, 1.6 Hz, 1H), 7.45-7.41 (m, 4H), 7.34 (d, J=3.6 Hz, 1H), 2.43 (s, 3H).

### Synthesis of methyl (E)-5-(2-methyl-5-styrylphenyl)thiophene-2-carboxylate

To a solution of methyl 5-(5-bromo-2-methylphenyl)thiophene-2-carboxylate (180 mg, 0.578 mmol) and (E)-styrylboronic acid (111 mg, 0.751 mol) in 1,4-dioxane/H₂O (4 mL, v/v=5:1) was added K₂CO₃ (120 mg, 0.867 mmol) and Pd(dppf)Cl₂ DCM (47.2 mg, 0.0570 mol), the mixture was stirred under nitrogen atmosphere at 80° C. for 16 hours. LCMS showed that the starting material was consumed and ~64% desired product was detected. The mixture was concentrated under reduced pressure to remove the solvent. The crude product was purified by column chromatography (SiO₂, Ethyl acetate/Petroleum ether=0-10%). The desired compound was obtained as yellow oil (150 mg, 65% yield). ¹H NMR (400 MHz, DMSO-d₆) δ 7.86 (d, J=4.0 Hz, 1H), 7.68 (d, J=1.6 Hz, 1H), 7.61 (d, J=7.2 Hz, 3H), 7.45-7.34 (m, 4H), 7.33-7.24 (m, 3H), 3.86 (s, 3H), 2.41 (s, 3H).

### Synthesis of (E)-5-(2-methyl-5-styrylphenyl)thiophene-2-carboxylic acid (RU-NT-295)

To a solution of methyl (E)-5-(2-methyl-5-styrylphenyl)thiophene-2-carboxylate (140 mg, 0.418 mmol) in THF (1 mL) and MeOH (2 mL) was added NaOH (1.3 mL, 1M), the mixture was stirred at 25° C. for 16 hours. LCMS showed that the starting material was consumed and ~97% desired product was detected. The mixture was adjusted to pH=5 with aq. HCl (5 mL, 1M) and extracted with ethyl acetate (5 mL×3), dried over anhydrous Na₂SO₄, then filtered and concentrated under reduced pressure to remove the solvent. The crude product was purified by prep-HPLC (column: Gemini, Mobile phase: acetonitrile/H₂O (0.1% FA),) to give the desired compound as a white solid (97.00 mg, 72% yield). LCMS (220 nm, 254 nm): t_{R} 1.249 min, purity ≥95%, m/z (ESI): 318.90 [M−H]⁻. HPLC: RT=4.706 min, Area=99.2%. ¹H NMR (400 MHz, DMSO-d₆ δ 13.17) (s, 1H), 7.77 (d, J=3.6 Hz, 1H), 7.69-7.66 (m, 1H), 7.63-7.58 (m, 3H), 7.41-7.35 (m, 3H), 7.34-7.29 (m, 3H), 7.26 (d, J=7.2 Hz, 1H), 2.41 (s, 3H).

### Synthesis of methyl 5-(2-methyl-5-phenethylphenyl)thiophene-2-carboxylate

To a solution of compound methyl 5-(2-methyl-5-(phenylethynyl)phenyl)thiophene-2-carboxylate (160 mg, 0.481 mol) in MeOH (4 mL) was added Pd/C (22.8 mg, 0.0960 mmol) at 25° C., the mixture was stirred under hydrogen atmosphere at 25° C. for 2 hours. LCMS showed that the starting material was consumed and ~98% desired product was detected. The mixture was filtered through a celite pad. the filtrate diluted with MeOH (15 mL) and concentrated under reduced pressure. The desired product was obtained as light-yellow oil (150 mg, 91% yield). LCMS (220 nm, 254 nm): t_{R} 1.810 min, purity ≥95%, m/z (ESI): N/A. ¹H NMR (400 MHz, DMSO-d₆) δ 7.82 (d, J=4.0 Hz, 1H), 7.35-7.12 (m, 9H), 3.84 (s, 3H), 2.88 (s, 4H), 2.36 (s, 3H).

### Synthesis of 5-(2-methyl-5-phenethylphenyl)thiophene-2-carboxylic acid (RU-NT-296)

To a solution of methyl 5-(2-methyl-5-phenethylphenyl)thiophene-2-carboxylate (150 mg, 0.445 mmol) in THF (1 mL) and MeOH (2 mL) was added NaOH (1.3 mL, 1M), the mixture was stirred at 25° C. for 16 hours. LCMS showed that the starting material was consumed and ~100% desired product was detected. The mixture was adjusted to pH=5 with citric acid (5 mL, 1M) and extracted with ethyl acetate (5 mL×3), dried over anhydrous Na₂SO₄, then filtered and concentrated under reduced pressure to remove the solvent. The crude product was purified by prep-HPLC (column: Gemini, Mobile phase: acetonitrile/H₂O (0.1% FA),) to give the desired product as a white solid (93.01 mg, 65% yield). LCMS (220 nm, 254 nm): t_{R} 1.348 min, purity ≥95%, m/z (ESI): 320.95 [M−H]⁻. HPLC: RT=4.140 min, Area=99.8%. H NMR (400 MHz, DMSO-d₆) δ 13.10 (s, 1H), 7.72 (d, J=3.6 Hz, 1H), 7.31-7.16 (m, 9H), 2.88 (s, 4H), 2.36 (s, 3H).

### Synthesis of methyl 5-(5-benzyl-2-methylphenyl)thiophene-2-carboxylate

To a solution of compound methyl 5-(5-bromo-2-methylphenyl)thiophene-2-carboxylate (180 mg, 0.578 mmol) and 2-benzyl-4,4,5,5-tetramethyl-1,3,2-dioxaborolane (164 mg, 0.751 mol) in 1,4-dioxane/H₂O (4 mL, v/v=5:1) was added K₂CO₃ (120 mg, 0.867 mmol) and Pd(dppf)Cl₂ DCM (47.2 mg, 0.0570 mol), the mixture was stirred under nitrogen atmosphere at 80° C. for 16 hours. LCMS showed that the starting material was consumed and ~71% desired product was detected. The mixture was concentrated under reduced pressure to remove the solvent. The crude product was purified by column chromatography (SiO₂, Ethyl acetate/Petroleum ether=0-10%). The desired compound was obtained as yellow oil (140 mg, 68% yield). LCMS (220 nm, 254 nm): t_{R} 1.753 min, purity ≥95%, m/z (ESI): 306.90 [M−H]⁻. ¹H NMR (400 MHz, DMSO-d₆) δ 7.81 (d, J=4.0 Hz, 1H), 7.40-7.12 (m, 9H), 3.95 (s, 2H), 3.84 (s, 3H), 2.35 (s, 3H).

### Synthesis of 5-(5-benzyl-2-methylphenyl)thiophene-2-carboxylic acid (RU-NT-297)

To a solution of methyl 5-(5-benzyl-2-methylphenyl)thiophene-2-carboxylate (140 mg, 0.434 mmol) in THF (1 mL) and MeOH (2 mL) was added NaOH (1.3 mL, 1M), the mixture was stirred at 25° C. for 16 hours. LCMS showed that the starting material was consumed and ~100% desired product was detected. The mixture was adjusted to pH=5 with aq. HCl (5 mL, 1M) and extracted with ethyl acetate (5 mL×3), dried over anhydrous Na₂SO₄, then filtered and concentrated under reduced pressure to remove the solvent. The crude product was purified by prep-HPLC (column: Gemini, Mobile phase: acetonitrile/H₂O (0.1% FA),) to give the desired product as a white solid (90.36 mg, 66% yield). LCMS (220 nm, 254 nm): t_{R} 1.1980 min, purity ≥95%, m/z (ESI): 306.90 [M−H]⁻. HPLC: RT=3.951 min, Area=97.4%. ¹H NMR (400 MHz, DMSO-d₆): δ 13.14 (s, 1H), 7.72 (d, J=3.6 Hz, 1H), 7.33-7.23 (m, 7H), 7.19 (d, J=6.8 Hz, 2H), 3.95 (s, 2H), 2.35 (s, 3H).

### Synthesis of methyl 5-(4-methyl-[ ], 1′-biphenyl]-3-yl)thiophene-2-carboxylate

To a solution of compound methyl 5-(5-bromo-2-methylphenyl)thiophene-2-carboxylate (160 mg, 0.514 mol) and phenylboronic acid (136 mg, 0.668 mol) in 1,4-dioxane/H₂O (4 mL, v/v=5: 1) was added K₂CO₃ (107 mg, 0.771 mmol) and Pd(dppf)Cl₂ DCM (42.0 mg, 0.0510 moll), the mixture was stirred under nitrogen atmosphere at 80° C. for 16 hours. TLC (Petroleum ether: ethyl acetate=10:1, R_{f}(R1)=0.70, R_{f}(P1)=0.50) showed that the starting material was consumed, and a new spot was detected. The mixture was concentrated under reduced pressure to remove the solvent. The crude product was purified by column chromatography (SiO₂, Ethyl acetate/Petroleum ether=0-10%). The desired compound was obtained as white solid (140 mg, 87% yield). LCMS (220 nm, 254 nm): t_{R} 1.770 min, m/z (ESI): N/A. ¹H NMR (400 MHz, DMSO-d₆) δ 7.86 (d, J=4.0 Hz, 1H), 7.70 (t, J=5.2 Hz, 3H), 7.65 (dd, J=8.0, 1.8 Hz, 1H), 7.47 (t, J=7.6 Hz, 3H), 7.41 (d, J=4.0 Hz, 1H), 7.38 (t, J=7.2 Hz, 1H), 3.85 (s, 3H), 2.45 (s, 3H).

### Synthesis of 5-(4-methyl-[1,1′-biphenyl]-3-yl)thiophene-2-carboxylic acid (RU-NT-298)

To a solution of methyl 5-(4-methyl-[1,1′-biphenyl]-3-yl)thiophene-2-carboxylate (140 mg, 0.454 mmol) in THF (1 mL) and MeOH (2 mL) was added NaOH (1.4 mL, 1M), the mixture was stirred at 25° C. for 16 hours. LCMS (ENBJ240941-13-R1) showed that the starting material was consumed and ~100% desired product was detected. The mixture was adjusted to pH=5 with aq. HCl (5 mL, 1M) and extracted with ethyl acetate (5 mL×3), dried over anhydrous Na₂SO₄, then filtered and concentrated under reduced pressure to remove the solvent. The crude product was purified by prep-HPLC (column: Gemini, Mobile phase: acetonitrile/H₂O (0.1% FA)) to give the desired product as a white solid (96.14 mg, 72% yield). LCMS (220 nm, 254 nm): t_{R} 1.285 min, purity ≥95%, m/z (ESI): 292.90 [M−H]⁻. HPLC: RT=3.893 min, Area=99.7%. ¹H NMR (400 MHz, DMSO-d₆) δ 13.16 (s, 1H), 7.77 (d, J=3.6 Hz, 1H), 7.74-7.67 (m, 3H), 7.64 (dd, J=8.0, 2.0 Hz, 1H), 7.53-7.43 (m, 3H), 7.41-7.33 (m, 2H), 2.45 (s, 3H).

### Synthesis of methyl 5-(5-amino-2-methylphenyl)thiophene-2-carboxylate

To a solution of methyl 5-bromothiophene-2-carboxylate (8.00 g, 36.0 mmol) and 4-methyl-3-(4,4,5,5-tetramethyl-1,3,2-dioxaborolan-2-yl)aniline (7.59 g, 32.0 mmol) in 1,4-dioxane/H₂O (120 mL, v/v=5: 1) was added K₂CO₃(7.50 g, 54.0 mmol) and Pd(dppf)Cl₂ DCM (1.77 g, 2.00 mmol), the mixture was stirred under nitrogen atmosphere at 80° C. for 16 hours. LCMS showed that the starting material was consumed and ~86% desired product was detected. Two parallel reactions were set up. The mixture was concentrated under reduced pressure to remove the solvent, then diluted with ethyl acetate (50 mL). The organic phase was washed with water (50 mL×3), dried over anhydrous sodium sulfate and concentrated under reduced pressure to remove the solvent. The crude product was purified by column chromatography (SiO₂, Ethyl acetate/Petroleum ether=0-20%). The desired product was obtained as yellow oil (8.10 g, purity=90% 86% yield). LCMS (220 nm, 254 nm): t_{R} 1.158 min, m/z (ESI): 247.9 [M+H]+. ¹H NMR (400 MHz, DMSO) δ 7.79 (d, J=4.0 Hz, 1H), 7.20 (d, J=4.0 Hz, 1H), 6.98 (d, J=8.0 Hz, 1H), 6.67 (d, J=2.4 Hz, 1H), 6.55 (dd, J=8.0, 2.4 Hz, 1H), 5.07 (s, 2H), 3.83 (s, 3H), 2.22 (s, 3H).

### Synthesis of methyl 5-(5-bromo-2-methylphenyl)thiophene-2-carboxylate

To a solution of methyl 5-(5-amino-2-methylphenyl)thiophene-2-carboxylate (5.40 g, 21.6 mmol) in MeCN (100 mL) and CuBr (6.27 g, 43.6 mmol) was added tert-Butyl nitrite (4.51 g, 43.6 mol) at 0° C. and stirred at 50° C. for 3 hours. TLC (Petroleum ether: ethyl acetate=10:1, R_{f}(R1)=0.70, R_{f}(P1)=0.50) showed that the starting material was consumed, and a new spot was detected. Three parallel reactions were set up. The mixture was filtered through a celite pad. the filtrate diluted with ethyl acetate (50 mL) and concentrated under reduced pressure to remove the solvent. The crude product was purified by column chromatography (SiO₂, Ethyl acetate/Petroleum ether=0-5%). The desired compound was obtained as light-yellow solid (1.68 g, 24.3% yield). LCMS (220 nm, 254 nm): t_{R} 1.365 min, purity ≥95%, m/z (ESI): 310.5 [M+H]+. HPLC: RT=4.397 min, Area=98.0%. ¹H NMR (400 MHz, DMSO-d₆) δ 7.83 (d, J=4.0 Hz, 1H), 7.62 (d, J=2.0 Hz, 1H), 7.54 (dd, J=8.0, 2.0 Hz, 1H), 7.36 (d, J=4.0 Hz, 1H), 7.33 (d, J=8.0 Hz, 1H), 3.85 (s, 3H), 2.36 (s, 3H).

### Synthesis of methyl 5-(5-cyclopropyl-2-methylphenyl)thiophene-2-carboxylate

To a solution of compound methyl 5-(5-bromo-2-methylphenyl)thiophene-2-carboxylate (160 mg, 0.514 mol) and cyclopropylboronic acid (57.4 mg, 0.668 mol) in 1,4-dioxane/H₂O (4 mL, v/v=5: 1) was added K₃PO₄ (218 mg, 1.03 mmol) and Pd(dppf)Cl₂. DCM (42.0 mg, 0.0510 moll, the mixture was stirred under nitrogen atmosphere at 80° C. for 16 hours. TLC (Petroleum ether: ethyl acetate=10: 1, R_{f}(R1)=0.70, R_{f}(P1)=0.50) showed that the starting material was consumed, and a new spot was detected. The mixture was concentrated under reduced pressure to remove the solvent. The crude product was purified by column chromatography (SiO₂, Ethyl acetate/Petroleum ether=0-10%). The desired compound was obtained as a light-yellow oil (110 mg, 73% yield). LCMS (220 nm, 254 nm): t_{R} 1.706 min, purity=93%, m/z (ESI): N/A. ¹H NMR (400 MHz, DMSO-d₆) δ 7.82 (d, J=4.0 Hz, 1H), 7.29 (d, J=4.0 Hz, 1H), 7.22 (d, J=8.0 Hz, 1H), 7.15 (d, J=2.0 Hz, 1H), 7.03 (dd, J=8.0, 2.0 Hz, 1H), 3.84 (s, 3H), 2.34 (s, 3H), 1.98-1.88 (m, 1H), 1.10-0.87 (m, 2H), 0.79-0.58 (m, 2H).

### Synthesis of 5-(5-cyclopropyl-2-methylphenyl)thiophene-2-carboxylic acid (RU-NT-299)

To a solution of methyl 5-(5-cyclopropyl-2-methylphenyl)thiophene-2-carboxylate (110 mg, 0.403 mmol) in THF (1 mL) and MeOH (2 mL) was added NaOH (1.2 mL, 1M), the mixture was stirred at 25° C. for 16 hours. LCMS showed that the starting material was consumed and ~94% desired product was detected. The mixture was adjusted to pH=5 with aq. HCl (5 mL, 1M) and extracted with ethyl acetate (5 mL×3), dried over anhydrous Na₂SO₄, then filtered and concentrated under reduced pressure to remove the solvent. The crude product was purified by prep-HPLC (column: Gemini, Mobile phase: acetonitrile/H₂O (0.1% FA)) to give the desired compound as a white solid (62.42 mg, 58% yield). LCMS (220 nm, 254 nm): t_{R} 1.206 min, purity ≥95%, m/z (ESI): 256.95 [M−H]⁻. HPLC: RT=3.671 min, Area=96.7%. ¹H NMR (400 MHz, DMSO-d₆) δ 13.13 (s, 1H), 7.72 (d, J=3.6 Hz, 1H), 7.25 (d, J=3.6 Hz, 1H), 7.21 (d, J=8.0 Hz, 1H), 7.14 (d, J=1.6 Hz, 1H), 7.01 (dd, J=8.0, 2.0 Hz, 1H), 2.34 (s, 3H), 2.01-1.84 (m, 1H), 1.04-0.88 (m, 2H), 0.76-0.65 (m, 2H).

### Synthesis of 5-(2-bromo-6-methylphenyl)thiophene-2-carboxylic acid (RU-NT-301)

A mixture of methyl 5-(2-bromo-6-methylphenyl)thiophene-2-carboxylate (120 mg, 0.385 mmol) and NaOH (30.8 mg, 0.771 mmol) in THF/H₂O (2 mL, v/v=7: 3) was stirred at 20° C. for 12 hours. LCMS showed that the starting material was consumed completely. The crude product was diluted with water (20 mL) and extracted with MTBE (20 mL). The pH of the aqueous phase was adjusted to 3-4 with aqueous citric acid (~1 mL, 10 wt %), then the aqueous phase was extracted with ethyl acetate (20 mL×3). The organic phase was dried over anhydrous sodium sulfate and concentrated under reduced pressure to remove the solvent. Purification was carried out using preparative HPLC Method B. The solvent for the desired product was concentrated in vacuo to obtain the desired product as a white solid. LCMS (220 nm, 254 nm): t_{R} 1.158 min, purity ≥95%, m/z (ESI): 294.8 [M−H]⁻. HPLC: ENBJ240928-8-P1A, RT=3.733 min, Area=99.8%. ¹H NMR (400 MHz, DMSO-d₆) δ 13.23 (s, 1H), 7.77 (d, J=3.6 Hz, 1H), 7.60 (d, J=8.0 Hz, 1H), 7.37 (d, J=7.6 Hz, 1H), 7.30 (t, J=8.0 Hz, 1H), 7.06 (d, J=4.0 Hz, 1H), 2.16 (s, 3H).

### Synthesis of methyl 5-(2-benzyl-6-methylphenyl)thiophene-2-carboxylate

A mixture of methyl 5-(2-bromo-6-methylphenyl)thiophene-2-carboxylate (150 mg, 0.482 mmol), 2-benzyl-4,4,5,5-tetramethyl-1,3,2-dioxaborolane (136 mg, 0.626 mmol), K₃PO₄ (204 mg, 0.964 mmol) and Pd(dppf)Cl₂ DCM (39.3 mg, 0.0482 mmol) in toluene/H₂O (3.5 mL, v/v=6: 1) was stirred at 80° C. for 12 hours under nitrogen atmosphere. LCMS showed that the starting material was consumed completely. The mixture was concentrated under reduced pressure to remove the solvent, then dissolved with ethyl acetate (20 mL). The organic phase was washed by water (10 mL), dried over anhydrous sodium sulfate and concentrated under reduced pressure to remove the solvent. The crude product was purified by prep-TLC (SiO₂, Petroleum ether/Ethyl acetate=10: 1). The desired product was obtained as yellow oil (185 mg, crude). LCMS (220 nm, 254 nm): t_{R} 1.512 min, m/z (ESI): N/A

### Synthesis of 5-(2-benzyl-6-methylphenyl)thiophene-2-carboxylic acid (RU-NT-302)

To a solution of methyl 5-(2-benzyl-6-methylphenyl)thiophene-2-carboxylate (185 mg, 0.573 mmol) in MeOH/THF (2.5 mL, v/v=3: 2) was added NaOH (45.9 mg, 1.15 mmol) in H₂O (0.5 mL) was stirred at 20° C. for 12 hours. LCMS showed that the starting material was consumed completely. The crude product was diluted with water (20 mL) and extracted with MTBE (20 mL). The pH of the aqueous phase was adjusted to 3-4 with aqueous citric acid (~1 mL, 10 wt %), then the aqueous phase was extracted with ethyl acetate (20 mL×3). The organic phase was dried over anhydrous sodium sulfate and concentrated under reduced pressure to remove the solvent. Purification was carried out using preparative HPLC Method B. The solvent for the desired product was concentrated in vacuo to obtain the desired product as a white solid (31.60 mg, 17% yield). LCMS (220 nm, 254 nm): t_{R} 1.281 min, purity ≥95%, m/z (ESI): 306.9 [M−H]⁻. HPLC: RT=4.360 min, Area=94.8%. ¹H NMR (400 MHz, DMSO-d₆) δ 13.10 (s, 1H), 7.71 (d, J=3.6 Hz, 1H), 7.29 (t, J=7.6 Hz, 1H), 7.25-7.07 (m, 5H), 6.94 (d, J=6.8 Hz, 2H), 6.88 (d, J=3.6 Hz, 1H), 3.80 (s, 2H), 2.09 (s, 3H).

### Synthesis of 5-(2-methyl-6-(phenylethynyl)phenyl)thiophene-2-carboxylic acid (RU-NT-303)

To a solution of methyl 5-(2-methyl-6-(phenylethynyl)phenyl)thiophene-2-carboxylate (130 mg, 0.391 mmol) in MeOH/THF (2.5 mL, v/v=3: 2) was added NaOH (31.2 mg, 0.782 mmol) in H₂O (0.5 mL) and the mixture was stirred at 20° C. for 12 hours. LCMS showed that the starting material was consumed completely. The crude product was diluted with water (20 mL) and extracted with MTBE (20 mL). The pH of the aqueous phase was adjusted to 3-4 with aqueous citric acid (~1 mL, 10 w t %), then the aqueous phase was extracted with ethyl acetate (20 mL×3). The organic phase was dried over anhydrous sodium sulfate and concentrated under reduced pressure to remove the solvent. Purification was carried out using preparative HPLC Method B. The solvent for the desired product was concentrated in vacuo to obtain the desired product as a white solid (57.05 mg, 46% yield). LCMS (220 nm, 254 nm): t_{R} 1.281 min, purity ≥95%, m/z (ESI): 306.9 [M−H]⁻. HPLC: RT=4.360 min, Area=94.8%. ¹H NMR (400 MHz, DMSO-d₆) δ 13.15 (s, 1H), 7.83-7.78 (m, 1H), 7.51 (t, J=4.4 Hz, 1H), 7.44-7.32 (m, 5H), 7.24-7.15 (m, 3H), 2.23 (s, 3H).

### Synthesis of methyl (E)-5-(2-methyl-6-styrylphenyl)thiophene-2-carboxylate

A mixture of methyl 5-(2-bromo-6-methylphenyl)thiophene-2-carboxylate (150 mg, 0.482 mmol), (E)-styrylboronic acid (92.7 mg, 0.626 mmol), K₂CO₃ (99.9 mg, 0.723 mmol) and Pd(dppf)Cl₂ DCM (39.3 mg, 0.0482 mmol) in dioxane/H₂O (3.5 mL, v/v=6: 1) was stirred at 80° C. for 1.5 hours under nitrogen atmosphere. LCMS showed that the starting material was consumed completely. The mixture was concentrated under reduced pressure to remove the solvent, then dissolved with ethyl acetate (20 mL). The organic phase was washed by water (10 mL), dried over anhydrous and concentrated under reduced pressure to remove the solvent. The crude product was purified by prep-TLC (SiO₂, Petroleum ether/Ethyl acetate=10: 1). The desired compound was obtained as yellow gum (130 mg, 66% yield). LCMS (220 nm, 254 nm): t_{R} 1.563 min, purity=82%, m/z (ESI): 335.1 [M+H]+. ¹H NMR (400 MHz, DMSO-d₆) δ 7.91 (d, J=4.0 Hz, 1H), 7.73 (d, J=7.6 Hz, 1H), 7.45-7.09 (m, 9H), 6.82 (d, J=16.4 Hz, 1H), 3.84 (s, 3H), 2.14 (s, 3H).

### Synthesis of (E)-5-(2-methyl-6-styrylphenyl)thiophene-2-carboxylic acid (RU-NT-304)

A mixture of methyl (E)-5-(2-methyl-6-styrylphenyl)thiophene-2-carboxylate (130 mg, 0.388 mmol) and NaOH (31.0 mg, 0.777 mmol) in MeOH/H₂O (3 mL, v/v=2: 1) was stirred at 20° C. for 12 hours. LCMS showed that the starting material was consumed completely. The crude product was diluted with water (20 mL) and extracted with MTBE (20 mL). The pH of the aqueous phase was adjusted to 3-4 with aqueous citric acid (~1 mL, 10 wt %), then the aqueous phase was extracted with ethyl acetate (20 mL×3). The organic phase was dried over anhydrous sodium sulfate and concentrated under reduced pressure to remove the solvent. Purification was carried out using preparative HPLC Method B. The solvent for the desired product was concentrated in vacuo to obtain the desired product as a white solid (76.12 mg, 60.5% yield). LCMS (220 nm, 254 nm): t_{R} 1.246 min, purity ≥95%, m/z (ESI): 310.0 [M−H]⁻. HPLC: RT=4.078 min, Area=99.0% H NMR (400 MHz, DMSO-d₆) δ 13.19 (s, 1H), 7.81 (d, J=3.6 Hz, 1H), 7.73 (d, J=7.6 Hz, 1H), 7.40 (t, J=7.6 Hz, 1H), 7.36-7.21 (m, 6H), 7.18 (d, J=16.4 Hz, 1H), 7.07 (d, J=3.6 Hz, 1H), 6.84 (d, J=16.4 Hz, 1H), 2.14 (s, 3H).

### Synthesis of methyl 5-(2-methyl-6-phenethylphenyl)thiophene-2-carboxylate

A mixture of methyl 5-(2-methyl-6-(phenylethynyl)phenyl)thiophene-2-carboxylate (150 mg, 0.451 mmol) and Pd/C (42.0 mg, 0.0451 mmol) in MeOH/THF (3 mL, v/v=2: 1) was stirred at 20° C. for 1.5 hours under H2 (15 psi) atmosphere. LCMS showed that the starting material was consumed completely. The mixture was filtered and concentrated under reduced pressure to remove the solvent. The crude product was used for the next step without any purification. The desired compound was obtained as yellow oil (153 mg, 98% yield). LCMS (220 nm, 254 nm): t_{R} 1.560 min, purity ≥95%, m/z (ESI): 337 [M+H]⁺. HPLC: RT=4.555 min, Area=99.9%. ¹H NMR (400 MHz, DMSO-d₆) δ 7.86 (d, J=3.6 Hz, 1H), 7.33-7.27 (m, 1H), 7.26-7.11 (m, 5H), 7.04-6.97 (m, 3H), 3.84 (s, 3H), 2.82-2.58 (m, 4H), 2.08 (s, 3H).

### Synthesis of 5-(2-methyl-6-phenethylphenyl)thiophene-2-carboxylic acid (RU-NT-305)

To a mixture of methyl 5-(2-methyl-6-phenethylphenyl)thiophene-2-carboxylate (153 mg, 0.454 mmol) in MeOH/THF (2.5 mL, v/v=3: 2) was added NaOH (36.3 mg, 0.909 mmol) in H₂O (0.5 mL) and the mixture was stirred at 20° C. for 12 hours. LCMS showed that the starting material was consumed completely. The crude product was diluted with water (20 mL) and extracted with MTBE (20 mL). The pH of the aqueous phase was adjusted to 3-4 with aqueous citric acid (~1 mL, 10 wt %), then the aqueous phase was extracted with ethyl acetate (20 mL×3). The organic phase was dried over anhydrous sodium sulfate and concentrated under reduced pressure to remove the solvent Purification was carried out using preparative HPLC Method B. The solvent for the desired product was concentrated in vacuo to obtain the desired product as a white solid (87.74 mg, 60% yield). LCMS (220 nm, 254 nm): t_{R} 1.343 min, purity ≥95%, m/z (ESI): 321 [M−H]⁻. HPLC: RT=4.555 min, Area=99.9%. ¹H NMR (400 MHz, DMSO-d₆) δ 13.14 (s, 1H), 7.77 (d, J=4.0 Hz, 1H), 7.29 (t, J=7.6 Hz, 1H), 7.25-7.11 (m, 5H), 7.03-6.95 (m, 3H), 2.79-2.61 (m, 4H), 2.09 (s, 3H).

### Synthesis of 3-methyl-2-(4,4,5,5-tetramethyl-1,3,2-dioxaborolan-2-yl)aniline

A mixture of 2-bromo-3-methylaniline (24.0 g, 129 mmol), Pin₂B₂ (42.5 g, 167 mmol), KOAc (25.3 g, 258 mmol) and Pd(dppf)Cl₂ (6.61 g, 9.03 mmol) in dioxane (480 mL) was stirred at 80° C. for 12 hours under nitrogen atmosphere. LCMS showed that the starting material was consumed completely. No work-up. The desired compound (30.0 g, crude, in dioxane) was used for the next step without any purification. LCMS (220 nm, 254 nm): t_{R} 0.943 min, purity=80%, m/z (ESI): 234.1 [M+H]⁺.

### Synthesis of methyl 5-(2-amino-6-methylphenyl)thiophene-2-carboxylate

A mixture of 3-methyl-2-(4,4,5,5-tetramethyl-1,3,2-dioxaborolan-2-yl)aniline (30.0 g, 84.4 mmol), methyl 5-(2-amino-5-methylphenyl)thiophene-2-carboxylate (16.0 g, 72.3 mmol), K₂CO₃ (15.0 g, 108 mmol) and Pd(dppf)Cl₂ (2.65 g, 3.62 mmol) in dioxane/H₂O (540 mL, v/v=6:1) was stirred at 80° C. for 8 hours under nitrogen atmosphere. LCMS showed that the starting material was consumed and ~26% desired product was detected. The mixture was concentrated under reduced pressure to remove the solvent, then diluted with ethyl acetate (500 mL). The organic phase was washed with water (200 mL×3), dried over anhydrous sodium sulfate and concentrated under reduced pressure to remove the solvent. The crude product was purified by column chromatography (SiO₂, Ethyl acetate/Petroleum ether=0-15%). The desired product was obtained as yellow oil (8.54 g, 61% yield). LCMS (220 nm, 254 nm): t_{R} 0.984 min, purity=80%, m/z (ESI): 247.6 [M+H]⁺. ¹H NMR (400 MHz, DMSO-d₆) δ 7.86 (d, J=3.6 Hz, 1H), 7.03-6.96 (m, 2H), 6.59 (d, J=8.0 Hz, 1H), 6.49 (d, J=7.2 Hz, 1H), 4.74 (s, 2H), 2.00 (s, 3H).

### Synthesis of methyl 5-(2-amino-5-methylphenyl)thiophene-2-carboxylate

To a mixture of methyl 5-bromothiophene-2-carboxylate (700 mg, 3.17 mmol), 4-methyl-2-(4,4,5,5-tetramethyl-1,3,2-dioxaborolan-2-yl)aniline (849 mg, 3.64 mmol) and K₂CO₃ (874 mg, 6.33 mmol) in 1,4-dioxane/H₂O (24 mL, v/v=5: 1) was added Pd(dppf)Cl₂ (232 mg, 0.317 mmol) under N₂ and heated to 80° C. Then the reaction mixture was stirred at 80° C. for 12 hours. The mixture was diluted with water (200 mL) and extracted with dichloromethane (100 mL×3). The combined organic phase was dried over anhydrous sodium sulfate and concentrated. The residue was purified by flash column chromatography (SiO₂, Ethyl acetate/Petroleum ether=0-4%) to obtain the desired compound as a white solid (700 mg, 89.4% yield). LCMS (220 nm, 254 nm): t_{R} 1.025 min, purity ≥95%, m/z (ESI): 248.0 [M+H]⁺. ¹H NMR (400 MHz, DMSO-d₆) δ 7.79 (d, J=4.0 Hz, 1H), 7.35 (d, J=4.0 Hz, 1H), 7.07 (s, 1H), 6.93 (dd, J=8.0, 1.6 Hz, 1H), 6.74 (d, J=8.0 Hz, 1H), 5.04 (s, 2H), 3.83 (s, 3H), 2.18 (s, 3H).

### Synthesis of methyl 5-(2-cyclopropyl-5-methylphenyl)thiophene-2-carboxylate

To a mixture of methyl 5-(2-amino-5-methylphenyl)thiophene-2-carboxylate (600 mg, 2.43 mmol) and CuBr (696 mg, 4.85 mmol) in MeCN (10 mL) was added tBuONO (500 mg, 4.85 mmol) in MeCN (5 mL) dropwise at 0° C. and the mixture was stirred at 50° C. for 12 hours. The mixture was filtered through a Celite pad, and the filtrate was concentrated to give crude product. The crude product was purified by flash column chromatography (SiO₂, Ethyl acetate/Petroleum ether=0-2%) to obtain the desired compound as a yellow solid (260 mg, 33% yield). LCMS (220 nm, 254 nm): t_{R} 1.415 min, purity=97%, m/z (ESI): 310.9 [M+H]⁺. ¹H NMR (400 MHz, DMSO-d₆) δ 7.83 (d, J=4.0 Hz, 1H), 7.66 (d, J=8.0 Hz, 1H), 7.48-7.44 (m, 1H), 7.41 (d, J=4.0 Hz, 1H), 7.20 (dd, J=8.0, 1.6 Hz, 1H), 3.85 (s, 3H), 2.32 (s, 3H).

### Synthesis of 5-(2-cyclopropyl-5-methylphenyl)thiophene-2-carboxylic acid (RU-NT-306)

To a solution of methyl 5-(2-cyclopropyl-5-methylphenyl)thiophene-2-carboxylate (240 mg, 0.881 mmol) in MeOH/H₂O (5 mL, v/v=4: 1) was added sodium hydroxide (70.5 mg, 1.76 mmol) and then the reaction was stirred at 20° C. for 12 hours. The reaction was quenched by citric acid (50 mL, 2%) and extracted with Ethyl acetate (50 mL×3). The organic phase was washed with brine water (50 mL×3), then dried over anhydrous sodium sulfate, filtered and concentrated to remove the solvent. Purification was carried out using preparative HPLC Method B. The solvent for the desired product was concentrated in vacuo to obtain the desired product as a white solid (130 mg, 57% yield). LCMS (220 nm, 254 nm): t_{R} 1.229 min, purity ≥95%, m/z (ESI): 259.0 [M+H]+. HPLC: RT=4.196 min, Area=97%. HPLC: RT=3.707 min, Area=99.789%. ¹H NMR (400 MHz, DMSO-d₆) δ 13.09 (s, 1H), 7.73 (d, J=3.6 Hz, 1H), 7.31 (d, J=3.6 Hz, 1H), 7.28-7.22 (m, 1H), 7.15 (d, J=8.0 Hz, 1H), 6.99 (d, J=8.0 Hz, 1H), 2.30 (s, 3H), 2.05-1.92 (m, 1H), 0.98-0.82 (m, 2H), 0.77-0.56 (m, 2H).

### Synthesis of methyl 5-(2-bromo-6-methylphenyl)thiophene-2-carboxylate (RU-NT-307)

To a mixture of methyl 5-(2-amino-6-methylphenyl)thiophene-2-carboxylate (6.54 g, 26.4 mmol) and CuBr (7.59 g, 52.8 mmol) in MeCN (120 mL) was added tBuONO (5.45 g, 52.8 mmol) in MeCN (30 mL) dropwise at 0° C. and the mixture was stirred at 50° C. for 12 hours. LCMS showed that the starting material was consumed completely. The mixture was cooled to room temperature and added to aq. NH₃·H₂O (500 mL, 2 wt %). The mixture was extracted with ethyl acetate (200 mL×3). The combined organic phase was washed by saturated aq. NaCl (200 mL×3), dried over anhydrous sodium sulfate and concentrated under reduced pressure to remove the solvent. The crude product was purified by column chromatography (SiO₂, Ethyl acetate/Petroleum ether=0-3%). The desired compound was obtained as yellow oil (2.02 g, 30% yield). LCMS (220 nm, 254 nm): t_{R} 1.381 min, purity ≥95%, m/z (ESI): 310.9 [M+H]⁺. HPLC: RT=4.196 min, Area=97%. ¹H NMR (400 MHz, DMSO-d₆) δ 7.86 (d, J=4.0 Hz, 1H), 7.60 (d, J=7.6 Hz, 1H), 7.37 (d, J=7.6 Hz, 1H), 7.30 (t, J=7.6 Hz, 1H), 7.10 (d, J=3.6 Hz, 1H), 3.84 (s, 3H), 2.15 (s, 3H).

### Synthesis of methyl 2-amino-3-cyclopropylbenzoate

To a stirred mixture of methyl 2-amino-3-bromobenzoate (1.00 g, 4.30 mmol), cyclopropylboronic acid (0.44 g, 5.16 mmol) and K₂CO₃ (1.19 g, 8.60 mmol) in dioxane/H₂O (v/v=4/1, 20 mL) was added Pd(dppf)Cl₂ DCM (0.35 g, 0.43 mmol) under N₂ and heated to 80° C. for 12 hours. LCMS showed that the starting material was consumed, and the desired product was detected as a major peak. The mixture was diluted with water (10 mL) and extracted with ethyl acetate (20 mL×3). The organic phase was concentrated under reduced pressure. The crude product was purified by column chromatography (SiO₂, Ethyl acetate: Petroleum ether=0-5%) to give the desired compound as a yellow oil (750 mg, 86% yield). LCMS (220 nm, 254 nm): t_{R} 1.150 min, purity ≥95%, m/z (ESI): 192.1 [M+H]⁺. ¹H NMR (400 MHz, DMSO-d₆) δ 7.62 (dd, J=8.0, 1.2 Hz, 1H), 7.10 (d, J=7.2 Hz, 1H), 6.61 (s, 2H), 6.52-6.46 (m, 1H), 3.79 (s, 3H), 1.73-1.59 (m, 1H), 0.96-0.84 (m, 2H), 0.55-0.44 (m, 2H)

### Synthesis of methyl 2-bromo-3-cyclopropylbenzoate

To a solution of methyl 2-amino-3-cyclopropylbenzoate (750 mg, 3.92 mmol) in MeCN (10 mL) was added CuBr (1.12 g, 7.84 mmol) and tert-Butyl nitrite (809 mg, 7.84 mmol) at 0° C. Then the reaction mixture was heated to 40° C. for 12 hours. TLC (Petroleum ether:Ethyl acetate=3:1, R_{f}(R1)=0.20, R_{f}(P1)=0.40) showed that the starting material was consumed, and a new spot was detected. The mixture was filtered, and the filtrate was concentrated under reduced pressure. The crude product was purified by column chromatography (SiO₂, Ethyl acetate: Petroleum ether=0-15%) to give compound the desired compound (600 mg, 57% yield) as a yellow oil. ¹H NMR (400 MHz, DMSO-d₆) δ 7.42 (dd, J=7.6, 1.6 Hz, 1H), 7.37 (t, J=7.6 Hz, 1H), 7.19 (dd, J=7.6, 1.6 Hz, 1H), 3.86 (s, 3H), 2.21-2.11 (m, 1H), 1.06-0.99 (m, 2H), 0.75-0.67 (m, 2H).

### Synthesis of 2-bromo-3-cyclopropylbenzoic acid

To a solution of methyl 2-bromo-3-cyclopropylbenzoate (600 mg, 3.14 mmol) in MeOH (10 mL) was added a solution of LiOH (150 mg, 6.27 mmol) in H₂O (2 mL). Then the reaction mixture was stirred at 20° C. for 2 hours. TLC (Petroleum ether:Ethyl acetate=3:1, R_{f}(R1)=0.60, R_{f}(P1)=0.10) showed that starting material was consumed, and a new spot was detected. Adjusted pH of the mixture to 3~4 with aq. citric acid (10 mL, 5%) and extracted with DCM (10 mL×3). The organic phase was concentrated under reduced pressure. The crude product was purified by column chromatography (SiO₂, Ethyl acetate: Petroleum ether=0-30%) to give the desired compound as a white solid (400 mg, 68% yield). ¹H NMR (400 MHz, DMSO-d₆) δ 13.33 (s, 1H), 7.39 (dd, J=7.6, 1.6 Hz, 1H), 7.34 (t, J=7.6 Hz, 1H), 7.14 (dd, J=7.6, 1.6 Hz, 1H), 2.22-2.09 (m, 1H), 1.07-0.97 (m, 2H), 0.76-0.65 (m, 2H)

### Synthesis of 2-bromo-3-cyclopropyl-N-methoxy-N-methylbenzamide

To a mixture of 2-bromo-3-cyclopropylbenzoic acid (400 mg, 2.26 mmol), N,O-dimethylhydroxylamine (264 mg, 2.71 mmol) and DIEA (1.17 g, 9.03 mmol) in DMF (8 mL) was added HATU (1.03 g, 2.71 mmol) at 0° C. Then the reaction mixture was stirred at 20° C. for 1 hour. LCMS showed that the starting material was consumed, and the desired product was detected as a major peak. The mixture was diluted with saturated aq. NH₄Cl (20 mL) and extracted with ethyl acetate (20 mL×3). The organic phase was concentrated under reduced pressure. The crude product was purified by column chromatography (SiO₂, Ethyl acetate: Petroleum ether=0-10%) to give compound 7 (350 mg, 52% yield) as a colorless oil. LCMS (220 nm, 254 nm): t_{R} 1.016 min, purity ≥95%, m/z (ESI): 283.9 [M+H]+. ¹H NMR (400 MHz, DMSO-d₆) δ 7.33 (t, J=7.6 Hz, 1H), 7.18 (dd, J=7.6, 1.6 Hz, 1H), 7.04 (d, J=7.6 Hz, 1H), 3.86-3.38 (m, 3H), 3.30-2.92 (m, 3H), 2.19-2.09 (m, 1H), 1.08-0.96 (m, 2H), 0.77-0.65 (m, 2H).

### Synthesis of benzo[b]thiophen-2-yl(2-bromo-3-cyclopropylphenyl)methanone

To a solution of benzo[b]thiophene (141.69 mg, 1.055 mmol) in THF (5 mL) was added n-BuLi (0.5 mL, 1.27 mmol) under N₂ at −78° C. Then the reaction mixture was stirred at −78° C. for 1 hour. Then a solution of 2-bromo-3-cyclopropyl-N-methoxy-N-methylbenzamide (300 mg, 1.055 mmol) in THF (5 mL) was added to the reaction mixture at −78° C. and stirred at −78° c. for 1 hour. LCMS showed that ~17% of start material was consumed, and ~80% of desired product was detected. The mixture was diluted with saturated aq. NH₄Cl (20 mL) and extracted with ethyl acetate (20 mL×3). The organic phase was concentrated under reduced pressure. The crude product was purified by column chromatography (SiO₂, Ethyl acetate: Petroleum ether=0-8%) to give the desired product as a yellow oil (200 mg, 50% yield). LCMS (220 nm, 254 nm): t_{R} 1.463 min, purity ≥95%, m/z (ESI): 356.9 [M+H]+. ¹H NMR (400 MHz, DMSO-d₆) δ 8.11 (d, J=8.4 Hz, 1H), 8.04 (d, J=8.0 Hz, 1H), 7.80 (s, 1H), 7.61-7.54 (m, 1H), 7.50-7.43 (m, 2H), 7.40 (dd, J=7.6, 1.6 Hz, 1H), 7.23 (dd, J=7.6, 1.6 Hz, 1H), 2.25-2.15 (m, 1H), 1.11-1.03 (m, 2H), 0.83-0.75 (m, 2H).

### Synthesis of Methyl5-(2-(benzo[b]thiophene-2-carbonyl)-6-cyclopropylphenyl)thiophene-2-carboxylate

To a mixture of benzo[b]thiophen-2-yl(2-bromo-3-cyclopropylphenyl)methanone (200 mg, 0.56 mmol), (5-(methoxycarbonyl)thiophen-2-yl)boronic acid (125 mg, 0.67 mmol) and K₂CO₃ (155 mg, 1.12 mmol) in dioxane/H₂O (v/v=4/1, 5 mL) was added Pd(dtbpf)Cl₂ (36.49 mg, 0.056 mmol) under N₂. Then the reaction mixture was heated to 80° C. and stirred for 12 hours. LCMS showed that the starting material was consumed, and the desired product was detected as a major peak. The mixture was diluted with saturated aq. NH₄Cl (10 mL) and extracted with ethyl acetate (20 mL×3). The organic phase was concentrated under reduced pressure. The crude product was purified by column chromatography (SiO₂, Ethyl acetate: Petroleum ether=0-10%) to give the desired product (140 mg, 57% yield) as a yellow oil. LCMS (220 nm, 254 nm): t_{R} 1.457 min, purity ≥95%, m/z (ESI): 419.1 [M+H]⁺. ¹H NMR (400 MHz, DMSO-d₆) δ 8.03 (t, J=8.8 Hz, 2H), 7.84 (s, 1H), 7.66 (d, J=3.6 Hz, 1H), 7.58-7.51 (m, 2H), 7.46 (dd, J=14.0, 6.8 Hz, 2H), 7.27 (d, J=7.6 Hz, 1H), 7.05 (d, J=3.6 Hz, 1H), 3.75 (s, 3H), 1.89-1.76 (m, 1H), 0.94-0.84 (m, 2H), 0.82-0.74 (m, 2H).

### Synthesis of 5-(2-(benzo[b]thiophene-2-carbonyl)-6-cyclopropylphenyl)thiophene-2-carboxylic acid (RU-NT-308)

According to the general procedure for saponification of methyl esters. Methyl 5-(2-(benzo[b]thiophene-2-carbonyl)-6-cyclopropylphenyl)thiophene-2-carboxylate (140 mg, 0.334 mmol), MeOH (3 mL), LiOH (2 eq), H₂O (1 mL). LCMS showed that the starting material was consumed, and the desired product was detected as a major peak. Adjusted pH of the mixture to 5~6 with aq. HCl (1 mL, 1M). Purification was carried out using preparative HPLC Method B. The solvent for the desired product was concentrated in vacuo to give the desired product as a white solid (90.0 mg, 63.2% yield). LCMS (220 nm, 254 nm): t_{R} 1.206 min, purity ≥95%, m/z (ESI): 405.0 [M+H]⁺. HPLC: RT=3.896 min, Area=99.86%. H NMR (400 MHz, DMSO-d₆) δ 13.10 (s, 1H), 8.03 (t, J=8.4 Hz, 2H), 7.83 (s, 1H), 7.58-7.51 (m, 3H), 7.49-7.41 (m, 2H), 7.25 (dd, J=8.0, 1.0 Hz, 1H), 7.00 (d, J=3.6 Hz, 1H), 1.91-1.78 (m, 1H), 0.95-0.85 (m, 2H), 0.83-0.75 (m, 2H).

### Synthesis of 5-(2-ethylphenyl)-1,3,4-thiadiazole-2-carboxylic acid (RU-NT-313)

To a solution of methyl 5-bromo-1,3,4-thiadiazole-2-carboxylate (0.11 g, 0.5 mmol) in dioxane (4 mL) was added (2-ethylphenyl)boronic acid (3.35 g, 22.34 mmol) and 2M Na₂CO₃ (2 mL). This mixture was degassed with a stream of argon for 2 min.

Tetrakis(triphenylphosphine)palladium (29 mg, 5 mol %) was added and this mixture was heated at reflux overnight under argon. After cooling to room temperature, the mixture was filtered through cotton to remove Pd impurities, the solvent was concentrated under vacuo and the sample purification was carried out using preparative HPLC Method A. The product was lyophilized to give an off-white solid (19 mg, 16.1%). LCMS (220 nm, 254 nm): t_{R} 2.360 min, purity ≥95%, m/z (ESI): 235 [M+H]⁺. ¹H NMR (500 MHz, CDCl₃) δ 9.19 (s, 1H), 7.58 (dt, J=7.8, 1.7 Hz, 1H), 7.44 (t, J=7.5 Hz, 1H), 7.39 (d, J=7.8 Hz, 1H), 7.31 (dd, J=7.5, 1.8 Hz, 1H), 2.92 (q, 7.6 Hz, 2H), 1.21 (t, J=7.6 Hz, 3H). (WO2011112828)

### Synthesis of methyl 2-(2-ethylphenyl)-1,3-thiazole-5-carboxylate

According to the general procedure for Suzuki coupling reactions using Method A, precatalyst CL. Methyl 2-bromo-1,3-thiazole-5-carboxylate (0.1 g, 1.0 eq), 2-ethylphenyl)boronic acid (81.05 mg, 0.54 mmol), dry dioxane (4 mL). Temperature: 130° C. Purification was carried out using preparative HPLC Method A. White solid (30 mg, 27% yield). LCMS (220 nm, 254 nm): t_{R} 3.247 min, purity ≥95%, m/z (ESI): 248.10 [M+H]⁺. Synthesis of 2-(2-ethylphenyl)-1,3-thiazole-5-carboxylic acid (RU-NT-318) According to the general procedure for saponification of methyl esters. Methyl 2-(2-ethylphenyl)-1,3-thiazole-5-carboxylate (10 mg, 0.04 mmol). Purification was carried out using preparative HPLC Method B. The solvent for the desired product was concentrated in vacuo and the sample was lyophilized O/N. White solid (5 mg, 53% yield). LCMS (220 nm, 254 nm): t_{R} 2.584 min, purity ≥95%, m/z (ESI): 234.15 [M+H]⁺. ¹H NMR (500 MHz, CD₃OD) δ 8.31 (s, 1H), 7.58 (dd, J=7.7, 1.4 Hz, 1H), 7.44 (td, J=7.5, 1.4 Hz, 1H), 7.39 (d, J=7.7 Hz, 1H), 7.31 (td, J=7.5, 1.5 Hz, 1H), 2.93 (q, J=7.5 Hz, 2H), 1.16 (t, J=7.5 Hz, 3H).

### Synthesis of methyl 5-(2-ethylphenyl)-1,3-thiazole-2-carboxylate

According to the general procedure for Suzuki coupling reactions using Method A, precatalyst CL. Methyl 5-bromo-1,3-thiazole-2-carboxylate (0.15 g, 1 eq), 2-ethylphenyl)boronic acid (81.05 mg, 0.54 mmol), dry dioxane (4 mL). Temperature: 130° C. White solid (30 mg, 27% yield). LCMS (220 nm, 254 nm): t_{R} 2.951 min, purity ≥95%, m/z (ESI): 248.10 [M+H]⁺.

### Synthesis of 5-(2-ethylphenyl)-1,3-thiazole-2-carboxylic acid (RU-NT-319)

According to the general procedure for saponification of methyl esters. Methyl 5-(2-ethylphenyl)-1,3-thiazole-2-carboxylate (30 mg, 0.32 mmol). Purification was carried out using preparative HPLC Method A. The solvent for the desired product was concentrated in vacuo and the sample was lyophilized. The desired product was obtained as a white solid (30.0 mg, 40% yield). LCMS (220 nm, 254 nm): t_{R} 2.427 min, purity ≥95%, m/z (ESI): 234.15 [M+H]⁺. ¹H NMR (500 MHz, CD₃OD) δ 7.75 (s, 1H), 7.40-7.31 (m, 2H), 7.25 (ddd, J=7.2, 5.7, 2.7 Hz, 1H), 2.73 (q, J=7.6 Hz, 2H), 1.16 (t, J=7.6 Hz, 2H).

### Synthesis of methyl 5-(2-formyl-6-methylphenyl)thiophene-2-carboxylate

In a 100 ml three-neck flask, [5-(methoxycarbonyl)thiophen-2-yl]boronic acid (0.75 g, 4.03 mmol), 2-bromo-3-methylbenzaldehyde (1.04 g, 5.24 mmol), tetrakis(triphenylphosphane) palladium (9 mol %) and Na₂CO₃ (0.85 g, 8.06 mmol) were mixed with toluene (15 mL), ethanol (5 mL) and water (5 mL) under nitrogen atmosphere. The resulting mixture was heated to 80° C. overnight. After completion of the reaction, the organic layer was extracted with toluene 2-3 times and brine. The organic layers were combined and dried over anhydrous MgSO₄, followed by purification by silica gel column chromatography 80 g column normal phase 0-10% gradient EtOAc in Hex. This afforded a white solid which was further purified fraction via wash with 0.5 mL MeOH and 0.5 mL ACN (residue, 0.4 g, 36% yield). LCMS (220 nm, 254 nm): t_{R} 2.951 min, purity ≥95%, m/z (ESI): 247.10 [M+H]⁺. ¹H NMR (500 MHz, DMSO-d₆) δ 10.08 (d, J=0.8 Hz, 1H), 7.95 (dd, J=8.3, 1.6 Hz, 1H), 7.86 (d, J=3.8 Hz, 1H), 7.79 (td, J=8.3, 1.2 Hz, 1H), 7.66 (td, J=7.6 Hz, 1H), 7.38 (d, J=3.8 Hz, 1H), 3.86 (s, 3H).

### Synthesis of 5-(2-formyl-6-methylphenyl)thiophene-2-carboxylic acid (RU-NT-320)

According to the general procedure for saponification of methyl esters. Methyl 5-(2-formyl-6-methylphenyl)thiophene-2-carboxylate (26 mg, 0.08 mmol). Purification was carried out using preparative HPLC Method B. Sample was concentrated under vacuo and lyophilized O/N to afford the tile compound as a white solid (10 mg, 53% yield). LCMS (220 nm, 254 nm): t_{R} 2.541 min, purity ≥95%, m/z (ESI): 248.30 [M+H]⁺. ¹H NMR (300 MHz, CD₃OD) δ 7.85 (dt, J=2.8, 1.4 Hz, 1H), 7.72-7.58 (m, 1H), 7.60-7.34 (m, 2H), 6.99 (dt, J=3.8, 1.3 Hz, 1H), 6.71 (m, 1H), 2.22 (s, 3H).

### Synthesis of 5-{2-[(]Z)-2-cyanoeth-1-en-1-yl]-6-methylphenyl}thiophene-2-carboxylic acid & 5-{2-[(]E)-2-cyanoeth-1-en-1-yl]-6-methylphenyl}thiophene-2-carboxylic acid (RU-NT-321)

According to the general procedure for saponification of methyl esters. Cis and trans mixture of methyl 5-{2-[(1Z)-2-cyanoeth-1-en-1-yl]-6-methylphenyl}thiophene-2-carboxylate (36 mg, 0.13 mmol). Purification was carried out using preparative HPLC Method B. Sample was concentrated under vacuo and lyophilized O/N to afford the tile compound as a white solid for the cis and trans mixture (trans:cis 3:1, 20.0 mg, 49% yield). LCMS (220 nm, 254 nm): t_{R} 2.584 min, purity ≥95%, m/z (ESI): 308.65 [M+K]⁺. ¹H NMR (500 MHz, DMSO-d₆) δ 7.80 (dd, J=3.7, 1.0 Hz, 2H), 7.77 (dd, J=3.7, 1.1 Hz, 1H), 7.70 (dd, J=6.3, 3.0 Hz, 1H), 7.56-7.38 (m, 4H), 7.24-7.10 (m, 2H), 7.11-6.96 (m, 2H), 6.37 (d, J=16.6 Hz, 1H), 5.82 (d, J=12.0 Hz, OH), 2.17 (s, 3H), 2.13 (s, 3H).

### Synthesis of 5-(2,6-diethylphenyl)thiophene-2-carbonitrile

In a 20 mL scintillation vial with a septum was charged with (5-cyanothiophen-2-yl)boronic acid (1.0 eq), Pd(dppf)Cl₂.CH₂Cl₂ (0.1 mol %), and 2-bromo-1,3-diethylbenzene (0.4 g, 1 eq) in 1:1 (v/v) Toluene/MeOH (1 mL) and was purged with N₂ under vigorous stirring, and 2.0 M aqueous K₂CO₃ solution (2.0 eq) was added slowly. The mixture was heated at 90° C. O/N. After cooling, Purification was carried out using preparative HPLC Method B to afford the title product as a yellow oil (40 mg, 10% yield). LCMS (220 nm, 254 nm): t_{R} 3.707 min, purity ≥95%, m/z (ESI): 282 [M+CH₃CN]⁺. ¹H NMR (500 MHz, CDCl₃) 7.62-7.66 (m, 1H), 7.35 (t, J=7.6 Hz, 1H), 7.16 (d, J=7.7 Hz, 2H), 6.88 (dd, J=3.7, 0.6 Hz, 1H), 2.41 (q, J=7.6 Hz, 4H), 1.10 (td, J=7.6, 0.6 Hz, 6H).

### Synthesis of 5-[5-(2,6-diethylphenyl)thiophen-2-yl]-1H-1,2,3,4-tetrazole (RU-NT-323)

According to the general procedure for tetrazole formation. 5-(2,6-Diethylphenyl)thiophene-2-carbonitrile (30 mg, 0.12 mmol). Purification was carried out using preparative HPLC Method B. Solvent was concentrated under vacuo and lyophilized O/N to afford the tile compound as a White solid (17 mg, 48% yield). LCMS (220 nm, 254 nm): t_{R} 3.048 min, purity ≥95%, m/z (ESI): 316.25 [M+CH₃CN]⁺. ¹H NMR (500 MHz, (CD₃)₂CO) δ 7.90 (d, J=3.7 Hz, 1H), 7.36 (dd, J=8.1, 7.3 Hz, 1H), 7.22 (d, J=7.6 Hz, 2H), 7.08 (d, J=3.7 Hz, 1H), 2.50 (q, J=7.6 Hz, 4H), 1.11 (t, J=7.5 Hz, 6H).

### Synthesis of (E)-5-(2,6-diethylphenyl)-N′-hydroxythiophene-2-carboximidamide (RU-NT-325) & 5-(2,6-diethylphenyl)thiophene-2-carboxamide (RU-NT-324)

In a 20 mL scintillation vial containing 5-(2,6-diethylphenyl)thiophene-2-carbonitrile (30 mg, 0.12 mmol) in ethanol (1 mL) was added hydroxylamine hydrochloride (6.16 mg, 0.19 mmol) and TEA (18.87 mg, 0.19 mmol), The reaction mixture was stirred at 80° C. overnight. Solvent was concentrated in vacuo. Purification was carried out using preparative HPLC Method B. The fraction collected was concentrated in vacuo before being placed on the V-10 for complete dryness. The desired hydroxylamidine analogue was obtained as a white solid (7.3 mg, 21% yield) and the amide analogue side product was obtained as a white solid (1.3 mg, 4% yield). (E)-5-(2,6-diethylphenyl)-N-hydroxythiophene-2-carboximidamide: LCMS (220 nm, 254 nm): t_{R} 2.330 min, purity ≥95%, m/z (ESI): 275.20 [M+CH₃CN]⁺.¹H NMR (500 MHz, Acetone-d₆) δ 7.58 (d, J=3.6 Hz, 1H), 7.42-7.23 (m, 1H), 7.18 (d, J=7.6 Hz, 2H), 6.90 (d, J=3.6 Hz, 1H), 2.46 (q, J=7.6 Hz, 4H), 1.08 (t, J=7.6 Hz, 6H). 5-(2,6-Diethylphenyl)thiophene-2-carboxamide: LCMS (220 nm, 254 nm): t_{R} 2.828 min, purity ≥95%, m/z (ESI): 301.20 [M+CH₃CN]₊. ¹H NMR (500 MHz, Acetone-d₆) δ 7.76 (d, J=3.7 Hz, 1H), 7.31 (d, J=7.3 Hz, 1H), 7.18 (d, J=7.6 Hz, 2H), 6.92 (d, J=3.7 Hz, 1H), 2.45 (q, J=7.5 Hz, 4H), 1.08 (t, J=7.5 Hz, 6H).

### Synthesis of 3-[5-(2,6-diethylphenyl)thiophen-2-yl]-4,5-dihydro-1,2,4-oxadiazol-5-one (RU-NT-326)

In a 20 mL scintillation vial was added N,N′-carbonyldiimidazole (4.26 mg, 0.03 mmol) and 1,8-diazabicyclo[5.4.0]undec-7-ene (9 μL, 0.02 mmol) to (E)-5-(2,6-diethylphenyl)-N-hydroxythiophene-2-carboximidamide (6.0 mg, 0.02 mmol). Add 1,4-dioxane (1.0 mL) to the system. Stir the reaction mixture at 100° C. for 3 hours. Cool the mixture to ambient temperature and dilute the mixture with 2M HCl until the pH was adjusted to ~2. Purification was carried out using preparative HPLC Method B. Solvent was concentrated in vacuo and further dryness was carried out using the V-10. The desired product was obtained as a white solid (5.6 mg, 85.3% yield). LCMS (220 nm, 254 nm): t_{R} 3.188 min, purity ≥95%, m/z (ESI): 302.40 [M+H]⁺. ¹H NMR (500 MHz, Acetone-d₆) δ 7.83 (d, J=3.7 Hz, 1H), 7.36 (dd, J=8.0, 7.3 Hz, 1H), 7.21 (d, J=7.6 Hz, 2H), 7.08 (d, J=3.7 Hz, 1H), 2.47 (q, J=7.6 Hz, 4H), 1.10 (t, J=7.6 Hz, 6H).

### Synthesis of 5-(2,6-diethylphenyl)-N-methanesulfonylthiophene-2-carboxamide (RU-NT-327)

To a solution of 5-(2,6-diethylphenyl)thiophene-2-carboxylic acid (100 mg, 0.38 mmol) in CH₂CI₂ (1 mL) was added EDCI (119.25 mg, 0.77 mmol), DMAP (93.85 mg, 0.77 mmol) and methanesulfonamide (36.54 mg, 0.38 mmol). After stirring for 12 h at rt, the solution was diluted with CH₂CI₂ (5 mL) and washed with 2 M HCl until a pH of 2 and brine. Extraction was carried out three times using EtOAc and brine. The organic layers were combined and dried over anhydrous MgSO₄ and the solvent was concentrated under reduced pressure. Purification was carried out using preparative HPLC Method B. Solvent was concentrated in vacuo and further dryness was carried out using the V-10 to afford the desired product as a white crystals (39 mg, 31% yield). LCMS (220 nm, 254 nm): t_{R} 3.067 min, purity ≥95%, m/z (ESI): 338.45 [M+H]⁺. ¹H NMR (500 MHz, Acetone-d₆) δ 10.70 (br s, 1H), 8.09 (d, J=3.8 Hz, 1H), 7.56-7.29 (m, 1H), 7.20 (d, J=7.7 Hz, 2H), 7.03 (dd, J=3.8, 0.6 Hz, 1H), 3.41 (s, 3H), 2.44 (q, J=7.5 Hz, 4H), 1.09 (t, J=7.5 Hz, 6H).

### Synthesis of methyl 5-[2-(cyclopent-1-en-1-yl)-6-methylphenyl]thiophene-2-carboxylate

According to the general procedure for Suzuki coupling using Method B. K₂CO₃ (66.62 mg, 3.0 eq), Pd(PPh₃)₄(9.28 mg, 5 mol %), methyl 5-(2-bromo-6-methylphenyl)thiophene-2-carboxylate (60 mg, 1.0 eq) and cyclopent-1-en-1-ylboronic acid (0.19 mmol, 1.2 eq) in a toluene/ethanol/H₂O (3/1/1) mixture (3.3 mL). Reflux, 110° C. in under N₂ overnight. Purification was carried out using preparative HPLC Method B. Colorless oil (47.1 mg, 98% yield). LCMS (220 nm, 254 nm): t_{R} 3.912 min, purity ≥95%, m/z (ESI): 340.20 [M+CH₃CN]+. ¹H NMR (500 MHz, CDCl₃) δ 7.78 (d, J=3.8 Hz, 1H), 7.28 (d, J=7.6 Hz, 1H), 7.20 (t, J=6.9 Hz, 1H), 6.87 (d, J=3.7 Hz, 1H), 5.56 (p, J=2.2 Hz, 1H), 3.93 (s, 3H), 2.33 (tq, J=7.3, 2.5 Hz, 2H), 2.22 (s, 3H), 1.88-1.72 (m, 2H), 1.53-1.19 (m, 1H).

### Synthesis of 5-[2-(cyclopent-1-en-1-yl)-6-methylphenyl]thiophene-2-carboxylic acid (RU-NT-328)

According to the general procedure for saponification of methyl esters. Methyl 5-[2-(cyclopent-1-en-1-yl)-6-methylphenyl]thiophene-2-carboxylate (36.1 mg, 0.12 mmol). Purification was carried out using preparative HPLC Method B. Solvent was concentrated under vacuo and lyophilized O/N to afford the tile compound as a white solid (21 mg, 61% yield). LCMS (220 nm, 254 nm): t_{R} 3.273 min, purity ≥95%, m/z (ESI): 326.15 [M+CH₃CN]+. ¹H NMR (500 MHz, Acetone-d₆) δ 7.63 (d, J=3.7 Hz, 1H), 7.14 (t, J=7.6 Hz, 1H), 7.11-7.01 (m, 2H), 6.83 (d, J=3.7 Hz, 1H), 5.45-5.38 (m, 1H), 2.14 (ddt, J=10.0, 4.8, 2.4 Hz, 2H), 2.09-2.06 (m, 2H), 2.05 (s, 3H), 1.90 (p, J=2.2 Hz, 1H), 1.72-1.45 (m, 2H).

### Synthesis of 3-methyl-2-(4,4,5,5-tetramethyl-1,3,2-dioxaborolan-2-yl)aniline

A mixture of 2-bromo-3-methylaniline (24.0 g, 129 mmol), Pin₂B₂ (42.5 g, 167 mmol), KOAc (25.3 g, 258 mmol) and Pd(dppf)Cl₂ (6.61 g, 9.03 mmol) in dioxane (480 mL) was stirred at 80° C. for 12 hours under nitrogen atmosphere. LCMS showed that the starting material was consumed completely. No work-up. The titled compound (30.0 g, crude, in dioxane) was used for the next step without any purification.

### Synthesis of methyl 5-(2-amino-6-methylphenyl)thiophene-2-carboxylate

A mixture of 3-methyl-2-(4,4,5,5-tetramethyl-1,3,2-dioxaborolan-2-yl)aniline (30.0 g, 84.4 mmol), methyl 5-bromothiophene-2-carboxylate (16.0 g, 72.3 mmol), K₂CO₃ (15.0 g, 108 mmol) and Pd(dppf)Cl₂ (2.65 g, 3.62 mmol) in dioxane/H₂O (540 mL, v/v=6: 1) was stirred at 80° C. for 8 hours under nitrogen atmosphere. LCMS showed that the starting material was consumed and ~26% desired product was detected. The mixture was concentrated under reduced pressure to remove the solvent, then diluted with ethyl acetate (500 mL). The organic phase was washed with water (200 mL×3), dried over anhydrous sodium sulfate and concentrated under reduced pressure to remove the solvent. The crude product was purified by column chromatography (SiO₂, Ethyl acetate/Petroleum ether=0-15%). Titled compound (8.54 g, 79.6 wt % purity, 61% yield) was obtained as yellow oil. LCMS (220 nm, 254 nm): t_{R} 0.984 min, purity ≥95%, m/z (ESI): 247.6 [M+H]⁺. ¹H NMR (400 MHz, DMSO-d₆) δ 7.86 (d, J=3.6 Hz, 1H), 7.03-6.96 (m, 2H), 6.59 (d, J=8.0 Hz, 1H), 6.49 (d, J=7.2 Hz, 1H), 4.74 (s, 2H), 2.00 (s, 3H).

### Synthesis of methyl 5-(2-bromo-6-methylphenyl)thiophene-2-carboxylate

To a mixture of methyl 5-(2-amino-6-methylphenyl)thiophene-2-carboxylate (6.54 g, 26.4 mmol) and CuBr (7.59 g, 52.8 mmol) in MeCN (120 mL) was added tBuONO (5.45 g, 52.8 mmol) in MeCN (30 mL) dropwise at 0° C. and the mixture was stirred at 50° C. for 12 hours. LCMS showed that the starting material was consumed completely. The mixture was cooled to room temperature and added to aq. NH₃·H₂O (500 mL, 2 wt %). The mixture was extracted with ethyl acetate (200 mL×3). The combined organic phase was washed by saturated aq. NaCl (200 mL×3), dried over anhydrous sodium sulfate and concentrated under reduced pressure to remove the solvent. The crude product was purified by column chromatography (SiO₂, Ethyl acetate/Petroleum ether=0-3%), (2.02 g, 97.2% purity, 30.0% yield) was obtained as yellow oil. LCMS (220 nm, 254 nm): t_{R} 1.381 min, purity ≥95%, m/z (ESI): 310.9 [M+H]⁺. ¹H NMR (400 MHz, DMSO-d₆) δ 7.86 (d, J=4.0 Hz, 1H), 7.60 (d, J=7.6 Hz, 1H), 7.37 (d, J=7.6 Hz, 1H), 7.30 (t, J=7.6 Hz, 1H), 7.10 (d, J=3.6 Hz, 1H), 3.84 (s, 3H), 2.15 (s, 3H).

### Synthesis of 4,4,5,5-tetramethyl-2-[(JE)-2-(5-methylthiophen-2-yl)ethenyl]-1,3,2-dioxaborolane

In a 250 mL RBF, a mixture of vinylboronate pinacol ester (0.96 g, 1.1 eq), 2-bromo-5-methylthiophene (1.0 g, 1.0 eq), DIPEA (1.97 mL, 2.0 eq), Pd₂(dba)₃ (0.28 mmol, 5 mol %) and P(t-Bu)₃HBF₄ (0.56 mmol, 10 mol %) in dry toluene (10 mL) was stirred at 95° C. for 3 hours under a N₂ atmosphere. The reaction mixture was concentrated under vacuum. Water was added to the residual mixture and extraction was carried out three times with EtOAc. The organic layer was combined and dried over anhydrous MgSO₄ and filtered. The solvent was evaporated under vacuum. Purification was carried out by silica gel chromatography using a 24 g size column with EtOAc/n-hexene as the eluent to obtain the desired product as a pink solid (0.46 g, 32% yield). LCMS (220 nm, 254 nm): t_{R} 3.511 min, purity ≥95%, m/z (ESI): 251.15 [M+H]⁺. ¹H NMR (300 MHz, CDCl₃) δ 7.38 (dd, J=18.0, 1.3 Hz, 1H), 6.86 (d, J=3.5 Hz, 1H), 6.66-6.59 (m, 1H), 5.76 (d, J=18.0 Hz, 1H), 2.45 (s, 3H), 1.29 (s, 12H).

### Synthesis of methyl 5-{2-methyl-6-[(]Z)-2-(5-methylthiophen-2-yl)ethenyl]phenyl}thiophene-2-carboxylate & methyl 5-{2-methyl-6-[(]E)-2-(5-methylthiophen-2-yl)ethenyl]phenyl}thiophene-2-carboxylate

According to the general procedure for Suzuki coupling reactions using Method A, precatalyst C1. 4,4,5,5-Tetramethyl-2-[(1E)-2-(5-methylthiophen-2-yl)ethenyl]-1,3,2-dioxaborolane (57.88 mg, 0.23 mmol), 4,4,5,5-tetramethyl-2-[(1E)-2-(5-methylthiophen-2-yl)ethenyl]-1,3,2-dioxaborolane (18 mg, 0.02 mmol), dry dioxane (6 mL). Temperature: 130° C. Purification gradient 60-70%. (Trans: white solid, 14 mg, 22% yield, cis: 4 mg, off-white solid taken to the next step without further purification), trans product-LCMS (220 nm, 254 nm): t_{R} 3.889 min, purity ≥95%, m/z (ESI): 355.10 [M+H]⁺, cis isomer-LCMS (220 nm, 254 nm): t_{R} 3.797 min, purity ≥95%, m/z (ESI): 355.10 [M+H]⁺. ¹H NMR for the trans isomer (500 MHz, CDCl₃) δ 7.86 (d, J=3.7 Hz, 1H), 7.53 (d, J=7.9 Hz, 1H), 7.30 (t, J=7.7 Hz, 1H), 7.17 (d, J=7.5 Hz, 1H), 7.02 (d, J=15.9 Hz, 1H), 6.89 (d, J=3.7 Hz, 1H), 6.77 (d, J=3.5 Hz, 1H), 6.66-6.46 (m, 1H), 3.92 (s, 3H), 2.42 (s, 3H), 2.18 (s, 3H).

### Synthesis of 5-{2-methyl-6-[(]E)-2-(5-methylthiophen-2-yl)ethenyl]phenyl}thiophene-2-carboxylic acid (RU-NT-330)

According to the general procedure for saponification of methyl esters. (Methyl 5-{2-methyl-6-[(1E)-2-(5-methylthiophen-2-yl)ethenyl]phenyl}thiophene-2-carboxylate (14 mg, 0.04 mmol). Purification was carried out using preparative HPLC Method B. Solvent was concentrated under vacuo and lyophilized O/N to afford the tile compound as a white solid (5 mg, 37% yield). LCMS (220 nm, 254 nm): t_{R} 3.413 min, purity ≥95%, m/z (ESI): 341 [M+H]⁺. ¹H NMR (500 MHz, MeOD-d₄) δ 7.85 (d, J=3.7 Hz, 1H), 7.59 (d, J=7.9 Hz, 1H), 7.31 (t, J=7.7 Hz, 1H), 7.19 (d, J=7.5 Hz, 1H), 7.12 (d, J=16.0 Hz, 1H), 6.93 (d, J=3.7 Hz, 1H), 6.78 (d, J=3.5 Hz, 1H), 6.66-6.59 (m, 1H), 6.53 (d, J=16.0 Hz, 1H), 2.39 (s, 3H), 2.17 (s, 3H).

### Synthesis of 5-{2-methyl-6-[(]Z)-2-(5-methylthiophen-2-yl)ethenyl]phenyl}thiophene-2-carboxylic acid (RU-NT-331)

According to the general procedure for saponification of methyl esters. (Methyl 5-{2-methyl-6-[(1Z)-2-(5-methylthiophen-2-yl)ethenyl]phenyl}thiophene-2-carboxylate (4 mg, 0.01 mmol). Purification was carried out using preparative HPLC Method B. Solvent was concentrated under vacuo and lyophilized O/N to afford the tile compound as a white solid (3.84 mg, 78% yield). LCMS (220 nm, 254 nm): t_{R} 3.361 min, purity ≥95%, m/z (ESI): 341.10 [M+H]⁺. ¹H NMR (500 MHz, MeOD-d₄) δ 7.75 (s, 1H), 7.56 (d, J=3.7 Hz, 1H), 7.37-7.27 (m, 2H), 7.23-7.14 (m, 2H), 6.69 (d, J=3.8 Hz, 1H), 6.59-6.44 (m, 1H), 6.31 (d, J=3.5 Hz, 1H), 2.38 (d, J=1.1 Hz, 3H), 2.22 (s, 3H).

### Synthesis of methyl 5-[2-(cyanomethyl)-6-methylphenyl]thiophene-2-carboxylate

According to the general procedure for Suzuki coupling reactions using Method A, precatalyst C1. [5-(Methoxycarbonyl)thiophen-2-yl]boronic acid (265 mg, 1.43 mmol), 2-(2-bromo-3-methylphenyl)acetonitrile (300 mg, 1.43 eq), dry dioxane (7 mL). Temperature: 130° C. The crude mixture (78.1 mg, yellow oil) was taken to the next step without further purification. LCMS (220 nm, 254 nm): t_{R} 2.892 min, m/z (ESI): 272.10 [M+H]⁺.

### Synthesis of methyl 5-{2-methyl-6-[(H-1,2,3,4-tetrazol-5-yl)methyl]phenyl}thiophene-2-carboxylate

According to general procedure for tetrazole formation. Methyl 5-[2-(cyanomethyl)-6-methylphenyl]thiophene-2-carboxylate (71 mg, 0.26 mmol). Temperature: 120° C. The crude mixture was taken to the next step without any further purification. LCMS (220 nm, 254 nm): t_{R} 2.900 min, m/z (EI): 314.25 [M]⁺.

### Synthesis of 5-{2-methyl-6-[(1H-1,2,3,4-tetrazol-5-yl)methyl]phenyl}thiophene-2-carboxylic acid (RU-NT-350)

According to the general procedure for saponification of methyl esters. (Methyl 5-{2-methyl-6-[(1H-1,2,3,4-tetrazol-5-yl)methyl]phenyl}thiophene-2-carboxylate](12 mg, 0.04 mmol). Purification was carried out using preparative HPLC Method B. Solvent was concentrated under vacuo and lyophilized O/N to afford the tile compound as a white solid (5.90 mg, 52% yield). LCMS (220 nm, 254 nm): t_{R} 1.945 min, purity ≥95%, m/z (ESI): 342.10 [M+CH₃CN]⁺. ¹H NMR (500 MHz, DMSO-d₆) δ 13.15 (s, 1H), 7.71 (d, J=3.7 Hz, 1H), 7.36 (t, J=7.6 Hz, 1H), 7.29 (d, J=7.5 Hz, 1H), 7.20 (dd, J=7.7, 1.4 Hz, 1H), 6.87 (d, J=3.7 Hz, 1H), 4.09 (s, 2H), 2.10 (s, 3H).

### Synthesis of N-(2-bromo-3-methylphenyl)-1-benzothiophene-2-carboxamide

According to general procedure A for amide formation. 2-Bromo-3-methylaniline (0.3 g, 1.61 mmol), commercially available 1-benzothiophene-2-carbonyl chloride (0.35 g, 1.77mol), DCM (3 mL). Yellow crystals (210 mg, 38% yield). LCMS (220 nm, 254 nm): t_{R} 3.340 min, purity ≥95%, m/z (ESI): 348.10 [M+H]⁺.

### Synthesis of methyl 5-[2-(]-benzothiophene-2-amido)-6-methylphenyl]thiophene-2-carboxylate

According to the general procedure for Suzuki coupling reactions using Method A, precatalyst C2. N-(2-bromo-3-methylphenyl)-1-benzothiophene-2-carboxamide (0.2 g, 0.58 mmol), [5-(methoxycarbonyl)thiophen-2-yl]boronic acid (0.12 g, 0.64 mmol), dry dioxane (5 mL). Temperature: 100° C. Brownish solid (97.7 mg, 41% yield). LCMS (220 nm, 254 nm): t_{R} 3.251 min, m/z (ESI): 408.25 [M+H]⁺. ¹H NMR (500 MHz, DMSO-d₆) δ 10.05 (s, 1H), 8.01 (d, J=7.5 Hz, 1H), 7.97-7.90 (m, 1H), 7.78 (d, J=3.8 Hz, 1H), 7.52-7.39 (m, 4H), 7.34 (dd, J=8.0, 4.4 Hz, 2H), 7.13 (d, J=3.8 Hz, 1H), 3.77 (s, 3H), 2.22 (s, 3H).

### Synthesis of 5-[2-(-benzothiophene-2-amido)-6-methylphenyl]thiophene-2-carboxylic acid (RU-NT-358)

According to the general procedure for saponification of methyl esters. Methyl 5-[2-(1-benzothiophene-2-amido)-6-methylphenyl]thiophene-2-carboxylate (15 mg, 0.02 mmol). Purification was carried out using preparative HPLC Method B. Solvent was concentrated under vacuo and lyophilized O/N to afford the tile compound as a white solid (4 mg, 43% yield). LCMS (220 nm, 254 nm): t_{R} 2.781 min, purity ≥95%, m/z (ESI): 394.29 [M+H]⁺. ¹H NMR (500 MHz, DMSO-d₆) δ 13.05 (s, 1H), 10.00 (s, 1H), 8.10-7.97 (m, 2H), 7.97-7.85 (m, 1H), 7.69 (d, J=3.8 Hz, 1H), 7.55-7.39 (m, 3H), 7.34 (t, J=8.3 Hz, 2H), 7.09 (d, J=3.8 Hz, 1H), 2.23 (s, 3H).

### Synthesis of methyl 5-[2-methyl-6-(N-methyl-]-benzothiophene-2-amido)phenyl]thiophene-2-carboxylate

According to the general procedure for N-alkylation of amides (Method A). Methyl-5-[2-(1-benzothiophene-2-amido)-6-methylphenyl]thiophene-2-carboxylate (75 mg, 1 eq), K₂CO₃ (11.1 mg, 0.06 mmol), DMF (1 mL). Temperature: 70° C. for 4 hours. The crude mixture was taken directly to the next step. LCMS (220 nm, 254 nm): t_{R} 3.285 min, m/z (ESI): 422.20 [M+H]⁺.

### Synthesis of 5-[2-methyl-6-(N-methyl-1-benzothiophene-2-amido)phenyl]thiophene-2-carboxylic acid (RU-NT-360)

According to the general procedure for saponification of methyl esters. Crude material from the previous step was taken for the saponification without purification. Purification was carried out using preparative HPLC Method B. Solvent was concentrated under vacuo and lyophilized O/N to afford the tile compound as a white solid (10 mg, 90% yield). LCMS (220 nm, 254 nm): t_{R} 2.845 min, purity ≥95%, m/z (ESI): 408.20 [M+H]⁺. ¹H NMR (500 MHz, DMSO-d₆) δ 12.76 (s, 1H), 7.89 (d, J=8.0 Hz, 1H), 7.74 (d, J=7.9 Hz, 1H), 7.69-7.52 (m, 2H), 7.44-7.25 (m, 2H), 7.03 (s, 1H), 6.86 (d, J=3.8 Hz, 1H), 3.31-2.99 (m, 3H), 2.19 (s, 3H).

### Synthesis of N-(2-bromo-3-methylphenyl)-6-methyl-1-benzothiophene-2-carboxamide

According to general procedure A for amide formation. 2-Bromo-3-methylaniline (0.26 g, 1.4 mmol), commercially available 6-methyl-1-benzothiophene-2-carbonyl chloride (0.26 g, 1.28 mmol), THF (10 mL). Rtp, O/N. Yellow crystals (0.1 g, 22% yield). LCMS (220 nm, 254 nm): t_{R} 3.553 min, purity ≥95%, m/z (ESI): 362.10 [M+H]⁺. ¹H NMR (500 MHz, DMSO-d₆) δ 10.25 (s, 1H), 8.28 (s, 1H), 7.89 (d, J=8.2 Hz, 1H), 7.85 (s, 1H), 7.39 (dd, J=7.7, 2.0 Hz, 1H), 7.34 (d, J=7.5 Hz, 1H), 7.32-7.26 (m, 2H), 2.46 (s, 3H), 2.43 (s, 3H).

### Synthesis of methyl 5-[2-methyl-6-(6-methyl-1-benzothiophene-2-amido)phenyl]thiophene-2-carboxylate

According to the general procedure for Suzuki coupling reactions using Method A, precatalyst C2. N-(2-Bromo-3-methylphenyl)-6-methyl-1-benzothiophene-2-carboxamid (200 mg, 0.58 mmol), [5-(methoxycarbonyl)thiophen-2-yl]boronic acid (0.12 g, 0.64 mmol), dry dioxane (5 mL). Temperature: 100° C. Brown solid (90.5 mg, 37% yield). LCMS (220 nm, 254 nm): t_{R} 3.441 min, purity ≥95%, m/z (ESI): 422.20 [M+H]⁺. ¹H NMR (500 MHz, DMSO-d₆) δ 9.96 (s, 1H), 7.93 (s, 1H), 7.87-7.68 (m, 1H), 7.42 (t, J=7.7 Hz, 1H), 7.39-7.27 (m, 2H), 7.26 (dd, J=8.2, 1.6 Hz, 1H), 7.12 (d, J=3.8 Hz, 1H), 3.77 (s, 3H), 2.43 (s, 3H), 2.22 (s, 3H).

### Synthesis of 5-[2-methyl-6-(6-methyl-1-benzothiophene-2-amido)phenyl]thiophene-2-carboxylic acid (RU-NT-359)

According to the general procedure for saponification of methyl esters. Methyl 5-[2-methyl-6-(6-methyl-1-benzothiophene-2-amido)phenyl]thiophene-2-carboxylate (10 mg, 0.02 mmol). Purification was carried out using preparative HPLC Method B. Solvent was concentrated under vacuo and lyophilized O/N to afford the tile compound as an off-white solid (6.4 mg, 66% yield). LCMS (220 nm, 254 nm): t_{R} 2.936 min, purity ≥95%, m/z (ESI): 408.25 [M+H]⁺. ¹H NMR (500 MHz, DMSO-d₆) δ 13.05 (s, 1H), 9.92 (s, 1H), 7.92 (s, 1H), 7.82-7.76 (m, 2H), 7.41 (t, J=7.7 Hz, 1H), 7.33 (dd, J=11.1, 7.5 Hz, 2H), 7.26 (dd, J=8.3, 1.4 Hz, 1H), 7.08 (d, J=3.7 Hz, 1H), 2.43 (s, 3H), 2.22 (s, 3H).

### Synthesis of methyl 5-[2-methyl-6-(N-methyl-6-methyl-]-benzothiophene-2-amido)phenyl]thiophene-2-carboxylate

According to the general procedure for N-alkylation of amides (Method A). Methyl-5-[2-methyl-6-(6-methyl-1-benzothiophene-2-amido)phenyl]thiophene-2-carboxylate (75 mg, 1 eq), K₂CO₃ (11.1 mg, 0.06 mmol), DMF (1 mL). Rtp, O/N. The reaction was then heated at 70° C. for 4 hours. The crude mixture was taken directly to the next step. LCMS (220 nm, 254 nm): t_{R} 3.285 min, purity ≥95%, m/z (ESI): 422.20 [M+H]⁺.

### Synthesis of 5-[2-methyl-6-(N-methyl-6-methyl-1-benzothiophene-2-amido)phenyl]thiophene-2-carboxylic acid (RU-NT-361)

According to the general procedure for saponification of methyl esters. Crude material from the previous step. Purification was carried out using preparative HPLC Method B. Solvent was concentrated under vacuo and lyophilized O/N to afford the tile compound as a white solid (9 mg, 93% yield). LCMS (220 nm, 254 nm): t_{R} 3.011 min, purity ≥95%, m/z (ESI): 422.25 [M+H]⁺. ¹H NMR (500 MHz, DMSO-d₆) δ 13.16 (s, 1H), 7.82-7.56 (m, 4H), 7.54-7.31 (m, 3H), 7.17 (d, J=8.3 Hz, 1H), 6.93 (s, 1H), 6.84 (d, J=3.8 Hz, 1H), 6.53 (s, 1H), 3.11 (s, 3H), 2.38 (s, 4H), 2.19 (s, 3H).

### Synthesis of (Z)-but-2-en-2-ylboronic acid

To a solution of but-2-yne (2.00 g, 37.0 mmol) in DCM (10 mL) was added BHBr2 Me₂S (37 mL, 37.0 mmol) at −10° C. dropwise over 15 minutes. Then the reaction mixture was stirred at 20° C. for 1 hour. TLC (Petroleum ether: ethyl acetate=10:1, R_{f}(R1)=0.50, R_{f}(P1)=0.30) showed that the starting material was consumed, and a new spot was detected. The mixture was diluted with saturated aq. NH₄Cl (20 mL) and extracted with DCM (20 mL×3). The organic phase was concentrated under reduced pressure. The crude product was purified by column chromatography (SiO₂, ethyl acetate: Petroleum ether=0-5%) to give the title compound (1.70 g, 43.8% yield) as a white solid. ¹H NMR: (400 MHz, CDCl₃) δ 6.92-6.77 (m, 1H), 1.81-1.77 (m, 3H), 1.76 (s, 3H).

### Synthesis of methyl (E)-2-amino-3-(but-2-en-2-yl)benzoate

To a mixture of methyl 2-amino-3-bromobenzoate (3.00 g, 13.0 mmol), (1.56 g, 15. (Z)-but-2-en-2-ylboronic acid (6 mmol) and K₂CO₃ (3.59 g, 26.0 mmol) in dioxane/H₂O (v/v=4/1, 30 mL) was added Pd(dppf)Cl₂DCM (1.06 g, 1.30 mmol) under N₂. Then the reaction mixture was heated to 80° C. and stirred at 80° C. for 12 hours. LCMS showed that starting material was consumed, and the desired product was detected as a major peak. The mixture was diluted with saturated aq. NH₄Cl (100 mL) and extracted with ethyl acetate (100 mL×3). The organic phase was concentrated under reduced pressure. The crude product was purified by column chromatography (SiO₂, ethyl acetate: Petroleum ether=0-10%) to give the title compound (2.30 g, 81.5% yield) as a colorless oil. LCMS (220 nm, 254 nm): t_{R} 1.258 min, purity ≥95%, m/z (ESI): 206.1 [M+H]⁺. ¹H NMR: (400 MHz, DMSO-d₆) δ 7.65 (dd, J=8.0, 1.6 Hz, 1H), 7.05 (dd, J=7.2, 1.6 Hz, 1H), 6.60-6.45 (m, 1H), 6.26 (s, 2H), 5.45 (qd, J=6.8, 1.6 Hz, 1H), 3.79 (s, 3H), 1.88-1.82 (m, 3H), 1.74 (dd, J=6.8, 0.8 Hz, 3H).

### Synthesis of methyl 2-amino-3-(sec-butyl)benzoate

To a solution of methyl (E)-2-amino-3-(but-2-en-2-yl)benzoate (2.00 g, 9.70 mmol) in MeOH (60 mL) was added Pd/C (0.21 g, 1.94 mmol) and stirred under H2 (15 Psi) at 20° C. for 2 hours. LCMS showed that starting material was consumed, and the desired product was detected as a major peak. The mixture was filtered, and the filtrate was concentrated under reduced pressure. The crude product was purified by column chromatography (SiO₂, ethyl acetate: Petroleum ether=0-5%) to give the title compound (1.81 mg, 89.7% yield) as a colorless oil. The product was confirmed by LCMS, HPLC and ¹H NMR. LCMS (220 nm, 254 nm): t_{R} 1.310 min, purity ≥95%, m/z (ESI): 207.6 [M+H]⁺. HPLC: RT=3.866 min, Area=99.86%. ¹H NMR (400 MHz, DMSO-d₆) δ 7.63 (dd, J=8.0, 1.6 Hz, 1H), 7.21 (dd, J=7.6, 1.6 Hz, 1H), 6.64 (s, 2H), 6.56 (t, J=7.6 Hz, 1H), 3.79 (s, 3H), 2.93-2.74 (m, 1H), 1.68-1.41 (m, 2H), 1.13 (d, J=6.8 Hz, 3H), 0.83 (t, J=7.6 Hz, 3H).

### Synthesis of methyl 2-amino-3-(butan-2-yl)benzoate

To a solution of methyl (E)-2-amino-3-(but-2-en-2-yl)benzoate (2.00 g, 9.70 mmol) in MeOH (60 mL) was added Pd/C (0.21 g, 1.94 mmol) and stirred under H2 (15 Psi) at 20° C. for 2 hours. LCMS showed that starting material was consumed, and the desired product was detected as a major peak. The mixture was filtered, and the filtrate was concentrated under reduced pressure. The crude product was purified by column chromatography (SiO₂, ethyl acetate: Petroleum ether=0-5%) to give the title compound (1.81 mg, 89.7% yield) as a colorless oil. LCMS (220 nm, 254 nm): t_{R} 1.310 min, purity ≥95%, m/z (ESI): 207.6 [M+H]⁺. HPLC: RT=3.866 min, Area=99.86%. ¹H NMR (400 MHz, DMSO-d₆) δ 7.63 (dd, J=8.0, 1.6 Hz, 1H), 7.21 (dd, J=7.6, 1.6 Hz, 1H), 6.64 (s, 2H), 6.56 (t, J=7.6 Hz, 1H), 3.79 (s, 3H), 2.93-2.74 (m, 1H), 1.68-1.41 (m, 2H), 1.13 (d, J=6.8 Hz, 3H), 0.83 (t, J=7.6 Hz, 3H).

### Synthesis of methyl 2-bromo-3-(butan-2-yl)benzoate

To a solution of methyl 2-amino-3-(butan-2-yl)benzoate (2.2 g, 18.7 mmol) in MeCN (53 mL) in a 250 mL RBF was added CuBr (4.74 g, 21.23 mmol) and tert-Butyl nitrite (2.19 g, 21.23 mmol) at 0° C. Then the reaction mixture was heated to 70° C. for 3 days. The mixture was filtered, and the filtrate was concentrated under reduced pressure. Purification was carried out using amino silica (28 g size column) using 0-5% EtOAc in hexane gradient to give the desired product with 67% purity as a reddish oil (1.8 g, 67% yield). LCMS (220 nm, 254 nm): t_{R} 3.343 min, purity ≥95%, m/z (ESI): 271.05 [M+H]⁺.

### Synthesis of 2-bromo-3-(butan-2-yl)-N-methoxy-N-methylbenzamide

To a mixture of 2-bromo-3-(butan-2-yl)benzoic acid (1.6 g, 6.2 mmol), methoxy(methyl)amine (0.73 g, 7.47 mmol) and DIEA (1.61 g, 12.45 mmol) in DMF (15 mL) was added HATU (2.84 g, 7.47 mmol) at 0° C. Then the reaction mixture was stirred at 20° C. for 1 hour. The mixture was diluted with saturated aq. NH₄Cl (100 mL) and extracted with ethyl acetate (100 mL×3). The organic phase was concentrated under reduced pressure. The crude product was purified by column chromatography (SiO₂, Ethyl acetate: Petroleum ether=0-10%) to give 2-bromo-3-(butan-2-yl)-N-methoxy-N-methylbenzamide (1.6 g, 86% yield) as a purple oil (87% purity). LCMS (220 nm, 254 nm): t_{R} 2.800 min, purity ≥95%, m/z (ESI): 301.95 [M+H]⁺. ¹H NMR (500 MHz, CDCl₃) δ 7.30 (t, J=7.5 Hz, 1H), 7.23 (dd, J=7.8, 1.7 Hz, 1H), 7.13-7.03 (m, 1H), 3.45 (s, 3H), 3.37 (s, 3H), 3.23 (q, J=7.0 Hz, 1H), 1.77-1.41 (m, 2H), 1.20 (d, J=6.9 Hz, 3H), 0.86 (t, J=7.4 Hz, 3H).

### Synthesis of [2-bromo-3-(butan-2-yl)phenyl](thiophen-2-yl)methanone

To a solution of 2-bromo-3-(butan-2-yl)-N-methoxy-N-methylbenzamide (33.3 mg, 0.11 mmol) in dry THF (0.5 mL) was added 2-lithiothiophene (9.91 mg, 0.11 mmol) under N₂ at −78° C. Then the reaction mixture was stirred at −78° C. for 1 hour. The mixture was diluted with saturated aq. NH₄Cl (20 mL) and extracted with ethyl acetate (20 mL×3). The organic phase was concentrated under reduced pressure and the crude was taken directly to the next step without further purification. LCMS (220 nm, 254 nm): t_{R} 3.487 min, purity ≥95%, m/z (ESI): 323.10 [M+H]⁺.

### Synthesis of methyl 5-[2-(butan-2-yl)-6-(thiophene-2-carbonyl)phenyl]thiophene-2-carboxylate

According to the general procedure for Suzuki coupling reactions using Method A, precatalyst CL. Methyl [2-bromo-3-(butan-2-yl)phenyl](thiophen-2-yl)methanone (100 mg, 0.31 mmol), [5-(methoxycarbonyl)thiophen-2-yl]boronic acid (57 mg, 0.31 mmol), dry dioxane (5 mL). Temperature: 100° C. Brown solid (100 mg, 84% yield). LCMS (220 nm, 254 nm): t_{R} 3.540 min, purity ≥95%, m/z (ESI): 385.30 [M+H]⁺. ¹H NMR (500 MHz, DMSO-d₆) δ 8.05 (dd, J=4.9, 1.2 Hz, 1H), 7.66 (d, J=3.8 Hz, 1H), 7.66-7.55 (m, 2H), 7.50-7.33 (m, 2H), 7.18 (dd, J=4.9, 3.8 Hz, 1H), 6.90 (d, J=3.8 Hz, 1H), 3.78 (s, 3H).

### Synthesis of 5-[2-(butan-2-yl)-6-(thiophene-2-carbonyl)phenyl]thiophene-2-carboxylic acid (RU-NT-362)

According to the general procedure for saponification of methyl esters. Crude mixture (21.6 mg, 0.06 mmol). Purification was carried out using preparative HPLC Method B. Solvent 5 was concentrated under vacuo and lyophilized O/N to afford the tile compound as a white solid (4.7 mg, 23% yield). LCMS (220 nm, 254 nm): t_{R} 3.060 min, purity ≥95%, m/z (ESI): 371.15 [M+H]⁺.

¹H NMR (500 MHz, DMSO-d₆) δ 13.19 (s, 1H), 8.04 (dd, J=4.9, 1.2 Hz, 1H), 7.77-7.53 (m, 3H), 7.39 (dt, J=5.7, 1.8 Hz, 2H), 7.32-7.09 (m, 2H), 7.01-6.82 (m, 1H), 2.67 (q, J=7.2 Hz, 1H), 2.56-2.51 (m, 2H), 1.75-1.44 (m, 2H), 1.22-1.05 (m, 1H), 0.68 (t, J=7.3 Hz, 2H).

### Synthesis of N-[(5-bromothiophen-2-yl)sulfonyl]acetamide

To a flame dried 100 mL round bottom flask equipped with a magnetic stir bar was added 5-bromothiophene-2-sulfonamide (1000 mg, 4.13 mmol), DMAP (5.05 mg, 0.0413 mmol), CH₂Cl₂ (35.0 mL), and THF (5.0 mL). Then the reaction was cooled to 0° C. and via syringe was added pyridine (1.0 mL, 12.4 mmol) followed by acetic anhydride (1.56 mL, 16.5 mmol). The whole slowly became more homogeneous over a few minutes. The reaction was slowly allowed to warm to rt while stirring for 12 h. This crude mixture was diluted with CH₂Cl₂ (25 mL) then washed 3 x with 1 N HCl (20 mL). The organic layer was washed with brine, dried over sodium sulfate, and filtered to give yellow solid after drying under reduced pressure and further on the V-10 (1.07 g, 91% yield). LCMS (220 nm, 254 nm): t_{R} 1.852 min, purity ≥95%, m/z (ESI): 285.70 [M+H]⁺.

### Synthesis of N-{[5-(2,6-diethylphenyl)thiophen-2-yl]sulfonyl}acetamide (RU-NT-363)

In a 50 mL RBF, to a 1,4-dioxane (2 mL) solution of N-[(5-bromothiophen-2-yl)sulfonyl]acetamide (63 mg, 0.22 mmol) 5 mol % Pd(PPh₃)₄ was added and the resulting mixture stirred for 30 min at room temperature under a nitrogen atmosphere. Next, (2,6-diethylphenyl)boronic acid (40 mg, 0.22 mmol), and potassium phosphate (61 mg, 0.45 mmol) were added along with water (1 mL) under a nitrogen atmosphere. The solution was stirred at 95° C. overnight and later cooled to 20° C. Later on H₂O was added and the reaction mixture was extracted with ethyl acetate to obtain an organic layer that was filtered and dried by the addition of MgSO₄. Purification was carried out using preparative HPLC Method B. Solvent was concentrated under vacuo and lyophilized O/N to afford the tile compound as a white solid (5 mg, 7% yield). LCMS (220 nm, 254 nm): t_{R} 2.919 min, purity ≥95%, m/z (ESI): 338.35 [M+H]⁺. ¹H NMR (500 MHz, Methanol-d₄) δ 7.83 (d, J=3.8 Hz, 1H), 7.33 (dd, J=8.0, 7.4 Hz, 1H), 7.17 (d, J=7.7 Hz, 2H), 6.94 (d, J=3.8 Hz, 1H), 2.43 (q, J=7.6 Hz, 4H), 1.09 (t, J=7.6 Hz, 6H).

### Synthesis of methyl 2-amino-3-cyclopropylbenzoate

To a mixture of methyl 2-amino-3-bromobenzoate (3.00 g, 13.0 mmol), cyclopropylboronic acid (1.34 g, 15.6 mmol) and K₂CO₃ (3.59 g, 26.0 mmol) in dioxane/H₂O (v/v=4:1, 30 mL) was added Pd(dppf)Cl₂ DCM (1.06 g, 1.30 mmol) under N₂. Then the reaction mixture was heated to 80° C. and stirred at 80° C. for 12 hours. LCMS showed that starting material was consumed, and the desired product was detected as a major peak. The mixture was diluted with saturated aq. NH₄Cl (50 mL) and extracted with ethyl acetate (50 mL×3). The organic phase was concentrated under reduced pressure. The crude product was purified by column chromatography (SiO₂, ethyl acetate: Petroleum ether=0-10%) to give the title compound (1.47 g, 56.1% yield) as a colorless oil. LCMS (220 nm, 254 nm): t_{R} 1.137 min, purity ≥95%, m/z (ESI): 192.1 [M+H]⁺. HPLC: RT=3.614 min, Area=99.73%. ¹H NMR: (400 MHz, DMSO-d₆) δ 7.63 (dd, J=8.0, 1.2 Hz, 1H), 7.11 (d, J=7.2 Hz, 1H), 6.61 (s, 2H), 6.53-6.46 (m, 1H), 3.79 (s, 3H), 1.74-1.59 (m, 1H), 0.95-0.86 (m, 2H), 0.55-0.45 (m, 2H).

### Synthesis of methyl 2-bromo-3-cyclopropylbenzoate

To a solution of methyl 2-amino-3-cyclopropylbenzoate (2.8 g, 10.61 mmol) in MeCN (50 mL) in a 250 mL RBF was added CuBr (4.74 g, 21.23 mmol) and tert-Butyl nitrite (2.19 g, 21.23 mmol) at 0° C. Then the reaction mixture was heated to 40° C. for 12 hours. The mixture was filtered, and the filtrate was concentrated under reduced pressure. The crude product was purified by column chromatography (SiO₂, ethyl acetate: Petroleum ether=0-15%) to give the titled compound as yellow crystals (2.2 g, 81% yield).

### Synthesis of 2-bromo-3-cyclopropyl-N-methoxy-N-methylbenzamide

To a mixture of 2-bromo-3-cyclopropylbenzoic acid (1.02 g, 4.23 mmol) in a 20 mL scintillation, methoxy(methyl)amine (0.5 g, 5.08 mmol) and DIEA (1.09 g, 8.46 mmol) in DMF (7 mL) was added HATU (1.93 g, 5.08 mmol) at 0° C. Then the reaction mixture was stirred at 20° C. for 1 hour. LCMS showed that the starting material was consumed, and the desired product was detected as a major peak. The mixture was diluted with saturated aq. NH₄Cl (100 mL) and extracted with ethyl acetate (100 mL×3). The organic phase was concentrated under reduced pressure. The crude product was taken directly to the next step. LCMS (220 nm, 254 nm): t_{R} 2.417 min, purity ≥95%, m/z (ESI): 286.0 [M+H]⁺.

### Synthesis of (2-bromo-3-cyclopropylphenyl)(thiophen-2-yl)methanone

To a solution of 2-lithothiophene (1 M in THF) (0.11 mL, 1 eq) in 0.5 mL THF under N₂ in a 10 mL sealed MW vial at −78° C. was added a solution of 2-bromo-3-cyclopropyl-N-methoxy-N-methylbenzamide (40 mg, 0.11 mmol) in dry THF (0.5 mL) was added to the reaction mixture at −78° C. and stirred at −78° C. for 1 hr until completion of the reaction by LCMS. Solvent was evaporated in vacuo and the brown crude solid was taken directly to the next step. LCMS (220 nm, 254 nm): t_{R} 3.128 min, purity ≥95%, m/z (EI): 307.15 [M+H]⁺.

### Synthesis of methyl 5-[2-cyclopropyl-6-(thiophene-2-carbonyl)phenyl]thiophene-2-carboxylate

According to the general procedure for Suzuki coupling reactions using Method A, precatalyst CL. (2-Bromo-3-cyclopropylphenyl)(thiophen-2-yl)methanone (42 mg, 0.14 mmol), [5-(methoxycarbonyl)thiophen-2-yl]boronic acid (25 mg, 0.14 mmol), dry dioxane (2 mL). Temperature: 100° C. Purification was carried out using preparative HPLC Method B. Solvent was concentrated under vacuo and lyophilized O/N to afford the tile compound as an ff-white solid (10 mg, 20% yield). LCMS (220 nm, 254 nm): t_{R} 3.235 min, purity ≥95%, m/z (ESI): 369.15 [M+H]⁺. ¹H NMR (500 MHz, Methanol-d₄) δ 7.85 (dd, J=5.0, 1.2 Hz, 1H), 7.62 (d, J=3.8 Hz, 1H), 7.54-7.44 (m, 1H), 7.37 (dd, J=3.8, 1.2 Hz, 1H), 7.33 (dd, J=7.6, 1.2 Hz, 1H), 7.21 (ddd, J=8.0, 1.3, 0.5 Hz, 1H), 7.10 (dd, J=5.0, 3.8 Hz, 1H), 6.93 (d, J=3.8 Hz, 1H), 4.86 (s, 3H), 1.94-1.84 (m, 1H), 1.02-0.85 (m, 2H), 0.86-0.69 (m, 2H).

### Synthesis of 5-[2-cyclopropyl-6-(thiophene-2-carbonyl)phenyl]thiophene-2-carboxylic acid (RU-NT-366)

According to the general procedure for saponification of methyl esters. Methyl 5-[2-cyclopropyl-6-(thiophene-2-carbonyl)phenyl]thiophene-2-carboxylate (10 mg, 0.03 mmol). Purification was carried out using preparative HPLC Method B. Solvent was concentrated under vacuo and lyophilized O/N to afford the tile compound as a white solid (4.1 mg, 43% yield). LCMS (220 nm, 254 nm): t_{R} 2.788 min, purity ≥95%, m/z (ESI): 355.25 [M+H]⁺. ¹H NMR (500 MHz, DMSO-d₆) δ 13.10 (s, 1H), 8.05 (dd, J=4.9, 1.2 Hz, 1H), 7.58 (d, J=3.8 Hz, 1H), 7.49 (d, J=7.8 Hz, 1H), 7.41 (s, 1H), 7.19 (ddd, J=20.1, 6.4, 2.5 Hz, 3H), 6.96 (d, J=3.8 Hz, 1H), 1.81 (ddd, J=8.4, 5.2, 3.1 Hz, 1H), 0.97-0.83 (m, 2H), 0.77 (dd, J=5.3, 2.0 Hz, 2H).

### Synthesis of N-(2-bromo-3-methylphenyl)-2-(thiophen-2-yl)acetamide

According to the general procedure A for amide coupling. 2-(Thiophen-2-yl)acetic acid (0.2 g, 0.15 mmol), thionyl chloride (1.6 mL), DMF (1 mL). The solution was heated at reflux for 3 hours. 2-Bromo-3-methylaniline (0.25 g, 1.37 mmol), DCM (1 mL). The crude was taken directly to the next step as a blackish solid (0.3 g, 78% yield). LCMS (220 nm, 254 nm): t_{R} 3.423 min, purity ≥95%, m/z (ESI): 310.10 [M+H]⁺.

### Synthesis of methyl 5-{2-methyl-6-[2-(thiophen-2-yl)acetamido]phenyl}thiophene-2-carboxylate

According to the general procedure for Suzuki coupling reactions using Method A, precatalyst C1. N-(2-bromo-3-methylphenyl)-2-(thiophen-2-yl)acetamide (0.4 g, 1.29 mmol), [5-(methoxycarbonyl)thiophen-2-yl]boronic acid (0.24 g, 1.29 mmol), dry dioxane (10 mL). Temperature: 100° C., O/N. Purification was carried out using preparative HPLC Method B. Solvent was concentrated under vacuo. Reddish solid with 89% purity which was taken directly to the next step without further purification (0.23 g, 48% yield). LCMS (220 nm, 254 nm): t_{R} 2.947 min, m/z (ESI): 372.20 [M+H]⁺.

### Synthesis of 5-{2-methyl-6-[2-(thiophen-2-yl)acetamido]phenyl}thiophene-2-carboxylic acid (RU-NT-367)

According to the general procedure for saponification of methyl esters. Methyl 5-{2-methyl-6-[2-(thiophen-2-yl)acetamido]phenyl}thiophene-2-carboxylate (60 mg, 0.16 mmol). Purification was carried out using preparative HPLC Method B. Solvent was concentrated under vacuo to give a white solid (4 mg, 7% yield). LCMS (220 nm, 254 nm): t_{R} 2.453 min, purity ≥95%, m/z (ESI): 399.25 [M+CH₃CN]⁺. ¹H NMR (500 MHz, DMSO-d₆) δ 13.10 (s, 1H), 8.96 (s, 1H), 7.69 (d, J=3.8 Hz, 1H), 7.49 (d, J=8.1 Hz, 1H), 7.42-7.25 (m, 2H), 7.18 (d, J=7.6 Hz, 1H), 7.03-6.88 (m, 1H), 6.77 (d, J=3.4 Hz, 1H), 3.72 (s, 2H), 2.13 (s, 3H).

### Synthesis of methyl 5-{2-methyl-6-[N-methyl-2-(thiophen-2-yl)acetamido]phenyl}thiophene-2-carboxylate

According to the general procedure for N-alkylation of amides (Method A). Methyl 5-{2-methyl-6-[2-(thiophen-2-yl)acetamido]phenyl}thiophene-2-carboxylate (0.15 g, 1e q), K₂CO₃ (80 mg, 0.61 mmol), DMF (1 mL) was stirred at room temperature O/N. Purification was carried out using preparative HPLC Method B. Solvent was concentrated under vacuo to give the desired product with 81% purity as a colorless oil which was taken to the next step directly. LCMS (220 nm, 254 nm, Rt=3.263 min), m/z (ESI): 386.20 [M+H, 100%]⁺.

### Synthesis of 5-{2-methyl-6-[N-methyl-2-(thiophen-2-yl)acetamido]phenyl}thiophene-2-carboxylic acid (RU-NT-368)

According to the general procedure for saponification of methyl esters. Methyl 5-{2-methyl-6-[N-methyl-2-(thiophen-2-yl)acetamido]phenyl}thiophene-2-carboxylate (32 mg, 0.08 mmol). Purification was carried out using preparative HPLC Method B. Solvent was concentrated under vacuo to give the desired product as a white solid (1.3 mg, 4% yield). LCMS (220 nm, 254 nm): t_{R} 2.791 min, purity ≥95%, m/z (ESI): 372.25 [M+H]⁺. ¹H NMR (500 MHz, DMSO-d₆) δ 13.21 (s, 1H), 7.74 (d, J=3.8 Hz, 1H), 7.60-7.41 (m, 2H), 7.38-7.24 (m, 2H), 7.15 (d, J=3.8 Hz, 1H), 6.89 (dd, J=5.2, 3.4 Hz, 1H), 6.71 (dt, J=3.3, 1.1 Hz, 1H), 3.62 (s, 2H), 2.90 (s, 3H), 2.22 (s, 3H).

### Synthesis of N-(2-bromo-3-methylphenyl)-3-methoxy-1,2-oxazole-5-carboxamide

According to the general procedure A for amide coupling. 3-Methoxy-1,2-oxazole-5-carboxylic acid (0.2 g, 1.4 mmol), thionyl chloride (1 mL), and DMF (1 mL). The solution was heated at reflux for 3 hours at 80° C. and the solvent was reduced in vacuo to remove excess thionyl chloride before taking the crude directly to the next step. 2-Bromo-3-methylaniline (0.26 g, 1.4 mmol), DCM (1 mL). Purification was carried out using preparative HPLC Method B. Solvent was concentrated under vacuo to give the desired product as yellow crystals (0.18 g, 41% yield). LCMS (220 nm, 254 nm): t_{R} 2.940 min, purity ≥95%, m/z (ESI): 354.20 [M+CH₃CN, 100%]+. ¹H NMR (500 MHz, DMSO-d₆) δ 10.48 (s, 1H), 7.33 (tt, J=6.9, 3.7 Hz, 3H), 7.01 (s, 1H), 3.98 (s, 3H), 2.41 (s, 3H).

### Synthesis of methyl 5-[2-(3-methoxy-1,2-oxazole-5-amido)-6-methylphenyl]thiophene-2-carboxylate

According to the general procedure for Suzuki coupling reactions using Method A, precatalyst C1. N-(2-bromo-3-methylphenyl)-3-methoxy-1,2-oxazole-5-carboxamide (150 mg, 0.48 mmol), [5-(methoxycarbonyl)thiophen-2-yl]boronic acid (89.67 mg, 0.48 mmol), dry dioxane (10 mL). Temperature; 100° C. Reddish solid (0.15 g, 84% yield) (88% purity). LCMS (220 nm, 254 nm): t_{R} 3.007 min, m/z (ESI): 397.25 [M+Na]⁺.

### Synthesis of methyl 5-[2-methyl-6-(N-methyl-3-methoxy-1,2-oxazole-5-amido)phenyl]thiophene-2-carboxylate

According to the general procedure for N-alkylation of amides (Method A). Methyl 5-[2-(3-methoxy-1,2-oxazole-5-amido)-6-methylphenyl]thiophene-2-carboxylate (100 mg, 1eq), K₂CO₃ (74 mg, 0.54 mmol), DMF (1 mL) was stirred at room temperature overnight. Purification was carried out using preparative HPLC Method B. Solvent was concentrated under vacuo to give the desired product with 89% purity. This was taken to the next step directly without further purification. LCMS (220 nm, 254 nm): t_{R} 2.885 min, m/z (ESI): 387.25 [M+H]⁺.

### Synthesis of 5-[2-methyl-6-(N-methyl-3-methoxy-1,2-oxazole-5-amido)phenyl]thiophene-2-carboxylic acid (RU-NT-369)

According to the general procedure for saponification of methyl esters. Methyl 5-[2-methyl-6-(N-methyl-3-methoxy-1,2-oxazole-5-amido)phenyl]thiophene-2-carboxylate (30 mg, 0.08 mmol). Purification was carried out using preparative HPLC Method B. Solvent was concentrated under vacuo to give the desired product as a white solid (14 mg, 51% yield). LCMS (220 nm, 254 nm): t_{R} 2.423 min, purity ≥95%, m/z (ESI): 373.25 [M+H]⁺. ¹H NMR (500 MHz, DMSO-d₆) δ 13.22 (s, 1H), 7.72 (d, J=3.8 Hz, 1H), 7.46 (d, J=4.7 Hz, 2H), 7.35 (t, J=4.6 Hz, 1H), 6.92 (d, J=3.7 Hz, 1H), 5.74 (s, 1H), 3.84 (s, 3H), 3.06 (s, 3H), 2.18 (s, 3H).

### Synthesis of N-(2-bromo-3-methylphenyl)-5-methylfuran-2-carboxamide

According to the general procedure B for amide coupling. 5-Methylfuran-2-carboxylic acid (0.2 g, 1.59 mmol) 2-bromo-3-methylaniline (295.06 mg, 1.59 mmol), DCM (2 mL). Rtp, O/N. Purification was carried out using preparative HPLC Method B. Solvent was concentrated under vacuo to give the desired product as white crystals (65.9 mg, 14% yield). LCMS (220 nm, 254 nm): t_{R} 3.199 min, m/z (ESI): 296.0 [M+H]⁺. ¹H NMR (300 MHz, DMSO-d₆) δ 9.62 (s, 1H), 7.62-7.41 (m, 1H), 7.38-7.08 (m, 3H), 6.41-6.05 (m, 1H), 2.41 (s, 3H), 2.40-2.37 (m, 3H).

### Synthesis of methyl 5-[2-methyl-6-(5-methylfuran-2-amido)phenyl]thiophene-2-carboxylate

According to the general procedure for Suzuki coupling reactions using Method A, precatalyst C1. N-(2-bromo-3-methylphenyl)-5-methylfuran-2-carboxamide (66 mg, 0.22 mmol), [5-(methoxycarbonyl)thiophen-2-yl]boronic acid (42 mg, 0.22 mmol), dry dioxane (10 mL). Temperature: 130° C. Purification was carried out using preparative HPLC Method B. Solvent was concentrated under vacuo to give the desired product as a reddish solid (92% purity) which was taken directly for the saponification without further purification. LCMS (220 nm, 254 nm): t_{R} 3.183 min, m/z (ESI): 356.20 [M+H]⁺.

### Synthesis of 5-[2-methyl-6-(5-methylfuran-2-amido)phenyl]thiophene-2-carboxylic acid (RU-NT-370)

According to the general procedure for saponification of methyl esters. Methyl 5-[2-methyl-6-(5-methylfuran-2-amido)phenyl]thiophene-2-carboxylate (10 mg, 0.03 mmol). Purification was carried out using preparative HPLC Method B. Solvent was concentrated under vacuo to give a white solid (4.0 mg, 42% yield). LCMS (220 nm, 254 nm): t_{R} 2.588 min, purity ≥95%, m/z (ESI): 342.30 [M+H]⁺. ¹H NMR (500 MHz, DMSO-d₆) δ 13.14 (s, 1H), 9.10 (s, 1H), 7.76 (d, J=3.7 Hz, 1H), 7.53 (d, J=7.9 Hz, 1H), 7.38 (t, J=7.8 Hz, 1H), 7.24 (d, J=7.6 Hz, 1H), 7.11 (d, J=3.8 Hz, 1H), 7.00 (d, J=3.5 Hz, 1H), 6.25 (dd, J=3.4, 1.0 Hz, 1H), 2.27 (s, 3H), 2.19 (s, 3H).

### Synthesis of methyl 5-[2-methyl-6-(N-methyl-5-methylfuran-2-amido)phenyl]thiophene-2-carboxylate

According to the general procedure for N-alkylation of amides (Method A). Methyl 5-[2-methyl-6-(5-methylfuran-2-amido)phenyl]thiophene-2-carboxylate (75 mg, 1 eq), K₂CO₃ (58.3 mg, 0.42 mmol), DMF (1 mL). Rtp, O/N. The crude mixture was taken directly to the next step. LCMS (220 nm, 254 nm): t_{R} 3.0 min, m/z (ESI): 370.15 [M+H]⁺.

### Synthesis of 5-[2-methyl-6-(N-methyl-5-methylfuran-2-amido)phenyl]thiophene-2-carboxylic acid (RU-NT-371)

According to the general procedure for saponification of methyl esters. Methyl 5-[2-methyl-6-(N-methyl-5-methylfuran-2-amido)phenyl]thiophene-2-carboxylate (10 mg, 0.03 mmol). Purification was carried out using preparative HPLC Method B. Solvent was concentrated under vacuo to give a white solid (3.3 mg, 34% yield). LCMS (220 nm, 254 nm): t_{R} 2.541 min, purity ≥95%, m/z (ESI): 356.20 [M+H]⁺. ¹H NMR (500 MHz, DMSO-d₆) δ 13.15 (s, 1H), 7.68 (d, J=3.7 Hz, 1H), 7.60-7.38 (m, 2H), 7.28 (dd, J=6.7, 2.5 Hz, 1H), 6.83 (d, J=3.7 Hz, 1H), 6.03 (d, J=3.4 Hz, 1H), 5.61 (d, J=3.5 Hz, 1H), 3.02 (s, 3H), 2.16 (s, 3H), 2.14 (s, 3H).

### Synthesis of methyl 5-[2-methyl-6-(3-methylthiophene-2-amido)phenyl]thiophene-2-carboxylate

According to the general procedure for Suzuki coupling reactions using Method A, precatalyst C1. N-(2-Bromo-3-methylphenyl)-3-methylthiophene-2-carboxamide (230 mg, 0.42 mmol), [5-(methoxycarbonyl)thiophen-2-yl]boronic acid (77.94 mg, 0.42 mmol), dry dioxane (5 mL). Temperature: 130° C. Purification was carried out using preparative HPLC Method B. Solvent was concentrated under vacuo to give a reddish solid (0.15 g, 96% yield). LCMS (220 nm, 254 nm): t_{R} 3.163 min, purity ≥95%, m/z (ESI): 372.25 [M+H]⁺.

### Synthesis of 5-[2-methyl-6-(3-methylthiophene-2-amido)phenyl]thiophene-2-carboxylic acid (RU-NT-372)

According to the general procedure for saponification of methyl esters. Methyl 5-[2-methyl-6-(3-methylthiophene-2-amido)phenyl]thiophene-2-carboxylate (40 mg, 0.11 mmol). Purification was carried out using preparative HPLC Method B. Solvent was concentrated under vacuo to give a white solid was collected (9.9 mg, 26% yield). LCMS (220 nm, 254 nm): t_{R} 2.653 min, purity ≥95%, m/z (ESI): 358.20 [M+H]⁺. ¹H NMR (500 MHz, DMSO-d₆) δ 13.14 (s, 1H), 8.91 (s, 1H), 7.74 (d, J=3.7 Hz, 1H), 7.69-7.51 (m, 2H), 7.39 (t, J=7.8 Hz, 1H), 7.25 (d, J=7.6 Hz, 1H), 7.09 (d, J=3.8 Hz, 1H), 6.93 (d, J=5.0 Hz, 1H), 2.17 (s, 3H), 2.16 (s, 3H).

### Synthesis of methyl 5-[2-methyl-6-(N-methyl-3-methylthiophene-2-amido)phenyl]thiophene-2-carboxylate

According to the general procedure for N-alkylation of amides (Method A). Methyl 5-[2-methyl-6-(3-methylthiophene-2-amido)phenyl]thiophene-2-carboxylate (100 mg, 1 eq), K₂CO₃ (112 mg, 0.81 mmol) and MeI (0.05 mL, 3 eq) in DMF (1 mL) was stirred at room temperature overnight. Purification was carried out using preparative HPLC Method B. Solvent was concentrated under vacuo to give a reddish solid (80.1 mg, 77% yield, 85% purity). LCMS (220 nm, 254 nm): t_{R} 3.040 min, purity ≥95%, m/z (ESI): 386.15 [M+H]⁺.

### Synthesis of 5-[2-methyl-6-(N-methyl-3-methylthiophene-2-amido)phenyl]thiophene-2-carboxylic acid (RU-NT-373)

According to the general procedure for saponification of methyl esters. Methyl 5-[2-methyl-6-(N-methyl-3-methylthiophene-2-amido)phenyl]thiophene-2-carboxylate (80.1 mg, 0.21 mmol). Purification was carried out using preparative HPLC Method B. Solvent was concentrated under vacuo to give a white solid (54.9 mg, 71% yield). LCMS (220 nm, 254 nm): t_{R} 2.569 min, purity ≥95%, m/z (ESI): 372.20 [M+H]⁺. ¹H NMR (500 MHz, DMSO-d₆) δ 13.18 (s, 1H), 7.70 (d, J=3.7 Hz, 1H), 7.45-7.33 (m, 2H), 7.30 (d, J=7.6 Hz, 1H), 6.79 (d, J=17.6 Hz, 2H), 3.07 (s, 3H), 2.17 (s, 3H), 2.13 (s, 3H).

### Synthesis of N-(2-bromo-3-methylphenyl)-5-methyl-1,3-thiazole-2-carboxamide

According to the general procedure B for amide coupling. 5-Methyl-1,3-thiazole-2-carboxylic acid (0.15 g, 2.1 mmol) and then 2-bromo-3-methylaniline (0.39 g), DCM (5 mL). Rtp, O/N. Purification was carried out using preparative HPLC Method B. Solvent was concentrated under vacuo to give white crystals (50 mg, 8% yield). LCMS (220 nm, 254 nm): t_{R} 3.3325 min, purity ≥95%, m/z (EI): 311.15 [M]⁺.

### Synthesis of methyl 5-[2-methyl-6-(5-methyl-1,3-thiazole-2-amido)phenyl]thiophene-2-carboxylate

According to the general procedure for Suzuki coupling reactions using Method A, precatalyst C1. N-(2-bromo-3-methylphenyl)-5-methyl-1,3-thiazole-2-carboxamide (50 mg, 0.16 mmol), [5-(methoxycarbonyl)thiophen-2-yl]boronic acid (42 mg, 0.22 mmol), dry dioxane (2 mL). Temperature: 100° C. Purification was carried out using preparative HPLC Method B. Solvent was concentrated under vacuo to give a reddish solid (49.9 mg, 83% yield) was taken directly to the next step. LCMS (220 nm, 254 nm): t_{R} 3.303 min, purity ≥95%, m/z (ESI): 373.20 [M+H]⁺.

### Synthesis of 5-[2-methyl-6-(5-methyl-1,3-thiazole-2-amido)phenyl]thiophene-2-carboxylic acid (RU-NT-374)

According to the general procedure for saponification of methyl esters. Methyl 5-[2-methyl-6-(5-methyl-1,3-thiazole-2-amido)phenyl]thiophene-2-carboxylate (10 mg, 0.03 mmol). Purification was carried out using preparative HPLC Method B. Solvent was concentrated under vacuo to give a white solid (3 mg, 31% yield). LCMS (220 nm, 254 nm): t_{R} 2.767 min, purity ≥95%, m/z (ESI): 359.25 [M+H]+. ¹H NMR (500 MHz, DMSO-d₆) δ 13.22 (s, 1H), 9.47 (s, 1H), 7.85 (d, J=8.1 Hz, 1H), 7.79 (d, J=3.8 Hz, 1H), 7.67 (d, J=1.3 Hz, 1H), 7.42 (t, J=7.9 Hz, 1H), 7.24 (d, J=7.6 Hz, 1H), 7.17 (d, J=3.8 Hz, 1H), 2.49 (s, 3H), 2.17 (s, 3H).

### Synthesis of methyl 5-[2-methyl-6-(N-methyl-5-methyl-1,3-thiazole-2-amido)phenyl]thiophene-2-carboxylate

According to the general procedure for N-alkylation of amides (Method A). Methyl-5-[2-methyl-6-(5-methyl-1,3-thiazole-2-amido) phenyl]thiophene-2-carboxylate (40 mg, 1 eq), K₂CO₃ (44.5 mg, 0.32 mmol), DMF (1 mL) was stirred at room temperature overnight. The crude mixture was taken directly to the next step. LCMS (220 nm, 254 nm): t_{R} 3.924 min, m/z (ESI): 387.25 [M+H]⁺.

### Synthesis of 5-[2-methyl-6-(N-methyl-5-methyl-1,3-thiazole-2-amido)phenyl]thiophene-2-carboxylic acid (RU-NT-375)

According to the general procedure for saponification of methyl esters. Crude from the previous step. Purification was carried out using preparative HPLC Method B. Solvent was concentrated under vacuo to give a white solid (2 mg, 5% yield). LCMS (220 nm, 254 nm): t_{R} 3.285 min, purity ≥95%, m/z (ESI): 373.30 [M+H]+. ¹H NMR (300 MHz, DMSO-d₆) δ 9.53 (s, 1H), 8.01-7.85 (m, 1H), 7.81 (d, J=8.1 Hz, 1H), 7.66 (d, J=1.2 Hz, 1H), 7.42 (t, J=7.9 Hz, 1H), 7.25 (d, J=7.6 Hz, 1H), 3.83 (s, 3H), 2.49 (s, 3H), 2.16 (s, 3H).

### Synthesis of N-(2-bromo-3-methylphenyl)-4-methylthiophene-2-carboxamide

According to the general procedure B for amide coupling. 4-Methylthiophene-2-carboxylic acid (0.3 g, 1.87 mmol), 2-bromo-3-methylaniline (0.23 mL, 1.87 mmol), DCM (2 mL). Purification was carried out using preparative HPLC Method B. Solvent was concentrated under vacuo to give a white solid (75.1 mg, 13% yield). LCMS (220 nm, 254 nm): t_{R} 3.060 min, purity ≥95%, m/z (ESI): 312.15 [M+H]+. ¹H NMR (500 MHz, DMSO-d₆) δ 9.92 (s, 1H), 7.81 (d, J=1.5 Hz, 1H), 7.45 (p, J=1.1 Hz, 1H), 7.35 (dd, J=7.7, 2.0 Hz, 1H), 7.31 (d, J=7.5 Hz, 1H), 7.30-7.22 (m, 1H), 2.41 (s, 3H), 2.27 (d, J=1.1 Hz, 3H).

### Synthesis of methyl 5-[2-methyl-6-(4-methylthiophene-2-amido)phenyl]thiophene-2-carboxylate

According to the general procedure for Suzuki coupling reactions using Method A, precatalyst C1. N-(2-N-(2-Bromo-3-methylphenyl)-4-methylthiophene-2-carboxamide (70 mg, 0.23 mmol), [5-(methoxycarbonyl)thiophen-2-yl]boronic acid (42 mg, 0.23 mmol), dry dioxane (2 mL). Purification was carried out using preparative HPLC Method B. Solvent was concentrated under vacuo to give a white solid (86 mg, 96% yield). LCMS (220 nm, 254 nm): t_{R} 3.025 min, purity ≥95%, m/z (ESI): 372.25 [M+H]+.

### Synthesis of 5-[2-methyl-6-(4-methylthiophene-2-amido)phenyl]thiophene-2-carboxylic acid (RU-NT-376)

According to the general procedure for saponification of methyl esters. Methyl 5-[2-methyl-6-(4-methylthiophene-2-amido)phenyl]thiophene-2-carboxylate (10 mg, 0.03 mmol). Purification was carried out using preparative HPLC Method B. Solvent was concentrated under vacuo to give a white solid (4.7 mg, 49% yield). LCMS (220 nm, 254 nm): t_{R} 2.544 min, purity ≥95%, m/z (ESI): 358.25 [M+H]+. ¹H NMR (500 MHz, DMSO-d₆) δ 13.08 (s, 1H), 9.56 (s, 1H), 7.69 (d, J=3.8 Hz, 1H), 7.45 (d, J=1.4 Hz, 1H), 7.38 (dd, J=14.8, 7.1 Hz, 2H), 7.30 (t, J=8.3 Hz, 2H), 7.06 (d, J=3.8 Hz, 1H), 2.20 (s, 3H), 2.19 (s, 3H).

### Synthesis of N-(2-bromo-3-methylphenyl)-N-butyl-5-methylthiophene-2-carboxamide

According to the general procedure for N-alkylation of amides (Method A). N-(2-Bromo-3-methylphenyl)-5-methylthiophene-2-carboxamide (150 mg, 1 eq), K₂CO₃ (200 mg, 1.45 mmol) and excess MeI in DMF (1 mL) was stirred at 80° C. for 2 days. Purification was carried out using preparative HPLC Method B. Solvent was concentrated under vacuo to give a clear oil (27.5 mg, 16% yield). LCMS (220 nm, 254 nm): t_{R} 3.497 min, purity ≥95%, m/z (ESI): 366.20 [M+H]+.

### Synthesis of methyl 5-[2-(N-butyl-5-methylthiophene-2-amido)-6-methylphenyl]thiophene-2-carboxylate

According to the general procedure for Suzuki coupling reactions using Method A, precatalyst C1. N-(2-bromo-3-methylphenyl)-N-butyl-5-methylthiophene-2-carboxamide (27.50 mg, 0.08 mmol), [5-(methoxycarbonyl)thiophen-2-yl]boronic acid (16.75 mg, 0.09 mmol), dry dioxane (2 mL). Temperature: 100° C. The crude material was taken directly to the next step without purification. LCMS (220 nm, 254 nm): t_{R} 3.589 min, purity ≥95%, m/z (ESI): 428.30 [M+H]+.

### Synthesis of 5-[2-(N-butyl-5-methylthiophene-2-amido)-6-methylphenyl]thiophene-2-carboxylic acid (RU-NT-377)

According to the general procedure for saponification of methyl esters. Methyl 5-[2-(N-butyl-5-methylthiophene-2-amido)-6-methylphenyl]thiophene-2-carboxylate crude material from previous step. Purification was carried out using preparative HPLC Method B. Solvent was concentrated under vacuo to give a white solid (5 mg, 12% yield). LCMS (220 nm, 254 nm): t_{R} 3.139 min, purity ≥95%, m/z (ESI): 414.30 [M+H]+. ¹H NMR (500 MHz, DMSO-d₆) δ 13.17 (s, 1H), 7.70 (d, J=3.8 Hz, 1H), 7.56-7.35 (m, 2H), 7.35-7.13 (m, 1H), 6.96 (d, J=3.8 Hz, 1H), 6.63 (d, J=3.8 Hz, 1H), 6.36 (d, J=3.8 Hz, 1H), 3.81 (dt, J=13.0, 7.2 Hz, 2H), 2.34 (s, 3H), 2.20 (s, 3H), 1.56-1.31 (m, 2H), 1.36-1.07 (m, 3H), 0.82 (t, J=7.3 Hz, 2H).

### Synthesis of N-(2-bromo-3-methylphenyl)-2-methyl-1,3-thiazole-5-carboxamide

According to the general procedure B for amide coupling. 2-Methyl-1,3-thiazole-5-carboxylic acid (0.4 g, 2.79 mmol), 2-bromo-3-methylaniline (0.52 g, 2.79 mmol), DCM (3 mL). Rtp, O/N. Purification was carried out using preparative HPLC Method B. Solvent was concentrated under vacuo to give a white solid (0.140 g, 16% yield). LCMS (220 nm, 254 nm): t_{R} 2.880 min, purity ≥95%, m/z (ESI): 351.30 [M+H]⁺.

### Synthesis of methyl 5-[2-methyl-6-(2-methyl-1,3-thiazole-5-amido)phenyl]thiophene-2-carboxylate

According to the general procedure for Suzuki coupling reactions using Method A, precatalyst C1. N-(2-bromo-3-methylphenyl)-2-methyl-1,3-thiazole-5-carboxamide (140 mg, 0.45 mmol), [5-(methoxycarbonyl)thiophen-2-yl]boronic acid (186 mg, 0.45 mmol), dry dioxane (5 mL). Temperature: 100° C. Purification was carried out using preparative HPLC Method B. Solvent was concentrated under vacuo to give a beige solid (60.7 mg, 36% yield). LCMS (220 nm, 254 nm): t_{R} 2.520 min, purity ≥95%, m/z (ESI): 373.25 [M+H]⁺.

### Synthesis of 5-[2-methyl-6-(2-methyl-1,3-thiazole-5-amido)phenyl]thiophene-2-carboxylic acid (RU-NT-380)

According to the general procedure for saponification of methyl esters. Methyl 5-[2-methyl-6-(2-methyl-1,3-thiazole-5-amido)phenyl]thiophene-2-carboxylate (10 mg, 0.03 mmol). Purification was carried out using preparative HPLC Method B. Solvent was concentrated under vacuo to give a white solid (4.3 mg, 45% yield). LCMS (220 nm, 254 nm): t_{R} 2.117 min, purity ≥95%, m/z (ESI): 400.25 [M+CH₃CN]+. ¹H NMR (500 MHz, DMSO-d₆) δ 13.08 (s, 1H), 9.82 (s, 1H), 8.06 (s, 1H), 7.69 (d, J=3.7 Hz, 1H), 7.40 (t, J=7.7 Hz, 1H), 7.31 (dd, J=7.7, 6.3 Hz, 2H), 7.05 (d, J=3.8 Hz, 1H), 2.65 (s, 3H), 2.21 (s, 3H).

### Synthesis of N-(2-bromo-3-methylphenyl)-2-methylthiophene-3-carboxamide

According to the general procedure B for amide coupling. 2-Methylthiophene-3-carboxylic acid (0.3 g, 2.11 mmol), 2-bromo-3-methylaniline (0.39 g, 1 eq), DCM (1 mL). Rtp, O/N. Purification was carried out using preparative HPLC Method B. Solvent was concentrated under vacuo to give white crystals (47.3 mg, 7% yield). LCMS (220 nm, 254 nm): t_{R} 3.064 min, purity ≥95%, m/z (ESI): 312.15 [M+H, 100%]+. ¹H NMR (500 MHz, DMSO-d₆) δ 9.58 (s, 1H), 7.49 (d, J=5.4 Hz, 1H), 7.43-7.35 (m, 2H), 7.30 (t, J=7.7 Hz, 1H), 7.25 (dd, J=7.7, 1.8 Hz, 1H), 2.68 (s, 3H), 2.41 (s, 3H).

### Synthesis of methyl 5-[2-methyl-6-(2-methylthiophene-3-amido)phenyl]thiophene-2-carboxylate

According to the general procedure for Suzuki coupling reactions using Method A, precatalyst C1. N-(2-bromo-3-methylphenyl)-2-methylthiophene-3-carboxamide (45 mg, 0.15 mmol), [5-(methoxycarbonyl)thiophen-2-yl]boronic acid (32.38 mg, 0.17 mmol), dry dioxane (2 mL). Temperature: 100° C. Purification was carried out using preparative HPLC Method B. Solvent was concentrated under vacuo to give a reddish solid (19.5 mg, 36% yield). LCMS (220 nm, 254 nm): t_{R} 3.043 min, purity ≥95%, m/z (ESI): 372.30 [M+H]+.

### Synthesis of 5-[2-methyl-6-(2-methylthiophene-3-amido)phenyl]thiophene-2-carboxylic acid (RU-NT-381)

According to the general procedure for saponification of methyl esters. Methyl 5-[2-methyl-6-(2-methylthiophene-3-amido)phenyl]thiophene-2-carboxylate (5 mg, 0.01 mmol). Purification was carried out using preparative HPLC Method B. Solvent was concentrated under vacuo to give a white solid (1.9 mg, 40% yield). LCMS (220 nm, 254 nm): t_{R} 2.481 min, purity ≥95%, m/z (ESI): 358.25 [M+H]+. ¹H NMR (500 MHz, Methanol-d₄) δ 7.78 (d, J=3.8 Hz, 1H), 7.54 (d, J=8.0 Hz, 1H), 7.40 (t, J=7.8 Hz, 1H), 7.27 (d, J=7.7 Hz, 1H), 7.12 (d, J=5.4 Hz, 1H), 7.04 (d, J=3.8 Hz, 1H), 6.96 (d, J=5.4 Hz, 1H), 2.51 (s, 3H), 2.24 (s, 3H).

### Synthesis of N-(2-bromo-3-methylphenyl)-2-methyl-1,3-thiazole-4-carboxamide

According to the general procedure B for amide coupling. 2-Methyl-1,3-thiazole-4-carboxylic acid (0.3 g, 2.79 mmol), 2-bromo-3-methylaniline (0.52 g, 2.70 mmol), DCM (3 mL). Rtp, O/N. Purification was carried out using preparative HPLC Method B. Solvent was concentrated under vacuo to give a pink solid (180 mg, 21% yield). LCMS (220 nm, 254 nm): t_{R} 3.124 min, purity ≥95%, m/z (ESI): 313.20 [M+H]+. ¹H NMR (500 MHz, DMSO-d₆) δ 9.95 (s, 1H), 8.35 (s, 1H), 8.18-7.98 (m, 1H), 7.32 (t, J=7.8 Hz, 1H), 7.18 (ddd, J=7.5, 1.7, 0.8 Hz, 1H), 2.76 (s, 3H), 2.41 (s, 3H).

### Synthesis of methyl 5-[2-methyl-6-(2-methyl-1,3-thiazole-4-amido)phenyl]thiophene-2-carboxylate

According to the general procedure for Suzuki coupling reactions using Method A, precatalyst C1. N-(2-bromo-3-methylphenyl)-2-methyl-1,3-thiazole-4-carboxamide (150 mg, 0.48 mmol), [5-(methoxycarbonyl)thiophen-2-yl]boronic acid (107.57 mg, 0.58 mmol), dry dioxane (3 mL). Temperature: 110° C. Purification was carried out using preparative HPLC Method B. Solvent was concentrated under vacuo to give a yellowish oil (0.1 g, 56% yield). LCMS (220 nm, 254 nm): t_{R} 3.153 min, purity ≥95%, m/z (ESI): 373.20 [M+H]⁺.

### Synthesis of 5-[2-methyl-6-(2-methyl-1,3-thiazole-4-amido)phenyl]thiophene-2-carboxylic acid (RU-NT-379)

According to the general procedure for Suzuki coupling reactions using Method A, precatalyst CL. Methyl 5-[2-methyl-6-(2-methyl-1,3-thiazole-4-amido)phenyl]thiophene-2-carboxylate (10 mg, 0.03 mmol). Purification was carried out using preparative HPLC Method A. Solvent was concentrated under vacuo prior to lyophilization to give a white solid (7.6 mg, 79% yield). LCMS (220 nm, 254 nm): t_{R} 2.597 min, purity ≥95%, m/z (ESI): 359.25 [M+H]+. ¹H NMR (500 MHz, DMSO-d₆) δ 13.26 (s, 1H), 9.45 (s, 1H), 8.22 (s, 1H), 8.14 (d, J=8.2 Hz, 1H), 7.87 (d, J=3.7 Hz, 1H), 7.41 (t, J=7.9 Hz, 1H), 7.20 (d, J=3.7 Hz, 1H), 7.17 (d, J=7.6 Hz, 2H), 2.55 (s, 3H), 2.18 (s, 3H).

### Synthesis of methyl 5-[2-methyl-6-(N-methyl-2-methyl-1,3-thiazole-4-amido)phenyl]thiophene-2-carboxylate

According to the general procedure for N-alkylation of amides (Method B). Methyl-5-[2-methyl-6-(2-methyl-1,3-thiazole-4-amido)phenyl]thiophene-2-carboxylate (75 mg, 1 eq), DMF (1 mL), room temperature overnight then at 80° C. for 2 hrs. The reaction mixture was quenched with saturated NH₄Cl aqueous solution and extracted with EtOAc and brine 3 times. The organic layers were combined and dried over anhydrous MgSO₄ and concentrate under reduced pressure. The crude mixture was taken directly to the next step without purification. LCMS (220 nm, 254 nm): t_{R} 2.661 min, purity ≥95%, m/z (ESI): 387.25 [M+H]⁺.

### Synthesis of 5-[2-methyl-6-(N-methyl-2-methyl-1,3-thiazole-4-amido)phenyl]thiophene-2-carboxylic acid (RU-NT-382)

According to the general procedure for saponification of methyl esters. Methyl 5-[2-methyl-6-(N-methyl-2-methyl-1,3-thiazole-4-amido)phenyl]thiophene-2-carboxylate (15 mg, 0.04 mmol). Purification was carried out using preparative HPLC Method A. Solvent was concentrated under vacuo prior to lyophilization to give a white solid (10 mg, 69% yield). LCMS (220 nm, 254 nm): t_{R} 2.267 min, purity ≥95%, m/z (ESI): 373.25 [M+H]+. ¹H NMR for the observed rotamers(500 MHz, Methanol-d₄) δ 7.72 (d, J=3.7 Hz, 1H), 7.43-7.19 (m, 2H), 7.26 (s, 1H), 7.16 (dd, J=5.5, 3.7 Hz, 1H), 6.81 (d, J=3.8 Hz, 3H), 3.19 (s, 3H), 2.49 (s, 3H), 2.21 (s, 3H).

### Synthesis of 5-[2-methyl-6-(thiophene-2-amido)phenyl]thiophene-2-carboxylic acid (RU-NT-383)

According to the general procedure for Suzuki coupling reactions using Method A, precatalyst C1. N-(2-bromo-3-methylphenyl)thiophene-2-carboxamide (55.10 mg, 0.19 mmol), [5-(methoxycarbonyl)thiophen-2-yl]boronic acid (38.06 mg, 0.2 mmol), dry dioxane (2 mL). Temperature: 100° C. Purification was carried out using preparative HPLC Method B. Solvent was concentrated under vacuo prior to lyophilization to give a white solid as a (11.3 mg, 18% yield). LCMS (220 nm, 254 nm): t_{R} 2.425 min, purity ≥95%, m/z (ESI): 344.20 [M+H]+. ¹H NMR (500 MHz, DMSO-d₆) δ 13.07 (s, 1H), 9.64 (s, 1H), 7.77 (dd, J=5.0, 1.2 Hz, 1H), 7.69 (d, J=3.7 Hz, 1H), 7.62 (dd, J=3.7, 1.2 Hz, 1H), 7.42-7.37 (m, 1H), 7.34 (d, J=7.2 Hz, 1H), 7.30 (d, J=7.2 Hz, 1H), 7.13 (dd, J=5.0, 3.7 Hz, 1H), 7.06 (d, J=3.8 Hz, 1H), (s, 3H).

### Synthesis of N-(2-bromo-3-methylphenyl)thiophene-2-carboxamide

According to the general procedure A for amide coupling. Thiophene-2-carbonyl chloride (0.3 g, 2.05 mmol), DCM (3 mL). Thiophene-2-carbonyl chloride (0.3 g, 2.05 mmol). Purification was carried out using preparative HPLC Method B. Solvent was concentrated under vacuo to give a yellow oil (180 mg, 30% yield). LCMS (220 nm, 254 nm): t_{R} 3.297 min, purity ≥95%, m/z (ESI): 298.15 [M+H, 100%]+. ¹H NMR (500 MHz, DMSO-d₆) δ 10.03 (s, 1H), 8.00 (dd, J=3.7, 1.1 Hz, 1H), 7.86 (dd, J=5.1, 1.2 Hz, 1H), 7.35 (dd, J=7.7, 2.2 Hz, 1H), 7.32 (t, J=7.5 Hz, 1H), 7.28 (dd, J=7.3, 2.1 Hz, 1H), 7.23 (dd, J=5.0, 3.7 Hz, 1H), 2.42 (s, 3H).

### Synthesis of N-(2-bromo-3-methylphenyl)-N-methylthiophene-2-carboxamide

According to the general procedure for N-alkylation of amides (Method B). N-(2-bromo-3-methylphenyl)thiophene-2-carboxamide (100 mg, 0.34 mmol), DMF (1 mL). Purification was carried out using preparative HPLC Method B. Solvent was concentrated under vacuo to give white crystals (63.8 mg, 61% yield). LCMS (220 nm, 254 nm): t_{R} 2.683 min, purity ≥95%, m/z (ESI): 312.20 [M+H]⁺.

### Synthesis of 5-[2-methyl-6-(N-methylthiophene-2-amido)phenyl]thiophene-2-carboxylic acid (RU-NT-384)

According to the general procedure for Suzuki coupling reactions using Method A, precatalyst C1. N-(2-bromo-3-methylphenyl)-N-methylthiophene-2-carboxamide (63.80 mg, 0.21 mmol), [5-(methoxycarbonyl)thiophen-2-yl]boronic acid (45.90 mg, 0.25 mmol), dry dioxane (3 mL). Temperature: 100° C. Purification was carried out using preparative HPLC Method B. Solvent was concentrated under vacuo to give a white solid after lyophilization (4.6 mg, 6.3% yield). LCMS (220 nm, 254 nm): t_{R} 1.245 min, purity ≥95%, m/z (ESI): 358.210 [M+H]+. ¹H NMR (500 MHz, DMSO-d₆) δ 13.16 (s, 1H), 7.67 (t, J=3.8 Hz, 2H), 7.54-7.46 (m, 2H), 7.46-7.28 (m, 1H), 6.92 (t, J=4.4 Hz, 1H), 6.82 (d, J=3.8 Hz, 1H), 6.59 (d, J=3.8 Hz, 1H), 3.06 (s, 3H), 2.17 (s, 3H).

### Synthesis of 1-(2-bromo-3-methylphenyl)-3-(5-methylthiophen-2-yl)urea

In a 20 mL scintillation vial, 2-bromo-3-methylaniline (220.58 mg, 1.19 mmol) was dissolved in THF (3 mL) and 2-isocyanato-5-methylthiophene (0.248 g, 1.98 mmol) was added to the solution. The reaction mixture was stirred at rtp, O/N. Solvent was reduced in vacuo and the desired product was washed with water and filtered off to give a beige solid (350 mg, 100% yield). LCMS (220 nm, 254 nm): t_{R}2.883 min, purity ≥95%, m/z (ESI): 327.15 [M+H]⁺. ¹H NMR (500 MHz, DMSO-d₆) δ 10.18 (s, 1H), 8.11 (s, 1H), 8.02-7.80 (m, 1H), 7.22 (t, J=7.9 Hz, 1H), 7.03 (d, J=7.4 Hz, 1H), 6.49 (dd, J=3.4, 1.5 Hz, 1H), 6.34 (d, J=3.6 Hz, 1H), 2.37 (s, 3H), 2.33 (s, 3H).

### Synthesis of 1-(2-bromo-3-methylphenyl)-1,3-dimethyl-3-(5-methylthiophen-2-yl)urea

According to the general procedure for N-alkylation of amides (Method B). 1-(2-Bromo-3-methylphenyl)-3-(5-methylthiophen-2-yl)urea (150 mg, 0.46 mmol), DMF (1 mL). Purification was carried out using preparative HPLC Method B. Solvent was concentrated under vacuo to give a yellow oil (0.1 g, 61% yield). LCMS (220 nm, 254 nm): t_{R} 3.137 min, purity ≥95%, m/z (EI): 353.05 [M]⁺.

### Synthesis of 5-(2-methyl-6-{methyl[methyl(5-methylthiophen-2-yl)carbamoyl]amino}phenyl)thiophene-2-carboxylic acid (RU-NT-385)

According to the general procedure for Suzuki coupling reactions using Method A, precatalyst C1. 1-(2-Bromo-3-methylphenyl)-1,3-dimethyl-3-(5-methylthiophen-2-yl)urea (76.1 mg, 0.22 mmol), [5-(methoxycarbonyl)thiophen-2-yl]boronic acid (48 mg, 0.26 mmol), dry dioxane (2 mL). Temperature: 100° C. Purification was carried out using preparative HPLC Method B. Solvent was concentrated under vacuo to give a white solid (6.5 mg, 8% yield). LCMS (220 nm, 254 nm): t_{R} 2.768 min, purity ≥95%, m/z (ESI): 401.30 [M+H]+. ¹H NMR (500 MHz, DMSO-d₆) δ 13.10 (s, 1H), 7.67 (d, J=3.8 Hz, 1H), 7.31-7.09 (m, 2H), 6.99 (d, J=3.7 Hz, 1H), 6.95-6.77 (m, 1H), 6.46-6.22 (m, 1H), 6.07 (d, J=3.6 Hz, 1H), 2.99 (s, 3H), 2.75 (s, 3H), 2.25 (s, 3H), 2.10 (s, 3H).

### Synthesis of 5-{2-[(1-benzothiophen-2-yl)(hydroxy)methyl]-6-cyclopropylphenyl}thiophene-2-carboxylic acid (RU-NT-386)

5-[2-(1-benzothiophene-2-carbonyl)-6-cyclopropylphenyl]thiophene-2-carboxylic acid (10 mg, 0.02 mmol) dissolved in THF (1 mL) and a drop of water was added, sodium borohydride (1 eq) was added in portions at 0° C. and the mixture was refluxed for 2-3 hours at 66° C. The solvent was distilled off under reduced pressure and the residue was extracted with ethyl acetate and brine 3x. The organic layers were combined then dried over MgSO₄, and the solvent was reduced under pressure. Purification was carried out using preparative HPLC Method B. Solvent was concentrated under vacuo to give a white solid (1.1 mg, 11% yield). LCMS (220 nm, 254 nm): t_{R} 2.959 min, purity ≥95%, m/z (ESI): 430.30 [M+Na]+. ¹H NMR (500 MHz, DMSO-d₆) δ 13.15 (s, 1H), 7.85 (d, J=7.7 Hz, 1H), 7.76 (d, J=3.7 Hz, 1H), 7.74-7.69 (m, 1H), 7.48 (d, J=7.7 Hz, 1H), 7.41 (t, J=7.8 Hz, 1H), 7.28 (tt, J=8.6, 6.5 Hz, 1H), 7.04 (s, 1H), 6.95 (d, J=7.6 Hz, 1H), 6.88 (s, 1H), 6.53 (s, 1H), 6.31 (d, J=4.6 Hz, 1H), 5.74 (d, J=4.1 Hz, 1H), 1.58 (td, J=8.5, 4.3 Hz, 1H), 1.19 (d, J=45.3 Hz, 2H), 0.91-0.63 (m, 2H).

### Synthesis of N-(2-bromo-3-methylphenyl)-N-ethyl-5-methylthiophene-2-carboxamide

According to the general procedure for N-alkylation of amides (Method B). N-(2-bromo-3-methylphenyl)-5-methylthiophene-2-carboxamide (100 mg, 0.32 mmol), DMF (1 mL). Colorless oil (33 mg, 30% yield). LCMS (220 nm, 254 nm): t_{R} 3.021 min, purity ≥95%, m/z (EI): 338.20 [M]⁺.

### Synthesis of 5-[2-(N-ethyl-5-methylthiophene-2-amido)-6-methylphenyl]thiophene-2-carboxylic acid (RU-NT-387)

According to the general procedure for Suzuki coupling reactions using Method A, precatalyst C1. N-(2-bromo-3-methylphenyl)-N-ethyl-5-methylthiophene-2-carboxamide (36 mg, 0.11 mmol), [5-(methoxycarbonyl)thiophen-2-yl]boronic acid (23.95 mg, 0.13 mmol), dry dioxane (2 mL). Temperature: 100° C. Purification was carried out using preparative HPLC Method B. Solvent was concentrated under vacuo to give a white solid (5.3 mg, 13% yield). LCMS (220 nm, 254 nm): t_{R} 2.730 min, purity ≥95%, m/z (ESI): 386.25 [M+H]⁺. ¹H NMR (500 MHz, DMSO-d₆) δ 13.16 (s, 1H), 7.69 (d, J=3.8 Hz, 1H), 7.60-7.36 (m, 2H), 7.22 (d, J=7.7 Hz, 1H), 6.95 (d, J=3.8 Hz, 1H), 6.63 (d, J=3.7 Hz, 1H), 6.37 (d, J=3.8 Hz, 1H), 3.84 (q, J=7.0 Hz, 1H), 2.34 (s, 3H), 2.20 (s, 3H), 1.00 (t, J=7.1 Hz, 3H).

### Synthesis of 5-(2-methyl-6-{[(5-methylthiophen-2-yl)carbamoyl]amino}phenyl)thiophene-2-carboxylic acid (RU-NT-388)

According to the general procedure for saponification of methyl esters. Methyl 5-(2-methyl-6-{[(5-methylthiophen-2-yl)carbamoyl]amino}phenyl)thiophene-2-carboxylate (15 mg, 0.04 mmol). Purification was carried out using preparative HPLC Method B. Solvent was concentrated under vacuo to give an off-white solid (5.7 mg, 39% yield). LCMS (220 nm, 254 nm): t_{R} 2.347 min, purity ≥95%, m/z (ESI): 372.25 [M+H]⁺. ¹H NMR (500 MHz, DMSO-d₆) δ 13.21 (s, 1H), 9.81 (s, 1H), 7.94 (d, J=8.3 Hz, 1H), 7.84 (d, J=3.7 Hz, 1H), 7.36 (s, 1H), 7.29 (t, J=8.0 Hz, 1H), 7.10 (d, J=3.7 Hz, 1H), 7.01 (d, J=7.5 Hz, 1H), 6.44 (d, J=3.6 Hz, 1H), 6.23 (d, J=3.6 Hz, 1H), 2.31 (s, 3H), 2.07 (s, 3H).

### Synthesis of methyl 2-amino-3-vinylbenzoate

To a mixture of methyl 2-amino-3-bromobenzoate (5.00 g, 21.7 mmol), vinylboronic acid (4.01 g, 26.0 mmol) and K₂CO₃ (5.99 g, 43.4 mmol) was added Pd(PPh₃)₄ (2.51 g, 2.17 mmol) under N₂ and heated to 100° C. Then the reaction mixture was stirred at 100° C. for 12 hours. LCMS showed that starting material was consumed, and the desired product was detected as a major peak. The mixture was diluted with saturated aq. NH₄Cl (100 mL) and extracted with ethyl acetate (100 mL×3). The organic phase was concentrated under reduced pressure. The crude product was purified by column chromatography (SiO₂, ethyl acetate: Petroleum ether=0-10%) to give the title compound as a yellow oil (3.20 g, 79.3% yield). LCMS (220 nm, 254 nm): t_{R} 0.976 min, purity ≥95%, m/z (ESI): 178.1 [M+H]+. ¹H NMR (400 MHz, DMSO-d₆) δ 7.73 (dd, J=8.0, 1.6 Hz, 1H), 7.51 (dd, J=7.6, 1.6 Hz, 1H), 6.95 (dd, J=17.2, 11.2 Hz, 1H), 6.75 (s, 2H), 6.59 (t, J=7.6 Hz, 1H), 5.66 (dd, J=17.2, 1.6 Hz, 1H), 5.28 (dd, J=11.2, 1.2 Hz, 1H), 3.80 (s, 3H).

### Synthesis of methyl 2-amino-3-ethylbenzoate

To a solution of methyl 2-amino-3-vinylbenzoate (3.20 g, 18.1 mmol) in MeOH (50 mL) was added Pd/C (0.39 g, 3.62 mmol) and stirred under H2 (15 psi) at 20° C. for 12 hours. TLC (Petroleum ether:Ethyl acetate=5:1, R_{f}(R1)=0.50, R_{f}(P1)=0.40) showed that the starting material was consumed, and a new spot was detected. The reaction mixture was filtered, and the filtrate was concentrated under reduced pressure to give the titled compound as a yellow oil (2.85 g, 83% yield). ¹H NMR (400 MHz, DMSO-d₆) δ 7.63 (dd, J=8.0, 1.6 Hz, 1H), 7.18 (d, J=7.2 Hz, 1H), 6.64-6.50 (m, 3H), 3.79 (s, 3H), 2.50 (q, J=7.6 Hz, 2H), 1.15 (t, J=7.6 Hz, 3H).

### Synthesis of methyl 2-bromo-3-ethylbenzoate

To a solution of methyl 2-amino-3-ethylbenzoate (3.35 g, 18.7 mmol) in MeCN (100 mL) was added CuBr (5.37 g, 37.4 mmol) and tert-Butyl nitrite (3.86 g, 37.4 mmol) at 0° C. Then the reaction mixture was heated to 40° C. for 12 hours. TLC (Petroleum ether: ethyl acetate=3:1, R_{f}(R1)=0.20, R_{f}(P1)=0.40) showed that the starting material was consumed, and a new spot was detected. The mixture was filtered, and the filtrate was concentrated under reduced pressure. The crude product was purified by column chromatography (SiO₂, ethyl acetate: Petroleum ether=0-15%) to give the titled compound as a yellow oil. (2.50 g, 52.4% yield). ¹H NMR (400 MHz, DMSO-d₆) δ 7.53-7.38 (m, 3H), 3.86 (s, 3H), 2.78 (q, J=7.6 Hz, 2H), 1.18 (t, J=7.6 Hz, 3H).

### Synthesis of 2-bromo-3-ethylbenzoic acid

To a solution of methyl 2-bromo-3-ethylbenzoate (2.00 g, 8.20 mmol) in MeOH (20 mL) was added a solution of LiOH (0.39 g, 16.4 mmol) in H20 (4 mL) at 0° C. Then the reaction mixture was stirred at 20° C. for 12 hours. TLC (Petroleum ether:ethyl acetate=3:1, R_{f}(R1)=0.60, R_{f}(P1)=0.10) showed that starting material was consumed, and a new spot was detected. The mixture was diluted with water (10 mL) and adjusted pH to 3~4 with aq. HCl (3 mL, 1M). Then the mixture was extracted with DCM (20 mL×3) and concentrated under reduced pressure to give the titled compound as a white solid (1.84 g, 93% yield). ¹H NMR (400 MHz, DMSO-d₆) δ 13.34 (s, 1H), 7.51-7.30 (m, 3H), 2.77 (q, J=7.6 Hz, 2H), 1.17 (t, J=7.6 Hz, 3H).

### Synthesis of 2-bromo-3-ethyl-N-methoxy-N-methylbenzamide

To a mixture of 2-bromo-3-ethylbenzoic acid (1.84 g, 80.0 mmol), N,O-dimethylhydroxylamine (0.94 g, 9.60 mmol) and DIEA (2.07 g, 16.0 mmol) in DMF (20 mL) was added HATU (3.65 g, 9.620 mmol) at 0° C. Then the reaction mixture was stirred at 20° C. for 1 hour. LCMS showed that the starting material was consumed, and the desired product was detected as a major peak. The mixture was diluted with saturated aq. NH₄Cl (100 mL) and extracted with ethyl acetate (100 mL×3). The organic phase was concentrated under reduced pressure. The crude product was purified by column chromatography (SiO₂, Ethyl acetate:Petroleum ether=0-10%) to give the titled compound as a yellow oil (1.82 g, 80% yield). LCMS (220 nm, 254 nm): t_{R} 0.911 min, purity ≥95%, m/z (ESI): 272.0 [M+H]⁺. ¹H NMR (400 MHz, DMSO-d₆) δ 7.46-7.33 (m, 2H), 7.26-7.18 (m, 1H), 3.84-3.38 (m, 3H), 3.31-2.95 (m, 3H), 2.74 (q, J=7.6 Hz, 2H), 1.18 (t, J=7.6 Hz, 3H).

### Synthesis of (5-methylthiophen-2-yl)magnesium bromide

To a stirred mixture of Mg (0.99 g, 40.7 mmol) and 12 (0.43 g, 1.69 mmol) in THF (30 mL) was added a solution of 2-bromo-5-methylthiophene (6.00 g, 33.9 mmol) in THF (30 mL) under N₂ dropwise. Then the reaction mixture was stirred at 20° C. for 3 hours. The reaction was cannot to monitored. The crude product (6.00 g, in 60 mL THF) was used directly for the next step.

### Synthesis of (2-bromo-3-ethylphenyl)(5-methylthiophen-2-yl)methanone

To a solution of 2-bromo-3-ethyl-N-methoxy-N-methylbenzamide (2.28 g, 8.40 mmol) in THF (20 mL) was added (5-methylthiophen-2-yl)magnesium bromide (2.03 g, in 20 mL THF) at 0° C. Then the reaction mixture was stirred at 20° C. for 2 hours. LCMS showed that ~53% start material was remained, and ~46% desired product was detected. The mixture was concentrated under reduced pressure. The crude product was purified by column chromatography (SiO₂, ethyl acetate:Petroleum ether=0-10%) to give the titled compound (1.90 g, 69% yield) as a yellow oil. LCMS (220 nm, 254 nm): t_{R} 1.233 min, purity ≥95%, m/z (ESI): 310.9 [M+H]⁺.

### Synthesis of 5-[2-ethyl-6-(5-methylthiophene-2-carbonyl)phenyl]thiophene-2-carbonitrile

In a 20 mL scintillation vial with a septum was charged with (5-cyanothiophen-2-yl)boronic acid (1.0 eq), Pd(dppf)Cl₂.CH₂Cl₂ (0.1 mol %), and (2-bromo-3-ethylphenyl)(5-methylthiophen-2-yl)methanone (10 mg, 1 eq) in 1:1 (v/v) Toluene/MeOH (1 mL) and was purged with N₂ under vigorous stirring, and 2.0 M aqueous K₂CO₃ solution (2.0 equiv) was added slowly. The mixture was heated at 90° C. overnight. After cooling, purification was carried out using preparative HPLC Method B. Solvent was concentrated under vacuo to give the desired product as a brown solid (10 mg, 18% yield), 90% pure taken directly to the next step without further purification. LCMS (220 nm, 254 nm): t_{R} 3.280 min, m/z (ESI): 338.1 [M+H]⁺.

### Synthesis of 5-{5-[2-ethyl-6-(5-methylthiophene-2-carbonyl)phenyl]thiophen-2-yl}-]H-1,2,3,4-tetrazole (RU-NT-389)

According to the general procedure for tetrazole formation. 5-[2-Ethyl-6-(5-methylthiophene-2-carbonyl)phenyl]thiophene-2-carbonitrile (10 mg, 0.03 mmol). Temperature: 120° C. Purification was carried out using preparative HPLC Method B. Solvent was concentrated under vacuo to give the desired product as a white solid (2 mg, 18% yield). LCMS (220 nm, 254 nm): t_{R} 2.800 min, purity ≥95%, m/z (ESI): 381.30 [M+H]⁺. ¹H NMR (500 MHz, DMSO-d₆) δ 16.95 (s, 1H), 7.67 (d, J=3.7 Hz, 1H), 7.65-7.49 (m, 2H), 7.39 (dd, J=7.3, 1.6 Hz, 1H), 7.25 (d, J=3.7 Hz, 1H), 7.04 (d, J=3.7 Hz, 1H), 6.91 (dd, J=3.8, 1.1 Hz, 1H), 2.61 (q, J=7.5 Hz, 2H), 2.48 (s, 3H), 1.12 (t, J=7.5 Hz, 3H).

### Synthesis of 5-{5,5-dimethyl-4H,5H-naphtho[1,2-b]thiophen-2-yl}-2-methyl-2H-1, 2, 3, 4-tetrazole (RU-NT-390)

To a solution of 5-{5,5-dimethyl-4H,5H-naphtho[1,2-b]thiophen-2-yl}-2H-1,2,3,4-tetrazole (15 mg, 0.05 mmol) in acetone (5 mL) in a 20 mL scintillation vial was added K₂CO₃ (2 eq) followed by addition of MeI (15.08 mg, 2 eq) and the mixture was heated at 60° C. overnight. The reaction mixture was concentrated under vacuo and extraction with brine and EtOAc was carried out 3 times. Purification was carried out using preparative HPLC Method B. Solvent was concentrated under vacuo to give the desired product as a white solid (4.3 mg, 27% yield). LCMS (220 nm, 254 nm): t_{R} 3.387 min, purity ≥95%, m/z (ESI): 297.30 [M+H]⁺. ¹H NMR (500 MHz, MeOD-d₄) δ 7.59 (d, J=0.6 Hz, 1H), 7.43 (ddd, J=7.5, 4.4, 1.9 Hz, 3H), 7.26 (ddd, J=11.6, 8.4, 6.7 Hz, 3H), 4.40 (s, 3H), 2.79 (s, 3H), 1.32 (s, 6H).

### Synthesis of 5-[2-(I-benzothiophene-2-carbonyl)-6-cyclopropylphenyl]thiophene-2-carboxamide

In a 20 mL dram vial was added DIPEA (0.08 mL, 0.48 mmol), PyBOP (91.99 mg, 0.18 mmol) and NH₄Cl (9.45 mg, 0.18 mmol) to a solution of 5-[2-(1-benzothiophene-2-carbonyl)-6-cyclopropylphenyl]thiophene-2-carboxylic acid (8, 0.25 g, 0.76 mmol) in DMSO (1.0 mL) and the reaction was stirred at rtp overnight. The reaction mixture was diluted with water, brine and extracted with EtOAc 3 times. The extract was washed with 5% aqueous HCl, water, dried over anhydrous MgSO₄ and concentrated under reduced pressure. Purification was carried out using preparative HPLC Method B. Solvent was concentrated under vacuo to give the desired product as a white solid (15 mg, 23% yield). LCMS (220 nm, 254 nm): t_{R} 2.905 min, purity ≥95%, m/z (ESI): 404.25 [M+H]⁺.

### Synthesis of 5-[2-(I-benzothiophene-2-carbonyl)-6-cyclopropylphenyl]thiophene-2-carbonitrile

To a solution of 5-[2-(1-benzothiophene-2-carbonyl)-6-cyclopropylphenyl]thiophene-2-carboxamide (15 mg, 1.0 mmol) and 3 drops of pyridine in dry dioxane (1 mL) was added trifluoroacetic anhydride (12 eq) and the reaction stirred at rtp for 2 hrs. Solvent was removed in vacuo and the resultant solid suspended in brine and acidified with 2M HCl(aq) until a pH of 3 was obtained. Extraction using brine and EtOAc 3 times and the organic layers were combined and dried over anhydrous MgSO₄ then reduced in vacuo and the yellow oil (14 mg, 98% yield) was taken to the next step without further purification. LCMS (220 nm, 254 nm): t_{R} 2.910 min, m/z (ESI): 426.25 [M+CH₃CN]+.

### Synthesis of 5-{5-[2-(I-benzothiophene-2-carbonyl)-6-cyclopropylphenyl]thiophen-2-yl}-]H-1,2,3,4-tetrazole (RU-NT-391)

According to the general procedure for tetrazole formation. 5-[2-(1-Benzothiophene-2-carbonyl)-6-cyclopropylphenyl]thiophene-2-carbonitrile (14 mg, 0.04 mmol). Temperature: 120° C. Purification was carried out using preparative HPLC Method B. Solvent was concentrated under vacuo to give the desired product as a white solid (5.2 mg, 33% yield). LCMS (220 nm, 254 nm): t_{R} 3.048 min, purity ≥95%, m/z (ESI): 429.25 [M+H]⁺. ¹H NMR (500 MHz, DMSO-d₆) δ 16.89 (s, 1H), 8.02 (d, J=8.2 Hz, 2H), 7.86 (s, 1H), 7.65 (d, J=3.8 Hz, 1H), 7.56 (t, J=7.7 Hz, 1H), 7.53 (t, J=7.7 Hz, 1H), 7.49 (d, J=7.5 Hz, 1H), 7.44 (t, J=7.6 Hz, 1H), 7.27 (d, J=7.9 Hz, 1H), 7.11 (d, J=3.7 Hz, 1H), 1.89 (td, J=8.5, 4.3 Hz, 1H), 0.91 (dt,J=8.5, 3.1 Hz, 2H), 0.81 (dt, J=5.4, 3.0 Hz, 2H).

### Synthesis of methyl 5-[2-methyl-6-(N-propyl-5-methylthiophene-2-amido)phenyl]thiophene-2-carboxylate

According to the general procedure for N-alkylation of amides (Method B). Methyl 5-[2-methyl-6-(5-methylthiophene-2-amido)phenyl]thiophene-2-carboxylate (74 mg, 0.20 mmol), DMF (1 mL). The crude was taken directly to the next step without further purification. LCMS (220 nm, 254 nm): t_{R} 3.324 min, purity ≥95%, m/z (ESI): 414.30 [M+H]⁺.

### Synthesis of 5-[2-methyl-6-(N-propyl-5-methylthiophene-2-amido)phenyl]thiophene-2-carboxylic acid (RU-NT-392)

According to the general procedure for saponification of methyl esters. Crude material from the previous step. Purification was carried out using preparative HPLC Method B. Solvent was concentrated under vacuo to give the desired product as a white solid (10 mg, 12% yield). LCMS (220 nm, 254 nm): t_{R} 2.879 min, purity ≥95%, m/z (ESI): 400.30 [M+H]⁺. ¹H NMR (500 MHz, DMSO-d₆) δ 13.24 (br s, 1H), 7.83-7.61 (m, 1H), 7.61-7.40 (m, 2H), 7.23 (d, J=7.9 Hz, 1H), 6.95 (d, J=3.6 Hz, 1H), 6.63 (s, 1H), 6.36 (s, 1H), 3.79 (s, 2H), 2.34 (s, 3H), 2.20 (s, 3H), 1.47 (d, J=10.0 Hz, 2H), 0.96-0.69 (m, 3H).

### Synthesis of methyl 5-[2-(N-cyclobutyl-5-methylthiophene-2-amido)-6-methylphenyl]thiophene-2-carboxylate

According to the general procedure for N-alkylation of amides (Method B). Methyl 5-[2-methyl-6-(5-methylthiophene-2-amido)phenyl]thiophene-2-carboxylate (74 mg, 0.20 mmol), DMF (1 mL), excess bromocyclobutane. The crude was taken directly to the next step without further purification. LCMS (220 nm, 254 nm): t_{R} 3.977 min, m/z (ESI): 426.30 [M+H]⁺.

### Synthesis of 5-[2-(N-cyclobutyl-5-methylthiophene-2-amido)-6-methylphenyl]thiophene-2-carboxylic acid (RU-NT-393)

According to the general procedure for saponification of methyl esters. Crude material from the previous step. Purification was carried out using preparative HPLC Method B. Solvent was concentrated under vacuo to give the desired product as a white solid (5.2 mg, 6% yield). LCMS (220 nm, 254 nm): t_{R} 2.892 min, purity ≥95%, m/z (ESI): 412.25 [M+H]⁺. ¹H NMR (500 MHz, DMSO-d₆) δ 13.15 (br s, 1H), 7.68 (s, 1H), 7.61-7.38 (m, 2H), 7.31 (d, J=7.3 Hz, 1H), 6.84 (d, J=3.8 Hz, 1H), 6.62 (s, 1H), 6.27 (s, 1H), 4.34 (d, J=8.5 Hz, 1H), 2.34 (s, 3H), 2.20 (s, 3H), 2.14-2.06 (m, 1H), 1.93 (t, J=9.7 Hz, 1H), 1.87-1.69 (m, 1H), 1.51 (d, J=7.6 Hz, 3H).

### Synthesis of N-(2-chloro-3-ethenylphenyl)-5-methylthiophene-2-carboxamide

According to the general procedure A for amide coupling. Commercially available 5-Methylthiophene-2-carbonyl chloride (126 mg, 0.79 mmol), 2-chloro-3-ethenylaniline (110 mg, 0.72 mmol), DCM (4-5 mL). Rtp, O/N. The solution was concentrated under reduced pressure, and the crude material was purified using preparative HPLC Method B. Solvent was concentrated under vacuo to give the desired product as a white solid reddish oil (21.9 mg, 11% yield). LCMS (220 nm, 254 nm): t_{R} 2.996 min, purity ≥95%, m/z (ESI): 278.20 [M+H]⁺.

### Synthesis of methyl 5-[2-ethenyl-6-(5-methylthiophene-2-amido)phenyl]thiophene-2-carboxylate

According to the general procedure for Suzuki coupling reactions using Method A, precatalyst C1. N-(2-chloro-3-ethenylphenyl)-5-methylthiophene-2-carboxamide (22 mg, 0.08 mmol), [5-(methoxycarbonyl)thiophen-2-yl]boronic acid (17.68 mg, 0.1 mmol), dry dioxane (2 mL). Temperature: 100 C. The crude (30 mg) was taken directly to the next step. LCMS (220 nm, 254 nm): t_{R} 3.057 min, purity ≥95%, m/z (ESI): 384.25 [M+H]⁺.

### Synthesis of methyl 5-[2-ethenyl-6-(N-methyl-5-methylthiophene-2-amido)phenyl]thiophene-2-carboxylate

According to the general procedure for N-alkylation of amides (Method A). Methyl 5-[2-ethenyl-6-(5-methylthiophene-2-amido)phenyl]thiophene-2-carboxylate (31 mg, 1 eq), K₂CO₃ (1.5 eq), acetone (2 mL) was stirred at 50° C. overnight. Purification using preparative HPLC Method B. Solvent was concentrated under vacuo to give the desired product as a which was 70% pure, this was taken directly to the next step without any further purification. LCMS (220 nm, 254 nm): t_{R} 3.070 min, purity ≥95%, m/z (ESI): 398.25 [M+H]⁺.

### Synthesis of 5-[2-ethenyl-6-(N-methyl-5-methylthiophene-2-amido)phenyl]thiophene-2-carboxylic acid (RU-NT-394)

According to the general procedure for saponification of methyl esters. The crude material from the previous step. Purification using preparative HPLC Method B. Solvent was concentrated under vacuo to give the desired product as a white solid (2 mg, 7% yield). LCMS (220 nm, 254 nm): t_{R} 3.644 min, purity ≥95%, m/z (ESI): 384.25 [M+H]⁺. ¹H NMR (500 MHz, MeOD-d₄) δ 7.85 (d, J=7.9 Hz, 1H), 7.67 (d, J=3.7 Hz, 1H), 7.58 (t, J=7.9 Hz, 1H), 7.42 (d, J=7.7 Hz, 1H), 6.89-6.21 (m, 3H), 5.81 (d, J=17.7 Hz, 1H), 5.28 (d, J=11.2 Hz, 1H), 3.17 (s, 3H), 2.37 (s, 3H).

### Synthesis of 2-chloro-3-ethenylaniline

To a cooled solution (0° C.) of zinc dust (12.54 g, 192 mmol) in AcOH (33 mL), THF (33 mL) and water (9.5 mL) was added 2-chloro-1-ethenyl-3-nitrobenzene (2.2 g, 11.98 mmol) and the reaction was allowed to warm to room temperature overnight before vacuum filtration was carried out. The filtrate was diluted with water and EtOAc and the organic layer was collected, dried over MgSO₄ and concentrated in vacuo to afford the title compound as a brownish solid (1.2 g, 65% yield), which was used for the next step without further purification. LCMS (220 nm, 254 nm): t_{R} 2.080 min, purity ≥95%, m/z (ESI): 195.25 [M+CH₃CN, 100%]⁺

### Synthesis of 2-chloro-3-ethylaniline

To a mixture of 2-chloro-1-nitro-3-vinylbenzene (4 g) and 10% Pd/C in EtOAc (10 mL) under an atmosphere of hydrogen (50 psi) at room temperature for 3 hours. The reaction mixture was filtered through a Celite pad. Solvent was concentrated under vacuum. Purification using preparative HPLC Method A. Solvent was concentrated under vacuo to give the desired product as a beige solid after lyophilization (1.5 g, 44% yield). LCMS (220 nm, 254 nm): t_{R} 2.068 min, purity ≥95%, m/z (ESI): 197.25 [M+CH₃CN, 100%]⁺. ¹H NMR (500 MHz, DMSO-d₆) δ 6.95 (t, J=7.7 Hz, 1H), 6.67 (dd, J=8.0, 1.6 Hz, 1H), 6.53 (dd, J=7.5, 1.6 Hz, 1H), 2.62 (q, J=7.5 Hz, 2H), 1.14 (t, J=7.5 Hz, 3H).

### Synthesis of N-(2-chloro-3-ethylphenyl)-5-methylthiophene-2-carboxamide

According to the general procedure A for amide coupling. 2-Chloro-3-ethylaniline (90 mg, 0.58 mmol) and commercially available 5-methylthiophene-2-carbonyl chloride (0.10 g, 0.64 mmol), DCM (2 mL). Rtp O/N. Purification using preparative HPLC Method B. Solvent was concentrated under vacuo to give the desired product as a yellowish semisolid (22.6 mg, 14% yield). LCMS (220 nm, 254 nm): t_{R} 3.153 min, purity ≥95%, m/z (ESI): 280.25 [M+H]⁺.

### Synthesis of 5-[2-ethyl-6-(5-methylthiophene-2-amido)phenyl]thiophene-2-carboxylic acid

According to the general procedure for Suzuki coupling reactions using Method A, precatalyst C1. N-(2-chloro-3-ethylphenyl)-5-methylthiophene-2-carboxamide (22.6 mg, 0.08 mmol), [5-(methoxycarbonyl)thiophen-2-yl]boronic acid (18.03 mg, 0.1 mmol), dry dioxane (2 mL). Temperature: 100° C. The crude was then directly taken to the next step (N-alkylation) without further purification. LCMS (220 nm, 254 nm): t_{R} 2.643 min, m/z (ESI): 372.25 [M+H]⁺.

### Synthesis of methyl 5-[2-ethyl-6-(N-methyl-5-methylthiophene-2-amido)phenyl]thiophene-2-carboxylate

According to the general procedure for N-alkylation of amides (Method A). Methyl-5-[2-ethyl-6-(5-methylthiophene-2-amido)phenyl]thiophene-2-carboxylate (31 mg, 1 eq), K₂CO₃ (21.27 mg, 0.12 mmol), acetone (2 mL) was stirred at 50° C. overnight. Purification using preparative HPLC Method B. Solvent was concentrated under vacuo to give the desired product as a colorless oil (78% pure) which was taken for the saponification without further purification. LCMS (220 nm, 254 nm): t_{R} 3.143 min, purity ≥95%, m/z (ESI): 400.25 [M+H]⁺.

### Synthesis of 5-[2-ethyl-6-(N-methyl-5-methylthiophene-2-amido)phenyl]thiophene-2-carboxylic acid (RU-NT-395)

According to the general procedure for saponification of methyl esters. Methyl 5-[2-ethyl-6-(N-methyl-5-methylthiophene-2-amido)phenyl]thiophene-2-carboxylate crude from previous step. Purification using preparative HPLC Method B. Solvent was concentrated under vacuo to give the desired product as a white solid (5.2 mg, 54% yield). LCMS (220 nm, 254 nm): t_{R} 2.704 min, purity ≥95%, m/z (ESI): 386.20 [M+H]⁺. ¹H NMR (500 MHz, MeOD-d₄) δ 7.63-7.41 (m, 3H), 7.27 (s, 1H), 6.65 (s, 1H), 6.76-6.62 (m, 2H), 3.13 (s, 3H), 2.54 (s, 2H), 2.36 (s, 3H), 1.10 (s, 3H).

### Synthesis of 1-(2-bromo-3-methylphenyl)-3-(5-methylthiophen-2-yl)imidazolidin-2-one

According to the general procedure for N-alkylation of amides (Method B). 1-(2-Bromo-3-methylphenyl)-3-(5-methylthiophen-2-yl)urea (80 mg, 0.25 mmol), THF (2 mL), excess 1,2-dibromoethane. Purification using preparative HPLC Method B. Solvent was concentrated under vacuo to give the desired product as a brownish solid (33 mg, 30% yield). LCMS (220 nm, 254 nm): t_{R} 2.921 min, purity ≥95%, m/z (ESI): 353.15 [M+H]f.

### Synthesis of methyl 5-{2-methyl-6-[3-(5-methylthiophen-2-yl)-2-oxoimidazolidin-]-yl]phenyl}thiophene-2-carboxylate

According to the general procedure for Suzuki coupling reactions using Method A, precatalyst C1. 1-(2-Bromo-3-methylphenyl)-3-(5-methylthiophen-2-yl)imidazolidin-2-one (38 mg, 0.11 mmol), [5-(methoxycarbonyl)thiophen-2-yl]boronic acid (40.24 mg, 0.22 mmol), dry dioxane (1 mL). Purification using preparative HPLC Method B. Solvent was concentrated under vacuo to give the desired product with 90% purity which was taken directly to the next step without further purification. LCMS (220 nm, 254 nm): t_{R} 3.021 min, m/z (ESI): 413.25 [M+H]⁺.

### Synthesis of 5-{2-methyl-6-[3-(5-methylthiophen-2-yl)-2-oxoimidazolidin-1-yl]phenyl}thiophene-2-carboxylic acid (RU-NT-396)

According to the general procedure for saponification of methyl esters. Methyl 5-{2-methyl-6-[3-(5-methylthiophen-2-yl)-2-oxoimidazolidin-1-yl]phenyl}thiophene-2-carboxylate. Crude from previous reaction assuming quantitative yields. Purification using preparative HPLC Method B. Solvent was concentrated under vacuo to give the desired product as a beige solid (8 mg, 18% yield). LCMS (220 nm, 254 nm): t_{R} 2.569 min, purity ≥95%, m/z (ESI): 399.25 [M+H]⁺. ¹H NMR (500 MHz, DMSO-d₆) δ 13.18 (s, 1H), 7.72 (s, 1H), 7.37 (s, 3H), 7.11 (s, 1H), 6.49 (s, 1H), 6.13 (s, 1H), 3.78-3.39 (m, 4H), 2.38-2.08 (m, 6H).

### Synthesis of 1-(2-bromo-3-methylphenyl)-3-(5-methylthiophen-2-yl)urea

In a 20 mL scintillation vial, 2-bromo-3-methylaniline (220.58 mg, 1.19 mmol) was dissolved in THF (3 mL) and 2-isocyanato-5-methylthiophene (0.248 g, 1.98 mmol) was added to the solution. The reaction mixture was stirred at room temperature overnight. Solvent was reduced in vacuo and the desired product was washed with water and filtered off to give a beige solid (350 mg, 100% yield). LCMS (220 nm, 254 nm): t_{R} 2.883 min, purity ≥95%, m/z (ESI): 327.15 [M+H]⁺. ¹H NMR (500 MHz, DMSO-d₆) δ 10.18 (s, 1H), 8.11 (s, 1H), 8.02-7.80 (m, 1H), 7.22 (t, J=7.9 Hz, 1H), 7.03 (d, J=7.4 Hz, 1H), 6.49 (dd, J=3.4, 1.5 Hz, 1H), 6.34 (d, J=3.6 Hz, 1H), 2.37 (s, 3H), 2.33 (s, 3H).

### Synthesis of 1-(2-bromo-3-methylphenyl)-3-(5-methylthiophen-2-yl)-1,3-diazinan-2-one

According to the general procedure for N-alkylation of amides (Method B). 1-(2-Bromo-3-methylphenyl)-3-(5-methylthiophen-2-yl)urea (80 mg, 0.25 mmol), THF (2 mL), excess 1,3-dibromopropane. Purification using preparative HPLC Method B. Solvent was concentrated under vacuo to give the desired product as a white solid (33 mg, 37% yield). LCMS (220 nm, 254 nm): t_{R} 2.905 min, purity ≥95%, m/z (ESI): 367.20 [M+H]⁺. ¹H NMR (500 MHz, DMSO-d₆) δ 7.32 (s, 2H), 7.29-7.18 (m, 1H), 6.50 (dd, J=3.7, 1.5 Hz, 1H), 6.36 (d, J=3.8 Hz, 1H), 3.83 (t, J=6.1 Hz, 2H), 2.40 (s, 2H), 2.29 (d, J=1.2 Hz, 2H).

### Synthesis of methyl 5-{2-methyl-6-[3-(5-methylthiophen-2-yl)-2-oxo-],3-diazinan-]-yl]phenyl}thiophene-2-carboxylate

According to the general procedure for Suzuki coupling reactions using Method A, precatalyst C1. 1-(2-Bromo-3-methylphenyl)-3-(5-methylthiophen-2-yl)-1,3-diazinan-2-one (30 mg, 0.08 mmol), [5-(methoxycarbonyl)thiophen-2-yl]boronic acid (18 mg, 0.1 mmol), dry dioxane (1 mL). Temperature: 100° C. The crude material was taken directly for the saponification. LCMS (220 nm, 254 nm): t_{R} 3.007 min, purity ≥95%, m/z (ESI): 427.30 [M+H]⁺.

### Synthesis of 5-{2-methyl-6-[3-(5-methylthiophen-2-yl)-2-oxo-1,3-diazinan-1-yl]phenyl}thiophene-2-carboxylic acid (RU-NT-397)

According to the general procedure for saponification of methyl esters. Crude from previous reaction assuming quantitative yields. Purification using preparative HPLC Method B. Solvent was concentrated under vacuo to give the desired product as a beige solid (8 mg, 24% yield). LCMS (220 nm, 254 nm): t_{R} 2.519 min, purity ≥95%, m/z (ESI): 413.30 [M+H]⁺. ¹H NMR (500 MHz, DMSO-d₆) δ 13.10 (s, 1H), 7.72 (d, J=3.7 Hz, 1H), 7.41 (s, 1H), 7.37-7.29 (m, 1H), 7.24 (d, J=1.4 Hz, 1H), 7.05 (d, J=3.8 Hz, 1H), 6.64-6.40 (m, 1H), 6.25 (d, J=3.8 Hz, 1H), 3.77-3.46 (m, 3H), 3.14 (dt, J=10.9, 4.9 Hz, 1H), 2.29 (d, J=1.2 Hz, 3H), 2.18 (s, 3H), 2.00 (dt, J=14.2, 5.0 Hz, 1H), 1.53 (dp, J=13.5, 4.5 Hz, 1H).

### Synthesis of N-(2-bromo-3-methylphenyl)-5-ethylthiophene-2-carboxamide

According to the general procedure B for amide coupling. 5-Ethylthiophene-2-carboxylic acid (0.3 g, 1.92 mmol), 2-bromo-3-methylaniline (0.36 g, 1.92 mmol), DCM (3 mL). Temperature: 60° C. Purification using preparative HPLC Method B. Solvent was concentrated under vacuo to give the desired product as white crystals (0.11 g, 18% yield). LCMS (220 nm, 254 nm): t_{R} 3.187 min, purity ≥95%, m/z (EI): 324.15 [M]⁺.

### Synthesis of methyl 5-[2-(5-ethylthiophene-2-amido)-6-methylphenyl]thiophene-2-carboxylate

According to the general procedure for Suzuki coupling reactions using Method A, precatalyst C1. N-(2-bromo-3-methylphenyl)-5-ethylthiophene-2-carboxamide (110 mg, 0.34 mmol), [5-(methoxycarbonyl)thiophen-2-yl]boronic acid (76 mg, 0.41 mmol), dry dioxane (2.26 mL). Purification using preparative HPLC Method B. Solvent was concentrated under vacuo to give the desired product as white crystals (93.7 mg, 72% yield). LCMS (220 nm, 254 nm): t_{R} 3.123 min, purity ≥95%, m/z (ESI): 386.25 [M+H]⁺. ¹H NMR (500 MHz, DMSO-d₆) δ 9.54 (s, 1H), 7.80-7.76 (m, 1H), 7.45 (d, J=3.6 Hz, 1H), 7.39 (dd, J=8.9, 6.7 Hz, 1H), 7.30 (dd, J=15.5, 7.7 Hz, 2H), 7.09 (d, J=3.6 Hz, 1H), 6.85 (d, J=3.6 Hz, 1H), 3.80 (d, J=2.4 Hz, 3H), 2.79 (q, J=7.5 Hz, 2H), 2.20 (d, J=2.1 Hz, 3H), 1.22 (td, J=7.5, 1.4 Hz, 3H).

### Synthesis of 5-[2-(5-ethylthiophene-2-amido)-6-methylphenyl]thiophene-2-carboxylic acid (RU-NT-398)

According to the general procedure for saponification of methyl esters. Methyl 5-[2-(5-ethylthiophene-2-amido)-6-methylphenyl]thiophene-2-carboxylate (15 mg, 0.04 mmol). Purification using preparative HPLC Method B. Solvent was concentrated under vacuo to give the desired product as a white solid (14 mg, 97% yield). LCMS (220 nm, 254 nm): t_{R} 2.637 min, purity ≥95%, m/z (ESI): 372.30 [M+H]⁺. ¹H NMR (500 MHz, DMSO-d₆) δ 13.05 (s, 1H), 9.51 (s, 1H), 7.69 (d, J=3.8 Hz, 1H), 7.51-7.16 (m, 3H), 7.06 (d, J=3.9 Hz, 1H), 6.86 (d, J=3.9 Hz, 1H), 6.53 (s, 1H), 2.80 (q, J=7.9 Hz, 2H), 2.20 (s, 3H), 1.22 (t, J=7.5 Hz, 3H).

### Synthesis of methyl 5-[2-methyl-6-(N-methyl-5-ethylthiophene-2-amido)phenyl]thiophene-2-carboxylate

According to the general procedure for N-alkylation of amides (Method B). Methyl 5-[2-(5-ethylthiophene-2-amido)-6-methylphenyl]thiophene-2-carboxylate (65 mg, mmol), DMF(1 mL). The crude material was taken directly to the next step. LCMS (220 nm, 254 nm): t_{R} 3.152 min, purity ≥95%, m/z (ESI): 400.30 [M+H]⁺.

### Synthesis of 5-[2-methyl-6-(N-methyl-5-ethylthiophene-2-amido)phenyl]thiophene-2-carboxylic acid (RU-NT-399)

According to the general procedure for saponification of methyl esters. Crude from the previous step. Purification using preparative HPLC Method B. Solvent was concentrated under vacuo to give the desired product as a white solid (20 mg, 34% yield). LCMS (220 nm, 254 nm): t_{R} 2.721 min, purity ≥95%, m/z (ESI): 386.25 [M+H]⁺. ¹H NMR (500 MHz, DMSO-d₆) δ 13.15 (s, 1H), 7.67 (d, J=3.7 Hz, 1H), 7.48 (d, J=4.9 Hz, 2H), 7.34 (t, J=4.6 Hz, 1H), 6.85 (d, J=3.8 Hz, 1H), 6.65 (d, J=3.8 Hz, 1H), 6.30 (d, J=3.9 Hz, 1H), 3.03 (s, 3H), 2.70 (q, J=7.5 Hz, 2H), 2.18 (s, 3H), 1.15 (t, J=7.5 Hz, 3H).

### Synthesis of N-(2-bromo-3-methylphenyl)-5-methoxythiophene-2-carboxamide

According to the general procedure B for amide coupling. 5-Methoxythiophene-2-carboxylic acid (0.3 g, 1.9 mmol), 2-bromo-3-methylaniline (0.35 g, 1.9 mmol), DCM (3 mL). Purification using preparative HPLC Method B. Solvent was concentrated under vacuo to give the desired product as off-white crystals (0.14 g, 23% yield). LCMS (220 nm, 254 nm): t_{R} 2.841 min, purity ≥95%, m/z (ESI): 328.15 [M+H]⁺.

### Synthesis of methyl 5-[2-(5-methoxythiophene-2-amido)-6-methylphenyl]thiophene-2-carboxylate & 5-[2-(5-methoxythiophene-2-amido)-6-methylphenyl]thiophene-2-carboxylic acid (RU-NT-400)

According to the general procedure for Suzuki coupling reactions using Method A, precatalyst C1. N-(2-bromo-3-methylphenyl)-5-methoxythiophene-2-carboxamide (100 mg, 0.31 mmol), [5-(methoxycarbonyl)thiophen-2-yl]boronic acid (68 mg, 0.37 mmol), dry dioxane (2 mL). Temperature: 100° C. Purification using preparative HPLC Method B. Solvent was concentrated under vacuo to give the desired ester product as an off-white solid (16.9 mg, 14% yield). LCMS (220 nm, 254 nm): t_{R}2.875 min, purity ≥95%, m/z (ESI): 388.25 [M+H]⁺. Acid side product was afforded as a white solid (60 mg, 52% yield). LCMS (220 nm, 254 nm): t_{R} 2.440 min, purity ≥95%, m/z (ESI): 374.25 [M+H]⁺. ¹H NMR (500 MHz, DMSO-d₆) δ 12.28 (br s, 1H), 8.57 (s, 1H), 6.86 (d, J=3.7 Hz, 1H), 6.62-6.35 (m, 3H), 6.22 (d, J=3.7 Hz, 1H), 5.50 (d, J=4.2 Hz, 1H), 3.05 (s, 3H), 1.36 (s, 3H).

### Synthesis of methyl 5-[2-methyl-6-(N-methyl-5-methoxythiophene-2-amido)phenyl]thiophene-2-carboxylate

According to the general procedure for N-alkylation of amides (Method B). Methyl 5-[2-(5-methoxythiophene-2-amido)-6-methylphenyl]thiophene-2-carboxylate (50 mg, 0.13 mmol), DMF (1 mL). The crude was taken directly to the next step without further purification. LCMS (220 nm, 254 nm): t_{R} 2.891 min, purity ≥95%, m/z (ESI): 402.30 [M+H]⁺.

### Synthesis of 5-[2-methyl-6-(N-methyl-5-methoxythiophene-2-amido)phenyl]thiophene-2-carboxylic acid (RU-NT-401)

According to the general procedure for saponification of methyl esters. Methyl 5-[2-methyl-6-(N-methyl-5-methoxythiophene-2-amido)phenyl]thiophene-2-carboxylate, crude from previous step assuming quantitative yield for the reaction. Purification using preparative HPLC Method B. Solvent was concentrated under vacuo to give the desired product as a white solid (15.2 mg, 31% yield). LCMS (220 nm, 254 nm): t_{R} 2.501 min, purity ≥95%, m/z (ESI): 388.25 [M+H]⁺. ¹H NMR (500 MHz, DMSO-d₆) δ 13.16 (s, 1H), 7.67 (d, J=18.3 Hz, 1H), 7.45-7.23 (m, 3H) 6.87 (s, 1H), 6.10 (s, 2H), 3.79 (d, J=16.9 Hz, 3H), 3.00 (d, J=17.7 Hz, 3H), 2.16 (d, J=16.7 Hz, 3H).

### Synthesis of N-(2-bromo-3-methylphenyl)-5-tert-butylthiophene-2-carboxamide

According to the general procedure B for amide coupling. 5-Tert-butylthiophene-2-carboxylic acid (0.2 g, 1.9 mmol), 2-bromo-3-methylaniline (0.35 g, 1.9 mmol), DCM (5 mL). Purification using preparative HPLC Method B. Solvent was concentrated under vacuo to give the desired product as white crystals (62.2 mg, 9% yield). LCMS (220 nm, 254 nm): t_{R} 3.499 min, purity ≥95%, m/z (ESI): 354.20 [M+H]⁺. ¹H NMR (500 MHz, DMSO-d₆) δ 9.89 (s, 1H), 7.81 (d, J=1.1 Hz, 1H), 7.53-7.13 (m, 2H), 7.01 (dd, J=3.9, 1.3 Hz, 1H), 6.52 (s, 1H), 2.41 (s, 3H), 1.37 (s, 9H).

### Synthesis of methyl 5-[2-(5-tert-butylthiophene-2-amido)-6-methylphenyl]thiophene-2-carboxylate

According to the general procedure for Suzuki coupling reactions using Method A, precatalyst C1. N-(2-bromo-3-methylphenyl)-5-tert-butylthiophene-2-carboxamide (60 mg, 0.17 mmol), [5-(methoxycarbonyl)thiophen-2-yl]boronic acid (34.84 mg, 0.19 mmol), dry dioxane (1 mL). Purification using preparative HPLC Method B. Solvent was concentrated under vacuo to give the desired product as a white solid, (35.3 mg, 65% pure) by NMR. LCMS (220 nm, 254 nm): t_{R} 3.396 min, purity ≥95%, m/z (ESI): 414.30 [M+H]⁺.

### Synthesis of 5-[2-(5-tert-butylthiophene-2-amido)-6-methylphenyl]thiophene-2-carboxylic acid (RU-NT-404)

According to the general procedure for saponification of methyl esters. Methyl 5-[2-(5-tert-butylthiophene-2-amido)-6-methylphenyl]thiophene-2-carboxylate (9 mg, 0.02 mmol). Purification using preparative HPLC Method B. Solvent was concentrated under vacuo to give the desired product as a white solid (8.69 mg, 100% yield). LCMS (220 nm, 254 nm): t_{R} 2.891 min, purity ≥95%, m/z (ESI): 400.30 [M+H]⁺. ¹H NMR (500 MHz, DMSO-d₆) δ 13.09 (s, 1H), 9.49 (s, 1H), 7.70 (d, J=3.7 Hz, 1H), 7.43 (d, J=3.8 Hz, 1H), 7.38 (t, J=7.7 Hz, 1H), 7.36-7.30 (m, 1H), 7.30-7.26 (m, 1H), 7.06 (dd, J=3.8, 2.0 Hz, 1H), 6.91 (d, J=3.9 Hz, 1H), 2.20 (s, 3H), 1.33 (s, 9H).

### Synthesis of methyl 5-[2-methyl-6-(N-methyl-5-tert-butylthiophene-2-amido)phenyl]thiophene-2-carboxylate

According to the general procedure for N-alkylation of amides (Method B). Methyl 5-[2-(5-tert-butylthiophene-2-amido)-6-methylphenyl]thiophene-2-carboxylate (15 mg, 0.06 mmol), DMF (1 mL). The crude material was taken directly for the saponification). LCMS (220 nm, 254 nm): t_{R} 3.419 min, purity ≥95%, m/z (ESI): 428.30 [M+H]⁺.

### Synthesis of 5-[2-methyl-6-(N-methyl-5-tert-butylthiophene-2-amido)phenyl]thiophene-2-carboxylic acid (RU-NT-405)

According to the general procedure for saponification of methyl esters. Crude from the previous step. Purification using preparative HPLC Method B. Solvent was concentrated under vacuo to give the desired product as a white solid (5 mg, 20% yield). LCMS (220 nm, 254 nm): t_{R} 2.985 min, purity ≥95%, m/z (ESI): 414.25 [M+H]⁺. ¹H NMR (500 MHz, Methanol-d₄) δ 7.67 (d, J=3.8 Hz, 1H), 7.62-7.40 (m, 2H), 7.35 (d, J=7.3 Hz, 1H), 6.66 (d, J=3.9 Hz, 2H), 6.49 (d, J=4.0 Hz, 1H), 3.21 (s, 3H, hidden beneath the water signal), 2.22 (s, 3H), 1.31 (s, 9H).

### Synthesis of N-(2-bromo-3-methylphenyl)-4,5-dimethylthiophene-2-carboxamide

According to the general procedure E for amide coupling. 4,5-Dimethylthiophene-2-carboxylic acid (0.3 g, 1.92 mmol), 2-bromo-3-methylaniline (0.43 g, 2.3 mmol), DCM (5 mL). Temperature: 60° C. Purification using preparative HPLC Method B. Solvent was concentrated under vacuo to give the desired product as a white solid (0.129 g, 21% yield). LCMS (220 nm, 254 nm): t_{R} 3.167 min, purity ≥95%, m/z (ESI): 326.10 [M+H]⁺. ¹H NMR (500 MHz, DMSO-d₆) δ 9.76 (s, 1H), 7.70 (s, 1H), 7.35 (dd, J=7.7, 1.9 Hz, 1H), 7.29 (t, J=7.6 Hz, 1H), 7.25 (dd, J=7.5, 1.9 Hz, 1H), 2.40 (s, 3H), 2.36 (s, 3H), 2.14 (s, 3H).

### Synthesis of methyl 5-[2-(4,5-dimethylthiophene-2-amido)-6-methylphenyl]thiophene-2-carboxylate & 5-[2-(4,5-dimethylthiophene-2-amido)-6-methylphenyl]thiophene-2-carboxylic acid (RU-NT-408)

According to the general procedure for Suzuki coupling reactions using Method A, precatalyst C1. N-(2-bromo-3-methylphenyl)-4,5-dimethylthiophene-2-carboxamide (0.1 g, 0.31 mmol), [5-(methoxycarbonyl)thiophen-2-yl]boronic acid (70 mg, 0.37 mmol), dry dioxane (2 mL). Temperature: 100° C. Purification using preparative HPLC Method B. Solvent was concentrated under vacuo to give the desired ester product ester as a white solid (57.6 mg, 48% yield) and the side product which is the saponified product as a white solid (10.3 mg, 9% yield). LCMS (220 nm, 254 nm): t_{R} 3.080 min, purity ≥95%, m/z (ESI): 386.20 [M+H]⁺. LCMS (220 nm, 254 nm): t_{R} 2.605 min, purity ≥95%, m/z (ESI): 327.25 [M+H]⁺. ¹H-NMR of the acid side product (500 MHz, DMSO-d₆) δ 13.08 (br s, 1H), 9.41 (s, 1H), 7.69 (d, J=3.8 Hz, 1H), 7.37 (t, J=7.7 Hz, 1H), 7.34 (s, 1H), 7.32-7.18 (m, 2H), 7.05 (d, J=3.8 Hz, 1H), 2.30 (s, 3H), 2.20 (s, 3H), 2.06 (s, 3H).

### Synthesis of methyl 5-[2-methyl-6-(N-methyl-4,5-dimethylthiophene-2-amido)phenyl]thiophene-2-carboxylate

According to the general procedure for N-alkylation of amides (Method B). Methyl 5-[2-(4,5-dimethylthiophene-2-amido)-6-methylphenyl]thiophene-2-carboxylate (50 mg, 0.13 mmol), DMF (1 mL). Purification using preparative HPLC Method B. Solvent was concentrated under vacuo to give the desired ester product ester as a clear oil (49.6 mg, 96% yield). LCMS (220 nm, 254 nm): t_{R} 3.104 min, purity ≥95%, m/z (ESI): 400.25 [M+H]⁺. ¹H NMR (500 MHz, DMSO-d₆) δ 7.78 (d, J=3.8 Hz, 1H), 7.61-7.39 (m, 2H), 7.39-7.22 (m, 1H), 6.94 (d, J=3.9 Hz, 1H), 6.47 (s, 1H), 3.80 (s, 3H), 2.99 (s, 3H), 2.19 (s, 6H), 1.92 (s, 3H).

### Synthesis of 5-[2-methyl-6-(N-methyl-4,5-dimethylthiophene-2-amido)phenyl]thiophene-2-carboxylic acid (RU-NT-415)

Methyl 5-[2-methyl-6-(N-methyl-4,5-dimethylthiophene-2-amido)phenyl]thiophene-2-carboxylate (40 mg, 0.1 mmol). Purification using preparative HPLC Method B. Solvent was concentrated under vacuo to give the desired ester product as a white solid (18.3 mg, 47% yield). LCMS (220 nm, 254 nm): t_{R} 2.683 min, purity ≥95%, m/z (ESI): 386.25 [M+H]⁺. ¹H NMR (500 MHz, DMSO-d₆) δ 13.15 (br s, 1H), 7.68 (d, J=3.8 Hz, 1H), 7.57-7.37 (m, 2H), 7.30 (dd, J=7.2, 2.0 Hz, 1H), 6.90 (d, J=3.8 Hz, 1H), 6.47 (s, 1H), 2.99 (s, 3H), 2.19 (s, 6H), 1.92 (s, 3H).

### Synthesis of N-(2-bromo-3-methylphenyl)-5-fluorothiophene-2-carboxamide

According to the general procedure C for amide coupling. 5-Fluorothiophene-2-carboxylic acid (0.17 g, 1.16 mmol), 2-bromo-3-methylaniline (0.24 g, 1.28 mmol), EtOAc (5 mL). Purification using preparative HPLC Method B. Solvent was concentrated under vacuo to give the desired ester product as a brownish solid (0.33 g, 90% yield). LCMS (220 nm, 254 nm): t_{R} 2.852 min, purity ≥95%, m/z (ESI): 315.20 [M+H]⁺.

### Synthesis of methyl 5-[2-(5-fluorothiophene-2-amido)-6-methylphenyl]thiophene-2-carboxylate & 5-[2-(5-fluorothiophene-2-amido)-6-methylphenyl]thiophene-2-carboxylic acid (RU-NT-406)

According to the general procedure for Suzuki coupling reactions using Method A, precatalyst C1. N-(2-bromo-3-methylphenyl)-5-fluorothiophene-2-carboxamide (80 mg, 0.25 mmol), [5-(methoxycarbonyl)thiophen-2-yl]boronic acid (71 mg, 0.38 mmol), dry dioxane (2 mL). Temperature: 100° C. Purification using preparative HPLC Method B. Solvent was concentrated under vacuo to give the desired ester product as white crystals (40.2 mg, 42% yield). LCMS (220 nm, 254 nm): t_{R} 2.909 min, purity ≥95%, m/z (ESI): 376.25 [M+H]⁺ and the acid side product as a beige solid (5 mg, 5% yield). LCMS (220 nm, 254 nm): t_{R} 2.452 min, purity ≥95%, m/z (ESI): 362.20 [M+H]⁺. ¹H NMR of the ester: (500 MHz, DMSO-d₆) δ 9.79 (s, 1H), 7.78 (d, J=3.8 Hz, 1H), 7.42 (t, J=4.1 Hz, 1H), 7.39 (d, J=7.8 Hz, 1H), 7.30 (dd, J=11.4, 7.6 Hz, 2H), 7.08 (d, J=3.7 Hz, 1H), 6.79 (dd, J=4.3, 1.8 Hz, 1H), 3.80 (s, 3H), 2.20 (s, 3H). ¹H-NMR of the acid: (500 MHz, DMSO-d₆) δ 13.08 (br s, 1H), 9.76 (s, 1H), 7.69 (d, J=3.8 Hz, 1H), 7.44-7.37 (m, 2H), 7.30 (dd, J=8.3, 7.0 Hz, 2H), 7.04 (d, J=3.8 Hz, 1H), 6.79 (dd, J=4.3, 1.8 Hz, 1H), 2.21 (s, 3H).

### Synthesis of methyl 5-[2-methyl-6-(N-methyl-5-fluorothiophene-2-amido)phenyl]thiophene-2-carboxylate

According to the general procedure for N-alkylation of amides (Method B). Methyl 5-[2-(5-fluorothiophene-2-amido)-6-methylphenyl]thiophene-2-carboxylate (40 mg, 0.11 mmol), DMF (1 mL). Purification using preparative HPLC Method B. Solvent was concentrated under vacuo to give the desired product as a colorless oil (33.4 mg, 81% yield). LCMS (220 nm, 254 nm): t_{R} 2.972 min, purity ≥95%, m/z (ESI): 390.05 [M+H]⁺. ¹H NMR (500 MHz, DMSO-d₆) δ 7.78 (d, J=3.8 Hz, 1H), 7.53 (d, J=6.4 Hz, 2H), 7.43 (dd, J=6.4, 2.9 Hz, 1H), 6.93 (d, J=3.8 Hz, 1H), 6.81-6.52 (m, 1H), 6.16 (t, J=4.1 Hz, 1H), 3.81 (s, 3H), 3.06 (s, 3H), 2.18 (s, 3H).

### Synthesis of 5-[2-methyl-6-(N-methyl-5-fluorothiophene-2-amido)phenyl]thiophene-2-carboxylic acid (RU-NT-416)

According to the general procedure for saponification of methyl esters. Methyl 5-[2-methyl-6-(N-methyl-5-fluorothiophene-2-amido)phenyl]thiophene-2-carboxylate (33.4 mg, 0.09 mmol). Purification using preparative HPLC Method B. Solvent was concentrated under vacuo to give the desired product as a white solid (10 mg, 31% yield). LCMS (220 nm, 254 nm): t_{R} 2.544 min, purity ≥95%, m/z (ESI): 376.00 [M+H]⁺. ¹H NMR (500 MHz, DMSO-d₆) δ 13.19 (br s, 1H), 7.69 (d, J=3.7 Hz, 1H), 7.53 (s, 1H), 7.53-7.41 (m, 2H), 6.89 (d, J=3.8 Hz, 1H), 6.60 (q, J=1.8 Hz, 1H), 6.16 (t, J=4.0 Hz, 1H), 3.06 (s, 3H), 2.18 (s, 3H). ¹⁹F NMR (471 MHz, MeOD) δ-126.84 Hz.

### Synthesis of N-(2-bromo-3-methylphenyl)-4-methyl-1-benzothiophene-2-carboxamide

According to the general procedure B for amide coupling. 4-Methyl-1-benzothiophene-2-carboxylic acid (0.3 g, 1.56 mmol), 2-bromo-3-methylaniline (0.35 g, 1.87 mmol), DCM (5 mL). Temperature: 60° C. O/N. Purification using preparative HPLC Method B. Solvent was concentrated under vacuo to give the desired product as a white solid (44 mg, 8% yield). LCMS (220 nm, 254 nm): t_{R} 3.385 min, purity ≥95%, m/z (EI): 360.15 [M]⁺. ¹H NMR (500 MHz, DMSO-d₆) δ 10.33 (s, 1H), 8.49 (d, J=0.9 Hz, 1H), 7.87 (d, J=8.1 Hz, 1H), 7.65-6.74 (m, 4H), 2.64 (s, 3H), 2.44 (s, 3H).

### Synthesis of methyl 5-[2-methyl-6-(4-methyl-1-benzothiophene-2-amido)phenyl]thiophene-2-carboxylate & 5-[2-methyl-6-(4-methyl-1-benzothiophene-2-amido)phenyl]thiophene-2-carboxylic acid (RU-NT-407)

According to the general procedure for Suzuki coupling reactions using Method A, precatalyst C1. N-(2-bromo-3-methylphenyl)-4-methyl-1-benzothiophene-2-carboxamide (42 mg, 0.12 mmol), [5-(methoxycarbonyl)thiophen-2-yl]boronic acid (32.52 mg, 0.17 mmol), dry dioxane (1 mL). Temperature: 100° C. Purification using preparative HPLC Method B. Solvent was concentrated under vacuo to give the desired ester product as a white solid (30 mg, 61% yield), and the saponified side product as a white solid (20 mg, 42% yield). LCMS of the ester (220 nm, 254 nm): t_{R} 3.349 min, purity ≥95%, m/z (ESI): 422.30 [M+H]⁺. LCMS of the acid side product (220 nm, 254 nm): t_{R} 2.867 min, purity ≥95%, m/z (ESI): 408.30 [M+H]⁺. ¹H NMR (500 MHz, DMSO-d₆) δ 13.06 (br s, 1H), 9.95 (s, 1H), 8.04 (s, 1H), 7.82 (d, J=8.1 Hz, 1H), 7.70 (d, J=3.7 Hz, 1H), 7.42 (t, J=7.7 Hz, 1H), 7.36 (d, J=7.9 Hz, 1H), 7.35-7.30 (m, 3H), 7.23 (d, J=7.2 Hz, 1H), 7.10 (d, J=3.8 Hz, 1H), 2.55 (s, 3H), 2.24 (s, 3H).

### Synthesis of methyl 5-[2-methyl-6-(N-methyl-4-methyl-1-benzothiophene-2-amido)phenyl]thiophene-2-carboxylate

According to the general procedure for N-alkylation of amides (Method B). Methyl 5-[2-methyl-6-(4-methyl-1-benzothiophene-2-amido)phenyl]thiophene-2-carboxylate (50 mg, 0.12 mmol), DMF (1 mL). Purification using preparative HPLC Method B. Solvent was concentrated under vacuo to give the desired ester product as a clear oil (12 mg, 23% yield). LCMS of the ester (220 nm, 254 nm): t_{R} 3.352 min, purity ≥95%, m/z (ESI): 436.30 [M+H]⁺. ¹H NMR (500 MHz, DMSO-d₆) δ 7.77 (d, J=3.8 Hz, 1H), 7.72 (d, J=8.2 Hz, 1H), 7.55 (d, J=6.1 Hz, 2H), 7.47 (d, J=6.2 Hz, 1H), 7.30 (t, J=7.7 Hz, 1H), 7.15 (d, J=7.2 Hz, 1H), 6.90 (d, J=3.9 Hz, 1H), 6.86 (s, 1H), 6.52 (s, 1H), 3.78 (s, 3H), 3.13 (s, 3H), 2.31 (s, 3H), 2.19 (s, 3H).

### Synthesis of 5-[2-methyl-6-(N-methyl-4-methyl-1-benzothiophene-2-amido)phenyl]thiophene-2-carboxylic acid (RU-NT-417)

According to the general procedure for saponification of methyl esters. Crude material from the previous step. Purification using preparative HPLC Method B. Solvent was concentrated under vacuo to give the desired ester product as a white solid (3.6 mg, 7% yield), assuming quantitative yields from the previous step. LCMS (220 nm, 254 nm): t_{R} 2.924 min, purity ≥95%, m/z (ESI): 422.20 [M+H]⁺. ¹H NMR (500 MHz, DMSO-d₆) δ 13.17 (br s, 1H), 7.72 (d, J=8.2 Hz, 1H), 7.67 (d, J=3.8 Hz, 1H), 7.58-7.49 (m, 2H), 7.47 (dd, J=6.6, 2.6 Hz, 1H), 7.41-7.22 (m, 1H), 7.14 (d, J=7.2 Hz, 1H), 6.85 (t, J=1.9 Hz, 2H), 3.13 (s, 3H), 2.31 (s, 3H), 2.19 (s, 3H).

### Synthesis of N-(2-bromo-3-methylphenyl)-4H,6H, 7H-thieno[3,2-c]pyran-2-carboxamide

According to the general procedure D for amide coupling. 4H,6H,7H-Thieno[3,2-c]pyran-2-carboxylic acid (0.25 g, 1.36 mmol), tetrahydrofuran (THF) (6 mL), 2-bromo-3-methylaniline (0.31 g, 1.63 mmol). Purification using preparative HPLC Method B. Solvent was concentrated under vacuo to give the desired ester product as white crystals (50.3 mg, 10% yield). LCMS (220 nm, 254 nm): t_{R}2.721 min, purity ≥95%, m/z (ESI): 354.15 [M+H]⁺. ¹H NMR (500 MHz, DMSO-d₆) δ 9.89 (s, 1H), 7.68 (s, 1H), 7.35 (dd, J=7.8, 1.9 Hz, 1H), 7.30 (t, J=7.6 Hz, 1H), 7.27 (dd, J=7.6, 1.9 Hz, 1H), 4.65 (d, J=2.1 Hz, 2H), 3.90 (t, J=5.5 Hz, 2H), 2.86 (t, J=5.6 Hz, 2H), 2.41 (s, 3H).

### Synthesis of methyl 5-(2-methyl-6-{4H,6H, 7H-thieno[3,2-c]pyran-2-amido}phenyl)thiophene-2-carboxylate

According to the general procedure for Suzuki coupling reactions using Method A, precatalyst C1. N-(2-bromo-3-methylphenyl)-4H,6H,7H-thieno[3,2-c]pyran-2-carboxamide (48 mg, 0.14 mmol), [5-(methoxycarbonyl)thiophen-2-yl]boronic acid (30.41 mg, 0.16 mmol), dry dioxane (2 mL). Temperature: 100° C. Purification using preparative HPLC Method B. Solvent was concentrated under vacuo to give the desired ester product as a yellow oil (32.2 mg, 57% yield). LCMS (220 nm, 254 nm): t_{R} 2.763 min, purity ≥95%, m/z (ESI): 414.25 [M+H]⁺.

### Synthesis of 5-(2-methyl-6-{4H,6H, 7H-thieno[3,2-c]pyran-2-amido}phenyl)thiophene-2-carboxylic acid (RU-NT-411)

According to the general procedure for saponification of methyl esters. Methyl 5-(2-methyl-6-{4H,6H,7H-thieno[3,2-c]pyran-2-amido}phenyl)thiophene-2-carboxylate (8 mg, 0.02 mmol). Purification using preparative HPLC Method B. Solvent was concentrated under vacuo to give the desired ester product as a white solid. (6.3 mg, 82% yield). LCMS (220 nm, 254 nm): t_{R} 2.336 min, purity ≥95%, m/z (ESI): 400.25 [M+H]⁺. ¹H NMR (500 MHz, DMSO-d₆) δ 9.56 (s, 1H), 7.69 (d, J=3.7 Hz, 1H), 7.38 (dd, J=8.4, 7.1 Hz, 1H), 7.34 (s, 1H), 7.29 (d, J=7.8 Hz, 2H), 7.04 (d, J=3.8 Hz, 1H), 4.57 (s, 2H), 3.86 (t, J=5.5 Hz, 2H), 2.81 (t, J=5.7 Hz, 2H), 2.20 (s, 3H).

### Synthesis of methyl 5-(2-methyl-6-{N-methyl-4H,6H, 7H-thieno[3,2-c]pyran-2-amido}phenyl)thiophene-2-carboxylate

According to the general procedure for N-alkylation of amides (Method B). Methyl 5-(2-methyl-6-{4H,6H,7H-thieno[3,2-c]pyran-2-amido}phenyl)thiophene-2-carboxylate (24 mg, 0.06 mmol), DMF (1 mL). The crude was taken directly to the next step without further purification. LCMS (220 nm, 254 nm): t_{R} 2.769 min, purity ≥95%, m/z (ESI): 428.35 [M+H]⁺.

### Synthesis of 5-(2-methyl-6-{N-methyl-4H,6H, 7H-thieno[3,2-c]pyran-2-amido}phenyl)thiophene-2-carboxylic acid (RU-NT-418)

According to the general procedure for saponification of methyl esters. Crude from the previous step. Purification using preparative HPLC Method B. Solvent was concentrated under vacuo to give the desired ester product as a white solid (6.5 mg, 26% yield), quantitative yield assumed from previous step. LCMS (220 nm, 254 nm): t_{R} 2.377 min, purity ≥95%, m/z (ESI): 414.30 [M+H]⁺. ¹H NMR (500 MHz, MeOD-d₄) δ 7.69 (d, J=3.8 Hz, 1H), 7.48 (d, J=4.8 Hz, 2H), 7.33 (t, J=4.6 Hz, 1H), 6.75 (d, J=3.8 Hz, 1H), 6.60 (s, 1H), 4.50 (s, 2H), 3.88 (d, J=5.5 Hz, 2H), 3.17 (s, 3H), 2.76 (t, J=5.6 Hz, 2H), 2.23 (s, 3H).

### Synthesis of methyl 5-[2-methyl-6-(4,5,6,7-tetrahydro-]-benzothiophene-2-amido)phenyl]thiophene-2-carboxylate

According to the general procedure for Suzuki coupling reactions using Method A, precatalyst C1. N-(2-bromo-3-methylphenyl)-4,5,6,7-tetrahydro-1-benzothiophene-2-carboxamide (60 mg, 0.17 mmol), [5-(methoxycarbonyl)thiophen-2-yl]boronic acid (38.23 mg, 0.21 mmol), dry dioxane (2 mL). Temperature: 100° C. Purification using preparative HPLC Method B. Solvent was concentrated under vacuo to give the desired ester product as a yellow oil (83.2 mg, 90% yield). LCMS (220 nm, 254 nm): t_{R} 3.300 min, purity ≥95%, m/z (ESI): 412.30 [M+H]⁺. ¹H NMR (500 MHz, DMSO-d₆) δ 9.49 (s, 1H), 7.78 (d, J=3.8 Hz, 1H), 7.38 (dd, J=8.3, 7.2 Hz, 1H), 7.33-7.24 (m, 2H), 7.08 (d, J=3.8 Hz, 1H), 3.80 (s, 3H), 2.75 (s, 1H), 2.70 (t, J=6.0 Hz, 2H), 2.59-2.51 (m, 2H), 2.19 (s, 3H), 1.92-1.52 (m, 4H).

### Synthesis of 5-[2-methyl-6-(4,5,6,7-tetrahydro-]-benzothiophene-2-amido)phenyl]thiophene-2-carboxylic acid (RU-NT-414)

Methyl 5-[2-methyl-6-(4,5,6,7-tetrahydro-1-benzothiophene-2-amido)phenyl]thiophene-2-carboxylate (10 mg, 0.02 mmol). Purification using preparative HPLC Method B. Solvent was concentrated under vacuo to give the desired product as a white solid (7 mg, 73% yield). LCMS (220 nm, 254 nm): t_{R} 2.803 min, purity ≥95%, m/z (ESI): 398.30 [M+H]⁺. ¹H NMR (500 MHz, DMSO-d₆) δ 13.08 (br s, 1H), 9.44 (s, 1H), 7.70 (d, J=3.8 Hz, 1H), 7.38 (t, J=7.7 Hz, 1H), 7.34-7.24 (m, 3H), 7.05 (dd, J=3.7, 2.0 Hz, 1H), 2.71 (t, J=6.1 Hz, 2H), 2.53 (t, J=6.3 Hz, 2H), 2.20 (s, 3H), 1.73 (dtt, J=20.6, 5.3, 3.2 Hz, 3H).

### Synthesis of methyl 5-[2-methyl-6-(N-methyl-4,5,6,7-tetrahydro-1-benzothiophene-2-amido)phenyl]thiophene-2-carboxylate

According to the general procedure for N-alkylation of amides (Method A). Methyl 5-[2-methyl-6-(4,5,6,7-tetrahydro-1-benzothiophene-2-amido)phenyl]thiophene-2-carboxylate (50 mg, 1 eq), K₂CO₃ (50.37 mg, 3 eq), anhydrous THF (1 mL). Temperature: 70° C. The crude mixture was taken directly to the next step. LCMS (220 nm, 254 nm): t_{R} 3.335 min, purity ≥95%, m/z (ESI): 426.35 [M+H]⁺.

### Synthesis of 5-[2-methyl-6-(N-methyl-4,5,6,7-tetrahydro-1-benzothiophene-2-amido)phenyl]thiophene-2-carboxylic acid (RU-NT-420)

According to the general procedure for saponification of methyl esters. Crude from the previous step. Purification using preparative HPLC Method B. Solvent was concentrated under vacuo to give the desired product as a white solid (3.4 mg, 7% yield), quantitative yield assumed from previous step. LCMS (220 nm, 254 nm): t_{R} 2.897 min, purity ≥95%, m/z (ESI): 412.35 [M+H]⁺. ¹H NMR (500 MHz, Methanol-d₄) δ 7.69 (d, J=3.7 Hz, 1H), 7.48 (d, J=4.9 Hz, 2H), 7.30 (t, J=4.6 Hz, 1H), 6.74 (d, J=3.7 Hz, 1H), 6.55 (s, 1H), 3.16 (s, 3H), 2.65 (d, J=6.8 Hz, 2H), 2.43 (s, 2H), 2.23 (s, 3H), 1.76 (dd, J=25.6, 6.9 Hz, 2H), 1.31 (d, J=22.6 Hz, 2H).

### Synthesis of N-(2-bromo-3-methylphenyl)-3-methyl-N-propylthiophene-2-carboxamide

According to the general procedure for N-alkylation of amides (Method B). N-(2-bromo-3-methylphenyl)-3-methylthiophene-2-carboxamide (0.25 g, 0.81 mmol), DMF (1 mL) excess 1-iodopropane. Purification using preparative HPLC Method B. Solvent was concentrated under vacuo to give the desired product as a yellow oil (95.7 mg, 34% yield). LCMS (220 nm, 254 nm): t_{R} 3.199 min, purity ≥95%, m/z (ESI): 352.20 [M+H]⁺. ¹H NMR (500 MHz, DMSO-d₆) δ 7.32 (d, J=7.7 Hz, 2H), 7.28 (t, J=7.8 Hz, 1H), 7.19 (d, J=7.7 Hz, 1H), 6.77 (s, 1H), 2.35 (s, 2H), 2.33 (s, 3H), 1.61-1.44 (m, 2H), 0.93-0.79 (m, 3H).

### Synthesis of 5-[2-methyl-6-(N-propyl-3-methylthiophene-2-amido)phenyl]thiophene-2-carboxylic acid (RU-NT-421)

According to the general procedure for Suzuki coupling reactions using Method A, precatalyst C1. N-(2-bromo-3-methylphenyl)-3-methyl-N-propylthiophene-2-carboxamide (93 mg, 0.26 mmol), [5-(methoxycarbonyl)thiophen-2-yl]boronic acid (58.92 mg, 0.32 mmol), dry dioxane (3 mL). Temperature: 120° C. Purification using preparative HPLC Method B. Solvent was concentrated under vacuo to give the desired product as a white solid (7.3 mg, 7% yield). LCMS (220 nm, 254 nm): t_{R} 2.831 min, purity ≥95%, m/z (ESI): 400.30 [M+H]⁺. ¹H NMR (500 MHz, DMSO-d₆) δ 13.17 (br s, 1H), 7.73 (d, J=3.8 Hz, 1H), 7.39 (d, J=23.5 Hz, 3H), 7.13 (s, 1H), 6.92 (s, 1H), 6.82 (d, J=4.7 Hz, 1H), 2.26 (s, 3H), 2.15 (s, 3H), 1.47 (d, J=14.0 Hz, 2H), 0.83 (d, J=27.9 Hz, 3H).

### Synthesis of N-(2-bromo-3-methylphenyl)-3-methyl-1-benzothiophene-2-carboxamide

According to the general procedure B for amide coupling. 3-Methyl-1-benzothiophene-2-carboxylic acid (0.3 g, 1.56 mmol), 2-bromo-3-methylaniline (0.35 g, 1.87 mmol), DCM (3 mL). Purification using preparative HPLC Method B. Solvent was concentrated under vacuo to give the desired product as white crystals (26.1 mg, 5% yield). LCMS (220 nm, 254 nm): t_{R} 3.541 min, purity ≥95%, m/z (ESI): 362.10 [M+H]⁺.

### Synthesis of methyl 5-[2-methyl-6-(3-methyl-1-benzothiophene-2-amido)phenyl]thiophene-2-carboxylate

According to the general procedure for Suzuki coupling reactions using Method A, precatalyst C1. N-(2-bromo-3-methylphenyl)-3-methyl-1-benzothiophene-2-carboxamide (26.10 mg, 0.07 mmol), [5-(methoxycarbonyl)thiophen-2-yl]boronic acid (20.21 mg, 0.11 mmol), dry dioxane (1 mL). Purification using preparative HPLC Method B. Solvent was concentrated under vacuo to give the desired product a yellow oil (14.9 mg, 49% yield). LCMS (220 nm, 254 nm): t_{R} 3.392 min, purity ≥95%, m/z (ESI): 422.25 [M+H]⁺.

### Synthesis of 5-[2-methyl-6-(3-methyl-1-benzothiophene-2-amido)phenyl]thiophene-2-carboxylic acid (RU-NT-412)

Methyl 5-[2-methyl-6-(3-methyl-1-benzothiophene-2-amido)phenyl]thiophene-2-carboxylate (4.9 mg, 0.01 mmol). Purification using preparative HPLC Method B. Solvent was concentrated under vacuo to give the desired product as a white solid. (4 mg, 84% yield). LCMS (220 nm, 254 nm): t_{R} 2.913 min, purity ≥95%, m/z (ESI): 408.30 [M+H]⁺. ¹H NMR (500 MHz, DMSO-d₆) δ 13.11 (s, 1H), 9.44 (br s, 1H), 8.03-7.95 (m, 1H), 7.93-7.81 (m, 1H), 7.75 (d, J=3.8 Hz, 1H), 7.59-7.37 (m, 4H), 7.30 (d, J=7.6 Hz, 1H), 7.10 (d, J=3.8 Hz, 1H), 2.35 (s, 3H), 2.20 (s, 3H).

### Synthesis of methyl 5-[2-methyl-6-(N-methyl-3-methyl-]-benzothiophene-2-amido)phenyl]thiophene-2-carboxylate

According to the general procedure for N-alkylation of amides (Method B). Methyl 5-[2-methyl-6-(3-methyl-1-benzothiophene-2-amido)phenyl]thiophene-2-carboxylate (10 mg, 0.02 mmol), THF (1 mL). The crude material was taken directly to the next step without further purification. LCMS (220 nm, 254 nm): t_{R} 3.753 min, purity ≥95%, m/z (ESI): 436.25 [M+H]⁺.

### Synthesis of 5-[2-methyl-6-(N-methyl-3-methyl-1-benzothiophene-2-amido)phenyl]thiophene-2-carboxylic acid (RU-NT-422)

According to the general procedure for saponification of methyl esters. Crude from the previous step. Purification using preparative HPLC Method B. Solvent was concentrated under vacuo to give the desired product as a white solid (5.6 mg, 58% yield), quantitative yield assumed from previous step. LCMS (220 nm, 254 nm): t_{R} 2.803 min, purity ≥95%, m/z (ESI): 422.30 [M+H]⁺. ¹H NMR for the observed rotamers (500 MHz, DMSO-d₆) δ 13.19 (s, 1H), 7.94-7.70 (m, 3H), 7.56-7.17 (m, 5H), 6.87 (d, J=3.8 Hz, 1H), 3.06 (s, 3H), 2.34 (s, 3H), 2.12 (s, 3H).

### Synthesis of methyl 5-(2-methyl-6-{N-methyl-2H,3H-thieno[2,3-b] [1,4]dioxine-6-amido}phenyl)thiophene-2-carboxylate

According to the general procedure for N-alkylation of amides (Method B). Methyl 5-(2-methyl-6-{2H,3H-thieno[2,3-b][1,4]dioxine-6-amido}phenyl)thiophene-2-carboxylate (6 mg, 0.01 mmol), DMF (1 mL). The crude was taken directly to the next step without further purification. LCMS (220 nm, 254 nm): t_{R} 2.750 min, purity ≥95%, m/z (ESI): 430.25 [M+H]⁺.

### Synthesis of 5-(2-methyl-6-{N-methyl-2H,3H-thieno[2,3-b][ ],4]dioxine-6-amido}phenyl)thiophene-2-carboxylic acid (RU-NT-423)

According to the general procedure for saponification of methyl esters. Crude from the previous step. Purification using preparative HPLC Method B. Solvent was concentrated under vacuo to give the desired product as a white solid (1.7 mg, 29% yield), quantitative yield assumed from previous step. LCMS (220 nm, 254 nm): t_{R} 2.359 min, purity ≥95%, m/z (ESI): 416.30 [M+H]⁺. ¹H NMR (500 MHz, DMSO-d₆) δ 13.11 (br s, 1H), 7.73 (d, J=3.7 Hz, 1H), 7.34 (d, J=7.2 Hz, 2H), 7.01 (d, J=3.8 Hz, 2H), 6.73 (s, 1H), 4.13-3.86 (m, 4H), 2.92 (s, 3H), 2.20 (s, 3H).

### Synthesis of 5-[2-(3,4-dimethylthiophene-2-amido)-6-methylphenyl]thiophene-2-carboxylic acid (RU-NT-425)

According to the general procedure for saponification of methyl esters. Methyl 5-[2-(3,4-dimethylthiophene-2-amido)-6-methylphenyl]thiophene-2-carboxylate (10 mg, 0.03 mmol). Purification using preparative HPLC Method B. Solvent was concentrated under vacuo to give the desired product as a white solid (9 mg, 81% yield), quantitative yield assumed from previous step. LCMS (220 nm, 254 nm): t_{R} 2.699 min, purity ≥95%, m/z (ESI): 372.25 [M+H]⁺. ¹H NMR (500 MHz, DMSO-d₆) δ 13.12 (br s, 1H), 8.94 (s, 1H), 7.72 (s, 1H), 7.53 (d, J=8.3 Hz, 1H), 7.39 (t, J=7.8 Hz, 1H), 7.25 (d, J=7.8 Hz, 1H), 7.07 (d, J=3.7 Hz, 2H), 3.29 (s, 3H), 2.17 (s, 3H), 2.08 (d, J=1.0 Hz, 3H).

### Synthesis of methyl 5-[2-methyl-6-(N-methyl-3,4-dimethylthiophene-2-amido)phenyl]thiophene-2-carboxylate

According to the general procedure for N-alkylation of amides (Method B). Methyl 5-[2-(3,4-dimethylthiophene-2-amido)-6-methylphenyl]thiophene-2-carboxylate (40 mg, 0.1 mmol), DMF (1 mL). The crude was taken to the next step directly without further purification. LCMS (220 nm, 254 nm): t_{R} 3.077 min, purity ≥95%, m/z (ESI): 400.35 [M+H]⁺.

### Synthesis of 5-[2-methyl-6-(N-methyl-3,4-dimethylthiophene-2-amido)phenyl]thiophene-2-carboxylic acid (RU-NT-419)

According to the general procedure for saponification of methyl esters. Crude from the previous step. Purification using preparative HPLC Method B. Solvent was concentrated under vacuo to give the desired product as a white solid (3.4 mg, 9% yield), quantitative yield assumed from previous step. LCMS (220 nm, 254 nm): t_{R} 2.625 min, purity ≥95%, m/z (ESI): 386.25 [M+H]⁺. ¹H NMR (500 MHz, DMSO-d₆) δ 13.15 (br s, 1H), 7.71 (d, J=3.8 Hz, 1H), 7.34 (s, 2H), 7.26 (s, 1H), 7.09 (s, 1H), 6.79 (s, 1H), 3.06 (s, 1H), 2.13 (s, 3H), 2.04 (s, 3H).

### Synthesis of N-(2-bromo-3-methylphenyl)-5-methyl-1-benzothiophene-2-carboxamide

According to the general procedure E for amide coupling. 5-Methyl-1-benzothiophene-2-carboxylic acid (0.3 g, 1.56 mmol), 2-bromo-3-methylaniline (0.35 g, 1.87 mmol) in DCM (5 mL). Purification using preparative HPLC Method B. Solvent was concentrated under vacuo to give the desired product as beige crystals (75 mg, 13% yield). LCMS (220 nm, 254 nm): t_{R} 3.437 min, purity ≥95%, m/z (EI): 360.15 [M]⁺.

### Synthesis of methyl 5-[2-methyl-6-(5-methyl-1-benzothiophene-2-amido)phenyl]thiophene-2-carboxylate & 5-[2-methyl-6-(5-methyl-1-benzothiophene-2-amido)phenyl]thiophene-2-carboxylic acid (RU-NT-426)

According to the general procedure for Suzuki coupling reactions using Method A, precatalyst C1. N-(2-bromo-3-methylphenyl)-5-methyl-1-benzothiophene-2-carboxamide (70 mg, 0.19 mmol), [5-(methoxycarbonyl)thiophen-2-yl]boronic acid (43.37 mg, 0.23 mmol), dry dioxane (1 mL). Temperature: 100° C. Purification using preparative HPLC Method B. Solvent was concentrated under vacuo to give the desired ester product as an off-white solid (19.4 mg, 24% yield). LCMS of the ester (220 nm, 254 nm): t_{R} 3.333 min, purity ≥95%, m/z (ESI): 422.25 [M+H]⁺ and the acid side product as a pink solid (4 mg, 5% yield). LCMS (220 nm, 254 nm): t_{R} 2.863 min, purity ≥95%, m/z (EI): 408.20 [M+H]⁺. ¹H NMR of the acid (500 MHz, DMSO-d₆) δ 13.05 (s, 1H), 9.94 (s, 1H), 7.89 (s, 1H), 7.87 (s, 1H), 7.71 (s, 1H), 7.69 (d, J=3.9 Hz, 1H), 7.41 (t, J=7.7 Hz, 1H), 7.38-7.23 (m, 3H), 7.08 (d, J=3.8 Hz, 1H), 2.42 (s, 3H), 2.23 (s, 3H).

### Synthesis of methyl 5-[2-methyl-6-(N-methyl-5-methyl-I-benzothiophene-2-amido)phenyl]thiophene-2-carboxylate

According to the general procedure for N-alkylation of amides (Method B). Methyl 5-[2-methyl-6-(5-methyl-1-benzothiophene-2-amido)phenyl]thiophene-2-carboxylate (17 mg, 0.04 mmol), THF (1 mL). The crude was taken directly to the next step without further purification. LCMS (220 nm, 254 nm): t_{R} 3.380 min, purity ≥95%, m/z (ESI): 436.40 [M+H]⁺.

### Synthesis of 5-[2-methyl-6-(N-methyl-5-methyl-1-benzothiophene-2-amido)phenyl]thiophene-2-carboxylic acid (RU-NT-424)

According to the general procedure for saponification of methyl esters. Crude from the previous step. Purification using preparative HPLC Method B. Solvent was concentrated under vacuo to give the desired product as a white solid (2.1 mg, 12% yield), quantitative yield assumed from previous step. LCMS of the ester (220 nm, 254 nm): t_{R} 2.950 min, purity ≥95%, m/z (ESI): 422.20 [M+H]⁺. ¹H NMR (500 MHz, DMSO-d₆) δ 13.14 (br s, 1H), 7.77 (d, J=8.3 Hz, 1H), 7.67 (d, J=3.8 Hz, 1H), 7.61-7.48 (m, 3H), 7.49-7.39 (m, 1H), 7.23 (d, J=8.3 Hz, 1H), 6.90 (s, 1H), 6.85 (d, J=3.8 Hz, 1H), 3.10 (s, 3H), 2.36 (s, 3H), 2.19 (s, 3H).

### Synthesis of N-(2-bromo-3-methylphenyl)-4,5,6,7-tetrahydro-2-benzothiophene-1-carboxamide

According to the general procedure A for amide coupling. Step 1: 4,5,6,7-tetrahydro-2-benzothiophene-1-carboxylic acid (0.3 g, 1.65 mmol), 2 drops DMF, oxalyl chloride (2 mL) at ambient temperature O/N. Step 2: 4,5,6,7-Tetrahydro-2-benzothiophene-1-carbonyl chloride, 2-bromo-3-methylaniline (0.37 g, 1.98 mmol), pyridine (3 mL) at ambient temperature O/N. Purification using preparative HPLC Method B. Solvent was concentrated under vacuo to give the desired product as a brownish solid (82.6 mg, 14% yield). LCMS (220 nm, 254 nm): t_{R} 3.577 min, purity ≥95%, m/z (ESI): 352.20 [M+H]⁺.

### Synthesis of methyl 5-[2-methyl-6-(4,5,6,7-tetrahydro-2-benzothiophene-]-amido)phenyl]thiophene-2-carboxylate

According to the general procedure for Suzuki coupling reactions using Method A, precatalyst C1. N-(2-bromo-3-methylphenyl)-4,5,6,7-tetrahydro-2-benzothiophene-1-carboxamide (80 mg, 0.23 mmol), [5-(methoxycarbonyl)thiophen-2-yl]boronic acid (50.98 mg, 0.27 mmol), dry dioxane (4 mL). Temperature: 100° C. Purification using preparative HPLC Method B. Solvent was concentrated under vacuo to give the desired product as a beige solid (15.2 mg, 16% yield). LCMS (220 nm, 254 nm): t_{R} 3.443 min, purity ≥95%, m/z (ESI): 412.35 [M+H]⁺. ¹H NMR (500 MHz, DMSO-d₆) δ 8.61 (s, 1H), 7.87 (d, J=3.8 Hz, 1H), 7.67 (d, J=8.0 Hz, 1H), 7.40 (t, J=7.9 Hz, 1H), 7.27 (s, 1H), 7.23 (d, J=7.6 Hz, 1H), 7.15 (d, J=3.8 Hz, 1H), 3.83 (s, 3H), 2.61 (t, J=6.3 Hz, 2H), 2.16 (s, 3H), 1.75-1.44 (m, 4H), 1.19 (d, J=46.5 Hz, 2H).

### Synthesis of 5-[2-methyl-6-(4,5,6,7-tetrahydro-2-benzothiophene-1-amido)phenyl]thiophene-2-carboxylic acid (RU-NT-429)

According to the general procedure for saponification of methyl esters. Methyl 5-[2-methyl-6-(4,5,6,7-tetrahydro-2-benzothiophene-1-amido)phenyl]thiophene-2-carboxylate (5 mg, 0.01 mmol). Purification using preparative HPLC Method B. Solvent was concentrated under vacuo to give the desired ester product as a white solid (3 mg, 62% yield). LCMS (220 nm, 254 nm): t_{R} 2.905 min, purity ≥95%, m/z (ESI): 398.40 [M+H]⁺. ¹H NMR (500 MHz, DMSO-d₆) δ 13.29 (br s, 1H), 8.63 (s, 1H), 7.89 (d, J=3.8 Hz, 1H), 7.83 (d, J=8.1 Hz, 1H), 7.50 (s, 1H), 7.39 (s, 1H), 7.33 (d, J=7.6 Hz, 1H), 7.23 (d, J=3.7 Hz, 1H), 2.72 (t, J=6.3 Hz, 2H), 2.55 (t, J=6.1 Hz, 2H), 2.27 (s, 3H), 2.17-2.05 (m, 1H), 1.82-1.53 (m, 3H), 1.51-1.23 (m, 2H).

### Synthesis of methyl 5-[2-methyl-6-(N-methyl-4,5,6,7-tetrahydro-2-benzothiophene-]-amido)phenyl]thiophene-2-carboxylate

According to the general procedure for N-alkylation of amides (Method B). Methyl 5-[2-methyl-6-(4,5,6,7-tetrahydro-2-benzothiophene-1-amido)phenyl]thiophene-2-carboxylate (10 mg, 0.02 mmol), DMF (1 mL). The crude was taken directly to the next step after extraction according to the general procedure. LCMS (220 nm, 254 nm): t_{R} 3.296 min, purity ≥95%, m/z (ESI): 426.30 [M+H]⁺.

### Synthesis of 5-[2-methyl-6-(N-methyl-4,5,6,7-tetrahydro-2-benzothiophene-1-amido)phenyl]thiophene-2-carboxylic acid (RU-NT-430)

According to the general procedure for saponification of methyl esters. Methyl 5-[2-methyl-6-(N-methyl-4,5,6,7-tetrahydro-2-benzothiophene-1-amido)phenyl]thiophene-2-carboxylate (35 mg, 0.02 mmol). Purification using preparative HPLC Method B. Solvent was concentrated under vacuo to give the desired ester product as a white solid (20.5 mg, 13% yield). LCMS (220 nm, 254 nm): t_{R} 2.820 min, purity ≥95%, m/z (ESI): 412.15 [M+H]⁺. ¹H NMR (500 MHz, DMSO-d₆) δ 13.13 (br s, 1H), 7.68 (d, J=3.7 Hz, 1H), 7.41 (t, J=7.7 Hz, 1H), 7.40-7.35 (m, 1H), 7.32 (dd, J=7.7, 1.4 Hz, 1H), 7.07 (s, 1H), 6.76 (s, 1H), 3.08 (s, 3H), 2.75 (s, 1H), 2.57 (s, 2H), 2.46-2.35 (m, 1H), 2.13 (s, 3H), 1.59 (d, J=30.8 Hz, 4H).

### Synthesis of N-(2-bromo-3-methylphenyl)-N-(cyanomethyl)-5-methylthiophene-2-carboxamide

According to the general procedure for N-alkylation of amides (Method A). N-(2-bromo-3-methylphenyl)-5-methylthiophene-2-carboxamide (56 mg, 0.18 mmol), acetonitrile (4 mL), 2-bromoacetonitrile (108.27 mg, 0.90 mmol, 5 eq). Purification using preparative HPLC Method B. Solvent was concentrated under vacuo to give the desired product as a brownish solid (31.6 mg, 50% yield). LCMS (220 nm, 254 nm): t_{R} 2.724 min, purity ≥95%, m/z (ESI): 349.20 [M+H]⁺.

### Synthesis of methyl 5-{2-[N-(cyanomethyl)-5-methylthiophene-2-amido]-6-methylphenyl}thiophene-2-carboxylate & 5-{2-[N-(cyanomethyl)-5-methylthiophene-2-amido]-6-methylphenyl}thiophene-2-carboxylic acid (RU-NT-431)

According to the general procedure for Suzuki coupling reactions using Method A, precatalyst C1. N-(2-bromo-3-methylphenyl)-N-(cyanomethyl)-5-methylthiophene-2-carboxamide (34 mg, 0.1 mmol), [5-(methoxycarbonyl)thiophen-2-yl]boronic acid (21.73 mg, 0.12 mmol), 1 mL dry dioxane. Purification using preparative HPLC Method B. Solvent was concentrated under vacuo to give the desired ester product as a beige solid (5.5 mg, 14% yield). LCMS (220 nm, 254 nm): t_{R} 2.909 min, purity ≥95%, m/z (ESI): 411.25 [M+H]⁺ and the acid side product as a beige solid (6.5 mg, 17% yield). LCMS (220 nm, 254 nm): t_{R} 2.183 min, purity ≥95%, m/z (ESI): 398.20 [M+H]⁺. ¹H NMR (500 MHz, DMSO-d₆) δ 13.17 (br s, 1H), 7.69 (d, J=3.7 Hz, 1H), 7.63-7.19 (m, 2H), 6.98 (s, 1H), 6.87 (d, J=3.7 Hz, 1H), 6.64 (dd, J=3.8, 1.1 Hz, 1H), 6.38 (d, J=3.8 Hz, 1H), 4.38 (d, J=16.2 Hz, 1H), 2.35 (s, 3H), 2.18 (s, 3H).

### Synthesis of 2-bromo-3-methyl-N-(thiophen-2-yl)benzamide

According to the general procedure A for amide coupling. Thiophen-2-amine (0.1 g, 1.01 mmol), commercially available 2-bromo-3-methylbenzoyl chloride (0.24 g, 1.01 mmol), DCM (5 mL). Rtp, O/N. Purification using preparative HPLC Method B. Solvent was concentrated under vacuo to give the desired ester product as a beige solid (0.1 g, 34% yield). LCMS (220 nm, 254 nm): t_{R} 2.371 min, purity ≥95%, m/z (ESI): 298.30 [M+H]⁺.

### Synthesis of methyl 5-{2-methyl-6-[(thiophen-2-yl)carbamoyl]phenyl}thiophene-2-carboxylate

According to the general procedure for Suzuki coupling reactions using Method A, precatalyst C1. 2-Bromo-3-methyl-N-(thiophen-2-yl)benzamide (70 mg, 0.24 mmol), [5-(methoxycarbonyl)thiophen-2-yl]boronic acid (48.35 mg, 0.26 mmol), dry dioxane (3 mL). Purification using preparative HPLC Method B. Solvent was concentrated under vacuo to give the desired product as a beige solid (50 mg, 59% yield). LCMS (220 nm, 254 nm): t_{R} 2.597 min, purity ≥95%, m/z (ESI): 358.25 [M+H]⁺.

### Synthesis of 5-{2-methyl-6-[(thiophen-2-yl)carbamoyl]phenyl}thiophene-2-carboxylic acid (RU-NT-432)

According to the general procedure for saponification of methyl esters. Methyl 5-{2-methyl-6-[(thiophen-2-yl)carbamoyl]phenyl}thiophene-2-carboxylate (20 mg, 0.06 mmol). Purification using preparative HPLC Method B. Solvent was concentrated under vacuo to give the desired product as a white solid (7 mg, 36% yield). LCMS (220 nm, 254 nm): t_{R} 2.172 min, purity ≥95%, m/z (ESI): 344.30 [M+H]⁺. ¹H NMR (500 MHz, DMSO-d₆) δ 13.09 (br s, 1H), 11.44 (s, 1H), 7.65 (d, J=3.8 Hz, 1H), 7.53-7.45 (m, 2H), 7.44-7.34 (m, 1H), 7.04 (d, J=3.8 Hz, 1H), 6.93 (d, J=5.4 Hz, 1H), 6.82 (dd, J=5.5, 3.7 Hz, 1H), 6.64 (d, J=3.5 Hz, 1H), 2.23 (s, 3H).

### Synthesis of methyl 5-{2-methyl-6-[N-(prop-2-yn-1-yl)-5-methylthiophene-2-amido]phenyl}thiophene-2-carboxylate

According to the general procedure for N-alkylation of amides (Method B). Methyl 5-[2-methyl-6-(5-methylthiophene-2-amido)phenyl]thiophene-2-carboxylate (60 mg, 0.16 mmol), DMF (1 mL), excess propargyl bromide. The crude was taken directly to the next step. LCMS (220 nm, 254 nm): t_{R} 3.063 min, purity ≥95%, m/z (ESI): 410.30 [M+H]⁺.

### Synthesis of 5-{2-methyl-6-[N-(prop-2-yn-1-yl)-5-methylthiophene-2-amido]phenyl}thiophene-2-carboxylic acid (RU-NT-433)

According to the general procedure for saponification of methyl esters. Methyl 5-{2-methyl-6-[N-(prop-2-yn-1-yl)-5-methylthiophene-2-amido]phenyl}thiophene-2-carboxylate. Purification using preparative HPLC Method B. Solvent was concentrated under vacuo to give the desired product as a white solid (2.16 mg, 11% yield), assume quantitative yield from previous step. LCMS (220 nm, 254 nm): t_{R} 2.653 min, purity ≥95%, m/z (ESI): 396.15 [M+H]⁺. H NMR (500 MHz, DMSO-d₆) δ 13.17 (s, 1H), 7.67 (d, J=3.7 Hz, 1H), 7.51 (dt, J=15.3, 7.7 Hz, 1H), 7.45-6.93 (m, 4H), 6.66 (s, 1H), 4.67 (d, J=17.4 Hz, 2H), 3.20 (s, 1H), 2.36 (s, 3H), 2.20 (s, 3H).

### Synthesis of methyl 5-{2-methyl-6-[N-(propan-2-yl)-5-methylthiophene-2-amido]phenyl}thiophene-2-carboxylate

According to the general procedure for N-alkylation of amides (Method B). Methyl 5-[2-methyl-6-(5-methylthiophene-2-amido)phenyl]thiophene-2-carboxylate (60 mg, 0.16 mmol), DMF (1 mL), 2-bromopropane (5 eq). The layers of the resulting biphasic mixture was separated and aqueous layer was extracted with EtOAc (3×). The crude (20 mg, 30% yield) was taken directly to the next step without further purification. LCMS (220 nm, 254 nm): t_{R} 3.253 min, purity ≥95%, m/z (ESI): 414.35 [M+H]⁺.

### Synthesis of 5-{2-methyl-6-[N-(propan-2-yl)-5-methylthiophene-2-amido]phenyl}thiophene-2-carboxylic acid (RU-NT-434)

According to the general procedure for saponification of methyl esters. Methyl 5-{2-methyl-6-[N-(propan-2-yl)-5-methylthiophene-2-amido]phenyl}thiophene-2-carboxylate (20 mg, 0.05 mmol). Purification using preparative HPLC Method B. Solvent was concentrated under vacuo to give the desired product as a white solid (5.59 mg, 29% yield). LCMS (220 nm, 254 nm): t_{R} 2.811 min, purity ≥95%, m/z (ESI): 400.25 [M+H]⁺. ¹H NMR (500 MHz, DMSO-d₆) δ 13.16 (br s, 1H), 7.69 (d, J=3.8 Hz, 1H), 7.50 (dt, J=7.5, 1.2 Hz, 1H), 7.45 (t, J=7.7 Hz, 1H), 7.23 (dd, J=8.0, 1.4 Hz, 1H), 6.91 (d, J=3.7 Hz, 1H), 6.60 (dd, J=3.8, 1.2 Hz, 1H), 6.23 (d, J=3.8 Hz, 1H), 4.06 (p, J=6.8 Hz, 1H), 2.34 (d, J=1.0 Hz, 3H), 2.20 (s, 3H), 1.01 (d, J=6.7 Hz, 3H), 0.93 (d, J=6.8 Hz, 3H).

### Synthesis of methyl 5-[2-methyl-6-(5-methylthiophene-2-amido)phenyl]thiophene-2-carboxylate

According to the general procedure for Suzuki coupling reactions using Method A, precatalyst C1. Methyl [5-(methoxycarbonyl)thiophen-2-yl]boronic acid (160 mg, 0.89 mmol), N-(2-bromo-3-methylphenyl)-5-methylthiophene-2-carboxamide (0.32 g, 0.81 mmol), dry dioxane (10 mL). Temperature: 130° C. Purification using preparative HPLC Method B. Solvent was concentrated under vacuo to give the desired product as a white solid (23.6 mg, 8% yield). LCMS (220 nm, 254 nm): t_{R} 3.041 min, purity ≥95%, m/z (ESI): 372.15 [M+H]⁺. ¹H NMR (500 MHz, DMSO-d₆) δ 9.53 (s, 1H), 7.78 (d, J=3.8 Hz, 1H), 7.42 (d, J=3.7 Hz, 1H), 7.40-7.21 (m, 2H), 7.09 (d, J=3.8 Hz, 1H), 6.81 (dd, J=3.7, 1.2 Hz, 1H), 3.80 (s, 3H), 2.44 (s, 3H), 2.20 (s, 3H).

### Synthesis of methyl 5-[2-(N-cyclopropyl-5-methylthiophene-2-amido)-6-methylphenyl]thiophene-2-carboxylate

A 10 mL MW Biotage vial was charged with methyl 5-[2-methyl-6-(5-methylthiophene-2-amido)phenyl]thiophene-2-carboxylate (62 mg 0.17 mmol), Cu(OAc)₂ (30.32 mg, 0.17 mmol) and Cs₂CO₃ (27.19 mg, 0.08 mmol). Pyridine (3.0 eq.) cyclopropylboronic acid (28.67 mg, 0.33 mmol) and toluene (1 mL) were added. The tube was sealed, degassed for 3 mins under N₂ and heated to 110° C. overnight. After cooling to room temperature, dichloromethane wad added. The organic phase was washed twice with water and brine three times. Organic layers were gathered, dried over MgSO₄, filtered and concentrated under vacuum. Purification using preparative HPLC Method B. Solvent was concentrated under vacuo to give the desired product as a clear oil (12 mg, 17% yield). LCMS (220 nm, 254 nm): t_{R} 3.089 min, purity ≥95%, m/z (ESI): 412.30 [M+H]⁺.

### Synthesis of 5-[2-(N-cyclopropyl-5-methylthiophene-2-amido)-6-methylphenyl]thiophene-2-carboxylic acid (RU-NT-435)

According to the general procedure for saponification of methyl esters. Methyl 5-[2-(N-cyclopropyl-5-methylthiophene-2-amido)-6-methylphenyl]thiophene-2-carboxylate (8 mg, 0.02 mmol). Purification using preparative HPLC Method B. Solvent was concentrated under vacuo to give the desired product as a white solid (1.2 mg, 16% yield). LCMS (220 nm, 254 nm): t_{R} 2.665 min, purity ≥95%, m/z (ESI): 398.25 [M+H]⁺. ¹H NMR (500 MHz, DMSO-d₆) δ 13.15 (br s, 1H), 7.69 (d, J=3.7 Hz, 1H), 7.43 (t, J=7.5 Hz, 1H), 7.13 (d, J=7.5 Hz, 1H), 6.83-6.18 (m, 1H), 6.59 (m, 3H), 5.32 (s, 1H), 2.50 (s, 3H), 2.27 (s, 3H), 1.50-1.07 (m, 4H).

### Synthesis of N-[2-bromo-3-(trifluoromethyl)phenyl]-5-methylthiophene-2-carboxamide

According to the general procedure A for amide coupling. 2-Bromo-3-(trifluoromethyl)aniline (0.1 g, 0.42 mmol), commercially available 5-methylthiophene-2-carbonyl chloride (0.07 g, 0.42 mmol). DCM (1 mL). Rtp, O/N. LCMS (220 nm, 254 nm): t_{R} 2.595 min, purity ≥95%, m/z (ESI): 387.45 [M+CH₃CN+Na, 100%]⁺.

### Synthesis of 5-[2-(5-methylthiophene-2-amido)-6-(trifluoromethyl)phenyl]thiophene-2-carboxylic acid (RU-NT-436)

According to the general procedure for Suzuki coupling reactions using Method A, precatalyst C1. N-[2-bromo-3-(trifluoromethyl)phenyl]-5-methylthiophene-2-carboxamide (55 mg, 0.15 mmol), [5-(methoxycarbonyl)thiophen-2-yl]boronic acid (36 mg, 0.2 mmol), dry dioxane (1.5 mL). Temperature: 120° C. Purification using preparative HPLC Method B. Solvent was concentrated under vacuo to give the desired product as a yellow oil (50 mg, 81% yield). LCMS (220 nm, 254 nm): t_{R} 2.619 min, purity ≥95%, m/z (ESI): 412.40 [M+H]⁺. ¹H NMR (500 MHz, DMSO-d₆) δ 13.22 (br s, 1H), 9.61 (s, 1H), 7.83 (t, J=7.4 Hz, 2H), 7.74 (t, J=8.0 Hz, 1H), 7.39 (d, J=3.7 Hz, 1H), 7.08 (d, J=3.8 Hz, 1H), 6.83 (d, J=3.7 Hz, 1H), 2.45 (s, 3H). ¹⁹F NMR (471 MHz, DMSO-d₆) δ-56.31 Hz.

### Synthesis of methyl 5-[2-(N-methyl-5-methylthiophene-2-amido)-6-(trifluoromethyl)phenyl]thiophene-2-carboxylate

According to the general procedure for N-alkylation of amides (Method B). 5-[2-(5-Methylthiophene-2-amido)-6-(trifluoromethyl)phenyl]thiophene-2-carboxylic acid (50 mg, 0.1 mmol), DMF (1 mL). Purification using preparative HPLC Method B. Solvent was concentrated under vacuo to give the desired product as a colorless oil (12.5 mg, 30% yield). LCMS (220 nm, 254 nm): t_{R} 2.992 min, purity ≥95%, m/z (ESI): 440.25 [M+H]⁺.

### Synthesis of 5-[2-(N-methyl-5-methylthiophene-2-amido)-6-(trifluoromethyl)phenyl]thiophene-2-carboxylic acid (RU-NT-437)

According to the general procedure for saponification of methyl esters. Methyl 5-[2-(N-methyl-5-methylthiophene-2-amido)-6-(trifluoromethyl)phenyl]thiophene-2-carboxylate (12 mg, 0.03 mmol). Purification using preparative HPLC Method B. Solvent was concentrated under vacuo to give the desired product as a white solid (5 mg, 43% yield). LCMS (220 nm, 254 nm): t_{R} 2.579 min, purity ≥95%, m/z (ESI): 426.20 [M+H]⁺. ¹H NMR (500 MHz, DMSO-d₆) δ 13.31 (br s, 1H), 8.03 (d, J=8.0 Hz, 1H), 7.96-7.78 (m, 1H), 7.65 (d, J=3.8 Hz, 1H), 6.85 (s, 1H), 6.66 (s, 1H), 6.41 (s, 1H), 3.05 (s, 3H), 2.36 (s, 3H). ¹⁹F NMR (471 MHz, DMSO-d₆) δ-56.52 Hz.

### Synthesis of N-(2-bromo-3-methylphenyl)-5-methylthiophene-2-carboxamide

According to the general procedure A for amide coupling. 2-Bromo-3-methylaniline (0.26 g, 1.4 mmol), commercially available 5-methylthiophene-2-carbonyl chloride (0.25 g, 1.28 mmol), THF (10 mL). Rtp O/N. The crude mixture was dissolved in MeOH, fractionated and purification carried out using preparative HPLC Method B. Solvent was concentrated under vacuo to give the desired product as a yellow solid (0.37 g, 93% yield). LCMS (220 nm, 254 nm): t_{R} 3.056 min, purity ≥95%, m/z (ESI): 310.10 [M+H]⁺. ¹H NMR (500 MHz, DMSO-d₆) δ 9.89 (s, 1H), 7.81 (d, J=3.7 Hz, 1H), 7.48-7.14 (m, 3H), 7.02-6.84 (m, 1H), 2.50 (s, 3H), 2.41 (s, 3H).

### Synthesis of N-[2-(5-cyanothiophen-2-yl)-3-methylphenyl]-5-methylthiophene-2-carboxamide

According to the general procedure for Suzuki coupling reactions using Method A, precatalyst C1. N-(2-bromo-3-methylphenyl)-5-methylthiophene-2-carboxamide (50 mg, 0.16 mmol), (5-cyanothiophen-2-yl)boronic acid (73.96 mg, 0.48 mmol), dry dioxane (2 mL), aq. 1M K₃PO₄(1 eq). Temperature: 150° C. Purification using preparative HPLC Method B. Solvent was concentrated under vacuo to give the desired product as a brownish oil (40 mg, 73% yield). LCMS (220 nm, 254 nm): t_{R} 2.787 min, purity ≥95%, m/z (ESI): 339.20 [M+H]⁺.

### Synthesis of N-[2-(5-cyanothiophen-2-yl)-3-methylphenyl]-N,5-dimethylthiophene-2-carboxamide

According to the general procedure for N-alkylation of amides (Method B). N-[2-(5-cyanothiophen-2-yl)-3-methylphenyl]-5-methylthiophene-2-carboxamide (40 mg, 0.12 mmol), DMF (1 mL). Purification using preparative HPLC Method B. Solvent was concentrated under vacuo to give the desired product as white crystals (40 mg, 96% yield). LCMS (220 nm, 254 nm): t_{R} 2.867 min, purity ≥95%, m/z (ESI): 353.25 [M+H]⁺.

### Synthesis of N,5-dimethyl-N-{3-methyl-2-[5-(]H-1,2,3,4-tetrazol-5-yl)thiophen-2-yl]phenyl}thiophene-2-carboxamide (RU-NT-438)

According to the general procedure for tetrazole formation. N-[2-(5-Cyanothiophen-2-yl)-3-methylphenyl]-N,5-dimethylthiophene-2-carboxamide (40 mg, 0.11 mol). Temperature: 50° C. Purification using preparative HPLC Method B. Solvent was concentrated under vacuo to give the desired product as a white solid (6 mg, 13% yield). LCMS (220 nm, 254 nm): t_{R} 2.627 min, purity ≥95%, m/z (ESI): 396.15 [M+H]⁺. ¹H NMR (500 MHz, DMSO-d₆) δ 17.00 (br s, 1H), 7.76 (d, J=3.7 Hz, 1H), 7.68-7.42 (m, 1H), 7.42-7.25 (m, 2H), 7.00 (d, J=3.7 Hz, 1H), 6.64 (d, J=3.8 Hz, 1H), 6.39 (d, J=3.7 Hz, 1H), 3.05 (s, 3H), 2.36 (s, 3H), 2.23 (s, 3H).

### Synthesis of N-(2-bromo-3-methylphenyl)-3-methylthiophene-2-carboxamide

According to the general procedure A for amide coupling. 3-Methylthiophene-2-carboxylic acid (0.3 g, 1.87 mmol), thionyl chloride (1.6 mL), DMF (1 mL). Time: 3 hours. Step 2: 2-bromo-3-methylaniline (0.35 g, 1.87 mmol), DCM (2 mL) and excess TEA (2 eq). Temperature: 40° C. overnight. Purification using preparative HPLC Method B. Solvent was concentrated under vacuo to give the desired product as a white solid (0.4 g, 69% yield, 91% pure). LCMS (220 nm, 254 nm): t_{R} 3.252 min, purity ≥95%, m/z (ESI): 310.15 [M+H]⁺.

### Synthesis of N-[2-(5-cyanothiophen-2-yl)-3-methylphenyl]-3-methylthiophene-2-carboxamide

According to the general procedure for Suzuki coupling reactions using Method A, precatalyst C1. N-(2-bromo-3-methylphenyl)-3-methylthiophene-2-carboxamide (50 mg, 0.16 mmol), (5-cyanothiophen-2-yl)boronic acid (74 mg, 0.48 mmol), dry dioxane (2 mL), aq. 1M K₃PO₄(1 eq). Temperature: 150° C. Purification using preparative HPLC Method B. Solvent was concentrated under vacuo to give the desired product as white crystals (28.5 mg, 52% yield). LCMS (220 nm, 254 nm): t_{R} 2.881 min, purity ≥95%, m/z (ESI): 339.10 [M+H]⁺. ¹H NMR (500 MHz, DMSO-d₆) δ 9.17 (s, 1H), 8.01 (d, J=3.8 Hz, 1H), 7.59 (d, J=5.0 Hz, 1H), 7.51-7.32 (m, 2H), 7.29 (d, J=7.2 Hz, 1H), 7.19 (d, J=3.8 Hz, 1H), 6.94 (d, J=5.0 Hz, 1H), 2.20 (s, 3H), 2.18 (s, 3H).

### Synthesis of N-[2-(5-cyanothiophen-2-yl)-3-methylphenyl]-N, 3-dimethylthiophene-2-carboxamide

According to the general procedure for N-alkylation of amides (Method B). N-[2-(5-cyanothiophen-2-yl)-3-methylphenyl]-3-methylthiophene-2-carboxamide (25 mg, 0.07 mmol), DMF (1 mL). Purification using preparative HPLC Method B. Solvent was concentrated under vacuo to give the desired product as a beige solid (25 mg, 96% yield). LCMS (220 nm, 254 nm): t_{R} 2.836 min, purity ≥95%, m/z (ESI): 353.05 [M+H]⁺.

### Synthesis of N, 3-dimethyl-N-{3-methyl-2-[5-(]H-1,2,3,4-tetrazol-5-yl)thiophen-2-yl]phenyl}thiophene-2-carboxamide (RU-NT-439)

According to the general procedure for tetrazole formation. N-[2-(5-cyanothiophen-2-yl)-3-methylphenyl]-N,3-dimethylthiophene-2-carboxamide (20 mg, 0.06 mmol). Temperature: 50° C. Purification using preparative HPLC Method B. Solvent was concentrated under vacuo to give the desired product as a white solid (11.3 mg, 50% yield). LCMS (220 nm, 254 nm): t_{R} 2.431 min, purity ≥95%, m/z (ESI): 396.10 [M+H]⁺. ¹H NMR (500 MHz, DMSO-d₆) δ 16.82 (br s, 1H), 7.79 (d, J=3.6 Hz, 1H), 7.47-7.35 (m, 3H), 7.31 (d, J=7.2 Hz, 1H), 6.92 (s, 1H), 6.82 (d, J=5.0 Hz, 1H), 3.10 (s, 3H), 2.18 (s, 6H).

### Synthesis of methyl 5-{[3-methyl-2-(thiophen-2-yl)phenyl]carbamoyl}thiophene-2-carboxylate & 5-{[3-methyl-2-(thiophen-2-yl)phenyl]carbamoyl}thiophene-2-carboxylic acid (RU-NT-440)

According to the general procedure for Suzuki coupling reactions using Method A, precatalyst C1. Methyl 5-[(2-bromo-3-methylphenyl)carbamoyl]thiophene-2-carboxylate (100 mg, 0.28 mmol), (thiophen-2-yl)boronic acid (108.36 mg, 0.85 mmol), dry dioxane (3 mL), aq. 1M K₃PO₄(1 eq). Temperature: 150° C. Purification using preparative HPLC Method B. Solvent was concentrated under vacuo to give the desired ester product as white crystals (45.5 mg, 45% yield). LCMS (220 nm, 254 nm): t_{R} 3.041 min, purity ≥95%, m/z (ESI): 358.05 [M+H]⁺ and the acid side product: white solid (13.1 mg, 13% yield). LCMS (220 nm, 254 nm): t_{R} 2.613 min, purity ≥95%, m/z (ESI): 344.05 [M+H]⁺. ¹H NMR (500 MHz, DMSO-d₆) δ 13.43 (br s, 1H), 9.69 (s, 1H), 7.68 (d, J=4.0 Hz, 1H), 7.64-7.58 (m, 1H), 7.56 (d, J=4.0 Hz, 1H), 7.36 (d, J=4.6 Hz, 2H), 7.28 (t, J=4.5 Hz, 1H), 7.11 (dd, J=5.2, 3.5 Hz, 1H), 7.03 (dd, J=3.5, 1.2 Hz, 1H), 2.20 (s, 3H), 2.07 (s, 3H).

### Synthesis of methyl 5-{methyl[3-methyl-2-(thiophen-2-yl)phenyl]carbamoyl}thiophene-2-carboxylate

According to the general procedure for N-alkylation of amides (Method B). Methyl 5-{[3-methyl-2-(thiophen-2-yl)phenyl]carbamoyl}thiophene-2-carboxylate (30 mg, 0.08 mmol), DMF (1 mL). Purification using preparative HPLC Method B. Solvent was concentrated under vacuo to give the desired ester product as a white solid (17.1 mg, 55% yield). LCMS (220 nm, 254 nm): t_{R} 2.915 min, purity ≥95%, m/z (ESI): 392.10 [M+H]⁺. ¹H NMR (500 MHz, DMSO-d₆) δ 7.61 (d, J=5.1 Hz, 1H), 7.55 (d, J=4.0 Hz, 1H), 7.52-7.45 (m, 2H), 7.41 (dd, J=6.2, 3.1 Hz, 1H), 7.08 (dd, J=5.1, 3.6 Hz, 1H), 6.75 (d, J=3.4 Hz, 1H), 6.57 (d, J=4.1 Hz, 1H), 3.78 (s, 3H), 3.08 (s, 3H), 2.15 (s, 3H).

### Synthesis of 5-{methyl[3-methyl-2-(thiophen-2-yl)phenyl]carbamoyl}thiophene-2-carboxylic acid (RU-NT-441)

According to the general procedure for saponification of methyl esters. Methyl 5-{methyl[3-methyl-2-(thiophen-2-yl)phenyl]carbamoyl}thiophene-2-carboxylate (15 mg, 0.04 mmol). Purification using preparative HPLC Method B. Solvent was concentrated under vacuo to give the desired ester product as a white solid (5.9 mg, 41% yield). LCMS (220 nm, 254 nm): t_{R} 2.485 min, purity ≥95%, m/z (ESI): 358.05 [M+H]⁺. ¹H NMR (500 MHz, DMSO-d₆) δ 13.37 (br s, 1H), 8.00 (br s, 2H), 7.68 (d, J=3.7 Hz, 1H), 7.58-7.27 (m, 4H), 6.77 (d, J=3.7 Hz, 1H), 6.62 (d, J=4.1 Hz, 1H), 3.08 (s, 3H), 2.17 (s, 3H).

### Synthesis of methyl 5-{[2-(5-cyanothiophen-2-yl)-3-methylphenyl]carbamoyl}thiophene-2-carboxylate

According to the general procedure for Suzuki coupling reactions using Method A, precatalyst C1. Methyl 5-[(2-bromo-3-methylphenyl)carbamoyl]thiophene-2-carboxylate (0.5 g, 4.23 mmol), (5-cyanothiophen-2-yl)boronic acid (0.647 g, 4.23 mmol, dry dioxane (14 mL), aq. 1M K₃PO₄(1 eq). Temperature: 150° C. Purification using preparative HPLC Method B. Solvent was concentrated under vacuo to give the desired ester product as an off-white solid (0.121 g, 22% yield). LCMS (220 nm, 254 nm): t_{R} 2.709 min, purity ≥95%, m/z (ESI): 382.15 [M+H]⁺.

### Synthesis of methyl 5-{[2-(5-cyanothiophen-2-yl)-3-methylphenyl](methyl)carbamoyl}thiophene-2-carboxylate

According to the general procedure for N-alkylation of amides (Method B). Methyl 5-{[2-(5-cyanothiophen-2-yl)-3-methylphenyl]carbamoyl}thiophene-2-carboxylate (60 mg, 0.16 mmol), DMF (1 mL). Purification using preparative HPLC Method B. Solvent was concentrated under vacuo to give the desired product as a colorless oil (49.5 mg, 80% yield). LCMS (220 nm, 254 nm): t_{R} 2.767 min, purity ≥95%, m/z (ESI): 438.10 [M+CH₃CN, 100%]⁺.

### Synthesis of 5-{[2-(5-carbamoylthiophen-2-yl)-3-methylphenyl](methyl)carbamoyl}thiophene-2-carboxylic acid (RU-NT-442)

According to the general procedure for saponification of methyl esters. Methyl 5-{[2-(5-cyanothiophen-2-yl)-3-methylphenyl](methyl)carbamoyl}thiophene-2-carboxylate (8 mg, 0.02 mmol), 50° C. O/N. Purification using preparative HPLC Method B. Solvent was concentrated under vacuo to give the desired product as a white solid (5 mg, 62% yield). LCMS (220 nm, 254 nm): t_{R} 1.965 min, purity ≥95%, m/z (ESI): 401.05 [M+H]⁺. ¹H NMR (500 MHz, DMSO-d₆) δ 13.37 (br s, 1H), 8.00 (br s, 2H), 7.68 (d, J=3.7 Hz, 1H), 7.58-7.27 (m, 4H), 6.77 (d, J=3.7 Hz, 1H), 6.62 (d, J=4.1 Hz, 1H), 3.08 (s, 3H), 2.17 (s, 3H).

### Synthesis of methyl 5-[methyl({3-methyl-2-[5-(]H-1,2,3,4-tetrazol-5-yl)thiophen-2-yl]phenyl})carbamoyl]thiophene-2-carboxylate (RU-NT-443)

According to the general procedure for tetrazole formation. Methyl 5-{[2-(5-cyanothiophen-2-yl)-3-methylphenyl](methyl)carbamoyl}thiophene-2-carboxylate (40 mg, 0.1 mmol). Temperature: 50° C. Purification using preparative HPLC Method B. Solvent was concentrated under vacuo to give the desired product as a white solid (19.1 mg, 43% yield). LCMS (220 nm, 254 nm): t_{R} 2.423 min, purity ≥95%, m/z (ESI): 440.05 [M+H]⁺. ¹H NMR (500 MHz, DMSO-d₆) δ 17.01 (br s, 1H), 7.75 (d, J=3.7 Hz, 1H), 7.58 (d, J=4.1 Hz, 1H), 7.54 (d, J=4.7 Hz, 2H), 7.47 (t, J=4.6 Hz, 1H), 6.95 (d, J=3.8 Hz, 1H), 6.62 (d, J=4.1 Hz, 1H), 3.79 (s, 3H), 3.14 (s, 3H), 2.22 (s, 3H).

### Synthesis of 5-[methyl({3-methyl-2-[5-(]H-1,2,3,4-tetrazol-5-yl)thiophen-2-yl]phenyl})carbamoyl]thiophene-2-carboxylic acid (RU-NT-444)

According to the general procedure for saponification of methyl esters. Methyl 5-[methyl({3-methyl-2-[5-(1H-1,2,3,4-tetrazol-5-yl)thiophen-2-yl]phenyl})carbamoyl]thiophene-2-carboxylate (13.2 mg, 0.03 mmol). Purification using preparative HPLC Method B. Solvent was concentrated under vacuo to give the desired product as a white solid (10.1 mg, 79% yield). LCMS (220 nm, 254 nm): t_{R} 2.156 min, purity ≥95%, m/z (ESI): 426.10 [M+H]⁺. ¹H NMR (500 MHz, DMSO-d₆) δ 16.94 (br s, 1H), 13.39 (br s, 1H), 7.76 (d, J=3.7 Hz, 1H), 7.54 (d, J=4.7 Hz, 1H), 7.49 (d, J=4.0 Hz, 1H), 7.45 (t, J=4.7 Hz, 1H), 6.96 (d, J=3.7 Hz, 1H), 6.65 (d, J=4.0 Hz, 1H), 3.13 (s, 3H), 2.23 (s, 3H).

### Synthesis of N-(2-bromo-3-methylphenyl)-3-methyl-1-benzothiophene-2-carboxamide

According to the general procedure B for amide coupling. 3-Methyl-1-benzothiophene-2-carboxylic acid (0.3 g, 1.56 mmol), 2-bromo-3-methylaniline, DCM (3 mL). Temperature: 60° C. Purification using preparative HPLC Method B. Solvent was concentrated under vacuo to give the desired product as white crystals (26.1 mg, 5% yield). LCMS (220 nm, 254 nm): t_{R} 3.541 min, purity ≥95%, m/z (ESI): 362.10 [M+H]⁺.

### Synthesis of N-[2-(5-cyanothiophen-2-yl)-3-methylphenyl]-3-methyl-1-benzothiophene-2-carboxamide

According to the general procedure for Suzuki coupling reactions using Method A, precatalyst C1. N-(2-bromo-3-methylphenyl)-3-methyl-1-benzothiophene-2-carboxamide (150 mg, 0.42 mmol), (5-cyanothiophen-2-yl)boronic acid (191 mg, 1.25 mmol), dry dioxane (4 mL), aq. 1M K₃PO₄(1 eq). Temperature:150° C. Purification using preparative HPLC Method B. Solvent was concentrated under vacuo to give the desired product as white solid (20 mg, 12% yield). LCMS (220 nm, 254 nm): t_{R} 3.245 min, purity ≥95%, m/z (ESI): 389.10 [M+H]⁺. ¹H NMR (500 MHz, DMSO-d₆) δ 9.63 (s, 1H), 8.05-8.00 (m, 1H), 8.01-7.96 (m, 1H), 7.89-7.83 (m, 1H), 7.50-7.39 (m, 4H), 7.35-7.31 (m, 1H), 7.21 (d, J=3.8 Hz, 1H), 2.38 (s, 3H), 2.20 (s, 3H).

### Synthesis of N-[2-(5-cyanothiophen-2-yl)-3-methylphenyl]-N,3-dimethyl-1-benzothiophene-2-carboxamide

According to the general procedure for N-alkylation of amides (Method B). N-[2-(5-cyanothiophen-2-yl)-3-methylphenyl]-3-methyl-1-benzothiophene-2-carboxamide (18 mg, 0.05 mmol). Purification using preparative HPLC Method B. Solvent was concentrated under vacuo to give the desired product as a colorless oil (16.7 mg, 89% yield). LCMS (220 nm, 254 nm): t_{R} 3.167 min, purity ≥95%, m/z (ESI): 403.10 [M+H]⁺.

### Synthesis of N-3-dimethyl-N-{3-methyl-2-[5-(1H-1,2,3,4-tetrazol-5-yl)thiophen-2-yl]phenyl}-1-benzothiophene-2-carboxamide (RU-NT-446)

According to general procedure for tetrazole formation. N-[2-(5-cyanothiophen-2-yl)-3-methylphenyl]-N,3-dimethyl-1-benzothiophene-2-carboxamide (16 mg, 0.04 mmol). Temperature: 50° C. Purification using preparative HPLC Method B. Solvent was concentrated under vacuo to give the desired product as a white solid (3.3 mg, 19% yield). LCMS (220 nm, 254 nm): t_{R} 2.733 min, purity ≥95%, m/z (ESI): 446.15 [M+H]⁺. ¹H NMR for the observed rotamers (500 MHz, DMSO-d₆) δ 17.08 (br s, 1H), 8.06-7.66 (m, 3H), 7.61-7.11 (m, 4H), 7.00 (d, J=3.7 Hz, 1H), 6.53 (s, 1H), 3.15 (s, 3H), 2.34 (s, 3H), 2.17 (s, 3H).

### Synthesis of N-(2-bromo-3-methylphenyl)-5-cyanothiophene-2-carboxamide

According to general procedure B for the amide coupling. 5-Cyanothiophene-2-carboxylic acid (0.4 g, 2.61 mmol), 2-bromo-3-methylaniline (0.49 g, 2.61 mmol), EtOAc (5 mL). Extraction was carried out 3x using EtOAc and brine and combined organic layers were collected and dried over anhydrous MgSO₄. The organic layers were concentrated under vacuum to give the desired product as a beige solid (0.8 g, 95% yield). LCMS (220 nm, 254 nm): t_{R} 2.641 min, purity ≥95%, m/z (ESI): 322.95 [M+H]⁺.

### Synthesis of methyl 5-[2-(5-cyanothiophene-2-amido)-6-methylphenyl]thiophene-2-carboxylate

According to the general procedure for Suzuki coupling reactions using Method A, precatalyst C1. N-(2-bromo-3-methylphenyl)-5-cyanothiophene-2-carboxamide (0.2 g, 0.62 mmol), [5-(methoxycarbonyl)thiophen-2-yl]boronic acid (0.23 g, 1.25 mmol), dry dioxane (12 mL). Temperature: 100° C. Purification using preparative HPLC Method B. Solvent was concentrated under vacuo to give the desired product as a yellow oil (88 mg, 37% yield). LCMS (220 nm, 254 nm): t_{R} 2.764 min, purity ≥95%, m/z (ESI): 383.05 [M+H]⁺.

### Synthesis of methyl 5-[2-methyl-6-(N-methyl-5-cyanothiophene-2-amido)phenyl]thiophene-2-carboxylate

According to the general procedure for N-alkylation of amides (Method B). Methyl 5-[2-(5-cyanothiophene-2-amido)-6-methylphenyl]thiophene-2-carboxylate (80 mg, 0.21 mmol), DMF (1 mL). Purification using preparative HPLC Method B. Solvent was concentrated under vacuo to give the desired product as white crystals (51 mg, 62% yield). LCMS (220 nm, 254 nm): t_{R} 2.831 min, purity ≥95%, m/z (ESI): 397.00 [M+H]⁺.

### Synthesis of 5-[2-methyl-6-(N-methyl-5-carbamoylthiophene-2-amido)phenyl]thiophene-2-carboxylic acid (RU-NT-447)

According to the general procedure for saponification of methyl esters. Methyl 5-[2-methyl-6-(N-methyl-5-cyanothiophene-2-amido)phenyl]thiophene-2-carboxylate (8 mg, 0.02 mmol). 50° C. O/N. Purification using preparative HPLC Method B. Solvent was concentrated under vacuo to give the desired product as a white solid (2.8 mg, 35% yield). LCMS (220 nm, 254 nm): t_{R} 1.975 min, purity ≥95%, m/z (ESI): 401.05 [M+H]⁺. ¹H NMR (500 MHz, DMSO-d₆) δ 13.19 (br s, 1H), 8.01 (s, 2H), 7.68 (d, J=3.7 Hz, 1H), 7.63-7.45 (m, 3H), 7.40 (t, J=4.7 Hz, 1H), 6.84 (d, J=3.7 Hz, 1H), 6.64 (d, J=4.0 Hz, 1H), 3.07 (s, 3H), 2.18 (s, 3H).

### Synthesis of methyl 5-{2-methyl-6-[N-methyl-5-(]H-1,2,3,4-tetrazol-5-yl)thiophene-2-amido]phenyl}thiophene-2-carboxylate (R-NT-448)

A solution of methyl 5-[2-methyl-6-(N-methyl-5-cyanothiophene-2-amido)phenyl]thiophene-2-carboxylate (40 mg, 0.1 mmol) in DMF (1 mL) was stirred at 50° C. in a 20 mL scintillation vial. Sodium azide (22 mg, 0.25 mmol) and ammonium chloride (1.5eq, 8 mg) were added and stirring continued at 50° C. O/N. Purification using preparative HPLC Method B. Solvent was concentrated under vacuo to give the desired product as a white solid (20 mg, 45% yield). LCMS (220 nm, 254 nm): t_{R} 2.439 min, purity ≥95%, m/z (ESI): 440.10 [M+H]⁺. ¹H NMR (500 MHz, DMSO-d₆) δ 17.46 (br s, 1H), 7.78 (d, J=3.8 Hz, 1H), 7.70-7.37 (m, 4H), 6.91 (d, J=3.8 Hz, 1H), 6.66 (d, J=4.0 Hz, 1H), 3.79 (s, 3H), 3.10 (s, 3H), 2.19 (s, 3H).

### Synthesis of 5-{2-methyl-6-[N-methyl-5-(]H-1,2,3,4-tetrazol-5-yl)thiophene-2-amido]phenyl}thiophene-2-carboxylic acid (RU-NT-449)

According to the general procedure for saponification of methyl esters. Methyl 5-{2-methyl-6-[N-methyl-5-(1H-1,2,3,4-tetrazol-5-yl)thiophene-2-amido]phenyl}thiophene-2-carboxylate (10 mg, 0.02 mmol). Purification using preparative HPLC Method B. Solvent was concentrated under vacuo to give the desired product as a white solid (8.2 mg, 85% yield). LCMS (220 nm, 254 nm): t_{R} 2.127 min, purity ≥95%, m/z (ESI): 426.00 [M+H]⁺. ¹H NMR (500 MHz, DMSO-d₆) δ 13.19 (br s, 1H), 7.68 (d, J=3.7 Hz, 1H), 7.55 (t, J=4.4 Hz, 1H), 7.47 (d, J=4.5 Hz, 2H), 6.87 (d, J=3.7 Hz, 1H), 6.68 (d, J=4.0 Hz, 1H), 3.11 (s, 3H), 2.19 (s, 3H).

### Synthesis of 5-acetyl-N-(2-bromo-3-methylphenyl)thiophene-2-carboxamide

According to general procedure B for the amide coupling. 5-Acetylthiophene-2-carboxylic acid (0.2 g, 1.18 mmol), 2-bromo-3-methylaniline (0.26 g, 1.41 mmol), DCM (5 mL). Temperature: 60° C. Purification using preparative HPLC Method B. Solvent was concentrated under vacuo to give the desired product as a beige solid (56 mg, 14% yield). LCMS (220 nm, 254 nm): t_{R} 2.560 min, purity ≥95%, m/z (ESI): 339.95 [M+H]⁺.

### Synthesis of 5-[2-(5-acetylthiophene-2-amido)-6-methylphenyl]thiophene-2-carboxylic acid (RU-NT-428)

According to the general procedure for Suzuki coupling reactions using Method A, precatalyst C1. 5-Acetyl-N-(2-bromo-3-methylphenyl)thiophene-2-carboxamide (56 mg, 0.17 mmol), [5-(methoxycarbonyl)thiophen-2-yl]boronic acid (62 mg, 0.33 mmol). Temperature: 100° C. Purification using preparative HPLC Method B. Solvent was concentrated under vacuo to give the desired product as a white solid (6 mg, 9% yield). LCMS (220 nm, 254 nm): t_{R} 2.287 min, purity ≥95%, m/z (ESI): 386.25 [M+H]⁺. ¹H NMR (500 MHz, DMSO-d₆) δ 13.08 (br s, 1H), 9.97 (s, 1H), 7.89 (d, J=4.0 Hz, 1H), 7.68 (d, J=3.8 Hz, 2H), 7.42 (d, J=7.7 Hz, 1H), 7.39-7.22 (m, 2H), 7.05 (d, J=3.8 Hz, 1H), 2.54 (s, 3H), 2.22 (s, 3H).

### Synthesis of methyl 5-[2-methyl-6-(N-methyl-5-acetylthiophene-2-amido)phenyl]thiophene-2-carboxylate

According to the general procedure for N-alkylation of amides (Method B). 5-[2-(5-Acetylthiophene-2-amido)-6-methylphenyl]thiophene-2-carboxylic acid (30 mg, 0.08 mmol), DMF (1 mL). Purification using preparative HPLC Method B. Solvent was concentrated under vacuo to give the desired product as an off-white solid (30 mg, 93% yield). LCMS (220 nm, 254 nm): t_{R} 2.689 min, purity ≥95%, m/z (ESI): 414.10 [M+H]⁺.

### Synthesis of 5-[2-methyl-6-(N-methyl-5-acetylthiophene-2-amido)phenyl]thiophene-2-carboxylic acid (RU-NT-450)

According to the general procedure for saponification of methyl esters. Methyl 5-[2-methyl-6-(N-methyl-5-acetylthiophene-2-amido)phenyl]thiophene-2-carboxylate (30 mg, 0.07 mmol). Purification using preparative HPLC Method B. Solvent was concentrated under vacuo to give the desired product as a white solid (8.1 mg, 28% yield). LCMS (220 nm, 254 nm): t_{R} 2.313 min, purity ≥95%, m/z (ESI): 400.0 [M+H]⁺. ¹H NMR (500 MHz, DMSO-d₆) δ 13.20 (br s, 1H), 7.68 (s, 1H), 7.51 (d, J=4.7 Hz, 2H), 7.43 (d, J=4.7 Hz, 2H), 6.85 (d, J=3.8 Hz, 1H), 6.68 (d, J=4.1 Hz, 1H), 3.09 (s, 3H), 2.47 (s, 3H), 2.18 (s, 3H).

### Synthesis of N-(2-bromo-3-methylphenyl)-5-(trifluoromethyl)thiophene-2-carboxamide

According to general procedure D for the amide coupling. 5-(Trifluoromethyl)thiophene-2-carboxylic acid (0.17 g, 0.87 mmol), THF (2 mL), 2-bromo-3-methylaniline (0.16 g, 0.87 mmol). Purification using preparative HPLC Method B. Solvent was concentrated under vacuo to give the desired product as a white solid (31.1 mg, 10% yield). LCMS (220 nm, 254 nm): t_{R} 3.157 min, purity ≥95%, m/z (ESI): 412.20 [M+H]⁺. ¹H NMR (500 MHz, DMSO-d₆) δ 10.46 (s, 1H), 8.06 (s, 1H), 7.86 (d, J=3.9 Hz, 1H), 7.65-7.12 (m, 2H) (s, 2H), 6.52 (s, 1H), 2.42 (s, 3H).

### Synthesis of methyl 5-{2-methyl-6-[5-(trifluoromethyl)thiophene-2-amido]phenyl}thiophene-2-carboxylate

According to the general procedure for Suzuki coupling reactions using Method A, precatalyst C1. Methyl 5-{2-methyl-6-[5-(trifluoromethyl)thiophene-2-amido]phenyl}thiophene-2-carboxylate (40 mg, 0.09 mmol), DMF (1 mL). Purification using preparative HPLC Method B. Solvent was concentrated under vacuo to give the desired product as a colorless oil (30.9 mg, 75% yield). LCMS (220 nm, 254 nm): t_{R} 3.165 min, purity ≥95%, m/z (ESI): 426.35 [M+H]⁺.

### Synthesis of 5-{2-methyl-6-[5-(trifluoromethyl)thiophene-2-amido]phenyl}thiophene-2-carboxylic acid (RU-NT-413)

Methyl 5-{2-methyl-6-[5-(trifluoromethyl)thiophene-2-amido]phenyl}thiophene-2-carboxylate (5 mg, 0.01 mmol). Purification using preparative HPLC Method B. Solvent was concentrated under vacuo to give the desired product as a white solid (4 mg, 83% yield). LCMS (220 nm, 254 nm): t_{R} 2.716 min, purity ≥95%, m/z (ESI): 412.10 [M+H]⁺. ¹H NMR (500 MHz, DMSO-d₆) δ 13.09 (br s, 1H), 10.12 (s, 1H), 8.07-7.55 (m, 3H), 7.42 (t, J=7.8 Hz, 1H), 7.33 (dd, J=12.7, 7.7 Hz, 1H), 7.05 (d, J=3.8 Hz, 2H), 2.22 (s, 1H). ¹⁹F NMR (471 MHz, DMSO-d₆)-54.58 Hz.

### Synthesis of methyl 5-{2-methyl-6-[N-methyl-5-(trifluoromethyl)thiophene-2-amido]phenyl}thiophene-2-carboxylate

According to the general procedure for N-alkylation of amides (Method B). Methyl 5-{2-methyl-6-[5-(trifluoromethyl)thiophene-2-amido]phenyl}thiophene-2-carboxylate (40 mg, 0.09 mmol), DMF (1 mL). Purification using preparative HPLC Method B. Solvent was concentrated under vacuo to give the desired product as a colorless oil (30.9 mg, 75% yield). LCMS (220 nm, 254 nm): t_{R} 3.207 min, purity ≥95%, m/z (ESI): 440.10 [M+H]⁺.

### Synthesis of 5-{2-methyl-6-[N-methyl-5-(trifluoromethyl)thiophene-2-amido]phenyl}thiophene-2-carboxylic acid (RU-NT-451)

According to the general procedure for saponification of methyl esters. Methyl 5-{2-methyl-6-[N-methyl-5-(trifluoromethyl)thiophene-2-amido]phenyl}thiophene-2-carboxylate (30 mg, 0.07 mmol). Purification using preparative HPLC Method B. Solvent was concentrated under vacuo to give the desired product as a white solid (20 mg, 69% yield). LCMS (220 nm, 254 nm): t_{R} 2.787 min, purity ≥95%, m/z (ESI): 467.10 [M+H]⁺. ¹H NMR (500 MHz, DMSO-d₆) δ 13.20 (br s, 1H), 7.69 (d, J=3.7 Hz, 1H), 7.53 (t, J=5.9 Hz, 3H), 7.47 (t, J=4.7 Hz, 1H), 6.86 (d, J=3.8 Hz, 1H), 6.53-6.31 (m, 1H), 3.12 (s, 3H), 2.19 (s, 3H). ¹⁹F NMR (471 MHz, DMSO-d₆) δ-54.57 Hz.

### Synthesis of methyl 5-[(2-bromo-3-methylphenyl)carbamoyl]thiophene-2-carboxylate

According to general procedure B for the amide coupling. 5-(Methoxycarbonyl)thiophene-2-carboxylic acid (0.4 g, 2.15 mmol), 2-bromo-3-methylaniline (0.4 g, 1 eq), DCM (5 mL), 60° C. O/N. Purification using preparative HPLC Method B. Solvent was concentrated under vacuo to give the desired product as a white crystals (0.25 g, 33% yield). LCMS (220 nm, 254 nm): t_{R} 2.816 min, purity ≥95%, m/z (ESI): 354.15 [M+H]⁺. ¹H NMR (500 MHz, DMSO-d₆) δ 10.36 (s, 1H), 8.02 (d, J=3.9 Hz, 1H), 7.88 (d, J=3.9 Hz, 1H), 7.41-7.10 (m, 3H), 3.87 (s, 3H), 2.42 (s, 3H).

### Synthesis of 5-{2-[5-(methoxycarbonyl)thiophene-2-amido]-6-methylphenyl}thiophene-2-carboxylic acid & methyl 5-{2-[5-(methoxycarbonyl)thiophene-2-amido]-6-methylphenyl}thiophene-2-carboxylate (RU-NT-445)

According to the general procedure for Suzuki coupling reactions using Method A, precatalyst C1. Methyl 5-[(2-bromo-3-methylphenyl)carbamoyl]thiophene-2-carboxylate (0.1 g, 0.28 mmol), [5-(methoxycarbonyl)thiophen-2-yl]boronic acid (60 mg, 0.31 mmol). Temperature: 100° C. LCMS of the crude showed a mixture of the monoester, the diester and the diacid. Purification using preparative HPLC Method B. Solvent was concentrated under vacuo to give the monoester as a white solid (16.9 mg, 15% yield). LCMS of monoester (220 nm, 254 nm): t_{R} 2.496 min, purity ≥95%, m/z (ESI): 402.20 [M+H]⁺. LCMS of diester (220 nm, 254 nm): t_{R} 2.857 min, purity ≥95%, m/z (ESI): 416.30 [M+H]⁺.

### Synthesis of 5-[2-(5-carboxythiophene-2-amido)-6-methylphenyl]thiophene-2-carboxylic acid (RU-NT-402)

According to the general procedure for saponification of methyl esters. 5-{2-[5-(Methoxycarbonyl)thiophene-2-amido]-6-methylphenyl}thiophene-2-carboxylic acid (16.9 mg, 0.04 mmol). Temperature: 50° C., O/N. Purification using preparative HPLC Method B. Solvent was concentrated under vacuo to give the desired product as a white solid (8 mg, 49% yield). LCMS (220 nm, 254 nm): t_{R} 2.816 min, purity ≥95%, m/z (ESI): 388.25 [M+H]⁺. ¹H NMR (500 MHz, DMSO-d₆) δ 13.26 (br s, 2H), 9.95 (s, 1H), 7.68 (d, J=1.4 Hz, 2H), 7.63 (d, J=4.0 Hz, 1H), 7.40 (d, J=7.7 Hz, 1H), 7.33 (d, J=3.0 Hz, 2H), 7.05 (dd, J=3.8, 1.2 Hz, 1H), 2.21 (s, 3H).

### Synthesis of methyl 5-{2-methyl-6-[N-methyl-5-(methoxycarbonyl)thiophene-2-amido]phenyl}thiophene-2-carboxylate (RU-NT-452)

According to the general procedure for N-alkylation of amides (Method B). Methyl 5-{2-[5-(methoxycarbonyl)thiophene-2-amido]-6-methylphenyl}thiophene-2-carboxylate (25 mg, 0.06 mmol), DMF (1 mL). Purification using preparative HPLC Method B. Solvent was concentrated under vacuo to give the desired product as a white solid (12 mg, 46% yield). LCMS (220 nm, 254 nm): t_{R} 2.856 min, purity ≥95%, m/z (ESI): 493.10 [M+CH₃CN+Na, 100%]⁺. ¹H NMR (500 MHz, MeOD-d₄) δ 7.73 (d, J=3.8 Hz, 1H), 7.63-7.45 (m, 3H), 7.45-7.34 (m, 1H), 6.75 (d, J=4.1 Hz, 1H), 6.71 (d, J=3.8 Hz, 1H), 3.86 (s, 3H), 3.84 (s, 3H), 3.23 (s, 3H), 2.22 (s, 3H).

### Synthesis of 5-[2-methyl-6-(N-methyl-5-carboxythiophene-2-amido)phenyl]thiophene-2-carboxylic acid (RU-NT-403)

According to the general procedure for saponification of methyl esters. Crude product from the previous step, methyl 5-{2-methyl-6-[N-methyl-5-(methoxycarbonyl)thiophene-2-amido]phenyl}thiophene-2-carboxylate, Temperature: 50° C. for 2 hours. Purification using preparative HPLC Method B. Solvent was concentrated under vacuo to give the desired product as a white solid was collected (6 mg, 8% yield assuming quantitative yield from the previous step). LCMS (220 nm, 254 nm): t_{R} 2.147 min, purity ≥95%, m/z (ESI): 402.25 [M+H]⁺. ¹H NMR (500 MHz, DMSO-d₆) δ 13.36 (s, 2H), 7.68 (d, J=3.7 Hz, 1H), 7.52 (d, J=5.2 Hz, 2H), 7.47 (d, J=4.1 Hz, 1H), 7.43 (dd, J=5.3, 3.9 Hz, 1H), 6.84 (d, J=3.7 Hz, 1H), 6.62 (d, J=4.1 Hz, 1H), 3.09 (s, 3H), 2.18 (s, 3H)

### Synthesis of 2-iodo-3,5-dimethylaniline

A 10 mL round-bottom flask was charged with 2-iodo-1,5-dimethyl-3-nitrobenzene (1.0 g, 3.61 mmol), reduced iron powder (1.25 g, 22.38 mmol), conc, HCl (12 M, 2 eq), and ethanol/water (10:1/v/v). The mixture was stirred at 80° C. for 4 h. Filtration was carried out under pressure to remove the iron powder prior and solvent was reduced under vacuo. Extraction was carried out using saturated NaHCO₃, EtOAc and brine 3x. The combined organic extract was dried over anhydrous MgSO₄ and reduced under vacuo to give the desired product as a brown solid which was used directly in the next step without further purification (0.77 g, 86% yield). LCMS (220 nm, 254 nm): t_{R} 2.231 min, purity ≥95%, m/z (ESI): 248.00 [M+H]⁺. ¹H NMR (500 MHz, DMSO-d₆) δ 6.40 (s, 1H), 6.37 (s, 1H), 5.09 (s, 2H), 2.25 (s, 3H), 2.10 (s, 3H).

### Synthesis of methyl 5-[(2-iodo-3,5-dimethylphenyl)carbamoyl]thiophene-2-carboxylate

According to general procedure A for the amide coupling. Step 1: 5-(Methoxycarbonyl)thiophene-2-carboxylic acid (330 mg, 1.62 mmol), DMF (1 mL). Step 2: methyl 5-(carbonochloridoyl)thiophene-2-carboxylate, EtOAc (5 mL), 2-iodo-3,5-dimethylaniline (400 mg, quantitative yield assumed from previous step). Purification using preparative HPLC Method B. Solvent was concentrated under vacuo to give the desired product as a brownish solid (89.3 mg, 13% yield). LCMS (220 nm, 254 nm): t_{R} 2.953 min, purity ≥95%, m/z (ESI): 415.95 [M+H]⁺. ¹H NMR (500 MHz, DMSO-d₆) δ 10.32 (s, 1H), 8.00 (d, J=4.0 Hz, 1H), 7.88 (d, J=4.0 Hz, 1H), 7.13 (d, J=2.1 Hz, 1H), 7.06 (d, J=2.1 Hz, 1H), 3.86 (s, 3H), 2.42 (s, 3H), 2.26 (s, 3H).

### Synthesis of 5-({2-[5-(methoxycarbonyl)thiophen-2-yl]-3,5-dimethylphenyl}carbamoyl)thiophene-2-carboxylic acid & 5-{2-[5-(methoxycarbonyl)thiophene-2-amido]-4,6-dimethylphenyl}thiophene-2-carboxylic acid

According to the general procedure for Suzuki coupling reactions using Method A, precatalyst C1. Methyl 5-[(2-iodo-3,5-dimethylphenyl)carbamoyl]thiophene-2-carboxylate (80 mg, 0.19 mmol), [5-(methoxycarbonyl)thiophen-2-yl]boronic acid (53.75 mg, 0.29 mmol). Temperature: 100° C. Purification using preparative HPLC Method B. Solvent was concentrated under vacuo to give the desired product as an off-white solid (50 mg, 63% yield). LCMS (220 nm, 254 nm): t_{R} 2.947 min, purity ≥95%, m/z (EI): 415.90 [M]⁺.

### Synthesis of 5-[2-(5-carboxythiophene-2-amido)-4,6-dimethylphenyl]thiophene-2-carboxylic acid (RU-NT-457)

According to the general procedure for saponification of methyl esters. 5-({2-[5-(methoxycarbonyl)thiophen-2-yl]-3,5-dimethylphenyl}carbamoyl)thiophene-2-carboxylic acid (10 mg, 0.02 mmol). Purification using preparative HPLC Method B. Solvent was concentrated under vacuo to give the desired product as a white solid (5.09 mg, 53% yield). LCMS (220 nm, 254 nm): t_{R} 2.523 min, purity ≥95%, m/z (ESI): 442.95 [M+H]⁺. ¹H NMR (500 MHz, DMSO-d₆) δ 13.48 (s, 1H), 10.25 (s, 1H), 7.97 (d, J=3.9 Hz, 1H), 7.78 (d, J=3.9 Hz, 1H), 7.12 (s, 1H), 7.06 (d, J=2.0 Hz, 1H), 2.42 (s, 3H), 2.26 (s, 3H).

### Synthesis of 5-({2-[5-(methoxycarbonyl)thiophen-2-yl]-3,5-dimethylphenyl}(methyl)carbamoyl)thiophene-2-carboxylic acid

According to the general procedure for N-alkylation of amides (Method B). 5-[2-(5-Carboxythiophene-2-amido)-4,6-dimethylphenyl]thiophene-2-carboxylic acid (40 mg, 0.09 mmol), DMF (1 mL). The crude was taken directly to the next step without further purification. LCMS (220 nm, 254 nm): t_{R} 2.945 min, purity ≥95%, m/z (ESI): 429.90 [M+H]⁺.

### Synthesis of 5-[2,4-dimethyl-6-(N-methyl-5-carboxythiophene-2-amido)phenyl]thiophene-2-carboxylic acid (RU-NT-458)

According to the general procedure for saponification of methyl esters. Crude methyl 5-{2,4-dimethyl-6-[N-methyl-5-(methoxycarbonyl)thiophene-2-amido]phenyl}thiophene-2-carboxylate. Purification using preparative HPLC Method B. Solvent was concentrated under vacuo to give the desired product as a white solid (38.69 mg, 100% yield quantitative yield from previous step).

LCMS (220 nm, 254 nm): t_{R} 2.509 min, purity ≥95%, m/z (ESI): 456.25 [M+CH₃CN, 100%]⁺. ¹H NMR (500 MHz, DMSO-d₆) δ 7.43 (d, J=4.0 Hz, 2H), 7.25 (d, J=13.8 Hz, 2H), 6.68 (s, 1H), 6.67 (s, 1H), 3.21 (s, 3H), 2.41 (s, 3H), 2.26 (s, 3H).

### Synthesis of N-(2-bromo-3-methylphenyl)-3-fluorothiophene-2-carboxamide

According to general procedure C for the amide coupling. 3-Fluorothiophene-2-carboxylic acid (250 mg, 1.58 mmol), 2-bromo-3-methylaniline (323 mg, 0.23 mmol), EtOAc (5 mL). Purification using preparative HPLC Method B. Solvent was concentrated under vacuo to give the desired product as beige crystals (120 mg, 24% yield). LCMS (220 nm, 254 nm): t_{R} 3.080 min, purity ≥95%, m/z (EI): 313.90 [M, 100%]⁺.

### Synthesis of methyl 5-[2-(3-fluorothiophene-2-amido)-6-methylphenyl]thiophene-2-carboxylate & 5-[2-(3-fluorothiophene-2-amido)-6-methylphenyl]thiophene-2-carboxylic acid (RU-NT-459)

According to the general procedure for Suzuki coupling reactions using Method A, precatalyst C1. N-(2-bromo-3-methylphenyl)-3-fluorothiophene-2-carboxamide (54.26 mg, 0.26 mmol), [5-(methoxycarbonyl)thiophen-2-yl]boronic acid (80 mg, 0.25 mmol). Temperature: 100° C. Purification using preparative HPLC Method B. Solvent was concentrated under vacuo to give the desired product as a clear oil (80 mg, 84% yield). LCMS (220 nm, 254 nm): t_{R} 3.061 min, purity ≥95%, m/z (ESI): 376.00 [M+H, 100%]⁺ as well as the acidified product as a white solid after overnight lyophilization (10 mg, 10% yield). LCMS (220 nm, 254 nm): t_{R} 2.580 min, purity ≥95%, m/z (ESI): 362.00 [M+H]⁺. ¹H-NMR of acid: (500 MHz, DMSO-d₆) δ 13.20 (br s, 1H), 8.57 (d, J=7.4 Hz, 1H), 7.97-7.87 (m, 1H), 7.84 (t, J=4.9 Hz, 1H), 7.80 (d, J=3.8 Hz, 1H), 7.40 (t, J=7.9 Hz, 1H), 7.21 (d, J=7.4 Hz, 1H), 7.17 (d, J=3.8 Hz, 1H), 7.09 (d, J=5.6 Hz, 1H), 2.16 (s, 3H). ¹⁹F NMR (471 MHz, MeOD) δ-123.41.

### Synthesis of 5-[2-methyl-6-(N-methyl-3-fluorothiophene-2-amido)phenyl]thiophene-2-carboxylic acid (RU-NT-456)

According to the general procedure for N-alkylation of amides (Method B). Methyl 5-[2-(3-fluorothiophene-2-amido)-6-methylphenyl]thiophene-2-carboxylate (13.5 mg, 0.04 mmol), DMF (1 mL). Purification using preparative HPLC Method B. Solvent was concentrated under vacuo to give the desired product as an off-white solid (6.7 mg, 50% yield). LCMS (220 nm, 254 nm): t_{R} 2.367 min, purity ≥95%, m/z (ESI): 376.05 [M+H]⁺. ¹H NMR (500 MHz, DMSO-d₆) δ 13.13 (br s, 1H), 7.71 (d, J=3.7 Hz, 1H), 7.61 (t, J=4.7 Hz, 2H), 7.40 (d, J=4.5 Hz, 1H), 7.28 (t, J=4.7 Hz, 2H), 6.88 (dd, J=20.5, 4.6 Hz, 1H), 3.02 (s, 3H), 2.17 (s, 3H). ¹⁹F NMR (471 MHz, MeOD) δ-117.30 Hz.

### Synthesis of N-(2-bromo-3-methylphenyl)-3-methoxythiophene-2-carboxamide

According to general procedure C for the amide coupling. 3-Methoxythiophene-2-carboxylic acid (250 mg, 1.8 mmol), 2-bromo-3-methylaniline (0.23 mL, 1.1 eq). Purification using preparative HPLC Method B. Solvent was concentrated under vacuo to give the desired product as a pink solid (23.9 mg, 5% yield). LCMS (220 nm, 254 nm): t_{R} 3.007 min, purity ≥95%, m/z (EI): 329.95 [M]⁺.

### Synthesis of 5-[2-(3-methoxythiophene-2-amido)-6-methylphenyl]thiophene-2-carboxylic acid (RU-NT-460)

According to the general procedure for Suzuki coupling reactions using Method A, precatalyst C1. N-(2-bromo-3-methylphenyl)-3-methoxythiophene-2-carboxamide (20 mg, 0.06 mmol), [5-(methoxycarbonyl)thiophen-2-yl]boronic acid (17.1 mg, 0.09 mmol). Temperature: 100° C. Purification using preparative HPLC Method B. Solvent was concentrated under vacuo to give the desired product as a white solid (1.53 mg, 53% yield). LCMS (220 nm, 254 nm): t_{R} 2.584 min, purity ≥95%, m/z (ESI): 374 [M+H]⁺. ¹H NMR for the observed rotamers (500 MHz, DMSO-d₆) δ 13.14 (br s, 1H), 9.39 (s, 1H), 7.97 (ddd, J=14.9, 6.1, 3.2 Hz, 2H), 7.75 (d, J=3.7 Hz, 1H), 7.64 (d, J=8.0 Hz, 1H), 7.53-7.37 (m, 3H), 7.29 (d, J=7.6 Hz, 1H), 7.14 (d, J=3.7 Hz, 1H), 2.19 (s, 3H), 1.91-1.75 (m, 1H), 1.24 (s, 1H), 1.04-0.89 (m, 1H), 0.69-0.49 (m, 2H).

### Synthesis of 5-[2-methyl-6-(N-methyl-3-methoxythiophene-2-amido)phenyl]thiophene-2-carboxylic acid (RU-NT-461)

According to the general procedure for N-alkylation of amides (Method B). 5-[2-(3-methoxythiophene-2-amido)-6-methylphenyl]thiophene-2-carboxylic acid (20 mg, 0.21 mmol). Purification using preparative HPLC Method B. Solvent was concentrated under vacuo to give the desired product as a white solid (3.1 mg, 4% yield). LCMS (220 nm, 254 nm): t_{R} 2.329 min, purity ≥95%, m/z (ESI): 388.05 [M+H]⁺. ¹H NMR for the observed rotamers (500 MHz, Methanol-d₄) δ 6.77 (d, J=3.6 Hz, 1H), 6.59 (d, J=5.6 Hz, 1H), 6.51-6.27 (m, 2H), 6.23 (d, J=7.9 Hz, 1H), 6.15-5.84 (m, 2H), 2.71 (s, 3H), 2.30 (d, J=65.6 Hz, 3H), 1.44 (s, 3H).

### Synthesis of N-(2-bromo-3-methylphenyl)-2H,3H-thieno[3,4-b][ ],4]dioxine-5-carboxamide

According to general procedure C for the amide coupling. 2H,3H-Thieno[3,4-b][1,4]dioxine-5-carbonyl chloride (329 mg, 1.61 mmol), 2-bromo-3-methylaniline (329 mg, 1.77 mol), EtOAc (5 mL). Purification using preparative HPLC Method B. Solvent was concentrated under vacuo to give the desired product as an off-white solid (20.8 mg, 4% yield). LCMS (220 nm, 254 nm): t_{R} 2.944 min, purity ≥95%, m/z (ESI): 355.90 [M+H]⁺.

### Synthesis of methyl 5-(2-methyl-6-{2H,3H-thieno[3,4-b][ ],4]dioxine-5-amido}phenyl)thiophene-2-carboxylate

According to the general procedure for Suzuki coupling reactions using Method A, precatalyst C1. N-(2-bromo-3-methylphenyl)-2H,3H-thieno[3,4-b][1,4]dioxine-5-carboxamide (21 mg, 0.06 mmol), 5-(methoxycarbonyl)thiophen-2-yl]boronic acid (16.54 mg, 0.09 mmol). Temperature: 100° C. Purification using preparative HPLC Method B. Solvent was concentrated under vacuo to give the desired product as a beige semi-solid (10.7 mg, 43% yield). LCMS (220 nm, 254 nm): t_{R} 3.079 min, purity ≥95%, m/z (EI): 415.25 [M]⁺.

### Synthesis of methyl 5-(2-methyl-6-{N-methyl-2H,3H-thieno[3,4-b][ ],4]dioxine-5-amido}phenyl)thiophene-2-carboxylate

According to the general procedure for N-alkylation of amides (Method B). Methyl 5-(2-methyl-6-{2H,3H-thieno[3,4-b][1,4]dioxine-5-amido}phenyl)thiophene-2-carboxylate (10.9 mg, 0.03 mmol), DMF (1 mL). Purification using preparative HPLC Method B. Solvent was concentrated under vacuo to give the desired product as a colorless oil (9.07 mg, 81% yield). LCMS (220 nm, 254 nm): t_{R} 2.705 min, purity ≥95%, m/z (ESI): 430.30 [M+H]⁺.

### Synthesis of 5-(2-methyl-6-{N-methyl-2H,3H-thieno[3,4-b][ ],4]dioxine-5-amido}phenyl)thiophene-2-carboxylic acid (RU-NT-462)

According to the general procedure for saponification of methyl esters. Methyl 5-(2-methyl-6-{N-methyl-2H,3H-thieno[3,4-b][1,4]dioxine-5-amido}phenyl)thiophene-2-carboxylate (9.07 mg, 0.02 mmol). Purification using preparative HPLC Method B. Solvent was concentrated under vacuo to give the desired product as a white solid (5.45 mg, 62% yield). LCMS (220 nm, 254 nm): t_{R} 2.347 min, purity ≥95%, m/z (ESI): 416.25 [M+H]⁺. ¹H NMR (500 MHz, DMSO-d₆) δ 13.06 (br s, 1H), 7.72 (d, J=3.8 Hz, 1H), 7.33 (d, J=7.2 Hz, 2H), 7.07 (s, 1H), 7.00 (d, J=3.7 Hz, 1H), 6.72 (s, 1H), 3.99 (d, J=39.5 Hz, 4H), 2.91 (s, 3H), 2.19 (s, 3H).

### Synthesis of N-(2-bromo-3-methylphenyl)-3-ethoxythiophene-2-carboxamide

According to general procedure C for the amide coupling. 3-Ethoxythiophene-2-carboxylic acid (250 mg, 1.45 mmol) was added 2-bromo-3-methylaniline (324 mg, 1.74 mmol), EtOAc (3 mL). Purification using preparative HPLC Method B. Solvent was concentrated under vacuo to give the desired product as a white solid (30 mg, 6% yield). LCMS (220 nm, 254 nm): t_{R} 3.191 min, purity ≥95%, m/z (ESI): 341.10 [M+H]⁺.

### Synthesis of methyl 5-[2-(3-ethoxythiophene-2-amido)-6-methylphenyl]thiophene-2-carboxylate

According to the general procedure for Suzuki coupling reactions using Method A, precatalyst C1. N-(2-bromo-3-methylphenyl)-3-ethoxythiophene-2-carboxamide (30 mg, 0.09 mmol), [5-(methoxycarbonyl)thiophen-2-yl]boronic acid (24.60 mg, 0.13 mmol). Purification using preparative HPLC Method B. Solvent was concentrated under vacuo to give the desired product as a white solid (21.9 mg, 62% yield). LCMS (220 nm, 254 nm): t_{R} 3.140 min, purity ≥95%, m/z (EI): 401.20 [M]⁺.

### Synthesis of methyl 5-[2-methyl-6-(N-methyl-3-ethoxythiophene-2-amido)phenyl]thiophene-2-carboxylate

According to the general procedure for N-alkylation of amides (Method B). Methyl 5-[2-(3-ethoxythiophene-2-amido)-6-methylphenyl]thiophene-2-carboxylate (15.9 mg, 0.04 mmol), DMF (1 mL). Purification using preparative HPLC Method B. Solvent was concentrated under vacuo to give the desired product as a colorless oil (8 mg, 49% yield). LCMS (220 nm, 254 nm): t_{R} 2.872 min, purity ≥95%, m/z (ESI): 438.30 [M+Na]+.

### Synthesis of 5-[2-methyl-6-(N-methyl-3-ethoxythiophene-2-amido)phenyl]thiophene-2-carboxylic acid (RU-NT-464)

According to the general procedure for saponification of methyl esters. Methyl 5-[2-methyl-6-(N-methyl-3-ethoxythiophene-2-amido)phenyl]thiophene-2-carboxylate (8 mg, 0.02 mmol). Purification using preparative HPLC Method B. Solvent was concentrated under vacuo to give the desired product as a white solid (5.6 mg, 72% yield). LCMS (220 nm, 254 nm): t_{R} 2.457 min, purity ≥95%, m/z (ESI): 423.25 [M+H]⁺. ¹H NMR (500 MHz, DMSO-d₆) δ 13.12 (br s, 1H), 7.72 (d, J=3.7 Hz, 1H), 7.53 (s, 1H), 7.28 (d, J=21.2 Hz, 2H), 7.02 (d, J=3.7 Hz, 2H), 6.79 (s, 1H), 4.37-3.72 (m, 2H), 2.99 (s, 3H), 2.20 (s, 3H), 1.57-0.78 (m, 3H).

### Synthesis of 5-[2-(3-ethoxythiophene-2-amido)-6-methylphenyl]thiophene-2-carboxylic acid (RU-NT-465)

According to the general procedure for saponification of methyl esters. Methyl 5-[2-(3-ethoxythiophene-2-amido)-6-methylphenyl]thiophene-2-carboxylate (6 mg, 0.01 mmol). Purification using preparative HPLC Method B. Solvent was concentrated under vacuo to give the desired product as a white solid (1.31 mg, 23% yield). LCMS (220 nm, 254 nm): t_{R} 2.653 min, purity ≥95%, m/z (EI): 387.2 [M]⁺. ¹H NMR (500 MHz, DMSO-d₆) δ 13.28 (br s, 1H), 8.83 (s, 1H), 8.19 (d, J=8.2 Hz, 1H), 7.89 (d, J=3.5 Hz, 1H), 7.79 (d, J=5.5 Hz, 1H), 7.37 (t, J=7.9 Hz, 1H), 7.21 (d, J=3.5 Hz, 1H), 7.12 (d, J=7.5 Hz, 1H), 7.07 (d, J=5.5 Hz, 1H), 4.04 (q, J=7.0 Hz, 2H), 2.10 (s, 3H), 1.08 (t, J=7.0 Hz, 3H).

### Synthesis of N-(2-bromo-3-methylphenyl)-3-cyanothiophene-2-carboxamide

According to general procedure C for the amide coupling. 3-Cyanothiophene-2-carboxylic acid (0.25 g, 1.63 mmol) was added 2-bromo-3-methylaniline (330 mg, 1.80 mmol), EtOAc (5 mL). Purification using preparative HPLC Method B. Solvent was concentrated under vacuo to give the desired product as a white solid (140 mg, 27% yield). LCMS (220 nm, 254 nm): t_{R} 2.587 min, purity ≥95%, m/z (ESI): 322.95 [M+H]⁺.

### Synthesis of methyl 5-[2-(3-cyanothiophene-2-amido)-6-methylphenyl]thiophene-2-carboxylate

According to the general procedure for Suzuki coupling reactions using Method A, precatalyst C1. [5-(methoxycarbonyl)thiophen-2-yl]boronic acid (76.44 mg, 0.41 mmol), N-(2-bromo-3-methylphenyl)-3-cyanothiophene-2-carboxamide (120 mg, 0.37 mmol). Purification using preparative HPLC Method B. Solvent was concentrated under vacuo to give the desired product as a beige solid (60 mg, 42% yield). LCMS (220 nm, 254 nm): t_{R} 2.143 min, purity ≥95%, m/z (ESI): 383.05 [M+H]⁺.

### Synthesis of methyl 5-[2-methyl-6-(N-methyl-3-cyanothiophene-2-amido)phenyl]thiophene-2-carboxylate

According to the general procedure for N-alkylation of amides (Method B). Methyl 5-[2-(3-cyanothiophene-2-amido)-6-methylphenyl]thiophene-2-carboxylate (60 mg, 0.16 mmol), DMF (1 mL). Purification using preparative HPLC Method B. Solvent was concentrated under vacuo to give the desired product as a white solid (46.2 mg, 74% yield). LCMS (220 nm, 254 nm): t_{R} 2.676 min, purity ≥95%, m/z (ESI): 397.05 [M+H]⁺.

### Synthesis of 5-[2-methyl-6-(N-methyl-3-cyanothiophene-2-amido)phenyl]thiophene-2-carboxylic acid (RU-NT-454)

According to the general procedure for saponification of methyl esters. Methyl 5-[2-methyl-6-(N-methyl-3-cyanothiophene-2-amido)phenyl]thiophene-2-carboxylate (10 mg, 0.03 mmol). Purification using preparative HPLC Method B. Solvent was concentrated under vacuo to give the desired product as a white solid (6.2 mg, 61% yield). LCMS (220 nm, 254 nm): t_{R} 2.281 min, purity ≥95%, m/z (ESI): 383.00 [M+H]⁺. ¹H NMR (500 MHz, DMSO-d₆) δ 13.19 (br s, 1H), 7.80 (d, J=5.2 Hz, 1H), 7.70 (d, J=3.7 Hz, 1H), 7.50 (d, J=4.9 Hz, 2H), 7.45 (dd, J=11.6, 4.9 Hz, 2H), 6.83 (d, J=3.8 Hz, 1H), 3.14 (s, 3H), 2.16 (s, 3H).

### Synthesis of methyl 5-{2-methyl-6-[N-methyl-3-(]H-1,2,3,4-tetrazol-5-yl)thiophene-2-amido]phenyl}thiophene-2-carboxylate (RU-NT-466)

In a 20 mL scintillation vial with magnetic stirrer methyl 5-[2-methyl-6-(N-methyl-3-cyanothiophene-2-amido)phenyl]thiophene-2-carboxylate (22.4 mg, 0.06 mmol) was dissolved in THF (1 mL), TBAF (7.39 mg, 0.03 mmol) and excess TMSN₃ (0.1 mL) were added. The vial was sealed and heated at 80° C. O/N. The crude reaction mixture was extracted with EtOAc and the organic layer washed with brine followed dried over Na₂SO₄ and concentrated under reduced pressure. Purification using preparative HPLC Method B. Solvent was concentrated under vacuo to give the desired product as a white solid (12 mg, 53% yield). LCMS (220 nm, 254 nm): t_{R} 2.528 min, purity ≥95%, m/z (ESI): 440.30 [M+H]⁺. ¹H NMR for the observed rotamers (500 MHz, DMSO-d₆) δ 16.42 (br s, 1H), 7.99-7.67 (m, 2H), 7.64-7.49 (m, 1H), 7.42 (d, J=7.4 Hz, 1H), 7.34 (t, J=5.6 Hz, 1H), 7.24 (d, J=4.6 Hz, 1H), 7.15 (dd, J=11.5, 3.8 Hz, 1H), 3.83 (s, 3H), 3.00 (s, 3H), 2.19 (s, 3H).

### Synthesis of 5-{2-methyl-6-[N-methyl-3-(]H-1,2,3,4-tetrazol-5-yl)thiophene-2-amido]phenyl}thiophene-2-carboxylic acid (RU-NT-467)

According to the general procedure for saponification of methyl esters. Methyl 5-{2-methyl-6-[N-methyl-3-(1H-1,2,3,4-tetrazol-5-yl)thiophene-2-amido]phenyl}thiophene-2-carboxylate (8.8 mg, 0.02 mmol). Purification using preparative HPLC Method B. Solvent was concentrated under vacuo to give the desired product as a white solid (5.2 mg, 61% yield). LCMS (220 nm, 254 nm): t_{R} 2.167 min, purity ≥95%, m/z (EI): 425.25 [M]⁺. ¹H NMR for the observed rotamers (500 MHz, DMSO-d₆) δ 16.23 (br s, 1H), 7.78 (d, J=3.7 Hz, 11H), 7.74 (d, J=5.1 Hz, 1H), 7.61-7.54 (m, 1H), 7.41 (d, J=7.3 Hz, 1H), 7.33 (dd, J=9.5, 4.9 Hz, 2H), 7.22 (d, J=4.5 Hz, 1H), 7.10 (d, J=3.7 Hz, 1H), 3.00 (s, 3H), 2.19 (s, 3H).

### Synthesis of N-(2-bromo-3-methylphenyl)-5-fluoro-3-methyl-1-benzothiophene-2-carboxamide

According to general procedure C for the amide coupling. 5-Fluoro-3-methyl-1-benzothiophene-2-carboxylic acid (100 mg, 0.48 mmol), 2-bromo-3-methylaniline (106.2 mg, 0.57 mmol), EtOAc (5 mL). Purification using preparative HPLC Method B. Solvent was concentrated under vacuo to give the desired product as a white solid (35.7 mg, 20% yield). LCMS (220 nm, 254 nm): t_{R} 3.487 min, purity ≥95%, m/z (ESI): 379.15 [M+H]⁺.

### Synthesis of methyl 5-[2-(5-fluoro-3-methyl-1-benzothiophene-2-amido)-6-methylphenyl]thiophene-2-carboxylate

According to the general procedure for Suzuki coupling reactions using Method A, precatalyst C1. N-(2-bromo-3-methylphenyl)-5-fluoro-3-methyl-1-benzothiophene-2-carboxamide (35.7 mg, 0.09 mmol), [5-(methoxycarbonyl)thiophen-2-yl]boronic acid (26.3 mg, 0.14 mmol). Temperature: 100° C.). Purification using preparative HPLC Method B. Solvent was concentrated under vacuo to give the desired product as a clear oil (18.3 mg, 44% yield). LCMS (220 nm, 254 nm): t_{R} 3.369 min, purity ≥95%, m/z (ESI): 440.30 [M+H]⁺.

### Synthesis of methyl 5-[2-methyl-6-(N-methyl-5-chloro-3-methyl-1-benzothiophene-2-amido)phenyl]thiophene-2-carboxylate (RU-NT-468)

According to the general procedure for saponification of methyl esters. Methyl 5-[2-(5-fluoro-3-methyl-1-benzothiophene-2-amido)-6-methylphenyl]thiophene-2-carboxylate (5 mg, 0.01 mmol). Purification using preparative HPLC Method B. Solvent was concentrated under vacuo to give the desired product as a white solid. ¹H-NMR (4 mg, 83% yield). LCMS (220 nm, 254 nm): t_{R} 2.911 min, purity ≥95%, m/z (ESI): 426.30 [M+H]⁺. ¹H NMR (500 MHz, DMSO-d₆) δ 13.10 (br s, 1H), 9.53 (s, 1H), 8.03 (dd, J=8.9, 4.9 Hz, 1H), 7.74 (d, J=3.7 Hz, 1H), 7.70 (dd, J=10.0, 2.6 Hz, 1H), 7.46 (d, J=7.9 Hz, 1H), 7.42 (t, J=7.7 Hz, 1H), 7.35 (td, J=8.9, 2.6 Hz, 1H), 7.31 (d, J=7.4 Hz, 1H), 7.09 (d, J=3.7 Hz, 1H), 2.31 (s, 3H), 2.19 (s, 3H). ¹⁹F NMR (471 MHz, DMSO-d₆) δ-117.46 Hz.

### Synthesis of methyl 5-[2-methyl-6-(N-methyl-5-fluoro-3-methyl-]-benzothiophene-2-amido)phenyl]thiophene-2-carboxylate

According to the general procedure for N-alkylation of amides (Method B). Methyl 5-[2-(5-fluoro-3-methyl-1-benzothiophene-2-amido)-6-methylphenyl]thiophene-2-carboxylate (13.3 mg, 0.03 mmol), DMF (1 mL). Purification using preparative HPLC Method B. Solvent was concentrated under vacuo to give the desired product as a white solid (6.8 mg, 50% yield). LCMS (220 nm, 254 nm): t_{R} 3.256 min, purity ≥95%, m/z (ESI): 454.30 [M+H]⁺.

### Synthesis of 5-[2-methyl-6-(N-methyl-5-fluoro-3-methyl-]-benzothiophene-2-amido)phenyl]thiophene-2-carboxylic acid (RU-NT-469)

According to the general procedure for saponification of methyl esters. Methyl 5-[2-methyl-6-(N-methyl-5-fluoro-3-methyl-1-benzothiophene-2-amido)phenyl]thiophene-2-carboxylate (6.8 mg, 0.01 mmol). Purification using preparative HPLC Method B. Solvent was concentrated under vacuo to give the desired product as a white solid (6 mg, 91% yield). LCMS (220 nm, 254 nm): t_{R} 2.820 min, purity ≥95%, m/z (ESI): 480.35 [M+H]⁺. ¹H NMR for the observed rotamers (500 MHz, DMSO-d₆) δ 13.21 (br s, 1H), 8.07-7.81 (m, 1H), 7.82-7.49 (m, 2H), 7.49-7.05 (m, 4H), 6.87 (d, J=3.8 Hz, 1H), 3.13 (s, 3H), 2.31 (s, 3H), 2.13 (s, 3H). ¹⁹F NMR (471 MHz, DMSO-d₆) δ-117.89 Hz.

### Synthesis of 3-ethyl-I-benzothiophene-2-carboxylic acid

According to the general procedure for the synthesis of substituted benzothiophenes. Methyl 5-[2-(5-chloro-3-methyl-1-benzothiophene-2-amido)-6-methylphenyl]thiophene-2-carboxylate (10.9 mg, 0.02 mmol), DMF (1 mL). Purification using preparative HPLC Method B. Solvent was concentrated under vacuo to give the desired product as a colorless oil (7.3 mg, 65% yield). LCMS (220 nm, 254 nm): t_{R} 3.440 min, purity ≥95%, m/z (ESI): 471.30 [M+H]⁺.

### Synthesis of N-(2-bromo-3-methylphenyl)-3-ethyl-]-benzothiophene-2-carboxamide

According to general procedure A for the amide coupling. 3-Ethyl-1-benzothiophene-2-carboxylic acid (300 mg, 1.45 mmol) in step 1, thionyl chloride (2 mL). Step 2: 2-Bromo-6-methylaniline (375 mg, 2.02 mmol), DCM (3 mL), 3-ethyl-1-benzothiophene-2-carbonyl chloride (327 mg, quantitative yield assumed from previous step). Purification using preparative HPLC Method B. Solvent was concentrated under vacuo to give the desired product as a beige solid (60.5 mg, 9% yield). LCMS (220 nm, 254 nm): t_{R} 3.295 min, purity ≥95%, m/z (ESI): 471.30 [M+H]⁺. LCMS (220 nm, 254 nm, Rt=3.295 min), m/z (ESI): 375.00 [M+H, 100%]⁺.

### Synthesis of methyl 5-[2-(3-ethyl-]-benzothiophene-2-amido)-6-methylphenyl]thiophene-2-carboxylate

According to the general procedure for Suzuki coupling reactions using Method A, precatalyst C1. N-(2-bromo-3-methylphenyl)-3-ethyl-1-benzothiophene-2-carboxamide (26.9 mg, 0.07 mmol), [5-(methoxycarbonyl)thiophen-2-yl]boronic acid (41.4 mg, 3 eq), K₃PO₄(1.5 eq). Purification using preparative HPLC Method B. Solvent was concentrated under vacuo to give the desired product as a white solid (31.4 mg, 97% yield). LCMS (220 nm, 254 nm): t_{R} 3.501 min, purity ≥95%, m/z (ESI): 436.05 [M+H]⁺.

### Synthesis of 5-[2-(3-ethyl-]-benzothiophene-2-amido)-6-methylphenyl]thiophene-2-carboxylic acid (RU-NT-453)

According to the general procedure for saponification of methyl esters. Methyl 5-[2-(3-ethyl-1-benzothiophene-2-amido)-6-methylphenyl]thiophene-2-carboxylate (10 mg, 0.02 mmol). Purification using preparative HPLC Method B. Solvent was concentrated under vacuo to give the desired product as a white solid (4.2 mg, 43% yield). LCMS (220 nm, 254 nm): t_{R} 3.025 min, purity ≥95%, m/z (ESI): 422.05 [M+H]⁺. ¹H NMR (500 MHz, DMSO-d₆) δ 13.09 (br s, 1H), 9.48 (s, 1H), 8.03-7.94 (m, 1H), 7.92-7.82 (m, 1H), 7.75 (d, J=3.7 Hz, 1H), 7.51-7.35 (m, 4H), 7.33-7.23 (m, 1H), 7.09 (d, J=3.7 Hz, 1H), 2.91 (q, J=7.5 Hz, 2H), 2.19 (s, 3H), 1.07 (t, J=7.5 Hz, 3H).

### Synthesis of methyl 5-[2-methyl-6-(N-methyl-5-chloro-3-methyl-]-benzothiophene-2-amido)phenyl]thiophene-2-carboxylate

According to the general procedure for N-alkylation of amides (Method B). Methyl 5-[2-(5-chloro-3-methyl-1-benzothiophene-2-amido)-6-methylphenyl]thiophene-2-carboxylate (10.9 mg, 0.02 mmol), DMF (1 mL). Purification using preparative HPLC Method B. Solvent was concentrated under vacuo to give the desired product as a colorless oil (7.3 mg, 65% yield). LCMS (220 nm, 254 nm): t_{R} 3.440 min, purity ≥95%, m/z (ESI): 471.30 [M+H]⁺.

### Synthesis of 5-[2-methyl-6-(N-methyl-5-chloro-3-methyl-1-benzothiophene-2-amido)phenyl]thiophene-2-carboxylic acid (RU-NT-470)

According to the general procedure for saponification of methyl esters. Methyl 5-[2-methyl-6-(N-methyl-5-chloro-3-methyl-1-benzothiophene-2-amido)phenyl]thiophene-2-carboxylate (7.3 mg, 0.02 mmol). Purification using preparative HPLC Method B. Solvent was concentrated under vacuo to give the desired product as a white solid (8 mg, 86% yield). LCMS (220 nm, 254 nm): t_{R} 2.968 min, purity ≥95%, m/z (ESI): 457.25 [M+H, 100%]⁺. ¹H NMR for the observed rotamers (500 MHz, DMSO-d₆) δ 13.19 (br s, 1H), 7.93-7.82 (m, 2H), 7.79-7.67 (m, 1H), 7.39 (d, J=8.4 Hz, 2H), 7.36-7.26 (m, 2H), 6.85 (d, J=3.8 Hz, 1H), 3.13 (s, 3H), 2.30 (s, 3H), 2.07 (s, 3H).

### Synthesis of N-(2-bromo-3-methylphenyl)-5-chloro-3-methyl-1-benzothiophene-2-carboxamide

According to general procedure C for the amide coupling using T3P. 5-Chloro-3-methyl-1-benzothiophene-2-carboxylic acid (200 mg, 0.88 mmol) was added 2-bromo-3-methylaniline (1.2 eq), EtOAc (3 mL). Purification using preparative HPLC Method B. Solvent was concentrated under vacuo to give the desired product as a white solid (30.1 mg, 9% yield). LCMS (220 nm, 254 nm): t_{R} 3.700 min, purity ≥95%, m/z (ESI): 395.10 [M+H]⁺.

### Synthesis of methyl 5-[2-(5-chloro-3-methyl-1-benzothiophene-2-amido)-6-methylphenyl]thiophene-2-carboxylate

According to the general procedure for Suzuki coupling reactions using Method A, precatalyst C2. N-(2-bromo-3-methylphenyl)-5-chloro-3-methyl-1-benzothiophene-2-carboxamide (30 mg, 0.0732 mmol), [5-(methoxycarbonyl)thiophen-2-yl]boronic acid (14.14 mg, 0.08 mmol). Temperature: 100° C. Purification using preparative HPLC Method B. Solvent was concentrated under vacuo to give the desired product as a white solid (10.9 mg, 32% yield). LCMS (220 nm, 254 nm): t_{R} 3.551 min, purity ≥95%, m/z (ESI): 456.35 [M+H]⁺.

### Synthesis of methyl 5-[2-methyl-6-(N-methyl-5-chloro-3-methyl-1-benzothiophene-2-amido)phenyl]thiophene-2-carboxylate

According to the general procedure for N-alkylation of amides (Method B). Methyl 5-[2-(5-chloro-3-methyl-1-benzothiophene-2-amido)-6-methylphenyl]thiophene-2-carboxylate (10.9 mg, 0.02 mmol), DMF (1 mL). Purification using preparative HPLC Method B. Solvent was concentrated under vacuo to give the desired product as a colorless oil (7.3 mg, 65% yield). LCMS (220 nm, 254 nm): t_{R} 3.440 min, purity ≥95%, m/z (ESI): 471.30 [M+H]⁺.

### Synthesis of 5-[2-methyl-6-(N-methyl-5-chloro-3-methyl-]-benzothiophene-2-amido)phenyl]thiophene-2-carboxylic acid (RU-NT-471)

According to the general procedure for saponification of methyl esters. Methyl 5-[2-methyl-6-(N-methyl-5-chloro-3-methyl-1-benzothiophene-2-amido)phenyl]thiophene-2-carboxylate (7.3 mg, 0.02 mmol). Purification using preparative HPLC Method B. Solvent was concentrated under vacuo to give the desired product as a white solid (8 mg, 86% yield). LCMS (220 nm, 254 nm): t_{R} 2.968 min, purity ≥95%, m/z (ESI): 472.25 [M+H]⁺. ¹H NMR for the observed rotamers (500 MHz, DMSO-d₆) δ 13.19 (br s, 1H), 7.93-7.82 (m, 2H), 7.79-7.67 (m, 1H), 7.39 (d, J=8.4 Hz, 2H), 7.36-7.26 (m, 2H), 6.85 (d, J=3.8 Hz, 1H), 3.13 (s, 3H), 2.30 (s, 3H), 2.07 (s, 3H).

### Synthesis of N-(2-bromo-3-methylphenyl)-3-cyclopropyl-1-benzothiophene-2-carboxamide

According to general procedure A for the amide coupling using thionyl chloride. 3-Cyclopropyl-1-benzothiophene-2-carbonyl chloride (325 mg, 1.61 mmol), (3 mL), 3-cyclopropyl-1-benzothiophene-2-carbonyl chloride (325 mg, quantitative yield assumed from previous step). Purification using preparative HPLC Method B. Solvent was concentrated under vacuo to give the desired product as a beige semisolid (110 mg, 18% yield). LCMS (220 nm, 254 nm): t_{R} 3.635 min, purity ≥95%, m/z (ESI): 387 [M+H]⁺. ¹H NMR (500 MHz, DMSO-d₆) δ 9.83 (br s, 1H), 8.23-7.95 (m, 2H), 7.70 (d, J=8.0 Hz, 1H), 7.55-7.43 (m, 1H), 7.35 (t, J=7.8 Hz, 2H), 7.27 (d, J=7.5 Hz, 1H), 2.43 (s, 3H), 1.78-1.00 (m, 3H), 0.76 (d, J=5.2 Hz, 2H).

### Synthesis of methyl 5-[2-(3-cyclopropyl-]-benzothiophene-2-amido)-6-methylphenyl]thiophene-2-carboxylate

According to the general procedure for Suzuki coupling reactions using Method A, precatalyst C1. N-(2-bromo-3-methylphenyl)-3-cyclopropyl-1-benzothiophene-2-carboxamide (54.26 mg, 0.26 mmol), [5-(methoxycarbonyl)thiophen-2-yl]boronic acid (241.79 mg, 1.30 mmol), K₃PO₄ (1.5 eq). Purification using preparative HPLC Method B. Solvent was concentrated under vacuo to give the desired product as a white semi-solid (41 mg, 35% yield). LCMS (220 nm, 254 nm): t_{R} 3.468 min, purity ≥95%, m/z (ESI): 448.10 [M+H]⁺.

### Synthesis of 5-[2-(3-cyclopropyl-1-benzothiophene-2-amido)-6-methylphenyl]thiophene-2-carboxylic acid (RU-NT-460)

According to the general procedure for saponification of methyl esters. Methyl 5-[2-(3-cyclopropyl-1-benzothiophene-2-amido)-6-methylphenyl]thiophene-2-carboxylate (3 mg, 0.01 mmol). Purification using preparative HPLC Method B. Solvent was concentrated under vacuo to give the desired product as a white solid (1.53 mg, 53% yield). LCMS (220 nm, 254 nm): t_{R} 2.995 min, purity ≥95%, m/z (ESI): 434.0 [M+H]⁺. ¹H NMR (500 MHz, DMSO-d₆) δ 13.14 (br s, 1H), 9.39 (s, 1H), 7.97 (ddd, J=14.9, 6.1, 3.2 Hz, 2H), 7.75 (d, J=3.7 Hz, 1H), 7.64 (d, J=8.0 Hz, 1H), 7.53-7.37 (m, 3H), 7.29 (d, J=7.6 Hz, 1H), 7.14 (d, J=3.7 Hz, 1H), 2.19 (s, 3H), 1.91-1.75 (m, 1H), 1.24 (s, 1H), 1.04-0.89 (m, 1H), 0.69-0.49 (m, 2H).

### Synthesis of methyl 5-[2-methyl-6-(N-methyl-3-cyclopropyl-]-benzothiophene-2-amido)phenyl]thiophene-2-carboxylate

According to the general procedure for N-alkylation of amides (Method B). Methyl 5-[2-(3-cyclopropyl-1-benzothiophene-2-amido)-6-methylphenyl]thiophene-2-carboxylate (27.5 mg, 0.06 mmol), DMF (1 mL). Purification using preparative HPLC Method B. Solvent was concentrated under vacuo to give the desired product as a colorless oil (21.3 mg, 75% yield). LCMS (220 nm, 254 nm): t_{R} 3.320 min, purity ≥95%, m/z (ESI): 462.05 [M+H]⁺.

### Synthesis of 5-[2-methyl-6-(N-methyl-3-cyclopropyl-1-benzothiophene-2-amido)phenyl]thiophene-2-carboxylic acid (RU-NT-472)

According to the general procedure for saponification of methyl esters. Methyl 5-[2-methyl-6-(N-methyl-3-cyclopropyl-1-benzothiophene-2-amido)phenyl]thiophene-2-carboxylate (21.3 mg, 0.05 mmol). Purification using preparative HPLC Method B. Solvent was concentrated under vacuo to give the desired product as a white solid (13 mg, 63% yield). LCMS (220 nm, 254 nm): t_{R} 2.876 min, purity ≥95%, m/z (ESI): 448.35 [M+H]⁺. ¹H NMR (500 MHz, DMSO-d₆) indicated the presence of rotamers using VT-NMR experiment: δ 13.21 (br s, 1H), 7.99-7.86 (m, 1H), 7.79 (td, J=13.6, 5.7 Hz, 1H), 7.54 (t, J=7.8 Hz, 1H), 7.42 (tt, J=15.8, 8.2 Hz, 2H), 7.36-7.31 (m, 1H), 7.28 (dd, J=8.6, 5.3 Hz, 1H), 7.12 (d, J=3.7 Hz, 1H), 3.04 (s, 3H), 2.21 (s, 3H), 1.92 (td, J=13.2, 6.7 Hz, 1H), 1.00 (dd, J=17.8, 9.3 Hz, 2H), 0.84 (s, 1H), 0.65 (d, J=5.8 Hz, 1H).

### Synthesis of N-(2-bromo-3-methylphenyl)-3-ethylthiophene-2-carboxamide

According to general procedure C for the amide coupling using T3P. 3-Ethylthiophene-2-carboxylic acid (100 mg, 1.45 mmol) was added 2-bromo-3-methylaniline (324 mg, 1.74 mmol), EtOAc (3 mL). Purification using preparative HPLC Method B. Solvent was concentrated under vacuo to give the desired product as a beige semi-solid (45.9 mg, 10% yield). LCMS (220 nm, 254 nm): t_{R} 3.240 min, purity ≥95%, m/z (ESI): 326.00 [M+H]⁺.

### Synthesis of methyl 5-[2-(3-ethylthiophene-2-amido)-6-methylphenyl]thiophene-2-carboxylate

According to the general procedure for Suzuki coupling reactions using Method A, precatalyst C1. N-(2-bromo-3-methylphenyl)-3-ethylthiophene-2-carboxamide (45.9 mg, 0.14 mmol), [5-(methoxycarbonyl)thiophen-2-yl]boronic acid (39.49 mg, 0.21 mmol). Purification using preparative HPLC Method B. Solvent was concentrated under vacuo to give the desired product as a clear oil (28.9 mg, 53% yield). LCMS (220 nm, 254 nm): t_{R} 3.163 min, purity ≥95%, m/z (EI): 385.20 [M]⁺.

### Synthesis of 5-[2-(3-ethylthiophene-2-amido)-6-methylphenyl]thiophene-2-carboxylic acid (RU-NT-473)

According to the general procedure for saponification of methyl esters. Methyl 5-[2-(3-ethylthiophene-2-amido)-6-methylphenyl]thiophene-2-carboxylate (8 mg, 0.02 mmol). Purification using preparative HPLC Method B. Solvent was concentrated under vacuo to give the desired product as a white solid (5 mg, 65% yield). LCMS (220 nm, 254 nm): t_{R} 2.676 min, purity ≥95%, m/z (EI): 372.20 [M+H]⁺. ¹H NMR (500 MHz, DMSO-d₆) δ 13.09 (br s, 1H), 9.02 (s, 1H), 7.74 (d, J=3.8 Hz, 1H), 7.58 (d, J=5.0 Hz, 1H), 7.54-7.47 (m, 1H), 7.39 (t, J=7.8 Hz, 1H), 7.26 (dd, J=7.5, 1.2 Hz, 1H), 7.08 (d, J=3.7 Hz, 1H), 7.00 (d, J=5.1 Hz, 1H), 2.59 (q, J=7.5 Hz, 2H), 2.17 (s, 3H), 1.03 (t, J=7.5 Hz, 3H).

### Synthesis of methyl 5-[2-methyl-6-(N-methyl-3-ethylthiophene-2-amido)phenyl]thiophene-2-carboxylate

According to the general procedure for N-alkylation of amides (Method B). 5-[2-(3-Ethylthiophene-2-amido)-6-methylphenyl]thiophene-2-carboxylic acid (20.9 mg, 0.06 mmol), DMF (1 mL). Purification using preparative HPLC Method B. Solvent was concentrated under vacuo to give the desired product as a colorless oil (20.5 mg, 91% yield). LCMS (220 nm, 254 nm): t_{R} 3.063 min, purity ≥95%, m/z (EI): 399.20 [M]⁺.

### Synthesis of 5-[2-methyl-6-(N-methyl-3-ethylthiophene-2-amido)phenyl]thiophene-2-carboxylic acid (RU-NT-463)

According to the general procedure for saponification of methyl esters.5-[2-Methyl-6-(N-methyl-3-ethylthiophene-2-amido)phenyl]thiophene-2-carboxylate (15 mg, 0.04 mmol). Purification using preparative HPLC Method B. Solvent was concentrated under vacuo to give the desired product as a white solid (4 mg, 28% yield). LCMS (220 nm, 254 nm): t_{R} 2.613 min, purity ≥95%, m/z (ESI): 382.25 [M+H]⁺. ¹H NMR (500 MHz, DMSO-d₆) δ 13.17 (br s, 1H), 7.70 (s, 1H), 7.39 (d, J=27.5 Hz, 3H), 7.26 (d, J=7.5 Hz, 1H), 6.85 (d, J=36.1 Hz, 2H), 3.07 (s, 3H), 2.62 (q, J=22.2 Hz, 2H), 2.14 (s, 3H), 1.07 (t, J=8.0 Hz, 3H).

### Synthesis of N-(2-bromo-3-methylphenyl)-3-(trifluoromethyl)thiophene-2-carboxamide

According to general procedure C for the amide coupling using T3P. 3-(Trifluoromethyl)thiophene-2-carboxylic acid (250 mg, 1 eq), 2-bromo-3-methylaniline (260 mg, 1.4 mmol), EtOAc (5 mL). Purification using preparative HPLC Method B. Solvent was concentrated under vacuo to give the desired product as a white solid (190 mg, 41% yield). LCMS (220 nm, 254 nm): t_{R} 2.971 min, purity ≥95%, m/z (ESI): 365.90 [M+H]⁺.

### Synthesis of methyl 5-{2-methyl-6-[3-(trifluoromethyl)thiophene-2-amido]phenyl}thiophene-2-carboxylate

According to the general procedure for Suzuki coupling reactions using Method A, precatalyst C1. N-(2-bromo-3-methylphenyl)-3-(trifluoromethyl)thiophene-2-carboxamide (150 mg, 0.41 mmol), [5-(methoxycarbonyl)thiophen-2-yl]boronic acid (84.27 mg, 0.45 mmol). Purification using preparative HPLC Method B. Solvent was concentrated under vacuo to give the desired product as a yellow oil (100 mg, 57% yield). LCMS (220 nm, 254 nm): t_{R} 2.972 min, purity ≥95%, m/z (ESI): 426.05 [M+H]⁺.

### Synthesis of 5-{2-methyl-6-[3-(trifluoromethyl)thiophene-2-amido]phenyl}thiophene-2-carboxylic acid (RU-NT-455)

According to the general procedure for saponification of methyl esters. Methyl 5-{2-methyl-6-[3-(trifluoromethyl)thiophene-2-amido]phenyl}thiophene-2-carboxylate (30 mg, 0.07 mmol). Purification using preparative HPLC Method B. Solvent was concentrated under vacuo to give the desired product as a white solid (8.6 mg, 30% yield). LCMS (220 nm, 254 nm): t_{R} 2.560 min, purity ≥95%, m/z (ESI): 412.00 [M+H]⁺. ¹H NMR (500 MHz, DMSO-d₆) δ 13.08 (br s, 1H), 10.00 (s, 1H), 7.82 (d, J=5.2 Hz, 1H), 7.73 (d, J=3.8 Hz, 1H), 7.41 (t, J=7.7 Hz, 1H), 7.37 (d, J=5.2 Hz, 1H), 7.30 (dd, J=13.3, 7.7 Hz, 2H), 7.03 (d, J=3.8 Hz, 1H), 2.20 (s, 3H). ¹⁹F NMR (471 MHz, DMSO-d₆) δ-55.97 Hz.

### Synthesis of methyl 5-{2-methyl-6-[N-methyl-3-(trifluoromethyl)thiophene-2-amido]phenyl}thiophene-2-carboxylate

According to the general procedure for N-alkylation of amides (Method B). Methyl 5-{2-methyl-6-[3-(trifluoromethyl)thiophene-2-amido]phenyl}thiophene-2-carboxylate (8 mg, 0.02 mmol), DMF (1 mL). Purification using preparative HPLC Method B. Solvent was concentrated under vacuo to give the desired product as a colorless oil (8 mg, 97% yield). LCMS (220 nm, 254 nm): t_{R} 3.033 min, purity ≥95%, m/z (ESI): 440.00 [M+H]⁺.

### Synthesis of 5-{2-methyl-6-[N-methyl-3-(trifluoromethyl)thiophene-2-amido]phenyl}thiophene-2-carboxylic acid (RU-NT-474)

According to the general procedure for saponification of methyl esters. Methyl 5-{2-methyl-6-[N-methyl-3-(trifluoromethyl)thiophene-2-amido]phenyl}thiophene-2-carboxylate (25.5 mg, 0.06 mmol). Purification using preparative HPLC Method B. Solvent was concentrated under vacuo to give the desired product as a white solid (3.6 mg, 16% yield). LCMS (220 nm, 254 nm): t_{R} 2.603 min, purity ≥95%, m/z (ESI): 426.00 [M+H]⁺. ¹H NMR for the observed rotamers (500 MHz, DMSO-d₆) δ 13.18 (br s, 1H), 7.91-7.78 (m, 1H), 7.72 (dd, J=17.2, 4.5 Hz, 1H), 7.61-7.40 (m, 1H), 7.40-7.23 (m, 1H), 7.24-7.10 (m, 1H), 7.01 (d, J=3.8 Hz, 1H), 2.98 (d, J=68.0 Hz, 3H), 2.18 (d, J=16.6 Hz, 3H). ¹⁹F NMR (471 MHz, MeOD-d₄) δ-59.20 (d, J=520.0 Hz).

### Synthesis of N-(2-bromo-3-methylphenyl)-3-(difluoromethyl)thiophene-2-carboxamide

According to general procedure C for the amide coupling using T3P. 3-(Difluoromethyl)thiophene-2-carboxylic acid (250 mg, 1.4 mmol), 2-bromo-3-methylaniline (287.20 mg, 1.54 mmol), EtOAc (3 mL). Purification using preparative HPLC Method B. Solvent was concentrated under vacuo to give the desired product as a beige solid (440 mg, 91% yield). LCMS (220 nm, 254 nm): t_{R} 2.959 min, purity ≥95%, m/z (ESI): 345.00 [M−H]⁻.

### Synthesis of methyl 5-{2-[3-(difluoromethyl)thiophene-2-amido]-6-methylphenyl}thiophene-2-carboxylate

According to the general procedure for Suzuki coupling reactions using Method A, precatalyst C1. (2-bromo-3-methylphenyl)-3-(difluoromethyl)thiophene-2-carboxamide (200 mg, 0.58 mmol), [5-(methoxycarbonyl)thiophen-2-yl]boronic acid (128.94 mg, 0.69 mmol). Purification using preparative HPLC Method B. Solvent was concentrated under vacuo to give the desired product as a clear oil (100 mg, 43% yield). LCMS (220 nm, 254 nm): t_{R} 2.977 min, purity ≥95%, m/z (ESI): 429.25 [M+Na]⁺.

### Synthesis of 5-{2-[3-(difluoromethyl)thiophene-2-amido]-6-methylphenyl}thiophene-2-carboxylic acid (RU-NT-475)

According to the general procedure for saponification of methyl esters. Methyl 5-{2-[3-(difluoromethyl)thiophene-2-amido]-6-methylphenyl}thiophene-2-carboxylate (10 mg, 0.02 mmol). Purification using preparative HPLC Method B. Solvent was concentrated under vacuo to give the desired product as a white solid (6 mg, 62% yield). LCMS (220 nm, 254 nm): t_{R} 2.553 min, purity ≥95%, m/z (ESI): 394.20 [M+H]⁺. ¹H NMR (500 MHz, DMSO-d₆) δ 13.05 (s, 1H), 9.81 (s, 1H), 7.81 (d, J=5.1 Hz, 1H), 7.71 (d, J=3.7 Hz, 3H), 7.54-7.23 (m, 1H), 7.15 (s, 1H), 2.20 (s, 3H). ¹⁹F NMR (471 MHz, MeOD) δ-110.71 Hz.

### Synthesis of methyl 5-{2-methyl-6-[N-methyl-3-(difluoromethyl)thiophene-2-amido]phenyl}thiophene-2-carboxylate

According to the general procedure for N-alkylation of amides (Method B). Methyl 5-{2-[3-(difluoromethyl)thiophene-2-amido]-6-methylphenyl}thiophene-2-carboxylate (90 mg, 0.22 mmol), DMF (1 mL). Purification using preparative HPLC Method B. Solvent was concentrated under vacuo to give the desired product as a colorless oil (66.8 mg, 72% yield). LCMS (220 nm, 254 nm): t_{R} 3.005 min, purity ≥95%, m/z (ESI): 420.45 [M−H]⁻

### Synthesis of 5-{2-methyl-6-[N-methyl-3-(difluoromethyl)thiophene-2-amido]phenyl}thiophene-2-carboxylic acid (RU-NT-476)

According to the general procedure for saponification of methyl esters. Methyl 5-{2-methyl-6-[N-methyl-3-(difluoromethyl)thiophene-2-amido]phenyl}thiophene-2-carboxylate (65 mg, 0.15 mmol). Purification using preparative HPLC Method B. Solvent was concentrated under vacuo to give the desired product as a white solid was (17.4 mg, 28% yield). LCMS (220 nm, 254 nm): t_{R} 2.577 min, purity ≥95%, m/z (ESI): 408.25 [M+H]⁺. ¹H NMR (500 MHz, DMSO-d₆) δ 13.15 (br s, 1H), 7.68 (dd, J=8.1, 4.5 Hz, 2H), 7.45 (d, J=5.5 Hz, 2H), 7.37 (dd, J=5.3, 3.9 Hz, 1H), 7.22 (d, J=5.2 Hz, 1H), 7.05 (t, J=55.4 Hz, 1H), 6.84 (d, J=3.8 Hz, 1H), 2.17 (s, 3H). ¹⁹F NMR (471 MHz, MeOD-d₄) δ-109.02 (d, J=55.3 Hz), -109.67 (d, J=55.4 Hz), -111.99 (d, J=55.9 Hz), -112.64 (d, J=55.6 Hz).

### Synthesis of N-(2-bromo-3-methylphenyl)thieno[2,3-b]pyridine-2-carboxamide

According to general procedure C for the amide coupling using T3P. Thieno[2,3-b]pyridine-2-carboxylic acid (300 mg, 1.67 mmol), 2-bromo-3-methylaniline (373.79 mg, 2.01 mmol), EtOAc (3 mL) and DCM (2 mL). Extraction was carried out 3x using EtOAc and brine and combined organic layers were collected and dried over anhydrous MgSO₄. The organic layers were concentrated under vacuum to give the desired product as a beige solid (570 mg, 98% yield). LCMS (220 nm, 254 nm): t_{R} 2.603 min, purity ≥95%, m/z (ESI): 389.15 [M+CH₃CN]⁺.

### Synthesis of methyl 5-(2-methyl-6-{thieno[2,3-b]pyridine-2-amido}phenyl)thiophene-2-carboxylate

According to the general procedure for Suzuki coupling reactions using Method A, precatalyst C1. N-(2-bromo-3-methylphenyl)thieno[2,3-b]pyridine-2-carboxamide (300 mg, 0.86 mmol), [5-(methoxycarbonyl)thiophen-2-yl]boronic acid (192 mg, 1.04 mmol). Purification using preparative HPLC Method B. Solvent was concentrated under vacuo to give the desired product as a pink solid (179 mg, 51% yield). LCMS (220 nm, 254 nm): t_{R} 2.667 min, purity ≥95%, m/z (ESI): 449.35 [M+CH₃CN]⁺.

### Synthesis of 5-(2-methyl-6-{thieno[2,3-b]pyridine-2-amido}phenyl)thiophene-2-carboxylic acid (RU-NT-477)

According to the general procedure for saponification of methyl esters. Methyl 5-(2-methyl-6-{thieno[2,3-b]pyridine-2-amido}phenyl)thiophene-2-carboxylate (17 mg, 0.04 mmol). Purification using preparative HPLC Method B. Solvent was concentrated under vacuo to give the desired product as a white solid (16.2 mg, 99% yield). LCMS (220 nm, 254 nm): t_{R} 2.263 min, purity ≥95%, m/z (ESI): 395.20 [M+H]⁺. ¹H NMR (500 MHz, DMSO-d₆) δ 10.02 (s, 1H), 8.65 (dd, J=4.7, 1.6 Hz, 1H), 8.36 (dd, J=8.1, 1.7 Hz, 1H), 7.97 (s, 1H), 7.61-7.42 (m, 1H), 7.42-7.27 (m, 3H), 7.00 (d, J=3.7 Hz, 1H), 2.23 (s, 3H).

### Synthesis of methyl 5-(2-methyl-6-{N-methylthieno[2,3-b]pyridine-2-amido}phenyl)thiophene-2-carboxylate & 2-({2-[5-(methoxycarbonyl)thiophen-2-yl]-3-methylphenyl}(methyl)carbamoyl)-7-methylthieno[2,3-b]pyridin-7-ium

According to the general procedure for N-alkylation of amides (Method B). Methyl 5-(2-methyl-6-{thieno[2,3-b]pyridine-2-amido}phenyl)thiophene-2-carboxylate (100 mg, 0.24 mmol), DMF (1 mL). Purification using preparative HPLC Method A. Solvent was concentrated under vacuo and lyophilization to give the desired pyridine product as a pink solid (69.5 mg, 67% yield). LCMS (220 nm, 254 nm): t_{R} 2.717 min, purity ≥95%, m/z (ESI): 423.20 [M+H]⁺. and the pyridinium product as a clear oil (51.7 mg, 48% yield). LCMS of pyridinium side product (220 nm, 254 nm): t_{R} 2.145 min, purity ≥95%, m/z (ESI): 438.30 [M+H]⁺.

### Synthesis of 5-(2-methyl-6-{N-methylthieno[2,3-b]pyridine-2-amido}phenyl)thiophene-2-carboxylic acid (RU-NT-478)

According to the general procedure for saponification of methyl esters. Methyl 5-(2-methyl-6-{N-methylthieno[2,3-b]pyridine-2-amido}phenyl)thiophene-2-carboxylate (60 mg, 0.14 mmol). Purification using preparative HPLC Method A. Solvent was concentrated under vacuo and lyophilization to give the desired product as a white solid (13 mg, 22% yield). LCMS (220 nm, 254 nm): t_{R} 2.329 min, purity ≥95%, m/z (EI): 408.20 [M]⁺. ¹H NMR (500 MHz, DMSO-d₆) δ 8.58 (dd, J=4.7, 1.5 Hz, 1H), 8.38-8.04 (m, 1H), 7.63-7.31 (m, 3H), 7.25 (s, 1H), 7.15 (s, 1H), 6.67 (d, J=3.6 Hz, 1H), 3.11 (s, 3H), 2.20 (s, 3H).

### Synthesis of 2-{[2-(5-carboxythiophen-2-yl)-3-methylphenyl](methyl)carbamoyl}-7-methylthieno[2,3-b]pyridin-7-ium (RU-NT-479)

According to the general procedure for saponification of methyl esters. 2-({2-[5-(Methoxycarbonyl)thiophen-2-yl]-3-methylphenyl}(methyl)carbamoyl)-7-methylthieno[2,3-b]pyridin-7-ium (51.7 mg, 0.12 mmol). Purification using preparative HPLC Method A. Solvent was concentrated under vacuo and lyophilization to give the desired product as a white solid (20 mg, 40% yield). LCMS (220 nm, 254 nm, Rt=2.175 min), m/z: 424.25 [M+H, 100%]⁺. ¹H NMR (500 MHz, DMSO-d₆) δ 8.20 (s, 1H), 7.68 (d, J=9.4 Hz, 1H), 7.50 (d, J=7.9 Hz, 2H), 7.40 (d, J=7.7 Hz, 2H), 6.77 (s, 1H), 6.47-6.18 (m, 2H), 3.47 (s, 3H), 3.07 (s, 3H), 2.21 (s, 3H).

### Synthesis of N-(2-bromo-3-methylphenyl)thieno[3,2-b]pyridine-2-carboxamide

According to general procedure C for the amide coupling using T3P. Thieno[3,2-b]pyridine-2-carboxylic acid (250 mg, 1.4 mmol) was added 2-bromo-3-methylaniline (311.49 mg, 1.67 mmol), EtOAc (3 mL) and DCM (2 mL). Purification using preparative HPLC Method A. Solvent was concentrated under vacuo and lyophilization to give the desired product as a brownish solid (480 mg, 99% yield). LCMS (220 nm, 254 nm): t_{R} 2.187 min, purity ≥95%, m/z (ESI): 387.15 [M+H]⁺.

### Synthesis of methyl 5-(2-methyl-6-{thieno[3,2-b]pyridine-2-amido}phenyl)thiophene-2-carboxylate

According to the general procedure for Suzuki coupling reactions using Method A, precatalyst C1. N-(2-bromo-3-methylphenyl)thieno[3,2-b]pyridine-2-carboxamide (300 mg, 0.86 mmol), [5-(methoxycarbonyl)thiophen-2-yl]boronic acid (192 mg, 1.04 mmol). Purification using preparative HPLC Method A. Solvent was concentrated under vacuo and lyophilization to give the desired product as a white solid (130 mg, 37% yield). LCMS (220 nm, 254 nm): t_{R} 2.371 min, purity ≥95%, m/z (ESI): 410.20 [M+H]⁺.

### Synthesis of 5-(2-methyl-6-{thieno[3,2-b]pyridine-2-amido}phenyl)thiophene-2-carboxylic acid (RU-NT-480)

According to the general procedure for saponification of methyl esters. Methyl 5-(2-methyl-6-{thieno[3,2-b]pyridine-2-amido}phenyl)thiophene-2-carboxylate (30 mg, 0.07 mmol). Purification using preparative HPLC Method A. Solvent was concentrated under vacuo and lyophilization to give the desired product as a white solid (15 mg, 52% yield). LCMS (220 nm, 254 nm): t_{R} 2.017 min, purity ≥95%, m/z (ESI): 395.25 [M+H]⁺. ¹H NMR (500 MHz, DMSO-d₆) δ 9.90 (br s, 1H), 8.71 (dd, J=4.6, 1.5 Hz, 1H), 8.51 (d, J=8.2 Hz, 1H), 8.29 (s, 1H), 8.06 (s, 1H), 7.48-7.32 (m, 3H), 7.29 (d, J=7.7 Hz, 2H), 6.91 (d, J=3.5 Hz, 1H), 2.23 (s, 3H).

### Synthesis of 2-({2-[5-(methoxycarbonyl)thiophen-2-yl]-3-methylphenyl}(methyl)carbamoyl)-4-methylthieno[3,2-b]pyridin-4-ium (RU-NT-481)

According to the general procedure for saponification of methyl esters. 2-({2-[5-(Methoxycarbonyl)thiophen-2-yl]-3-methylphenyl}(methyl)carbamoyl)-4-methylthieno[3,2-b]pyridin-4-ium (90 mg, 0.21 mmol). Purification using preparative HPLC Method A. Solvent was concentrated under vacuo and lyophilization to give the desired product as a white solid (6 mg, 7% yield). LCMS (220 nm, 254 nm): t_{R} 1.831 min, purity ≥95%, m/z (ESI): 424.30 [M+H]⁺. ¹H NMR (500 MHz, DMSO-d₆) δ 9.21 (d, J=8.3 Hz, 1H), 9.13 (d, J=5.9 Hz, 1H), 8.35 (s, 1H), 8.02 (dd, J=8.4, 5.9 Hz, 1H), 7.83 (d, J=0.8 Hz, 1H), 7.54 (dd, J=6.0, 3.2 Hz, 1H), 7.51-7.43 (m, 2H), 7.10 (d, J=3.5 Hz, 1H), 6.64 (d, J=3.5 Hz, 1H), 4.42 (s, 3H), 3.19 (s, 3H), 2.22 (s, 3H).

### Synthesis of methyl 5-(2-methyl-6-{N-methylthieno[3,2-b]pyridine-2-amido}phenyl)thiophene-2-carboxylate & 2-({2-[5-(methoxycarbonyl)thiophen-2-yl]-3-methylphenyl}(methyl)carbamoyl)-4-methylthieno[3,2-b]pyridin-4-ium

According to the general procedure for N-alkylation of amides (Method B). Methyl 5-(2-methyl-6-{thieno[3,2-b]pyridine-2-amido}phenyl)thiophene-2-carboxylate (100 mg, 0.24 mmol), DMF (1 mL). Purification using preparative HPLC Method A. Solvent was concentrated under vacuo and lyophilization to give the desired pyridine product as a clear oil (21.9 mg, 21% yield). LCMS (220 nm, 254 nm): t_{R} 3.021 min, purity ≥95%, m/z (EI): 338.20 [M]⁺. and the pyridinium product as a clear oil. LCMS (220 nm, 254 nm): t_{R} 3.021 min, purity ≥95%, m/z (EI): 437.35 [M]⁺.

### Synthesis of 2-{[2-(5-carboxythiophen-2-yl)-3-methylphenyl](methyl)carbamoyl}-4-methylthieno[3,2-b]pyridin-4-ium (RU-NT-482)

According to the general procedure for saponification of methyl esters. 2-({2-[5-(Methoxycarbonyl)thiophen-2-yl]-3-methylphenyl}(methyl)carbamoyl)-4-methylthieno[3,2-b]pyridin-4-ium (90 mg, 0.21 mmol). Purification using preparative HPLC Method A. Solvent was concentrated under vacuo and lyophilization to give the desired product as a white solid (6 mg, 7% yield). LCMS (220 nm, 254 nm): t_{R} 1.831 min, purity ≥95%, m/z (ESI): 424.30 [M+H]⁺. ¹H NMR (500 MHz, DMSO-d₆) δ 9.21 (d, J=8.3 Hz, 1H), 9.13 (d, J=5.9 Hz, 1H), 8.35 (s, 1H), 8.02 (dd, J=8.4, 5.9 Hz, 1H), 7.83 (d, J=0.8 Hz, 1H), 7.54 (dd, J=6.0, 3.2 Hz, 1H), 7.51-7.43 (m, 2H), 7.10 (d, J=3.5 Hz, 1H), 6.64 (d, J=3.5 Hz, 1H), 4.42 (s, 3H), 3.19 (s, 3H), 2.22 (s, 3H).

### Synthesis of N-(2-bromo-3-methylphenyl)-3-methylthiophene-2-sulfonamide

To a stirred solution of 3-methylthiophene-2-sulfonyl chloride (250 mg, 1.27 mmol) in DCM (2 mL) in a 20 mL scintillation vial was added 2-bromo-3-methylaniline (260 mg, quantitative yield assumed from previous step) and excess TEA (3 eq) at room temperature. The reaction mixture was left under stirring at rtp O/N then diluted with dichloromethane and washed with a saturated aqueous solution of brine and then dried over anhydrous MgSO₄. The solution was concentrated under reduced pressure, and the crude material was fractionated and purified. Purification using preparative HPLC Method B. Solvent was concentrated under vacuo and lyophilization to give the desired product as a white solid (110 mg, 18% yield). LCMS (220 nm, 254 nm): t_{R} 2.737 min, purity ≥95%, m/z (ESI): 347.00 [M+H]⁺.

### Synthesis of methyl 5-[2-methyl-6-(3-methylthiophene-2-sulfonamido)phenyl]thiophene-2-carboxylate

According to the general procedure for Suzuki coupling reactions using Method A, precatalyst C1. N-(2-bromo-3-methylphenyl)-3-methylthiophene-2-sulfonamide (78.6 mg, 0.23 mmol), [5-(methoxycarbonyl)thiophen-2-yl]boronic acid (54.88 mg, 0.3 mmol). Purification using preparative HPLC Method B. Solvent was concentrated under vacuo and lyophilization to give the desired product as a white solid (45 mg, 49% yield). LCMS (220 nm, 254 nm): t_{R} 2.529 min, purity ≥95%, m/z (ESI): 431.20 [M+CH₃CN]⁺. ¹H NMR (500 MHz, MeOD-d₄) δ 8.31 (dd, J=3.8, 1.5 Hz, 1H), 8.15 (d, J=4.9 Hz, 1H), 8.00-7.92 (m, 1H), 7.90 (d, J=7.9 Hz, 1H), 7.80 (d, J=7.7 Hz, 1H), 7.54 (d, J=5.0 Hz, 1H), 7.19 (dd, J=3.8, 1.5 Hz, 1H), 2.70 (s, 3H), 2.69 (s, 3H).

### Synthesis of 5-[2-methyl-6-(3-methylthiophene-2-sulfonamido)phenyl]thiophene-2-carboxylic acid (RU-NT-483)

According to the general procedure for saponification of methyl esters. Methyl 5-[2-methyl-6-(3-methylthiophene-2-sulfonamido)phenyl]thiophene-2-carboxylate (10 mg, 0.02 mmol). Purification using preparative HPLC Method B. Solvent was concentrated under vacuo and lyophilization to give the desired product as a white solid (7.7 mg, 100% yield). LCMS (220 nm, 254 nm): t_{R} 2.535 min, purity ≥95%, m/z (ESI): 416.20 [M+Na, 100%]⁺. ¹H NMR (500 MHz, DMSO-d₆) δ 13.06 (br s, 1H), 9.53 (s, 1H), 7.72 (d, J=5.0 Hz, 1H), 7.65 (d, J=3.6 Hz, 1H), 7.30 (t, J=7.8 Hz, 1H), 7.24 (d, J=7.6 Hz, 1H), 7.03 (d, J=7.9 Hz, 1H), 6.98 (d, J=5.0 Hz, 1H), 6.69 (d, J=3.7 Hz, 1H), 2.11 (s, 3H), 2.08 (s, 3H).

### Synthesis of methyl 5-[2-methyl-6-(N-methyl-3-methylthiophene-2-sulfonamido)phenyl]thiophene-2-carboxylate

According to the general procedure for N-alkylation of amides (Method B). Methyl 5-[2-methyl-6-(3-methylthiophene-2-sulfonamido)phenyl]thiophene-2-carboxylate (35 mg, 0.09 mmol). Purification using preparative HPLC Method B. Solvent was concentrated under vacuo and to give the desired product as a white solid (11 mg, 30% yield). LCMS (220 nm, 254 nm): t_{R} 3.070 min, purity ≥95%, m/z (ESI): 485.15 [M+CH₃CN+Na]⁺.

### Synthesis of methyl 5-[2-methyl-6-(N-methyl-3-methylthiophene-2-sulfonamido)phenyl]thiophene-2-carboxylate (RU-NT-484)

According to the general procedure for saponification of methyl esters. Methyl 5-[2-methyl-6-(N-methyl-3-methylthiophene-2-sulfonamido)phenyl]thiophene-2-carboxylate (11 mg, 0.03 mmol). Purification using preparative HPLC Method B. Solvent was concentrated under vacuo and to give the desired product as a white solid white solid (6 mg, 60% yield). LCMS (220 nm, 254 nm): t_{R} 2.803 min, purity ≥95%, m/z (ESI): 471.15 [M+CH₃CN+Na]⁺. ¹H NMR (500 MHz, DMSO-d₆) δ 13.12 (br s, 1H), 7.91 (d, J=5.0 Hz, 1H), 7.74 (d, J=3.7 Hz, 1H), 7.39 (d, J=7.5 Hz, 1H), 7.34 (t, J=7.7 Hz, 1H), 7.10 (d, J=5.1 Hz, 1H), 7.04 (d, J=3.7 Hz, 1H), 6.79 (d, J=7.7 Hz, 1H), 3.04 (s, 3H), 2.17 (s, 3H), 2.16 (s, 3H).

### Synthesis of N-(2-bromo-3-methylphenyl)-7-fluoro-1-benzothiophene-2-carboxamide

According to general procedure C for the amide coupling using T3P. 7-Fluoro-1-benzothiophene-2-carboxylic acid (300 mg, 1.53 mmol), 2-bromo-3-methylaniline (341 mg, 1.83 mmol), EtOAc (3 mL) and DCM (1 mL). Purification using preparative HPLC Method B. Solvent was concentrated under vacuo and to give the desired product as a white solid (0.13 g, 23% yield). LCMS (220 nm, 254 nm): t_{R} 3.227 min, purity ≥95%, m/z (ESI): 366.10 [M+H, 100%]⁺. ¹H NMR (500 MHz, DMSO-d₆) δ 10.47 (s, 1H), 8.43 (d, J=3.8 Hz, 1H), 7.90 (d, J=7.9 Hz, 1H), 7.53 (dt, J=8.2, 4.1 Hz, 1H), 7.50-6.94 (m, 4H), 2.44 (s, 3H).

### Synthesis of methyl 5-[2-(7-fluoro-1-benzothiophene-2-amido)-6-methylphenyl]thiophene-2-carboxylate

According to the general procedure for Suzuki coupling reactions using Method A, precatalyst C1. N-(2-bromo-3-methylphenyl)-7-fluoro-1-benzothiophene-2-carboxamide (100 mg, 0.27 mmol), [5-(methoxycarbonyl)thiophen-2-yl]boronic acid (61 mg, 0.33 mmol). Purification using preparative HPLC Method B. Solvent was concentrated under vacuo and to give the desired product as a beige solid (25 mg, 21% yield). LCMS (220 nm, 254 nm): t_{R} 3.173 min, purity ≥95%, m/z (ESI): 426.15 [M+H]⁺.

### Synthesis of 5-[2-(7-fluoro-]-benzothiophene-2-amido)-6-methylphenyl]thiophene-2-carboxylic acid (RU-NT-485)

According to the general procedure for saponification of methyl esters. Methyl 5-[2-(7-fluoro-1-benzothiophene-2-amido)-6-methylphenyl]thiophene-2-carboxylate (5 mg, 0.01 mmol). Purification using preparative HPLC Method B. Solvent was concentrated under vacuo and to give the desired product as a white solid (4.04 mg, 84% yield). LCMS (220 nm, 254 nm): t_{R} 2.740 min, purity ≥95%, m/z (ESI): 412.05 [M+H]⁺. ¹H NMR (500 MHz, DMSO-d₆) δ 13.05 (brs, 1H), 10.16 (s, 1H), 8.09 (d, J=3.6 Hz, 1H), 7.81 (d, J=8.0 Hz, 1H), 7.68 (d, J=3.8 Hz, 1H), 7.48 (td, J=8.0, 5.1 Hz, 1H), 7.43 (t, J=7.7 Hz, 1H), 7.39-7.28 (m, 3H), 7.08 (d, J=3.8 Hz, 1H), 2.23 (s, 3H). ¹⁹F NMR (471 MHz, DMSO-d₆) δ-115.36 Hz.

### Synthesis of methyl 5-[2-methyl-6-(N-methyl-7-fluoro-]-benzothiophene-2-amido)phenyl]thiophene-2-carboxylate

According to the general procedure for N-alkylation of amides (Method B). Methyl 5-[2-(7-fluoro-1-benzothiophene-2-amido)-6-methylphenyl]thiophene-2-carboxylate (20 mg, 0.05 mmol), DMF (1 mL). Purification using preparative HPLC Method B. Solvent was concentrated under vacuo and to give the desired product as a white solid (20 mg, 97% yield). LCMS (220 nm, 254 nm): t_{R} 3.203 min, purity ≥95%, m/z (ESI): 440.15 [M+H]⁺.

### Synthesis of 5-[2-methyl-6-(N-methyl-7-fluoro-1-benzothiophene-2-amido)phenyl]thiophene-2-carboxylic acid (RU-NT-486)

According to the general procedure for saponification of methyl esters. Methyl-5-[2-methyl-6-(N-methyl-7-fluoro-1-benzothiophene-2-amido)phenyl]thiophene-2-carboxylate (20 mg, 0.05 mmol). Purification using preparative HPLC Method B. Solvent was concentrated under vacuo and to give the desired product as a white solid (17.73 mg, 92% yield). LCMS (220 nm, 254 nm): t_{R} 2.937 min, purity ≥95%, m/z (ESI): 426.15 [M+H]⁺. ¹H NMR (500 MHz, DMSO-d₆) δ 13.18 (br s, 1H), 7.68 (d, J=3.8 Hz, 1H), 7.63 (d, J=8.0 Hz, 1H), 7.59-7.52 (m, 2H), 7.52-7.47 (m, 1H), 7.39 (td, J=7.9, 5.0 Hz, 1H), 7.30 (dd, J=10.3, 7.8 Hz, 1H), 7.03 (d, J=3.6 Hz, 1H), 6.86 (d, J=3.8 Hz, 1H), 3.13 (s, 3H), 2.19 (s, 3H). ¹⁹F NMR (471 MHz, DMSO-d₆) δ-115.94 Hz. LCMS (220 nm, 254 nm): t_{R} 2.937 min, purity ≥95%, m/z (ESI): 426.15 [M+H]⁺.

### Synthesis of 3,5-dimethyl-1-benzothiophene-2-carboxylic acid

According to the general procedure for saponification of methyl esters. Methyl 3,5-dimethyl-1-benzothiophene-2-carboxylate (0.8 g, 3.63 mmol). After acidification to a pH of 2-3 with 2M HCl, extraction was carried out 3x with EtOAc and brine and the combined organic layers were dried over anhydrous MgSO₄. The desired product was obtained as a white solid (0.41 g, 55% yield). LCMS (220 nm, 254 nm): t_{R} 2.463 min, purity ≥95%, m/z (ESI): 248.35 [M+CH₃CN]⁺.

### Synthesis of N-(2-bromo-3-methylphenyl)-3,5-dimethyl-1-benzothiophene-2-carboxamide

According to general procedure B for amide coupling. 3,5-Dimethyl-1-benzothiophene-2-carboxylic acid (0.4 g, 1.94 mmol), 2-bromo-3-methylaniline, DMF (3 mL). Purification using preparative HPLC Method B. Solvent was concentrated under vacuo and to give the desired product as an off-white solid (40 mg, 5% yield). LCMS (220 nm, 254 nm): t_{R} 3.677 min, purity ≥95%, m/z (ESI): 374.15 [M+H]⁺. ¹H NMR (500 MHz, DMSO-d₆) δ 9.79 (s, 1H), 7.91 (d, J=8.2 Hz, 1H), 7.74 (s, 1H), 7.51-7.45 (m, 1H), 7.40-7.34 (m, 1H), 7.32 (d, J=7.7 Hz, 1H), 7.28 (d, J=7.4 Hz, 1H), 2.70 (s, 3H), 2.48 (s, 3H), 2.43 (s, 3H).

### Synthesis of methyl 5-[2-(3,5-dimethyl-]-benzothiophene-2-amido)-6-methylphenyl]thiophene-2-carboxylate & 5-[2-(3,5-dimethyl-]-benzothiophene-2-amido)-6-methylphenyl]thiophene-2-carboxylic acid (RU-NT-487)

According to the general procedure for Suzuki coupling reactions using Method A, precatalyst C1. N-(2-Bromo-3-methylphenyl)-3,5-dimethyl-1-benzothiophene-2-carboxamide (40 mg, 0.11 mmol), [5-(methoxycarbonyl)thiophen-2-yl]boronic acid (31.8 mg, 0.17 mmol), dry dioxane (1 mL), temperature: 100° C. Purification using preparative HPLC Method B. Solvent was concentrated under vacuo and to give the desired product as a white solid (25.5 mg, 55% yield). LCMS (220 nm, 254 nm): t_{R} 3.493 min, purity ≥95%, m/z (ESI): 436.25 [M+H]⁺. t_{R} 3.493 min, purity ≥95%, m/z (ESI): 436.25 [M+H]⁺ and the acid side product as an off-white solid (7.15 mg, 16% yield). LCMS (220 nm, 254 nm): t_{R} 3.493 min, purity ≥95%, m/z (ESI): 436.25 [M+H]⁺. ¹H NMR (500 MHz, DMSO-d₆) δ 13.10 (br s, 1H), 9.38 (s, 1H), 7.84 (d, J=8.2 Hz, 1H), 7.75 (d, J=3.7 Hz, 1H), 7.66 (s, 1H), 7.50 (d, J=7.9 Hz, 1H), 7.42 (t, J=7.8 Hz, 1H), 7.30 (d, J=7.9 Hz, 1H), 7.10 (d, J=3.7 Hz, 1H), 2.44 (s, 3H), 2.32 (s, 3H), 2.19 (s, 3H).

### Synthesis of methyl 3,5-dimethyl-]-benzothiophene-2-carboxylate

According to the general procedure for the synthesis of substituted benzothiophenes. Methyl 2-mercaptoacetate (4.1 g, 39.4 mmol), 1-(2-fluoro-5-methylphenyl)ethan-1-one (5.0 g, 32.8 mmol).

Purification using preparative HPLC Method B. Solvent was concentrated under vacuo and to give the desired product as white crystals (1.09 g, 15% yield). LCMS (220 nm, 254 nm): t_{R} 3.077 min, purity ≥95%, m/z (ESI): 262.30 [M+H]⁺. ¹H NMR (500 MHz, DMSO-d₆) δ 7.86 (d, J=8.2 Hz, 1H), 7.74 (s, 1H), 7.37 (dd, J=8.3, 1.6 Hz, 1H), 3.85 (s, 3H), 2.68 (s, 3H), 2.44 (s, 3H).

### Synthesis of 3,5-dimethyl-]-benzothiophene-2-carboxylic acid

According to the general procedure for saponification of methyl esters. Methyl 3,5-dimethyl-1-benzothiophene-2-carboxylate (0.8 g, 3.63 mmol). Purification using preparative HPLC Method B. Solvent was concentrated under vacuo and to give the desired product as a white solid (0.41 g, 55% yield). LCMS (220 nm, 254 nm): t_{R} 2.463 min, purity ≥95%, m/z (ESI): 286.20 [M+CH₃CN]⁺.

### Synthesis of N-(2-bromo-3-methylphenyl)-3,5-dimethyl-1-benzothiophene-2-carboxamide

According to general procedure B for the amide coupling. EDC HCl (0.56 g, 2.91 mmol), HOAt (0.4 g, 2.91 mmol), 3,5-dimethyl-1-benzothiophene-2-carboxylic acid (0.4 g, 1.94 mmol), 2-bromo-3-methylaniline (1.1 eq), DMF (3 mL). Purification using preparative HPLC Method B. Solvent was concentrated under vacuo and to give the desired product as an off-white solid (40 mg, 5% yield). LCMS (220 nm, 254 nm): t_{R} 3.677 min, purity ≥95%, m/z (ESI): 374.15 [M+H, 100%]⁺. ¹H NMR (500 MHz, DMSO-d₆) δ 9.79 (s, 1H), 7.91 (d, J=8.2 Hz, 1H), 7.74 (s, 1H), 7.51-7.45 (m, 1H), 7.40-7.34 (m, 1H), 7.32 (d, J=7.7 Hz, 1H), 7.28 (d, J=7.4 Hz, 1H), 2.70 (s, 3H), 2.48 (s, 3H), 2.43 (s, 3H).

### Synthesis of methyl 5-[2-(3,5-dimethyl-]-benzothiophene-2-amido)-6-methylphenyl]thiophene-2-carboxylate

According to the general procedure for Suzuki coupling reactions using Method A, precatalyst C1. N-(2-bromo-3-methylphenyl)-3,5-dimethyl-1-benzothiophene-2-carboxamide (40 mg, 0.11 mmol), [5-(methoxycarbonyl)thiophen-2-yl]boronic acid (31.8 mg, 0.17 mmol). Purification using preparative HPLC Method B. Solvent was concentrated under vacuo and to give the desired product as a white solid (25.5 mg, 55% yield). LCMS (220 nm, 254 nm): t_{R} 3.493 min, purity ≥95%, m/z (ESI): 436.25 [M+H]⁺.

### Synthesis of 5-[2-methyl-6-(N-methyl-3,5-dimethyl-]-benzothiophene-2-amido)phenyl]thiophene-2-carboxylic acid (RU-NT-488)

According to the general procedure for saponification of methyl esters. Methyl 5-[2-methyl-6-(N-methyl-3,5-dimethyl-1-benzothiophene-2-amido)phenyl]thiophene-2-carboxylate (19.9 mg, 0.04 mmol). Purification using preparative HPLC Method B. Solvent was concentrated under vacuo and to give the desired product as a white solid (0.13 g, 23% yield). LCMS (220 nm, 254 nm): t_{R} 3.227 min, purity ≥95%, m/z (ESI): 366.10 [M+H]⁺. ¹H NMR for the observed rotamers (500 MHz, DMSO-d₆) δ 13.18 (br s, 1H), 7.90-7.75 (m, 1H), 7.73 (d, J=3.8 Hz, 1H), 7.69-7.45 (m, 2H), 7.44-7.24 (m, 2H), 7.24-7.06 (m, 1H), 6.84 (d, J=3.8 Hz, 1H), 3.08 (d, J=30.2 Hz, 3H), 2.41 (s, 3H), 2.24 (d, J=63.0 Hz, 3H), 2.08 (d, J=27.8 Hz, 3H).

### Synthesis of methyl 5-phenylthiophene-2-carboxylate

According to the general procedure for Suzuki coupling reactions using Method B. Methyl 5-bromothiophene-2-carboxylate (0.66 g, 3 mmol), phenylboronic acid (1.5 mmol), toluene/ethanol/water (10:3:2, v/v/v, 10 mL). Temperature: 100° C. Purification using preparative HPLC Method B. Solvent was concentrated under vacuo and to give the desired product as a white solid (0.15 g, 23% yield). LCMS (220 nm, 254 nm): t_{R} 2.837 min, purity ≥95%, m/z (ESI): 219.35 [M+H]⁺.

### Synthesis of 5-phenylthiophene-2-carboxylic acid

According to the general procedure for saponification of methyl esters. Methyl 5-phenylthiophene-2-carboxylate (0.12 g, 0.53 mmol). Workup similar to that in the general procedure was carried out for the purification to afford a white solid (0.1 g, 93% yield). LCMS (220 nm, 254 nm): t_{R} 2.285 min, purity ≥95%, m/z (ESI): 243.50[M+K]⁺.

### Synthesis of N-(2-bromo-3-methylphenyl)-5-phenylthiophene-2-carboxamide

According to general procedure C for the amide coupling using T3P. 5-Phenylthiophene-2-carboxylic acid (0.11 g, 0.54 mmol), 2-bromo-3-methylaniline (0.12 g, 0.65 mmol), EtOAc (5 mL). Purification using preparative HPLC Method B. Solvent was concentrated under vacuo and to give the desired product as a white solid (56 mg, 28% yield). LCMS (220 nm, 254 nm): t_{R} 3.332 min, purity ≥95%, m/z (ESI): 373.55 [M+H]⁺. ¹H NMR (500 MHz, DMSO-d₆) δ 10.08 (s, 1H), 8.00 (d, J=3.9 Hz, 1H), 7.84-7.70 (m, 2H), 7.62 (d, J=3.9 Hz, 1H), 7.47 (t, J=7.6 Hz, 2H), 7.43-7.17 (m, 4H), 2.42 (s, 3H).

### Synthesis of methyl 5-[2-methyl-6-(5-phenylthiophene-2-amido)phenyl]thiophene-2-carboxylate

According to the general procedure for Suzuki coupling reactions using Method A, precatalyst C1. N-(2-bromo-3-methylphenyl)-5-phenylthiophene-2-carboxamide (50 mg, 0.13 mmol), [5-(methoxycarbonyl)thiophen-2-yl]boronic acid (29.98 mg, 0.16 mmol). Temperature: 100° C. Purification using preparative HPLC Method B. Solvent was concentrated under vacuo and to give the desired product as a white solid for the ester (11.1 mg, 19% yield). LCMS (220 nm, 254 nm): t_{R} 3.260 min, purity ≥95%, m/z (ESI): 434.30 [M+H]⁺. ¹H NMR (500 MHz, DMSO-d₆) δ 9.75 (s, 1H), 7.79 (d, J=3.8 Hz, 1H), 7.72-7.67 (m, 2H), 7.64 (d, J=4.0 Hz, 1H), 7.52 (d, J=3.9 Hz, 1H), 7.48-7.42 (m, 2H), 7.41 (d, J=7.7 Hz, 1H), 7.39-7.36 (m, 1H), 7.36-7.33 (m, 1H), 7.34-7.27 (m, 1H), 7.11 (d, J=3.8 Hz, 1H), 3.79 (s, 3H), 2.54 (s, 3H).

### Synthesis of 5-[2-methyl-6-(5-phenylthiophene-2-amido)phenyl]thiophene-2-carboxylic acid (RU-NT-489)

According to the general procedure for saponification of methyl esters. Methyl 5-[2-methyl-6-(5-phenylthiophene-2-amido)phenyl]thiophene-2-carboxylate (11.1 mg, 0.03 mmol). Purification using preparative HPLC Method B. Solvent was concentrated under vacuo and to give the desired product as a white solid (6 mg, 56% yield). LCMS (220 nm, 254 nm): t_{R} 2.837 min, purity ≥95%, m/z (ESI): 420.25 [M+H]⁺. ¹H NMR (500 MHz, DMSO-d₆) δ 13.08 (br s, 1H), 9.72 (s, 1H), 7.76-7.67 (m, 1H), 7.63 (d, J=3.9 Hz, 3H), 7.52 (d, J=3.9 Hz, 1H), 7.44 (t, J=7.6 Hz, 1H), 7.41-7.37 (m, 1H), 7.35 (t, J=8.4 Hz, 2H), 7.31 (d, J=7.6 Hz, 1H), 7.08 (d, J=3.8 Hz, 2H), 2.22 (s, 3H).

### Synthesis of methyl 5-[2-methyl-6-(N-methyl-5-phenylthiophene-2-amido)phenyl]thiophene-2-carboxylate

According to the general procedure for N-alkylation of amides (Method B). 5-[2-Methyl-6-(5-phenylthiophene-2-amido)phenyl]thiophene-2-carboxylic acid (12.8 mg, 0.03 mmol), DMF (1 mL). Purification using preparative HPLC Method B. Solvent was concentrated under vacuo and to give the desired product as a colorless oil (12.3 mg, 90% yield). LCMS (220 nm, 254 nm): t_{R} 3.283 min, purity ≥95%, m/z (ESI): 448.30 [M+H]⁺.

### Synthesis of 5-[2-methyl-6-(N-methyl-5-phenylthiophene-2-amido)phenyl]thiophene-2-carboxylic acid (RU-NT-490)

According to the general procedure for saponification of methyl esters. Methyl 5-[2-methyl-6-(N-methyl-5-phenylthiophene-2-amido)phenyl]thiophene-2-carboxylate (12.3 mg, 0.03 mmol). Purification using preparative HPLC Method B. Solvent was concentrated under vacuo and to give the desired product as a white solid was collected (2.77 mg, 23% yield). LCMS (220 nm, 254 nm): t_{R} 2.880 min, purity ≥95%, m/z (ESI): 434.25 [M+H]⁺. ¹H NMR (500 MHz, DMSO-d₆) δ 13.17 (br s, 1H), 7.69 (d, J=3.7 Hz, 1H), 7.58 (d, J=7.6 Hz, 1H), 7.52 (d, J=5.8 Hz, 1H), 7.42 (q, J=6.0 Hz, 2H), 7.35 (t, J=7.3 Hz, 1H), 7.30 (d, J=4.0 Hz, 1H), 6.88 (d, J=3.7 Hz, 1H), 6.39 (d, J=4.0 Hz, 1H), 3.08 (s, 3H), 2.20 (s, 3H).

### Synthesis of N-(3-bromo-2-methylpyridin-4-yl)-3-methylthiophene-2-carboxamide

According to the general procedure for N-alkylation of amides (Method B). 3-Methylthiophene-2-carboxylic acid (209 mg, 1.47 mmol), 3-bromo-2-methylpyridin-4-amine (250 mg, 1.34 mmol), EtOAc (3 mL). The crude was taken directly to the next step. LCMS (220 nm, 254 nm): t_{R} 1.687 min, purity ≥95%, m/z (ESI): 312.90 [M+H]⁺.

### Synthesis of methyl 5-[4-methyl-2-(3-methylthiophene-2-amido)pyridin-3-yl]thiophene-2-carboxylate

According to the general procedure for Suzuki coupling reactions using Method A, precatalyst C1. N-(3-bromo-4-methylpyridin-2-yl)-3-methylthiophene-2-carboxamide (200 mg, 0.64 mmol), [5-(methoxycarbonyl)thiophen-2-yl]boronic acid (143 mg, 0.64 mmol). Purification was carried out only via extraction according to the general procedure before being taken directly to the next step. Yellow oil (33 mg, 14% yield). LCMS (220 nm, 254 nm): t_{R} 2.028 min, purity ≥95%, m/z (ESI): 373.20 [M+H]⁺.

### Synthesis of methyl 5-[4-methyl-2-(N-methyl-3-methylthiophene-2-amido)pyridin-3-yl]thiophene-2-carboxylate

According to the general procedure for N-alkylation of amides (Method B). Methyl 5-[4-methyl-2-(3-methylthiophene-2-amido)pyridin-3-yl]thiophene-2-carboxylate (25 mg, 0.07 mmol). Purification using preparative HPLC Method B. Solvent was concentrated under vacuo and to give the desired product as a clear oil (10 mg, 39% yield). LCMS (220 nm, 254 nm): t_{R} 2.415 min, purity ≥95%, m/z (ESI): 387.25 [M+H]⁺.

### Synthesis of 5-[4-methyl-2-(N-methyl-3-methylthiophene-2-amido)pyridin-3-yl]thiophene-2-carboxylic acid (RU-NT-491)

According to the general procedure for saponification of methyl esters. Methyl 5-[4-methyl-2-(N-methyl-3-methylthiophene-2-amido)pyridin-3-yl]thiophene-2-carboxylate (10 mg, 0.03 mmol). Crude mixture was reduced under vacuum after acidification with 2M HCl to pKa of 5-6. Purification using preparative HPLC Method A. Solvent was concentrated under vacuo and the sample was lyophilized to give the desired product as a white solid (4.7 mg, 49% yield). LCMS (220 nm, 254 nm): t_{R} 1.980 min, purity ≥95%, m/z (ESI): 373.30 [M+H]⁺. ¹H NMR (500 MHz, Acetone-d₆) δ 8.44 (d, J=5.0 Hz, 1H), 7.76 (d, J=3.8 Hz, 1H), 7.39 (dd, J=5.0, 0.8 Hz, 1H), 7.31 (d, J=5.0 Hz, 1H), 6.80 (d, J=4.6 Hz, 2H), 3.26 (s, 3H), 2.23 (d, J=0.6 Hz, 3H), 2.14 (s, 3H).

### Synthesis of N-(2-bromo-3-methylphenyl)thieno[2,3-c]pyridine-2-carboxamide

According to general procedure C for the amide coupling using T3P. Thieno[2,3-c]pyridine-2-carboxylic acid (250 mg, 1.4 mmol), 2-bromo-3-methylaniline (311.49 mg, 1.67 mmol). Purification using preparative HPLC Method A. Solvent was concentrated under vacuo and the sample was lyophilized to give the desired product as a white solid (246 mg, 51% yield). LCMS (220 nm, 254 nm): t_{R} 1.799 min, purity ≥95%, m/z (ESI): 348.20 [M+H]⁺.

### Synthesis of methyl 5-(2-methyl-6-{thieno[2,3-c]pyridine-2-amido}phenyl)thiophene-2-carboxylate

According to the general procedure for Suzuki coupling reactions using Method A, precatalyst C1. N-(2-bromo-3-methylphenyl)thieno[2,3-c]pyridine-2-carboxamide (200 mg, 0.58 mmol), [5-(methoxycarbonyl)thiophen-2-yl]boronic acid (128 mg, 0.69 mmol). Purification using preparative HPLC Method A. Solvent was concentrated under vacuo and the sample was lyophilized to give the desired product as a beige solid (74.4 mg, 32% yield). LCMS (220 nm, 254 nm): t_{R} 2.127 min, purity ≥95%, m/z (ESI): 409.10 [M+H]⁺.

### Synthesis of 2-{[2-(5-carboxythiophen-2-yl)-3-methylphenyl](methyl)carbamoyl}-6-methylthieno[2,3-c]pyridin-6-ium (RU-NT-492)

According to the general procedure for N-alkylation of amides (Method B). Methyl 5-(2-methyl-6-{thieno[2,3-c]pyridine-2-amido}phenyl)thiophene-2-carboxylate (60 mg, 0.18 mmol). Purification using preparative HPLC Method A. Solvent was concentrated under vacuo and the sample lyophilized to give the desired product as a white solid (13.5 mg, 18% yield). LCMS (220 nm, 254 nm): t_{R} 1.813 min, purity ≥95%, m/z (ESI): 424.30 [M+H]⁺. ¹H NMR (500 MHz, DMSO-d₆) δ 13.24 (s, 1H), 9.75 (s, 1H), 8.69 (d, J=6.8 Hz, 1H), 8.42 (d, J=6.7 Hz, 1H), 7.68 (d, J=3.7 Hz, 1H), 7.47 (s, 1H), 6.88 (d, J=3.8 Hz, 1H), 4.34 (s, 3H), 3.19 (s, 3H), 2.18 (s, 3H).

### Synthesis of 5-(2-fluorophenyl)thiophene-2-carboxylic acid

According to the general procedure for Suzuki coupling reactions using Method B, precatalyst C1 and general procedure for saponification. Methyl 5-bromothiophene-2-carboxylate (0.66 g, 2.71 mmol), (2-fluorophenyl)boronic acid (1.5 mmol), toluene/ethanol/water (10:3:2, v/v/v, 15 mL). The crude was taken directly for saponification using the general procedure used for saponification prior to purification. White solid (0.1 g, 17% yield). LCMS (220 nm, 254 nm): t_{R} 2.305 min, purity ≥95%, m/z (ESI): 264.25 [M+CH₃CN]⁺.

### Synthesis of N-(2-bromo-3-methylphenyl)-5-(2-fluorophenyl)-N-methylthiophene-2-carboxamide

According to general procedure C for the amide coupling using T3P. 5-(2-fluorophenyl)thiophene-2-carboxylic acid (86 mg, 0.39 mmol), 2-bromo-N,3-dimethylaniline (92.91 mg, 0.46 mmol), EtOAc (3 mL). Purification using preparative HPLC Method B. Solvent was concentrated under vacuo to give the desired product as a beige solid (32 mg, 21% yield). LCMS (220 nm, 254 nm): t_{R} 2.203 min, purity ≥95%, m/z (ESI): 406.15 [M+H, 100%]⁺.

### Synthesis of 5-{2-methyl-6-[N-methyl-5-(2-fluorophenyl)thiophene-2-amido]phenyl}thiophene-2-carboxylic acid (RU-NT-496)

According to the general procedure for Suzuki coupling reactions using Method A, precatalyst C1 and saponification. N-(2-bromo-3-methylphenyl)-5-(2-fluorophenyl)-N-methylthiophene-2-carboxamide (32 mg, 0.08 mmol), [5-(methoxycarbonyl)thiophen-2-yl]boronic acid (22 mg, 0.12 mmol). The crude was taken directly for the saponification according to the general procedure. Purification using preparative HPLC Method B. Solvent was concentrated under vacuo to give the desired product as a white solid (6 mg, 17% yield). LCMS (220 nm, 254 nm): t_{R} 2.891 min, purity ≥95%, m/z (ESI): 452.25 [M+H, 100%]⁺. ¹H NMR (500 MHz, DMSO-d₆) δ 13.17 (br s, 1H), 7.72 (t, J=7.9 Hz, 1H), 7.69 (d, J=3.7 Hz, 1H), 7.52 (d, J=4.8 Hz, 2H), 7.43 (d, J=4.7 Hz, 1H), 7.39 (t, J=5.9 Hz, 2H), 7.33 (dd, J=11.6, 8.3 Hz, 1H), 7.27 (t, J=7.6 Hz, 1H), 6.88 (d, J=3.8 Hz, 1H), 6.50 (d, J=4.1 Hz, 1H), 3.09 (s, 3H), 2.19 (s, 3H). ¹⁹F NMR (471 MHz, DMSO-d₆) δ-113.18 Hz.

### Synthesis of methyl 5-[2-(4-fluoro-]-benzothiophene-2-amido)-6-methylphenyl]thiophene-2-carboxylate

According to the general procedure for Suzuki coupling reactions using Method A, precatalyst C1. N-(2-bromo-3-methylphenyl)-4-fluoro-1-benzothiophene-2-carboxamide (20 mg, 0.05 mmol), [5-(methoxycarbonyl)thiophen-2-yl]boronic acid (13.28 mg, 0.07 mmol). Purification using preparative HPLC Method B. Solvent was concentrated under vacuo to give the desired product as a white solid (20.7 mg, 89% yield). LCMS (220 nm, 254 nm): t_{R} 3.132 min, purity ≥95%, m/z (ESI): 426.25 [M+H]⁺. ¹H NMR (500 MHz, DMSO-d₆) δ 10.15 (s, 1H), 8.11 (s, 1H), 7.88 (d, J=8.2 Hz, 1H), 7.78 (d, J=3.8 Hz, 1H), 7.50 (td, J=8.0, 5.1 Hz, 1H), 7.44 (t, J=7.7 Hz, 1H), 7.35 (dd, J=7.7, 4.5 Hz, 2H), 7.27 (dd, J=10.5, 7.9 Hz, 1H), 7.13 (d, J=3.8 Hz, 1H), 3.77 (s, 3H), 2.23 (s, 3H).

### Synthesis of methyl 5-[2-methyl-6-(N-methyl-4-fluoro-1-benzothiophene-2-amido)phenyl]thiophene-2-carboxylate

According to the general procedure for N-alkylation of amides (Method B). Methyl 5-[2-(4-fluoro-1-benzothiophene-2-amido)-6-methylphenyl]thiophene-2-carboxylate (20 mg, 0.05 mmol), DMF (1 mL). Purification using preparative HPLC Method B. Solvent was concentrated under vacuo to give the desired product as an off-white solid (20 mg, 97% yield). LCMS (220 nm, 254 nm): t_{R} 3.179 min, purity ≥95%, m/z (ESI): 440.25 [M+H]⁺.

### Synthesis of 5-[2-methyl-6-(N-methyl-4-fluoro-1-benzothiophene-2-amido)phenyl]thiophene-2-carboxylic acid (RU-NT-499)

According to the general procedure for saponification of methyl esters. Methyl 5-[2-methyl-6-(N-methyl-4-fluoro-1-benzothiophene-2-amido)phenyl]thiophene-2-carboxylate (25 mg, 0.06 mmol). Purification using preparative HPLC Method B. Solvent was concentrated under vacuo to give the desired product as a white solid (5.7 mg, 24% yield). LCMS (220 nm, 254 nm): t_{R} 2.759 min, purity ≥95%, m/z (ESI): 426.20 [M+H]⁺. ¹H NMR (500 MHz, DMSO-d₆) δ 13.16 (br s, 1H), 7.77 (d, J=8.2 Hz, 1H), 7.68 (d, J=3.8 Hz, 1H), 7.55 (d, J=4.7 Hz, 2H), 7.51 (q, J=4.2 Hz, 1H), 7.44 (td, J=8.1, 5.1 Hz, 1H), 7.17 (dd, J=10.4, 7.9 Hz, 1H), 6.87-6.79 (m, 2H), 3.14 (s, 3H), 2.19 (s, 3H).

### Synthesis of N-(2-bromo-3-methylphenyl)-7-chloro-1-benzothiophene-2-carboxamide

According to general procedure C for the amide coupling using T3P. 7-Chloro-1-benzothiophene-2-carboxylic acid (300 mg, 1.41 mmol), 2-bromo-3-methylaniline (0.27 mL, 2.12 mmol), EtOAc (3 mL). Purification using preparative HPLC Method B. Solvent was concentrated under vacuo to give the desired product as a white solid (213 mg, 40% yield). LCMS (220 nm, 254 nm): t_{R} 3.393 min, purity ≥95%, m/z (ESI): 382.15 [M+H]⁺. ¹H NMR (500 MHz, DMSO-d₆) δ 10.45 (s, 1H), 8.43 (s, 1H), 8.04 (d, J=8.0 Hz, 1H), 7.65 (d, J=7.7 Hz, 1H), 7.53 (t, J=7.8 Hz, 1H), 7.39 (dd, J=7.6, 2.1 Hz, 1H), 7.37-7.26 (m, 2H), 2.43 (s, 3H).

### Synthesis of methyl 5-[2-(7-chloro-]-benzothiophene-2-amido)-6-methylphenyl]thiophene-2-carboxylate & 5-[2-(7-chloro-]-benzothiophene-2-amido)-6-methylphenyl]thiophene-2-carboxylic acid

According to the general procedure for Suzuki coupling reactions using Method A, precatalyst C2. N-(2-bromo-3-methylphenyl)-7-chloro-1-benzothiophene-2-carboxamide (180 mg, 0.47 mmol), [5-(methoxycarbonyl)thiophen-2-yl]boronic acid (87.94 mg, 0.47 mmol). Temperature: 100° C. Purification using preparative HPLC Method B. Solvent was concentrated under vacuo to give the desired product as a white solid (32.9 mg, 16% yield). LCMS (220 nm, 254 nm): t_{R} 3.371 min, purity ≥95%, m/z (ESI): 442.25 [M+H]⁺. ¹H NMR (500 MHz, DMSO-d₆) δ 10.18 (s, 1H), 8.09 (s, 1H), 7.95 (d, J=8.0 Hz, 1H), 7.77 (d, J=3.8 Hz, 1H), 7.61 (d, J=7.7 Hz, 1H), 7.49 (t, J=7.9 Hz, 1H), 7.43 (d, J=7.7 Hz, 1H), 7.34 (t, J=7.0 Hz, 2H), 7.11 (d, J=3.8 Hz, 1H), 3.77 (s, 3H), 2.22 (s, 3H), and the acid side product as a white solid (73.3 mg, 36% yield). LCMS (220 nm, 254 nm): t_{R} 2.851 min, purity ≥95%, m/z (ESI): 428.20 [M+H]⁺. ¹H NMR (500 MHz, DMSO-d₆) δ 13.11 (br s, 1H), 10.15 (s, 1H), 8.09 (s, 1H), 7.95 (d, J=7.9 Hz, 1H), 7.68 (d, J=3.7 Hz, 1H), 7.60 (d, J=7.7 Hz, 1H), 7.49 (t, J=7.9 Hz, 1H), 7.42 (t, J=7.7 Hz, 1H), 7.38-7.30 (m, 2H), 7.08 (d, J=3.7 Hz, 1H), 2.23 (s, 3H).

### Synthesis of methyl 5-[2-methyl-6-(N-methyl-7-chloro-1-benzothiophene-2-amido)phenyl]thiophene-2-carboxylate

According to the general procedure for N-alkylation of amides (Method B). 5-[2-(7-Chloro-1-benzothiophene-2-amido)-6-methylphenyl]thiophene-2-carboxylic acid (70 mg, 0.16 mmol), DMF (1 mL). Purification using preparative HPLC Method B. Solvent was concentrated under vacuo to give the desired product as a colorless oil (64.2 mg, 86% yield). LCMS (220 nm, 254 nm): t_{R} 3.305 min, purity ≥95%, m/z (ESI): 456.20 [M+H]⁺. ¹H NMR (500 MHz, DMSO-d₆) δ 7.84-7.66 (m, 2H), 7.54 (t, J=5.1 Hz, 2H), 7.50 (d, J=5.3 Hz, 1H), 7.37 (dt, J=27.7, 7.6 Hz, 2H), 6.99 (s, 1H), 6.90 (d, J=3.8 Hz, 1H), 3.79 (s, 3H), 3.13 (s, 3H), 2.19 (s, 3H).

### Synthesis of 5-[2-methyl-6-(N-methyl-7-chloro-1-benzothiophene-2-amido)phenyl]thiophene-2-carboxylic acid (RU-NT-500)

According to the general procedure for saponification of methyl esters. Methyl 5-[2-methyl-6-(N-methyl-7-chloro-1-benzothiophene-2-amido)phenyl]thiophene-2-carboxylate (60 mg, 0.13 mmol). 2-amido)-6-methylphenyl]thiophene-2-carboxylic acid (70 mg, 0.16 mmol), DMF (1 mL). Purification using preparative HPLC Method B. Solvent was concentrated under vacuo to give the desired product as a white solid (11 mg, 19% yield). LCMS (220 nm, 254 nm): t_{R} 2.891 min, purity ≥95%, m/z (ESI): 442.25 [M+H]⁺. ¹H NMR (500 MHz, DMSO-d₆) δ 13.18 (br s, 1H), 7.76 (d, J=8.0 Hz, 1H), 7.68 (d, J=3.8 Hz, 1H), 7.60-7.51 (m, 3H), 7.51-7.47 (m, 1H), 7.40 (t, J=7.9 Hz, 1H), 6.99 (s, 1H), 6.86 (d, J=3.8 Hz, 1H), 3.14 (s, 3H), 2.19 (s, 3H).

### Synthesis of N-(2-bromo-3-methylphenyl)-4-chloro-1-benzothiophene-2-carboxamide

In a 100 mL RBF containing 4-chloro-1-benzothiophene-2-carboxylic acid (300 mg, 1.41 mmol) was added 2-bromo-3-methylaniline (0.27 mL, 2.12 mmol), EtOAc (3 mL). Purification using preparative HPLC Method B. Solvent was concentrated under vacuo to give the desired product as a greyish solid (86.7 mg, 16% yield). LCMS (220 nm, 254 nm): t_{R} 3.367 min, purity ≥95%, m/z (ESI): 381.15 [M+H]⁺.

### Synthesis of methyl 5-[2-(4-chloro-]-benzothiophene-2-amido)-6-methylphenyl]thiophene-2-carboxylate & 5-[2-(4-chloro-]-benzothiophene-2-amido)-6-methylphenyl]thiophene-2-carboxylic acid

According to the general procedure for Suzuki coupling reactions using Method A, precatalyst C1. N-(2-bromo-3-methylphenyl)-4-chloro-1-benzothiophene-2-carboxamide (85 mg, 0.22 mmol), [5-(methoxycarbonyl)thiophen-2-yl]boronic acid (41 mg, 0.22 mmol). Temperature: 115° C. Purification using preparative HPLC Method B. Solvent was concentrated under vacuo to give the desired product as a beige solid (19.1 mg, 19% yield). LCMS (220 nm, 254 nm): t_{R} 3.311 min, purity ≥95%, m/z (ESI): 442.25 [M+H]⁺. ¹H NMR (500 MHz, DMSO-d₆) δ 10.22 (s, 1H), 8.02 (d, J=8.1 Hz, 1H), 7.95 (s, 1H), 7.78 (d, J=3.8 Hz, 1H), 7.54 (d, J=7.7 Hz, 1H), 7.51-7.44 (m, 1H), 7.43 (d, J=7.6 Hz, 1H), 7.35 (d, J=7.7 Hz, 2H), 7.13 (d, J=3.8 Hz, 1H), 3.77 (s, 3H), 2.23 (s, 3H), and the acid side product as a white solid (20 mg, 21% yield). LCMS (220 nm, 254 nm): t_{R} 2.839 min, purity ≥95%, m/z (ESI): 428.10 [M+H]⁺. ¹H NMR (500 MHz, DMSO-d₆) δ 10.22 (s, 1H), 8.02 (d, J=8.1 Hz, 1H), 7.95 (s, 1H), 7.78 (d, J=3.8 Hz, 1H), 7.54 (d, J=7.7 Hz, 1H), 7.51-7.44 (m, 1H), 7.43 (d, J=7.6 Hz, 1H), 7.35 (d, J=7.7 Hz, 2H), 7.13 (d, J=3.8 Hz, 1H), 3.77 (s, 3H), 2.23 (s, 3H).

### Synthesis of methyl 5-[2-methyl-6-(N-methyl-4-chloro-]-benzothiophene-2-amido)phenyl]thiophene-2-carboxylate

According to the general procedure for N-alkylation of amides (Method B). Methyl 5-[2-(4-chloro-1-benzothiophene-2-amido)-6-methylphenyl]thiophene-2-carboxylate (20 mg, 0.05 mmol), DMF (1 mL). White solid (10 mg, 49% yield). LCMS (220 nm, 254 nm): t_{R} 3.336 min, purity ≥95%, m/z (ESI): 478.20 [M+CH₃CN, 100%]⁺.

### Synthesis of 5-[2-methyl-6-(N-methyl-4-chloro-1-benzothiophene-2-amido)phenyl]thiophene-2-carboxylic acid (RU-NT-501)

According to the general procedure for saponification of methyl esters. Methyl 5-[2-methyl-6-(N-methyl-4-chloro-1-benzothiophene-2-amido) phenyl]thiophene-2-carboxylate (10 mg, 0.02 mmol). Purification using preparative HPLC Method B. Solvent was concentrated under vacuo to give the desired product as a white solid (9 mg, 93% yield). LCMS (220 nm, 254 nm): t_{R}2.915 min, purity ≥95%, m/z (ESI): 442.25 [M+H]⁺. ¹H NMR (500 MHz, DMSO-d₆) δ 13.18 (br s, 1H), 7.93 (d, J=7.8 Hz, 1H), 7.68 (d, J=3.8 Hz, 1H), 7.56 (d, J=4.9 Hz, 2H), 7.51 (d, J=5.2 Hz, 1H), 7.48-7.38 (m, 2H), 6.85 (d, J=3.8 Hz, 1H), 6.80 (s, 1H), 3.15 (s, 3H), 2.19 (s, 3H).

### Synthesis of N-(2-bromo-3-methylphenyl)-6-fluoro-1-benzothiophene-2-carboxamide

According to general procedure C for the amide coupling using T3P. 6-fluoro-1-benzothiophene-2-carboxylic acid (250 mg, 1.27 mmol), 2-bromo-3-methylaniline (355.60 mg, 1.91 mmol), EtOAc (3 mL). Purification using preparative HPLC Method B. Solvent was concentrated under vacuo to give the desired product as an off-white solid (200 mg, 43% yield). LCMS (220 nm, 254 nm): t_{R} 3.139 min, purity ≥95%, m/z (ESI): 366.20 [M+H]⁺. ¹H NMR (500 MHz, DMSO-d₆) δ 10.33 (s, 1H), 8.33 (s, 1H), 8.06 (dd, J=8.9, 5.3 Hz, 1H), 7.99 (dd, J=9.3, 2.4 Hz, 1H), 7.45-7.13 (m, 3H), 2.43 (s, 3H). ¹⁹F NMR (471 MHz, DMSO-d₆) δ-113.96 Hz.

### Synthesis of 5-[2-(6-fluoro-1-benzothiophene-2-amido)-6-methylphenyl]thiophene-2-carboxylic acid

According to the general procedure for Suzuki coupling reactions using Method A, precatalyst C1. N-(2-bromo-3-methylphenyl)-6-fluoro-1-benzothiophene-2-carboxamide (180 mg, 0.49 mmol), [5-(methoxycarbonyl)thiophen-2-yl]boronic acid (119.49 mg, 0.64 mmol). Purification using preparative HPLC Method B. Solvent was concentrated under vacuo to give the desired product as an off-white solid (53.8 mg, 27% yield). LCMS (220 nm, 254 nm): t_{R} 2.667 min, purity ≥95%, m/z (ESI): 412.25 [M+H]⁺. ¹H NMR (500 MHz, DMSO-d₆) δ 13.02 (br s, 1H), 10.02 (s, 1H), 8.04-7.91 (m, 3H), 7.69 (d, J=3.7 Hz, 1H), 7.45-7.38 (m, 1H), 7.33 (dd, J=8.6, 2.8 Hz, 3H), 7.08 (d, J=3.7 Hz, 1H), 2.23 (s, 3H). ¹⁹F NMR (471 MHz, DMSO-d₆) δ-114.14.

### Synthesis of methyl 5-[2-methyl-6-(N-methyl-6-fluoro-1-benzothiophene-2-amido)phenyl]thiophene-2-carboxylate

According to the general procedure for N-alkylation of amides (Method B). 5-[2-(6-Fluoro-1-benzothiophene-2-amido)-6-methylphenyl]thiophene-2-carboxylic acid (53 mg, 0.13 mmol), DMF (1 mL). Purification using preparative HPLC Method B. Solvent was concentrated under vacuo to give the desired product as white crystals (24.6 mg, 44% yield). LCMS (220 nm, 254 nm): t_{R} 3.139 min, purity ≥95%, m/z (ESI): 440.25 [M+H]⁺. ¹H NMR (500 MHz, DMSO-d₆) δ 7.87-7.79 (m, 2H), 7.78 (d, J=3.8 Hz, 1H), 7.58-7.50 (m, 2H), 7.46 (d, J=6.2 Hz, 1H), 7.34-7.18 (m, 1H), 7.04 (s, 1H), 6.92 (d, J=3.9 Hz, 1H), 3.79 (s, 3H), 2.20 (s, 3H).

### Synthesis of 5-[2-methyl-6-(N-methyl-6-fluoro-1-benzothiophene-2-amido)phenyl]thiophene-2-carboxylic acid (RU-NT-502)

According to the general procedure for saponification of methyl esters. Methyl 5-[2-methyl-6-(N-methyl-6-fluoro-1-benzothiophene-2-amido)phenyl]thiophene-2-carboxylate (22 mg, 0.05 mmol). Purification using preparative HPLC Method B. Solvent was concentrated under vacuo to give the desired product as a white solid (15 mg, 70% yield). LCMS (220 nm, 254 nm): t_{R} 2.744 min, purity ≥95%, m/z (ESI): 426.20 [M+H]⁺. ¹H NMR (500 MHz, DMSO-d₆) δ 7.82 (ddd, J=14.3, 9.0, 3.9 Hz, 2H), 7.68 (d, J=3.8 Hz, 1H), 7.52 (dd, J=8.6, 5.2 Hz, 2H), 7.45 (dd, J=6.3, 2.9 Hz, 1H), 7.23 (td, J=9.0, 2.5 Hz, 1H), 7.03 (s, 1H), 6.87 (d, J=3.8 Hz, 1H), 3.10 (s, 3H), 2.19 (s, 3H).

### Synthesis of 5-methoxy-3-methyl-1-benzothiophene-2-carboxylic acid

According to the general procedure for the synthesis of substituted benzothiophenes. Methyl 2-mercaptoacetate (1.11 g, 10.48 mmol), 1-(2-bromo-5-methoxyphenyl)ethan-1-one (2.0 g, 8.73 mmol). Purification using preparative HPLC Method B. Solvent was concentrated under vacuo to give the desired product as a white solid (0.11 g, 6% yield) of the ester. Saponification was carried out according to the general procedure and purification of the compound via extraction only with EtOAc and brine 3 times. The combined organic layers were dried over anhydrous MgSO₄ prior to reduction under reduced pressure to give a white solid (0.1 g, 100% yield). LCMS (220 nm, 254 nm): t_{R} 2.233 min, purity ≥95%, m/z (ESI): 443.25 [2M]⁺. ¹H NMR (500 MHz, DMSO-d₆) δ 13.16 (br s, 1H), 7.86 (d, J=8.8 Hz, 1H), 7.38 (d, J=2.5 Hz, 1H), 7.16 (dd, J=8.8, 2.5 Hz, 1H), 3.86 (s, 3H), 2.69 (s, 3H).

### Synthesis of N-(2-bromo-3-methylphenyl)-5-methoxy-N,3-dimethyl-]-benzothiophene-2-carboxamide

According to general procedure C for the amide coupling using T3P. 5-methoxy-3-methyl-1-benzothiophene-2-carboxylic acid (100 mg, 0.45 mmol), 2-bromo-N,3-dimethylaniline (108 mg, 0.54 mmol), DMF (2 mL). Purification using preparative HPLC Method B. Solvent was concentrated under vacuo to give the desired product as a brown oil (10 mg, 6% yield). LCMS (220 nm, 254 nm): t_{R} 3.045 min, purity ≥95%, m/z (ESI): 404.20 [M+H]⁺.

### Synthesis of 5-[2-methyl-6-(N-methyl-5-methoxy-3-methyl-1-benzothiophene-2-amido)phenyl]thiophene-2-carboxylic acid (RU-NT-503)

According to the general procedure for Suzuki coupling reactions using Method A, precatalyst C1. and saponification. N-(2-bromo-3-methylphenyl)-5-methoxy-N,3-dimethyl-1-benzothiophene-2-carboxamide (10 mg, 0.02 mmol), [5-(methoxycarbonyl)thiophen-2-yl]boronic acid (9.2 mg, 0.05 mmol), dry dioxane (1 mL). Temperature: 120° C. Purification using preparative HPLC Method B. Solvent was concentrated under vacuo to give the desired product as a white solid (2.6 mg, 23% yield). LCMS (220 nm, 254 nm): t_{R} 2.736 min, purity ≥95%, m/z (ESI): 452.30 [M+H]⁺. ¹H NMR for the observed rotamers (500 MHz, DMSO-d₆) δ 13.20 (br s, 1H), 7.98-7.56 (m, 2H), 7.54-7.17 (m, 4H), 7.13-6.78 (m, 2H), 3.83 (s, 3H), 3.08 (s, 3H), 2.32 (s, 3H), 2.15 (s, 3H).

### Synthesis of 2-bromo-3,4-dimethyl-1-nitrobenzene

To a solution of 2,3-dimethyl-6-nitroaniline (4.5 g, 27.1 mmol) in MeCN (120 mL) was added tert-butyl nitrite (6.44 mL, 2 eq) drop wise at 0° C. The reaction mixture was stirred at 0° C. for 45 min. Copper(II)bromide (7.26 g, 1.2 eq) was added portion wise to the reaction mixture at 0° C. The reaction mixture was heated at 60° C. overnight. The resulting reaction mixture was combined with 1 other batch prepared on the same scale by an identical method. The reaction mixture was poured into saturated NaHCO₃ solution. The resulting reaction mixture was extracted with EtOAc 3x. The combined organic phase was washed with brine and dried over MgSO₄, filtered and concentrated under reduced pressure. The resulting residue was purified by column chromatography (2.4% EtOAc in hexane) to give the desired product as a yellow solid (3.25 g, 52% yield). t_{R} 3.068 min, purity ≥95%, m/z (ESI): N/A. ¹H NMR (500 MHz, DMSO-d₆) δ 7.67 (d, J=8.2 Hz, 1H), 7.41 (d, J=8.2 Hz, 1H), 2.41 (s, 3H), 2.39 (s, 3H).

### Synthesis of 2-bromo-3,4-dimethylaniline

In a 250 mL RBF, 2-bromo-3,4-dimethyl-1-nitrobenzene (3 g, 13.04 mmol) was dissolved into EtOH (80 mL). To this was added ammonium chloride (535 mg, 10 mmol) dissolved into water (13 mL). This mixture was heated to 80° C. with stirring and then iron (3.64 g, 65.20 mmol) was added. The reaction was then heated to 80° C. with stirring overnight. The reaction mixture was then filtered through Celite®. Extraction was carried out 3x with EtOAc and brine and the combined organic layers were dried over anhydrous MgSO₄. The solvent was concentrated under vacuo. Purification was carried out using Method B, using C18 80 g column. The desired product was obtained as a brown oil (1 g, 38% yield). t_{R} 1.620 min, purity ≥95%, m/z (ESI): 241.15 [M+H]⁺.

### Synthesis of N-(2-bromo-3,4-dimethylphenyl)-3-methyl-1-benzothiophene-2-carboxamide

According to general procedure C for amide coupling. 3-Methyl-1-benzothiophene-2-carboxylic acid (250 mg, 1.3 mmol), 2-bromo-3,4-dimethylaniline (260 mg, 1.3 mmol), EtOAc (3-4 mL). Purification using preparative HPLC Method B. Solvent was concentrated under vacuo to give the desired product as a beige solid (400 mg, 82% yield). LCMS (220 nm, 254 nm): t_{R} 3.543 min, purity ≥95%, m/z (ESI): 376.20 [M+H]⁺.

### Synthesis of methyl 5-[2,3-dimethyl-6-(3-methyl-1-benzothiophene-2-amido) phenyl]thiophene-2-carboxylate

According to the general procedure for Suzuki coupling reactions using Method A, precatalyst C1. N-(2-bromo-3,4-dimethylphenyl)-3-methyl-1-benzothiophene-2-carboxamide (300 mg, 0.8 mmol), [5-(methoxycarbonyl)thiophen-2-yl]boronic acid (298 mg, 1.6 mmol), dry dioxane (5 mL), temperature: 100° C. Purification using preparative HPLC Method B. Solvent was concentrated under vacuo to give the desired product as a white solid (27.7 mg, 8% yield). LCMS (220 nm, 254 nm): t_{R} 3.300 min, purity ≥95%, m/z (ESI): 450.30 [M+H]⁺.

### Synthesis of methyl 5-[2,3-dimethyl-6-(N-methyl-3-methyl-1-benzothiophene-2-amido)phenyl]thiophene-2-carboxylate

According to the general procedure for N-alkylation of amides (Method B). Methyl 5-[2,3-dimethyl-6-(3-methyl-1-benzothiophene-2-amido)phenyl]thiophene-2-carboxylate (27 mg, 0.06 mmol). Purification using preparative HPLC Method B. Solvent was concentrated under vacuo to give the desired product as a clear oil (11 mg, 40% yield). LCMS (220 nm, 254 nm): t_{R} 3.296 min, purity ≥95%, m/z (ESI): 450.30 [M+H]⁺.

### Synthesis of 5-[2,3-dimethyl-6-(N-methyl-3-methyl-]-benzothiophene-2-amido)phenyl]thiophene-2-carboxylic acid (RU-NT-505)

According to the general procedure for saponification of methyl esters. Methyl 5-[2,3-dimethyl-6-(N-methyl-3-methyl-1-benzothiophene-2-amido)phenyl]thiophene-2-carboxylate (10 mg, 0.02 mmol). Purification using preparative HPLC Method B. Solvent was concentrated under vacuo to give the desired product as a white solid (9.3 mg, 96% yield). LCMS (220 nm, 254 nm): t_{R} 2.860 min, purity ≥95%, m/z (ESI): 436.25 [M+H]⁺. ¹H NMR for the observed rotamers (500 MHz, DMSO-d₆) δ 13.18 (s, 1H), 7.97 (s, 1H), 7.87-7.61 (m, 2H), 7.53-7.36 (m, 2H), 7.36-7.16 (m, 1H), 6.82 (d, J=3.8 Hz, 1H), 3.07 (s, 3H), 2.36 (s, 3H), 2.26 (s, 3H), 2.14-1.86 (s, 3H).

### Synthesis of methyl 7-methoxy-1-benzothiophene-2-carboxylate

According to the general procedure for the synthesis of substituted benzothiophenes. 2-Fluoro-3-methoxybenzaldehyde (5 g, 32.44 mmol), methyl 2-sulfanylacetate (4.13 g, 38.93 mmol). Purification in a similar manner to that in the general procedure. The desired product was obtained as an off-white solid (4.9 g, 69% yield). LCMS (220 nm, 254 nm): t_{R} 2.633 min, purity ≥95%, m/z (ESI): N/A. ¹H NMR (500 MHz, DMSO-d₆) δ 8.20 (s, 1H), 7.62 (d, J=8.0 Hz, 1H), 7.45 (t, J=7.9 Hz, 1H), 7.11 (d, J=7.9 Hz, 1H), 3.99 (s, 3H), 3.89 (s, 3H).

### Synthesis of 7-methoxy-1-benzothiophene-2-carboxylic acid

According to the general procedure for saponification of methyl esters. Methyl 7-methoxy-1-benzothiophene-2-carboxylate (1 g, 4.15 mmol). Extraction was carried out 3x with EtOAc and brine and the combined organic layers were dried over anhydrous MgSO₄. The solvent was concentrated under vacuo to give the desired product as a white solid (800 mg, 85% yield). LCMS (220 nm, 254 nm): t_{R} 2.100 min, purity ≥95%, m/z (ESI): 415.20 [2M]⁺.

### Synthesis of N-(2-bromo-3-methylphenyl)-7-methoxy-1-benzothiophene-2-carboxamide

According to general procedure C for amide coupling. 7-Methoxy-1-benzothiophene-2-carboxylic acid (500 mg, 2.4 mmol), 2-bromo-3-methylaniline (580.77 mg, 3.12 mmol). Purification using preparative HPLC Method B. Solvent was concentrated under vacuo to give the desired ester product as a white solid (740 mg, 83% yield). LCMS (220 nm, 254 nm): t_{R} 3.109 min, purity ≥95%, m/z (ESI): 378.15.

### Synthesis of methyl 5-[2-(7-methoxy-1-benzothiophene-2-amido)-6-methylphenyl]thiophene-2-carboxylate

According to the general procedure for Suzuki coupling reactions using Method A, precatalyst C1. N-(2-bromo-3-methylphenyl)-7-methoxy-1-benzothiophene-2-carboxamide (400 mg, 1.06 mmol), [5-(methoxycarbonyl)thiophen-2-yl]boronic acid (217 mg, 1.17 mmol), dry dioxane (10 mL), temperature: 120° C. Purification using preparative HPLC Method B. Solvent was concentrated under vacuo to give the desired ester product as white crystals (110 mg, 24% yield). LCMS (220 nm, 254 nm): t_{R} 3.109 min, purity ≥95%, m/z (ESI): 438.15. ¹H NMR (500 MHz, DMSO-d₆) δ 10.02 (s, 1H), 7.96 (s, 1H), 7.77 (d, J=3.8 Hz, 1H), 7.51 (d, J=8.0 Hz, 1H), 7.41 (dt, J=12.8, 7.8 Hz, 2H), 7.34 (dd, J=7.7, 2.5 Hz, 2H), 7.12 (d, J=3.8 Hz, 1H), 7.03 (d, J=7.8 Hz, 1H), 3.96 (s, 3H), 3.76 (s, 3H), 2.22 (s, 3H).

### Synthesis of methyl 5-[2-methyl-6-(N-methyl-7-methoxy-]-benzothiophene-2-amido)phenyl]thiophene-2-carboxylate

According to the general procedure for N-alkylation of amides (Method B). Methyl 5-[2-(7-methoxy-1-benzothiophene-2-amido)-6-methylphenyl]thiophene-2-carboxylate (110 mg, 0.25 mmol). Purification using preparative HPLC Method B. Solvent was concentrated under vacuo to give the desired ester product as white crystals (90 mg, 79% yield). LCMS (220 nm, 254 nm): t_{R} 3.068 min, purity ≥95%, m/z (ESI): 452.30. ¹H NMR (500 MHz, DMSO-d₆) δ 7.77 (d, J=3.8 Hz, 1H), 7.53 (d, J=5.0 Hz, 2H), 7.45 (s, 1H), 7.32 (d, J=9.1 Hz, 2H), 7.18-6.95 (m, 2H), 6.89 (d, J=3.9 Hz, 1H), 3.90 (s, 3H), 3.79 (s, 3H), 3.10 (s, 3H), 2.19 (s, 3H).

### Synthesis of 3-methyl-5-[2-(trifluoromethyl)phenyl]thiophene-2-carboxylic acid (RU-NT-506)

According to the general procedure for saponification of methyl esters. Methyl 5-[2-methyl-6-(N-methyl-7-methoxy-1-benzothiophene-2-amido)phenyl]thiophene-2-carboxylate (33 mg, 0.07 mmol). Purification using preparative HPLC Method B. Solvent was concentrated under vacuo to give the desired ester product as a white solid (4 mg, 13% yield). LCMS (220 nm, 254 nm): t_{R} 2.687 min, purity ≥95%, m/z (ESI): 438.30. ¹H NMR (500 MHz, DMSO-d₆) δ 13.16 (s, 1H), 7.67 (d, J=3.8 Hz, 1H), 7.55-7.48 (m, 2H), 7.45 (dd, J=6.0, 3.4 Hz, 1H), 7.41-7.26 (m, 2H), 7.05-6.92 (m, 2H), 6.85 (d, J=3.8 Hz, 1H), 3.90 (s, 3H), 3.10 (s, 3H), 2.19 (s, 3H).

### Synthesis of methyl 3-methyl-5-[2-(trifluoromethyl)phenyl]thiophene-2-carboxylate

According to the general procedure for Suzuki coupling reactions using Method B. Methyl 5-bromo-3-methylthiophene-2-carboxylate (0.3 g, 1.28 mmol), [2-(trifluoromethyl)phenyl]boronic acid (0.28 g, 1.91 mmol), toluene/ethanol/water (5:3:2 v/v/v, 7 mL), temperature: 120° C. Purification using preparative HPLC Method B. Solvent was concentrated under vacuo to give the desired ester product as a white solid (0.17 g, 44% yield). LCMS (220 nm, 254 nm): t_{R} 3.118 min, purity ≥95%, m/z (ESI): 342.30 [M+CH₃CN]⁺.

### Synthesis of 3-methyl-5-[2-(trifluoromethyl)phenyl]thiophene-2-carboxylic acid

According to the general procedure for saponification of methyl esters. methyl 3-Methyl-5-[2-(trifluoromethyl)phenyl]thiophene-2-carboxylate (165 mg, 0.55 mmol). Purification using preparative HPLC Method B. Solvent was concentrated under vacuo to give the desired ester product as a white solid (140 mg, 89% yield). LCMS (220 nm, 254 nm): t_{R} 2.667 min, purity ≥95%, m/z (ESI): N/A. ¹⁹F NMR (471 MHz, DMSO-d₆) δ-56.46 Hz.

### Synthesis of N-(2-bromo-3-methylphenyl)-N,3-dimethyl-5-[2-(trifluoromethyl)phenyl]thiophene-2-carboxamide

According to general procedure C for amide coupling. 3-Methyl-5-[2-(trifluoromethyl)phenyl]thiophene-2-carboxylic acid (140 mg, 0.49 mmol), 2-bromo-N,3-dimethylaniline (107.63 mg, 0.54 mmol). Purification using preparative HPLC Method B. Solvent was concentrated under vacuo to give the desired ester product as a white solid (19 mg, 8% yield). LCMS (220 nm, 254 nm): t_{R} 3.428 min, purity ≥95%, m/z (ESI): 469.55 [M+H]⁺.

### Synthesis of 5-[2-methyl-6-(N-methyl-7-fluoro-3-methyl-]-benzothiophene-2-amido)phenyl]thiophene-2-carboxylic acid (RU-NT-507)

According to the general procedure for Suzuki coupling reactions using Method A, precatalyst C1 and general procedure for saponification of methyl esters. N-(2-Bromo-3-methylphenyl)-N,3-dimethyl-5-[2-(trifluoromethyl)phenyl]thiophene-2-carboxamide (19 mg, 0.04 mmol), [5-(methoxycarbonyl)thiophen-2-yl]boronic acid (9.81 mg, 0.05 mmol), dry dioxane (1 mL), temperature: 120° C. Purification using preparative HPLC Method B. Solvent was concentrated under vacuo to give the desired ester product as a white solid (5 mg, 26% yield). LCMS (220 nm, 254 nm): t_{R} 3.077 min, purity ≥95%, m/z (ESI): 516.20 [M+H]⁺. ¹H NMR (500 MHz, DMSO-d₆) δ 13.15 (s, 1H), 7.80 (d, J=7.8 Hz, 1H), 7.74-7.48 (m, 3H), 7.36 (d, J=5.6 Hz, 4H), 6.83 (s, 1H), 6.70 (s, 1H), 3.15 (s, 3H), 2.15 (s, 3H), 2.12 (s, 3H).

### Synthesis of N-(2-bromo-3-methylphenyl)-7-fluoro-3-methyl-1-benzothiophene-2-carboxamide

According to general procedure C for amide coupling. 7-Fluoro-3-methyl-1-benzothiophene-2-carboxylic acid (500 mg, 2.38 mmol), 2-bromo-3-methylaniline (531 mg, 2.85 mmol). Extraction was carried out 3x with EtOAc and brine and the combined organic layers were dried over anhydrous MgSO₄. The solvent was concentrated under vacuo and the sample was fractionated and purification where necessary was carried out using preparative HPLC Method B. Solvent was concentrated under vacuo to give the desired ester product as a white solid (620 mg, 69% yield). LCMS (220 nm, 254 nm): t_{R} 3.473 min, purity ≥95%, m/z (ESI): 419.15 [M+CH₃CN]⁺.

### Synthesis of 5-[2-(7-fluoro-3-methyl-]-benzothiophene-2-amido)-6-methylphenyl]thiophene-2-carboxylic acid & methyl 5-[2-(7-fluoro-3-methyl-1-benzothiophene-2-amido)-6-methylphenyl]thiophene-2-carboxylate

According to the general procedure for Suzuki coupling reactions using Method A, precatalyst C1. N-(2-bromo-3-methylphenyl)-7-fluoro-3-methyl-1-benzothiophene-2-carboxamide (120 mg, 0.32 mmol), [5-(methoxycarbonyl)thiophen-2-yl]boronic acid (76.71 mg, 0.41 mmol), dry dioxane (4 mL), temperature: 120° C. Purification using preparative HPLC Method B. Solvent was concentrated under vacuo to give the desired ester product as a white solid (29.4 mg, 21% yield). t_{R} 3.352 min, purity ≥95%, m/z (ESI): 440.20 [M+H]⁺ and the acid side product as a white solid (39.6 mg, 28% yield). LCMS (220 nm, 254 nm): t_{R} 2.897 min, purity ≥95%, m/z (ESI): 426.25 [M+H]⁺

### Synthesis of methyl 5-[2-methyl-6-(N-methyl-7-fluoro-3-methyl-1-benzothiophene-2-amido)phenyl]thiophene-2-carboxylate

According to the general procedure for N-alkylation of amides (Method B). 5-[2-(7-Fluoro-3-methyl-1-benzothiophene-2-amido)-6-methylphenyl]thiophene-2-carboxylic acid (37 mg, 0.09 mmol). Purification using preparative HPLC Method B. Solvent was concentrated under vacuo to give the desired product as a white solid (15.6 mg, 40% yield). LCMS (220 nm, 254 nm): t_{R} 3.236 min, purity ≥95%, m/z (ESI): 473.20 [M+Na]⁺.

### Synthesis of 5-[2-methyl-6-(N-methyl-7-fluoro-3-methyl-]-benzothiophene-2-amido)phenyl]thiophene-2-carboxylic acid (RU-NT-508)

According to the general procedure for saponification of methyl esters. Methyl 5-[2-methyl-6-(N-methyl-7-fluoro-3-methyl-1-benzothiophene-2-amido)phenyl]thiophene-2-carboxylate (15 mg, 0.03 mmol). Purification using preparative HPLC Method B. Solvent was concentrated under vacuo to give the desired product as a white solid (10 mg, 71% yield). LCMS (220 nm, 254 nm): t_{R} 2.807 min, purity ≥95%, m/z (ESI): 440.20 [M+H]⁺. ¹H NMR for the observed rotamers (500 MHz, DMSO-d₆) δ 13.15 (s, 1H), 7.90-7.54 (m, 2H), 7.56-6.99 (m, 5H), 6.83 (s, 1H), 3.11 (d, J=48.0 Hz, 3H), 2.32 (s, 3H), 2.15 (d, J=34.3 Hz, 3H).

### Synthesis of 5-[2-methyl-6-(N-methyl-7-hydroxy-]-benzothiophene-2-amido)phenyl]thiophene-2-carboxylic acid (RU-NT-509)

To a mixture of 5-[2-methyl-6-(N-methyl-7-methoxy-1-benzothiophene-2-amido)phenyl]thiophene-2-carboxylic acid (24 mg, 0.13 mmol) in dry DCM (2.5 mL) in a 20 mL scintillation vial was added a solution of boron tribromide (1.5 eq) at room temperature and stirred under N₂ overnight. The reaction was quenched with methanol and purification using preparative HPLC Method B. Solvent was concentrated under vacuo to give the desired product as a white solid. LCMS (220 nm, 254 nm): t_{R} 2.357 min, purity ≥95%, m/z (ESI): 424.20 [M+H]⁺. ¹H NMR (500 MHz, DMSO-d₆) δ 13.13 (br s, 1H), 10.39 (s, 1H), 7.68 (d, J=3.7 Hz, 1H), 7.51 (d, J=5.3 Hz, 1H), 7.47-7.38 (m, 1H), 7.17 (d, J=10.5 Hz, 2H), 6.97 (s, 1H), 6.86 (s, 1H), 6.79 (d, J=7.2 Hz, 1H), 6.52 (s, 1H), 3.09 (s, 3H), 2.19 (s, 3H).

### Synthesis of N-bromo-N-(2,3-dimethylphenyl)thieno[2,3-b]pyrazine-6-carboxamide

According to general procedure C for amide coupling. 2-Bromo-N,3-dimethylaniline (122.15 mg, 0.61 mmol), thieno[2,3-b]pyrazine-6-carboxylic acid (100 mg, 0.56 mmol), EtOAc (3 mL). Purification using preparative HPLC Method B. Solvent was concentrated under vacuo to give the desired product as a white solid (100 mg, 50% yield). LCMS (220 nm, 254 nm): t_{R} 2.487 min, purity ≥95%, m/z (ESI): 403.15 [M+CH₃CN]⁺.

### Synthesis of 5-(2-methyl-6-{N-methylthieno[2,3-b]pyrazine-6-amido}phenyl)thiophene-2-carboxylic acid (RU-NT-511)

According to the general procedure for Suzuki coupling reactions using Method A, precatalyst C1. N-(2-bromo-3-methylphenyl)-N-methylthieno[2,3-b]pyrazine-6-carboxamide (100 mg, 0.28 mmol), [5-(methoxycarbonyl)thiophen-2-yl]boronic acid (66.75 mg, 0.36 mmol), dry dioxane (1.5 mL), temperature: 120° C. Purification using preparative HPLC Method B. Solvent was concentrated under vacuo to give the desired product as a the mono TFA salt (3 mg, 3% yield). LCMS (220 nm, 254 nm): t_{R} 2.247 min, purity ≥95%, m/z (ESI): 410.25 [M+H]⁺. ¹H NMR (500 MHz, DMSO-d₆) δ 13.21 (br s, 1H), 8.74 (s, 1H), 8.67 (s, 1H), 7.68 (d, J=3.8 Hz, 1H), 7.57 (s, 2H), 6.87 (d, J=3.9 Hz, 1H), 6.75 (s, 1H), 3.19 (s, 3H), 2.19 (s, 3H).

### Synthesis of methyl 7-fluoro-3-methyl-I-benzothiophene-2-carboxylate

According to the general procedure for the synthesis of substituted benzothiophenes. 1-(2,3-Difluorophenyl)ethan-1-one (5 g, 32.02 mmol), methyl 2-sulfanylacetate (5.10 g, 48.04 mmol). The sample was then purified using the C18 80 column using Method B purification solvents to give the desired product as white crystals (2.3 g, 32% yield). LCMS (220 nm, 254 nm): t_{R} 2.936 min, purity ≥95%, m/z (ESI): 225.30 [M+H]⁺. ¹H NMR (500 MHz, DMSO-d₆) δ 7.87 (d, J=8.0 Hz, 1H), 7.55 (td, J=8.0, 5.2 Hz, 1H), 7.45 (dd, J=10.1, 7.9 Hz, 1H), 3.89 (s, 3H), 2.75 (s, 3H). ¹⁹F NMR (471 MHz, DMSO-d₆) δ-115.93 Hz.

### Synthesis of 7-fluoro-3-methyl-I-benzothiophene-2-carboxylic acid

According to the general procedure for saponification of methyl esters. Methyl 7-fluoro-3-methyl-1-benzothiophene-2-carboxylate (2 g, 8.92 mmol), THF (10 mL), MeOH (10 mL), water (5 mL). Acidification was carried out with 2M HCl after overnight stirring at RT in a 50 mL RBF and extraction was carried out 3x with EtOAc and brine and the combined organic layers were dried over anhydrous MgSO₄. The solvent was concentrated under vacuo and the desired was obtained as a white solid (1.8 g, 100% yield). LCMS (220 nm, 254 nm): t_{R} 2.340 min, purity ≥95%, m/z (ESI): 273.35 [M+CH₃CN+Na]⁺. ¹H NMR (500 MHz, DMSO-d₆) δ 12.5736 (br s, 1H), 7.81 (d, J=8.1 Hz, 1H), 7.51 (td, J=8.0, 5.2 Hz, 1H), 7.40 (dd, J=10.1, 7.8 Hz, 1H), 2.71 (s, 3H). ¹⁹F NMR (471 MHz, DMSO-d₆) δ-115.95 Hz.

### Synthesis of N-(2-bromo-3,4-dimethylphenyl)-7-fluoro-3-methyl-1-benzothiophene-2-carboxamide

According to general procedure C for amide coupling. 7-Fluoro-3-methyl-1-benzothiophene-2-carboxylic acid (1 eq) was added 2-bromo-3,4-dimethylaniline (305.93 mg, 1.53 mmol), EtOAc (4 mL). Purification using preparative HPLC Method B. Solvent was concentrated under vacuo to give the desired product as a brownish oil not 100% pure (assume quantitative yield). LCMS (220 nm, 254 nm): t_{R} 3.576 min, purity ≥95%, m/z (ESI): 393.30 [M+H]⁺.

### Synthesis of methyl 5-[6-(7-fluoro-3-methyl-1-benzothiophene-2-amido)-2,3-dimethylphenyl]thiophene-2-carboxylate

According to the general procedure for Suzuki coupling reactions using Method A, precatalyst C1. N-(2-bromo-3,4-dimethylphenyl)-7-fluoro-3-methyl-1-benzothiophene-2-carboxamide (200 mg, 0.51 mmol), [5-(methoxycarbonyl)thiophen-2-yl]boronic acid (189 mg, 1.02 mmol), dry dioxane (3 mL), temperature: 120° C. Purification using preparative HPLC Method B. Solvent was concentrated under vacuo to give the desired product as a white solid (25.9 mg, 11% yield). LCMS (220 nm, 254 nm): t_{R} 3.443 min, purity ≥95%, m/z (ESI): 454.30 [M+H]⁺.

### Synthesis of methyl 5-[2,3-dimethyl-6-(N-methyl-7-fluoro-3-methyl-]-benzothiophene-2-amido)phenyl]thiophene-2-carboxylate

According to the general procedure for N-alkylation of amides (Method B). Methyl 5-[6-(7-fluoro-3-methyl-1-benzothiophene-2-amido)-2,3-dimethylphenyl]thiophene-2-carboxylate (20 mg, 0.04 mmol). Purification using preparative HPLC Method B. Solvent was concentrated under vacuo to give the desired product as a beige solid (20.6 mg, 30% yield). LCMS (220 nm, 254 nm): t_{R} 3.357 min, purity ≥95%, m/z (ESI): 468.25 [M+H]⁺.

### Synthesis of 5-[2,3-dimethyl-6-(N-methyl-7-fluoro-3-methyl-]-benzothiophene-2-amido)phenyl]thiophene-2-carboxylic acid (RU-NT-512)

According to the general procedure for Suzuki coupling reactions using Method A, precatalyst C1. N-(2-Bromo-3-methylphenyl)-2,3-dimethylbenzamide (300 mg, 1.01 mmol), [5-(methoxycarbonyl)thiophen-2-yl]boronic acid (207.21 mg, 1.11 mmol), dry dioxane (13 mL), temperature: 100° C. Purification using preparative HPLC Method B. Solvent was concentrated under vacuo to give the desired product as a white solid (5 mg, 26% yield). LCMS (220 nm, 254 nm): t_{R} 2.924 min, purity ≥95%, m/z (ESI): 454.15 [M+H]⁺. ¹H NMR for the observed rotamers (500 MHz, DMSO-d₆) δ 12.98 (br s, 1H), 7.98-7.45 (m, 2H), 7.43 (d, J=20.5 Hz, 1H), 7.29 (t, J=8.1 Hz, 2H), 7.06 (d, J=14.3 Hz, 1H), 6.83-6.62 (m, 1H), 3.08 (s, 3H), 2.27 (s, 3H), 2.07 (s, 3H), 1.98 (s, 3H).

### Synthesis of 5-[(2-bromo-3-methylphenyl)(methyl)carbamoyl]thiophene-2-carboxylic acid

According to the general procedure for saponification of methyl esters. Methyl 5-[(2-bromo-3-methylphenyl)(methyl)carbamoyl]thiophene-2-carboxylate (2.8 g, 7.6 mmol), THF (10 mL), MeOH (10 mL), water (5 mL). Acidification was carried out with 2M HCl after overnight stirring at RT in a 50 mL RBF and extraction was carried out 3x with EtOAc and brine. The combined organic layers were dried over anhydrous MgSO₄ and solvent was concentrated under reduced pressure. The desired product was obtained as a beige solid (2.5 g, 93% yield). LCMS (220 nm, 254 nm): t_{R} 2.270 min, purity ≥95%, m/z (ESI): 356.05 [M+H]⁺.

### Synthesis of N-2-(2-bromo-3-methylphenyl)-N-5-methanesulfonyl-N-2-methylthiophene-2,5-dicarboxamide

According to general procedure D for amide coupling. To a solution of 5-[(2-bromo-3-methylphenyl)(methyl)carbamoyl]thiophene-2-carboxylic acid (100 mg, 0.28 mmol) in CH₂CI₂ (1 mL) was added EDCI (2 eq), DMAP (2 eq) and methanesulfonamide (29.54 mg, 1.1 eq). After stirring for 12 h at rt, the solution was diluted with CH₂CI₂ (5 mL) and washed with 2 M HCl until a pH of 2 and brine. Extraction was carried out three times. The organic layers were combined and dried over anhydrous MgSO₄ and the solvent was removed under reduced pressure. Purification using preparative HPLC Method B. Solvent was concentrated under vacuo to give the desired product as a white solid (30 mg, 25% yield). LCMS (220 nm, 254 nm): t_{R} 2.183 min, purity ≥95%, m/z (ESI): 472.15 [M+CH₃CN]⁺.

### Synthesis of 5-{2-methyl-6-[N-methyl-5-(methanesulfonylcarbamoyl)thiophene-2-amido]phenyl}thiophene-2-carboxylic acid (RU-NT-514)

According to the general procedure for Suzuki coupling reactions using Method B, precatalyst C1. N-2-(2-bromo-3-methylphenyl)-N-5-methanesulfonyl-N-2-methylthiophene-2,5-dicarboxamide (20 mg, 0.05 mmol), [5-(methoxycarbonyl)thiophen-2-yl]boronic acid (25.87 mg, 0.14 mmol), dry dioxane (1 mL), temperature: 120° C. Purification using preparative HPLC Method B. Solvent was concentrated under vacuo to give the desired product as a white solid (1 mg, 5% yield). LCMS (220 nm, 254 nm): t_{R} 2.120 min, purity ≥95%, m/z (ESI): 501.15 [M+Na]⁺. ¹H NMR (500 MHz, MeOD-d₄) δ 7.70 (d, J=3.8 Hz, 1H), 7.57 (d, J=4.2 Hz, 1H), 7.53 (d, J=5.9 Hz, 2H), 7.40 (dd, J=6.1, 3.0 Hz, 1H), 6.82 (d, J=4.2 Hz, 1H), 3.30 (s, 3H), 3.24 (s, 3H), 2.24 (s, 3H).

### Synthesis of N-(2-bromo-3-methylphenyl)naphthalene-2-carboxamide

According to general procedure C for amide coupling. Naphthalene-2-carboxylic acid (400 mg, 2.32 mmol), 2-bromo-3-methylaniline (0.34 mL, 2.56 mmol), EtOAc (3 mL). Extraction was carried out according to the general procedure using EtOAc. The desired product was obtained as a beige solid (790 mg, 100% yield). LCMS (220 nm, 254 nm): t_{R} 3.075 min, purity ≥95%, m/z (ESI): 341.20 [M+H]⁺.

### Synthesis of methyl 5-[2-methyl-6-(naphthalene-2-amido)phenyl]thiophene-2-carboxylate

According to the general procedure for Suzuki coupling reactions using Method B, precatalyst C1. N-(2-bromo-3-methylphenyl)naphthalene-2-carboxamide (250 mg, 0.73 mmol), [5-(methoxycarbonyl)thiophen-2-yl]boronic acid (150 mg, 0.81 mmol), dry dioxane (4.9 mL), temperature: 100° C. Purification using preparative HPLC Method B. Solvent was concentrated under vacuo to give the desired product as a white solid (100 mg, 34% yield). LCMS (220 nm, 254 nm): t_{R} 3.017 min, purity ≥95%, m/z (ESI): 402.20 [M+H]⁺.

### Synthesis of methyl 5-[2-methyl-6-(N-methylnaphthalene-2-amido)phenyl]thiophene-2-carboxylate

According to the general procedure for N-alkylation of amides (Method B). Methyl 5-[2-methyl-6-(naphthalene-2-amido)phenyl]thiophene-2-carboxylate (100 mg, 0.25 mmol). Extraction was carried out according to the general procedure and the crude taken directly to the saponification without any further purification. LCMS (220 nm, 254 nm): t_{R} 3.013 min, purity=82%, m/z (ESI): 416.20 [M+H]⁺.

### Synthesis of 5-[2-methyl-6-(N-methylnaphthalene-2-amido)phenyl]thiophene-2-carboxylic acid (RU-NT-515)

According to the general procedure for saponification of methyl esters. 5-[2-Methyl-6-(N-methylnaphthalene-2-amido)phenyl]thiophene-2-carboxylic acid, crude from the previous step. Purification using preparative HPLC Method B. Solvent was concentrated under vacuo to give the desired product as a white solid (21.2 mg, 21% yield). LCMS (220 nm, 254 nm): t_{R} 2.610 min, purity ≥95%, m/z (ESI): 402.25 [M+H]⁺. ¹H NMR for the observed rotamers (500 MHz, DMSO-d₆) δ 13.22 (s, 1H), 8.24-7.78 (m, 2H), 7.63-7.39 (m, 3H), 7.29-7.01 (m, 3H), 6.68 (d, J=3.8 Hz, 1H), 3.20 (s, 3H), 2.07 (s, 3H).

### Synthesis of methyl 5-bromo-3-methyl-1-benzothiophene-2-carboxylate

According to the general procedure for the synthesis of substituted benzothiophenes. 1-(5-Bromo-2-fluorophenyl)ethan-1-one (10 g, 46 mmol), methyl 2-sulfanylacetate (5.87 g, 1.2 eq). Purification was carried out according to the general procedure. The desired product was obtained as beige crystals (5.9 g, 45% yield). LCMS (220 nm, 254 nm): t_{R} 3.187 min, purity ≥95%, m/z (ESI): N/A. ¹H NMR (500 MHz, DMSO-d₆) δ 8.21 (d, J=1.9 Hz, 1H), 8.01 (d, J=8.6 Hz, 1H), 7.70 (dd, J=8.6, 1.9 Hz, 1H), 3.88 (s, 3H), 2.71 (s, 3H).

### Synthesis of methyl 5-ethenyl-3-methyl-I-benzothiophene-2-carboxylate

Into a 250-mL round-bottom flask purged and maintained with an inert atmosphere of nitrogen, was placed methyl 5-bromo-3-methyl-1-benzothiophene-2-carboxylate (2 g, 7.01 mmol, 1.00 eq), 2-ethenyl-4,4,5,5-tetramethyl-1,3,2-dioxaborolane (1.62 g, 10.52 mmol, 1.50 eq), Pd(dppf)Cl₂.DCM (0.20 eq), potassium carbonate (2.91 g 3.00 eq), water (6 mL), dioxane (60 mL). The resulting solution was stirred overnight at 100° C. The mixture was cooled to 20° C. Extraction was carried out 3x with EtOAc and brine, the organic layer was combined and dried over anhydrous MgSO₄. The desired product was taken directly to the next step without further purification. An aliquot of the crude was confirmed by ¹H-NMR for the presence of the olefin group.

### Synthesis of 5-ethenyl-3-methyl-1-benzothiophene-2-carboxylic acid

According to the general procedure for saponification of methyl esters. methyl 5-Ethenyl-3-methyl-1-benzothiophene-2-carboxylate (2.79 g, 12.01 mmol), MeOH (20 mL), THF (20 mL), water (10 mL). Acidification with 2M HCl until a pH of 2-3 and extraction was carried out 3x with EtOAc and brine, the organic layer was combined and dried over anhydrous MgSO₄. The desired product was obtained as a beige solid (2 g, 76% yield). LCMS (220 nm, 254 nm): t_{R} 2.516 min, m/z (ESI): 282.60 [M+CH₃CN+Na]⁺.

### Synthesis of N-(2-bromo-3-methylphenyl)-5-ethenyl-3-methyl-1-benzothiophene-2-carboxamide

According to general procedure C for amide coupling. 5-Ethenyl-3-methyl-1-benzothiophene-2-carboxylic acid (2 g, 9.16 mmol), 2-bromo-3-methylaniline (2.22 g, 11.91 mmol), DMF (6 mL). Purification using preparative HPLC Method B using C18 80 g size column. Solvent was concentrated under vacuo to give the desired product as a beige solid (1 g, 28% yield). LCMS (220 nm, 254 nm): t_{R} 3.617 min, purity=94%, m/z (ESI): 387.15 [M+H]⁺. H NMR (500 MHz, DMSO-d₆) δ 9.86 (s, 1H), 8.20-7.79 (m, 1H), 7.71 (dd, J=8.4, 1.7 Hz, 1H), 7.65-7.41 (m, 3H), 7.39-7.21 (m, 1H), 6.92 (dd, J=17.7, 11.0 Hz, 1H), 5.99 (dd, J=17.6, 0.9 Hz, 1H), 5.61-5.12 (m, 1H), 2.74 (s, 3H), 2.43 (s, 3H).

### Synthesis of methyl 5-[2-(5-ethenyl-3-methyl-]-benzothiophene-2-amido)-6-methylphenyl]thiophene-2-carboxylate

According to the general procedure for Suzuki coupling reactions using Method B, precatalyst C1. N-(2-bromo-3-methylidenephenyl)-5-ethenyl-3-methyl-1-benzothiophene-2-carboxamide (0.97 g, 2.52 mmol). [5-(Methoxycarbonyl)thiophen-2-yl]boronic acid (0.7 g, 3.78 mmol), dry dioxane (16 mL), temperature: 100° C. Purification using preparative HPLC Method B. Solvent was concentrated under vacuo to give the desired product as a brownish solid (520 mg, 46% yield). LCMS (220 nm, 254 nm): t_{R} 3.457 min, purity ≥95%, m/z (ESI): 448.20 [M+H]⁺. ¹H NMR (500 MHz, DMSO-d₆) δ 9.50 (s, 1H), 7.95 (d, J=8.4 Hz, 1H), 7.90 (d, J=1.6 Hz, 1H), 7.85 (d, J=3.8 Hz, 1H), 7.65 (dd, J=8.5, 1.7 Hz, 1H), 7.48 (d, J=7.8 Hz, 1H), 7.44 (t, J=7.7 Hz, 1H), 7.32 (d, J=7.3 Hz, 1H), 7.14 (d, J=3.8 Hz, 1H), 6.88 (dd, J=17.6, 11.0 Hz, 1H), 5.97 (dd, J=17.7, 0.9 Hz, 1H), 5.32 (d, J=11.1 Hz, 1H), 3.82 (s, 3H), 2.36 (s, 3H), 2.20 (s, 3H).

### Synthesis of methyl 5-[2-methyl-6-(N-methyl-5-ethenyl-3-methyl-]-benzothiophene-2-amido)phenyl]thiophene-2-carboxylate

According to the general procedure for N-alkylation of amides (Method B). Methyl 5-[2-(5-ethenyl-3-methyl-1-benzothiophene-2-amido)-6-methylphenyl]thiophene-2-carboxylate (520 mg, 1.16 mmol). The sample was fractionated and purification using preparative HPLC Method B. Solvent was concentrated under vacuo to give the desired product as a brownish solid (0.5 g, 93% yield). LCMS (220 nm, 254 nm): t_{R} 3.320 min, m/z (ESI): 507.20 [M+CH₃CN+Na]⁺.

### Synthesis of 5-[2-methyl-6-(N-methyl-5-ethenyl-3-methyl-]-benzothiophene-2-amido)phenyl]thiophene-2-carboxylic acid (RU-NT-516)

According to the general procedure for saponification of methyl esters. Methyl 5-[2-methyl-6-(N-methyl-5-ethenyl-3-methyl-1-benzothiophene-2-amido)phenyl]thiophene-2-carboxylate (12 mg, 0.03 mmol). Purification using preparative HPLC Method B. Solvent was concentrated under vacuo to give the desired product as a white solid (4 mg, 34% yield). LCMS (220 nm, 254 nm): t_{R} 2.917 min, purity ≥95%, m/z (ESI): 448.25 [M+H]⁺. ¹H NMR for the observed rotamers (500 MHz, DMSO-d₆) δ 13.04 (br s, 1H), 8.10-7.68 (m, 3H), 7.61-7.45 (m, 1H), 7.49-7.18 (m, 2H), 7.16-6.52 (m, 3H), 5.94 (t, J=16.9 Hz, 1H), 5.30 (d, J=11.1 Hz, 1H), 3.13 (s, 3H), 2.34 (s, 3H), 2.10 (s, 3H).

### Synthesis of N-(2-bromo-3-methylphenyl)-2,3-dimethylbenzamide

According to general procedure C for amide coupling. 2-Bromo-3-methylaniline (545 mg, 2.93 mmol), 2,3-dimethylbenzoic acid (400 mg, 0.26 mmol), EtOAc (3 mL). Purification using preparative HPLC Method B. Solvent was concentrated under vacuo to give the desired product as a white solid (400 mg, 47% yield). LCMS (220 nm, 254 nm): t_{R} 2.897 min, purity ≥95%, m/z (ESI): 319.15 [M+H]⁺. ¹H NMR (500 MHz, DMSO-d₆) δ 9.86 (s, 1H), 7.41 (d, J=7.7 Hz, 1H), 7.36 (d, J=7.4 Hz, 1H), 7.31 (t, J=7.7 Hz, 1H), 7.29-7.26 (m, 2H), 7.20 (t, J=7.5 Hz, 1H), 2.41 (s, 3H), 2.33 (s, 3H), 2.29 (s, 3H).

### Synthesis of methyl 5-[2-methyl-6-(N-methyl-2,3-dimethylbenzamido)phenyl]thiophene-2-carboxylate

According to the general procedure for N-alkylation of amides (Method B). Methyl 5-[2-(2,3-dimethylbenzamido)-6-methylphenyl]thiophene-2-carboxylate (155 mg, 0.4 mmol). Extraction was carried out according to the general procedure with DCM and the crude yellow oil was taken directly for the saponification. t_{R} 2.910 min, m/z (ESI): 394.20 [M+H]⁺. Purification using preparative HPLC Method B. Solvent was concentrated under vacuo to give the desired product as a white solid (160 mg, 42% yield). LCMS (220 nm, 254 nm): t_{R} 2.860 min, purity ≥95%, m/z (ESI): 380.15 [M+H]⁺. ¹H NMR (500 MHz, DMSO-d₆) δ 9.59 (s, 1H), 7.83 (d, J=3.7 Hz, 1H), 7.41 (t, J=7.7 Hz, 1H), 7.35 (d, J=7.8 Hz, 1H), 7.29 (d, J=7.5 Hz, 1H), 7.19 (d, J=7.5 Hz, 1H), 7.09 (d, J=3.7 Hz, 1H), 7.06 (d, J=7.6 Hz, 1H), 6.93 (d, J=7.6 Hz, 1H), 3.83 (s, 3H), 2.22 (s, 3H), 2.19 (s, 3H), 2.05 (s, 3H).

### Synthesis of 5-[2-methyl-6-(N-methyl-2,3-dimethylbenzamido)phenyl]thiophene-2-carboxylic acid (RU-NT-517)

According to the general procedure for saponification of methyl esters. Crude from the previous step. Purification using preparative HPLC Method B. Solvent was concentrated under vacuo to give the desired product as a white solid (90 mg, 59% yield). LCMS (220 nm, 254 nm): t_{R} 2.473 min, purity ≥95%, m/z (ESI): 380.20 [M+H]⁺. ¹H NMR for the observed rotamers (500 MHz, DMSO-d₆) δ 7.77 (d, J=3.7 Hz, 1H), 7.49 (d, J=7.8 Hz, 1H), 7.43-7.15 (m, 2H), 7.11 (d, J=3.7 Hz, 3H), 7.05-6.57 (m, 1H), 2.81 (s, 3H), 2.30-2.11 (m, 6H), 2.08 (s, 3H).

### Synthesis of methyl 5-(3-fluoro-2-methoxyphenyl)-3-methylthiophene-2-carboxylate

According to the general procedure for Suzuki coupling reactions using Method B, precatalyst C1. Methyl 5-bromo-3-methylthiophene-2-carboxylate (0.4 g, 1.7 mmol), (3-fluoro-2-methoxyphenyl)boronic acid (0.43 g, 2.55 mmol), toluene (5 mL), EtOH (3 mL), water (1 mL). Purification using preparative HPLC Method B. Solvent was concentrated under vacuo to give the desired product as a white solid (180 mg, 38% yield). LCMS (220 nm, 254 nm): t_{R} 3.090 min, purity ≥95%, m/z (ESI): 281.25 [M+H]⁺. ¹H NMR (500 MHz, DMSO-d₆) δ 7.69 (d, J=8.0 Hz, 1H), 7.63 (s, 1H), 7.48-7.26 (m, 1H), 7.20 (td, J=8.1, 5.3 Hz, 1H), 3.91 (d, J=1.6 Hz, 3H), 3.81 (s, 3H), 2.49 (s, 3H). ¹⁹F NMR (471 MHz, DMSO-d₆) δ-129.81 Hz.

### Synthesis of 5-(3-fluoro-2-methoxyphenyl)-3-methylthiophene-2-carboxylic acid

According to the general procedure for saponification of methyl esters. methyl 5-(3-Fluoro-2-methoxyphenyl)-3-methylthiophene-2-carboxylate (140 mg, 0.5 mmol). Purification using preparative HPLC Method B. Solvent was concentrated under vacuo to give the desired product as a white solid (130 mg, 98% yield). LCMS (220 nm, 254 nm): t_{R} 2.541 min, purity ≥95%, m/z (ESI): 308.30 [M+CH₃CN]⁺. ¹H NMR (500 MHz, DMSO-d₆) δ 12.55 (br s, 1H), 7.65 (d, J=8.0 Hz, 1H), 7.56 (s, 1H), 7.29 (dd, J=11.4, 8.1 Hz, 1H), 7.19 (td, J=8.1, 5.3 Hz, 1H), 3.90 (s, 3H), 2.49 (s, 3H). ¹⁹F NMR (471 MHz, DMSO-d₆) δ-129.94 Hz.

### Synthesis of N-(2-bromo-3-methylphenyl)-5-(3-fluoro-2-methoxyphenyl)-N,3-dimethylthiophene-2-carboxamide

According to general procedure C for amide coupling. 5-(3-Fluoro-2-methoxyphenyl)-3-methylthiophene-2-carboxylic acid (94 mg, 0.35 mmol), 2-bromo-N,3-dimethylaniline (70 mg, 0.35 mmol), DMF (1 mL). Purification using preparative HPLC Method B. Solvent was concentrated under vacuo to give the desired product as a brownish oil (40 mg, 25% yield). LCMS (220 nm, 254 nm): t_{R} 3.257 min, m/z (EI): 448.10 [M]⁺.

### Synthesis of 5-{2-methyl-6-[N-methyl-5-(3-fluoro-2-methoxyphenyl)-3-methylthiophene-2-amido]phenyl}thiophene-2-carboxylic acid (RU-NT-518)

According to the general procedure for Suzuki coupling reactions using Method A, precatalyst C1 and saponification of methyl esters. N-(2-bromo-3-methylphenyl)-5-(3-fluoro-2-methoxyphenyl)-N,3-dimethylthiophene-2-carboxamide (40 mg, 0.09 mmol), [5-(methoxycarbonyl)thiophen-2-yl]boronic acid (33.19 mg, 0.18 mmol), dry dioxane (1 mL), temperature: 100° C. The brownish oil crude obtained after the workup was taken directly for the saponification. Purification using preparative HPLC Method B. Solvent was concentrated under vacuo to give the desired product was obtained as a white solid (6 mg, 14% yield). LCMS (220 nm, 254 nm): t_{R} 2.927 min, purity ≥95%, m/z (ESI): 496.20 [M+H]⁺. ¹H NMR for the observed rotamers (500 MHz, DMSO-d₆) δ 13.15 (s, 1H), 7.73 (d, J=3.7 Hz, 1H), 7.56 (d, J=7.9 Hz, 1H), 7.51-7.29 (m, 3H), 7.29-7.08 (m, 2H), 6.87 (s, 1H), 3.10 (s, 3H), (m, 6H, beneath the DMSO peak), 2.19 (s, 3H).

### Synthesis of N-(2-bromo-3-methylphenyl)cyclohexanecarboxamide

According to general procedure C for amide coupling. 2-Bromo-3-methylaniline (0.46 mL, 3.43 mmol), cyclohexanecarboxylic acid (400 mg, 3.12 mmol), EtOAc (3 mL). Purification using preparative HPLC Method B. Solvent was concentrated under vacuo to give the desired product was obtained as an off-white solid (490 mg, 53% yield). LCMS (220 nm, 254 nm): t_{R} 2.877 min, purity ≥95%, m/z (EI): 296.10 [M]⁺.

### Synthesis of methyl 5-(2-cyclohexaneamido-6-methylphenyl)thiophene-2-carboxylate

According to the general procedure for Suzuki coupling reactions using Method A, precatalyst C1. N-(2-bromo-3-methylphenyl)cyclohexanecarboxamide (300 mg, 1.01 mmol), [5-(methoxycarbonyl)thiophen-2-yl]boronic acid (207.21 mg, 1.11 mmol), dry dioxane (13 mL), temperature: 100° C. Purification using preparative HPLC Method B. Solvent was concentrated under vacuo to give the desired product was obtained as a white solid (120 mg, 33% yield). LCMS (220 nm, 254 nm): t_{R} 2.830 min, purity ≥95%, m/z (ESI): 358.15 [M+H]⁺. ¹H NMR (500 MHz, DMSO-d₆) δ 8.91 (s, 1H), 7.82 (d, J=3.7 Hz, 1H), 7.31 (d, J=7.7 Hz, 1H), 7.26 (dd, J=8.1, 1.4 Hz, 1H), 7.20 (d, J=7.4 Hz, 1H), 7.01 (d, J=3.8 Hz, 1H), 3.84 (s, 3H), 2.14 (s, 3H), 2.13-2.10 (m, 1H), 1.66-1.59 (m, 2H), 1.60-1.49 (m, 3H), 1.34-0.91 (m, 5H).

### Synthesis of methyl 5-[2-methyl-6-(N-methylcyclohexaneamido)phenyl]thiophene-2-carboxylate

According to the general procedure for N-alkylation of amides (Method B). Methyl 5-(2-cyclohexaneamido-6-methylphenyl)thiophene-2-carboxylate (120 mg, 0.34 mmol). Extraction was carried out 3x with DCM and brine, the organic layer was combined and dried over anhydrous MgSO₄. Solvent was concentrated under vacuo and the crude yellow oil was taken directly for the saponification (120 mg, 96% yield). LCMS (220 nm, 254 nm): t_{R} 2.997 min, m/z (ESI): 372.10 [M+H]⁺.

### Synthesis of 5-[2-methyl-6-(N-methylcyclohexaneamido)phenyl]thiophene-2-carboxylic acid (RU-NT-519)

According to the general procedure for saponification of methyl esters. Methyl 5-[2-methyl-6-(N-methylcyclohexaneamido)phenyl]thiophene-2-carboxylate (120 mg, 0.32 mmol). Purification using preparative HPLC Method B. Solvent was concentrated under vacuo to give the desired product was obtained as a white solid (62 mg, 54% yield). LCMS (220 nm, 254 nm): t_{R} 2.580 min, purity ≥95%, m/z (ESI): 358.15 [M+H]⁺. ¹H NMR for the observed rotamers (500 MHz, DMSO-d₆) δ 13.16 (s, 1H), 7.90-7.58 (m, 1H), 7.58-7.33 (m, 2H), 7.29 (dd, J=7.5, 1.4 Hz, 1H), 7.09 (d, J=3.7 Hz, 1H), 2.90 (s, 3H), 2.21 (s, 3H), 1.97 (tt, J=11.5, 3.5 Hz, 1H), 1.57 (ddd, J=43.1, 29.8, 12.3 Hz, 4H), 1.34-0.93 (m, 5H), 0.83 (tdd, J=12.8, 9.0, 3.4 Hz, 1H).

### Synthesis of N-(2-bromo-3-methylphenyl)-2-methylbenzamide

According to general procedure C for amide coupling. 2-Bromo-3-methylaniline (0.43 mL, 1.1 eq), 2-methylbenzoic acid (400 mg, 2.94 mmol), EtOAc (3 mL). Purification using preparative HPLC Method B. Solvent was concentrated under vacuo to give the desired product was obtained as beige crystals (280 mg, 31% yield). LCMS (220 nm, 254 nm): t_{R} 2.732 min, purity ≥95%, m/z (ESI): 305.15 [M+H]⁺. ¹H NMR (500 MHz, DMSO-d₆) δ 9.88 (s, 1H), 7.56 (d, J=7.5 Hz, 1H), 7.39 (td, J=7.5, 1.5 Hz, 3H), 7.31 (dq, J=7.7, 4.3 Hz, 1H), 7.27 (dd, J=7.6, 1.8 Hz, 1H), 2.45 (s, 3H), 2.41 (s, 3H).

### Synthesis of methyl 5-[2-methyl-6-(2-methylbenzamido)phenyl]thiophene-2-carboxylate

According to the general procedure for Suzuki coupling reactions using Method A, precatalyst C1. N-(2-bromo-3-methylphenyl)-2-methylbenzamide (250 mg, 0.82 mmol), [5-(methoxycarbonyl)thiophen-2-yl]boronic acid (168 mg, 1.1 eq), dry dioxane (11 mL), temperature: 100° C. Purification using preparative HPLC Method B. Solvent was concentrated under vacuo to give the desired product was obtained as a clear oil after further dryness on the V-10 (190 mg, 63% yield). LCMS (220 nm, 254 nm): t_{R} 2.753 min, purity ≥95%, m/z (ESI): 366.15 [M+H]⁺. ¹H NMR (500 MHz, DMSO-d₆) δ 9.61 (s, 1H), 7.83 (d, J=3.7 Hz, 1H), 7.41 (t, J=7.7 Hz, 1H), 7.35 (d, J=7.8 Hz, 1H), 7.30 (td, J=7.3, 1.6 Hz, 2H), 7.24-7.18 (m, 2H), 7.18-7.13 (m, 1H), 7.10 (d, J=3.8 Hz, 1H), 3.83 (s, 3H), 2.19 (s, 3H), 2.18 (s, 3H).

### Synthesis of 5-[2-methyl-6-(N-methyl-2-methylbenzamido)phenyl]thiophene-2-carboxylic acid (RU-NT-520)

According to the general procedure for N-alkylation of amides (Method B) and saponification of methyl esters. Methyl 5-[2-methyl-6-(2-methylbenzamido)phenyl]thiophene-2-carboxylate (190 mg, 0.52 mmol). Extraction was carried out 3x with EtOAc and brine, the organic layer was combined and dried over anhydrous MgSO₄. Solvent was concentrated under vacuo and the crude yellow oil was taken directly for the saponification. Purification using preparative HPLC Method B. Solvent was concentrated under vacuo to give the desired product was obtained as a white solid (42 mg, 22% yield). t_{R} 2.359 min, purity ≥95%, m/z (ESI): 366.10 [M+H]⁺. ¹H NMR for the observed rotamers (500 MHz, DMSO-d₆) δ 13.10 (s, 1H), 7.77 (dd, J=12.5, 3.8 Hz, 1H), 7.49 (t, J=7.8 Hz, 1H), 7.42-7.21 (m, 4H), 7.11 (d, J=3.7 Hz, 2H), 6.95-6.73 (m, 2H), 2.82 (s, 3H), 2.18 (s, 3H), 2.04 (s, 3H).

### Synthesis of methyl 2-[(2-bromo-3-methylphenyl)carbamoyl]benzoate

According to general procedure C for amide coupling. 2-Bromo-3-methylaniline (0.41 mL, 1.1 eq), 2-(methoxycarbonyl)benzoic acid (500 mg, 2.78 mmol), EtOAc (3 mL). Purification using preparative HPLC Method B. Solvent was concentrated under vacuo to give the desired product was obtained as a white solid (320 mg, 33% yield). LCMS (220 nm, 254 nm): t_{R} 2.576 min, purity=71%, m/z (ESI): 412.30 [M+CH₃CN+Na]⁺.

### Synthesis of methyl 5-{2-[2-(methoxycarbonyl)benzamido]-6-methylphenyl}thiophene-2-carboxylate

According to the general procedure for Suzuki coupling reactions using Method A, precatalyst C1. Methyl 2-[(2-bromo-3-methylphenyl)carbamoyl]benzoate (300 mg, 0.86 mmol), [5-(methoxycarbonyl)thiophen-2-yl]boronic acid (320 mg, 1.72 mmol), dry dioxane (11 mL), temperature: 100° C. Purification using preparative HPLC Method B. Solvent was concentrated under vacuo to give the desired product was obtained as a yellow oil after dryness on the V-10 (130 mg, 37% yield). LCMS (220 nm, 254 nm): t_{R} 2.723 min, purity ≥95%, m/z (ESI): 473.15 [M+CH₃CN+Na]⁺.

### Synthesis of methyl methyl 5-{2-methyl-6-[N-methyl-2-(methoxycarbonyl)benzamido]phenyl}thiophene-2-carboxylate

According to the general procedure for N-alkylation of amides (Method B). Methyl 5-{2-methyl-6-[N-methyl-2-(methoxycarbonyl)benzamido]phenyl}thiophene-2-carboxylate (130 mg, 0.31 mmol). Purification using preparative HPLC Method B. Solvent was concentrated under vacuo to give the desired product was obtained as a clear oil after dryness on the V-10 (50 mg, 39% yield). LCMS (220 nm, 254 nm): t_{R} 2.693 min, purity ≥95%, m/z (ESI): 424.20 [M+H]⁺.

### Synthesis of 2-({2-[5-(methoxycarbonyl)thiophen-2-yl]-3-methylphenyl}(methyl)carbamoyl)benzoic acid (RU-NT-521) & 5-{2-methyl-6-[N-methyl-2-(methoxycarbonyl)benzamido]phenyl}thiophene-2-carboxylic acid (RU-NT-522)

According to the general procedure for saponification of methyl esters. The reaction mixture was stirred at rtp for 2-3 hours with 1eq of LiOH used. Reaction was monitored by LCMS. Methyl 5-{2-methyl-6-[N-methyl-2-(methoxycarbonyl)benzamido]phenyl}thiophene-2-carboxylate (50 mg, 0.12 mmol). Purification using preparative HPLC Method B, 200×30 mm size column. The desired products were obtained as white solids. 5-{2-Methyl-6-[N-methyl-2-(methoxycarbonyl)benzamido]phenyl}thiophene-2-carboxylic acid (3.6 mg, 7% yield), t_{R} 2.365 min, purity ≥95%, m/z (ESI): 410.15 [M+H]⁺. ¹H NMR for the observed rotamers (500 MHz, DMSO-d₆) δ 13.34 (s, 1H), 8.23-7.84 (m, 1H), 7.84-7.62 (m, 1H), 7.60-7.42 (m, 1H), 7.46-7.23 (m, 2H), 7.23-7.05 (m, 1H), 6.79 (ddd, J=98.9, 7.6, 1.3 Hz, 2H), 3.85 (d, J=15.6 Hz, 3H), 2.70 (s, 3H), 2.22 (s, 3H). 2-({2-[5-(Methoxycarbonyl)thiophen-2-yl]-3-methylphenyl}(methyl)carbamoyl)benzoic acid (5 mg, 10% yield). LCMS (220 nm, 254 nm): t_{R} 2.281 min, purity ≥95%, m/z (ESI): 410.15 [M+H]⁺. ¹H NMR for the observed rotamers (500 MHz, DMSO-d₆) δ 13.22 (br s, 1H), 7.97 (d, J=7.8 Hz, 1H), 7.87-7.67 (m, 1H), 7.67-7.49 (m, 2H), 7.39 (t, J=7.2 Hz, 2H), 7.25-6.79 (m, 2H), 6.67 (d, J=7.5 Hz, 1H), 3.90 (s, 3H), 2.76 (s, 3H), 2.22 (s, 3H).

### Synthesis of methyl 3-[(2-bromo-3-methylphenyl)carbamoyl]benzoate

According to general procedure C for amide coupling. 2-Bromo-3-methylaniline (0.32 mL, 1.1 eq), 3-(methoxycarbonyl)benzoic acid (400 mg, 1 eq), EtOAc (3 mL). Extraction was carried out 3x with EtOAc and brine. The organic layer was combined and dried over anhydrous MgSO₄ and solvent was reduced under vacuo to give the desired product as white crystals. LCMS (220 nm, 254 nm): t_{R} 2.653 min, purity ≥95%, m/z (ESI): 410.15 [M+H]⁺. ¹H NMR (500 MHz, DMSO-d₆) δ 10.32 (s, 1H), 8.58 (s, 1H), 8.45-8.22 (m, 1H), 8.17 (dt, J=7.8, 1.5 Hz, 1H), 7.71 (t, J=7.8 Hz, 1H), 7.51-7.15 (m, 3H), 3.91 (s, 3H), 2.42 (s, 3H).

### Synthesis of methyl 5-{2-[3-(methoxycarbonyl)benzamido]-6-methylphenyl}thiophene-2-carboxylate

According to the general procedure for Suzuki coupling reactions using Method A, precatalyst C1. Methyl 3-[(2-bromo-3-methylphenyl)carbamoyl]benzoate (500 mg, 1.44 mmol), [5-(methoxycarbonyl)thiophen-2-yl]boronic acid (294 mg, 1.58 mmol), dry dioxane (15 mL), temperature: 100° C. Purification using preparative HPLC Method B. Solvent was concentrated under vacuo to give the desired product as a crude yellow oil (250 mg, 43% yield). LCMS (220 nm, 254 nm): t_{R} 2.735 min, m/z (ESI): 381.10 [M+CH₃CN]⁺.

### Synthesis of methyl 5-{2-methyl-6-[N-methyl-3-(methoxycarbonyl)benzamido]phenyl}thiophene-2-carboxylate

According to the general procedure for N-alkylation of amides (Method B). Methyl 5-{2-[3-(methoxycarbonyl)benzamido]-6-methylphenyl}thiophene-2-carboxylate (250 mg, 0.61 mmol). Purification using preparative HPLC Method B. Solvent was concentrated under vacuo to give the desired product as a clear oil (40 mg, 16% yield). LCMS (220 nm, 254 nm): t_{R} 2.723 min, purity ≥95%, m/z (ESI): 424.10 [M+H]⁺.

### Synthesis of 5-[2-methyl-6-(N-methyl-3-carboxybenzamido)phenyl]thiophene-2-carboxylic acid (RU-NT-523)

According to the general procedure for saponification of methyl esters. Methyl 5-{2-methyl-6-[N-methyl-3-(methoxycarbonyl)benzamido]phenyl}thiophene-2-carboxylate (40 mg, 0.09 mmol). Purification using preparative HPLC Method B. Solvent was concentrated under vacuo to give the desired product as a white solid (12.77 mg, 34% yield). LCMS (220 nm, 254 nm): t_{R} 2.068 min, purity ≥95%, m/z (ESI): 396.15 [M+H]⁺. ¹H NMR for the observed rotamers (500 MHz, DMSO-d₆) δ 13.10 (br s, 1H), 8.14-7.75 (m, 1H), 7.72-7.57 (m, 2H), 7.59-7.06 (m, 4H), 6.70 (d, J=3.8 Hz, 1H), 3.08 (s, 3H), 2.15 (s, 3H).

### Example 2: Structure-Based Design of RTA Inhibitors

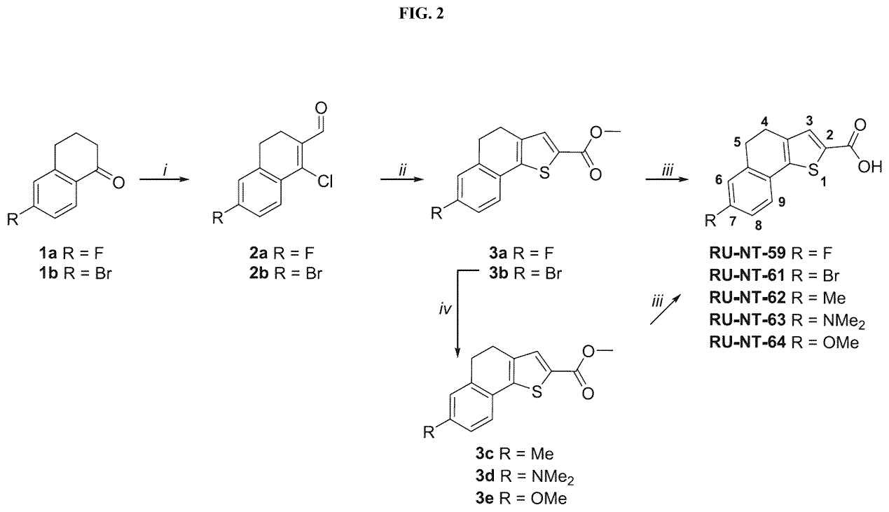

The X-ray crystal structure of the best inhibitor identified to date, RU-NT-93 in complex with RTA revealed that RU-NT-93 bound RTA in a unique manner where the thiophene ring and its carboxylate in RU-NT-93 were rotated by 770 making it perpendicular relative to the carboxylate moiety in CC10501 (FIG. 4). To determine if the orientation of the thiophene is important for increased affinity planar compounds where the molecule is locked in a fixed conformation were analyzed (Table 2). The constrained analog of CC10501, PD00589, was commercially available. RU-NT-59 and RU-NT-61 were synthesized via 3-step synthesis (FIG. 2). Ketones 1a and 1b underwent a Vilsmeier-Haack formylation to give aldehydes 2a and 2b, which subsequently were treated with ethyl thioglycolate to produce the cyclization product 3a and 3b. Afterward, esters 3a and 3b were converted to acids RU-NT-59 and RU-NT-61 respectively using 1 M sodium hydroxide in THF and methanol. Compounds 3c, 3d, and 3e were synthesized from 3b using palladium-catalyzed cross-coupling reactions and later were converted to respective acids RU-NT-62, RU-NT-63, and RU-NT-64 using the same conditions. The difference between PD00589 and RU-NT-59, RU-NT-61, RU-NT-62, RU-NT-63, and RU-NT-64 is that they all have different groups instead of hydrogen at the C7 position (FIG. 2).

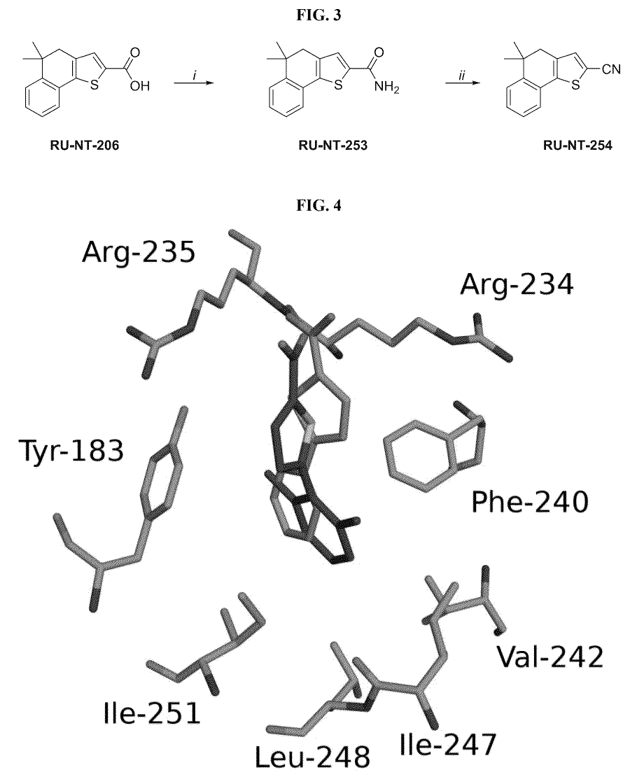

Additionally, RU-NT-206 was used to help evaluate substitution on the central ring and analogues thereof were prepared (FIG. 3). Amide RU-NT-253 was synthesized from acid RU-NT-206 via condensation with ammonium chloride in the presence of HATU and (i-Pr)₂NEt, and treatment of this amide with Burgess' reagent (methyl N-(triethylammoniumsulfonyl) carbamate) gave nitrile RU-NT-254.

TABLE 2
Chemical structures, affinity, and inhibitory activity of certain exemplary
compounds

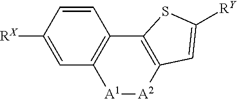

| Cmpd | R^{X} | A¹ | A² | R^{Y} | Kᵢ (μM) | IC₅₀ (μM) |
|---|---|---|---|---|---|---|
| RU-NT-206 | H | CMe₂ | CH₂ | COOH | 1 | 7 |
| RU-NT-253 | H | CMe₂ | CH₂ | C(O)NH₂ | U.D. | U.D. |
| RU-NT-254 | H | CMe₂ | CH₂ | CN | U.D. | U.D. |
| RU-NT-59 | 7-F | CH₂ | CH₂ | COOH | 4 | 17 |
| PD00589 | H | CH₂ | CH₂ | COOH | 8 | 30 |
| RU-NT-62 | 7-Me | CH₂ | CH₂ | COOH | 18 | 70 |
| RU-NT-61 | 7-Br | CH₂ | CH₂ | COOH | 23 | 90 |
| RU-NT-64 | 7-OMe | CH₂ | CH₂ | COOH | 43 | 166 |
| RU-NT-63 | 7-NMe₂ | CH₂ | CH₂ | COOH | U.D. | U.D. |
| PD00633 | H | CH₂ₚ | CH₂ | C(O)NHNH₂ | U.D. | U.D. |
| RU-NT-198 | H | CH₂ | O | COOH | 20 | 77 |
| SEW01765 | 8-F | O | CH₂ | COOMe | U.D. | U.D. |
| RU-NT-199 | 8-F | CH₂ | S | COOH | 28 | 107 |
| SEW01776 | 8-C1 | S | CH₂ | COOH | 15 | 59 |
| SEW01689 | 8-C1 | S | CH₂ | C(O)NHNH₂ | U.D. | U.D. |
| SEW02679 | 8-C1 | S | CH₂ | C(O)Me | U.D. | U.D. |
| RU-NT-201 | H | COOH | S | — | 94 | — |

### Example 3: Affinity and Potency of Exemplary Inhibitors of the Disclosure

To characterize compounds that disrupt the interaction between RTA and the P-stalk a fluorescence polarization (FP) competition assay was developed. The FP competition assay measures the decrease in FP caused by inhibitors that displace the BODIPY TMR-X NHS labeled P11 peptide probe from RTA. First, the optimal concentration of the fluorescent P11 was determined by measuring the FP as a function of its concentration. In the second step, binding of the fluorescently labeled P11 to purified recombinant RTA was determined by dose titration of the fluorescent P11 with varying concentrations of RTA at a fixed concentration of P11 (1 μM). The FP increased with increasing concentrations of RTA, indicating the binding of BODIPY TMR-X-labeled P11 to RTA. The fluorescently labeled P11 was nearly fully bound to RTA with a KD value of 1±0.2 μM (FIG. 5A). The specificity of the assay was established by competition with unlabeled P11 as a positive control and by competition with PT peptide, which binds at the active site of RTA and BTB13068, a small molecule, which binds RTA remote from the P-stalk site, as negative controls. The IC₅₀ value determined by the FP competition assay is the concentration of the inhibitor required to replace 50% of the fluorescent P11 probe from RTA. The IC₅₀ values obtained from the FP competition assay were used to determine the Kᵢ values as described herein.

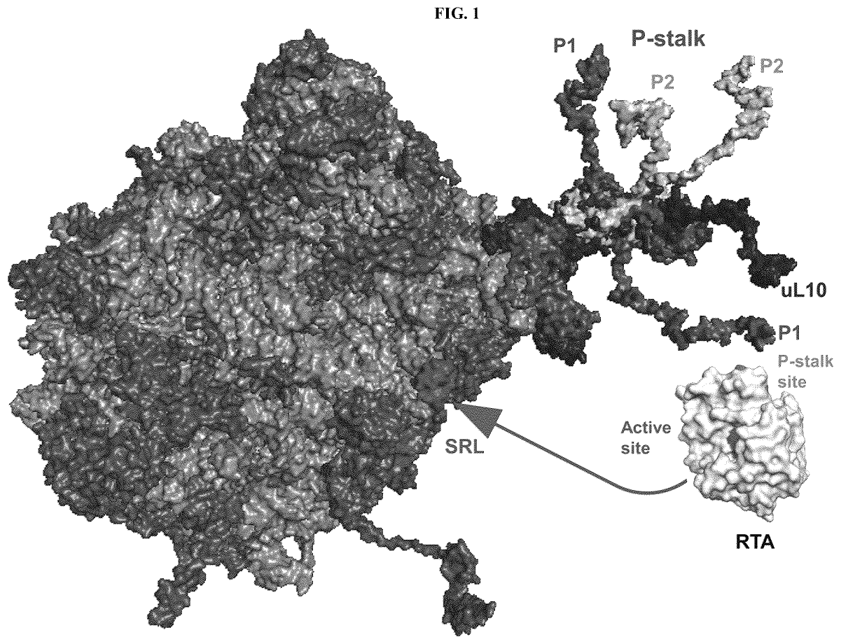

The inhibitory constant, Kᵢ, reflects the affinity of the inhibitor and is defined as the concentration of the inhibitor that will bind to half of the binding sites on RTA at equilibrium in the absence of labeled P11. As the concentration of unlabeled P11 increased, the labeled probe was displaced from RTA with an IC₅₀ value of 4±1 μM and a Kᵢ value of 1'0.3 μM, indicating that the unlabeled P11 can competitively displace the labeled probe (FIG. 6A). The PT peptide (FIG. 5B) or BTB13068 (FIG. 5C) did not show any effect, providing evidence that the binding was not due to nonspecific hydrophobic interactions with RTA. The KD value (1 μM) of the labeled P11 obtained by the direct binding assay (FIG. 5A) was the same as the Kᵢ value of the unlabeled P11 obtained by the competition assay (1 μM) (FIG. 1), indicating that there was no interference from the BODIPY-TMR-X dye attached to the P11 peptide and that the interaction between P11 and RTA is specific.

### Example 4: Structure-Activity Relationship of Exemplary Inhibitors of the Disclosure

Compound RU-NT-206 (entry 1, Table 2), which has a Kᵢ of 1 μM and is 8-fold better than that observed for PD00589, which lacks the gem-dimethyl substitution. The results indicated that the presence of carboxylic acid is essential for improved affinity. Replacement with nitrile, hydrazide, ester, or ketone completely removes activity (entries 3, 10 and 15, 12, and 16, respectively, Table 2). Substitution at the C7-position with a fluoride doubled the potency compared to no substituents (entry 4 vs 5, Table 2). Increasing the size of the substituent appears to reduce the activity (entries 5-9, Table 2). It is not clear if the basicity of RU-NT-63 or the size of the dimethylamine was responsible for the lack of activity. Based on the X-ray structures of the analogs RU-NT-59, PD00589, and RU-NT-206, the pocket where the substituents at position 7 are pointing is highly hydrophobic which may provide an alternative explanation as to why more polar substituents have lower affinity. Several compounds were evaluated and it was found that the substitution of the 4-methylene group by an oxygen atom (RU-NT-198, R1=H) or sulfur (RU-NT-199, R1=8-F) was tolerated with ~5 to 7-fold loss in potency. The activity of these backbone-changed compounds is similar to that of the benzo[b]thieno2-thiophene RU-NT-201 where both methylenes (Table 2) are replaced by a single sulfur. RU-NT-93, PD00589, RU-NT-59, and RU-NT-206 displaced the labeled P11 probe from RTA with 7-, 8-, 13- and 32-fold improved IC₅₀ values relative to CC10501, respectively (FIGS. 6B-6F and Table 3).

TABLE 3
The Kᵢ, in vitro IC₅₀ measured by the FP and qRT-PCR assay, and the EC₅₀ for
the depurination in cell-based assays by certain exemplary CC10501 analogs

| | | IC₅₀ (μM) | | % Inh (Vero) | | % Inh (A549) | | EC₅₀ |
|---|---|---|---|---|---|---|---|---|
| | | | qRT- | 500 | 250 | 500 | 250 | (μM) |
| Cmpd | Kᵢ (μM) | FP | PCR | μM | μM | μM | μM | (Vero) |
| CC10501 | 58 ± 0.3 | 224 ± 1 | 408 ± 54 | 0 | 0 | U.D. | U.D. | U.D. |
| RU-NT-93 | 8 ± 0.3 | 31 ± 1 | 45 ± 5 | 66 ± 8 | 52 ± 5 | 69 ± 7 | 59 ± 9 | U.D. |
| PD00589 | 8 ± 0.3 | 30 ± 1 | 28 ± 4 | 75 ± 7 | 61 ± 10 | 69 ± 15 | 53 ± 2 | U.D. |
| RU-NT-59 | 4 ± 0.3 | 17 ± 1 | 32 ± 4 | 84 ± 5 | 62 ± 5 | 69 ± 7 | 51 ± 8 | U.D. |
| RU-NT- | 1 ± 0.3 | 7 ± 1 | 23 ± 4 | 95 ± 3 | 87 ± 7 | 86 ± 5 | 75 ± 8 | 29 ± 2 |
| 206 | | | | | | | | |
| P11 | 1 ± 0.3 | 4 ± 1 | 31 ± 6 | U.D. | U.D. | U.D. | U.D. | U.D. |

CC10501 had a calculated Kᵢ value of 58 μM (FIG. 6B and Table 3). RUNT-93, PD00589, and RU-NT-59 had 7-, 7-, and 15-fold improved Kᵢ values relative to CC10501 respectively (FIGS. 6C-6F and Table 3). RUNT-206 had the highest affinity for RTA with ~60-fold improved Kᵢ of 1 μM (FIG. 6F and Table 3), which was identical to the affinity of the P11 peptide for RTA (FIG. 6A and Table 3).

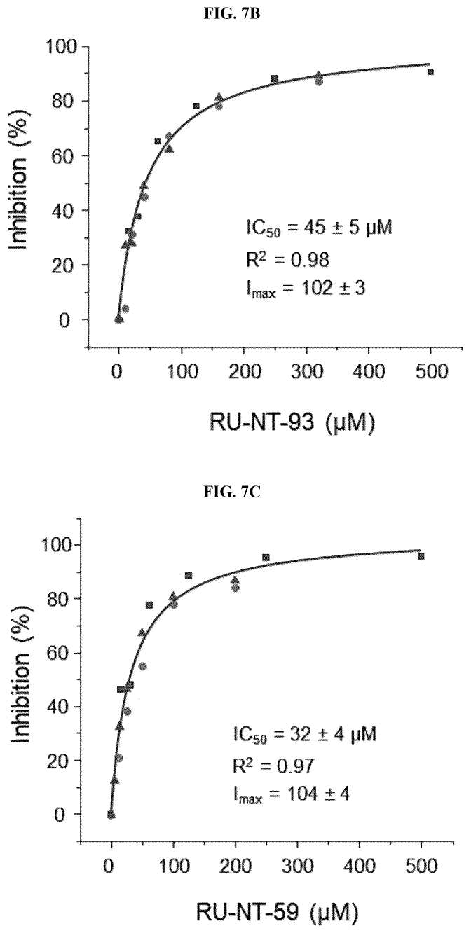

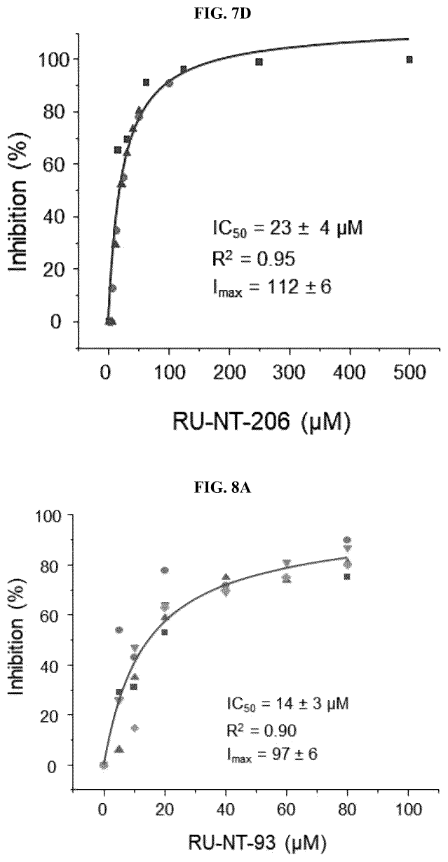

The IC₅₀ values were also determined by measuring the inhibition of RTA-mediated depurination of rat liver ribosomes by RTA using the qRT-PCR assay. The data for the percent inhibition at different compound concentrations were fitted with Michaelis-Menten kinetics using OriginPro (FIGS. 7A-7D). The IC₅₀ value of CC10501 by the qRT-PCR assay was determined by linear regression analysis because depurination increased linearly and at 500 μM 64% inhibition was obtained. The IC₅₀ values for RU-NT-93, PD00589, RU-NT-59, and RU-NT-206 by qRT-PCR improved 9-, 15-, 13-, and 18-fold relative to CC10501, respectively using rat liver ribosomes (Table 3 and FIGS. 7A-7D). RU-NT-206 showed the greatest potency with an IC₅₀ value of 23 μM by the qRT-PCR assay (FIG. 7D). The IC₅₀ values determined by the FP competition, and the qRT-PCR assay were in similar rank order and were proportional to the K, values (Table 3), indicating that compounds optimized for binding at the P-stalk pocket more potently inhibit the activity of RTA.

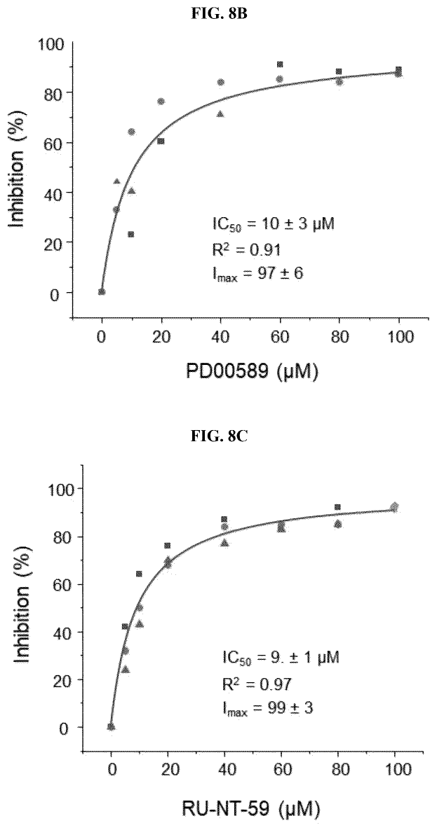

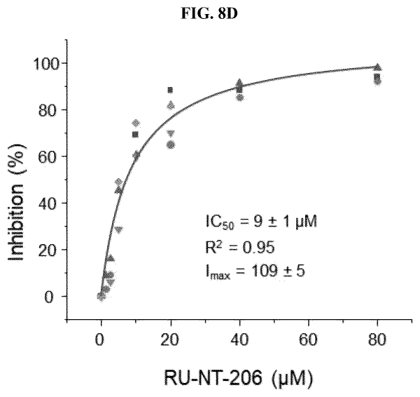

The IC₅₀ values were determined by qRT-PCR using yeast ribosomes as well. They were about 3-fold lower than the IC₅₀ values obtained with rat liver ribosomes (FIGS. 8A-8D) since yeast ribosomes are less sensitive to RTA than mammalian ribosomes. The previously identified inhibitor, RU-NT-93 had a 10-fold improved IC₅₀ while PD00589 and RU-NT-59 had 15- and 16-fold improved IC₅₀ values of 10 and 9 μM, respectively relative to CC10501 using yeast ribosomes (FIGS. 8A-8C). The IC₅₀ value for RU-NT-206 showed a 17-fold improvement compared to CC10501 with yeast ribosomes (FIG. 8D). The IC₅₀ values of RU-NT-206 measured by qRT-PCR using yeast and rat liver ribosomes (9 μM and 23 μm, respectively) were in close agreement with the IC₅₀ values of P11 peptide using yeast or rat liver ribosomes (5 μM and 31 μM, respectively). The Kᵢ and IC₅₀ values of P11 measured by the FP competition assay were also similar to RU-NT-206 (Table 3). These results demonstrate that RU-NT-206 shows a similar affinity and inhibitory potency against RTA as the 5-fold larger P11 peptide.

### Example 5: Structure of RTA Inhibitor Complexes

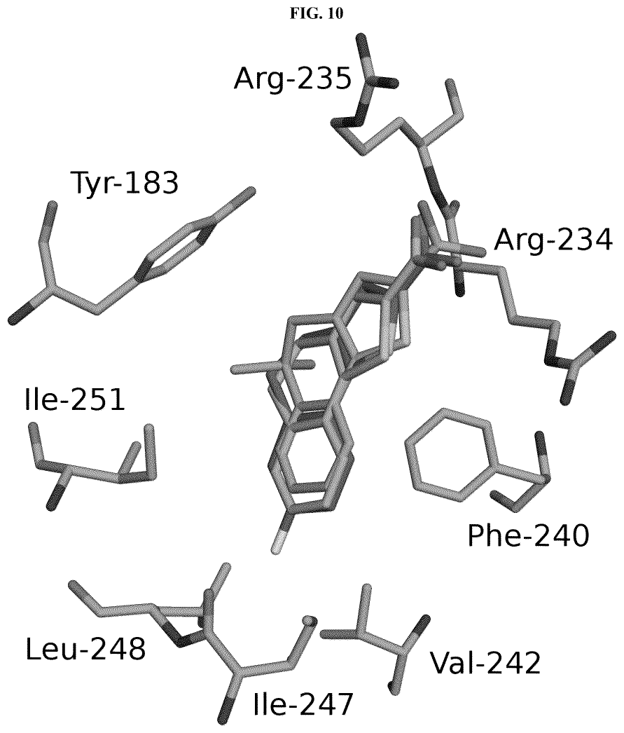

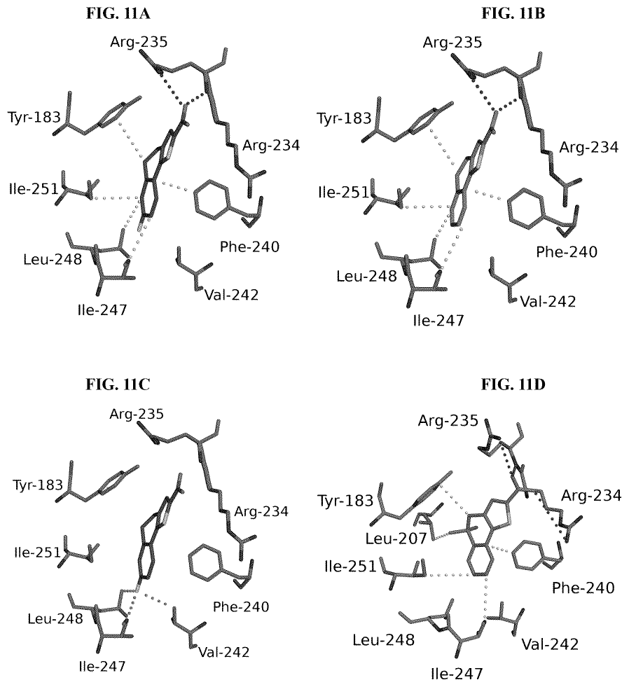

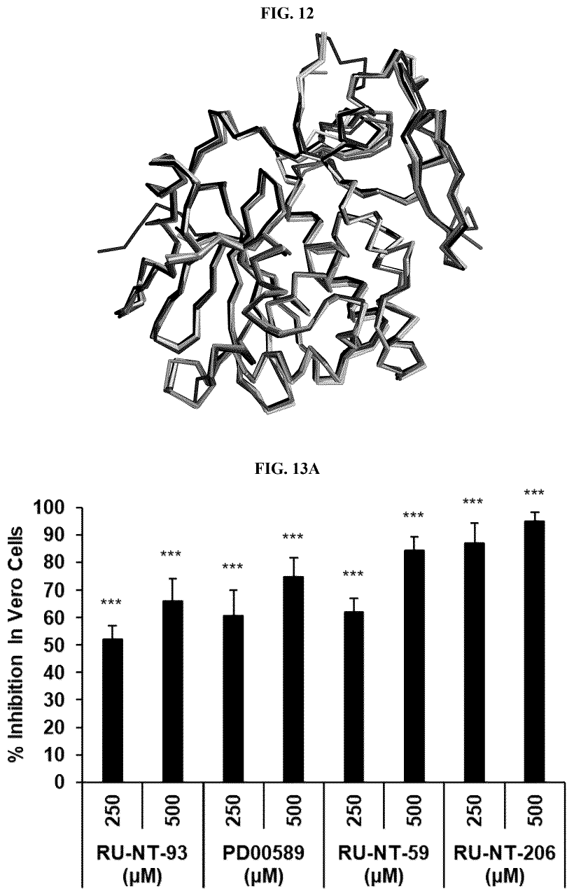

RTA was cocrystallized with RU-NT-59, PD00589, and RU-NT-206 and each RTA-inhibitor complex structure was solved by molecular replacement using PHASER (FIGS. 9A-9C). The RTA-RU-NT-59 complex was determined at 1.9 Å, the RTA-PD00589 structure was solved at 2.3 Å, and the RTA-RU-NT-206 complex was solved at 2.7 Å. Each structure was determined in the hexagonal P6322 space group. The electron density for each inhibitor was well defined in each structure (FIGS. 9D-9F). The location of RU-NT-59, PD00589, and RU-NT-206 bound within the P-stalk pocket of RTA is very similar (FIG. 10). The thiophene carboxylate in each inhibitor shared a salt-bridge with the side chain of Arg235 along with a hydrogen bond interaction with the main chain amide nitrogen of Arg235 (FIGS. 11A-11B and FIG. 11D). The cyclohexene and benzene rings in all three compounds formed similar hydrophobic interactions with Tyr183 and Phe240 in RTA. Each benzene ring in RU-NT-59 and PD00589 also hydrophobically contacted I1e247, Leu248, and I1e251 (FIGS. 11A-11B and FIG. 11D) with the fluoride atom of the fluorobenzene ring of RU-NT-59 additionally contacting Val242 in RTA while also making much closer hydrophobic contacts with I1e247 and Leu248 (FIG. 11C).

Although each inhibitor was similarly bound to the P-stalk pocket, RU-NT-206 was positioned ~1 Å closer to Arg234 and Arg235. Consequently, the thiophene carboxylate in RU-NT-206 formed an additional salt bridge with Arg234 (FIG. 11D). In addition, the dihedral angle between the carboxylate group and the thiophene ring is 43.2° in RU-NT-206. This conformational twist results in reduced conjugation and increased electron density on the carboxylate, which increases the ionic interactions with the positively charged arginines. The benzene ring in RU-NT-206 hydrophobically interacted with I1e247 and I1e251. The methyl group attached to position C5 in the cyclohexadiene ring of RU-NT-206 formed a unique hydrophobic contact with Leu207, a nonpolar residue that lined a hydrophobic cavity within the P-stalk pocket (FIG. 11D). The previously determined crystal structures of the RTA alone (PDB ID: 1RTC), RTA bound to the P11 peptide (PDB ID: 5GU4), or RTA bound to RU-NT-93 (PDB ID: 7MLP) are very similar to the RTA structures reported here. The RMSD range after the superposition of these structures ranged from 0.17 to 0.87 Å, indicating no large conformational changes in the backbone of RTA upon the binding of these inhibitors (FIG. 11D).

### Example 6: Protection against ricin and Stx2a holotoxin in cell-based assays

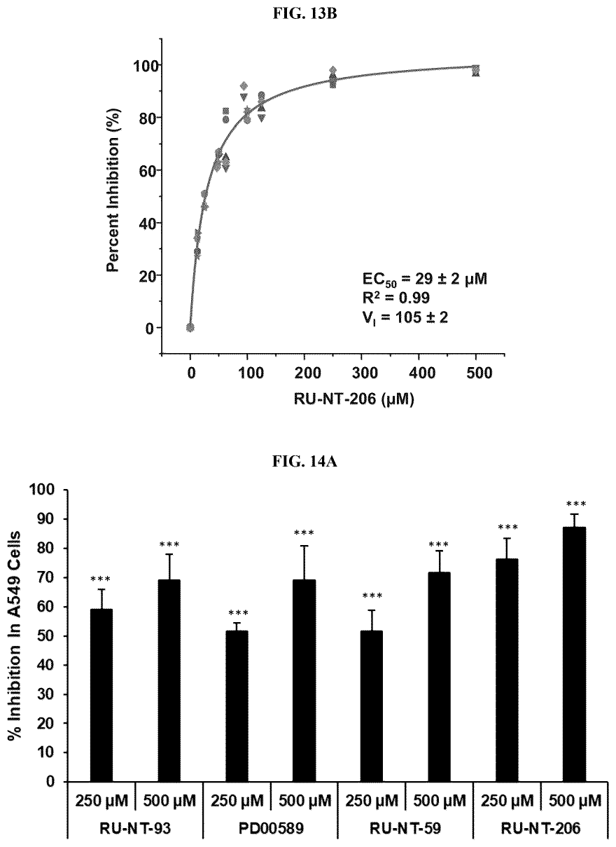

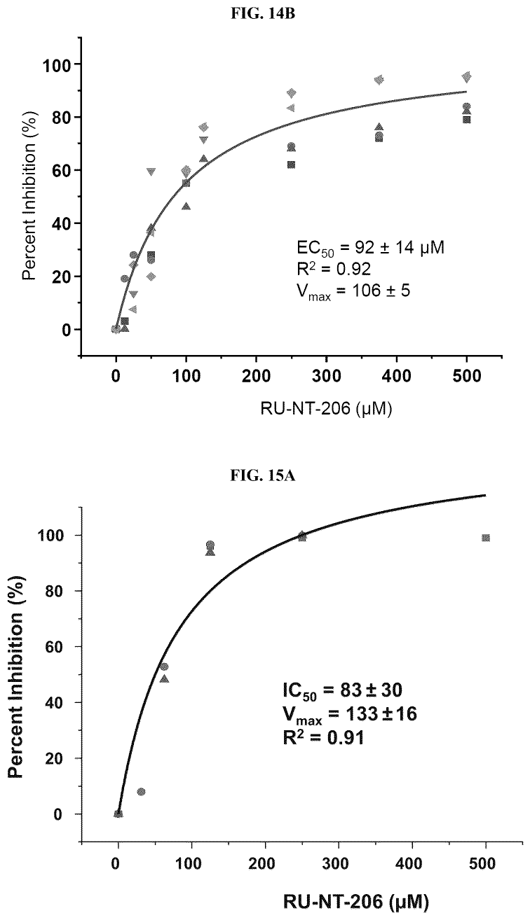

Structure-based optimization led to inhibitors with submicromolar binding affinity and inhibitory potency against depurination by RTA. To evaluate their cellular protection activity, cultured Vero cells were used to determine if compounds protected cells from depurination by ricin holotoxin. In the presence of 200 μM ricin, cellular ribosome depurination increased linearly for about 4 h. A two hour period of depurination by ricin was selected to compare the compounds. Two hours after the simultaneous addition of the toxin and the compound, cellular RNA was isolated and used for qRT-PCR to determine the level of depurination. RNA from cells treated with the same buffer but without ricin and compound was used as a negative control to represent full protection (100% inhibition). RNA from cells treated with ricin but no compound was used as a positive control (0% inhibition). Cells treated with 250 μM PD00589 resulted in a 61% inhibition of ribosome depurination and 75% inhibition at 500 μM (FIG. 13A and Table 3). Cells treated with RU-NT-59 showed 62% inhibition at 250 μM and 84% inhibition at 500 μM. The greatest protection was observed with RU-NT-206, exhibiting 87% inhibition at 250 μM and 95% at 500 μM. The EC₅₀ value of depurination inhibition for RU-NT-206 was determined by incubation with a titration series up to 500 μM. The half-maximal effective concentration (EC₅₀) of RU-NT-206 for inhibition of depurination by ricin holotoxin in Vero cells was 29±2 μM (FIG. 13B). These results indicate that the improved affinity and in vitro potency are predictive of a protective effect in cells.

Since inhalation is the most efficient exposure route to ricin, the protective activity of the compounds against ricin was determined by challenging human lung epithelial A549 cells as well. Cells treated with 250 μM PD00589 resulted in a 53% reduction in ribosome depurination and a 69% reduction at 500 μM (Table 3). Cells treated with RU-NT-59 showed 51% inhibition at 250 μM and 69% inhibition at 500 μM. The greatest protection was observed for RU-NT-206 in A549 cells as in Vero cells, with 75% inhibition at 250 μM and 86% at 500 μM (FIG. 14A). RU-NT-206 protected A549 cells against ricin holotoxin with an EC₅₀ of 92±14 μM (FIG. 14B). These results demonstrated that protection from ricin holotoxin by RU-NT-206 was not restricted to a specific cell line. The crystal structure of Stx2a with P11 (PDB ID: 6X6H) showed that as observed with RTA (PDB ID: 5GU4), only the last six residues of P11 peptide bound in a shallow pocket on Stx2a. The P11 binding site on Stx2a was differently located relative to the P11-binding site on RTA. Although RTA and Stx2a bind P-stalk peptides in a different manner, both pockets consist of positively charged and hydrophobic residues. Conserved hydrophobic residues (Leu9 and Phe10) and the last Asp (AspI1) of the P11 peptide play a critical role in ribosome binding to each toxin. Asp11 forms salt bridges and H-bonds with arginines at the P-stalk pocket of each toxin.

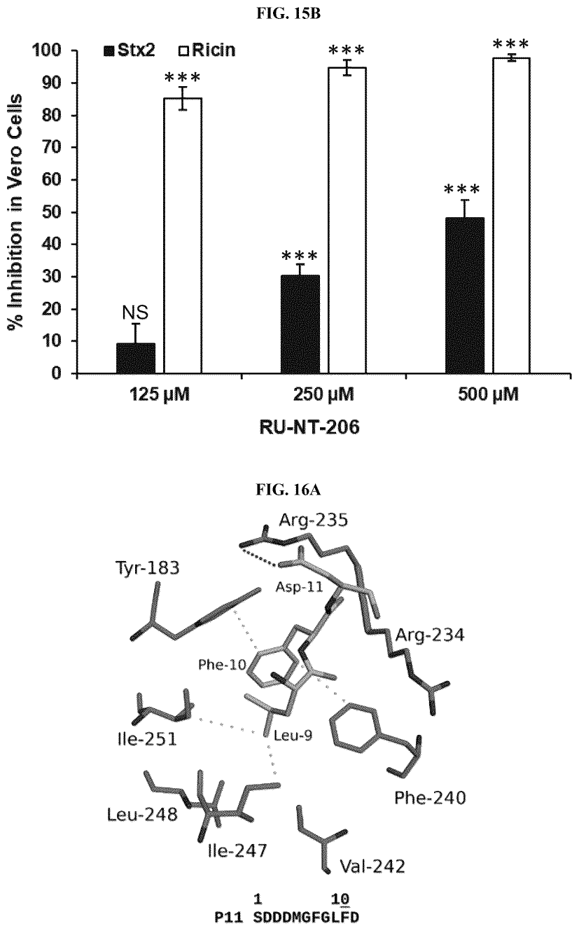

Because of these similarities, the effect of RU-NT-206 on the depurination activity of Stx2A1 using rat liver ribosomes was examined. RU-NT-206 protected rat liver ribosomes from depurination by Stx2A1 with an IC₅₀ value of 83±30 μM (FIG. 15A). Whether RU-NT-206 would protect against Stx2a holotoxin in Vero cells was evaluated. Two hours after the simultaneous addition of Stx2a and the compound, cellular RNA was isolated and used for qRT-PCR to determine the level of depurination. A comparison of protection by RU-NT-206 against the depurination activity of ricin and Stx2a holotoxins in Vero cells is shown in FIG. 14B. RU-NT-206 showed 9%, 30%, and 48% protection against Stx2a in Vero cells compared to 85%, 95% and 98% protection against ricin at 125, 250 and 500 μM, respectively. Although the level of protection against Stx2a was lower than ricin, it was significantly different from the control at 250 and 500 μM. These results identified RU-NT-206 as a lead small molecule with activity against both toxins.

### Example 7: Structure-Activity Relationship for Certain Exemplary RTA Inhibitors

Ricin and Stxs have been uniquely challenging drug targets. The catalytic site of ricin and Stx2a has been explored extensively as a potential target for antidotes. However, few inhibitors have been found, and none with activity in cells or in vivo. Although retrograde trafficking inhibitors have shown promising results, these inhibitors target the trafficking pathways, not the toxin itself, and may be detrimental to the host. Modulation of ribosome interactions by small molecules has not been fully explored as a strategy for the inhibition of ricin or Stx2a.

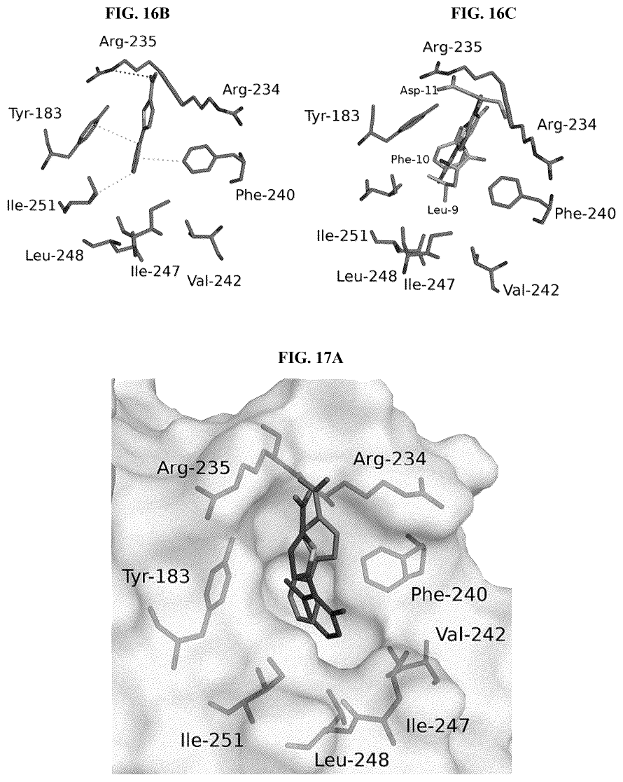

The crystal structure of RTA in complex with the P11 peptide showed that the C-terminal Asp11 within P11 contacts the two positively charged residues Arg234 and Arg235 in RTA forming a salt bridge with Arg235 (FIG. 16A) Leu9 in P11 makes hydrophobic interactions with several residues within the nonpolar region of the P11 pocket including Phe240, I1e247, and I1e251 while the aromatic side chain of the P11 residue Phe10 forms an offsetting 71-stack with RTA's Tyr183 and Phe240 (FIG. 16A). The crystal structure of RTA in complex with the RTA inhibitor CC10501 (PDB ID: 6URX) revealed that the carboxylate moiety of CC10501 salt-bridged with Arg235 within the P-stalk binding site of RTA as Asp11 in P11 (FIG. 16B). The benzene ring in CC10501, which superpositioned near the Phe10 side chain in P11, established similar π-stacking interactions with RTA's Tyr183 and Phe240 as Phe10 despite being offset by 560 with the side chain of Phe10 (FIG. 16C and FIG. 4). The benzene ring in CC10501 also made a comparable hydrophobic association with I1e251 as Phe10 did in P11 (FIGS. 16B-16C).

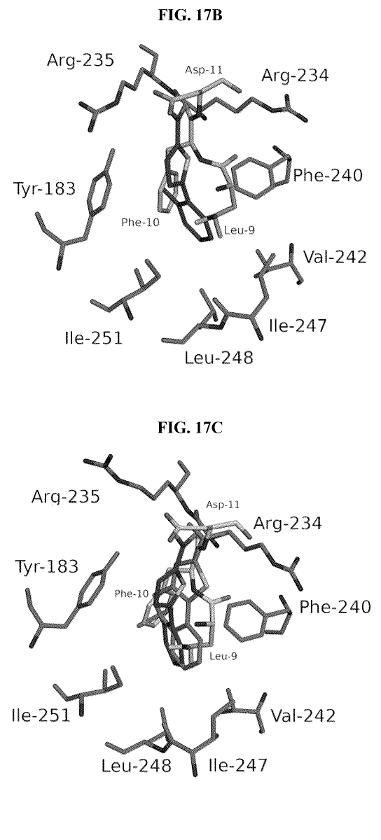

The lead, previously reported inhibitor (i.e., RU-NT-93) is distinct from CC10501, at least inasmuch as RU-NT-93 contains an m-xylene ring instead of the benzene ring in CC10501 (Table 3) and binds with a rotated thiophene ring and carboxylate relative to CC10501 (FIG. 4). The m-xylene ring in RU-NT-93 is positioned closer to the surface of the P stalk binding pocket relative to the benzene ring in CC10501 (FIG. 17A). Consequently, the methyl groups on the 2,6-dimethylphenyl ring of RU-NT-93 uniquely overlap with the space occupied by P11 residues Phe10 and Leu9 (FIG. 17B). The carboxylic acid of RU-NT-93 occupies the same space as the Asp11 side chain acid (FIG. 17B). CC10501 and RU-NT-93 bind the P11 pocket similarly (FIG. 17C). RU-NT-93 more completely fills the pocket compared to CC10501, indicating why RU-NT-93 is a more effective RTA inhibitor relative to CC10501 (FIG. 17C). The tighter binding of RU-NT-93 to RTA compared to CC10501 correlated well with the 7-9-fold higher RTA inhibition (Table 3) and over 10-fold greater protection of Vero cells against ricin holotoxin compared to CC10501.

In one aspect, as described herein a new fluorescence polarization competition assay was developed to examine the structure-activity relationships (SAR) of a new series of compounds that bind at the P-stalk pocket of RTA and demonstrated that carboxylic acid is critical for activity. To determine if the different orientation of the thiophene carboxylate in RU-NT-93 is important for inhibition, planar compounds with different substituents were designed where the molecule is locked in a fixed conformation. Copounds RU-NT-59, PD00589, and RU-NT-206 were identified with the highest affinity and potency and solved the X-ray crystal structures of each compound independently bound to RTA by co-crystallization. The structure of RTA in complex with each inhibitor revealed that all three compounds bind similarly to the P-stalk binding pocket of RTA as CC10501 but reach deeper into the pocket and establish more favorable contacts that define a new mode of interaction.

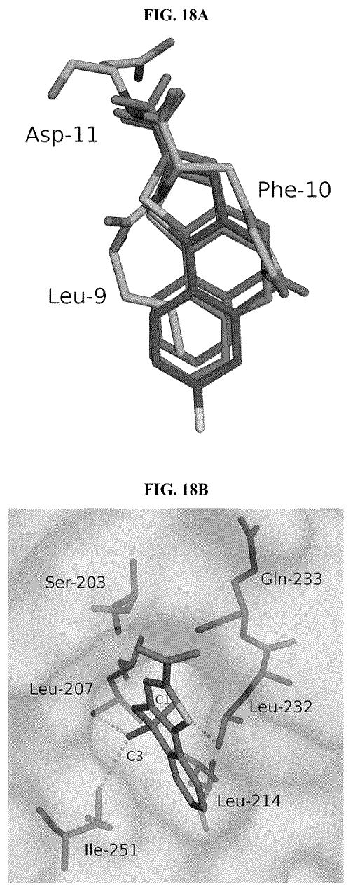

RU-NT-59 and RU-NT-206 showed higher binding affinity for RTA than the leading compound, RU-NT-93, and more potent IC₅₀ (Table 3), indicating that these inhibitors, especially RU-NT-206, bind residues in the P-stalk pocket in a way that results in more effective inhibition of RTA. The P-stalk binding pocket is quite distant from the active site of RTA at ~20 Å away (FIGS. 9A-9C), suggesting that the inhibitory effect of these compounds is due to the preclusion of RTA interaction with the P-stalk. The crystal structures showed that RU-NT-59 and PD00589 bind the P-stalk pocket and form most of the same contacts as RU-NT-93, including hydrophobic connections with Tyr183, Phe240, and a salt-bridge with the side chain of Arg235 (FIGS. 11A-11B). The cyclohexene and thiophene rings in RU-NT-59 and PD00589 occupy the same pocket space as the P11 residues Phe10 and Asp11, respectively, effectively competing with these P-stalk residues for binding to RTA as is reflected in the fluorescence polarization experiments (FIGS. 6A-6F). The benzene rings from RU-NT-59 and PD00589 overlap with P11's Leu9 upon superposition (FIG. 18A), and hydrophobically interact with RTA residues I1e247, Leu248, and I1e251 (FIG. 11A-11B). The fluoride atom within the fluorobenzene ring of RU-NT-59 contacts Val242 in RTA and forms much closer contacts with I1e247 and Leu248 (FIG. 11C) relative to PD00589, conceivably resulting in the slightly higher affinity of RU-NT-59.

RU-NT-206 binds the P-stalk pocket forming most of the same contacts as RU-NT-59 and PD00589 including hydrophobic connections with Tyr183, Phe240, I1e247, I1e251, and a stronger salt-bridge with the side chain of Arg235 (FIGS. 11A-11B and FIG. 11D). The similar pose of RU-NT-206 to RU-NT-59 and PD00589 within the P-stalk pocket equivalently placed the cyclohexene and thiophene rings in RU-NT-206 where they superposed onto P11 residues Phe10 and Asp11, respectively (FIG. 18A). The RU-NT-206 benzene ring likewise overlapped with P11's Leu9 upon superposition (FIG. 17C and FIG. 18A). RU-NT-206 had the highest binding affinity for RTA and considerably stronger IC₅₀ value compared to the other compounds in Table 2 either by measuring the displacement of the labeled P11 peptide from RTA by FP or by measuring the inhibition of depurination of rat liver ribosomes by RTA in vitro by qRT-PCR (Table 3). Part of the higher affinity of RU-NT-206 may be due to the near perpendicular arrangement of the carboxylate, which leads to stronger ionic interactions with arginines.

Although RU-NT-206 is bound similarly to the P-stalk pocket as RU-NT-59 and PD00589, it formed an additional salt-bridge with Arg234. RU-NT-206 established nonpolar contacts with Leu207, which lines a hydrophobic cavity within the P-stalk pocket along with Leu214, Leu232, and I1e251 (FIG. 18B). Consequently, the proximity of the methyl groups on RU-NT-206 to Ser203 and Gln233 within this mostly hydrophobic cavity may be responsible for more optimal affinity and higher potency. The added contacts of RU-NT-206 within the binding site conceivably contribute to the improved binding affinity of RU-NT-206 compared to RU-NT-59 and PD00589 giving RU-NT-206 the best capacity to block P-stalk binding of RTA in vitro and in cells.

The binding affinity of the three improved compounds correlated with the in vitro potency measured by two independent assays and potency in cell-based assays, indicating similar thermodynamic and biological functions. These results demonstrate that as the affinity for RTA is improved inhibitory potency in vitro and in cells is also improved, suggesting that compounds optimized for binding to the P-stalk pocket of RTA will more potently inhibit ricin. A lead compound RU-NT-206 (MW 258 Da) showed identical affinity and similar inhibitory potency against RTA as a five-fold larger P11 peptide (MW 1218 Da) and cell protection against Stx2a holotoxin for the first time.

TABLE 4
Fluorescence anisotropy (FA)-based competitive
assay data for certain compounds

| | Kᵢ | | IC₅₀ RTA | EC₅₀ |
|---|---|---|---|---|
| | FA^{a} | IC₅₀ FA^{b} | Rat Rib.^{c} | Ricin Vero^{d} |
| Compound | (μM) | (μM) | (μM) | (μM) |
| RU-NT-94 | 8 | 27 | | |
| RU-NT-116 | 12 | 42 | | |
| RU-NT-120 | 17 | 59 | | |
| RU-NT-124 | 5 | 21 | 23 | 47 |
| RU-NT-139 | 8 | 29 | | |
| RU-NT-159 | 8 | 30 | | |
| RU-NT-183 | 132 | 40 | | |
| RU-NT-186 | 19 | 64 | | |
| RU-NT-192 | 1 | 6 | 18.5 | 50 |
| RU-NT-202 | 4 | 15 | 31.5 | 169 |
| RU-NT-203 | >1000 | >1000 | | |
| RU-NT-206 | 0.6 | 4 | 18 | 29 |
| RU-NT-231 | >250 | >250 | | |
| RU-NT-245 | 5 | 20 | 2 | 68.5 |
| RU-NT-246 | 8 | 29 | | |
| RU-NT-247 | 22 | 75 | | |
| RU-NT-248 | 45 | 148 | | |
| RU-NT-249 | 23 | 77 | | |
| RU-NT-250 | 301 | 574 | | |
| RU-NT-252 | 130 | 498 | | |
| RU-NT-255 | 0.27 | 3 | 9.6 | 51 |
| RU-NT-274 | 1.8 | 9 ± 0.3 | 9 ± 2 | 93 ± 20 |
| RU-NT-277 | >64.5 | >250 | | |
| RU-NT-281 | 2.8 | 12.9 ± 0.8 | 3.6 | 87 |
| RU-NT-282 | >64.5 | >250 | | |
| RU-NT-283 | 48 | 160 | | |
| RU-NT-284 | >64.5 | >250 | | |
| RU-NT-285 | 22 | 86.8 | | |
| RU-NT-286 | 4.9 | 21 | | |
| RU-NT-287 | 41.4 | 161.3 | | |
| RU-NT-288 | 95.4 | 369 | | |
| RU-NT-289 | 74.1 | 287 | | |
| RU-NT-290 | 3.3 | 15 | | |
| RU-NT-291 | 68 | 263.4 | | |
| RU-NT-293 | 9.2 | 37.4 | | |
| RU-NT-294 | 2.1 | 10.3 | | 21 |
| RU-NT-295 | 11.5 | 46.4 | | |
| RU-NT-296 | >25.5 | >100 | | |
| RU-NT-297 | 3.4 | 15.4 | | |
| RU-NT-298 | >25.5 | >100 | | |
| RU-NT-299 | >25.5 | >100 | | |
| RU-NT-300 | >25.5 | >100 | | |
| RU-NT-301 | 4.3 | 18.6 | | |
| RU-NT-302 | 1.8 | 9.1 | 68 | 80 |
| RU-NT-303 | 7.6 | 31.2 | | |
| RU-NT-304 | 6.7 | 28 | | |
| RU-NT-305 | 3.3 | 14.9 | | |
| RU-NT-306 | >25.5 | >100 | | |
| RU-NT-307 | >64.5 | >250 | | |
| RU-NT-308 | 0.86 | 5.5 | U.D.^{e} | 32 (n = 3.1) |
| RU-NT-311 | >25.5 | >100 | | |
| RU-NT-312 | >25.5 | >100 | | |
| RU-NT-313 | >25.5 | >100 | | |
| RU-NT-316 | 0.7 | 5 | 38 | |
| RU-NT-317 | >25.5 | >100 | | |
| RU-NT-318 | 22 | 86.8 | | |
| RU-NT-319 | 9.5 | 38.5 | | |
| RU-NT-320 | 11.1 | 45 | | |
| RU-NT-321 | 14.9 | 59.5 | | |
| RU-NT-323 | 0.5 | 4 ± 0.2 | 9 ± 1 | 17 ± 1 |
| RU-NT-324 | >25.5 | >100 | | |
| RU-NT-325 | >25.5 | >100 | | |
| RU-NT-326 | 2.5 | 11.8 | | |
| RU-NT-327 | >25.5 | >100 | | |
| RU-NT-328 | 2 | 10 | | |
| RU-NT-330 | 6 | 22 | | |
| RU-NT-331 | >100 | >1000 | | |
| RU-NT-339 | >100 | | | |
| RU-NT-340 | >100 | | | |
| RU-NT-341 | >100 | >100 | | |
| RU-NT-342 | >100 | | | |
| RU-NT-343 | >100 | | | |
| RU-NT-344 | 2.5 | 11.7 | | |
| RU-NT-345 | >100 | | | |
| RU-NT-349 | >100 | >1000 | | |
| RU-NT-350 | >100 | >1000 | | |
| RU-NT-360 | 2.8 | 13.1 | | |
| RU-NT-361 | 8 | 33 ± 2 | | |
| RU-NT-363 | 3.2 | 14.3 ± 0.9 | 89.8 | |
| RU-NT-366 | 3 | 13.6 ± 1.6 | 75 | |
| RU-NT-373 | 2 | 10 ± 1 | 27 ± 6 | 13 ± 2 |
| RU-NT-382 | 5 | 21.3 | | |
| RU-NT-384 | 2.1 | 10.1 | | |
| RU-NT-387 | 2.6 | 12 | | |
| RU-NT-389 | 7.8 | 32 | | |
| RU-NT-391 | 1.1 | 6.3 | 80 | |
| RU-NT-392 | 1.8 | 9.2 | 15.2 | |
| RU-NT-395 | 6.4 | 26.9 | | |
| RU-NT-399 | 2.5 | 11.7 | | |
| RU-NT-401 | 2.6 | 12 | | |
| RU-NT-403 | 1 | 6 ± 0.2 | 8 ± 2 | 83 ± 23 |
| RU-NT-417 | 2.7 | 12.5 | | |
| RU-NT-419 | 2.1 | 10.4 | | |
| RU-NT-421 | 1.6 | 8 ± 0.3 | 25 ± 20 | 35 ± 3 |
| RU-NT-422 | 0.7 | 5 ± 0.2 | 20 ± 1 | 23 ± 2 |
| RU-NT-423 | 2.7 | 12.7 | | |
| RU-NT-424 | 2 | 9.8 | 44 | |
| RU-NT-430 | 1.8 | 9 ± 0.7 | 35 ± 2 | 20.2 ± 2 |
| RU-NT-438 | 3.4 | 15.1 | | |
| RU-NT-439 | 2.5 | 11.9 | 33 | 120.2 |
| RU-NT-444 | 9.9 | 40.1 | | |
| RU-NT-446 | 2.4 | 11.2 | 24 | |
| RU-NT-447 | 3.5 | 15.6 | | |
| RU-NT-449 | 0.5 | 4 ± 0.1 | 15 ± 3 | >500 |
| RU-NT-450 | 3.3 | 14.7 | | |

^{a}The Kᵢ values were measured by fluorescence anisotropy (FA) using BioTek Synergy H1 plate reader; ^{b}The FA IC₅₀ value represents the displacement of the fluorescent P11 probe from RTA; ^{c}The qRT-PCR IC₅₀ value represents inhibition of RTA-mediated depurination of rat liver ribosomes in vitro; ^{d}The EC₅₀ value is the half-maximal concentration required to inhibit depurination by ricin holotoxin in Vero cells as determined by qRT-PCR.
^{e}U.D., unable to determine.

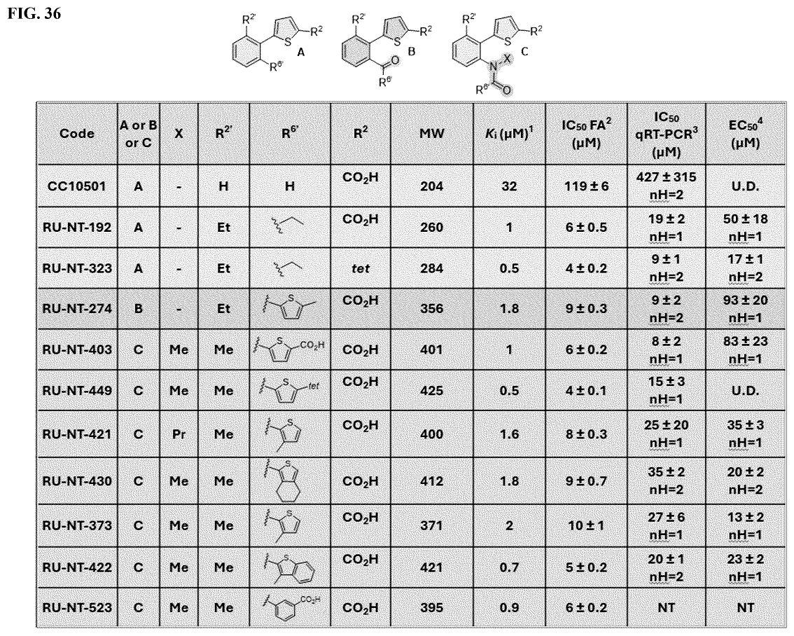

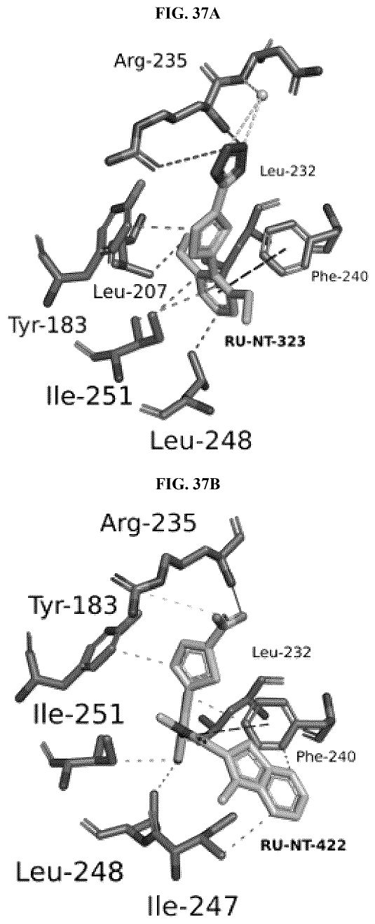

RU-NT-192 was initially identified as a lead compound targeting ricin. Guided by structure-based design and computational modeling, this scaffold was systematically optimized, and the resulting analogs were evaluated using both biochemical binding assays and cell-based potency studies. An overview of the medicinal chemistry optimization of RU-NT-192 is presented in FIG. 35, while the chemical structures of 249 synthesized compounds and their corresponding protection data are shown in Table 4.

Binding affinities and half-maximal inhibitory concentrations (IC₅₀) were determined using a fluorescence anisotropy (FA)-based competitive binding assay. This assay quantifies the decrease in FA resulting from displacement of a fluorescently labeled P11 peptide, which binds to the P-stalk site of RTA. The FA_IC₅₀ value represents the inhibitor concentration required to displace 50% of the P11 probe from RTA. Inhibition constants (Ki) were calculated from the IC₅₀ values and represent the concentration of inhibitor required to occupy half of the available RTA binding sites at equilibrium.

All compounds were initially screened by measuring percent inhibition in the FA assay at three concentrations (10 μM, 50 μM, and 250 μM). Compounds exhibiting ≥40% inhibition at 10 μM were subjected to full titration analyses to determine IC₅₀ and Ki values. For compounds with FA_IC₅₀ values below 10 μM, in vitro functional activity was further assessed by measuring inhibition of RTA-mediated depurination of rat liver ribosomes using a qRT-PCR assay. Depurination data were fit using the Hill equation, where n in Table 4 represents the Hill coefficient. The depurination IC₅₀ (Dep_IC50) is defined as the inhibitor concentration required to reduce RTA-catalyzed ribosome depurination by 50% in vitro.

Cell-based efficacy was evaluated by examining the ability of selected compounds to inhibit ricin holotoxin-mediated depurination and cytotoxicity in Vero cells. In the presence of 200 μM ricin holotoxin, ribosome depurination increased linearly over a 4-hour period. Two hours after the simultaneous addition of ricin holotoxin and inhibitor, total cellular RNA was isolated and analyzed by qRT-PCR to quantify depurination levels. Ribosome depurination was assessed across multiple inhibitor concentrations to establish dose-response relationships, from which half-maximal effective concentrations (Vero-EC₅₀) were determined when applicable. Protection data for the most active compounds identified in this screen are presented in FIG. 36.

To further improve binding affinity, replacements for the carboxylic acid moiety of RU-NT-192 (Ki=1 μM) were examined. Neutral hydrogen-bonding groups designed to interact with Arg235, including amide (RU-NT-324, Ki>100 μM), ketone (RU-NT-341, Ki>100 μM), and N-hydroxyamidine (RU-NT-325, Ki>25.5 μM), were inactive, underscoring the critical importance of an acidic functionality at this position. Several acidic isosteres were also explored. Hydroxamic acid (RU-NT-289, pKa=8.47, Ki=74 μM), reverse methylated acylsulfonamide (RU-NT-327, Ki>25.5 μM), and substituted hydroxypyrazoles (RU-NT-339, RU-NT-340, RU-NT-342; Ki>100 μM) all showed poor activity. Slightly acidic analogs, such as methoxyhydroxamic acid (RU-NT-290, pKa=10.9), retained activity (Ki=3.3 μM) but did not improve upon the parent compound. Similarly, acylsulfonamide (RU-NT-363, Ki=3 μM) and oxadiazolinone (RU-NT-326, Ki=2.5 μM) afforded only modest gains. RU-NT-363 exhibited a depurination IC₅₀ of 90 μM and failed to produce a measurable dose response in Vero cells.

In contrast, the tetrazole analog RU-NT-323 (Ki=0.5 μM) demonstrated a twofold improvement in binding affinity relative to RU-NT-192. RU-NT-323 inhibited RTA-mediated depurination of rat liver ribosomes with an IC₅₀ of 9±1 μM and protected Vero cells from ricin holotoxin-induced depurination with an EC₅₀ of 17±1 μM. Overall, RU-NT-323 exhibited approximately 2-fold improvements in both binding affinity and in vitro depurination potency relative to RU-NT-192, likely due to enhanced electrostatic interactions with Arg235 and/or deeper penetration into the hydrophobic pocket. Notably, Vero cell potency improved by approximately 3-fold over RU-NT-192. RU-NT-323 also conferred improved protection against ribosome depurination by Shiga toxin 2 (Stx2A1), with an IC₅₀ of 47±9 μM compared to 99±25 μM for RU-NT-192.

Building on the non-coplanar orientation observed in the X-ray structure of RU-NT-192 (Ki=1 μM), fragment-growth strategies were pursued using the simpler 2-methyl analog to access additional pockets within the P-stalk binding site and improve RTA affinity. Substitution at the 6-position with phenylmethylene (RU-NT-302, Ki=1.8 μM), extension to an ethylene linker (RU-NT-305, Kᵢ=3.3 μM), introduction of a double bond (RU-NT-304, Ki=6.7 μM), or a phenylacetylene moiety (RU-NT-303, Ki=7.6 μM) uniformly reduced binding affinity. Replacing the phenyl group in RU-NT-302 with cis-ethylene 5-methylthiophene resulted in loss of activity (RU-NT-331, Ki>100 μM). In contrast, the corresponding trans isomer (RU-NT-330, Ki=6 μM) retained moderate activity, consistent with molecular modeling (MOE, SeeSAR) suggesting favorable thiophene positioning for hydrogen bonding with Arg234.

To further enforce non-coplanarity, a carbonyl group was introduced (RU-NT-274, Ki=1.8 μM). Molecular modeling predicted optimal interactions, including hydrogen bonding between the thiophene sulfur and carbonyl oxygen with Arg234, as well as H-π/π-π stacking interactions with Phe240. Reduction of the ketone and carboxylic acid to the corresponding alcohol (RU-NT-282) or esterification to a methyl ester (RU-NT-277) reduced affinity, highlighting the importance of carbonyl functionality. Removal of the ketone altogether (RU-NT-281, Ki=2.8 μM) resulted in an approximately 1.5-fold loss of potency relative to RU-NT-274. Further optimization with a benzothiophene scaffold bearing an ortho-cyclopropyl substituent (RU-NT-308, Ki=0.9 μM) afforded a two-fold improvement over RU-NT-274, likely due to enhanced hydrophobic contacts and 7r-stacking interactions with Phe240. In cellular assays, RU-NT-308 showed a ~3-fold improvement in Vero cell protection against ricin holotoxin (EC₅₀=32 μM) relative to RU-NT-274 (EC₅₀=93 μM) and RU-NT-281 (EC₅₀=87 μM). Collectively, these data underscore the importance of di-ortho substitution and enforced non-coplanarity for achieving high-affinity RTA inhibition.

To further enhance RTA affinity while improving drug-like properties, isosteric replacement of the ketone in RU-NT-274 and RU-NT-308 with a methylated amide was explored. This strategy was intended to improve metabolic stability, reduce electrophilic reactivity, and introduce additional hydrogen-bonding capacity, while tuning lipophilicity and solubility. The initial amide analog (RU-NT-344, Ki=2.5 μM) showed reduced potency relative to RU-NT-274 (Ki=1.8 μM). Extending the methyl substituent on the phenyl ring to an ethyl group (RU-NT-395, Ki=6.4 μM) led to an additional three-fold loss in affinity, suggesting that the hydrophobic pocket favors a methyl substituent when paired with the two-atom amide spacer rather than the one-atom carbonyl spacer. Introduction of an ethylene spacer (RU-NT-394) resulted in a loss of activity.

Further optimization of the amide series focused on N-substitution, isosteric replacements, spacer length, phenyl ring modifications, carboxylic acid replacements, and thiophene substitutions. RU-NT-344 (Ki=2.5 μM) served as a lead compound for this series. The corresponding free amide (RU-NT-343, Ki>100 μM) was inactive, consistent with X-ray and modeling data indicating loss of the non-coplanar conformation between the phenyl and thiophene rings. Linear N-substituents, ethyl (RU-NT-387, Ki=2.6 μM) and butyl (RU-NT-377, 20% inhibition at 10 μM), provided little benefit, while improvement was observed for the propyl analog (RU-NT-392, Kᵢ=1.8 μM, RU-NT-421, Kᵢ=1.6 μM) analog. RU-NT-421 showed 1.4-fold higher protection in Vero cells compared to RU-NT-192. Branched or cyclic substituents (cyanomethylene, RU-NT-431; cyclobutyl, RU-NT-393; cyclopropyl, RU-NT-435) further reduced affinity, likely by perturbing the torsional angle required for optimal positioning of the phenyl ring within the hydrophobic pocket defined by Leu207, Leu232, and I1e251. Overall, the methyl substituent emerged as optimal, balancing non-coplanarity, molecular weight, and conformational control within the P-stalk binding site.

Spacer length and amide isosteres were also evaluated. Extension of the spacer by one or two methylene units (RU-NT-368, RU-NT-345) relative to the parent analogs (RU-NT-344, RU-NT-343) rendered the compounds inactive, likely due to increased conformational entropy and unfavorable desolvation associated with added flexibility. A reverse amide designed to restore non-coplanarity by replacing the N-methyl group with a ketone (RU-NT-432) was inactive compared to RU-NT-384 (Ki=2.1 μM). Similarly, a methylated sulfonamide isostere (RU-NT-484, Ki=4.6 μM) exhibited a 3-fold reduction in affinity relative to its amide counterpart (RU-NT-373, Ki=2.0 μM). RU-NT-373 inhibited in vitro depurination with an IC₅₀ of 27+6 μM and protected Vero cells from ricin holotoxin with an approximately 4-fold improvement in EC₅₀ (13+2 μM) compared to RU-NT-192 (FIG. 36). Urea-based analogs intended to enhance polarity and metabolic stability were also poorly tolerated: the dimethyl urea (RU-NT-385) was inactive, while fused ethylene (RU-NT-396, 21% inhibition at 10 μM) and propylene (RU-NT-397, inactive) variants showed markedly reduced activity relative to RU-NT-344 (Ki=2.5 μM). Taken together, these results highlight the stringent structural requirements of the P-stalk binding site and confirm the original amide configuration as optimal for maintaining binding affinity and conformational integrity.

Further optimization focused on systematic modification of the thiophene substituent by varying the group at the 5-position of RU-NT-344. Replacement of the methyl group with small lipophilic substituents, including ethyl (RU-NT-399) and fluoro (RU-NT-416), did not improve binding affinity relative to RU-NT-344. Introduction of polar functionalities at this position was pursued to enhance interactions with Arg234. These included acetyl (RU-NT-450, Kᵢ=3.3 μM), amide (RU-NT-447, Kᵢ=3.5 μM), methoxy (RU-NT-401, Kᵢ=2.6 μM), dioxine (RU-NT-423, Kᵢ=2.7 μM), dihydropyran (RU-NT-418, inactive), carboxylic acid (RU-NT-403, Kᵢ=1.0 μM), and tetrazole (RU-NT-449, Kᵢ=0.5 μM). With the exception of the ionizable carboxylic acid and tetrazole, these substitutions produced either no improvement or a slight reduction in binding affinity. The carboxylic acid and tetrazole analogs demonstrated ~3.5-fold and ~5.5-fold improvements in binding affinity, respectively, attributable to favorable ionic interactions with the positively charged Arg234, as supported by molecular modeling using MOE and SeeSAR.

RU-NT-403 also exhibited a 2.3-fold improvement in the in vitro depurination IC₅₀ (6±0.2 μM) relative to RU-NT-192 (FIG. 36). However, both RU-NT-403 and RU-NT-449 failed to surpass RU-NT-192 in Vero cell assays, likely due to reduced cell permeability associated with increased acidity. Consistent with this interpretation, the dimethyl ester of RU-NT-403 (RU-NT-452) and the methyl ester of RU-NT-449 (RU-NT-448) were inactive in the FA assay, underscoring the critical role of acidity in target engagement. An acyl sulfonamide isostere (RU-NT-514; 34% FA inhibition at 10 μM) showed lower affinity than the corresponding carboxylic acid and tetrazole analogs.

Bulky hydrophobic substituents at the 5-position were also evaluated, including trifluoromethyl (RU-NT-451; 30% FA inhibition at 10 μM), tert-butyl (RU-NT-405; 2% FA inhibition at 10 μM), a fused cyclohexyl ring (RU-NT-430, Kᵢ=1.8 μM), and a fused phenyl ring (RU-NT-360, Kᵢ=2.8 μM). Most of these modifications resulted in weaker binding relative to RU-NT-344 (Kᵢ=2.5 μM), with the exception of RU-NT-430, which showed a modest ~1.5-fold improvement. RU-NT-430 also demonstrated a 2.5-fold improvement in cellular potency in Vero cells (EC₅₀=20+2 μM) compared to RU-NT-192 (FIG. 36), likely due to enhanced 71-71 stacking interactions with Phe240.

A methyl walk on the thiophene ring revealed that shifting the methyl substituent from the 5- to the 3-position (RU-NT-373, Kᵢ=2.0 μM) improved binding affinity, whereas substitution at the 4-position (RU-NT-376; 15% FA inhibition at 10 μM) did not. The improved activity at the 3-position is attributed to additional hydrophobic interactions with I1e247, as supported by MOE docking and corroborated by X-ray crystallography of RU-NT-422. In contrast, an unsubstituted thiophene (RU-NT-385) was inactive, confirming the importance of alkyl substitution at this position. Thiophene isosteres, including thiazole (RU-NT-380, RU-NT-382; Kᵢ=5 μM), furan (RU-NT-371), and isoxazole (RU-NT-369), did not improve binding affinity.

Further SAR exploration at the 3-position of the thiophene in RU-NT-373 included small substituents (nitrile: RU-NT-454, Kᵢ=3.2 μM; fluoro: RU-NT-456, inactive; methoxy: RU-NT-461, inactive; ethoxy: RU-NT-464, 24% FA inhibition at 10 μM), acidic groups (tetrazole: RU-NT-467), and bulky electron-withdrawing groups (trifluoromethyl: RU-NT-474). All resulted in reduced affinity or inactivity relative to the methyl substituent. Modifications to the phenyl ring, including additional methyl substitution (RU-NT-457) or replacement with a 5-pyridyl group (RU-NT-491), abolished activity, likely due to steric clashes and poor tolerance for polar functionalities within the hydrophobic P-stalk binding site.

To replicate the enhanced binding observed with benzothiophene analogs, a fused benzene ring was introduced to the thiophene core of RU-NT-373. The resulting 3-methyl benzothiophene analog RU-NT-422 (Kᵢ=0.7 μM) exhibited a ~3-fold improvement in binding affinity (FIG. 36), driven by additional H-7c interactions with Phe240 and hydrophobic contacts with I1e247, as confirmed by X-ray crystallography. RU-NT-422 also demonstrated a 2-fold improvement in cellular potency against ricin holotoxin in Vero cells (EC₅₀=20+1 μM) relative to RU-NT-192 (FIG. 36). Increasing the size of the 3-substituent (ethyl: RU-NT-470; cyclopropyl: RU-NT-472) reduced affinity, consistent with steric clashes at this position.

A methyl walk on the benzothiophene scaffold showed that substitution at positions 4 (RU-NT-417, Ki=2.7 μM) or 5 (RU-NT-424, Ki=2 μM) resulted in 3-4-fold reductions in affinity relative to RU-NT-422. Introduction of a methoxy group at position 5 (RU-NT-503, Ki=1.1 μM) slightly reduced potency, while additional methyl substitution on the phenyl ring (RU-NT-512, FA 20% inhibition at 10 μM) was detrimental due to steric constraints within the hydrophobic pocket. Substitution at position 4 with an olefin (RU-NT-516, Ki=2.3 μM) caused a modest loss of affinity.

Heteroatom substitution on the benzothiophene revealed that incorporation of a nitrogen at position 7 (RU-NT-478, Kᵢ=1.8 μM) or a fluoro substituent (RU-NT-486, Kᵢ=1.5 μM) improved binding relative to RU-NT-360 (Kᵢ=2.8 μM), likely through enhanced hydrogen-bonding interactions with Arg234. Fluoro (4-fluoro: RU-NT-499, Kᵢ=2.8 μM, 5-fluoro: RU-NT-469, Ki=2.4 μM, 6-fluoro: RU-NT-502, Ki=2.4 μM) and chloro walks (4-chloro: RU-NT-501, 5-chloro: RU-NT-471, 7-chloro: RU-NT-500) confirmed the 7-position as optimal, with 7-fluoro outperforming 7-chloro analogs. Positively charged pyridinium analogs (RU-NT-479, RU-NT-482, RU-NT-492) were inactive, consistent with electrostatic repulsion from nearby Arg234 and poor compatibility with the hydrophobic binding site. Small polar substituents at the 7-position, hydroxyl group (RU-NT-509, Ki=1.8 μM) or a methoxy group (RU-NT-506, Ki=2.0 μM) improved affinity relative to RU-NT-360 (Ki=2.8 μM).

Non-fused benzothiophene analogs were examined. An unsubstituted phenyl ring (RU-NT-490, Kᵢ=2.3 μM) modestly improved affinity relative to RU-NT-360. A 6-fluoro substitution (RU-NT-496, Kᵢ=1.5 μM) provided minor gains, whereas bulkier groups such as CF₃ (RU-NT-507) led to solubility issues and reduced activity. Combined substitution on the thiophene and phenyl rings (RU-NT-518) similarly resulted in reduced potency, driven primarily by poor solubility in biological assays.

Finally, replacement of the benzothiophene with a naphthalene bioisostere (RU-NT-515, Kᵢ=1.4 μM) reduced sulfur content while preserving hydrophobic interactions, resulting in a two-fold improvement relative to the unsubstituted benzothiophene (RU-NT-360, Kᵢ=2.8 μM). In contrast, simple phenyl replacements (RU-NT-517, RU-NT-520) led to reduced activity. The fully saturated cyclohexyl analog (RU-NT-519, Kᵢ=2.0 μM) provided only minor improvement, likely due to increased hydrophobic bulk without optimal aromatic interactions. Introduction of a carboxylic acid at the meta position (RU-NT-523, Kᵢ=0.9 μM) significantly enhanced affinity, whereas ortho substitution was less effective, consistent with modeling predictions indicating optimal positioning relative to Arg234.

X-ray crystal structures of RTA bound to RU-NT-323 and RU-NT-422 revealed that both inhibitors occupy the P-stalk binding pocket in closely related poses, with subtle yet functionally important differences. Both compounds engage a conserved hydrophobic cluster comprising Tyr183, Leu207, Leu232, Leu248, I1e251, and Phe240. RU-NT-422 further extends this network through additional contacts between its benzothiophene ring and I1e247 and Phe240. These hydrophobic anchors strongly influence ligand alignment relative to Arg234 and Arg235, residues central to P-stalk recognition and inhibitor potency. In both complexes, the inhibitors form specific hydrogen-bonding and salt-bridge interactions with Arg235, while their aromatic systems engage the same hydrophobic cluster and participate in H-7L/n-7L stacking interactions with Phe240.

The newly solved structures refine these interactions in complementary ways. In RU-NT-323, the ethyl substituent on the benzene ring penetrates deeper into a subpocket lined by Leu207, Leu232, and Gln233, enhancing hydrophobic complementarity and potentially strengthening overall affinity. In RU-NT-422, the benzothiophene ring establishes previously unobserved contacts with I1e247 and Phe240 and approaches Arg234 to within 4.1 Å, highlighting a clear opportunity to directly engage Arg234 in future designs. Collectively, these results indicate that enforced non-coplanarity and precise carbonyl positioning are key determinants of improved potency. Taken together, these structural insights demonstrate how subtle changes in fragment orientation, hydrophobic anchoring, aromatic positioning, and acidic-group geometry govern inhibitor potency and provide a strong framework for the rational optimization of next-generation RTA inhibitors.

TABLE 5
Fluorescence anisotropy (FA) assay data
for certain exemplary compounds

| Compound | FA_10 uM (%) | FA_50 uM (%) | FA_250 uM (%) |
|---|---|---|---|
| RU-NT-342 | | | 79.6 |
| RU-NT-343 | | | 66.7 |
| RU-NT-344 | | | 94.0 |
| RU-NT-345 | | | 12.8 |
| RU-NT-358 | | | 76.2 |
| RU-NT-359 | | | 78.5 |
| RU-NT-360 | | | 96.6 |
| RU-NT-361 | | | 95.4 |
| RU-NT-362 | | | 70.8 |
| RU-NT-363 | | | 93.4 |
| RU-NT-366 | 24.3 | 76.7 | 94.0 |
| RU-NT-367 | −27.6 | −0.9 | 54.8 |
| RU-NT-342 | | | 79.6 |
| RU-NT-343 | | | 66.7 |
| RU-NT-344 | | | 94.0 |
| RU-NT-345 | | | 12.8 |
| RU-NT-358 | | | 76.2 |
| RU-NT-359 | | | 78.5 |
| RU-NT-360 | | | 96.6 |
| RU-NT-361 | | | 95.4 |
| RU-NT-362 | | | 70.8 |
| RU-NT-363 | | | 93.4 |
| RU-NT-366 | 24.3 | 76.7 | 94.0 |
| RU-NT-367 | −27.6 | −0.9 | 54.8 |
| RU-NT-368 | −5.2 | 51.8 | 85.2 |
| RU-NT-369 | −21.1 | 21.5 | 72.0 |
| RU-NT-370 | −32.7 | −20.1 | 16.2 |
| RU-NT-371 | −26.7 | 1.9 | 60.6 |
| RU-NT-372 | −21.6 | 9.8 | 63.3 |
| RU-NT-373 | 49.3 | 81.6 | 93.9 |
| RU-NT-374 | 9.0 | 27.6 | 61.5 |
| RU-NT-375 | −0.8 | 2.5 | 2.8 |
| RU-NT-376 | 15.1 | 33.1 | 65.0 |
| RU-NT-377 | 19.8 | 58.8 | 88.1 |
| RU-NT-378 | 25.7 | 58.0 | 85.3 |
| RU-NT-379 | 9.8 | 29.7 | 66.6 |
| RU-NT-380 | 4.8 | 14.3 | 42.3 |
| RU-NT-381 | 0.1 | 3.7 | 15.1 |
| RU-NT-382 | 30.1 | 67.6 | 87.2 |
| RU-NT-383 | 10.2 | 31.6 | 65.9 |
| RU-NT-384 | 41.9 | 78.0 | 93.0 |
| RU-NT-385 | 7.6 | 34.5 | 69.6 |
| RU-NT-386 | 10.7 | 37.6 | 79.5 |
| RU-NT-387 | 41.2 | 75.4 | 92.5 |
| RU-NT-388 | 14.5 | 43.6 | 77.3 |
| RU-NT-389 | 23.5 | 64.0 | 89.7 |
| RU-NT-390 | −1.8 | −0.1 | 3.2 |
| RU-NT-391 | 28.7 | 80.3 | 94.8 |
| RU-NT-392 | 48.0 | 82.0 | 93.0 |
| RU-NT-393 | 22.0 | 65.0 | 87.0 |
| RU-NT-394 | 17.0 | 48.0 | 83.0 |
| RU-NT-395 | 21.0 | 64.0 | 90.0 |
| RU-NT-396 | 21.0 | 46.8 | 78.8 |
| RU-NT-397 | 3.8 | 26.7 | 61.1 |
| RU-NT-398 | 4.4 | 22.2 | 58.1 |
| RU-NT-399 | 44.6 | 75.7 | 90.0 |
| RU-NT-400 | 6.2 | 28.6 | 65.4 |
| RU-NT-401 | 42.4 | 77.5 | 89.4 |
| RU-NT-402 | 11.6 | 40.4 | 73.7 |
| RU-NT-403 | 64.4 | 88.5 | 92.9 |
| RU-NT-404 | 10.4 | 33.2 | 65.3 |
| RU-NT-405 | 2.3 | 5.0 | 25.5 |
| RU-NT-406 | 6.2 | 25.3 | 58.5 |
| RU-NT-407 | 6.3 | 30.3 | 68.9 |
| RU-NT-408 | 6.9 | 23.5 | 57.6 |
| RU-NT-409 | 0.3 | 2.2 | 6.5 |
| RU-NT-410 | 5.0 | 15.9 | 48.1 |
| RU-NT-411 | −2.6 | 22.6 | 61.2 |
| RU-NT-412 | −1.0 | 23.2 | 66.6 |
| RU-NT-413 | 3.1 | 29.9 | 66.2 |
| RU-NT-414 | 1.6 | 26.0 | 62.1 |
| RU-NT-415 | 34.7 | 70.3 | 87.9 |
| RU-NT-416 | 35.6 | 70.9 | 92.6 |
| RU-NT-417 | 43.2 | 81.9 | 92.6 |
| RU-NT-418 | 1.3 | 12.4 | 39.7 |
| RU-NT-419 | 46.7 | 79.0 | 89.5 |
| RU-NT-420 | −2.0 | 15.5 | 52.9 |
| RU-NT-421 | 53.1 | 88.3 | 94.8 |
| RU-NT-422 | 70.0 | 91.5 | 95.7 |
| RU-NT-423 | 40.6 | 75.8 | 89.6 |
| RU-NT-424 | 55.4 | 87.2 | 97.2 |
| RU-NT-425 | 4.0 | 25.2 | 64.5 |
| RU-NT-426 | 7.3 | 30.2 | 66.0 |
| RU-NT-427 | 2.9 | 17.1 | 55.4 |
| RU-NT-428 | 2.8 | 9.3 | 18.7 |
| RU-NT-429 | −5.1 | 4.5 | 57.8 |
| RU-NT-430 | 58.4 | 86.7 | 93.8 |
| RU-NT-431 | 12.0 | 28.0 | 66.7 |
| RU-NT-432 | 2.4 | 1.9 | 9.4 |
| RU-NT-433 | −1.7 | 3.2 | 13.8 |
| RU-NT-434 | 13.5 | 39.9 | 74.9 |
| RU-NT-435 | 9.4 | 25.6 | 65.4 |
| RU-NT-436 | 5.2 | 16.1 | 44.3 |
| RU-NT-437 | 15.2 | 50.6 | 83.2 |
| RU-NT-438 | 40.7 | 74.8 | 91.9 |
| RU-NT-439 | 47.3 | 80.9 | 92.9 |
| RU-NT-440 | 0.4 | 9.1 | 28.9 |
| RU-NT-441 | 11.3 | 35.6 | 67.7 |
| RU-NT-442 | 0.0 | 4.1 | 8.5 |
| RU-NT-443 | 34.4 | 72.7 | 90.5 |
| RU-NT-444 | 16.3 | 53.9 | 83.1 |
| RU-NT-445 | −4.6 | 0.0 | −3.2 |
| RU-NT-446 | 43.0 | 80.9 | 91.7 |
| RU-NT-447 | 39.8 | 77.9 | 93.2 |
| RU-NT-448 | 1.4 | 12.5 | 39.6 |
| RU-NT-449 | 69.9 | 91.3 | 97.0 |
| RU-NT-450 | 41.4 | 78.1 | 93.0 |
| RU-NT-451 | 30.3 | 71.8 | 92.5 |
| RU-NT-452 | −2.1 | −0.9 | 0.6 |
| RU-NT-453 | 13.9 | 37.0 | 74.1 |
| RU-NT-454 | 40.0 | 76.5 | 92.8 |
| RU-NT-455 | 10.6 | 26.1 | 61.6 |
| RU-NT-456 | 12.3 | 35.5 | 73.1 |
| RU-NT-457 | 4.4 | 6.7 | 18.6 |
| RU-NT-458 | 8.9 | 17.2 | 43.6 |
| RU-NT-459 | 5.3 | 25.8 | 59.9 |
| RU-NT-460 | 5.4 | 10.9 | 35.4 |
| RU-NT-461 | 2.0 | 14.8 | 42.1 |
| RU-NT-462 | 23.0 | 55.4 | 84.1 |
| RU-NT-463 | 24.6 | 60.4 | 86.8 |
| RU-NT-464 | 23.8 | 59.9 | 89.2 |
| RU-NT-465 | 10.1 | 34.9 | 68.3 |
| RU-NT-466 | 2.0 | 2.9 | 1.7 |
| RU-NT-467 | 24.0 | 58.9 | 86.8 |
| RU-NT-468 | 4.9 | 8.9 | 18.5 |
| RU-NT-469 | 45.5 | 81.8 | 93.2 |
| RU-NT-470 | 12.8 | 34.6 | 74.5 |
| RU-NT-471 | 45.7 | 84.2 | 92.9 |
| RU-NT-472 | 17.8 | 53.4 | 84.9 |
| RU-NT-473 | 3.8 | 9.3 | 37.0 |
| RU-NT-474 | 6.5 | 18.9 | 54.0 |
| RU-NT-475 | 4.9 | 20.4 | 51.7 |
| RU-NT-476 | 46.8 | 82.2 | 93.2 |
| RU-NT-477 | 11.2 | 29.1 | 67.4 |
| RU-NT-478 | 50.0 | 81.9 | 94.0 |
| RU-NT-479 | −0.3 | 3.1 | 21.7 |
| RU-NT-480 | 6.9 | 26.0 | 64.5 |
| RU-NT-481 | 40.2 | 76.6 | 92.5 |
| RU-NT-482 | 2.3 | 15.5 | 42.6 |
| RU-NT-484 | 36.1 | 73.3 | 93.1 |
| RU-NT-486 | 57.5 | 86.6 | 94.2 |
| RU-NT-487 | −18.9 | −5.5 | 32.0 |
| RU-NT-488 | 73.2 | 91.4 | 96.9 |
| RU-NT-489 | −4.9 | 16.1 | 55.8 |
| RU-NT-490 | 51.3 | 85.3 | 95.1 |
| RU-NT-491 | −0.1 | 5.6 | 13.1 |
| RU-NT-492 | −0.7 | 29 | 57.7 |
| RU-NT-496 | 64.3 | 91.2 | 94.2 |
| RU-NT-499 | 46.7 | 83.9 | 95.7 |
| RU-NT-500 | 35.8 | 84.0 | 94.5 |
| RU-NT-501 | 37.8 | 80.3 | 94.5 |
| RU-NT-502 | 41.8 | 80.3 | 92.0 |
| RU-NT-503 | 67.2 | 92.5 | 95.6 |
| RU-NT-505 | 16.3 | 53.3 | 81.4 |
| RU-NT-506 | 51.9 | 85.8 | 94.2 |
| RU-NT-507 | 16.4 | 54.8 | 85.1 |
| RU-NT-508 | 51.5 | 86.3 | 94.0 |
| RU-NT-509 | 51.9 | 84.4 | 93.0 |
| RU-NT-510 | 2.9 | 2.9 | 0.1 |
| RU-NT-511 | 36.4 | 71 | 90.3 |
| RU-NT-512 | 19.9 | 63.6 | 90.2 |
| RU-NT-514 | 33.9 | 70 | 90.3 |
| RU-NT-515 | 61.1 | 90.7 | 96.6 |
| RU-NT-516 | 48.0 | 88.6 | 97.0 |
| RU-NT-517 | 18.5 | 59.6 | 84.9 |
| RU-NT-518 | 31.9 | 78.0 | 93.3 |
| RU-NT-519 | 43.6 | 80.0 | 92.5 |
| RU-NT-520 | 17.2 | 52.6 | 80.6 |
| RU-NT-521 | 12.5 | 48.1 | 80.8 |
| RU-NT-522 | −0.8 | 10.8 | 49.0 |
| RU-NT-523 | 60.4 | 87.6 | 95.0 |

TABLE 6
Concentration-dependent Vero cell assay data for exemplary compounds

| Compound | Percent Growth Inhibition at Compound Concentration | | | | | | |
|---|---|---|---|---|---|---|---|
| No. | 500 μM | 250 μM | 125 μM | 62.5 μM | 31.3 μM | 15.6 μM | 7.8 μM |
| RU-NT- | | 38.2 | 78.8/36.2 | 71/48.9 | 69.9/21.5 | 48.1 | 47.1 |
| 363 | | | | | | | |
| RU-NT- | 21.7 | 55.0/−99.4 | 54/−43.3 | 69.2 | 37.1 | 61 | 42.8 |
| 448 | | | | | | | |
| RU-NT- | 92.7 | 87.9 | 68.5 | | | | |
| 469 | | | | | | | |
| RU-NT- | 99.1 | 89.9 | 69.6 | | | | |
| 471 | | | | | | | |
| RU-NT- | 76.4 | 64.5 | 68.7 | | | | |
| 478 | | | | | | | |
| RU-NT- | 99.6 | 94.6 | 77.2 | | | | |
| 486 | | | | | | | |

### SEQUENCE LISTING

(P11)
SEQ ID NO: 1
SDDDMGFGLFD
SEQ ID NO: 2
GATGTCGGCTCTTCCTATCATTGT
SEQ ID NO: 3
CCAGCTCACGTTCCCTATTAGTC
SEQ ID NO: 4
TGCCATGGTAATCCTGCTCAGTA
SEQ ID NO: 5
TCTGAACCGCGGTTCCACA

### Enumerated Embodiments

The following exemplary embodiments are provided, the numbering of which is not to be construed as designating levels of importance:

Embodiment 1: A compound of Formula (I) or a pharmaceutically acceptable salt thereof:

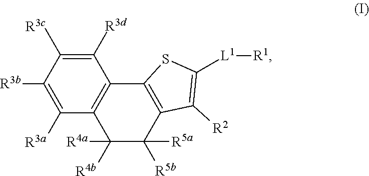

wherein:
   R¹ is a carboxylic acid (—COOH), —C(═O)C(OH)₂C(═O)OR^{A}, a carboxylic acid bioisostere, or a carboxylic acid precursor;
   R² is H;
   R^{3a}, R^{3b}, R^{3c}, and R^{3d} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C6-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   L¹ is selected from the group consisting of a bond and optionally substituted C₁-C₂ alkylenyl;
   R^{4a} and R^{4b} are each independently selected from the group consisting of H and optionally substituted C₁-C₆ alkyl,
      wherein no more than one of R^{4a} and R^{4b} is H;
   R^{5a} and R^{5b} are each independently selected from the group consisting of H and optionally substituted C₁-C₆ alkyl,
      or R^{5a} and R^{5b} can combine with the carbon atom to which they are bound to form a carbonyl moiety (C═O);
   R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
   wherein the compound is not 5,5-dimethyl-4,5-dihydronaphtho[1,2-b]thiophene-2-carboxylic acid.

Embodiment 2: The compound of Embodiment 1, wherein R¹ is selected from the group consisting of

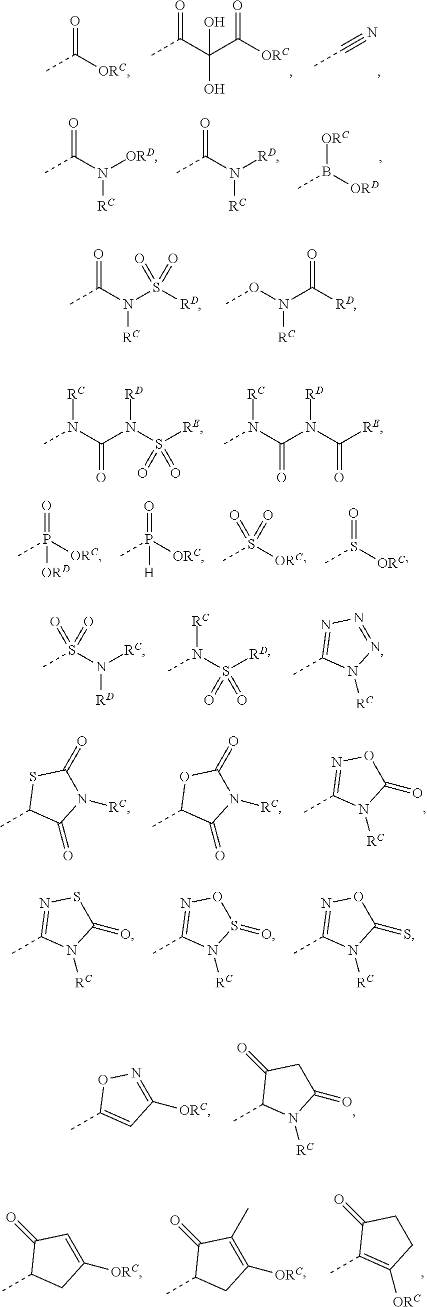

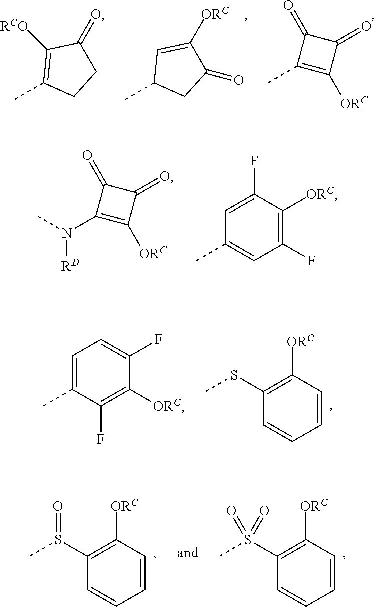

wherein R^{C}, R^{D}, and R^{E}, if present, are each independently selected from the group consisting of H, optionally substituted C₁-C₆ alkyl, and C(═O)(optionally substituted C₁-C₆ alkyl).

Embodiment 3. compound of Embodiment 1 or 2, wherein R¹ is selected from the group consisting of

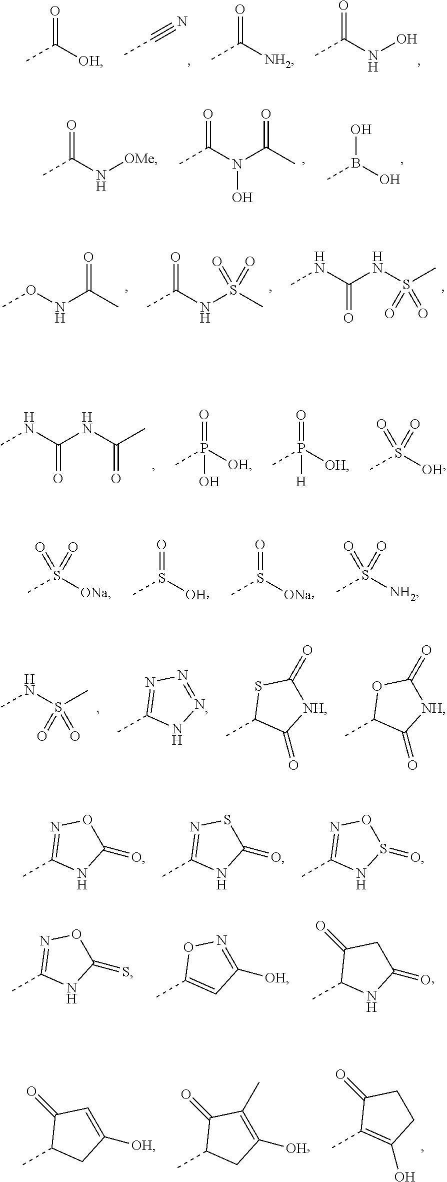

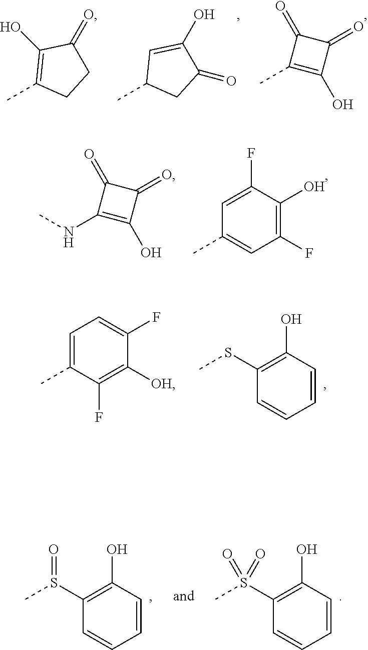

Embodiment 4: The compound of any one of Embodiments 1-3, wherein L¹ is a bond.

Embodiment 5: The compound of any one of Embodiments 1-4, wherein R^{4a} and R^{4b} are each independently CH₃.

Embodiment 6: The compound of any one of Embodiments 1-5, wherein R^{5a} and R^{5b} are each independently H.

Embodiment 7: The compound of any one of Embodiments 1-6, wherein R^{3a}, R^{3b}, R^{3c}, and R^{3d} are each independently selected from the group consisting of H, F, Cl, Br, CH₃, OCH₃, and N(CH₃)_{2.}

Embodiment 8: The compound of any one of Embodiments 1-7, wherein at least three of R^{3a}, R^{3b}, R^{3c}, and R^{3d} are H.

Embodiment 9: The compound of any one of Embodiments 1-8, wherein one of the following applies:
(a) R^{3b} is selected from the group consisting of F, Cl, Br, CH₃, OCH₃, and N(CH₃) and R^{3a}, R^{3c}, and R^{3d} are each H; and
(b) R^{3c} is selected from the group consisting of F, Cl, Br, CH₃, OCH₃, and N(CH₃) and R^{3a}, R^{3b}, and R^{3d} are each H.

Embodiment 10: The compound of any one of Embodiments 1-9, which is selected from the group consisting of:
5,5-dimethyl-4,5-dihydronaphtho[1,2-b]thiophene-2-carboxamide;
5,5-dimethyl-4,5-dihydronaphtho[1,2-b]thiophene-2-carbonitrile;
5-(5,5-dimethyl-4,5-dihydronaphtho[1,2-b]thiophen-2-yl)-2H-tetrazole;
5,5-dimethyl-N-(methylsulfonyl)-4,5-dihydronaphtho[1,2-b]thiophene-2-carboxamide
5,5-dimethyl-4-oxo-4,5-dihydronaphtho[1,2-b]thiophene-2-carboxylic acid; and methyl 3-(5,5-dimethyl-4,5-dihydronaphtho[1,2-b]thiophen-2-yl)-2,2-dihydroxy-3-oxopropanoate.

Embodiment 11: A compound of Formula (II) or a pharmaceutically acceptable salt thereof:

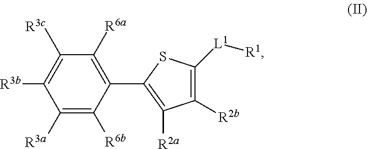

wherein:
   L¹ is selected from the group consisting of a bond and optionally substituted C₁-C₂ alkylenyl;
   R¹ is a carboxylic acid (—COOH), —C(═O)OR^{A}, —[C(═O)]₃R^{A}, —C(═O)C(OH)₂C(═O)OR^{A}, —C(═O)NHOR^{A}, H, a carboxylic acid bioisostere, or a carboxylic acid precursor;
   R^{2a} and R^{2b} are each independently H or optionally substituted C₁-C₆ alkyl;
   R^{3a}, R^{3b}, and R^{3c} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C6-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   R^{6a} and R^{6b} are each independently selected from the group consisting of H, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₆-C₁₀ aryl, optionally substituted C₂-C₈ heteroaryl, C(═O)R^{A}, and

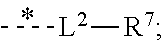

L² is selected from the group consisting of

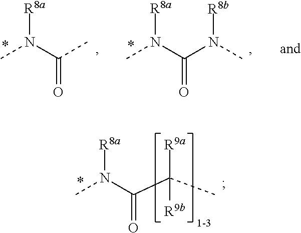

R⁷ is selected from the group consisting of

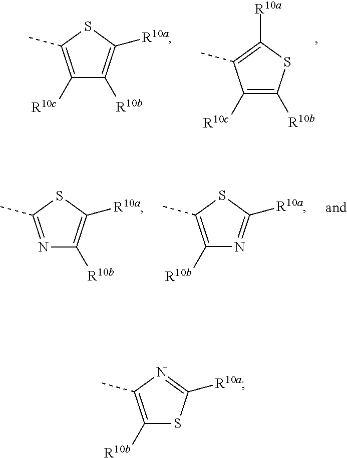

R^{8a} and R^{8b} are each independently selected from the group consisting of H, optionally substituted C₁-C₆ alkyl, and optionally substituted C₃-C₈ cycloalkyl, or R^{8a} and R^{8b} can combine with the atoms to which they are bound to form an optionally substituted C₂-C₈ heterocycloalkyl;
R^{9a} and R^{9b} are each independently selected from the group consisting of H and optionally substituted C₁-C₆ alkyl;
R^{10a}, R^{10b}, and R^{10c} are each independently selected from the group consisting of optionally substituted C₁-C₆ alkyl, optionally substituted C₃-C₈ cycloalkyl, optionally substituted C₆-C₁₀ aryl, optionally substituted C₆-C₈ heteroaryl, halogen, C(═O)R^{A}, C(═O)OR^{A}, C(═O)N(R^{A})(R^{B}), S(═O)R^{A}, S(═O)₂R^{A}, S(═O)N(R^{A})(R^{B}), and S(═O)₂N(R^{A})(R^{B}), or two vicinal substituents selected from the group consisting of R^{10a}, R^{10b}, and R^{10c} can combine with the atoms to which they are bound to form an optionally substituted C₃-C₈ cycloalkyl, optionally substituted C₂-C₈ heterocycloalkyl, optionally substituted C₆-C₁₀ aryl, or optionally substituted C₂-C₈ heteroaryl;
each occurrence of R^{A} and R^{B} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
wherein one of the following applies:
(a) R^{6a} and R^{6b} are each optionally substituted C₁-C₆ alkyl, wherein no more than one of R^{6a} and R^{6b} is optionally substituted C₁ alkyl;
(b) one of R^{6a} and R^{6b} is methyl, and one of R^{6a} and R^{6b} is selected from the group consisting of optionally substituted C₂-C₆ alkenyl, optionally substituted C₂-C₆ alkynyl, optionally substituted C₃-C₈ cycloalkyl, optionally substituted phenyl, and C₁-C₆ alkyl, wherein the optionally substituted alkyl is substituted with at least one optionally substituted phenyl;
(c) one of R^{6a} and R^{6b} is methyl, one of R^{6a} and R^{6b} is H, and one of R^{3a} and R^{3c} is selected from the group consisting of optionally substituted C₁-C₆ alkyl, optionally substituted C₂-C₆ alkenyl, optionally substituted C₂-C₆ alkynyl, optionally substituted C₃-C₈ cycloalkyl, optionally substituted phenyl, and C₁-C₆ alkyl;
(d) one of R^{6a} and R^{6b} is H, and one of R^{6a} and R^{6b} is selected from the group consisting of C₁-C₆ alkyl, optionally substituted C₁-C₆ haloalkoxy, optionally substituted C₂-C₆ alkenyl, optionally substituted C₂-C₆ alkynyl, and C(═O)H, wherein the C₁-C₆ alkyl is substituted with at least one substituent selected from the group consisting of C₂-C₈ alkynyl, C₁-C₆ alkoxy, C═O, and CN; and
(e) one of R^{6a} and R^{6b} is

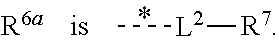

Embodiment 12: The compound of Embodiment 11, wherein R¹ is selected from the group consisting of

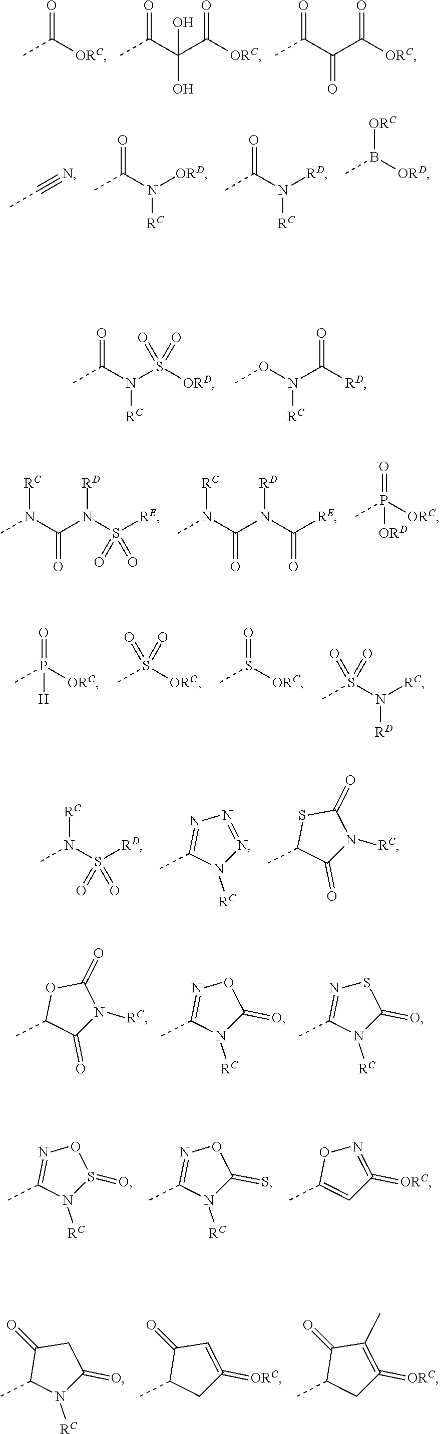

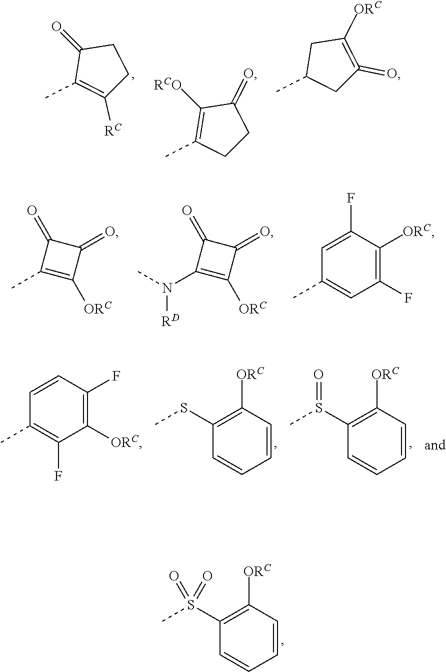

wherein R^{C}, R^{D}, and R^{E}, if present, are each independently selected from the group consisting of H, optionally substituted C₁-C₆ alkyl, and C(═O)(optionally substituted C₁-C₆ alkyl).

Embodiment 13: The compound of Embodiment 11 or 12, wherein R¹ is selected from the group consisting of

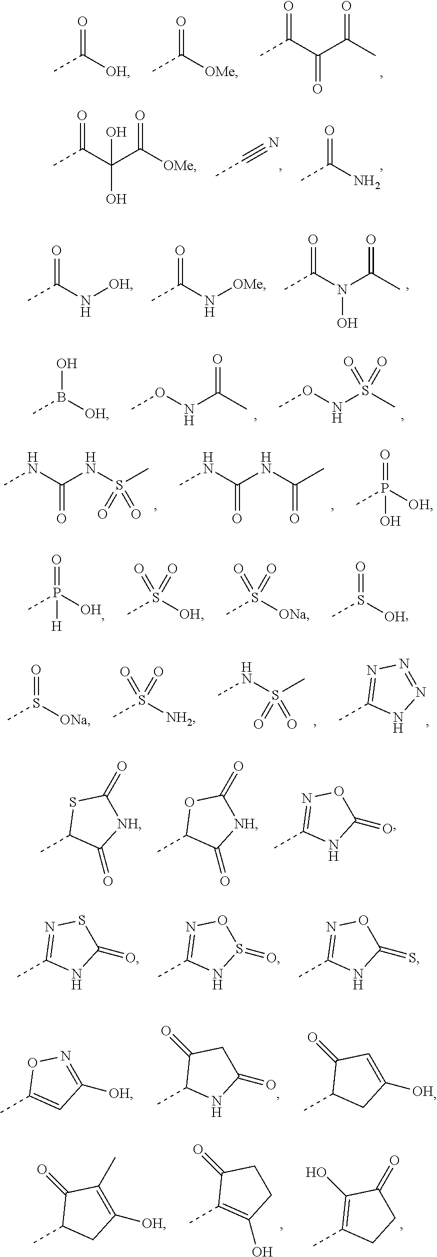

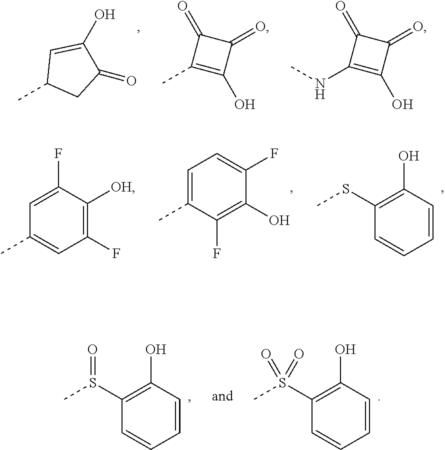

Embodiment 14: The compound of any one of Embodiments 11-13, wherein L¹ is a bond.

Embodiment 15: The compound of any one of Embodiments 11-14, wherein R^{3a}, R^{3b}, and R^{3c} are each independently selected from the group consisting of H, CH₃,

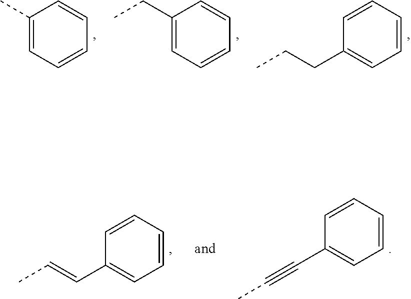

Embodiment 16: The compound of any one of Embodiments 11-15, wherein one of the following applies:
(a) R^{6a} is ethyl and R^{6b} is methyl;
(b) R^{6a} is ethyl and R^{6b} is ethyl; and
(c) R⁶ ais methyl and R^{6b} is ethyl.

Embodiment 17: The compound of any one of Embodiments 11-16, wherein

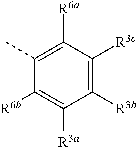

is selected from the group consisting of

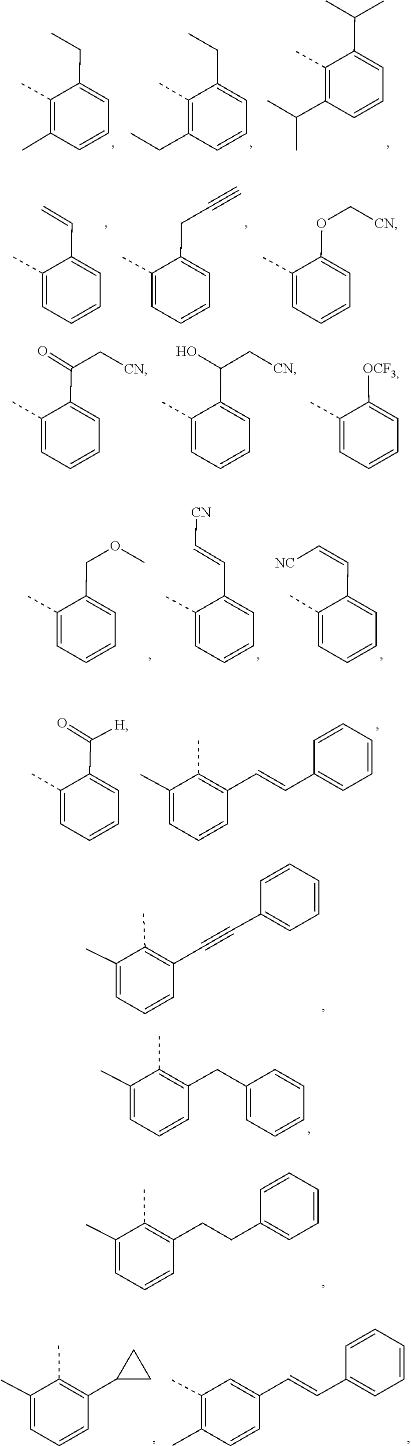

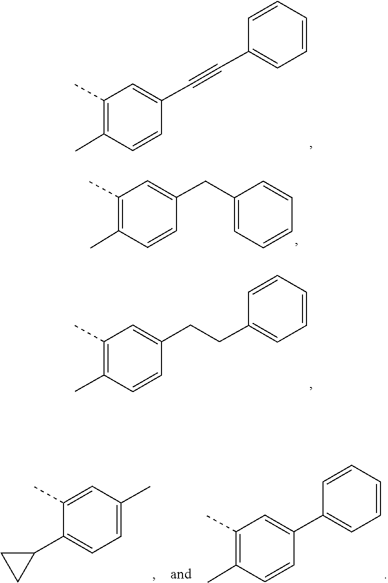

Embodiment 18: The compound of any one of Embodiments 11-14, wherein R^{6a} is

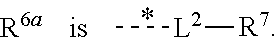

Embodiment 19: The compound of Embodiment 18, wherein R^{6b} is selected from the group consisting of H, CH₃, CF₃, and CH═CH₂.

Embodiment 20: The compound of Embodiment 18 or 19, wherein

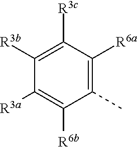

is selected from the group consisting of

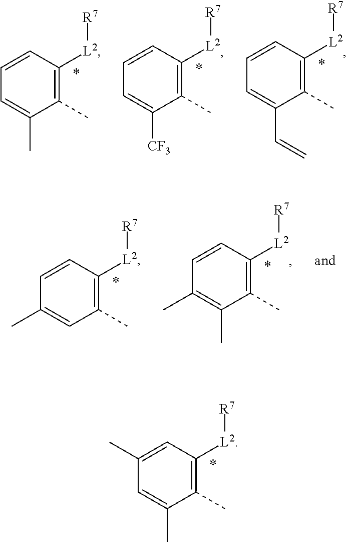

Embodiment 21: The compound of any one of Embodiments 18-20, wherein R^{8a} and R^{8b} are each independently selected from the group consisting of H, Me, Et, CH₂CN, CH₂C≡CH, iPr, nPr, nBu,

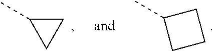

Embodiment 22: The compound of any one of Embodiments 18-20, wherein R^{8a} and R^{8b} combine with the atoms to which they are bound to form

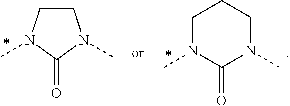

Embodiment 23: The compound of any one of Embodiments 18-22, wherein L² is selected from the group consisting of

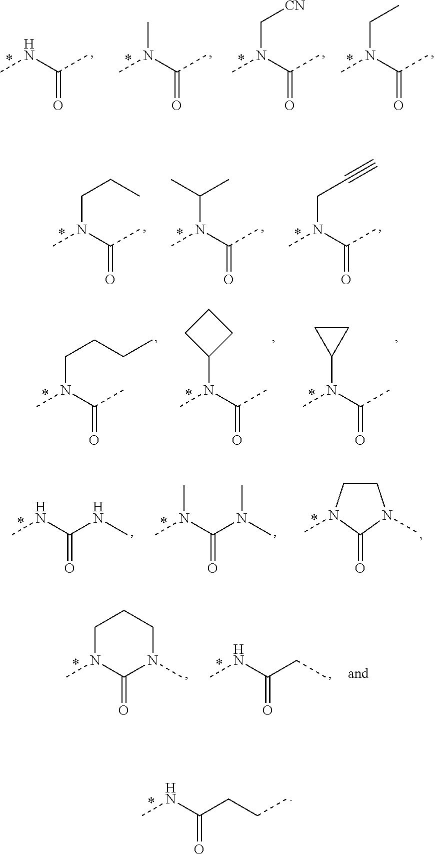

Embodiment 24: The compound of any one of Embodiments 11-23, wherein R^{10a}, R^{10b}, and R^{10c} are each independently selected from the group consisting of H, Me, CF₂H, CF₃, Et, tBu, F, C₁, OMe, CN, C(═O)OH, C(═O)OMe, C(═O)NH₂, C(═O)Me, S(═O)₂Me,

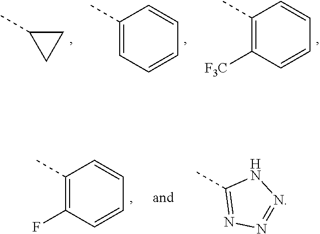

Embodiment 25: The compound of any one of Embodiments 11-24, wherein R⁷ is selected from the group consisting of

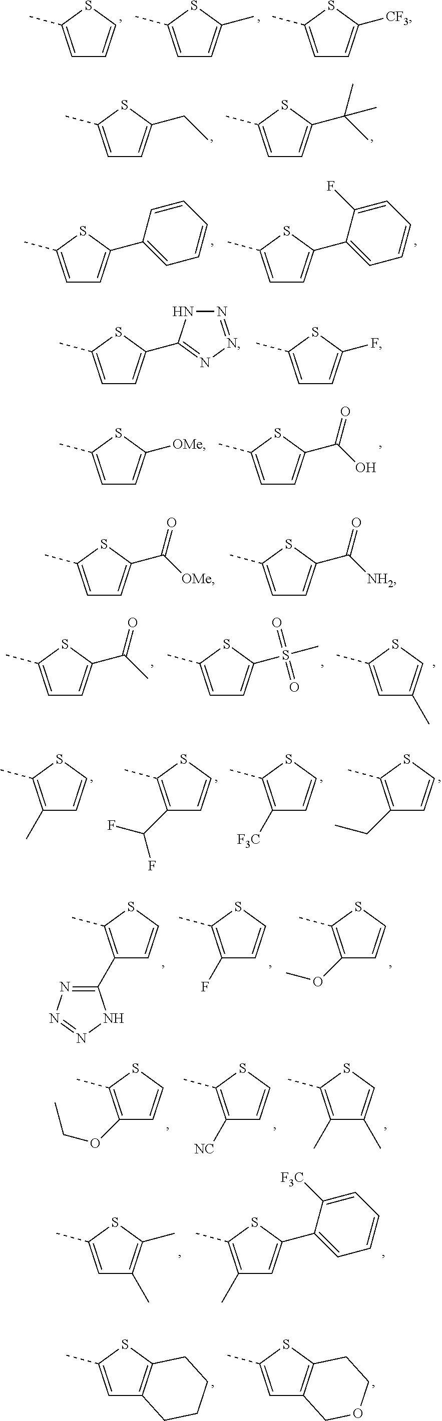

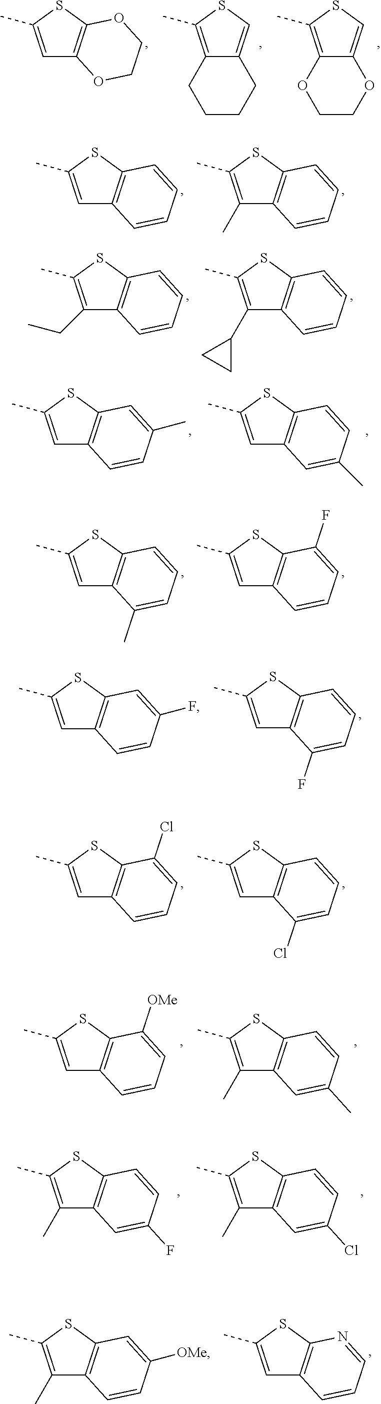

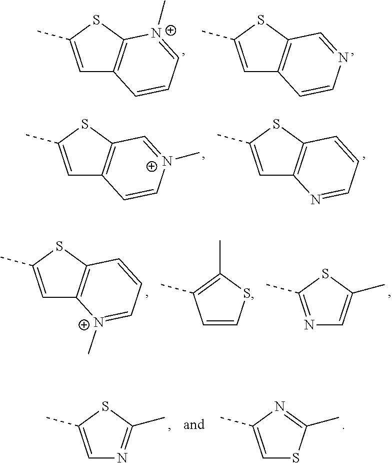

Embodiment 26: The compound of any one of Embodiments 11-25, which is selected from the group consisting of:
5-(2-ethyl-6-methylphenyl)thiophene-2-carboxylic acid;
5-(2,6-diethylphenyl)thiophene-2-carboxylic acid;
5-(2,6-diisopropylphenyl)thiophene-2-carboxylic acid;
5-(2-vinylphenyl)thiophene-2-carboxylic acid;
5-(2-(2-cyano-1-hydroxyethyl)phenyl)thiophene-2-carboxylic acid;
5-(2-(prop-2-yn-1-yl)phenyl)thiophene-2-carboxylic acid;
5-(2-(trifluoromethoxy)phenyl)thiophene-2-carboxylic acid;
5-(2-(methoxymethyl)phenyl)thiophene-2-carboxylic acid;
5-(2-(2-cyanoacetyl)phenyl)thiophene-2-carboxylic acid;
5-(2-(cyanomethoxy)phenyl)thiophene-2-carboxylic acid;
1-(5-(2,6-diethylphenyl)thiophen-2-yl)butane-1,2,3-trione;
(E)-5-(2-(2-cyanovinyl)phenyl)thiophene-2-carboxylic acid;
(Z)-5-(2-(2-cyanovinyl)phenyl)thiophene-2-carboxylic acid;
5-(2-formylphenyl)thiophene-2-carboxylic acid;
5-(2,6-diethylphenyl)-N-hydroxythiophene-2-carboxamide;
5-(2,6-diethylphenyl)-N-methoxythiophene-2-carboxamide;
5-(2-methyl-5-(phenylethynyl)phenyl)thiophene-2-carboxylic acid;
(E)-5-(2-methyl-5-styrylphenyl)thiophene-2-carboxylic acid;
5-(2-methyl-5-phenethylphenyl)thiophene-2-carboxylic acid;
5-(5-benzyl-2-methylphenyl)thiophene-2-carboxylic acid;
5-(4-methyl-[1,1′-biphenyl]-3-yl)thiophene-2-carboxylic acid;
5-(5-cyclopropyl-2-methylphenyl)thiophene-2-carboxylic acid;
5-(2-benzyl-6-methylphenyl)thiophene-2-carboxylic acid;
5-(2-methyl-6-(phenylethynyl)phenyl)thiophene-2-carboxylic acid;
(E)-5-(2-methyl-6-styrylphenyl)thiophene-2-carboxylic acid;
5-(2-methyl-6-phenethylphenyl)thiophene-2-carboxylic acid;
5-(2-cyclopropyl-5-methylphenyl)thiophene-2-carboxylic acid;
5-(2-(N,5-dimethylthiophene-2-carboxamido)-6-methylphenyl)thiophene-2-carboxylic acid;
5-(2-(benzo[b]thiophene-2-carboxamido)-6-methylphenyl)thiophene-2-carboxylic acid;
5-(2-methyl-6-(6-methylbenzo[b]thiophene-2-carboxamido)phenyl)thiophene-2-carboxylic acid;
5-(2-methyl-6-(N-methylbenzo[b]thiophene-2-carboxamido)phenyl)thiophene-2-carboxylic acid;
5-(2-(N,6-dimethylbenzo[b]thiophene-2-carboxamido)-6-methylphenyl)thiophene-2-carboxylic acid;
5-(2-methyl-6-(2-(thiophen-2-yl)acetamido)phenyl)thiophene-2-carboxylic acid;
5-(2-methyl-6-(5-methylfuran-2-carboxamido)phenyl)thiophene-2-carboxylic acid;
5-(2-methyl-6-(3-methylthiophene-2-carboxamido)phenyl)thiophene-2-carboxylic acid;
5-(2-(N,3-dimethylthiophene-2-carboxamido)-6-methylphenyl)thiophene-2-carboxylic acid;
5-(2-methyl-6-(5-methylthiazole-2-carboxamido)phenyl)thiophene-2-carboxylic acid;
5-(2-(N,5-dimethylthiazole-2-carboxamido)-6-methylphenyl)thiophene-2-carboxylic acid;
5-(2-methyl-6-(4-methylthiophene-2-carboxamido)phenyl)thiophene-2-carboxylic acid;
5-(2-(N-butyl-5-methylthiophene-2-carboxamido)-6-methylphenyl)thiophene-2-carboxylic acid;
5-(2-methyl-6-(2-methylthiazole-4-carboxamido)phenyl)thiophene-2-carboxylic acid;
5-(2-methyl-6-(2-methylthiazole-5-carboxamido)phenyl)thiophene-2-carboxylic acid;
5-(2-methyl-6-(2-methylthiophene-3-carboxamido)phenyl)thiophene-2-carboxylic acid;
5-(2-(N,2-dimethylthiazole-4-carboxamido)-6-methylphenyl)thiophene-2-carboxylic acid;
5-(2-methyl-6-(thiophene-2-carboxamido)phenyl)thiophene-2-carboxylic acid5-(2-methyl-6-(thiophene-2-carboxamido)phenyl)thiophene-2-carboxylic acid;
5-(2-methyl-6-(N-methylthiophene-2-carboxamido)phenyl)thiophene-2-carboxylic acid;
5-(2-(N-ethyl-5-methylthiophene-2-carboxamido)-6-methylphenyl)thiophene-2-carboxylic acid;
5-(2-methyl-6-(3-(5-methylthiophen-2-yl)ureido)phenyl)thiophene-2-carboxylic acid5-(2-methyl-6-(3-(5-methylthiophen-2-yl)ureido)phenyl)thiophene-2-carboxylic acid;
5-(2-methyl-6-(5-methyl-N-propylthiophene-2-carboxamido)phenyl)thiophene-2-carboxylic acid;
5-(2-(N-cyclobutyl-5-methylthiophene-2-carboxamido)-6-methylphenyl)thiophene-2-carboxylic acid;
5-(2-(N,5-dimethylthiophene-2-carboxamido)-6-vinylphenyl)thiophene-2-carboxylic acid;
5-(2-(N,5-dimethylthiophene-2-carboxamido)-6-ethylphenyl)thiophene-2-carboxylic acid;
5-(2-methyl-6-(3-(5-methylthiophen-2-yl)-2-oxoimidazolidin-1-yl)phenyl)thiophene-2-carboxylic acid;
5-(2-(5-ethylthiophene-2-carboxamido)-6-methylphenyl)thiophene-2-carboxylic acid5-(2-(5-ethylthiophene-2-carboxamido)-6-methylphenyl)thiophene-2-carboxylic acid;
5-(2-(5-ethyl-N-methylthiophene-2-carboxamido)-6-methylphenyl)thiophene-2-carboxylic acid;
5-(2-(5-methoxythiophene-2-carboxamido)-6-methylphenyl)thiophene-2-carboxylic acid5-(2-(5-methoxythiophene-2-carboxamido)-6-methylphenyl)thiophene-2-carboxylic acid;
5-(2-(5-methoxy-N-methylthiophene-2-carboxamido)-6-methylphenyl)thiophene-2-carboxylic acid;
5-((2-(5-carboxythiophen-2-yl)-3-methylphenyl)carbamoyl)thiophene-2-carboxylic acid;
5-(2-(5-carboxy-N-methylthiophene-2-carboxamido)-6-methylphenyl)thiophene-2-carboxylic acid;
5-(2-(5-(tert-butyl)thiophene-2-carboxamido)-6-methylphenyl)thiophene-2-carboxylic acid;
5-(2-(5-fluorothiophene-2-carboxamido)-6-methylphenyl)thiophene-2-carboxylic acid5-(2-(5-fluorothiophene-2-carboxamido)-6-methylphenyl)thiophene-2-carboxylic acid;
5-(2-methyl-6-(4-methylbenzo[b]thiophene-2-carboxamido)phenyl)thiophene-2-carboxylic acid;
5-(2-(4,5-dimethylthiophene-2-carboxamido)-6-methylphenyl)thiophene-2-carboxylic acid;
5-(2-(6,7-dihydro-4H-thieno[3,2-c]pyran-2-carboxamido)-6-methylphenyl)thiophene-2-carboxylic acid5-(2-(6,7-dihydro-4H-thieno[3,2-c]pyran-2-carboxamido)-6-methylphenyl)thiophene-2-carboxylic acid;
5-(2-methyl-6-(3-methylbenzo[b]thiophene-2-carboxamido)phenyl)thiophene-2-carboxylic acid;
5-(2-methyl-6-(5-(trifluoromethyl)thiophene-2-carboxamido)phenyl)thiophene-2-carboxylic acid;
5-(2-methyl-6-(4,5,6,7-tetrahydrobenzo[b]thiophene-2-carboxamido)phenyl)thiophene-2-carboxylic acid;
5-(2-methyl-6-(N,4,5-trimethylthiophene-2-carboxamido)phenyl)thiophene-2-carboxylic acid5-(2-methyl-6-(N,4,5-trimethylthiophene-2-carboxamido)phenyl)thiophene-2-carboxylic acid;
5-(2-(5-fluoro-N-methylthiophene-2-carboxamido)-6-methylphenyl)thiophene-2-carboxylic acid;
5-(2-(N,4-dimethylbenzo[b]thiophene-2-carboxamido)-6-methylphenyl)thiophene-2-carboxylic acid;
5-(2-methyl-6-(N,3,4-trimethylthiophene-2-carboxamido)phenyl)thiophene-2-carboxylic acid;
5-(2-methyl-6-(N-methyl-4,5,6,7-tetrahydrobenzo[b]thiophene-2-carboxamido)phenyl)thiophene-2-carboxylic acid;
5-(2-methyl-6-(3-methyl-N-propylthiophene-2-carboxamido)phenyl)thiophene-2-carboxylic acid;
5-(2-(N,3-dimethylbenzo[b]thiophene-2-carboxamido)-6-methylphenyl)thiophene-2-carboxylic acid;
5-(2-methyl-6-(N-methyl-2,3-dihydrothieno[2,3-b][1,4]dioxine-6-carboxamido)phenyl)thiophene-2-carboxylic acid;
5-(2-(N,5-dimethylbenzo[b]thiophene-2-carboxamido)-6-methylphenyl)thiophene-2-carboxylic acid;
5-(2-(3,4-dimethylthiophene-2-carboxamido)-6-methylphenyl)thiophene-2-carboxylic acid;
5-(2-methyl-6-(5-methylbenzo[b]thiophene-2-carboxamido)phenyl)thiophene-2-carboxylic acid;
5-(2-(5-acetylthiophene-2-carboxamido)-6-methylphenyl)thiophene-2-carboxylic acid;
5-(2-methyl-6-(4,5,6,7-tetrahydrobenzo[c]thiophene-1-carboxamido)phenyl)thiophene-2-carboxylic acid;
5-(2-methyl-6-(N-methyl-4,5,6,7-tetrahydrobenzo[c]thiophene-1-carboxamido)phenyl)thiophene-2-carboxylic acid;
5-(2-(N-(cyanomethyl)-5-methylthiophene-2-carboxamido)-6-methylphenyl)thiophene-2-carboxylic acid;
5-(2-methyl-6-(5-methyl-N-(prop-2-yn-1-yl)thiophene-2-carboxamido)phenyl)thiophene-2-carboxylic acid;
5-(2-(N-isopropyl-5-methylthiophene-2-carboxamido)-6-methylphenyl)thiophene-2-carboxylic acid
5-(2-(N-cyclopropyl-5-methylthiophene-2-carboxamido)-6-methylphenyl)thiophene-2-carboxylic acid;
5-(2-(5-methylthiophene-2-carboxamido)-6-(trifluoromethyl)phenyl)thiophene-2-carboxylic acid;
5-(2-(N,5-dimethylthiophene-2-carboxamido)-6-(trifluoromethyl)phenyl)thiophene-2-carboxylic acid;
N-(2-(5-(1H-tetrazol-5-yl)thiophen-2-yl)-3-methylphenyl)-N,5-dimethylthiophene-2-carboxamide;
N-(2-(5-(1H-tetrazol-5-yl)thiophen-2-yl)-3-methylphenyl)-N,3-dimethylthiophene-2-carboxamide;
5-((3-methyl-2-(thiophen-2-yl)phenyl)carbamoyl)thiophene-2-carboxylic acid;
5-(methyl(3-methyl-2-(thiophen-2-yl)phenyl)carbamoyl)thiophene-2-carboxylic acid;
5-((2-(5-carbamoylthiophen-2-yl)-3-methylphenyl)(methyl)carbamoyl)thiophene-2-carboxylic acid;
methyl 5-((2-(5-(1H-tetrazol-5-yl)thiophen-2-yl)-3-methylphenyl)(methyl)carbamoyl)thiophene-2-carboxylate;
5-((2-(5-(1H-tetrazol-5-yl)thiophen-2-yl)-3-methylphenyl)(methyl)carbamoyl)thiophene-2-carboxylic acid;
methyl 5-((2-(5-(methoxycarbonyl)thiophen-2-yl)-3-methylphenyl)carbamoyl)thiophene-2-carboxylate;
N-(2-(5-(1H-tetrazol-5-yl)thiophen-2-yl)-3-methylphenyl)-N,3-dimethylbenzo[b]thiophene-2-carboxamide;
5-(2-(5-carbamoyl-N-methylthiophene-2-carboxamido)-6-methylphenyl)thiophene-2-carboxylic acid;
5-(2-methyl-6-(N-methyl-5-(1H-tetrazol-5-yl)thiophene-2-carboxamido)phenyl)thiophene-2-carboxylic acid;
5-(2-(5-acetyl-N-methylthiophene-2-carboxamido)-6-methylphenyl)thiophene-2-carboxylic acid;
5-(2-methyl-6-(N-methyl-5-(trifluoromethyl)thiophene-2-carboxamido)phenyl)thiophene-2-carboxylic acid;
5-(2-(3-ethylbenzo[b]thiophene-2-carboxamido)-6-methylphenyl)thiophene-2-carboxylic acid;
5-(2-methyl-6-(3-(trifluoromethyl)thiophene-2-carboxamido)phenyl)thiophene-2-carboxylic acid;
5-(2-(5-carboxy-N-methylthiophene-2-carboxamido)-4,6-dimethylphenyl)thiophene-2-carboxylic acid;
5-(2-(3-fluorothiophene-2-carboxamido)-6-methylphenyl)thiophene-2-carboxylic acid;
5-(2-(3-cyclopropylbenzo[b]thiophene-2-carboxamido)-6-methylphenyl)thiophene-2-carboxylic acid;
5-(2-(3-methoxy-N-methylthiophene-2-carboxamido)-6-methylphenyl)thiophene-2-carboxylic acid;
5-(2-methyl-6-(N-methyl-2,3-dihydrothieno[3,4-b][1,4]dioxine-5-carboxamido)phenyl)thiophene-2-carboxylic acid;
5-(2-(3-ethyl-N-methylthiophene-2-carboxamido)-6-methylphenyl)thiophene-2-carboxylic acid;
5-(2-(3-ethoxy-N-methylthiophene-2-carboxamido)-6-methylphenyl)thiophene-2-carboxylic acid;
5-(2-(3-ethoxythiophene-2-carboxamido)-6-methylphenyl)thiophene-2-carboxylic acid;
methyl 5-(2-methyl-6-(N-methyl-3-(1H-tetrazol-5-yl)thiophene-2-carboxamido)phenyl)thiophene-2-carboxylate;
5-(2-methyl-6-(N-methyl-3-(1H-tetrazol-5-yl)thiophene-2-carboxamido)phenyl)thiophene-2-carboxylic acid;
5-(2-(5-fluoro-3-methylbenzo[b]thiophene-2-carboxamido)-6-methylphenyl)thiophene-2-carboxylic acid;
5-(2-(5-fluoro-N,3-dimethylbenzo[b]thiophene-2-carboxamido)-6-methylphenyl)thiophene-2-carboxylic acid;
5-(2-(3-ethyl-N-methylbenzo[b]thiophene-2-carboxamido)-6-methylphenyl)thiophene-2-carboxylic acid;
5-(2-(5-chloro-N,3-dimethylbenzo[b]thiophene-2-carboxamido)-6-methylphenyl)thiophene-2-carboxylic acid;
5-(2-(3-cyclopropyl-N-methylbenzo[b]thiophene-2-carboxamido)-6-methylphenyl)thiophene-2-carboxylic acid;
5-(2-(3-ethylthiophene-2-carboxamido)-6-methylphenyl)thiophene-2-carboxylic acid;
5-(2-(3-(difluoromethyl)thiophene-2-carboxamido)-6-methylphenyl)thiophene-2-carboxylic acid;
5-(2-(3-(difluoromethyl)-N-methylthiophene-2-carboxamido)-6-methylphenyl)thiophene-2-carboxylic acid;
5-(2-methyl-6-(thieno[2,3-b]pyridine-2-carboxamido)phenyl)thiophene-2-carboxylic acid;
5-(2-methyl-6-(N-methylthieno[2,3-b]pyridine-2-carboxamido)phenyl)thiophene-2-carboxylic acid;
2-((2-(5-carboxythiophen-2-yl)-3-methylphenyl)(methyl)carbamoyl)-7-methylthieno[2,3-b]pyridin-7-ium;
5-(2-methyl-6-(thieno[3,2-b]pyridine-2-carboxamido)phenyl)thiophene-2-carboxylic acid;
5-(2-methyl-6-(N-methylthieno[3,2-b]pyridine-2-carboxamido)phenyl)thiophene-2-carboxylic acid;
2-((2-(5-carboxythiophen-2-yl)-3-methylphenyl)(methyl)carbamoyl)-4-methylthieno[3,2-b]pyridin-4-ium;
5-(2-(7-fluorobenzo[b]thiophene-2-carboxamido)-6-methylphenyl)thiophene-2-carboxylic acid;
5-(2-(7-fluoro-N-methylbenzo[b]thiophene-2-carboxamido)-6-methylphenyl)thiophene-2-carboxylic acid;
5-(2-(3,5-dimethylbenzo[b]thiophene-2-carboxamido)-6-methylphenyl)thiophene-2-carboxylic acid;
5-(2-methyl-6-(N,3,5-trimethylbenzo[b]thiophene-2-carboxamido)phenyl)thiophene-2-carboxylic acid;
5-(2-methyl-6-(5-phenylthiophene-2-carboxamido)phenyl)thiophene-2-carboxylic acid;
5-(2-methyl-6-(N-methyl-5-phenylthiophene-2-carboxamido)phenyl)thiophene-2-carboxylic acid;
2-((2-(5-carboxythiophen-2-yl)-3-methylphenyl)(methyl)carbamoyl)-6-methylthieno[2,3-c]pyridin-6-ium;
5-(2-(N,3-dimethylthiophene-2-carboxamido)-5-methylphenyl)thiophene-2-carboxylic acid;
5-(2-(N,3-dimethylthiophene-2-carboxamido)-5-ethylphenyl)thiophene-2-carboxylic acid;
5-(2-methyl-6-(N-methyl-5-(methylsulfonyl)thiophene-2-carboxamido)phenyl)thiophene-2-carboxylic acid;
5-(2-(5-(2-fluorophenyl)-N-methylthiophene-2-carboxamido)-6-methylphenyl)thiophene-2-carboxylic acid;
5-(2-(4-fluoro-N-methylbenzo[b]thiophene-2-carboxamido)-6-methylphenyl)thiophene-2-carboxylic acid;
5-(2-(7-chloro-N-methylbenzo[b]thiophene-2-carboxamido)-6-methylphenyl)thiophene-2-carboxylic acid;
5-(2-(4-chloro-N-methylbenzo[b]thiophene-2-carboxamido)-6-methylphenyl)thiophene-2-carboxylic acid;
5-(2-(6-fluoro-N-methylbenzo[b]thiophene-2-carboxamido)-6-methylphenyl)thiophene-2-carboxylic acid;
5-(2-(5-methoxy-N,3-dimethylbenzo[b]thiophene-2-carboxamido)-6-methylphenyl)thiophene-2-carboxylic acid;
5-(2-methyl-6-(N-methylthieno[2,3-c]pyridine-2-carboxamido)phenyl)thiophene-2-carboxylic acid;
5-(6-(N,3-dimethylbenzo[b]thiophene-2-carboxamido)-2,3-dimethylphenyl)thiophene-2-carboxylic acid;
5-(2-(7-methoxy-N-methylbenzo[b]thiophene-2-carboxamido)-6-methylphenyl)thiophene-2-carboxylic acid; and
5-(2-(N,3-dimethyl-5-(2-(trifluoromethyl)phenyl)thiophene-2-carboxamido)-6-methylphenyl)thiophene-2-carboxylic acid.
Embodiment 27: A compound selected from the group consisting of:
5-(4-fluoro-2,6-dimethylphenyl)thiophene-2-carboxylic acid;
5-(2-bromophenyl)thiophene-2-carboxylic acid;
5-(2-cyclohexylphenyl)thiophene-2-carboxylic acid;
5-(2-hydroxyphenyl)thiophene-2-carboxylic acid;
5-(2,3-dimethylphenyl)thiophene-2-carboxylic acid;
5-(2-methoxy-6-methylphenyl)-3-methylthiophene-2-carboxylic acid;
5-(4-amino-2,6-dimethylphenyl)thiophene-2-carboxylic acid;
5-(2-ethyl-6-(5-methylthiophene-2-carbonyl)phenyl)thiophene-2-carboxylic acid;
5-(2-ethyl-6-((5-methylthiophen-2-yl)methyl)phenyl)thiophene-2-carboxylic acid;
5,5-dimethyl-4-oxo-4,5-dihydronaphtho[1,2-b]thiophene-2-carboxylic acid;
5-(2-ethylphenyl)-4-methylthiophene-2-carboxylic acid;
5-(2-ethylphenyl)-1-methyl-1H-pyrazole-3-carboxylic acid;
5-(2-ethylphenyl)-1H-pyrazole-3-carboxylic acid;
methyl 3-(5,5-dimethyl-4,5-dihydronaphtho[1,2-b]thiophen-2-yl)-2,2-dihydroxy-3-oxopropanoate;
methyl 5-(5-bromo-2-methylphenyl)thiophene-2-carboxylate;
1-(4-methyl-3-nitropyridin-2-yl)-4-((4-methylthiazol-2-yl)methyl)piperidin-4-ol;
5-(2-bromo-6-methylphenyl)thiophene-2-carboxylic acid; and
methyl 5-(2-bromo-6-methylphenyl)thiophene-2-carboxylate.

Embodiment 28: A pharmaceutical composition comprising the compound of any one of Embodiments 1-27 and a pharmaceutically acceptable carrier.

Embodiment 29: A method of treating, preventing, and/or ameliorating toxicity caused by a ribosome inactivating protein (RIP) in a subject, the method comprising administering to the subject a therapeutically effective amount of a compound selected from the group consisting of:
(a) a compound of Formula (III) or a pharmaceutically acceptable salt thereof:

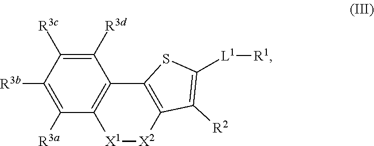

wherein:
   R¹ is a carboxylic acid (—COOH), —C(═O)C(OH)₂C(═O)OR^{A}, a carboxylic acid bioisostere, or a carboxylic acid precursor;
   R² is H;
   R^{3a}, R^{3b}, R^{3c}, and R^{3d} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   L¹ is selected from the group consisting of a bond and optionally substituted C₁-C₂ alkylenyl;
   X¹ is selected from the group consisting of —C(R^{4a})(R^{4b})—, O, and S;
   X² is selected from the group consisting of —C(R^{5a})(R^{5b})— and a bond;
   R^{4a} and R^{4b} are each independently selected from the group consisting of H and optionally substituted C₁-C₆ alkyl,
      wherein no more than one of R^{4a} and R^{4b} is H;
   R^{5a} and R^{5b} are each independently selected from the group consisting of H and optionally substituted C₁-C₆ alkyl,
      or R^{5a} and R^{5b} can combine with the carbon atom to which they are bound to form a carbonyl moiety (C═O);
   R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
   (b) a compound of Formula (II) or a pharmaceutically acceptance salt thereof:

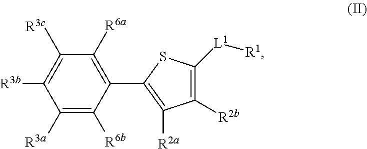

wherein:
   L¹ is selected from the group consisting of a bond and optionally substituted C₁-C₂ alkylenyl;
   R¹ is a carboxylic acid (—COOH), —C(═O)OR^{A}, —[C(═O)]₃R^{A}, —C(═O)C(OH)₂C(═O)OR^{A}, —C(═O)NHOR^{A}, H, a carboxylic acid bioisostere, or a carboxylic acid precursor;
   R^{2a} and R^{2b} are each independently H or optionally substituted C₁-C₆ alkyl;
   R^{3a}, R^{3b}, and R^{3c} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
   R^{6a} and R^{6b} are each independently selected from the group consisting of H, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₆-C₁₀ aryl, optionally substituted C₂-C₈ heteroaryl, C(═O)R^{A}, and

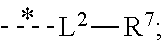

L² is selected from the group consisting of

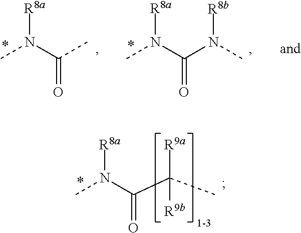

R⁷ is selected from the group consisting of

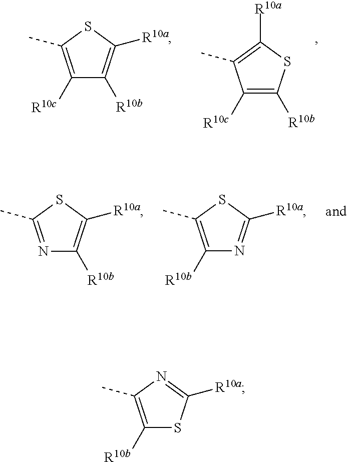

R^{8a} and R^{8b} are each independently selected from the group consisting of H, optionally substituted C₁-C₆ alkyl, and optionally substituted C₃-C₈ cycloalkyl, or R^{8a} and R^{8b} can combine with the atoms to which they are bound to form an optionally substituted C₂-C₈ heterocycloalkyl;
R^{9a} and R^{9b} are each independently selected from the group consisting of H and optionally substituted C₁-C₆ alkyl;
R^{10a}, R^{10b}, and R^{10c} are each independently selected from the group consisting of optionally substituted C₁-C₆ alkyl, optionally substituted C₃-C₈ cycloalkyl, optionally substituted C₆-C₁₀ aryl, optionally substituted C₆-C₈ heteroaryl, halogen, C(═O)R^{A}, C(═O)OR^{A}, C(═O)N(R^{A})(R^{B}), S(═O)R^{A}, S(═O)₂R^{A}, S(═O)N(R^{A})(R^{B}), and S(═O)₂N(R^{A})(R^{B}), or two vicinal substituents selected from the group consisting of R^{10a}, R^{10b}, and R^{10c} can combine with the atoms to which they are bound to form an optionally substituted C₃-C₈ cycloalkyl, optionally substituted C₂-C₈ heterocycloalkyl, optionally substituted C₆-C₁₀ aryl, or optionally substituted C₂-C₈ heteroaryl;
each occurrence of R^{A} and R^{B} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
wherein one of the following applies:
(a) R^{6a} and R^{6b} are each optionally substituted C₁-C₆ alkyl, wherein no more than one of R^{6a} and R^{6b} is optionally substituted C₁ alkyl;
(b) one of R^{6a} and R^{6b} is methyl, and one of R^{6a} and R^{6b} is selected from the group consisting of optionally substituted C₂-C₆ alkenyl, optionally substituted C₂-C₆ alkynyl, optionally substituted C₃-C₈ cycloalkyl, optionally substituted phenyl, and C₁-C₆ alkyl, wherein the optionally substituted alkyl is substituted with at least one optionally substituted phenyl;
(c) one of R^{6a} and R^{6b} is methyl, one of R^{6a} and R^{6b} is H, and one of R^{3a} and R^{3c} is selected from the group consisting of optionally substituted C₁-C₆ alkyl, optionally substituted C₂-C₆ alkenyl, optionally substituted C₂-C₆ alkynyl, optionally substituted C₃-C₈ cycloalkyl, optionally substituted phenyl, and C₁-C₆ alkyl;
(d) one of R^{6a} and R^{6b} is H, and one of R^{6a} and R^{6b} is selected from the group consisting of C₁-C₆ alkyl, optionally substituted C₁-C₆ haloalkoxy, optionally substituted C₂-C₆ alkenyl, optionally substituted C₂-C₆ alkynyl, and C(═O)H, wherein the C₁-C₆ alkyl is substituted with at least one substituent selected from the group consisting of C₂-C₈ alkynyl, C₁-C₆ alkoxy, C═O, and CN; and
(e) one of R^{6a} and R^{6b} is

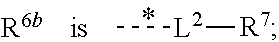

and
(c) a compound selected from the group consisting of:
5-(4-fluoro-2,6-dimethylphenyl)thiophene-2-carboxylic acid;
5-(2-bromophenyl)thiophene-2-carboxylic acid;
5-(2-cyclohexylphenyl)thiophene-2-carboxylic acid;
5-(2-hydroxyphenyl)thiophene-2-carboxylic acid;
5-(2,3-dimethylphenyl)thiophene-2-carboxylic acid;
5-(2-methoxy-6-methylphenyl)-3-methylthiophene-2-carboxylic acid;
5-(4-amino-2,6-dimethylphenyl)thiophene-2-carboxylic acid;
5-(2-ethyl-6-(5-methylthiophene-2-carbonyl)phenyl)thiophene-2-carboxylic acid;
5-(2-ethyl-6-((5-methylthiophen-2-yl)methyl)phenyl)thiophene-2-carboxylic acid;
5,5-dimethyl-4-oxo-4,5-dihydronaphtho[1,2-b]thiophene-2-carboxylic acid;
5-(2-ethylphenyl)-4-methylthiophene-2-carboxylic acid;
5-(2-ethylphenyl)-1-methyl-1H-pyrazole-3-carboxylic acid;
5-(2-ethylphenyl)-1H-pyrazole-3-carboxylic acid;
methyl 3-(5,5-dimethyl-4,5-dihydronaphtho[1,2-b]thiophen-2-yl)-2,2-dihydroxy-3-oxopropanoate;
methyl 5-(5-bromo-2-methylphenyl)thiophene-2-carboxylate;
1-(4-methyl-3-nitropyridin-2-yl)-4-((4-methylthiazol-2-yl)methyl)piperidin-4-ol;
5-(2-bromo-6-methylphenyl)thiophene-2-carboxylic acid; and
methyl 5-(2-bromo-6-methylphenyl)thiophene-2-carboxylate.

Embodiment 30: The method of Embodiment 29, wherein the RIP is ricin or Shiga toxin 2a (Stx2a).

Embodiment 31: The method of Embodiment 29 or 30, wherein the compound inhibitions depurination activity of the RIP.

Embodiment 32: The method of any one of Embodiments 29-31, wherein the compound inhibits interaction of the RIP with a ribosome.

Embodiment 33: The method of any one of Embodiments 29-32, wherein the compound inhibits the interaction of an active A chain (RTA) of the RIP with a ribosome.

Embodiment 34: The method of Embodiment 32 or 33, wherein the compound binds to the ribosome binding site of the RTA.

Embodiment 35: The method of any one of Embodiments 29-34, wherein the ribosome inactivating protein (RIP) is either a type I or type II RIP.

Embodiment 36: The method of any one of Embodiments 29-25, wherein the compound is administered as a pharmaceutical composition to the subject.

Embodiment 37: The method of any one of Embodiments 29-26, wherein the subject is administered at least one additional agent useful for treating, ameliorating, and/or preventing the toxicity caused by RIP.

Embodiment 38: The method of Embodiment 37, wherein the at least one additional agent is selected from the group consisting of immunotherapeutics and vaccines.

Embodiment 39: The method of Embodiment 37 or 38, wherein administering the compound to the subject allows for administering a lower dose of the at least one additional agent as compared to the dose of the at least one additional agent alone that is required to achieve similar results in treating, ameliorating, and/or preventing toxicity caused by RIP.

Embodiment 40: The method of any one of Embodiments 37-39, wherein the compound and the at least one additional agent are co-administered to the subject.

Embodiment 41: The method of any one of Embodiments 37-40, wherein the compound and the at least one additional agent are co-formulated.

Embodiment 42: The method of any one of Embodiments 29-41, wherein the compound of Formula (III) or Formula (II) is selected from the group consisting of:
8-chloro-4H-thieno[3,2-c]thiochromene-2-carbohydrazide;
methyl 8-fluoro-4H-thieno[3,2-c]chromene-2-carboxylate;
1-(8-chloro-4H-thieno[3,2-c]thiochromen-2-yl)ethan-1-one;
8-chloro-4H-thieno[3,2-c]thiochromene-2-carboxylic acid;
4H-thieno[3,2-c]chromene-2-carboxylic acid;
8-fluoro-4H-thieno[3,2-c]thiochromene-2-carboxylic acid;
benzo[b]thieno[2,3-d]thiophene-2-carboxylic acid;
5,5-dimethyl-4,5-dihydronaphtho[1,2-b]thiophene-2-carboxylic acid;
5-(2-ethyl-6-methylphenyl)thiophene-2-carboxylic acid;
5-(2,6-diethylphenyl)thiophene-2-carboxylic acid;
5,5-dimethyl-4,5-dihydronaphtho[1,2-b]thiophene-2-carboxamide;
5,5-dimethyl-4,5-dihydronaphtho[1,2-b]thiophene-2-carbonitrile;
5-(5,5-dimethyl-4,5-dihydronaphtho[1,2-b]thiophen-2-yl)-2H-tetrazole; and
5,5-dimethyl-N-(methylsulfonyl)-4,5-dihydronaphtho[1,2-b]thiophene-2-carboxamide;
5,5-dimethyl-4-oxo-4,5-dihydronaphtho[1,2-b]thiophene-2-carboxylic acid;
methyl 3-(5,5-dimethyl-4,5-dihydronaphtho[1,2-b]thiophen-2-yl)-2,2-dihydroxy-3-oxopropanoate;
5-(2,6-diisopropylphenyl)thiophene-2-carboxylic acid;
5-(2-vinylphenyl)thiophene-2-carboxylic acid;
5-(2-(2-cyano-1-hydroxyethyl)phenyl)thiophene-2-carboxylic acid;
5-(2-(prop-2-yn-1-yl)phenyl)thiophene-2-carboxylic acid;
5-(2-(trifluoromethoxy)phenyl)thiophene-2-carboxylic acid;
5-(2-(methoxymethyl)phenyl)thiophene-2-carboxylic acid;
5-(2-(2-cyanoacetyl)phenyl)thiophene-2-carboxylic acid;
5-(2-(cyanomethoxy)phenyl)thiophene-2-carboxylic acid;
1-(5-(2,6-diethylphenyl)thiophen-2-yl)butane-1,2,3-trione;
(E)-5-(2-(2-cyanovinyl)phenyl)thiophene-2-carboxylic acid;
(Z)-5-(2-(2-cyanovinyl)phenyl)thiophene-2-carboxylic acid;
5-(2-formylphenyl)thiophene-2-carboxylic acid;
5-(2,6-diethylphenyl)-N-hydroxythiophene-2-carboxamide;
5-(2,6-diethylphenyl)-N-methoxythiophene-2-carboxamide;
5-(2-methyl-5-(phenylethynyl)phenyl)thiophene-2-carboxylic acid;
(E)-5-(2-methyl-5-styrylphenyl)thiophene-2-carboxylic acid;
5-(2-methyl-5-phenethylphenyl)thiophene-2-carboxylic acid;
5-(5-benzyl-2-methylphenyl)thiophene-2-carboxylic acid;
5-(4-methyl-[1,1′-biphenyl]-3-yl)thiophene-2-carboxylic acid;
5-(5-cyclopropyl-2-methylphenyl)thiophene-2-carboxylic acid;
5-(2-benzyl-6-methylphenyl)thiophene-2-carboxylic acid;
5-(2-methyl-6-(phenylethynyl)phenyl)thiophene-2-carboxylic acid;
(E)-5-(2-methyl-6-styrylphenyl)thiophene-2-carboxylic acid;
5-(2-methyl-6-phenethylphenyl)thiophene-2-carboxylic acid;
5-(2-cyclopropyl-5-methylphenyl)thiophene-2-carboxylic acid;
5-(2-(N,5-dimethylthiophene-2-carboxamido)-6-methylphenyl)thiophene-2-carboxylic acid;
5-(2-(benzo[b]thiophene-2-carboxamido)-6-methylphenyl)thiophene-2-carboxylic acid;
5-(2-methyl-6-(6-methylbenzo[b]thiophene-2-carboxamido)phenyl)thiophene-2-carboxylic acid;
5-(2-methyl-6-(N-methylbenzo[b]thiophene-2-carboxamido)phenyl)thiophene-2-carboxylic acid;
5-(2-(N,6-dimethylbenzo[b]thiophene-2-carboxamido)-6-methylphenyl)thiophene-2-carboxylic acid;
5-(2-methyl-6-(2-(thiophen-2-yl)acetamido)phenyl)thiophene-2-carboxylic acid;
5-(2-methyl-6-(5-methylfuran-2-carboxamido)phenyl)thiophene-2-carboxylic acid;
5-(2-methyl-6-(3-methylthiophene-2-carboxamido)phenyl)thiophene-2-carboxylic acid;
5-(2-(N,3-dimethylthiophene-2-carboxamido)-6-methylphenyl)thiophene-2-carboxylic acid;
5-(2-methyl-6-(5-methylthiazole-2-carboxamido)phenyl)thiophene-2-carboxylic acid;
5-(2-(N,5-dimethylthiazole-2-carboxamido)-6-methylphenyl)thiophene-2-carboxylic acid;
5-(2-methyl-6-(4-methylthiophene-2-carboxamido)phenyl)thiophene-2-carboxylic acid;
5-(2-(N-butyl-5-methylthiophene-2-carboxamido)-6-methylphenyl)thiophene-2-carboxylic acid;
5-(2-methyl-6-(2-methylthiazole-4-carboxamido)phenyl)thiophene-2-carboxylic acid;
5-(2-methyl-6-(2-methylthiazole-5-carboxamido)phenyl)thiophene-2-carboxylic acid;
5-(2-methyl-6-(2-methylthiophene-3-carboxamido)phenyl)thiophene-2-carboxylic acid;
5-(2-(N,2-dimethylthiazole-4-carboxamido)-6-methylphenyl)thiophene-2-carboxylic acid;
5-(2-methyl-6-(thiophene-2-carboxamido)phenyl)thiophene-2-carboxylic acid5-(2-methyl-6-(thiophene-2-carboxamido)phenyl)thiophene-2-carboxylic acid;
5-(2-methyl-6-(N-methylthiophene-2-carboxamido)phenyl)thiophene-2-carboxylic acid;
5-(2-(N-ethyl-5-methylthiophene-2-carboxamido)-6-methylphenyl)thiophene-2-carboxylic acid;
5-(2-methyl-6-(3-(5-methylthiophen-2-yl)ureido)phenyl)thiophene-2-carboxylic acid5-(2-methyl-6-(3-(5-methylthiophen-2-yl)ureido)phenyl)thiophene-2-carboxylic acid;
5-(2-methyl-6-(5-methyl-N-propylthiophene-2-carboxamido)phenyl)thiophene-2-carboxylic acid;
5-(2-(N-cyclobutyl-5-methylthiophene-2-carboxamido)-6-methylphenyl)thiophene-2-carboxylic acid;
5-(2-(N,5-dimethylthiophene-2-carboxamido)-6-vinylphenyl)thiophene-2-carboxylic acid;
5-(2-(N,5-dimethylthiophene-2-carboxamido)-6-ethylphenyl)thiophene-2-carboxylic acid;
5-(2-methyl-6-(3-(5-methylthiophen-2-yl)-2-oxoimidazolidin-1-yl)phenyl)thiophene-2-carboxylic acid;
5-(2-(5-ethylthiophene-2-carboxamido)-6-methylphenyl)thiophene-2-carboxylic acid5-(2-(5-ethylthiophene-2-carboxamido)-6-methylphenyl)thiophene-2-carboxylic acid;
5-(2-(5-ethyl-N-methylthiophene-2-carboxamido)-6-methylphenyl)thiophene-2-carboxylic acid;
5-(2-(5-methoxythiophene-2-carboxamido)-6-methylphenyl)thiophene-2-carboxylic acid5-(2-(5-methoxythiophene-2-carboxamido)-6-methylphenyl)thiophene-2-carboxylic acid;
5-(2-(5-methoxy-N-methylthiophene-2-carboxamido)-6-methylphenyl)thiophene-2-carboxylic acid;
5-((2-(5-carboxythiophen-2-yl)-3-methylphenyl)carbamoyl)thiophene-2-carboxylic acid;
5-(2-(5-carboxy-N-methylthiophene-2-carboxamido)-6-methylphenyl)thiophene-2-carboxylic acid;
5-(2-(5-(tert-butyl)thiophene-2-carboxamido)-6-methylphenyl)thiophene-2-carboxylic acid;
5-(2-(5-fluorothiophene-2-carboxamido)-6-methylphenyl)thiophene-2-carboxylic acid5-(2-(5-fluorothiophene-2-carboxamido)-6-methylphenyl)thiophene-2-carboxylic acid;
5-(2-methyl-6-(4-methylbenzo[b]thiophene-2-carboxamido)phenyl)thiophene-2-carboxylic acid;
5-(2-(4,5-dimethylthiophene-2-carboxamido)-6-methylphenyl)thiophene-2-carboxylic acid;
5-(2-(6,7-dihydro-4H-thieno[3,2-c]pyran-2-carboxamido)-6-methylphenyl)thiophene-2-carboxylic acid5-(2-(6,7-dihydro-4H-thieno[3,2-c]pyran-2-carboxamido)-6-methylphenyl)thiophene-2-carboxylic acid;
5-(2-methyl-6-(3-methylbenzo[b]thiophene-2-carboxamido)phenyl)thiophene-2-carboxylic acid;
5-(2-methyl-6-(5-(trifluoromethyl)thiophene-2-carboxamido)phenyl)thiophene-2-carboxylic acid;
5-(2-methyl-6-(4,5,6,7-tetrahydrobenzo[b]thiophene-2-carboxamido)phenyl)thiophene-2-carboxylic acid;
5-(2-methyl-6-(N,4,5-trimethylthiophene-2-carboxamido)phenyl)thiophene-2-carboxylic acid5-(2-methyl-6-(N,4,5-trimethylthiophene-2-carboxamido)phenyl)thiophene-2-carboxylic acid;
5-(2-(5-fluoro-N-methylthiophene-2-carboxamido)-6-methylphenyl)thiophene-2-carboxylic acid;
5-(2-(N,4-dimethylbenzo[b]thiophene-2-carboxamido)-6-methylphenyl)thiophene-2-carboxylic acid;
5-(2-methyl-6-(N,3,4-trimethylthiophene-2-carboxamido)phenyl)thiophene-2-carboxylic acid;
5-(2-methyl-6-(N-methyl-4,5,6,7-tetrahydrobenzo[b]thiophene-2-carboxamido)phenyl)thiophene-2-carboxylic acid;
5-(2-methyl-6-(3-methyl-N-propylthiophene-2-carboxamido)phenyl)thiophene-2-carboxylic acid;
5-(2-(N,3-dimethylbenzo[b]thiophene-2-carboxamido)-6-methylphenyl)thiophene-2-carboxylic acid;
5-(2-methyl-6-(N-methyl-2,3-dihydrothieno[2,3-b][1,4]dioxine-6-carboxamido)phenyl)thiophene-2-carboxylic acid;
5-(2-(N,5-dimethylbenzo[b]thiophene-2-carboxamido)-6-methylphenyl)thiophene-2-carboxylic acid;
5-(2-(3,4-dimethylthiophene-2-carboxamido)-6-methylphenyl)thiophene-2-carboxylic acid;
5-(2-methyl-6-(5-methylbenzo[b]thiophene-2-carboxamido)phenyl)thiophene-2-carboxylic acid;
5-(2-(5-acetylthiophene-2-carboxamido)-6-methylphenyl)thiophene-2-carboxylic acid;
5-(2-methyl-6-(4,5,6,7-tetrahydrobenzo[c]thiophene-1-carboxamido)phenyl)thiophene-2-carboxylic acid;
5-(2-methyl-6-(N-methyl-4,5,6,7-tetrahydrobenzo[c]thiophene-1-carboxamido)phenyl)thiophene-2-carboxylic acid;
5-(2-(N-(cyanomethyl)-5-methylthiophene-2-carboxamido)-6-methylphenyl)thiophene-2-carboxylic acid;
5-(2-methyl-6-(5-methyl-N-(prop-2-yn-1-yl)thiophene-2-carboxamido)phenyl)thiophene-2-carboxylic acid;
5-(2-(N-isopropyl-5-methylthiophene-2-carboxamido)-6-methylphenyl)thiophene-2-carboxylic acid
5-(2-(N-cyclopropyl-5-methylthiophene-2-carboxamido)-6-methylphenyl)thiophene-2-carboxylic acid;
5-(2-(5-methylthiophene-2-carboxamido)-6-(trifluoromethyl)phenyl)thiophene-2-carboxylic acid;
5-(2-(N,5-dimethylthiophene-2-carboxamido)-6-(trifluoromethyl)phenyl)thiophene-2-carboxylic acid;
N-(2-(5-(1H-tetrazol-5-yl)thiophen-2-yl)-3-methylphenyl)-N,5-dimethylthiophene-2-carboxamide;
N-(2-(5-(1H-tetrazol-5-yl)thiophen-2-yl)-3-methylphenyl)-N,3-dimethylthiophene-2-carboxamide;
5-((3-methyl-2-(thiophen-2-yl)phenyl)carbamoyl)thiophene-2-carboxylic acid;
5-(methyl(3-methyl-2-(thiophen-2-yl)phenyl)carbamoyl)thiophene-2-carboxylic acid;
5-((2-(5-carbamoylthiophen-2-yl)-3-methylphenyl)(methyl)carbamoyl)thiophene-2-carboxylic acid;
methyl 5-((2-(5-(1H-tetrazol-5-yl)thiophen-2-yl)-3-methylphenyl)(methyl)carbamoyl)thiophene-2-carboxylate;
5-((2-(5-(1H-tetrazol-5-yl)thiophen-2-yl)-3-methylphenyl)(methyl)carbamoyl)thiophene-2-carboxylic acid;
methyl 5-((2-(5-(methoxycarbonyl)thiophen-2-yl)-3-methylphenyl)carbamoyl)thiophene-2-carboxylate;
N-(2-(5-(1H-tetrazol-5-yl)thiophen-2-yl)-3-methylphenyl)-N,3-dimethylbenzo[b]thiophene-2-carboxamide;
5-(2-(5-carbamoyl-N-methylthiophene-2-carboxamido)-6-methylphenyl)thiophene-2-carboxylic acid;
5-(2-methyl-6-(N-methyl-5-(1H-tetrazol-5-yl)thiophene-2-carboxamido)phenyl)thiophene-2-carboxylic acid;
5-(2-(5-acetyl-N-methylthiophene-2-carboxamido)-6-methylphenyl)thiophene-2-carboxylic acid;
5-(2-methyl-6-(N-methyl-5-(trifluoromethyl)thiophene-2-carboxamido)phenyl)thiophene-2-carboxylic acid;
5-(2-(3-ethylbenzo[b]thiophene-2-carboxamido)-6-methylphenyl)thiophene-2-carboxylic acid;
5-(2-methyl-6-(3-(trifluoromethyl)thiophene-2-carboxamido)phenyl)thiophene-2-carboxylic acid;
5-(2-(5-carboxy-N-methylthiophene-2-carboxamido)-4,6-dimethylphenyl)thiophene-2-carboxylic acid;
5-(2-(3-fluorothiophene-2-carboxamido)-6-methylphenyl)thiophene-2-carboxylic acid;
5-(2-(3-cyclopropylbenzo[b]thiophene-2-carboxamido)-6-methylphenyl)thiophene-2-carboxylic acid;
5-(2-(3-methoxy-N-methylthiophene-2-carboxamido)-6-methylphenyl)thiophene-2-carboxylic acid;
5-(2-methyl-6-(N-methyl-2,3-dihydrothieno[3,4-b][1,4]dioxine-5-carboxamido)phenyl)thiophene-2-carboxylic acid;
5-(2-(3-ethyl-N-methylthiophene-2-carboxamido)-6-methylphenyl)thiophene-2-carboxylic acid;
5-(2-(3-ethoxy-N-methylthiophene-2-carboxamido)-6-methylphenyl)thiophene-2-carboxylic acid;
5-(2-(3-ethoxythiophene-2-carboxamido)-6-methylphenyl)thiophene-2-carboxylic acid;
methyl 5-(2-methyl-6-(N-methyl-3-(1H-tetrazol-5-yl)thiophene-2-carboxamido)phenyl)thiophene-2-carboxylate;
5-(2-methyl-6-(N-methyl-3-(1H-tetrazol-5-yl)thiophene-2-carboxamido)phenyl)thiophene-2-carboxylic acid;
5-(2-(5-fluoro-3-methylbenzo[b]thiophene-2-carboxamido)-6-methylphenyl)thiophene-2-carboxylic acid;
5-(2-(5-fluoro-N,3-dimethylbenzo[b]thiophene-2-carboxamido)-6-methylphenyl)thiophene-2-carboxylic acid;
5-(2-(3-ethyl-N-methylbenzo[b]thiophene-2-carboxamido)-6-methylphenyl)thiophene-2-carboxylic acid;
5-(2-(5-chloro-N,3-dimethylbenzo[b]thiophene-2-carboxamido)-6-methylphenyl)thiophene-2-carboxylic acid;
5-(2-(3-cyclopropyl-N-methylbenzo[b]thiophene-2-carboxamido)-6-methylphenyl)thiophene-2-carboxylic acid;
5-(2-(3-ethylthiophene-2-carboxamido)-6-methylphenyl)thiophene-2-carboxylic acid;
5-(2-(3-(difluoromethyl)thiophene-2-carboxamido)-6-methylphenyl)thiophene-2-carboxylic acid;
5-(2-(3-(difluoromethyl)-N-methylthiophene-2-carboxamido)-6-methylphenyl)thiophene-2-carboxylic acid;
5-(2-methyl-6-(thieno[2,3-b]pyridine-2-carboxamido)phenyl)thiophene-2-carboxylic acid;
5-(2-methyl-6-(N-methylthieno[2,3-b]pyridine-2-carboxamido)phenyl)thiophene-2-carboxylic acid;
2-((2-(5-carboxythiophen-2-yl)-3-methylphenyl)(methyl)carbamoyl)-7-methylthieno[2,3-b]pyridin-7-ium;
5-(2-methyl-6-(thieno[3,2-b]pyridine-2-carboxamido)phenyl)thiophene-2-carboxylic acid;
5-(2-methyl-6-(N-methylthieno[3,2-b]pyridine-2-carboxamido)phenyl)thiophene-2-carboxylic acid;
2-((2-(5-carboxythiophen-2-yl)-3-methylphenyl)(methyl)carbamoyl)-4-methylthieno[3,2-b]pyridin-4-ium;
5-(2-(7-fluorobenzo[b]thiophene-2-carboxamido)-6-methylphenyl)thiophene-2-carboxylic acid;
5-(2-(7-fluoro-N-methylbenzo[b]thiophene-2-carboxamido)-6-methylphenyl)thiophene-2-carboxylic acid;
5-(2-(3,5-dimethylbenzo[b]thiophene-2-carboxamido)-6-methylphenyl)thiophene-2-carboxylic acid;
5-(2-methyl-6-(N,3,5-trimethylbenzo[b]thiophene-2-carboxamido)phenyl)thiophene-2-carboxylic acid;
5-(2-methyl-6-(5-phenylthiophene-2-carboxamido)phenyl)thiophene-2-carboxylic acid;
5-(2-methyl-6-(N-methyl-5-phenylthiophene-2-carboxamido)phenyl)thiophene-2-carboxylic acid;
2-((2-(5-carboxythiophen-2-yl)-3-methylphenyl)(methyl)carbamoyl)-6-methylthieno[2,3-c]pyridin-6-ium;
5-(2-(N,3-dimethylthiophene-2-carboxamido)-5-methylphenyl)thiophene-2-carboxylic acid;
5-(2-(N,3-dimethylthiophene-2-carboxamido)-5-ethylphenyl)thiophene-2-carboxylic acid;
5-(2-methyl-6-(N-methyl-5-(methylsulfonyl)thiophene-2-carboxamido)phenyl)thiophene-2-carboxylic acid;
5-(2-(5-(2-fluorophenyl)-N-methylthiophene-2-carboxamido)-6-methylphenyl)thiophene-2-carboxylic acid;
5-(2-(4-fluoro-N-methylbenzo[b]thiophene-2-carboxamido)-6-methylphenyl)thiophene-2-carboxylic acid;
5-(2-(7-chloro-N-methylbenzo[b]thiophene-2-carboxamido)-6-methylphenyl)thiophene-2-carboxylic acid;
5-(2-(4-chloro-N-methylbenzo[b]thiophene-2-carboxamido)-6-methylphenyl)thiophene-2-carboxylic acid;
5-(2-(6-fluoro-N-methylbenzo[b]thiophene-2-carboxamido)-6-methylphenyl)thiophene-2-carboxylic acid;
5-(2-(5-methoxy-N,3-dimethylbenzo[b]thiophene-2-carboxamido)-6-methylphenyl)thiophene-2-carboxylic acid;
5-(2-methyl-6-(N-methylthieno[2,3-c]pyridine-2-carboxamido)phenyl)thiophene-2-carboxylic acid;
5-(6-(N,3-dimethylbenzo[b]thiophene-2-carboxamido)-2,3-dimethylphenyl)thiophene-2-carboxylic acid;
5-(2-(7-methoxy-N-methylbenzo[b]thiophene-2-carboxamido)-6-methylphenyl)thiophene-2-carboxylic acid; and
5-(2-(N,3-dimethyl-5-(2-(trifluoromethyl)phenyl)thiophene-2-carboxamido)-6-methylphenyl)thiophene-2-carboxylic acid.

Embodiment 43: The method of any one of Embodiments 29-42, wherein the subject is a mammal.

Embodiment 44: The method of Embodiment 43, wherein the subject is a human.

Embodiment 45: A method for measuring binding affinity of a small molecule for an active A chain (RTA) of a ribosome inactivating protein (RIP), the method comprising:
(a) contacting a small molecule with a mixture comprising a fluorescently labeled P11 polypeptide (SEQ ID NO:1) and RTA to provide a displacing mixture;
(b) measuring fluorescence of the displacing mixture to provide a displaced fluorescence mixture;
(c) measuring fluorescence of a control sample comprising the fluorescently labeled P11 polypeptide (SEQ ID NO:1) and RTA, and lacking the small molecule, to provide a control measurement; and
(d) comparing the displaced fluorescence measurement and the control fluorescence measurement.

Embodiment 46: The method of Embodiment 45, wherein more than one occurrence of each of steps (a)-(d) is performed.

Embodiment 47: The method of Embodiment 46, wherein each occurrence of steps (a)-(d) independently occurs with a concentration of the small molecule ranging from about 1 nM to about 1000 μM, optionally wherein the concentration of the small molecule is selected from the group consisting of about 0.001, about 0.01, about 0.1, about 1, about 5, about 10, about 20, about 40, about 80, about 125, about 160, about 250, about 500, or about 1000 μM.

Embodiment 48: The method of Embodiment 46 or 47, wherein each independent occurrence of steps (a)-(d) is performed in a well of a microplate, optionally wherein the microplate comprises a 96-well microplate.

Embodiment 49: The method of any one of Embodiments 45-48, wherein the RTA and fluorescently labeled P11 polypeptide (SEQ ID NO:1) have a molar ratio of about 3:1, optionally wherein the RTA has a concentration of about 3 μM and the fluorescently labeled P11 polypeptide (SEQ ID NO:1) has a concentration of about 1 μM.

Embodiment 50: The method of any one of Embodiments 45-49, wherein the fluorescent label comprises a BODIPY dye, optionally wherein the BODIPY dye comprises and/or is prepared using a BODIPY TMR-X N-hydroxysuccinimide ester.

Embodiment 51: The method of any one of Embodiments 45-50, wherein the contacting step further comprises at least one selected from the group consisting of centrifuging the displacing mixture one or more times and incubating the displacing mixture without significant light exposure.

Embodiment 52: The method of any one of Embodiments 45-51, wherein each measuring step comprises use of an excitation filter of about 530/25 nm and an emission filter of about 590/35 nm.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A compound of Formula (I) or a pharmaceutically acceptable salt thereof: 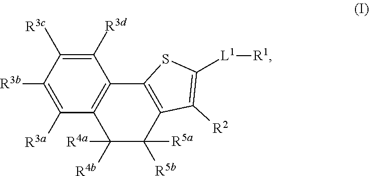
wherein:
R¹ is a carboxylic acid (—COOH), —C(═O)C(OH)₂C(═O)OR^{A}, a carboxylic acid bioisostere, or a carboxylic acid precursor;
R² is H;
R^{3a}, R^{3b}, R^{3c}, and R^{3d} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
L¹ is selected from the group consisting of a bond and optionally substituted C₁-C₂ alkylenyl;
R^{4a} and R^{4b} are each independently selected from the group consisting of H and optionally substituted C₁-C₆ alkyl,
wherein no more than one of R^{4a} and R^{4b} is H;
R^{5a} and R^{5b} are each independently selected from the group consisting of H and optionally substituted C₁-C₆ alkyl,
or R^{5a} and R^{5b} can combine with the carbon atom to which they are bound to form a carbonyl moiety (C═O);
R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
wherein the compound is not 5,5-dimethyl-4,5-dihydronaphtho[1,2-b]thiophene-2-carboxylic acid.

2. The compound of claim 1, wherein R¹ is selected from the group consisting of 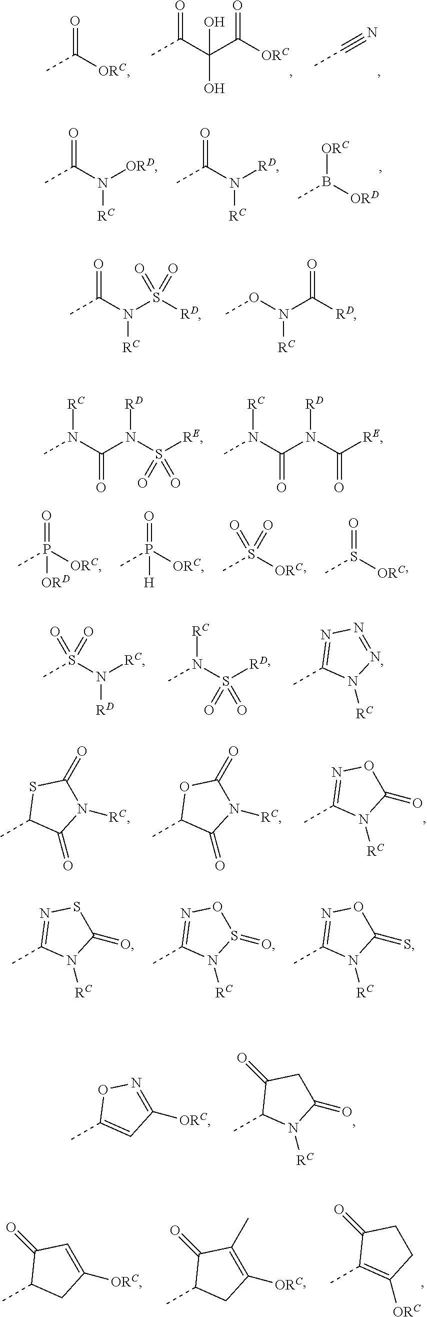 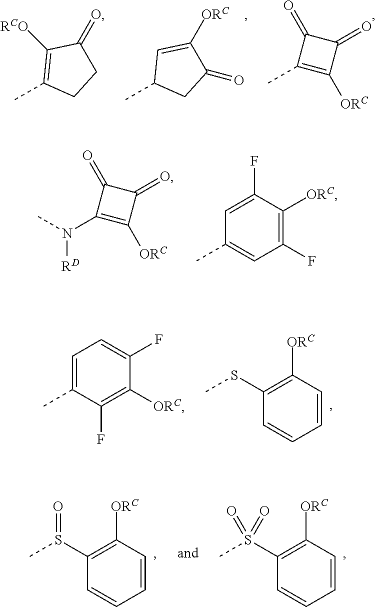
wherein R^{C}, R^{D}, and R^{E}, if present, are each independently selected from the group consisting of H, optionally substituted C₁-C₆ alkyl, and C(═O)(optionally substituted C₁-C₆ alkyl).

3. The compound of claim 1, wherein at least one of the following applies:
(a) R¹ is selected from the group consisting of 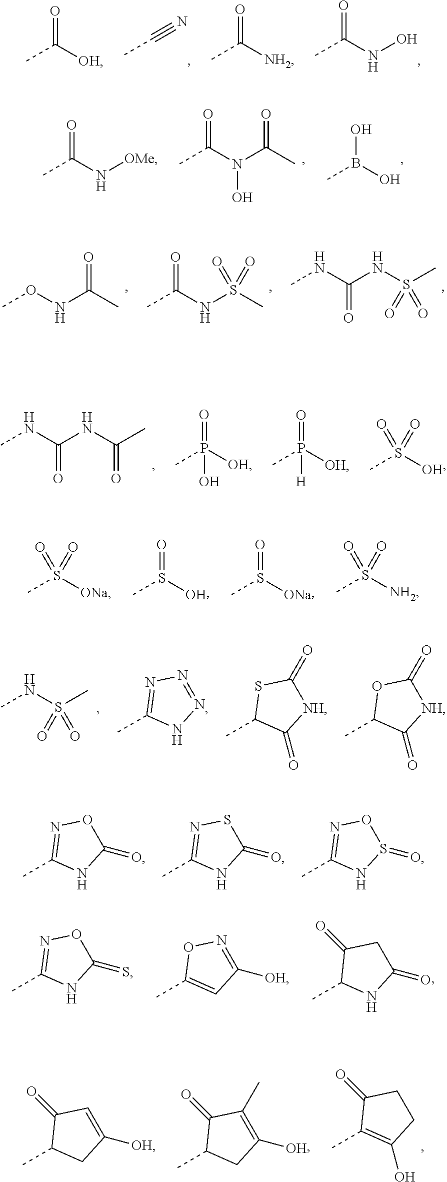 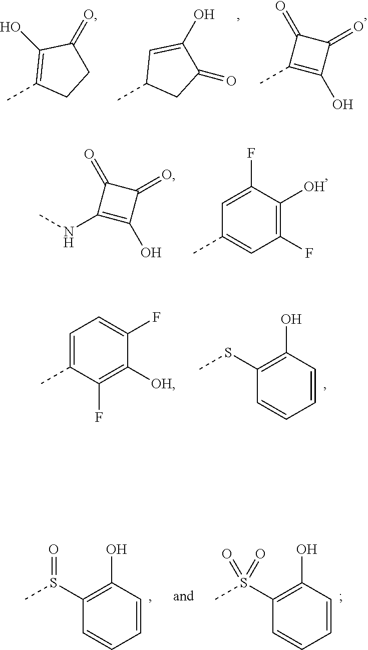
(b) L¹ is a bond;
(c) R^{4a} and R^{4b} are each independently CH₃;
(d) R^{5a} and R^{5b} are each independently H;
(e) R^{3a}, R^{3b}, R^{3c}, and R^{3d} are each independently selected from the group consisting of H, F, Cl, Br, CH₃, OCH₃, and N(CH₃)₂, optionally wherein at least three of R^{3a}R^{3b}, R^{3c}, and R^{3d} are H.

4. The compound of claim 1, wherein one of the following applies:
(a) R^{3b} is selected from the group consisting of F, Cl, Br, CH₃, OCH₃, and N(CH₃) and R^{3a}, R^{3c}, and R^{3d} are each H; and
(b) R^{3c} is selected from the group consisting of F, Cl, Br, CH₃, OCH₃, and N(CH₃) and R^{3a}, R^{3b}, and R^{3d} are each H.

5. The compound of claim 1, which is selected from the group consisting of:
5,5-dimethyl-4,5-dihydronaphtho[1,2-b]thiophene-2-carboxamide;
5,5-dimethyl-4,5-dihydronaphtho[1,2-b]thiophene-2-carbonitrile;
5-(5,5-dimethyl-4,5-dihydronaphtho[1,2-b]thiophen-2-yl)-2H-tetrazole;
5,5-dimethyl-N-(methylsulfonyl)-4,5-dihydronaphtho[1,2-b]thiophene-2-carboxamide
5,5-dimethyl-4-oxo-4,5-dihydronaphtho[1,2-b]thiophene-2-carboxylic acid; and
methyl 3-(5,5-dimethyl-4,5-dihydronaphtho[1,2-b]thiophen-2-yl)-2,2-dihydroxy-3-oxopropanoate.

6. A compound of Formula (II) or a pharmaceutically acceptable salt thereof: 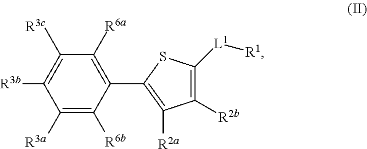
wherein:
L¹ is selected from the group consisting of a bond and optionally substituted C₁-C₂ alkylenyl;
R¹ is a carboxylic acid (—COOH), —C(═O)OR^{A}, —[C(═O)]₃R^{A}, —C(═O)C(OH)₂C(═O)OR^{A}, —C(═O)NHOR^{A}, H, a carboxylic acid bioisostere, or a carboxylic acid precursor;
R^{2a} and R^{2b} are each independently H or optionally substituted C₁-C₆ alkyl;
R^{3a}, R^{3b}, and R^{3c} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
R^{6a} and R^{6b} are each independently selected from the group consisting of H, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₆-C₁₀ aryl, optionally substituted C₂-C₈ heteroaryl, C(═O)R^{A}, and 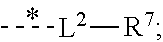
L² is selected from the group consisting of 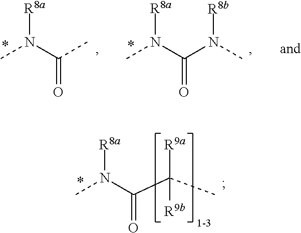
R⁷ is selected from the group consisting of 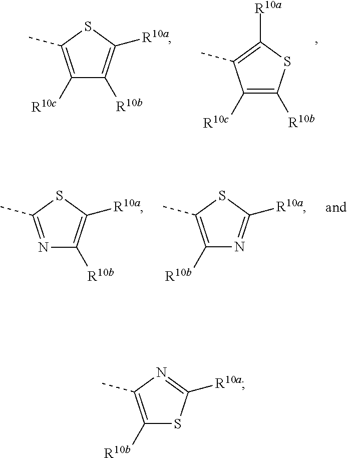
R^{8a} and R^{8b} are each independently selected from the group consisting of H, optionally substituted C₁-C₆ alkyl, and optionally substituted C₃-C₈ cycloalkyl, or R^{8a} and R^{8b} can combine with the atoms to which they are bound to form an optionally substituted C₂-C₈ heterocycloalkyl;
R^{9a} and R^{9b} are each independently selected from the group consisting of H and optionally substituted C₁-C₆ alkyl;
R^{10a}, R^{10b}, and R^{10c} are each independently selected from the group consisting of optionally substituted C₁-C₆ alkyl, optionally substituted C₃-C₈ cycloalkyl, optionally substituted C₆-C₁₀ aryl, optionally substituted C₆-C₈ heteroaryl, halogen, C(═O)R^{A}, C(═O)OR^{A}, C(═O)N(R^{A})(R^{B}), S(═O)R^{A}, S(═O)₂R^{A}, S(═O)N(R^{A})(R^{B}), and S(═O)₂N(R^{A})(R^{B}), or two vicinal substituents selected from the group consisting of R^{10a}, R^{10b}, and R^{10c} can combine with the atoms to which they are bound to form an optionally substituted C₃-C₈ cycloalkyl, optionally substituted C₂-C₈ heterocycloalkyl, optionally substituted C₆-C₁₀ aryl, or optionally substituted C₂-C₈ heteroaryl;
each occurrence of R^{A} and R^{B} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
wherein one of the following applies:
(a) R^{6a} and R^{6b} are each optionally substituted C₁-C₆ alkyl, wherein no more than one of R^{6a} and R^{6b} is optionally substituted C₁ alkyl;
(b) one of R^{6a} and R^{6b} is methyl, and one of R^{6a} and R^{6b} is selected from the group consisting of optionally substituted C₂-C₆ alkenyl, optionally substituted C₂-C₆ alkynyl, optionally substituted C₃-C₈ cycloalkyl, optionally substituted phenyl, and C₁-C₆ alkyl, wherein the optionally substituted alkyl is substituted with at least one optionally substituted phenyl;
(c) one of R^{6a} and R^{6b} is methyl, one of R^{6a} and R^{6b} is H, and one of R^{3a} and R^{3c} is selected from the group consisting of optionally substituted C₁-C₆ alkyl, optionally substituted C₂-C₆ alkenyl, optionally substituted C₂-C₆ alkynyl, optionally substituted C₃-C₈ cycloalkyl, optionally substituted phenyl, and C₁-C₆ alkyl;
(d) one of R^{6a} and R^{6b} is H, and one of R^{6a} and R^{6b} is selected from the group consisting of C₁-C₆ alkyl, optionally substituted C₁-C₆ haloalkoxy, optionally substituted C₂-C₆ alkenyl, optionally substituted C₂-C₆ alkynyl, and C(═O)H, wherein the C₁-C₆ alkyl is substituted with at least one substituent selected from the group consisting of C₂-C₈ alkynyl, C₁-C₆ alkoxy, C═O, and CN; and
(e) one of R^{6a} and R^{6b} is

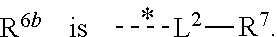

7. The compound of claim 6, wherein R¹ is selected from the group consisting of 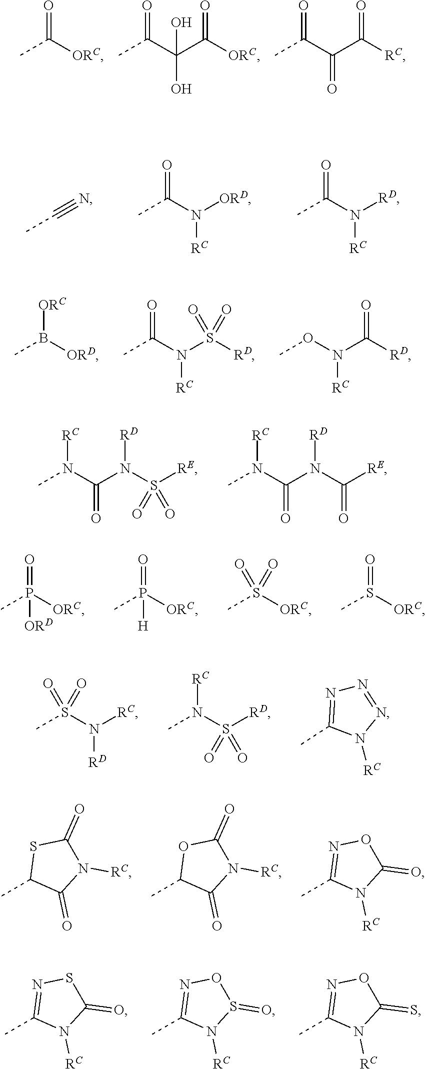 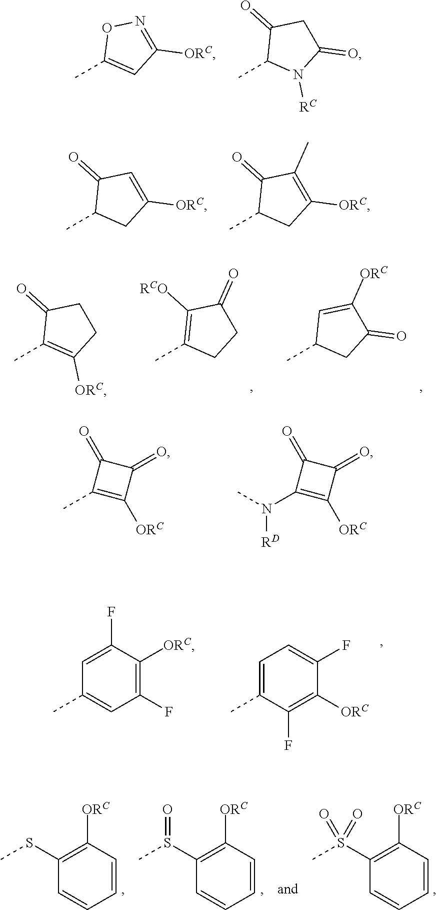
wherein R^{C}, R^{D}, and R^{E}, if present, are each independently selected from the group consisting of H, optionally substituted C₁-C₆ alkyl, and C(═O)(optionally substituted C₁-C₆ alkyl).

8. The compound of claim 6, wherein at least one of the following applies:
(a) R¹ is selected from the group consisting of 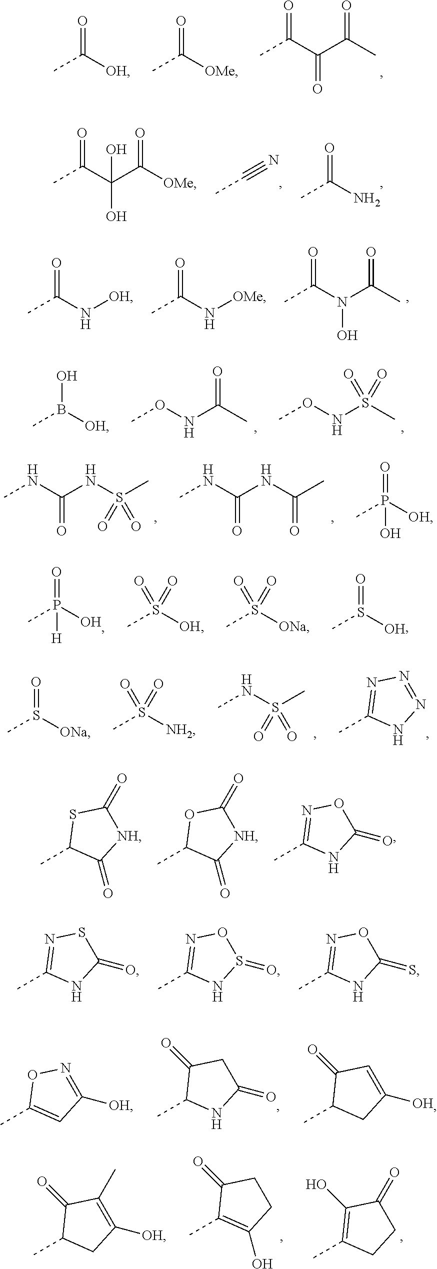 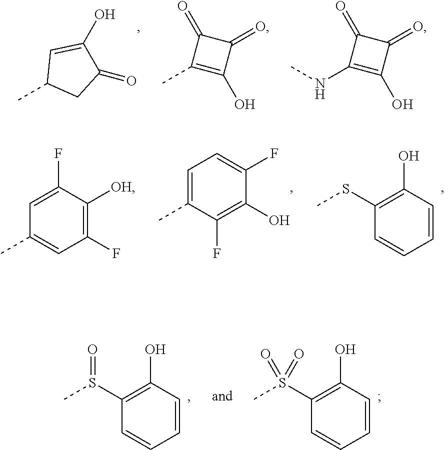
(b) L is a bond;
(c) R^{3a}, R^{3b}, and R^{3c} are each independently selected from the group consisting of H, CH₃ 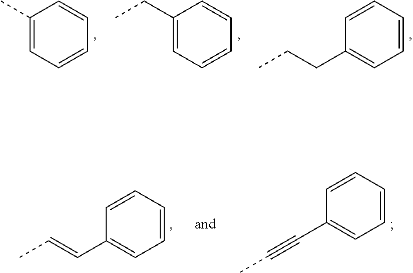
and
(d) either: R^{6a} is ethyl and R^{6b} is methyl, R^{6a} is ethyl and R^{6b} is ethyl, or R^{6a} is methyl and R^{6b} is ethyl.

9. The compound of claim 6, wherein 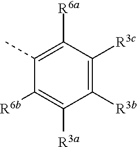
is selected from the group consisting of

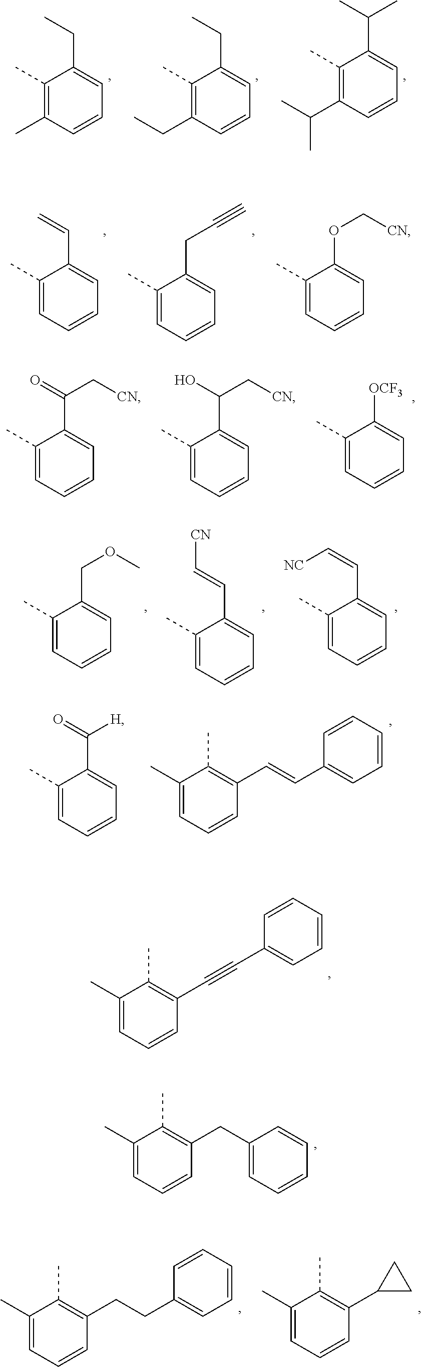

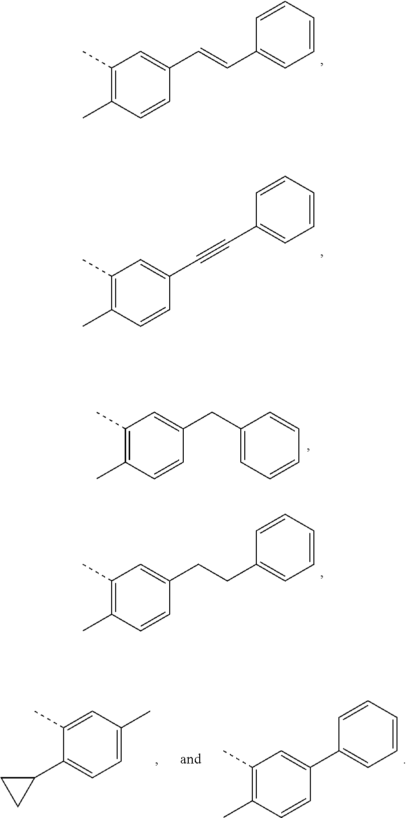

10. The compound of claim 6, wherein R^{6a} is

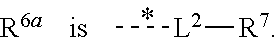

11. The compound of claim 10, wherein R^{6b} is selected from the group consisting of H, CH₃, CF₃, and CH═CH₂.

12. The compound of claim 10, wherein 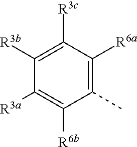
is selected from the group consisting of

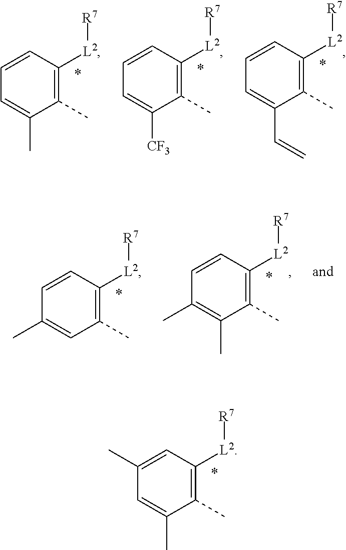

13. The compound of claim 10, wherein R^{8a} and R^{8b} are each independently selected from the group consisting of H, Me, Et, CH₂CN, CH₂C≡CH, iPr, nPr, nBu, 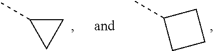
or R^{8a} and R^{gb} combine with the atoms to which they are bound to form

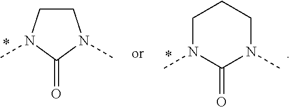

14. The compound of claim 10, wherein L² is selected from the group consisting of

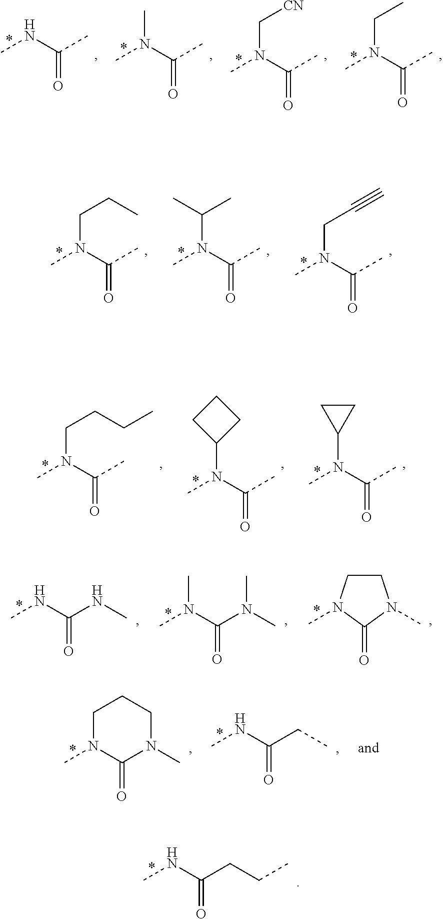

15. The compound of claim 10, wherein R^{10a}, R^{10b}, and R^{10c} are each independently selected from the group consisting of H, Me, CF₂H, CF₃, Et, tBu, F, C₁, OMe, CN, C(═O)OH, C(═O)OMe, C(═O)NH₂, C(═O)Me, S(═O)₂Me,

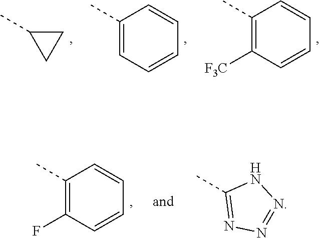

16. The compound of claim 10, wherein R⁷ is selected from the group consisting of

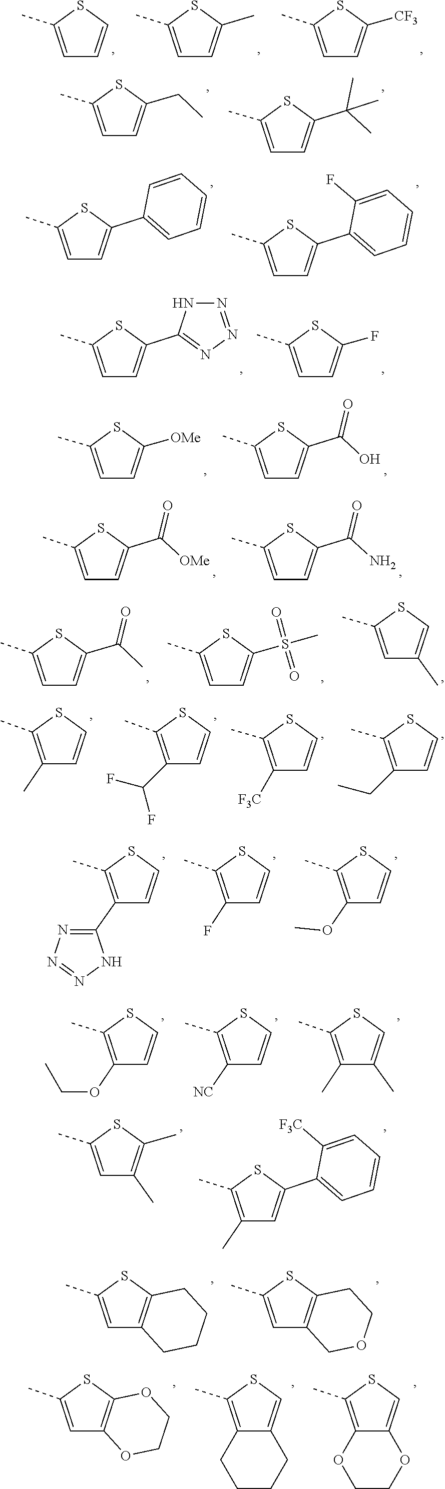

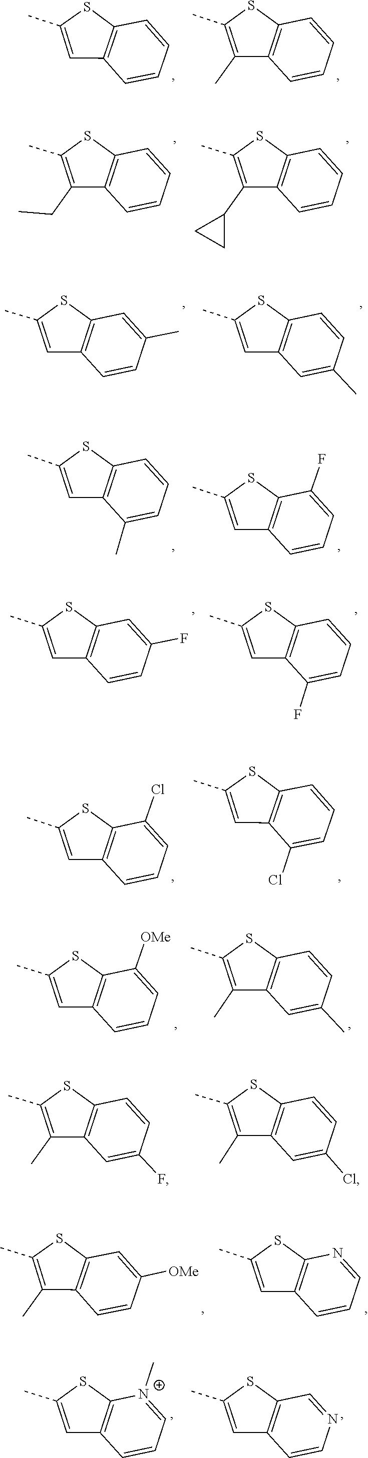

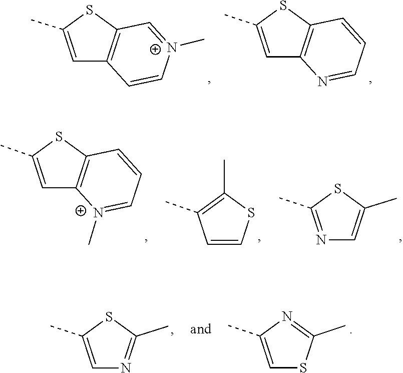

17. The compound of claim 6, which is selected from the group consisting of:
5-(2-ethyl-6-methylphenyl)thiophene-2-carboxylic acid;
5-(2,6-diethylphenyl)thiophene-2-carboxylic acid;
5-(2,6-diisopropylphenyl)thiophene-2-carboxylic acid;
5-(2-vinylphenyl)thiophene-2-carboxylic acid;
5-(2-(2-cyano-1-hydroxyethyl)phenyl)thiophene-2-carboxylic acid;
5-(2-(prop-2-yn-1-yl)phenyl)thiophene-2-carboxylic acid;
5-(2-(trifluoromethoxy)phenyl)thiophene-2-carboxylic acid;
5-(2-(methoxymethyl)phenyl)thiophene-2-carboxylic acid;
5-(2-(2-cyanoacetyl)phenyl)thiophene-2-carboxylic acid;
5-(2-(cyanomethoxy)phenyl)thiophene-2-carboxylic acid;
1-(5-(2,6-diethylphenyl)thiophen-2-yl)butane-1,2,3-trione;
(E)-5-(2-(2-cyanovinyl)phenyl)thiophene-2-carboxylic acid;
(Z)-5-(2-(2-cyanovinyl)phenyl)thiophene-2-carboxylic acid;
5-(2-formylphenyl)thiophene-2-carboxylic acid;
5-(2,6-diethylphenyl)-N-hydroxythiophene-2-carboxamide;
5-(2,6-diethylphenyl)-N-methoxythiophene-2-carboxamide;
5-(2-methyl-5-(phenylethynyl)phenyl)thiophene-2-carboxylic acid;
(E)-5-(2-methyl-5-styrylphenyl)thiophene-2-carboxylic acid;
5-(2-methyl-5-phenethylphenyl)thiophene-2-carboxylic acid;
5-(5-benzyl-2-methylphenyl)thiophene-2-carboxylic acid;
5-(4-methyl-[1,1′-biphenyl]-3-yl)thiophene-2-carboxylic acid;
5-(5-cyclopropyl-2-methylphenyl)thiophene-2-carboxylic acid;
5-(2-benzyl-6-methylphenyl)thiophene-2-carboxylic acid;
5-(2-methyl-6-(phenylethynyl)phenyl)thiophene-2-carboxylic acid;
(E)-5-(2-methyl-6-styrylphenyl)thiophene-2-carboxylic acid;
5-(2-methyl-6-phenethylphenyl)thiophene-2-carboxylic acid;
5-(2-cyclopropyl-5-methylphenyl)thiophene-2-carboxylic acid;
5-(2-(N,5-dimethylthiophene-2-carboxamido)-6-methylphenyl)thiophene-2-carboxylic acid;
5-(2-(benzo[b]thiophene-2-carboxamido)-6-methylphenyl)thiophene-2-carboxylic acid;
5-(2-methyl-6-(6-methylbenzo[b]thiophene-2-carboxamido)phenyl)thiophene-2-carboxylic acid;
5-(2-methyl-6-(N-methylbenzo[b]thiophene-2-carboxamido)phenyl)thiophene-2-carboxylic acid;
5-(2-(N,6-dimethylbenzo[b]thiophene-2-carboxamido)-6-methylphenyl)thiophene-2-carboxylic acid;
5-(2-methyl-6-(2-(thiophen-2-yl)acetamido)phenyl)thiophene-2-carboxylic acid;
5-(2-methyl-6-(5-methylfuran-2-carboxamido)phenyl)thiophene-2-carboxylic acid;
5-(2-methyl-6-(3-methylthiophene-2-carboxamido)phenyl)thiophene-2-carboxylic acid;
5-(2-(N,3-dimethylthiophene-2-carboxamido)-6-methylphenyl)thiophene-2-carboxylic acid;
5-(2-methyl-6-(5-methylthiazole-2-carboxamido)phenyl)thiophene-2-carboxylic acid;
5-(2-(N,5-dimethylthiazole-2-carboxamido)-6-methylphenyl)thiophene-2-carboxylic acid;
5-(2-methyl-6-(4-methylthiophene-2-carboxamido)phenyl)thiophene-2-carboxylic acid;
5-(2-(N-butyl-5-methylthiophene-2-carboxamido)-6-methylphenyl)thiophene-2-carboxylic acid;
5-(2-methyl-6-(2-methylthiazole-4-carboxamido)phenyl)thiophene-2-carboxylic acid;
5-(2-methyl-6-(2-methylthiazole-5-carboxamido)phenyl)thiophene-2-carboxylic acid;
5-(2-methyl-6-(2-methylthiophene-3-carboxamido)phenyl)thiophene-2-carboxylic acid;
5-(2-(N,2-dimethylthiazole-4-carboxamido)-6-methylphenyl)thiophene-2-carboxylic acid;
5-(2-methyl-6-(thiophene-2-carboxamido)phenyl)thiophene-2-carboxylic acid5-(2-methyl-6-(thiophene-2-carboxamido)phenyl)thiophene-2-carboxylic acid;
5-(2-methyl-6-(N-methylthiophene-2-carboxamido)phenyl)thiophene-2-carboxylic acid;
5-(2-(N-ethyl-5-methylthiophene-2-carboxamido)-6-methylphenyl)thiophene-2-carboxylic acid;
5-(2-methyl-6-(3-(5-methylthiophen-2-yl)ureido)phenyl)thiophene-2-carboxylic acid5-(2-methyl-6-(3-(5-methylthiophen-2-yl)ureido)phenyl)thiophene-2-carboxylic acid;
5-(2-methyl-6-(5-methyl-N-propylthiophene-2-carboxamido)phenyl)thiophene-2-carboxylic acid;
5-(2-(N-cyclobutyl-5-methylthiophene-2-carboxamido)-6-methylphenyl)thiophene-2-carboxylic acid;
5-(2-(N,5-dimethylthiophene-2-carboxamido)-6-vinylphenyl)thiophene-2-carboxylic acid;
5-(2-(N,5-dimethylthiophene-2-carboxamido)-6-ethylphenyl)thiophene-2-carboxylic acid;
5-(2-methyl-6-(3-(5-methylthiophen-2-yl)-2-oxoimidazolidin-1-yl)phenyl)thiophene-2-carboxylic acid;
5-(2-(5-ethylthiophene-2-carboxamido)-6-methylphenyl)thiophene-2-carboxylic acid5-(2-(5-ethylthiophene-2-carboxamido)-6-methylphenyl)thiophene-2-carboxylic acid;
5-(2-(5-ethyl-N-methylthiophene-2-carboxamido)-6-methylphenyl)thiophene-2-carboxylic acid;
5-(2-(5-methoxythiophene-2-carboxamido)-6-methylphenyl)thiophene-2-carboxylic acid5-(2-(5-methoxythiophene-2-carboxamido)-6-methylphenyl)thiophene-2-carboxylic acid;
5-(2-(5-methoxy-N-methylthiophene-2-carboxamido)-6-methylphenyl)thiophene-2-carboxylic acid;
5-((2-(5-carboxythiophen-2-yl)-3-methylphenyl)carbamoyl)thiophene-2-carboxylic acid;
5-(2-(5-carboxy-N-methylthiophene-2-carboxamido)-6-methylphenyl)thiophene-2-carboxylic acid;
5-(2-(5-(tert-butyl)thiophene-2-carboxamido)-6-methylphenyl)thiophene-2-carboxylic acid;
5-(2-(5-fluorothiophene-2-carboxamido)-6-methylphenyl)thiophene-2-carboxylic acid5-(2-(5-fluorothiophene-2-carboxamido)-6-methylphenyl)thiophene-2-carboxylic acid;
5-(2-methyl-6-(4-methylbenzo[b]thiophene-2-carboxamido)phenyl)thiophene-2-carboxylic acid;
5-(2-(4,5-dimethylthiophene-2-carboxamido)-6-methylphenyl)thiophene-2-carboxylic acid;
5-(2-(6,7-dihydro-4H-thieno[3,2-c]pyran-2-carboxamido)-6-methylphenyl)thiophene-2-carboxylic acid5-(2-(6,7-dihydro-4H-thieno[3,2-c]pyran-2-carboxamido)-6-methylphenyl)thiophene-2-carboxylic acid;
5-(2-methyl-6-(3-methylbenzo[b]thiophene-2-carboxamido)phenyl)thiophene-2-carboxylic acid;
5-(2-methyl-6-(5-(trifluoromethyl)thiophene-2-carboxamido)phenyl)thiophene-2-carboxylic acid;
5-(2-methyl-6-(4,5,6,7-tetrahydrobenzo[b]thiophene-2-carboxamido)phenyl)thiophene-2-carboxylic acid;
5-(2-methyl-6-(N,4,5-trimethylthiophene-2-carboxamido)phenyl)thiophene-2-carboxylic acid5-(2-methyl-6-(N,4,5-trimethylthiophene-2-carboxamido)phenyl)thiophene-2-carboxylic acid;
5-(2-(5-fluoro-N-methylthiophene-2-carboxamido)-6-methylphenyl)thiophene-2-carboxylic acid;
5-(2-(N,4-dimethylbenzo[b]thiophene-2-carboxamido)-6-methylphenyl)thiophene-2-carboxylic acid;
5-(2-methyl-6-(N,3,4-trimethylthiophene-2-carboxamido)phenyl)thiophene-2-carboxylic acid;
5-(2-methyl-6-(N-methyl-4,5,6,7-tetrahydrobenzo[b]thiophene-2-carboxamido)phenyl)thiophene-2-carboxylic acid;
5-(2-methyl-6-(3-methyl-N-propylthiophene-2-carboxamido)phenyl)thiophene-2-carboxylic acid;
5-(2-(N,3-dimethylbenzo[b]thiophene-2-carboxamido)-6-methylphenyl)thiophene-2-carboxylic acid;
5-(2-methyl-6-(N-methyl-2,3-dihydrothieno[2,3-b][1,4]dioxine-6-carboxamido)phenyl)thiophene-2-carboxylic acid;
5-(2-(N,5-dimethylbenzo[b]thiophene-2-carboxamido)-6-methylphenyl)thiophene-2-carboxylic acid;
5-(2-(3,4-dimethylthiophene-2-carboxamido)-6-methylphenyl)thiophene-2-carboxylic acid;
5-(2-methyl-6-(5-methylbenzo[b]thiophene-2-carboxamido)phenyl)thiophene-2-carboxylic acid;
5-(2-(5-acetylthiophene-2-carboxamido)-6-methylphenyl)thiophene-2-carboxylic acid;
5-(2-methyl-6-(4,5,6,7-tetrahydrobenzo[c]thiophene-1-carboxamido)phenyl)thiophene-2-carboxylic acid;
5-(2-methyl-6-(N-methyl-4,5,6,7-tetrahydrobenzo[c]thiophene-1-carboxamido)phenyl)thiophene-2-carboxylic acid;
5-(2-(N-(cyanomethyl)-5-methylthiophene-2-carboxamido)-6-methylphenyl)thiophene-2-carboxylic acid;
5-(2-methyl-6-(5-methyl-N-(prop-2-yn-1-yl)thiophene-2-carboxamido)phenyl)thiophene-2-carboxylic acid;
5-(2-(N-isopropyl-5-methylthiophene-2-carboxamido)-6-methylphenyl)thiophene-2-carboxylic acid
5-(2-(N-cyclopropyl-5-methylthiophene-2-carboxamido)-6-methylphenyl)thiophene-2-carboxylic acid;
5-(2-(5-methylthiophene-2-carboxamido)-6-(trifluoromethyl)phenyl)thiophene-2-carboxylic acid;
5-(2-(N,5-dimethylthiophene-2-carboxamido)-6-(trifluoromethyl)phenyl)thiophene-2-carboxylic acid;
N-(2-(5-(1H-tetrazol-5-yl)thiophen-2-yl)-3-methylphenyl)-N,5-dimethylthiophene-2-carboxamide;
N-(2-(5-(1H-tetrazol-5-yl)thiophen-2-yl)-3-methylphenyl)-N,3-dimethylthiophene-2-carboxamide;
5-((3-methyl-2-(thiophen-2-yl)phenyl)carbamoyl)thiophene-2-carboxylic acid;
5-(methyl(3-methyl-2-(thiophen-2-yl)phenyl)carbamoyl)thiophene-2-carboxylic acid;
5-((2-(5-carbamoylthiophen-2-yl)-3-methylphenyl)(methyl)carbamoyl)thiophene-2-carboxylic acid;
methyl 5-((2-(5-(1H-tetrazol-5-yl)thiophen-2-yl)-3-methylphenyl)(methyl)carbamoyl)thiophene-2-carboxylate;
5-((2-(5-(1H-tetrazol-5-yl)thiophen-2-yl)-3-methylphenyl)(methyl)carbamoyl)thiophene-2-carboxylic acid;
methyl 5-((2-(5-(methoxycarbonyl)thiophen-2-yl)-3-methylphenyl)carbamoyl)thiophene-2-carboxylate;
N-(2-(5-(1H-tetrazol-5-yl)thiophen-2-yl)-3-methylphenyl)-N,3-dimethylbenzo[b]thiophene-2-carboxamide;
5-(2-(5-carbamoyl-N-methylthiophene-2-carboxamido)-6-methylphenyl)thiophene-2-carboxylic acid;
5-(2-methyl-6-(N-methyl-5-(1H-tetrazol-5-yl)thiophene-2-carboxamido)phenyl)thiophene-2-carboxylic acid;
5-(2-(5-acetyl-N-methylthiophene-2-carboxamido)-6-methylphenyl)thiophene-2-carboxylic acid;
5-(2-methyl-6-(N-methyl-5-(trifluoromethyl)thiophene-2-carboxamido)phenyl)thiophene-2-carboxylic acid;
5-(2-(3-ethylbenzo[b]thiophene-2-carboxamido)-6-methylphenyl)thiophene-2-carboxylic acid;
5-(2-methyl-6-(3-(trifluoromethyl)thiophene-2-carboxamido)phenyl)thiophene-2-carboxylic acid;
5-(2-(5-carboxy-N-methylthiophene-2-carboxamido)-4,6-dimethylphenyl)thiophene-2-carboxylic acid;
5-(2-(3-fluorothiophene-2-carboxamido)-6-methylphenyl)thiophene-2-carboxylic acid;
5-(2-(3-cyclopropylbenzo[b]thiophene-2-carboxamido)-6-methylphenyl)thiophene-2-carboxylic acid;
5-(2-(3-methoxy-N-methylthiophene-2-carboxamido)-6-methylphenyl)thiophene-2-carboxylic acid;
5-(2-methyl-6-(N-methyl-2,3-dihydrothieno[3,4-b][1,4]dioxine-5-carboxamido)phenyl)thiophene-2-carboxylic acid;
5-(2-(3-ethyl-N-methylthiophene-2-carboxamido)-6-methylphenyl)thiophene-2-carboxylic acid;
5-(2-(3-ethoxy-N-methylthiophene-2-carboxamido)-6-methylphenyl)thiophene-2-carboxylic acid;
5-(2-(3-ethoxythiophene-2-carboxamido)-6-methylphenyl)thiophene-2-carboxylic acid;
methyl 5-(2-methyl-6-(N-methyl-3-(1H-tetrazol-5-yl)thiophene-2-carboxamido)phenyl)thiophene-2-carboxylate;
5-(2-methyl-6-(N-methyl-3-(1H-tetrazol-5-yl)thiophene-2-carboxamido)phenyl)thiophene-2-carboxylic acid;
5-(2-(5-fluoro-3-methylbenzo[b]thiophene-2-carboxamido)-6-methylphenyl)thiophene-2-carboxylic acid;
5-(2-(5-fluoro-N,3-dimethylbenzo[b]thiophene-2-carboxamido)-6-methylphenyl)thiophene-2-carboxylic acid;
5-(2-(3-ethyl-N-methylbenzo[b]thiophene-2-carboxamido)-6-methylphenyl)thiophene-2-carboxylic acid;
5-(2-(5-chloro-N,3-dimethylbenzo[b]thiophene-2-carboxamido)-6-methylphenyl)thiophene-2-carboxylic acid;
5-(2-(3-cyclopropyl-N-methylbenzo[b]thiophene-2-carboxamido)-6-methylphenyl)thiophene-2-carboxylic acid;
5-(2-(3-ethylthiophene-2-carboxamido)-6-methylphenyl)thiophene-2-carboxylic acid;
5-(2-(3-(difluoromethyl)thiophene-2-carboxamido)-6-methylphenyl)thiophene-2-carboxylic acid;
5-(2-(3-(difluoromethyl)-N-methylthiophene-2-carboxamido)-6-methylphenyl)thiophene-2-carboxylic acid;
5-(2-methyl-6-(thieno[2,3-b]pyridine-2-carboxamido)phenyl)thiophene-2-carboxylic acid;
5-(2-methyl-6-(N-methylthieno[2,3-b]pyridine-2-carboxamido)phenyl)thiophene-2-carboxylic acid;
2-((2-(5-carboxythiophen-2-yl)-3-methylphenyl)(methyl)carbamoyl)-7-methylthieno[2,3-b]pyridin-7-ium;
5-(2-methyl-6-(thieno[3,2-b]pyridine-2-carboxamido)phenyl)thiophene-2-carboxylic acid;
5-(2-methyl-6-(N-methylthieno[3,2-b]pyridine-2-carboxamido)phenyl)thiophene-2-carboxylic acid;
2-((2-(5-carboxythiophen-2-yl)-3-methylphenyl)(methyl)carbamoyl)-4-methylthieno[3,2-b]pyridin-4-ium;
5-(2-(7-fluorobenzo[b]thiophene-2-carboxamido)-6-methylphenyl)thiophene-2-carboxylic acid;
5-(2-(7-fluoro-N-methylbenzo[b]thiophene-2-carboxamido)-6-methylphenyl)thiophene-2-carboxylic acid;
5-(2-(3,5-dimethylbenzo[b]thiophene-2-carboxamido)-6-methylphenyl)thiophene-2-carboxylic acid;
5-(2-methyl-6-(N,3,5-trimethylbenzo[b]thiophene-2-carboxamido)phenyl)thiophene-2-carboxylic acid;
5-(2-methyl-6-(5-phenylthiophene-2-carboxamido)phenyl)thiophene-2-carboxylic acid;
5-(2-methyl-6-(N-methyl-5-phenylthiophene-2-carboxamido)phenyl)thiophene-2-carboxylic acid;
2-((2-(5-carboxythiophen-2-yl)-3-methylphenyl)(methyl)carbamoyl)-6-methylthieno[2,3-c]pyridin-6-ium;
5-(2-(N,3-dimethylthiophene-2-carboxamido)-5-methylphenyl)thiophene-2-carboxylic acid;
5-(2-(N,3-dimethylthiophene-2-carboxamido)-5-ethylphenyl)thiophene-2-carboxylic acid;
5-(2-methyl-6-(N-methyl-5-(methylsulfonyl)thiophene-2-carboxamido)phenyl)thiophene-2-carboxylic acid;
5-(2-(5-(2-fluorophenyl)-N-methylthiophene-2-carboxamido)-6-methylphenyl)thiophene-2-carboxylic acid;
5-(2-(4-fluoro-N-methylbenzo[b]thiophene-2-carboxamido)-6-methylphenyl)thiophene-2-carboxylic acid;
5-(2-(7-chloro-N-methylbenzo[b]thiophene-2-carboxamido)-6-methylphenyl)thiophene-2-carboxylic acid;
5-(2-(4-chloro-N-methylbenzo[b]thiophene-2-carboxamido)-6-methylphenyl)thiophene-2-carboxylic acid;
5-(2-(6-fluoro-N-methylbenzo[b]thiophene-2-carboxamido)-6-methylphenyl)thiophene-2-carboxylic acid;
5-(2-(5-methoxy-N,3-dimethylbenzo[b]thiophene-2-carboxamido)-6-methylphenyl)thiophene-2-carboxylic acid;
5-(2-methyl-6-(N-methylthieno[2,3-c]pyridine-2-carboxamido)phenyl)thiophene-2-carboxylic acid;
5-(6-(N,3-dimethylbenzo[b]thiophene-2-carboxamido)-2,3-dimethylphenyl)thiophene-2-carboxylic acid;
5-(2-(7-methoxy-N-methylbenzo[b]thiophene-2-carboxamido)-6-methylphenyl)thiophene-2-carboxylic acid; and
5-(2-(N,3-dimethyl-5-(2-(trifluoromethyl)phenyl)thiophene-2-carboxamido)-6-methylphenyl)thiophene-2-carboxylic acid.

18. A pharmaceutical composition comprising the compound of claim 6 a pharmaceutically acceptable carrier.

19. A method of treating, preventing, and/or ameliorating toxicity caused by a ribosome inactivating protein (RIP) in a subject, the method comprising administering to the subject a therapeutically effective amount of a compound selected from the group consisting of:
(a) a compound of Formula (III) or a pharmaceutically acceptable salt thereof: 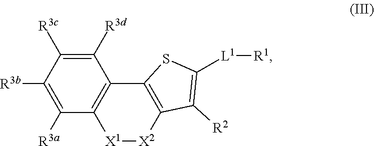
wherein:
R¹ is a carboxylic acid (—COOH), —C(═O)C(OH)₂C(═O)OR^{A}, a carboxylic acid bioisostere, or a carboxylic acid precursor;
R² is H;
R^{3a}, R^{3b}, R^{3c}, and R^{3d} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl, halogen, CN, NO₂, OR^{A}, N(R^{A})(R^{B}), C(═O)R^{A}, C(═O)OR^{A}, C(═O)N(R^{A})(R^{B}), N(R^{A})C(═O)R^{B}, S(═O)₂R^{A}, S(═O)₂N(R^{A})(R^{B}), S(═O)₂OR^{A}, and N(R^{A})S(═O)₂R^{B};
L¹ is selected from the group consisting of a bond and optionally substituted C₁-C₂ alkylenyl;
X¹ is selected from the group consisting of —C(R^{4a})(R^{4b})—, 0, and S;
X² is selected from the group consisting of —C(R^{5a})(R^{5b})— and a bond;
R^{4a} and R^{4b} are each independently selected from the group consisting of H and optionally substituted C₁-C₆ alkyl,
wherein no more than one of R^{4a} and R^{4b} is H;
R^{5a} and R^{5b} are each independently selected from the group consisting of H and optionally substituted C₁-C₆ alkyl,
or R^{5a} and R^{5b} can combine with the carbon atom to which they are bound to form a carbonyl moiety (C═O);
R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₈ heteroaryl;
(b) the compound of claim 6; and
(c) a compound selected from the group consisting of:
5-(4-fluoro-2,6-dimethylphenyl)thiophene-2-carboxylic acid;
5-(2-bromophenyl)thiophene-2-carboxylic acid;
5-(2-cyclohexylphenyl)thiophene-2-carboxylic acid;
5-(2-hydroxyphenyl)thiophene-2-carboxylic acid;
5-(2,3-dimethylphenyl)thiophene-2-carboxylic acid;
5-(2-methoxy-6-methylphenyl)-3-methylthiophene-2-carboxylic acid;
5-(4-amino-2,6-dimethylphenyl)thiophene-2-carboxylic acid;
5-(2-ethyl-6-(5-methylthiophene-2-carbonyl)phenyl)thiophene-2-carboxylic acid;
5-(2-ethyl-6-((5-methylthiophen-2-yl)methyl)phenyl)thiophene-2-carboxylic acid;
5,5-dimethyl-4-oxo-4,5-dihydronaphtho[1,2-b]thiophene-2-carboxylic acid;
5-(2-ethylphenyl)-4-methylthiophene-2-carboxylic acid;
5-(2-ethylphenyl)-1-methyl-1H-pyrazole-3-carboxylic acid;
5-(2-ethylphenyl)-1H-pyrazole-3-carboxylic acid;
methyl 3-(5,5-dimethyl-4,5-dihydronaphtho[1,2-b]thiophen-2-yl)-2,2-dihydroxy-3-oxopropanoate;
methyl 5-(5-bromo-2-methylphenyl)thiophene-2-carboxylate;
1-(4-methyl-3-nitropyridin-2-yl)-4-((4-methylthiazol-2-yl)methyl)piperidin-4-ol;
5-(2-bromo-6-methylphenyl)thiophene-2-carboxylic acid; and
methyl 5-(2-bromo-6-methylphenyl)thiophene-2-carboxylate.

20. A method for measuring binding affinity of a small molecule for an active A chain (RTA) of a ribosome inactivating protein (RIP), the method comprising:
(a) contacting a small molecule with a mixture comprising a fluorescently labeled P11 polypeptide (SEQ ID NO:1) and RTA to provide a displacing mixture;
(b) measuring fluorescence of the displacing mixture to provide a displaced fluorescence mixture;
(c) measuring fluorescence of a control sample comprising the fluorescently labeled P11 polypeptide (SEQ ID NO:1) and RTA, and lacking the small molecule, to provide a control measurement; and
(d) comparing the displaced fluorescence measurement and the control fluorescence measurement.
